(19) United States
(12) Patent Application Publication
(10) Pub. No.: US 2007/0212700 A1
(43) Pub. Date: Sep. 13, 2007

(54) Methods of using and analyzing biological sequence data

(75) Inventors: Rama Ranganathan, Dallas, TX (US); William Russ, Dallas, TX (US); Christopher Larson, Dallas, TX (US); Rohit Sharma, Brookline, MA (US)

Correspondence Address: FULBRIGHT & JAWORSKI L.L.P., 600 CONGRESS AVE., SUITE 2400, AUSTIN, TX, 78701, US

(73) Assignee: THE BOARD OF REGENTS OF THE UNIVERSITY OF TEXAS SYSTEM

(21) Appl. No.: 11/518,590

(22) Filed: Sep. 7, 2006

Related U.S. Application Data
(60) Provisional application No. 60/714,675, filed on Sep. 7, 2005.

(51) Int. Cl. C12Q 1/68; G06F 19/00

(52) U.S. Cl. 435/6; 435/7.1; 702/19; 702/20

(57) ABSTRACT

Methods of using biological sequence data. Evolved biological sequences may be used to identify the defining biological characteristics of the sequences—the three-dimensional structure and biochemical function. Some of the present methods extract such information, use such information to predict functional mechanism, and/or use such information in the design of artificial biological sequences. Other methods are included, as are related computer readable media and computer systems.

### CROSS-REFERENCE(S) TO RELATED APPLICATION(S)

This application claims priority to U.S. Provisional Patent Application Ser. No. 60/714,675, filed Sep. 7, 2005, the entire contents of which are expressly incorporated by reference.

### REFERENCE TO COMPUTER PROGRAM LISTING APPENDIX

This application includes a computer program listing appendix, submitted on compact disc (CD). The content of the CD is incorporated by reference in its entirety and accordingly forms a part of this specification. The CD contains the following files:

| | |
|---|---|
| File name: SCAMC.txt | File Size: 16 KB |
| File name: SCAMCMASK.txt | File Size: 23 KB |
| Creation date for CD: | Sep. 7, 2006 |

The appendix to this specification contains computer-program source code that is the property of the assignee. Copies of the source code may be made as part of making facsimile reproductions of this specification, but all other rights in the source code are reserved. Those with skill in the art having the benefit of this disclosure will understand that the appended source code may be modified as necessary for use with operating systems other than the standard, UNIX-based operating system for which it is currently written. For example, the appended source code may be modified for use with any Microsoft Windows operating system.

### BACKGROUND

1. Field

The present invention relates generally to the use of biological sequence data. For example, evolved biological sequences may be used to identify the defining biological characteristics of the sequences—the three-dimensional structure and biochemical function. The present invention relates to methods of extracting such information, to using such information to predict functional mechanism, and to using such information in the design of artificial biological sequences. The present invention also relates to comparing the functionality and folding of such designed biological sequences to those of known sequences. The present invention relates to computer readable media comprising machine readable instructions for carrying out at least the steps of any of the present methods. The present invention also relates to computing systems (e.g., one or more computers, circuits, or the like) that are programmed or operable to carry out at least the steps of any of the present methods. The present invention also relates to the compositions of matter (e.g., biological sequences) that are designed using one or more of the present methods.

2. Description of Related Art

Classical studies show that for many proteins, the information required for specifying the tertiary structure is contained in the amino acid sequence. However, the potential complexity of this information is enormous, a problem that makes defining the rules for protein folding difficult through either computational or experimental methods.

A fundamental tenet of biochemistry is that the amino acid sequence of a protein specifies its atomic structure and biochemical function (Anfinsen, 1973). But exactly what information in the sequence of a protein is necessary and sufficient for producing the fold and its biological activity is unknown, despite considerable progress in understanding the mechanisms of protein folding (Daggett & Fersht, 2003; Dinner et al., 200; Dobson, 2003; Fersht & Daggett, 2002). The main problem is the vast potential complexity of cooperative interactions between amino acids—processes by which the free energy contribution of one residue depends on those of other residues (Hidalgo & MacKinnon, 1995; Horovitz & Fersht, 1992; Marqusee & Sauer, 1994). These amino acid couplings could be pairwise and local in the three-dimensional structure, but could also involve more complex cooperativities in which collections of residues interact through three-way or higher-order couplings (Chen & Stites, 2001; Klinger & Brutlag, 1994; LiCata & Ackers, 1995; Luque et al., 2002). Given that protein structures are typically compact and well-packed (Gerstein & Chothia, 1996; Richards & Lim, 1993), proteins could be dense and complex networks of inter-atomic interactions, requiring specification of a great number of mutual constraints between amino acid positions to define the fold.

In Suel et al., however, the authors suggest that the pattern of inter-residue interactions seems to be much simpler than expected. The interactions that were analyzed resulted from perturbations that were based on the conservation of specific amino acids within the alignment in question.

### SUMMARY

In one respect, some embodiments of the present methods comprise (a) testing the size and diversity of an alignment of a family of M biological sequences, each biological sequence having N positions, each position being occupied by one biological position element of a group of biological position elements; (b) calculating a statistical conservation value for each biological position element in a pair of biological position elements at different positions in the alignment; (c) measuring conserved co-variation between the biological position elements in the pair using the statistical conservation values.

In another respect, some embodiments of the present methods comprise (a) calculating a statistical conservation value for each biological position element in a pair of biological position elements at different positions in an alignment of a family of M biological sequences, each biological sequence having N positions, and each position being occupied by one biological position element of a group of biological position elements; (b) making a perturbation to the alignment that is not based on the conservation of a particular biological position element at a particular position, the perturbation yielding a subalignment having fewer than M biological sequences; and (c) calculating a statistical conservation value for each biological position element in a pair of biological position elements at the different positions in the subalignment.

Other embodiments of the present methods, along with embodiments of the present computer readable media and computer systems, are presented below.

### BRIEF DESCRIPTION OF THE DRAWINGS

The following drawings illustrate by way of example and not limitation. The patent or application file contains at least one drawing executed in color. Copies of this patent or patent application publication with color drawings will be provided by the Office upon request and payment of the necessary fee.

FIG. 1: A portion of the truncated alignment of WW sequences that has been restricted to have no two sequences with more than 90 percent pairwise identity. Position numbers are indicated above the sequences. Highly conserved positions 7, 30 and 33 are shaded. Sequences shown are SEQ ID NO. 141-160 (listed from top to bottom).

FIG. 2: Conservation pattern for the WW domain family. The magnitude of ΔE_{i,x}^{stat} values (described below) for all amino acids x is shown for each position i. Weakly conserved positions show low ΔE^{stat} values, whereas highly conserved positions (such as 7, 30 and 33, see FIG. 1) show high ΔE^{stat} values. Three moderately conserved positions (8, 23 and 29) are indicated by arrows. Sequence alignments for the WW domain family members are shown in FIGS. 43A-I.

FIG. 3: Fluctuation of perturbation vectors for three moderately-conserved positions within the working WW alignment. Shown are perturbation vectors for three positions with similar ΔE_{i,x}^{stat} values: glutamic acid at position 8, histidine at position 23, and threonine at position 29 (see arrows in FIG. 2).

FIG. 4: Evolutionary coupling in the WW domain. The magnitude of C_{i,x,j,y}^{ev} values (described below) for all amino acids x and y are shown for each pair of positions i and j, which are the rows and columns of the statistical coupling matrix (SCM) shown. The positions in the rows and columns are organized from N- to C-terminus going left to right, and from top to bottom. The secondary structure of the WW domain is indicated at the top and left, with arrows representing β-sheets. The C_{i,j}^{ev} values are represented on a color scale (right) from blue (0) to red (2). Circles indicate the C_{i,j}^{ev} between positions 8, 23 and 29.

FIG. 5: Clustering of the data in the SCM shown in FIG. 4. The C_{i,j}^{ev} values in the SCM matrix were clustered in both dimensions and re-displayed on a colorscale from blue (0) to red (2). The dendrogram at right indicates the linkage between matrix elements/locations (which represent position pairs). The sort order is indicated by the list of WW alignment (or sequence) positions next to the dendrogram. The numbering of the columns of the clustered matrix are identical to the numbering of the rows (such that the leftmost row is 13, and the rightmost row is 23). A single cluster, or group, of matrix elements comprising positions 3, 4, 6, 8, 21, 22, 23 and 28 of the WW alignment is separated from the rest by a primary root branch. These positions all have high coupling scores with similar patterns of evolutionary coupling to each other, and therefore comprise a network of evolutionarily-conserved couplings.

FIGS. 6A-C: A spatially distributed network underlying WW specificity. FIG. 6A: two views of the Nedd4.3 WW domain (in blue CPK), with residues comprising the cluster of eight co-evolving residues as red CPK with a transparent van der Waals surface. The cluster forms a physically connected network that links binding site residues at positions 21, 23, and 28 with the opposite side residues at positions 3 and 4 through a few intervening residues at positions 6, 8, and 22. FIG. 6B: the thermodynamic mutant cycle formalism for measuring energetic coupling between a pair of mutations. The method involves measuring equilibrium dissociation constants for peptide binding for four proteins: wild-type (WT), a mutation at one site (M1), a mutation at a second site (M2), and the double mutation M1, M2. The fold effect of M1 is calculated for the wild type background (X1) or for the background of the second mutation (X2). The thermodynamic coupling parameter Ω is defined as the ratio of these two fold effects (X1/X2)— the degree to which the effect of one mutation depends on the second. FIG. 6C: double mutant cycle analysis of co-evolving positions in the N39 (Nedd4.3) WW domain. The residues at positions 3, 8, 23, and 28 are shown in the same orientation as in panel A (right), with distances marked between β carbons of residues indicated by dashed lines. Alanine mutations at three of these sites (3, 8, 23) were made both singly and in combinations with an alanine mutation at 28. Peptide binding to wild-type and mutant WW domains was measured using isothermal titration calorimetery. Single mutations at all sites affect peptide binding (fold effect relative to wild-type in parentheses), and mutant cycle analysis demonstrates energetic coupling (Ω>1) between position 28 and positions 3, 8, and 23. Panels A and C were prepared with PyMOL (Delano, 2002).

FIG. 7: Conservation pattern for the PDZ domain family. Sequence alignments of the natural PDZ domains are shown in FIGS. 45A-E.

FIG. 8: The reduced cmr matrix (“cmr” is defined below) of C_{i,j}^{ev} values. The matrix is organized with rows and columns representing positions in the alignment from N- to C-terminus of the sequence. The secondary structure of the PDZ domain is shown on the top and left, with arrows representing β-sheets. The C_{i,j}^{ev} values are represented on a color scale (right) from blue (0) to red (1.2).

FIGS. 9A-C: Results of one version of the present statistical coupling analysis (SCA) for PDZ domains. FIG. 9A: the clustered cmr matrix, with C_{i,j}^{ev} values shown on the color scale shown in FIG. 8. Iterative clustering (Suel et al.) was used to condense the high coupling signals to a single branch of the dendrogram. Specifically, clustering was performed three times—rows and columns with strong signals were isolated from the initial clustered matrix (large white box) and re-clustered, causing the strong couplings to cluster inside the small white box. These rows and columns were re-clustered again, resulting in the dendrogram shown at the right. Sequence numbering is consistent with that reported in Lockless et al. (1999). FIGS. 9B and 9C: mapping the clusters of high coupling shows two contacting networks that line the base of the peptide binding pocket, continue through the core, and extend to the back surface on helix αA. The bottom cluster is mapped in FIG. 9B, and the upper group in FIG. 9C.

FIG. 10: Two-by-two contingency matrix for testing statistical significance of functional predictions in the PDZ domain using an embodiment of the present SCA. Of the 36 mutants tested by Skelton et al. (2003) that were in the alignment, 12 are part of the coupled network and 23 are not. Their results showed that of 13 mutants that had a significant effect on binding, 10 were on the network that was identified. Of the 23 that had no effect, only 2 were on the network that was identified. Using Fisher's Exact test, these results are significant at p=0.00006.

FIG. 11: Interaction of CDC42 with Par6. The crystal structure of CDC42 (grey space-filling model) bound to the Par6 PDZ domain (green cartoon) is shown (PDB accession 1NF3). The side chains of the strongly coupled network is shown as salmon space-filling. The network connects the Par6 interaction site with the peptide binding site.

FIG. 12: Conservation pattern of the caspase family.

FIGS. 13A-B: Results of one version of SCA for the caspase family of cysteine proteases. FIG. 13A: the cmr matrix is represented as a color scale from red to blue. FIG. 13B: heirarchical clustering reveals two sets of biological sequence positions with strong coupling values. The bottom cluster (red dendrogram) comprises positions 74, 78, 87, 131, 143, 152, 166, 171, 177, 178, 179, 184, 188, 218, 233, and 240. The upper cluster (blue dendrogram) comprises positions 68, 70, 72, 75, 90, 92, 97, 101, 104, 108, 140, 141, 142, 174, 181, 183, 185, 187, 214, 219, 223, 224, 225, 229, 232, 238, 239, 242, 245, 246, 265, 266, and 295. The positions numbers are the positions in the human caspase-7 protein, as numbered in the PDB file 1SHJ.

FIGS. 14A-F: A network of evolutionarily-coupled residues in the caspase family. FIG. 14A: the lower and upper strongly co-evolving clusters (red and blue surfaces, respectively) from FIG. 13B are mapped on the structure of human caspase-7 (PDB accession 1SHJ). The allosteric ligand, 2-(2,4-Dichlorophenoxy)-N-(2-mercapto-ethyl)-acetamid (DICA), is shown in orange space-filling. Protamer A (left) is shown as a cartoon representation, and protamer B (right) is shown in space-filling, indicating that the coupled residues are mostly buried. The active site cysteine is shown in green. FIGS. 14B-F: rotations of the right protamer shown in FIG. 14A, to highlight the limited solvent accessibility of the coupled network. FIGS. 14B-C show the bottom and top of the view shown in FIG. 14A. FIGS. 14D-F are 90° rotations about the vertical axis. The most extensive accessible surfaces are in the active site (FIG. 14B) and at the DICA binding site (FIG. 14D, DICA shown as orange sticks).

FIG. 15: Conservation pattern of the glycogen phosphorylase family. Several sites have very low conservation, indicating that the alignment is at statistical equilibrium.

FIGS. 16A-B: Results of one version of SCA for the glycogen phosphorylase family. The cmr matrix is shown on a colorscale from blue (0) to red (2.5) in both unclustered (FIG. 16A) and clustered (FIG. 16B) arrangements.

FIGS. 17A-F: Mapping of SCA results shown in FIG. 16B onto the structure of human liver glycogen phosphorylase B. FIG. 17A: the strongly co-evolving network (blue dendrogram from FIG. 16B) is shown as a blue surface. The left protamer is shown as a cartoon, and the left protamer as a space-filling model. Ligands are shown with space-filling atoms as well; PLP (an essential co-factor) in red, caffeine in cyan, AMP in pink, glucose in green, and the drug CP-403,700 in orange. Glucose lies in the active site, which is surrounded by the coupled network. The network also makes direct contact with all of the other ligands. FIGS. 17B-C show the bottom and top of the right protamer as shown in FIG. 17A. FIGS. 17D-F show views of the right protamer in FIG. 17A, in 90° rotations about the vertical axis. The structure is drawn from PDB accession 1EXV, except for the AMP ligand, which is overlayed from accession 1FA9.

FIGS. 18A-B: Vertical shuffling of the alignment destroys pairwise coupling. FIG. 18A: the cmr matrix for the working WW alignment. FIG. 18B: the cmr matrix for the vertically-shuffled alignment. Nearly 90,000 swaps were made between randomly-selected pairs of sequences at a randomly-selected position in the alignment. Both matrices have been sorted by rr_cluster_{—}2.m (provided below) to make visualization easier.

FIG. 19: Energy trajectory for the Monte Carlo simulation of WW domains. The system energy, eₙ, is plotted as a function of β (energy line). As the energy converges to a minimum value, the top-hit pairwise identity to natural WW domains increases, to a maximum value of 0.84. Vertical bars indicate points along the trajectory from which alignments were taken, at maximum identities of 52%, 55%, 60%, 70%, 80% (having corresponding eₙ values of 18114, 12602, 8171, 4528, and 2721) and at the final convergence point at 84% identity (having a corresponding eₙ value of 2116).

FIGS. 20A-F: Coupling recovers over the course of the Monte Carlo run. FIGS. 20A-F show cmr matrices on a color scale from blue (0) to red (2) for the maximum pairwise identities of 52%, 55%, 60%, 70%, 80% and 84%, respectively, shown in FIG. 19. Each matrix is labeled with the average maximum percent identity to natural WW domains (% ID) and energy (eₙ) of the alignment.

FIGS. 21A-C: Experimental evaluation of artificial proteins. Thermal denaturation studies for a representative sampling of WW sequences drawn from the natural alignment (FIG. 21A), the alignment with conservation preserved, but no coupling (IC, FIG. 21B), and for the artificial sequences drawn along the Monte Carlo trajectory at 60% maximal identity to natural WW domains (CC60 FIG. 21C). For natural and CC60 sequences, three sequences are shown that were highly stable, moderately stable, and unfolded. For IC sequences, melts of every soluble sequence (N=30) are overlaid in gray. Sequences are scored as natively folded if they show cooperative and reversible thermal denaturation. While natural and CC sequences include some that are folded, no IC sequences are folded. The sequence alignments of the natural WW domains used in the Monte Carlo method to generate the artificial sequences are shown in FIGS. 44A-C.

FIGS. 22A-F: Representative thermal denaturation curves for all sets of artificial sequences. Two folded domains were chosen from each set. FIG. 22A, CC52 (SEQ ID NO:1-20), FIG. 22bB CC55 (SEQ ID NO:21-40), FIG. 22C, CC60 (SEQ ID NO:41-60), FIG. 22D, CC70 (SEQ ID NO:61-80), FIG. 22E, CC80 (SEQ ID NO:81-100) and FIG. 22F, the fully converged set (CCconv) (SEQ ID NO:101-120). For each domain, forward (upper line in each plot) and reverse (lower line in each plot) melts are shown, where the temperature is increased from 4° C. to 90° C., or decreased from 90° C. to 4° C., respectively. In all cases shown, the denaturation curves show proteins that are stable and largely reversible, indicating natively folded proteins.

FIG. 23: Thermodynamic parameters for natural and artificial WW domains. The melting temperature (Tm) and change in enthalpy upon unfolding (ΔH) extracted from the melting curves is shown for all folded domains from each set: natural WW domains, open circles; CC52, purple; CC55, blue; CC60, green; CC70, orange; CC80, red; fully converged, black.

FIG. 24: The peptide binding surface of the WW domain contains two structurally-defined pockets, the X-Pro binding site (residues 19 and 30, in blue CPK), and a specificity site (residues 21, 23, and 26, in yellow). Shown is a ribbon and transparent molecular surface representation of the Nedd4.3 WW domain bound to a group I peptide (in green stick bonds, PDB 115H). The figure was prepared with PyMOL (Delano, 2002).

FIGS. 25A-D: Assays for binding affinity and specificity in WW domains. FIG. 25A: five N-terminal biotinylated oriented peptide libraries were constructed to present either a proline-only control (biotin-Z-GMAxxxxPxxxxAKKK (SEQ ID NO:162)) or the four different characteristic WW domain binding motifs: group I-oriented (biotin-Z-GMAxxxPPxYxxxAKKK-C (SEQ ID NO:163)), group II-oriented (biotin-Z-GMAxxxPPLPxxxAKKK (SEQ ID NO:164)), group III-oriented (biotin-Z-GMAxxxPPRxxxAKKK (SEQ ID NO:165)), and group IV-oriented (biotin-Z-GMAxxxxpSPxxxxAKKK (SEQ ID NO:166)), where Z is 6-aminohexanoic acid, pS is phosphoserine, and x denotes all amino acids except Cys. Binding to these peptides indicte affinity for the consensus motifes listed in FIG. 25A (SEQ ID NO:167-171). FIG. 25B: binding specificity of natural WW domains exhibiting four binding class-specificities to the peptide libraries in FIG. 25A. FIG. 25C: binding specificity of artificial WW domains from the CC55 set. Binding is reported in fold above background binding in the absence of target peptides. Shown are the mean and standard deviation of at least four independent assays. FIG. 25D: the binding specificity of additional artificial and natural WW domains.

FIG. 26 depicts a flowchart showing, in a broad respect, some embodiments of the present methods.

FIG. 27 depicts a flowchart showing, in another broad respect, some embodiments of the present methods.

FIG. 28: Top-hit sequence identity for alignments of artificial SH3 domains generated using the optimization algorithm with masks made at different standard deviation (sigma) cutoff levels. Points with errorbars indicate the mean and standard deviation of the top-hit identity at each cutoff level. Dark and light lines near top of plot show the mean and standard deviation of top-hit identity to natural sequences of an alignment generated with no mask (complete convergence on all pixels). Dark and light lines near lower end of plot indicate the mean and standard deviation of top-hit identity to natural sequences of an alignment of sequences with only the conservation pattern (and no coupling).

FIG. 29A: cmr matrix of the natural SH3 alignment. The sequence alignment on which the cmr matrix is based is shown in FIGS. 46A-RR:

FIG. 29B: cmr matrix of the randomized alignment based on the natural SH3 alignment.

FIG. 29C: cmr matrix of artificial SH3 sequences created using a version of the optimization algorithm that lacks a mask, and thus converges on all matrix elements.

FIGS. 29D-I: cmr matrices of the artificial SH3 sequences created using a version of the optimization algorithm that includes a mask, where different significance cutoffs were used for each mask.

FIG. 30A: cmr matrix of the natural SH3 alignment.

FIG. 30B: difference matrix calculated between the cmr matrix of FIG. 30A and the cmr matrix shown in FIG. 29B.

FIGS. 30C-I: difference matrices, respectively, between the cmr matrix shown in FIG. 30A and those shown in FIGS. 29C-I.

FIG. 31: plot showing comparable values to those in FIG. 28 that were determined using an alignment of natural Dihydrofolate Reductase sequences. The alignment of the natural Dihydrofolate Reductase used is shown in FIGS. 47A-RRRR.

FIG. 32A: cmr matrix of the natural Dihydrofolate Reductase alignment.

FIG. 32B: cmr matrix of the randomized alignment based on the natural Dihydrofolate Reductase alignment.

FIGS. 32C-H: cmr matrices of the artificial Dihydrofolate Reductase sequences created using a version of the optimization algorithm that includes a mask, where different significance cutoffs were used for each mask.

FIG. 33A: cmr matrix of the natural Dihydrofolate Reductase alignment.

FIG. 33B: difference matrix calculated between the cmr matrix of FIG. 33A and the cmr matrix shown in FIG. 32B.

FIGS. 33C-H: difference matrices, respectively, between the cmr matrix shown in FIG. 33A and those shown in FIGS. 32C-H.

FIG. 34: plot showing comparable values to those in FIGS. 28 and 31 that were determined using an alignment of natural SH2 sequences. The alignment of the natural SH2 sequences used is shown in FIGS. 48A-PPPPP.

FIG. 35A: cmr matrix of the natural SH2 alignment.

FIGS. 35B-G: cmr matrices of the artificial SH2 sequences created using a version of the optimization algorithm that includes a mask, where different significance cutoffs were used for each mask.

FIG. 36A: cmr matrix of the natural SH2 alignment.

FIGS. 36B-G: difference matrices, respectively, between the cmr matrix shown in FIG. 36A and those shown in FIGS. 35B-G.

FIG. 37: Conservation pattern for alignment of fluorescent proteins. The fluorescent proteins used in this analysis are listed in FIGS. 49A-L.

FIG. 38: cmr matrix of C_{i,j}^{ev} values for the alignment of fluorescent proteins, where the C_{i,j}^{ev} values are represented on a color scale (right) from blue (0) to red (1.2).

FIG. 39: the clustered cmr matrix, with C_{i,j}^{ev} values shown on the color scale shown in FIG. 38.

FIG. 40: enlarged detail view of a portion of the FIG. 39 matrix, showing one network of co-evolving positions.

FIG. 41: enlarged detail view of a portion of the FIG. 36 matrix, showing another network of co-evolving positions.

FIG. 42: mapping the positions identified in FIGS. 40 and 41 on the structure 1 GFL (GFP from Aequorea Victoria).

FIGS. 43A-I: sequence alignment of natural WW domains to which FIGS. 2-5 pertain.

FIGS. 44A-C: sequence alignment of the natural WW domains used in one of the optimization algorithms described below to generate artificial domains and to make FIGS. 21, 22, 23, and 25.

FIGS. 45A-E: sequence alignment of natural PDZ domains to which an embodiment of the present methods was applied.

FIGS. 46A-RR: sequence alignment of natural SH3 domains. Sequences shown are SEQ ID NO. 172-670 (listed from top to bottom).

FIGS. 47A-RRRR: sequence alignment of natural Dihydrofolate Reductase sequences.

FIGS. 48A-PPPPP: sequence alignment of natural SH2 domains.

FIGS. 49A-L: sequence alignment of fluorescent proteins.

### DESCRIPTION OF ILLUSTRATIVE EMBODIMENTS

The terms “comprise” (and any form of comprise, such as “comprises” and “comprising”), “have” (and any form of have, such as “has” and “having”), “contain” (and any form of contain, such as “contains” and “containing”), and “include” (and any form of include, such as “includes” and “including”) are open-ended linking verbs. As a result, a method, a computer readable media or a device/system (e.g. a computer system, a circuit, or the like) that “comprises,” “has,” “contains,” or “includes” one or more steps or elements possesses those one or more steps or elements, but is not limited to possessing only those one or more steps or elements. Likewise, an element of a device that “comprises,” “has,” “contains,” or “includes” one or more features possesses those one or more features, but is not limited to possessing only those one or more features. The term “using” should be interpreted in the same way. Thus, and by way of example, a step in a that includes “using” certain information means that at least the recited information is used, but does not exclude the possibility that other, unrecited information can be used as well. Furthermore, something that is configured in a certain way must be configured in at least that way, but also may be configured in a way or ways that are not specified.

The terms “a” and “an” are defined as one or more than one unless this disclosure explicitly requires otherwise. The terms “substantially” and “about” are defined as at least close to (and includes) a given value or state (preferably within 10% of, more preferably within 1% of, and most preferably within 0.1% of). The terms “protein” and “polypeptide” are used interchangeably and refer to amino acid polymers; however, they are not limited to natural amino acids, and may also comprise modified amino acids (e.g., phosphorylated, glycosylated, or acetylated amino acids).

The techniques set forth below represent examples of ways in which the present methods may be performed. Those of ordinary skill in the art will recognize that other techniques may be used without departing from the scope of the claims.

The present methods may be implemented using a single computer or using a distributed computing environment.

The present computer systems may comprise one or more computers, such as those those connected by any suitable number of connection mediums (e.g., a local area network (LAN), a wide area network (WAN), or other computer networks, including but not limited to Ethernets, enterprise-wide computer networks, intranets and the Internet).

1.0 Testing an Alignment

The first step in some (but not all) embodiments of the present methods comprises testing the size and diversity of an alignment of a family of M biological sequences for size and diversity, each sequence having N positions, each position being occupied by one biological position element of a group of biological position elements. (In some embodiments of the present methods, no testing occurs.) Examples of suitable biological sequences include any that can be structurally aligned, whether through primary or tertiary structure, such as protein sequences and nucleic acid sequences. For protein sequences, the biological position elements are amino acids, and for nucleic acid sequences they are nucleic acids. In some versions of this step, the alignment used is the type known in the art as a multiple sequence alignment (MSA).

The alignment that is tested may reside as data on a computer system, such as in memory where the data has been loaded from a storage device, such as a disk drive, a USB drive, a CD, or the like. The data that represents the alignment may be organized in any suitable fashion (as may all the matrices discussed in this disclosure) that can be interpreted as having M rows (the biological sequences) and N columns (the biological sequence positions). For example, the data may be organized in look-up tables; or as a one-dimensional list of values that, by operation of an algorithm, is indexed as the elements in the alignment.

While alignment creation is not considered part of the present methods, those of ordinary skill in the art will recognize that there are many available techniques for creating alignments of biological sequences such as proteins. By way of example, Volume 266 of Methods in Enzymology (©1996) entitled “Computer Methods in Macromolecular Sequence Analysis” addresses the process of creating MSAs. For example, section III of this work, entitled “Multiple Alignment and Phylogenetic Trees,” addresses MSAs of biological sequences such as proteins and DNA.

Other works that address protein MSAs include “Gapped BLAST and PSI-BLAST: a new generation of protein database search programs” by Altschul et al., (1997); and “SCOP: a Structural Classification of Proteins database” by Hubbard et al., (1999). Works that address RNA MSAs include “The Ribonuclease P Database” by Brown (1999); “tRNAscan-SE: a program for improved detection of transfer RNA genes in genomic sequence” by Lowe et al., (1997); “Conservation of functional features of U6atac and U12 snRNAs between veterbrates and higher plants” by Shukla et al., (1999); and “The uRNA database” by Zwieb (1997).

Methods discussed in Volume 266 have been fundamental to a recognition of sequence homology as implemented in the BLAST® family of search tools, many of which—including versions of BLAST and PSI-BLAST (which also actually create alignments)—have existed in the art for several years; to the creation of phylogenetic trees for several years; and have been routine in the practice of molecular biology for guiding experimentation for several years.

Alignments such as MSAs (and, more specifically, protein MSAs) also may be created manually. Computer programs such as ClustalW (Thompson et al., 1994) also may be used to create biological sequence alignments such as protein MSAs.

The following specific steps may be performed on a Unix platform and used to create an alignment of biological sequences suitable for use with the present methods:

1) Choose a starting biological sequence (or set of biological sequences) that is a representative of the family. The sequence should be formatted as a fasta file—such as the following, called input.seq:

>gi|49176138|ref|NP_416237.3| 6-
phosphofructokinase II [Escherichia coli K12]

(SEQ ID NO: 161)

MVRIYTLTLAPSLDSATITPQIYPEGKLRCTAPVFEPGGGGINVARAIAH
LGGSATAIFPAGGATGEHLV
SLLADENVPVATVEAKDWTRQNLHVHVEASGEQYRFVMPGAALNEDEFRQ
LEEQVLEIESGAILVISGSL
PPGVKLEKLTQLISAAQKQGIRCIVDSSGEALSAALAIGNIELVKPNQKE
LSALVNRELTQPDDVRKAAQ
EIVNSGKAKRVVVSLGPQGALGVDSENCIQVVPPPVKSQSTVGAGDSMVG
AMTLKLAENASLEEMVRFGV
AAGSAATLNQGTRLCSHDDTQKIYAYLSR

2) Collect more representative biological sequences of the family using PSI-BLAST. PSI-BLAST finds a set of similar biological sequences and generates a profile to better represent the family. This profile is then used to search for more divergent sequences in an iterative process, as set forth in the following module:

```
blastpgp -i input.seq -o input.psi -j 50 -v 10000 -b 10000 -I T
blastpgp -i input.seq -o input.psi_6 -j 50 -v 10000 -b 10000 -I T -m 6
The arguments of blastpgp are:
-d Database [String]
-i Query File [File In] default = stdin
-o Output File for Alignment [File Out] Optional default = stdout
-j Maximum number of passes to use in multipass version [Integer]
default = 1
-v Number of one-line descriptions (V) [Integer]
  default = 500
-b Number of alignments to show (B) [Integer]
  default = 250
-I Show GI's in defines [T/F] default = F
-m alignment view options:
    6 = flat master-slave, no identities and blunt ends
```

The −j flag above dictates the maximum number of iterations to run, and is the main variable parameter in these commands. Often, sufficiently-large alignments are accessible with only a few rounds. Larger values tend to find more biological sequences, but risk including non-homologues. Choosing a value for the −j flag is somewhat heuristic. Values for the −v and −b flags are set arbitrarily large (so that they are not limiting).

3) Generate a list of gi numbers and escores from the PSI-BLAST runs: blast2escore<input.psi>input.escore

4) Generate an alignment of biological sequences, all with escores greater than 0.001:

escore2aln<input.escore input.psi_{—}6 0.001>input.psi_free

5) Delete redundancy and spaces in the alignment:

pretty_aln<input.psi_free>input.free

6) This procedure can be repeated, now using each of the biological sequences in input.free as the query for subsequent rounds of PSI-BLAST. This is called “transitive blast.” Afterwards, all of the biological sequences generated are combined into a single file: input_all.free.

7) Turn the alignment into a list of fasta formatted sequences:

format<input_all.free fasta>input.fasta

8) Generate an initial alignment, using PCMA (Pei et al., 2003):

pcma input.fasta

PCMA generates output in ClustalW format (.aln file), which is re-formatted to “free” format:

readprintali input.aln 10000>input2.free

9) Delete redundancy and spaces in the alignment:

pretty_aln<input2.free>input2.freep

10) Often, only a portion of the biological sequence is aligned with the query, leaving “jagged” ends in the alignment. The following module fills in the ends of biological sequences in the alignment, using biological sequence information from the database:

fill_ends<(database file) input2.freep>input3.free

11) Reformat to fasta, re-align, and fill the ends again:

format < input3.free fasta > input3.fasta
pcma input3.fasta
readprintali input3.aln > input4.free
fill_ends < (database file) input4.free > input5.free

12) Hand adjustments to the alignment may produce an even higher-quality alignment. Suitable hand adjustments can include the following:
A) 3D structure-based adjustment of the alignment: If some of the biological sequences in the alignment have known 3D structures, these can be used to modify the alignment. Structures may be aligned using their backbone atom coordinates—the biological sequence alignment is modified to agree with the structural alignment. There are software packages that facilitate this, such as Cn3D from NCBI. This has varying degrees of utility, depending on how many structures are available, and on how well they represent the sequence diversity in the alignment.
B) Secondary structure based adjustment of the alignment using known (or predicted) secondary structure for one (or several) members of the family. The following rules may be used:
   i) gaps are typically outside regions of secondary structure;
   ii) in the case of protein sequences, proline and glycine typically flank secondary structure elements;
   iii) in the case of protein sequences, hydrophobic amino acids are rarely in loops by themselves;
   iv) in the case of protein sequences, insert gaps into loops that flank beta sheets by splitting in half and moving to either side of the gap;
   v) in the case of protein sequences, surface-exposed beta strands usually have alternate hydrophobic/hydrophilic residues, and tend to contain beta-branched residues; and
   vi) in the case of protein sequences, alpha-helices tend to be amphipathic, having hydrophobic residues at positions i, i+3, i+4 and i+7. Helices tend to not have beta-branched amino acids.
C) In the case of protein sequences, sequence-based adjustment: residues with similar physical or chemical properties should be aligned with one another. Residues may belong to multiple “groups;” for instance, the group of small residues may comprise glycine, alanine and serine. But serine is also a potential H-bond donor, along with threonine. Threonine is a beta-branched amino acid, like valine and isoleucine. Other groups include amino acids with aromatic side-chains, charged residues, bulky residues, etc.

The alignment testing may be characterized in a broad respect as testing a “statistical coupling analysis criterion” of the alignment, which criterion may take the form of alignment diversity in one embodiment, and alignment size in another embodiment. Multiple criteria may be tested. Testing both such statistical coupling analysis criteria may be done to best ensure that the alignment is sufficiently large and diverse to accommodate the performance of some preferred embodiments of the present methods.

1.1 Testing the Diversity of the Alignment

The diversity testing may be accomplished in different ways. In general, the diversity test should expose non-diverse alignments, which are alignments that lack one or more positions that are occupied by biological position elements at a frequency that is close to the mean frequency with which those biological position elements exist at that position or positions over a larger number of sequences than exist in the alignment in question. The more positions in an alignment that are occupied by biological position elements at a rate that is close to some average frequency determined over a larger number of sequences than exist in the alignment, the more diverse the alignment is.

In a preferred embodiment, the alignment should be sufficiently diverse that, in the case of protein sequences, the frequencies of amino acids at some sites (which also may be referred to as “positions” in this disclosure) in the alignment are similar to their mean values in all sequences contained in the non-redundant database of protein sequences as of the October 1999 release. For proteins, those mean values are referred to in this disclosure as “baseline mean frequencies.” The baseline mean frequencies for protein sequences are, in alphabetical order of single-letter amino acid code (ACDEFGHIKLMNPQRSTVWY): [0.072658, 0.024692, 0.050007, 0.061087, 0.041774, 0.071589, 0.023392, 0.052691, 0.063923, 0.089093, 0.023150, 0.042931, 0.052228, 0.039871, 0.052012, 0.073087, 0.055606, 0.063321, 0.012720, 0.032955].

Testing for such diversity may be accomplished by determining (e.g., calculating) an average conservation energy value (e.g., ΔE_{i,x}^{stat} or, even more generally, the frequency of a given biological position element at a given position), where i represents a position and x represents an amino acid (or, for example, a nucleic acid in the case of nucleic acid sequences), for some of the least conserved positions in the alignment (e.g., 5% of the least conserved but highly occupied (e.g., ≧85%) positions in the alignment). The algorithm for calculating ΔE_{i,x}^{stat} is provided below. In the case of protein sequences, values for ΔE_{i,x}^{stat} that are close to zero represent frequencies of amino acids within the alignment that are close to their respective baseline mean frequencies.

A similar technique may be used to test the adequacy of the diversity of other biological sequences, such as nucleic acid sequences (e.g., DNA and RNA). The baseline mean frequencies for such biological sequences may be any suitable values that are known and that are based on more sequences than exist in the alignment in question. One example of such a baseline mean frequency is based on the collection of biological sequences that comprises the database of all known and unique members of all families of a given kind of biological sequence.

1.2 Testing the Size of the Alignment

The size testing of the alignment may be accomplished in different ways. In general, the alignment should be large enough that random elimination of sequences from the alignment does not change the biological position element frequencies at sites by more than a desired amount. The less change that occurs, the better.

One suitable criterion (others are possible) for an alignment that is sufficiently large is that if twenty-five percent of its sequences are eliminated randomly over many trials (e.g., 100 trials) and the average statistical conservation value (e.g., ΔE_{i,x}^{stat} or, even more generally, the frequency of a given biological position element at a given position) over the trials at the least conserved positions (e.g., the five least conserved sites on the alignment that also show at least 85 percent occupancy; another suitable measure is five percent of the sites in the alignment (starting with the least conserved) that show at least 85 percent occupancy) is within one standard deviation of the statistical conservation value (e.g., ΔE_{i,x}^{stat}) at each of those positions in the original alignment. Such an alignment may be said to be in a state of near statistical equilibrium. Such an alignment reflects near saturation mutagenesis through evolution, and is near stationary. In the case of protein sequences, amino acid distributions at sites of the alignment will show different magnitudes of conservation, reflecting the underlying evolutionary pressure.

Another suitable manner of testing the size of an alignment involves following the average statistical conservation value (e.g., ΔE_{i,x}^{stat}) at the least conserved sites (which require the most number of biological sequences to accurately represent) as a function of the random elimination of varying fractions of sequences from the alignment. For example, one may find the five least conserved sites on the alignment that also show at least 85 percent occupancy, and carry out the random elimination method embodied in the MATLAB file random_elim_dg.m provided below. Doing so returns the following data as well as a figure plotting the average ΔE_{i,x}^{stat} for these sites as a function of eliminating (e.g., randomly) various fractions of the initial alignment. Each data point represents the average of randomly eliminating a given fraction of the alignment a certain number of times, such as 100 or 10. The file random_elim_dg.m takes in: an alignment (A), given as biological sequences X having N positions, and returns: data_out, a matrix comprising the number of biological sequences remaining in the alignment upon elimination (column 1), the average ΔE_{i,x}^{stat} of the five selected sites for random elimination of that fraction of biological sequences (column 2), and the associated standard deviation (column 3). The “size” test may be described as testing the size of the alignment.

The MATLAB file, or module, random_elim_dg.m (which appears at the end of the description but before the claims under the general heading “COMPUTER PROGRAM LISTINGS”) is configured for protein sequences and represents one way to carry out the diversity and size tests described above.

2.0 Calculating Statistical Conservation Values

The next step in some embodiments of the present methods involves calculating a statistical conservation value for each biological position element in a pair of biological position elements at different positions in the alignment. One such statistical conservation value is ΔE_{i,x}^{stat}, explained below. In other embodiments of the present methods, a statistical conservation value, such as ΔE_{i,x}^{stat}, is calculated for more than one pair of biological positions elements at different positions in the alignment up to each biological position element at each position in the alignment.

Performance of this step may, when implemented via a computer system, involve loading the validated alignment into MATLAB for processing, using the following m-file, which is configured for protein sequences:

get_seqs.m

function [labels,parent_seqs]=get_seqs(filename)
% usage: [seqID, alignment]=get_seqs(filename)
% imports an alignment in .free format. In this format, an alignment is
% represented as follows: in the case of protein sequence alignment,
each line should
%contain a seqID, a tab character,
% the sequence comprised of the 20 amino-acids and a gap denoted by a
% period or a dash. Each line is separated by a paragraph mark.
[labels,parent_seqs]=(textread(filename,‘%s%s’));
labels=char(labels);
parent_seqs=char(parent_seqs);

As the get_seqs.m module shows, the alignment should be in “free” format—a text file with each line containing a sequence label followed by a tab character, the amino acid sequence (in the case of a protein sequence alignment), and a carriage return. Gaps are represented as ‘.’ or ‘-’. The get_seqs.m module returns the labels and the alignment separately.

A preferred embodiment of the present methods has been performed on the WW domain. The WW alignment that was built and validated contains 400 sequences and 87 positions. The get_seqs.m file was executed for the WW domain using the following command:

[labels,ww]=get_seqs(‘aln90’);

In the case of protein sequences, the alignment may be truncated to the protein sequence for which a structure is available. For example, sequence number 79 in the WW domain alignment that was built corresponds to the WW domain of Pin1 (PDB accession 1F8A). Truncation may be done manually, using the following MATLAB command (which is itself a complete program/module):

ww_trunc=ww(:,find(ww(79,:)˜‘−’));

The resulting alignment, ww_trunc, has 400 sequences and 37 positions. The truncation process may be characterized as truncating the validated alignment, or, more specifically, as truncating the validated alignment to biological sequences for which a structure is available.

Biological sequences that display high pairwise identities most likely arise from organisms or genes that have recently diverged. Their sequences may be similar as a simple result of this evolutionary history rather than of energetic constraints on the biological position elements. To minimize artifacts arising from clades of highly similar sequences, the alignment may be trimmed of biological sequences with a target pairwise identity, such as biological sequences with greater than 90 percent pairwise identity, meaning that no two sequences share greater than 90 percent of the same biological position elements at their respective positions. The trimming process may be characterized as eliminating biological sequences from the alignment that have a pairwise identity that meets a pairwise identity criteria.

The m-file alnid2.m, provided below and which is configured for protein sequences, can be used to generate an alignment in which no two sequences share greater than 90 percent identity:

alnid2.m

```
function [new_aln,idkeeplist,idx]=alnid2(aln,cutoff);
% accepts an alignment, and a cutoff (fractional identity) and outputs an
% alignment of sequences were every sequence is less than cutoff top hit
% identity to any other sequence
idkeeplist = ones(size(aln,1),1);
for i = 1:size(aln,1)−1
  if round(i/10)==i/10
% disp(i)
  end
  for j = i+1:size(aln,1);
    s1 = aln(i,:);
    s2 = aln(j,:);
    h = (s1˜=‘−’) | (s2˜=‘−’); % only avoid sites with double gaps
    f = sum( (s1˜=s2) & h ) / sum(h);
    f= 1−f;
idx(i,j) = f;idx(j,i)=f;
    if (f > cutoff)
      idkeeplist(i)=0;
      j = i+1;
    end
  end
end
new_aln = aln(find(idkeeplist),:);
The alnid2.m file can be executed using the following command:
      ww90 = alnid2(ww_trunc,0.9);
```

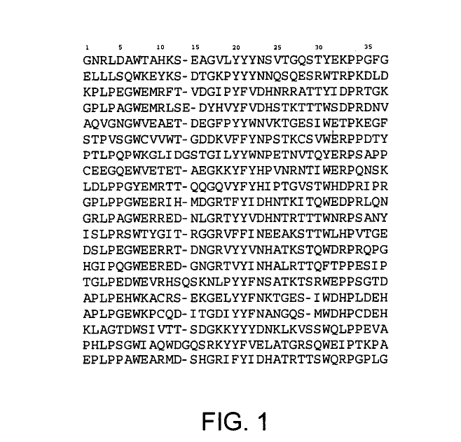

The resulting alignment, ww90, has 292 sequences and 37 positions. The first 20 sequences of this alignment, which were used in the examples provided in this disclosure (also characterized as the “working WW alignment”), are shown in FIG. 1. The highly conserved positions (7, 30 and 33) are highlighted in yellow.

Calculating the statistical conservation values for each biological position element in the pair of elements is a predicate to measuring the conserved co-variation of those elements. The parameter ΔE_{i,x}^{stat}, which is the conservation of a biological position element x at position i, is one suitable parameter for quantitatively representing sequence conservation, where iε{1, 2, . . . , N} in an alignment of N positions. For a protein, xε{ala, cys, asp, . . . , tyr}. For a nucleic acid sequence comprising DNA, xε{A, T, G, C}, and for a nucleic acid sequence comprising RNA, xε{A,U,G,C}.

The parameter ΔE_{i,x}^{stat} is a measure of the evolutionary conservation of a given biological position element at a given biological sequence position in the alignment; it is a measure of the degree to which the observed probability of a biological position element at one position differs from that observed randomly as represented by a baseline mean frequency for that biological position element (defined above). The m-file dg.m (which appears at the end of the description but before the claims under the general heading “COMPUTER PROGRAM LISTINGS”) executes the following steps (for protein sequences), which represent one manner of calculating the parameter ΔE_{i,x}^{stat} for each biological position element x at each position i in the alignment, although in a broad respect the calculation may be made for only two different elements at two different positions:

1) Calculate the frequency fᵢ^{x} of each biological position element x at each position iε{1, 2, . . . , N}:

$${f}_{i}^{x} = \frac{{m}_{i}^{x}}{M} ,$$

where mᵢ^{x} is the number of biological position elements x at position i in the alignment, and M is the total number of biological sequences in the alignment.

2) Calculate the probability of each biological position element x at each position iε{1, 2, . . . , N} by taking the binomial probability pix of observing fᵢ^{x} given a baseline mean frequency of that biological position element x. The total number of biological sequences in the alignment may be arbitrarily normalized to a value z to make the conservation parameter comparable between different alignments that differ in the number of biological sequences they contain:

$${P}_{i}^{x} = \frac{z !}{( {zf}_{i}^{x} ) ! ( z - {zf}_{i}^{x} ) !} {{p}_{x}^{{zf}_{i}^{x}} ( 1 - {p}_{x} )}^{z - {zf}_{i}^{x}} ,$$

where pₓ is a baseline mean frequency of biological position element x, and zfᵢ^{x} is the number of biological sequences having biological position element x in a normalized version of the alignment having z biological sequences. For proteins, these values are provided in the dg.m file below. The parameter z may be any suitable value; it is taken as 100 in the dg.m file below.

3) The conservation parameter ΔE_{i,x}^{stat} (which may also be characterized as a statistical parameter) of each biological position element x at each position i is calculated as the natural logarithm of the binomial probability Pᵢ^{x} relative to that observed overall in the alignment:

$$Δ {E}_{i , x}^{stat} = {γ}^{*} ln ( \frac{{P}_{i}^{x}}{{P}_{align}^{x}} ) ,$$

where P_{align}^{x} is the overall probability of biological position element x in the alignment, and γ* is an arbitrary unit of statistical energy.

These ΔE_{i,x}^{stat} at values may be arranged into a matrix of dimensions r×N, where r is the number of biological position elements in the group of biological position elements (e.g., 20 where the alignment contains naturally-occurring protein sequences and the group comprises all possible biological position elements). The group of biological position elements may be fashioned as desired. Thus, the group may comprise a subset of all amino acids in naturally-occurring protein sequences, such as aromatic residues (F, Y and W) or small residues (G, A and S). An r×N matrix contains all the ΔE^{stat} values for all biological position elements in the group at all positions in the alignment, and is referred to in this disclosure as the evolutionary conservation matrix. The following statement may be used to execute the m-file dg.m:

[DEmat,DEvec]=dg(ww90);

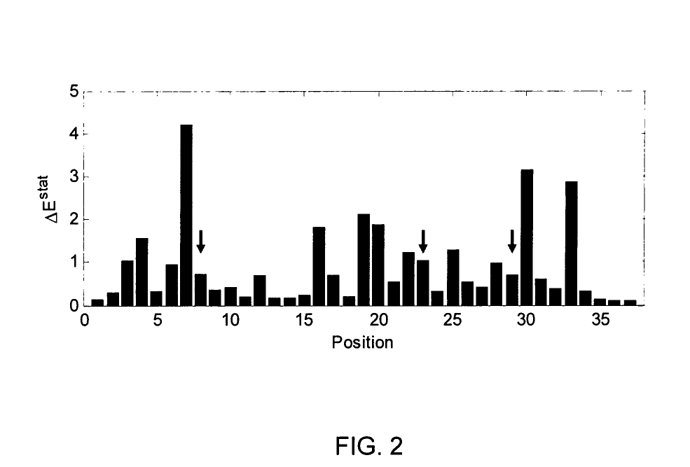

The dg.m file, which is configured for protein sequences, will generate two output variables: DEmat is the evolutionary conservation matrix. For the working WW alignment, the evolutionary conservation matrix has a size of 20 amino acids x 37 positions with elements of ΔE_{i,x}^{stat}. The dg.m file also returns DEvec, in which the evolutionary conservation matrix is reduced to a vector of size N positions (for the working WW alignment, N=37) by taking the magnitude of ΔE_{i,x}^{stat} values along the amino acid dimension. This vector can be displayed as a bar chart of ΔE^{stat} values, as shown in FIG. 2.

3.0 Measuring Conserved Co-Variation of at Least Two Elements

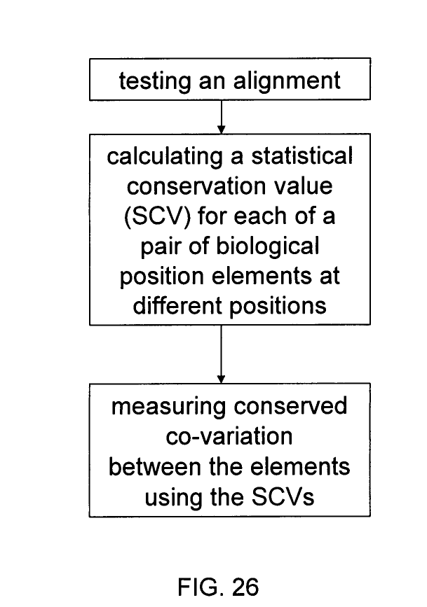

The next step in some embodiments of the present methods involves measuring the conserved co-variation of the two biological position elements for which the statistical conservation values were calculated (see FIG. 26). The measuring may take place with respect to any two desired biological position elements at different positions in the alignment, up to all pairs of elements whose member elements are at different positions. The measuring may be characterized as calculating the conserved co-variation of the elements or the conserved co-evolution of the elements. The process of measuring conserved co-variation between biological position elements at two different positions also may be more broadly characterized as measuring the statistical coupling of two positions in the alignment to each other.

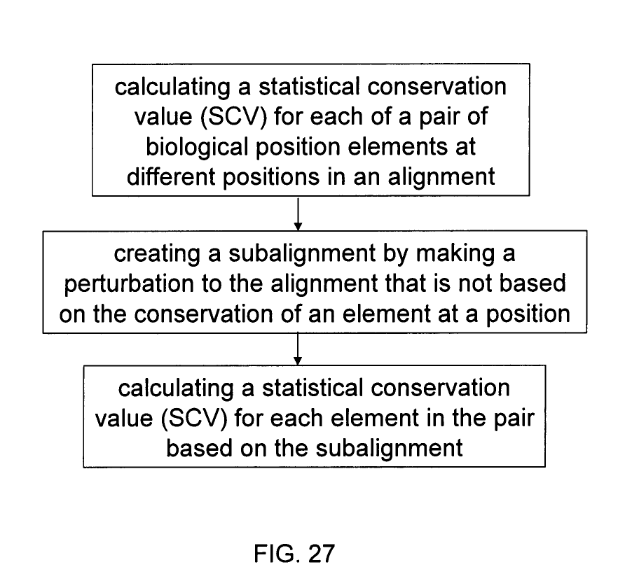

In a broad respect, one way to measure the conserved co-variation of a pair of biological position elements at different sites in the alignment includes making a perturbation to the alignment that is independent of the conservation of any particular sequence position or, more specifically, any particular biological position element at a particular position (see FIG. 27); and measuring the resulting change in conservation of the target biological sequences. Multiple such perturbations and measurements may be performed consistent with some embodiments of the present methods.

As a result of making such a “conservation-independent” perturbation, it is necessarily possible to attain a conserved co-variation score for any pair of biological position elements at any pair of sequence positions in an alignment. The same is not true when the perturbation style of Suel et al. is used, which perturbations are based on the conservation of a given biological position element at a given position.

In another broad respect, another way to measure the conserved co-variation of a pair of biological position elements at different sites in the alignment includes making a series of sufficiently small perturbations to the alignment and measuring the resulting change in conservation of the target biological sequences over the series of perturbations. The number of perturbations made may be related to the number of biological sequences in the alignment; thus, the number of perturbations made may, in different embodiments, include a number of perturbations equal to one percent up to an infinitely great percentage of the number of sequences in the alignment. The sequence or sequences eliminated in a given perturbation may be chosen randomly (e.g., using a random number generator).

In a more specific respect, the conserved co-variation of a pair of biological position elements at different positions in an alignment may be measured by carrying out one or more perturbations (e.g., of the type described above), determining the resulting difference in conservation of those elements between the parent alignment and perturbed (or sub-) alignment, and determining the similarity of the change in conservation in terms of direction and magnitude.

One way to determine a difference in conservation of a given biological position element at a given position comprises calculating a statistical difference parameter, such as ΔΔE_{i,x}^{stat} (described below). This parameter may be characterized as reflecting the change in the conservation (e.g., the ΔE_{i,x}^{stat} values) for a given biological position element x at a given position i after a perturbation to the alignment (e.g., the working WW alignment). One way to determine the similarity of the change in conservation between the biological position elements in a given pair of biological position elements x and y at two different positions iε{1, 2, . . . , N} and jε{1, 2, . . . , N} in the alignment in terms of direction and magnitude is to use a parameter such as C_{i,x,j,y}^{ev} (described below; the “ev” being shorthand for “evolution”). If the C_{i,x,j,y}^{ev} parameter is used and the alignment contains, for example, proteins sequences, then xε{ala, cys, asp, . . . , tyr} and yε{ala, cys, asp, . . . , tyr}.

Although different processes may be used (which may involve perturbations of different sizes and different numbers of perturbations), the preferred procedure for measuring the conserved co-variation of two biological position elements at two different positions (up to all pairs of elements at different positions) involves a leave-one-out process of perturbing the alignment. In the preferred process, each sequence is sequentially eliminated from the alignment, and the change in evolutionary conservation of a given biological position element x at a given position i for one sequence elimination is recorded in a “perturbation energy” value called ΔΔE_{i,x}^{stat}:

$$ΔΔ {E}_{i , x , δ m}^{stat} = ( Δ {E}_{i , x}^{stat} - Δ {E}_{i , x \vert δ m}^{stat} ) * \frac{M}{z} ,$$

where δm signifies the perturbation (e.g., the elimination of one sequence from the alignment in the preferred process), ΔE_{i,x}^{stat} is the conservation of biological position element x at position i, and ΔE_{i,x|δm}^{stat} is the conservation of biological position element x at position i given the perturbation. The ΔE^{stat} values are calculated as described above, and is a weighting factor that normalizes the perturbation energy for alignments of different size. As noted above, M/z is the total number of sequences in the alignment and z is an arbitrary normalization of alignment size that may be taken as 100, as described above.

This leave-one-out process may yield a vector of ΔΔE^{stat} values (characterizable as a “perturbation vector”), \vec{ΔΔE}_{i,x}^{stat}, for each biological position element x of interest at each position i of interest:

$${\vec{Δ Δ E}}_{i , x}^{stat} = \{ ΔΔ {E}_{i , x , δ m 1}^{stat} , ΔΔ {E}_{i , x , δ m 2}^{stat} , … , ΔΔ {E}_{i , x , δ mM}^{stat} \} .$$

This leave-one-out perturbation and co-variation measurement process is implemented in the m-file stat_fluc.m (which is configured for proteins and which appears at the end of the description but before the claims under the general heading “COMPUTER PROGRAM LISTINGS”), and may be executed using the following command:

perturbation_matrix=stat_fluc(ww90);

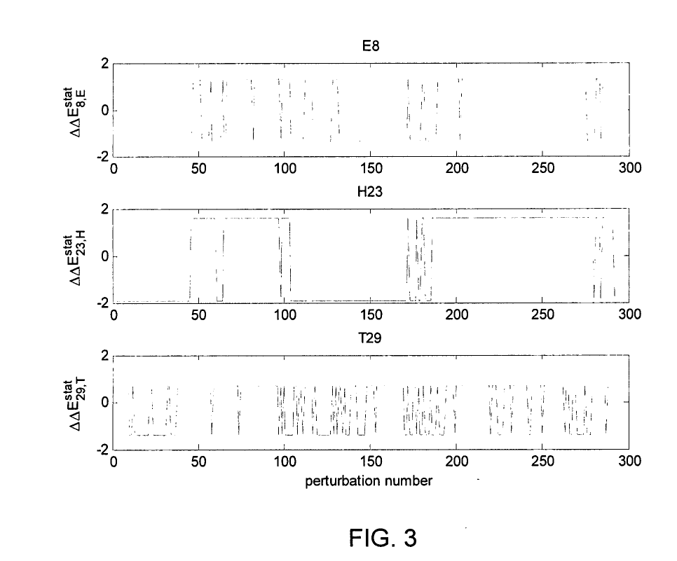

The stat_fluc.m file returns a set of values designated perturbation_matrix, which may be characterized as a matrix of size r biological position elements by N positions by M perturbations (for the WW alignment, 20 amino acids×37 positions×292 sequences) with elements of ΔΔE_{i,x}^{stat}. For the working WW alignment, there are 740 (20×37) \vec{ΔΔE}_{i,x}^{stat} perturbation vectors corresponding to each of the 20 amino acids at each of the 37 positions, where the process is applied, as it is in stat_fluc.m, to every pair of amino acids at different positions in the working WW alignment. Three such perturbation vectors are shown graphically in FIG. 3.

Each perturbation contributing to the vectors shown in FIG. 3 has one of two results. If the eliminated sequence includes residue x at position i (an E at position 8, for example) then the frequency of that residue decreases, causing a decrease in ΔE_{i,x}^{stat} and a positive ΔΔE_{i,x,δm}^{stat} value. Alternatively, if the eliminated sequence does not include residue x at position i, then the frequency of x increases and ΔΔE_{i,x,δm}^{stat} has a positive value. The similar magnitudes of the fluctuation upon perturbation for each of the three examples is a result of the similar conservation values for each of these sites.

The single-sequence eliminations from the working WW alignment represent subtle perturbations of the statistical structure of the working WW alignment in which sites that are not evolutionarily coupled are expected to show independent patterns of \vec{ΔΔE}ₛₜₐₜ fluctuations, and sites that are evolutionarily coupled are expected to show some mutual dependence in their fluctuations. To measure this evolutionary coupling for a pair of biological position elements x and y at a pair of positions i and j, the inner (or dot) product of the perturbation vectors \vec{ΔΔE}_{i,x}^{stat} and \vec{ΔΔE}_{j,y}^{stat} may be taken to yield the parameter C_{i,x,j,y}^{ev}:

$$\begin{matrix}{C}_{i , x , j , y}^{ev} = {\vec{Δ Δ E}}_{i , x}^{stat} \cdot {\vec{Δ Δ E}}_{j , y}^{stat} \\ = \vert {\vec{Δ Δ E}}_{i , x}^{stat} \vert \vert {\vec{Δ Δ E}}_{j , y}^{stat} \vert cos θ ,\end{matrix}$$

where θ is the angle between the two perturbation vectors in the M-dimensional space of all the perturbation trials. When the leave-one-out technique described above involves the measurement of conserved co-variation for multiple (up to all) pairs of biological position elements at different positions in the alignment, these C_{i,x,j,y}^{ev} values may be arranged into a dataset that may be characterized as a matrix having a size (or dimensions) T×T×u×u, where u is the number of biological position elements being compared at T different positions. In a preferred embodiment, T comprises N (the total number of biological sequences in the alignment) and u comprises r (the number of biological position elements in the group), such that the matrix has a size N×N×r×r. Such a matrix is referred to within this disclosure as a type of statistical coupling matrix (SCM), and its elements may be organized in any suitable fashion, such as in a look-up table of values or as a list of one-dimensional values that may be acted upon by a suitable algorithm to form the matrix.

The m-file global_sca.m, which appears below, is an example of a program configured to calculate the dot product of every pair of perturbation vectors that can be calculated after applying the leave-one-out technique to an alignment of protein sequences, such as the working WW alignment:

global_sca.m

```
function
[coupling_matrix_aa,coupling_matrix_res]=global_sca(randpert_mat);
[numaa, numpos, numtrials]=size(randpert_mat);
% amino_acids=(‘ACDEFGHIKLMNPQRSTVWY’);
coupling_matrix_aa = zeros(numpos, numpos, 20, 20);
coupling_matrix_res = zeros(numpos,numpos);
for m = 1:numpos
  site1 = squeeze(randpert_mat(:,m,:));
  for n = m:numpos
    site2 = squeeze(randpert_mat(:,n,:));
    coupling_matrix_aa(m,n,:,:) = site1*site2’;
    coupling_matrix_aa(n,m,:,:) =
squeeze(coupling_matrix_aa(m,n,:,:))’;
  end
end
coupling_matrix_aa=coupling_matrix_aa./numtrials;
for m=1:numpos
  for n=1:numpos
    coupling_matrix_res(m,n) =
sqrt(sum(sum(coupling_matrix_aa(m,n,:,:).\hat{ }2)));
  end
end
The global_sca.m file may be executed using the following command:
      [coupling_matrix_aa,coupling_matrix_res]=-
      global_sca(perturbation_matrix);
```

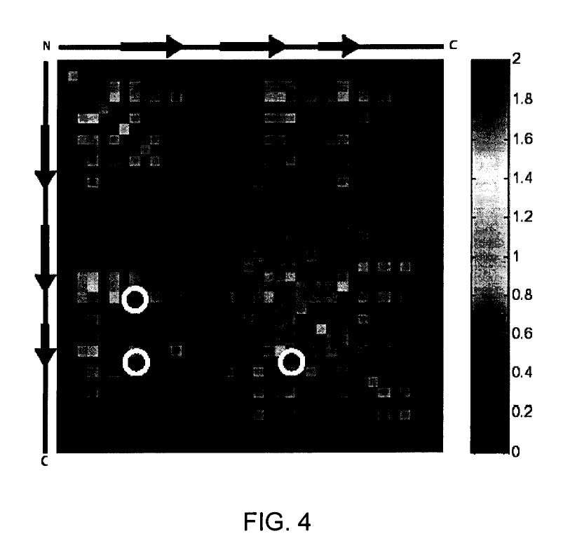

The file global_sca.m returns the variable coupling matrix_aa (“cma”), which is one SCM. For the working WW alignment, this matrix is of size 37 positions×37 positions×20 amino acids x 20 amino acids. As shown in FIG. 3, residues E8 and H23 have a similar pattern of fluctuation. The C_{i,x,j,y}^{ev} for this pair is 1.84. Residue T29 has an unrelated pattern of fluctuation (see FIG. 3), and a correspondingly low coupling score with E8 (0.33) and with H23 (0.37). The file global_sca.m also returns the variable coupling matrix_res (“cmr”), which is a reduced matrix (and another version of an SCM) of, in this case, N positions by N positions (for the working WW alignment, 37 positions×37 positions) that is generated by taking the magnitude of the C^{ev} values in the cma SCM along both amino acid dimensions, yielding a group of C_{i,j}^{ev} values. Both the C_{i,x,j,y}^{ev} values and the C_{i,j}^{ev} values may be characterized as C^{ev} values in this disclosure. The cmr matrix for the working WW alignment is the matrix shown in FIG. 4. This matrix is both square and symmetric. As a result of the symmetry, the conserved co-variation scores embodied by the C_{i,j}^{ev} values are symmetric with respect to each pair of scores. Thus, C_{i,j}^{ev}=C_{j,i}^{ev}. In the cma matrix, the C_{i,x,j,y}^{ev} values are symmetric, such that C_{i,x,j,y}^{ev}=C_{j,y,i,x}. Thus, in some embodiments of the present methods, measuring conserved co-variation between the biological position elements in a pair located at different positions using the statistical conservation values for those elements yields conserved co-variation scores for those elements that are symmetric.

In one respect, the C_{i,x,j,y}^{ev} parameter may be characterized as a measure of the statistical correlation between the occurrences of a pair of biological position elements at a pair of sequence positions, weighted by the conservation of each biological position element. In one respect, the C_{i,j}^{ev} parameter may be characterized as a measure of the statistical correlation between the occurrences of a pair of sequence positions, weighted by the conservation of each sequence position. As will be understood by those of ordinary skill in the art, a position in a given alignment may be characterized as conserved (at least to some degree) where the frequency of a biological position element at that position deviates from a baseline mean frequency. In general, a C^{ev} value may be characterized as a type of conserved co-variation or conserved co-evolution score.

4.0 Analysis of the Information in a Statistical Coupling Matrix

The information in a given SCM having more than 2 dimensions may be more easily visualized by taking the magnitude of the correlated conservation score (e.g., the C^{ev} value) along one or more of the dimensions of the SCM. For example, the information in the 4-dimensional cma SCM described above may be reduced in size by taking the magnitude of the C^{ev} scores along the two amino acid dimensions, resulting in the cmr SCM shown in FIG. 4. In the example shown in FIG. 4, high values in the cmr SCM indicate co-evolution between two positions in the alignment (e.g., the working WW alignment).

4.1 Clustering of Co-Evolving Pairs of Biological Sequences

Another step in some embodiments of the present methods comprises identifying multiple pairs (also characterizable as groups or clusters) of biological sequence positions that co-evolve, or co-variate, together. Such an identification involves at least two locations on, for example, the SCM shown in FIG. 4, because a given location on the SCM shown in FIG. 4 is an example of a single pair of positions that co-evolve together. One way to make such an identification includes the use of a clustering algorithm, such as an algorithm configured for two-dimensional hierarchical clustering, which can involve techniques developed for recognizing pattern similarities in large datasets. Such techniques were applied to a predecessor version of an aspect of the present methods in Suel et al.

As discussed below in section 4.2, clusters of evolutionarily-coupled positions may then be mapped on the 3D structure of the biological sequence in question in order (1) to determine functionally important biological sequence positions (e.g., in proteins), and (2) to identify potential communication mechanisms between functional positions.

One way to perform two-dimensional heirarchical clustering of a given SCM, such as the cmr matrix, includes three steps that are codified in the m-file rr_cluster_{—}2.m (provided below), using the following command:

[p_pos,1_pos,sort_pos,sorted]=rr_cluster_{—}2(cmr,1:37,2,rama_map,0);

1) First, a set of distances between each pair of positions within the SCM is calculated. Each position i is represented by the vector of C_{i,j}^{ev} values for all positions j; in other words, each row (and column) of the SCM (e.g., the cmr matrix in FIG. 4) represents a position. The m-file rr_cluster_{—}2.m uses the MATLAB function pdist.m to calculate distances between positions; more specifically, it uses the city-block distance metric, which is known to those of ordinary skill in this art.

2) The distances from pdist are used to generate a linkage map, using the MATLAB function linkage.m. In the m-file rr_cluster_{—}2.m, linkage is executed using the “complete” or “furthest neighbor” algorithm, which is known to those of ordinary skill in this art. The linkage is depicted using a dendrogram.

3) The rows and columns of the SCM are then re-sorted according to the clustering pattern indicated by the linkage output.

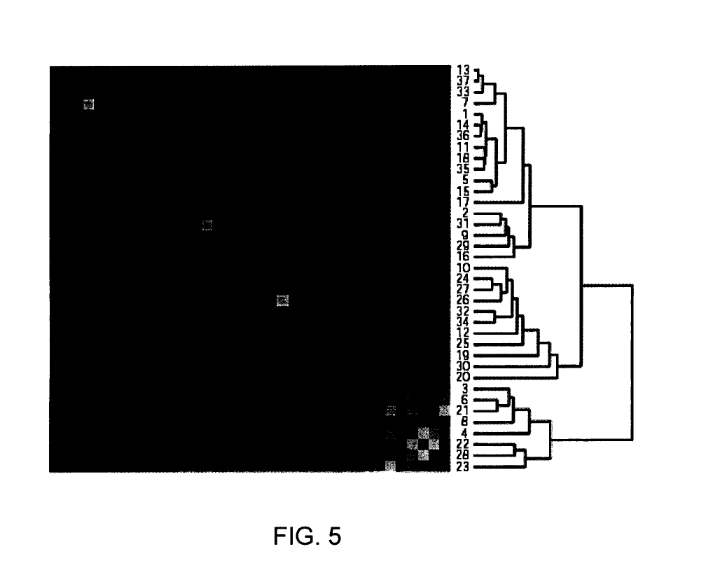

The m-file rr_cluster_{—}2.m output comprises p_pos, which is the distance data from pdist; 1_pos, which is the linkage data; sort_pos, which is the order of positions in the linkage map; sorted, which is, in this example, the cmr matrix re-ordered by sort_pos. The resulting matrix and dendrogram for the working WW alignment is shown in FIG. 5. The m-file rr_cluster_{—}2.m appears below:

rr_cluster_2.m

```
function
[p_pos,l_pos,sort_pos,sorted]=rr_cluster_2-
(mat,pos_labels,max_scale,colormap_in,raw_ or_not);
% The program takes in SC matrix (mat), the position labels, a max_scale
% for linear mapping of the color map to DDEstat values,
% the colormap, and a flag (raw_or_not) that determines whether an
% unclustered version of the matrix is kicked out as well. Returns the
% distance matrices for positions (pdist output, p_pos), the clusters for
%positions (linkage output, l_pos), the sorted indices
% for positions (sort_pos), and figures of the clustered matrix, the
% position dendrogram, and if you choose, the unclustered matrix.
[x,y]=size(mat);
p_pos=pdist(mat,‘ci’);
if x>2
  l_pos=linkage(p_pos,‘co’);
  [a,b,sort_pos]=dend_rr(l_pos,pos_labels,1);
else
  sort_pos=[1 2];
end
sorted=mat(sort_pos,sort_pos);
if colormap_in==0
  map=‘jet’;
else
  map=colormap_in;
end
if max_scale==0
  figure;imshow(sorted,[0
max(max(mat)’)],‘InitialMagnification’,‘fit’);colormap-
(map);brighten(0);colorbar
else
  figure;imshow(sorted,[0
max_scale],‘InitialMagnification’,‘fit’);colormap-
(map);brighten(0);colorbar
end
if raw_or_not==1
  if max_scale==0
    figure;imshow(mat,[0
max(max(mat)’)],‘InitialMagnification’,‘fit’);colormap-
(map);brighten(0);colorbar
  else
    figure;imshow(mat,[0
max_scale],‘InitialMagnification’,‘fit’);colormap-
(map);brighten(0);colorbar
  end
end
```

Applied to the working WW alignment, the clustering achieved using rr_cluster_{—}2.m shows few pairs of coupled sequence positions (or matrix elements) that are strongly coupled; most pairs of sequence positions show low C^{ev} values. The clustering also reveals that matrix elements with high C^{ev} values exhibit a highly redundant pattern of coupling, and therefore group into a single, well-resolved cluster.

4.2 Independent Components Analysis

As an alternative to clustering as a way to identifying multiple pairs of biological sequence positions that co-evolve, or co-variate, together, one may use Independent Components Analysis (ICA) and the related techniques of Eigenvalue Analysis or Principle Components Analysis. ICA is a statistical and computational technique for revealing hidden factors that underlie sets of random variables, measurements, or signals. ICA defines a generative model for the observed multivariate data, which is typically given as a large database of samples. In the model, the data variables are assumed to be linear or nonlinear mixtures of some unknown latent variables, and the mixing system is also unknown. The latent variables are assumed nongaussian and mutually independent, and they are called the independent components of the observed data. These independent components, also called sources or factors, can be found by ICA. In this disclosure, the independent components comprise groups of biological sequence positions that co-evolve, or co-variate, together.

Techniques for performing ICA on a given SCM, such as the cmr matrix, include those disclosed in U.S. Pat. No. 6,936,012, which is incorporated by reference. An ICA algorithm embodied in the FastICA package, a free (GPL) MATLAB program available on the Internet, may be used. This package implements the fast fixed-point algorithm for independent component analysis and projection pursuit. The newest version of FastICA is version 2.5, published on Oct. 19, 2005.

4.3. Mapping Clusters onto a 3D Structure

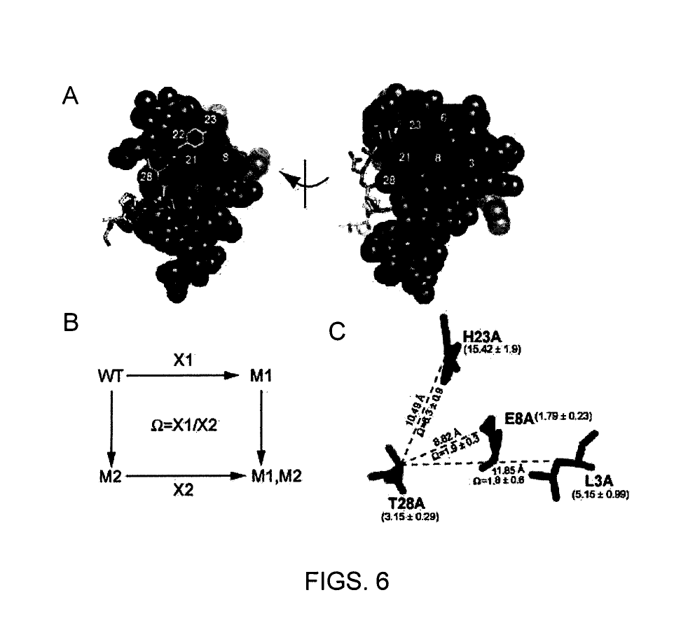

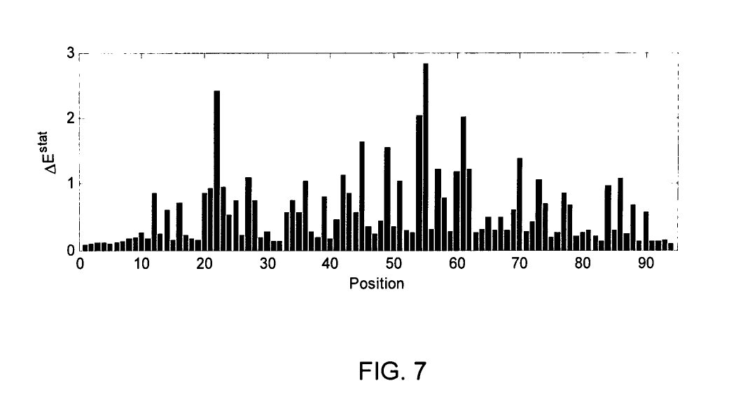

Another step in some embodiments of the present methods comprises mapping clustered biological sequence positions, or groups of biological sequence positions identified using ICA, Principle Components Analysis or Eigenvalue Analysis, onto a 3D structure of a member of the family of biological sequences. The result of such mapping as applied to the working WW alignment is shown in FIG. 6A.

FIG. 6A shows that mapping the cluster of coupled biological sequence positions onto the 3D structure of a WW domain (Pin1, PDB accession 1F8A) produces an unexpectedly distributed picture of binding specificity in that WW domain. In this regard, the mapping is of the biological positions elements present at a given position in a given domain. Rather than being restricted to the ligand binding surface, the eight networked residues are organized into a physically contiguous network linking the primary specificity determining pocket (the residues at positions 21, 23, and 28) with residues on the opposite side (at positions 3 and 4) through a few intervening residues (at positions 6, 8, and 22). The co-evolution of these positions predicts that (1) some residues act at long-range in mediating peptide binding and (2) the networked amino acids act cooperatively in determining the binding free energy.

Previous mutagenesis studies already suggest that network residues at or close to the peptide binding surface (see positions 8, 21, 23, and 28) mediate binding specificity (Chen et al., 1997; Espanel and Sudol, 1999; Kasanov et al., 2001; Toepert et al., 2001), and structural work in the dystrophin WW domain provides a mechanistic understanding for the contribution of these residues in group I domains (Huang et al., 2000). To test more of the network for contribution to peptide binding, and to test the prediction of cooperative action, thermodynamic double mutant cycle analysis (Carter et al., 1984; Hidalgo and MacKinnon, 1995) was carried out to measure the energetic coupling between mutations at binding-site position 28 and positions 3, 8, and 23 in the Nedd4.3 WW domain. In the mutant cycle method, the effect of one mutation on the equilibrium dissociation constant for peptide binding is measured in two conditions: (1) the wild-type background

$$( X 1 = \frac{{K}_{d}^{M 1}}{{K}_{d}^{WT}} ) ,$$

and (2) in the background of a second mutation

$$( X 2 = \frac{{K}_{d}^{M 1 , M 2}}{{K}_{d}^{M 2}} )$$

(FIG. 6B). The ratio of these effects gives the coupling parameter Ω, a measure of the degree of interaction between the two mutations. If the two mutations are thermodynamically independent, the effect of the first mutation is the same in conditions 1 and 2, and Ω=1. If Ω≠1, then the two mutations act cooperatively.

Consistent with earlier studies (Kasanov et al., 2001; Huang et al., 2000), the E8A, H23A, and T28A mutations all affected binding of a PPxY-containing peptide (FIG. 6C). However, L3A also had a significant effect (5.15+/−0.99 fold) though located on the opposite surface from the peptide binding site. In addition, mutant cycle analyses for the T28A mutation with each of the three other mutations show Ω values that significantly differ from unity (FIG. 6C). Specifically, the effects of mutations at 3, 8, and 23 are either diminished (L3A and H23A) or abrogated (E8A) in the background of T28A. Thus, T28A is thermodynamically coupled to mutations at 3, 8, and 23. These results support the model that a distributed and cooperative network of residues predicted by use of the preferred embodiment (described above) of the present methods contributes to peptide recognition in the WW domain.

Statistical Coupling Analysis of the PDZ Domain

The process described in sections 1.0 through 3.0 above may be characterized as a type of statistical coupling analysis (SCA) that can be applied to a family of biological seqeunces. In addition to applying it to the WW domain, as detailed above, it has also been applied to the PDZ domain. PDZ domains are a family of protein interaction motifs that bind to the C-termini of their targets. The SCA-based analysis of the PDZ family that was performed (which included the validation of the alignment, the truncation of the alignment, and the trimming of the alignment) identified amino acids at different PDZ positions that are important for ligand binding and activity.

The PDZ alignment of 240 sequences and 129 positions that were validated were read into the m-file get_seqs.m using the following command:

[lab,aln]=get_seqs(‘pdz.free’);

The get_seqs.m file follows:

get_seqs.m

function [labels,parent_seqs]=get_seqs(filename)
% imports an alignment in .free format. In this format, an alignment is
% represnted as follows: each line should contain a seqID, a tab character,
% the sequence comprised of the 20 amino-acids and a gap denoted by a
% period or a dash. Each line is separated by a paragraph mark.
[labels,parent_seqs]=(textread(filename,‘%s%s’));
labels=char(labels);
parent_seqs=char(parent_seqs);

The alignment had gaps, and was visualized on the structure of PSD95 (PDB accession 1BE9). The alignment to the sequence shown in this structure was truncated to remove gaps, so that all positions in the alignment had a corresponding amino acid in the PDB file. Sequence number 79 in the alignment corresponded to the 1BE9 structure, so the following command (which comprises the program) was used:

taln=aln(:,find(aln(79,:)˜=‘−’));

The output taln is the truncated alignment with a size of 240 sequences×94 positions. Calculating the conservation pattern of the truncated alignment using dg.m provided above revealed that many positions have low conservation, and therefore the alignment has reached statistical equilibrium (see FIG. 7). The following command was used to execute the dg.m file provided above:

[DEmat,DEvec]=dg(taln);

As before, DEvec is the vector of ΔE^{stat} values generated by taking the magnitude of the ΔE_{i,x}^{stat} in DEmat. Plotting DEvec gives the bar chart in FIG. 7.

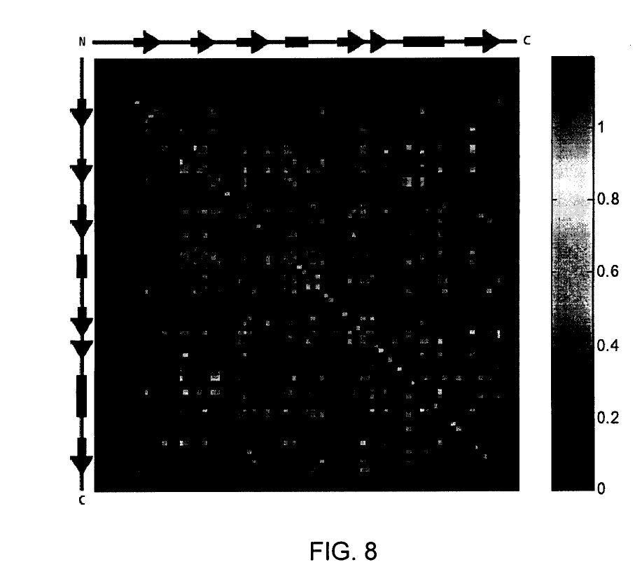

A cmr matrix like the one created for the WW domain was created for the PDZ domain, as shown in FIG. 8. As with the WW domain, the cmr matrix (one type of SCM) for the PDZ family shows sparse evolutionarily-coupled positions in the alignment (see FIG. 8). The following commands were used to the execute the stat_fluc.m and global_sca.m files for the PDZ alignment that was used:

perturbation_matrix=stat_fluc(at);
[coupling_matrix_aa,coupling_matrix_res]=global_sca-
(perturbation_matrix);

As before, the cmr matrix contained C_{i,x,j,y}^{ev} values that were reduced along both amino acid dimensions to give a series of C_{i,j}^{ev} values for all positions i and j.

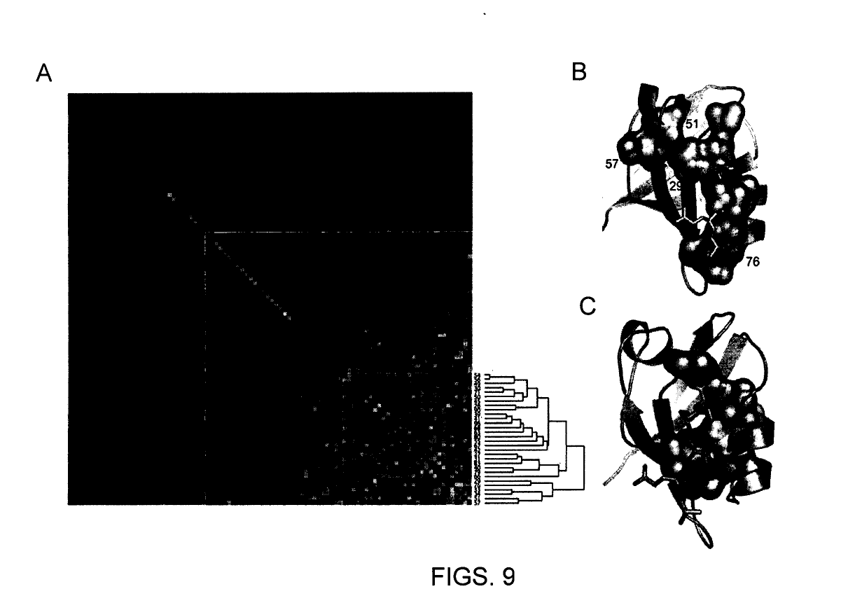

The clustering technique described above in section 4.1 was applied to the cmr matrix shown in FIG. 8 to produce the matrix shown in FIG. 9A, which comprises a more detailed version of a SCA. That clustering reveals a small cluster of co-evolving positions (see FIG. 9A) that, when mapped on a 3D structure of the PDZ domain as shown in FIGS. 9B and 9C using the residues at those positions of the depicted domain, form a single continuous unit that involves residues in the peptide binding site, the core, and the back surface of the protein. In this case, three rounds of hierarchical clustering were applied (the ultimate result of which is shown in FIG. 9A), each time excluding the main cluster with the weakest C^{ev} signals. Such iterative clustering is described, for example, in Suel et al.

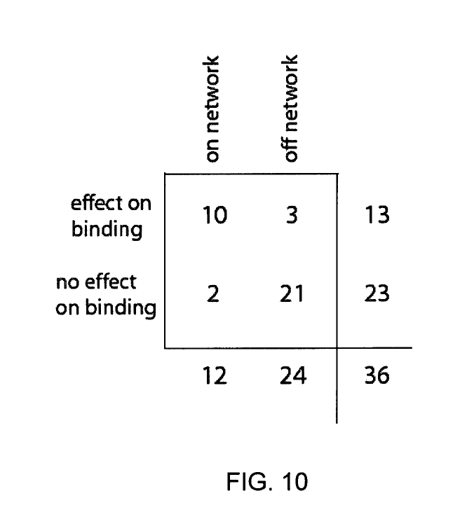

The importance of the coupled residues identified above are supported by a large-scale mutagenesis study in the Erbin PDZ domain. The study demonstrated that mutations of most of the highly-coupled residues identified above caused a significant effect on peptide binding activity (Skelton et al., 2003). In the Skelton study, a total of 36 conserved positions within the PDZ alignment were mutated to alanine, and tested for their effect on peptide binding. Of these 36, twelve of the residues were identified as being part of a coupled network using the clustering technique described above that produced the FIG. 9A results. Skelton showed that mutation of 10 of these 12 residues had an effect on the binding activity of the PDZ domain (see FIG. 10). In contrast, only 3 of the remaining 24 residues (not identified as being part of coupled network in FIG. 9A) had an effect on PDZ domain activity. This comparison demonstrates that the embodiment of SCA utilized as described above for the PDZ domain is more effective at correctly identifying residues important for protein domain folding and function than standard methods using only MSA data (p=0.00006 by Fisher Exact Test).

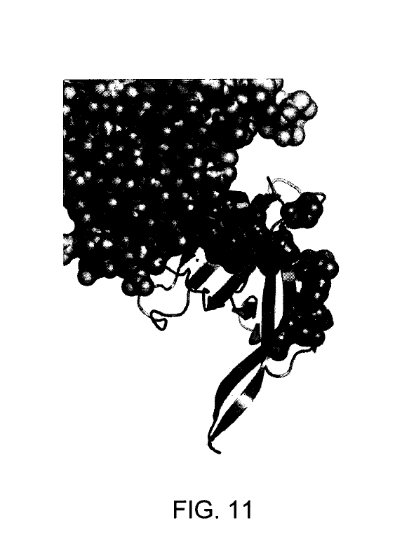

While the work in the Erbin PDZ domain provided an extensive mutagenesis-based test that confirms the predictive power of at least one embodiment of SCA, the study focused on the residues spatially close to the peptide binding site. Interestingly, SCA may also predict that some residues located far from the peptide binding site are also energetically coupled to ligand binding. Garrard et al. (2003) provides important evidence that supports predictions of long range biologically relevant interactions. These authors show that in the PDZ domain of Par6, a protein involved in epithelial tight junction formation, evolutionarily-coupled residues on the spatially distant helix αA comprise a binding site for an allosteric regulator known as CDC42 (FIG. 11). Furthermore, a mutation in the co-evolving network disrupts the allosteric regulation (Peterson et al., 2004). These data further demonstrate that SCA-based predictions may be used to identify structurally non-intuitive long-range allosteric mechanisms in proteins.

Statistical Coupling Analysis of the Fluorescent Protein Family

The version of SCA performed on the PDZ domain as described above was also performed on an alignment of 93 naturally occurring fluorescent proteins with no greater than 95% top-hit identity to each other. The discussion presented below pertains to positions in the alignment that are represented in the structure 1 GFL (GFP from Aequorea Victoria). The SCA performed included the validation of the alignment, the truncation of the alignment, and the trimming of the alignment.

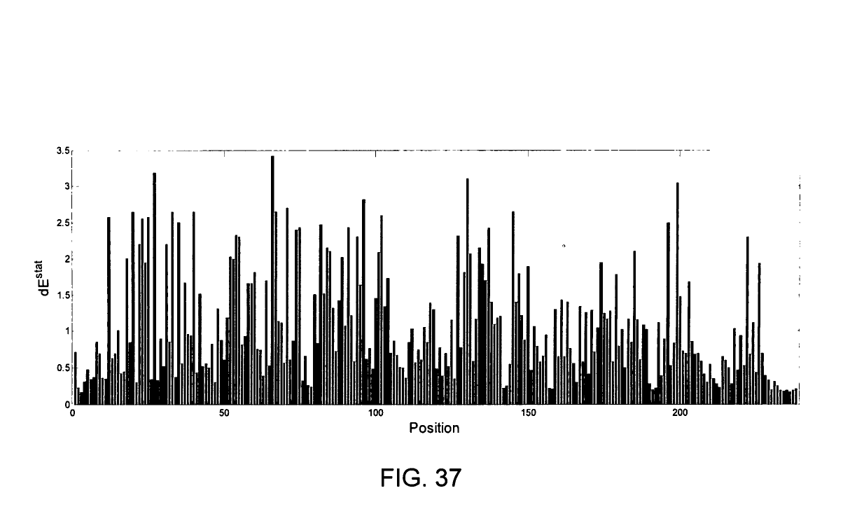

The conservation pattern for this alignment, which was determined (e.g., calculated) using dg.m provided above, is shown in FIG. 37. Several positions show relatively low conservation (dEstat, which is ΔE^{stat}), which indicates that the sequences have had time to evolve away from one another. This suggests that biological position elements that are similar between proteins have been naturally selected to be this way.

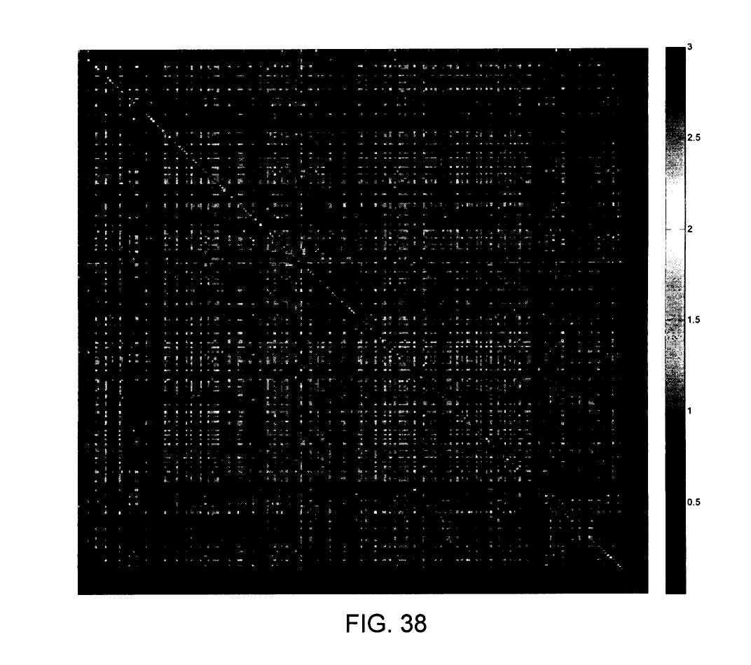

A cmr matrix like the one created for the WW and PDZ domains was created for the alignment of fluorescent proteins, as shown in FIG. 38. As with the WW and PDZ domains, the cmr matrix for the fluorescent proteins shows sparse evolutionarily-coupled positions in the alignment, with a subset of positions that show similar patterns of strong coupling.

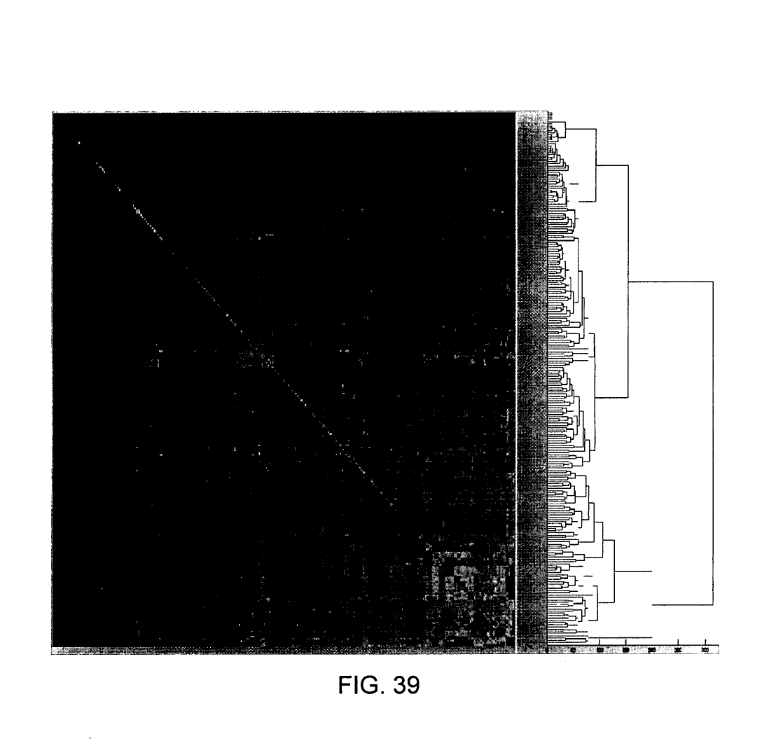

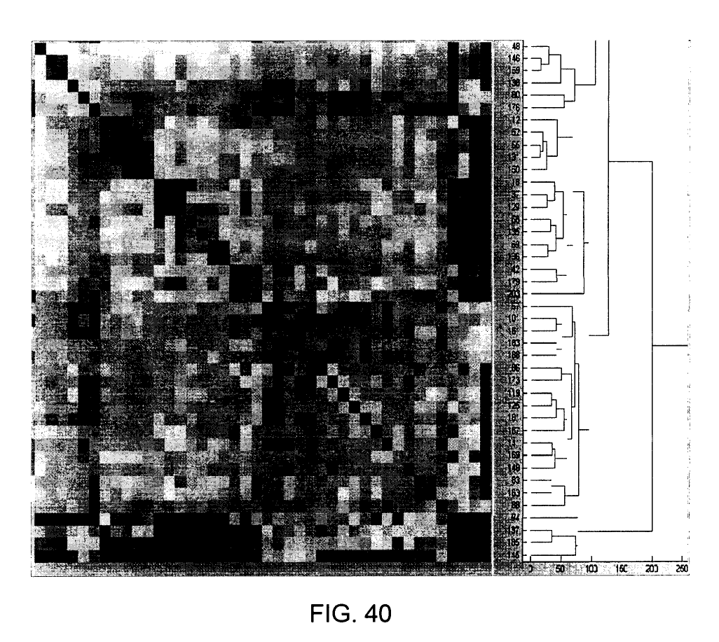

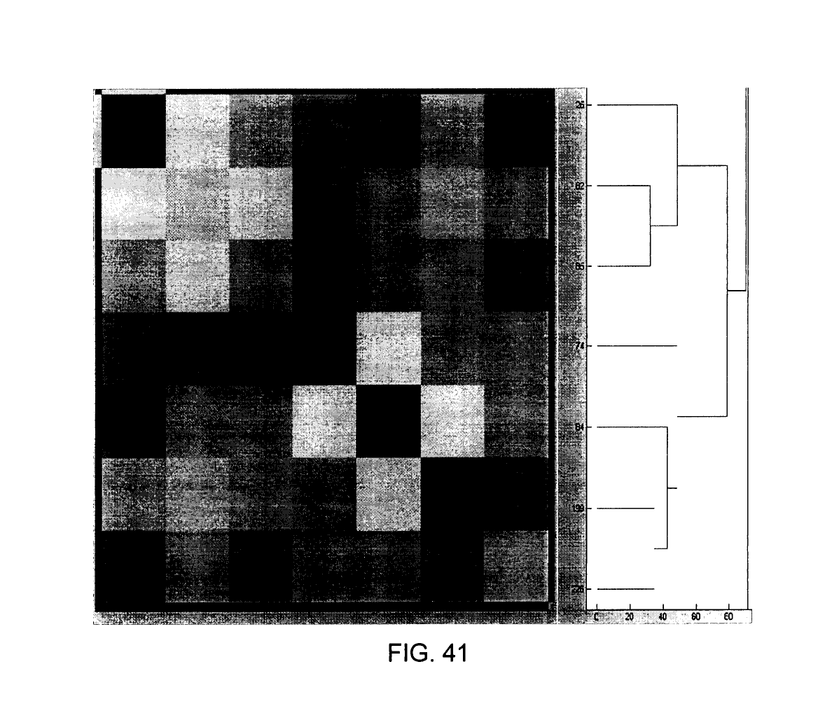

The clustering technique described above in section 4.1 was applied to the cmr matrix shown in FIG. 38 to produce the matrix shown in FIG. 39, which comprises a more detailed version of a SCA. FIG. 40 is an enlarged detail view of a portion of the matrix shown in FIG. 39, and reveals that positions 12, 18, 37, 42, 48, 52, 55, 57, 58, 59, 80, 83, 86, 88, 94, 101, 119, 125, 129, 131, 135, 136, 137, 138, 141, 145, 146, 148, 150, 159, 161, 163, 167, 169, 173, 176, 179, 181, 183, 185, 188, and 203 comprise a co-evolving network. FIG. 41 is a more enlarged detail view of a smaller portion of the matrix shown in FIG. 39, and reveals that a separate network of positions 25, 74, 82, 84, 85, 199 and 226 are co-evolving with each other, but not with the larger cluster.

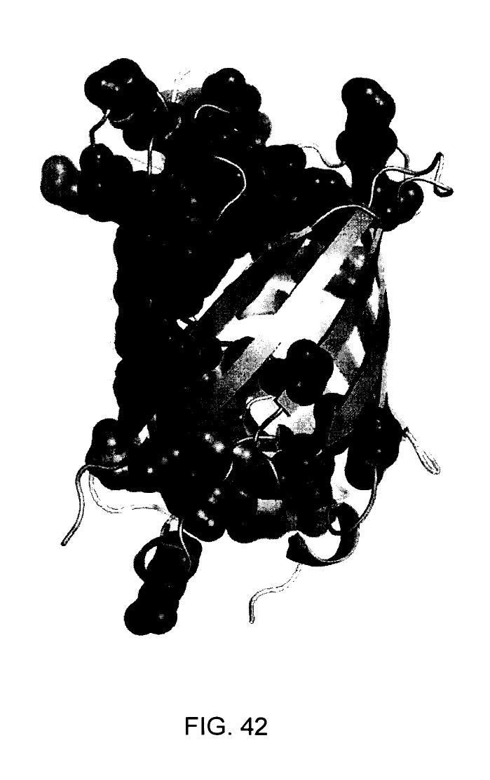

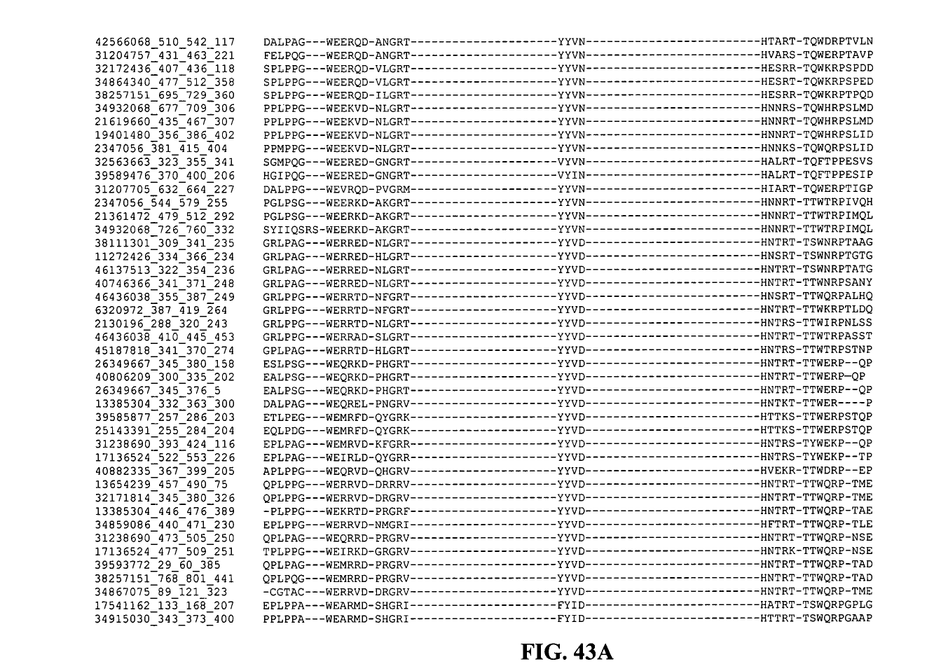

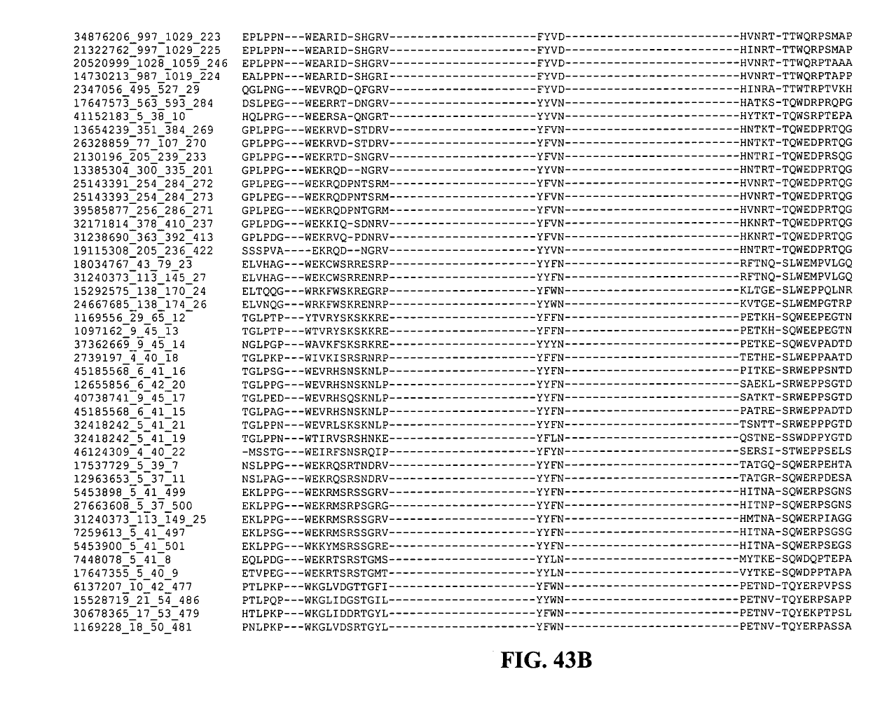

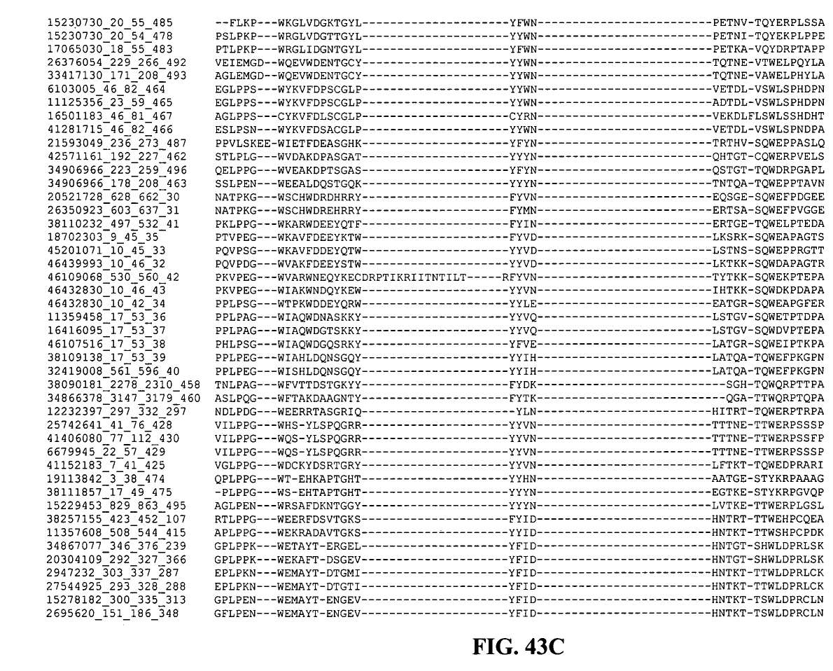

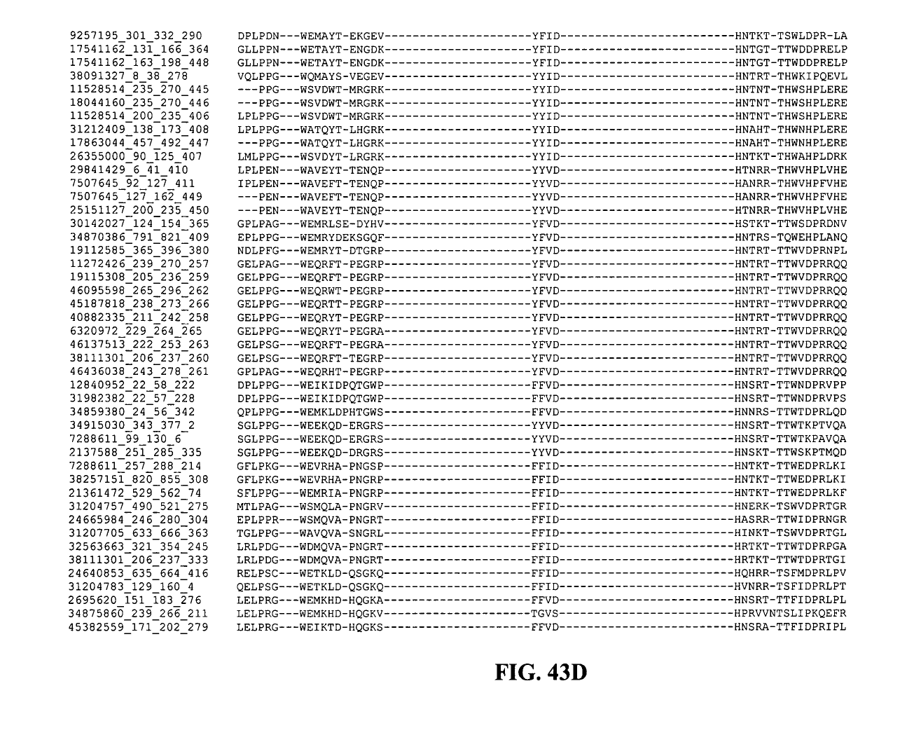

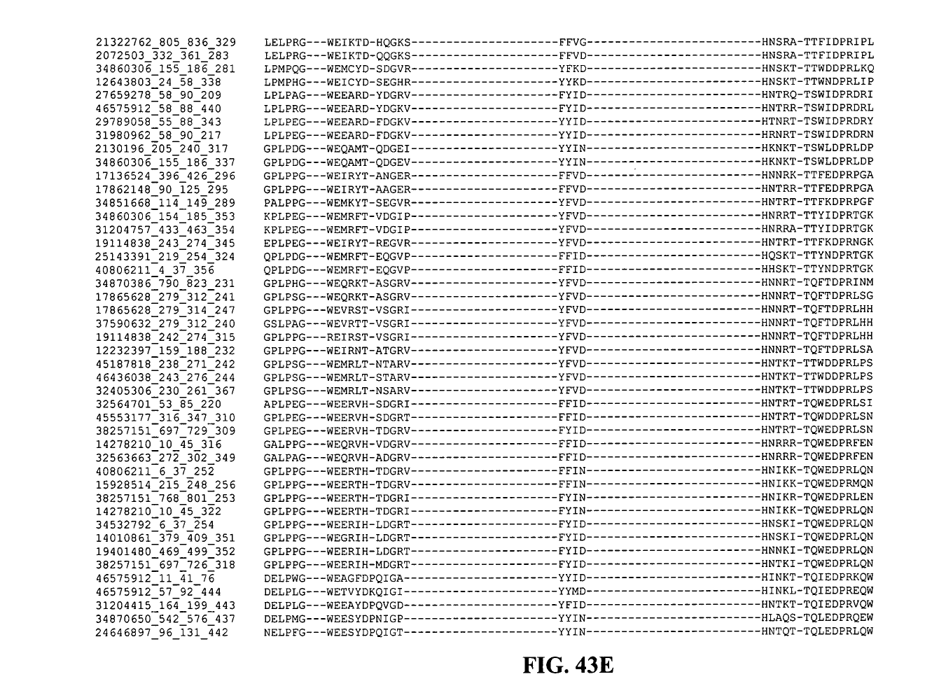

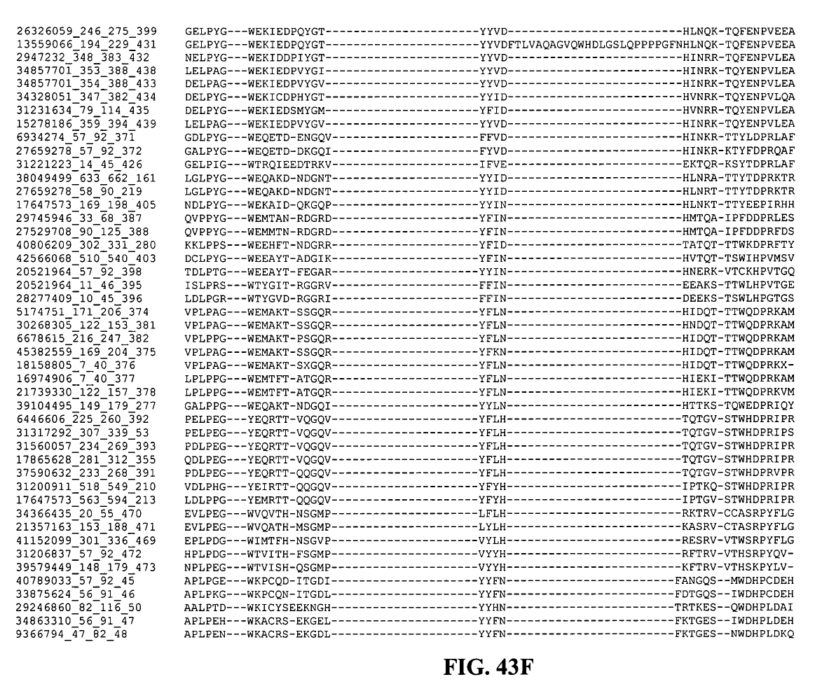

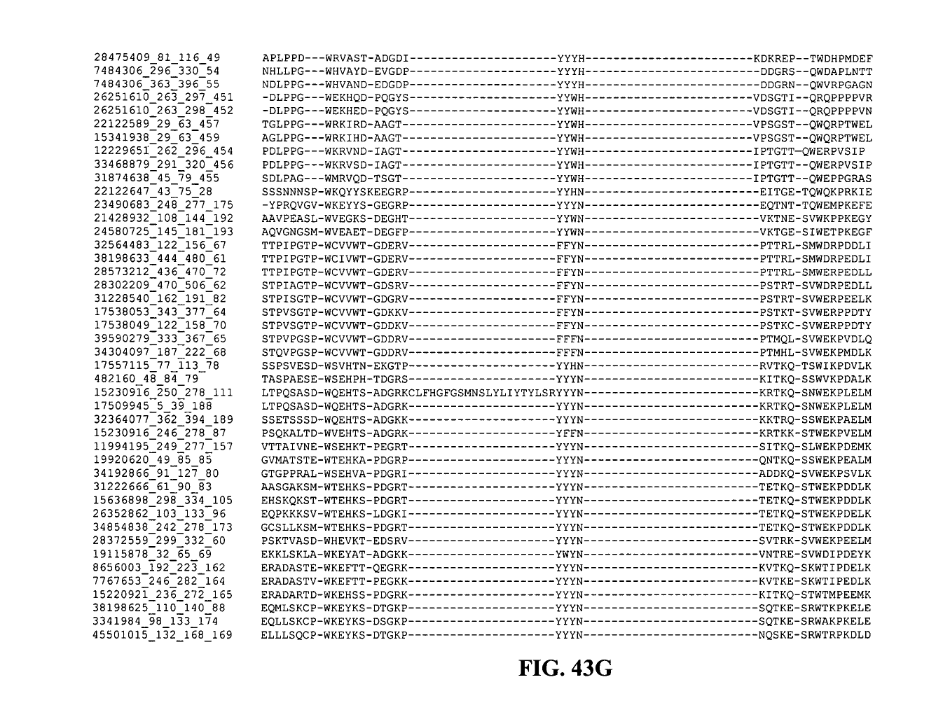

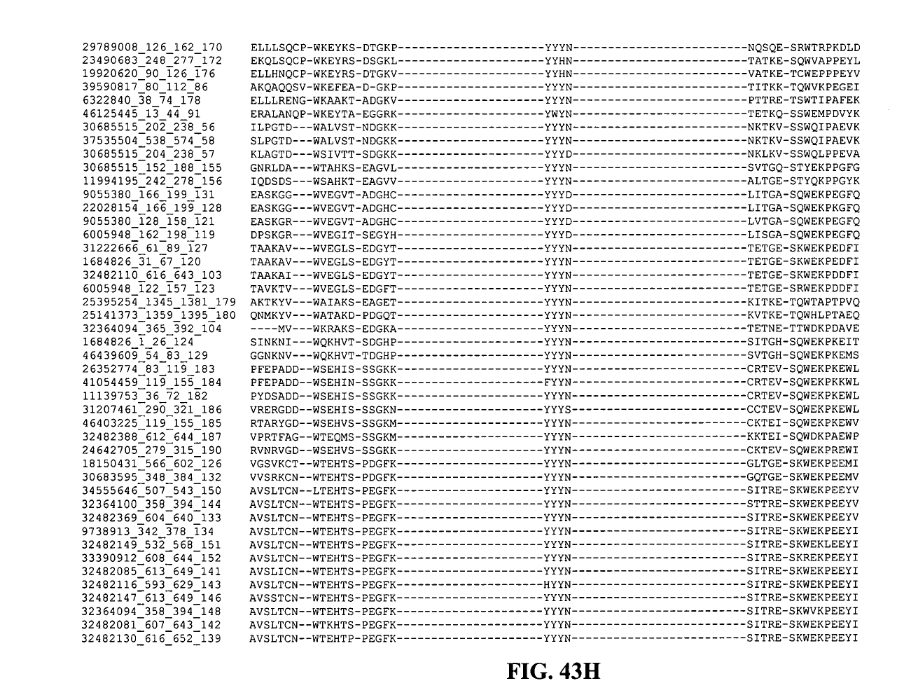

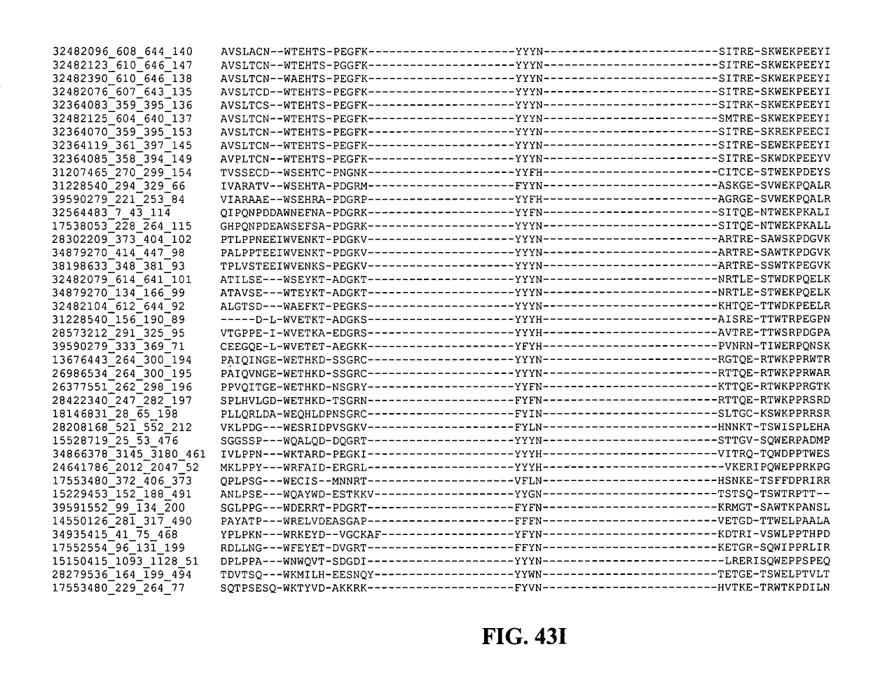

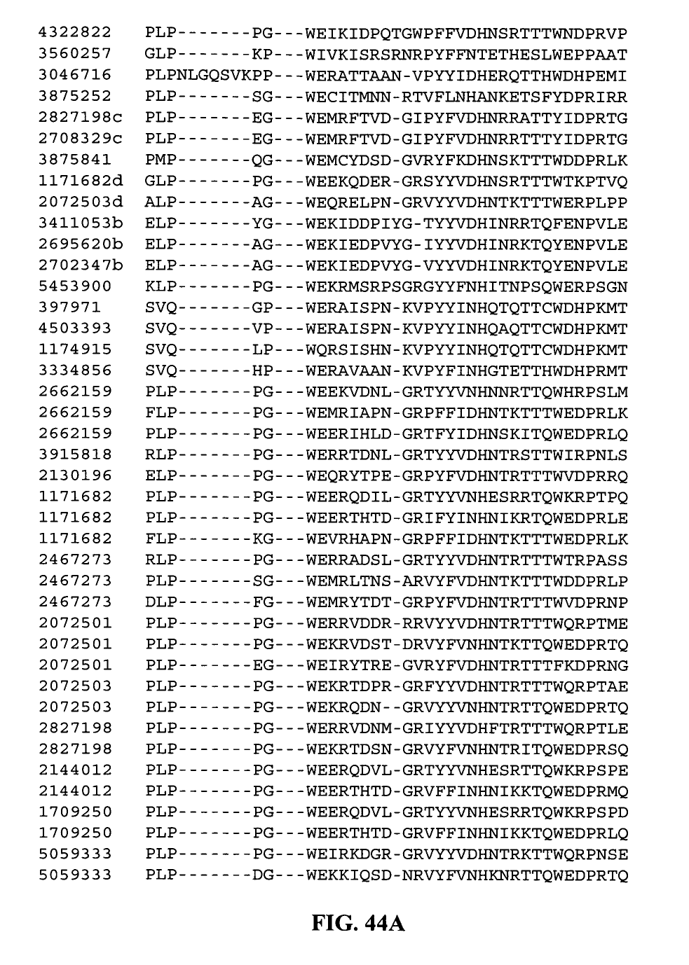

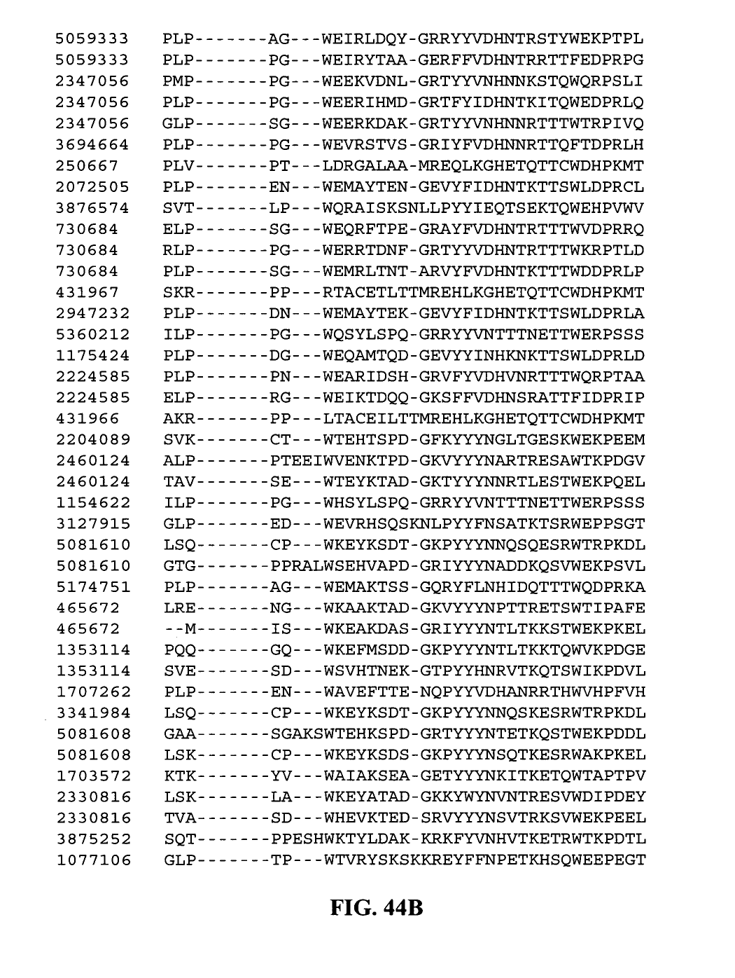

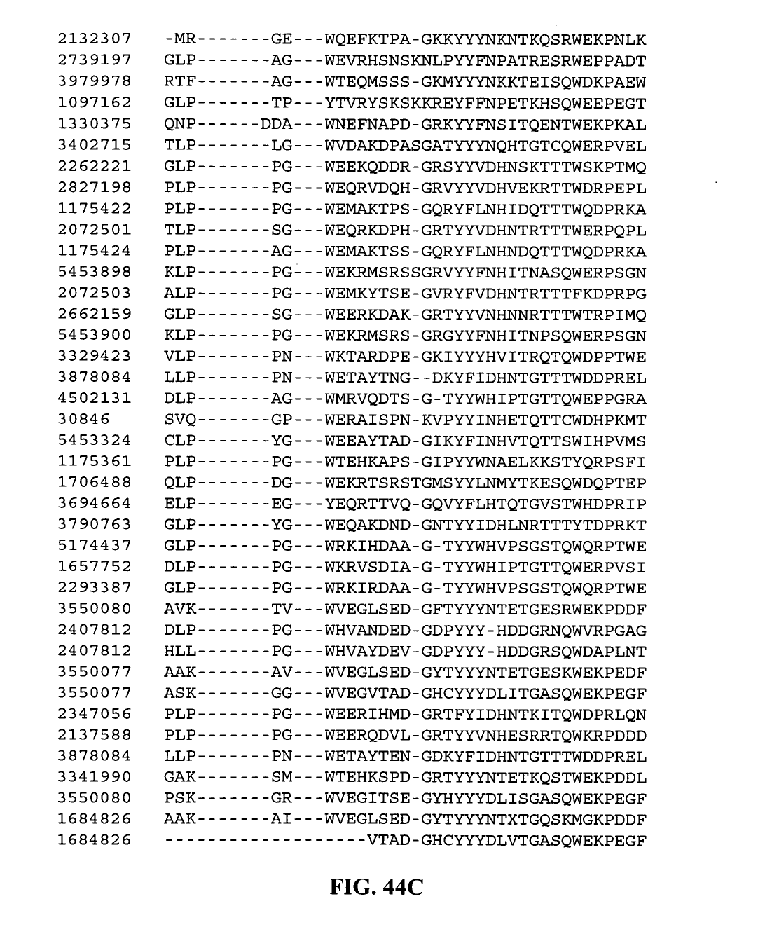

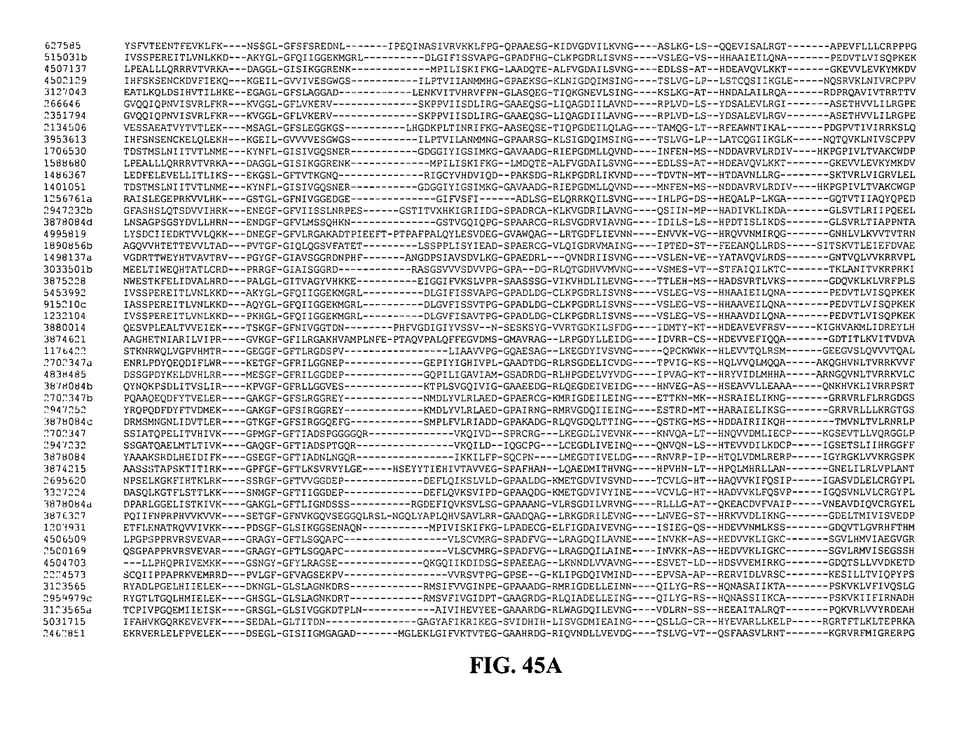

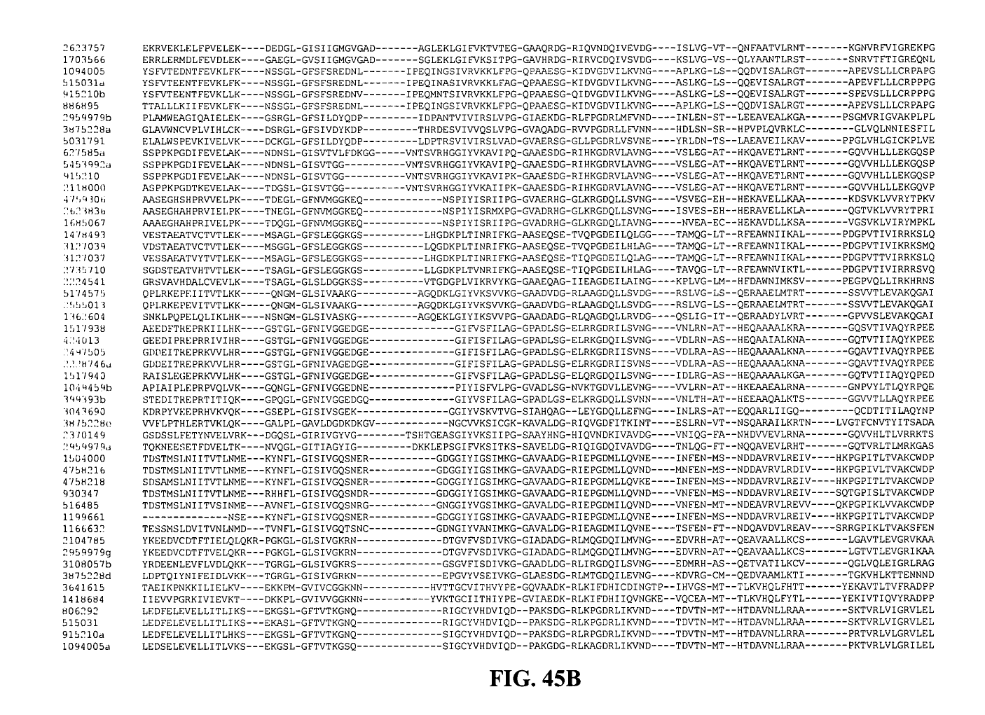

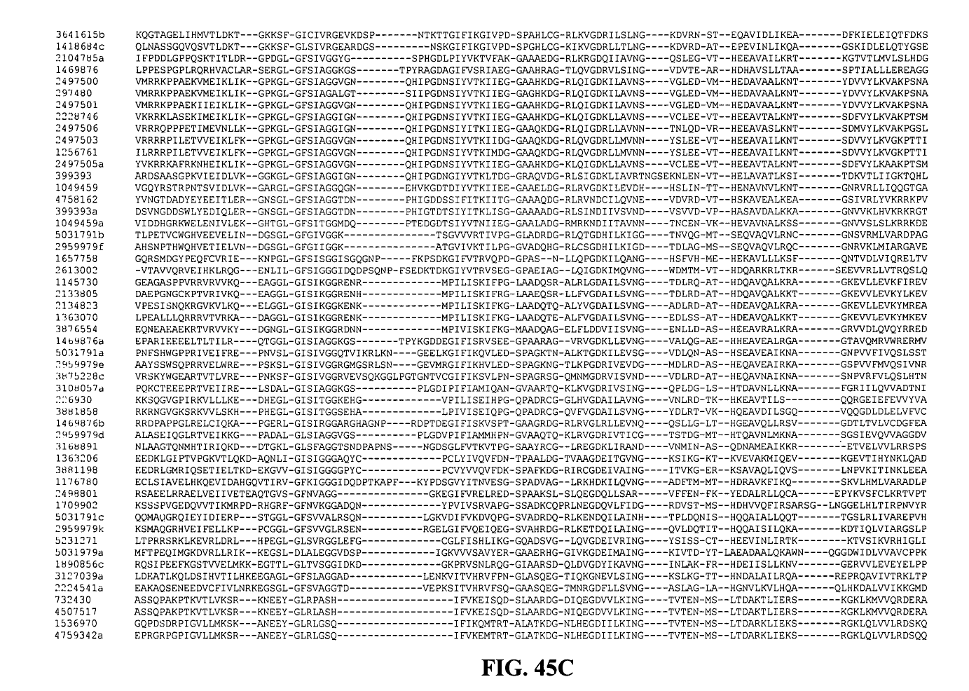

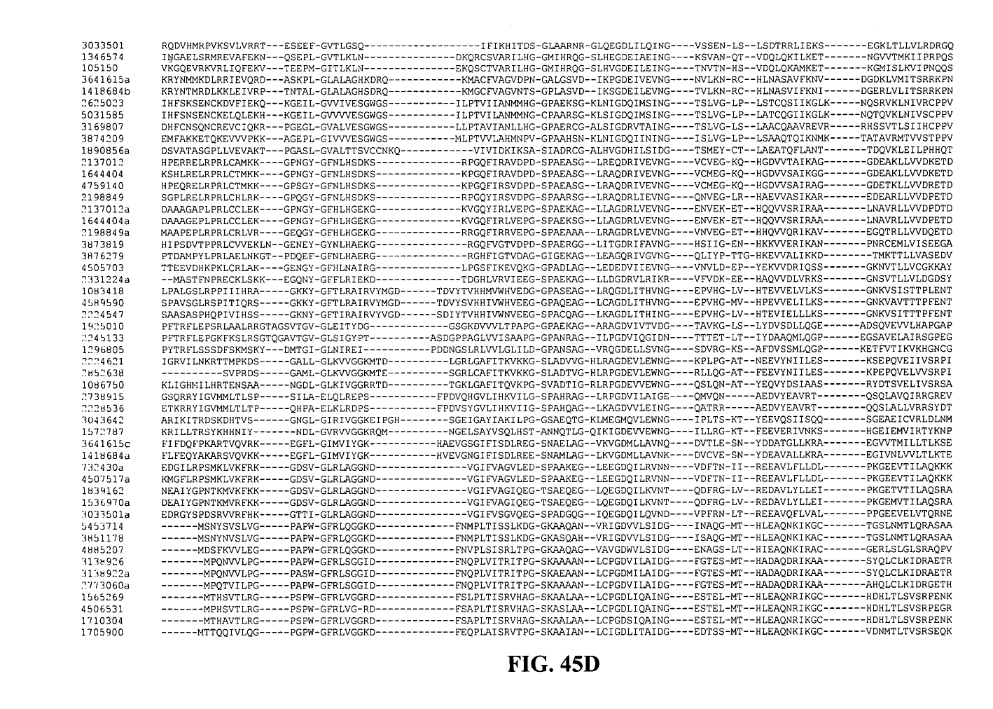

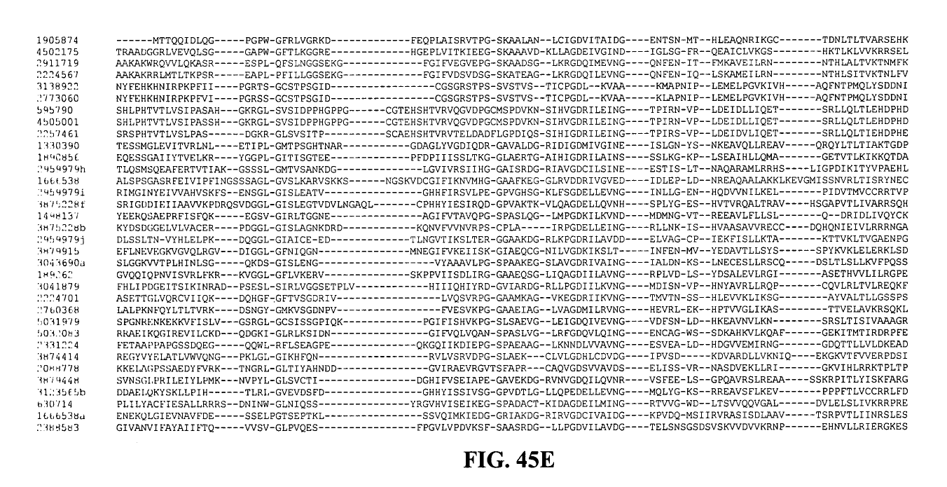

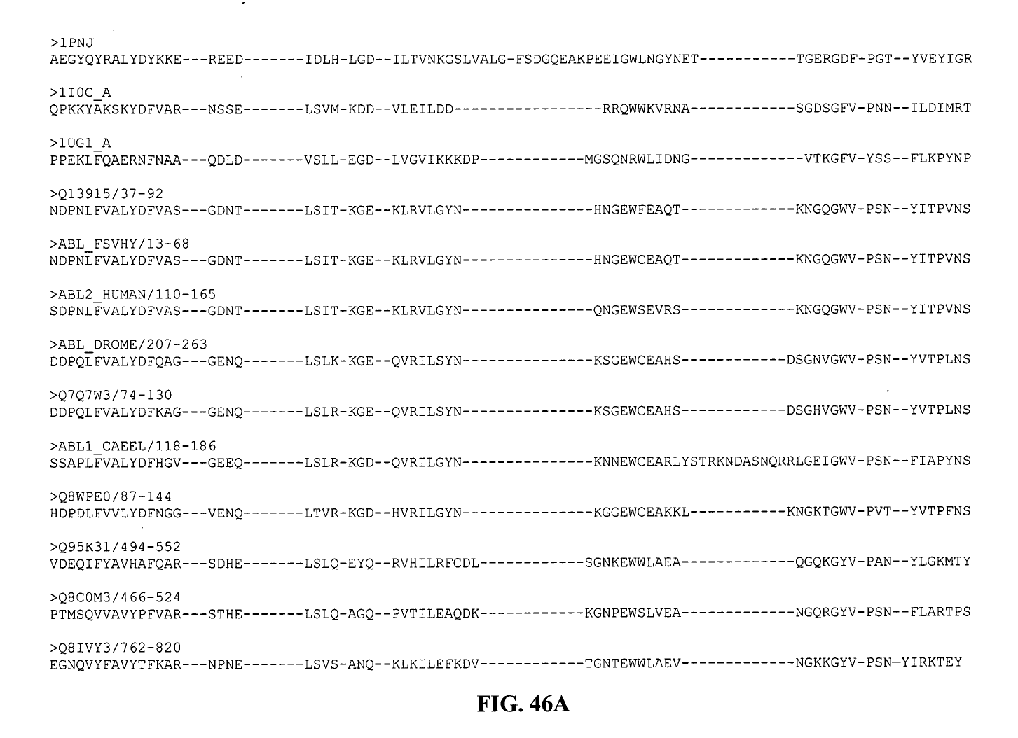

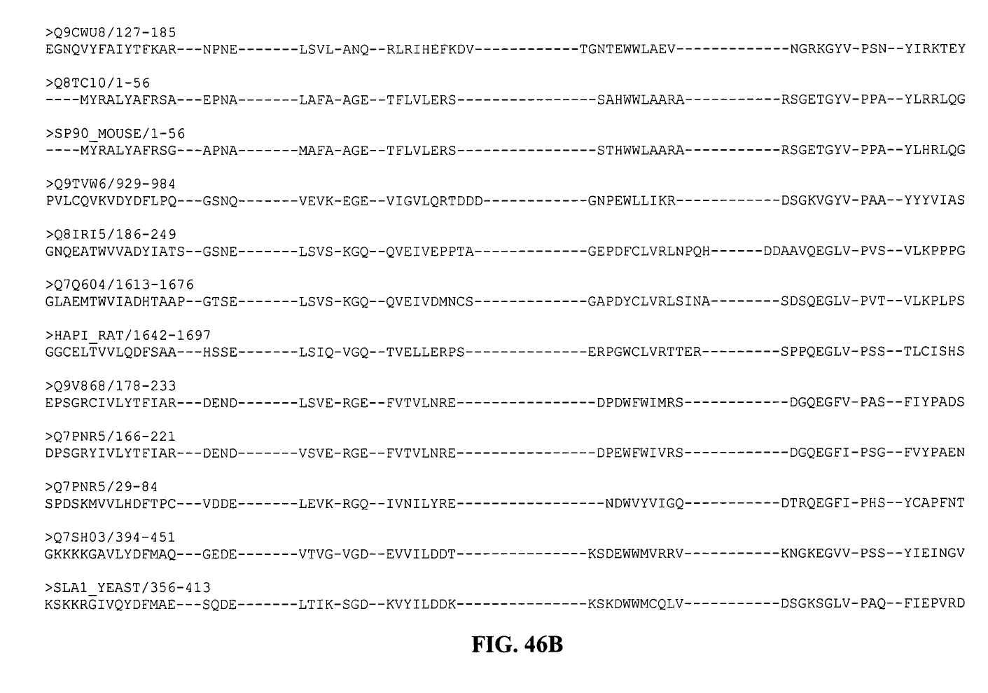

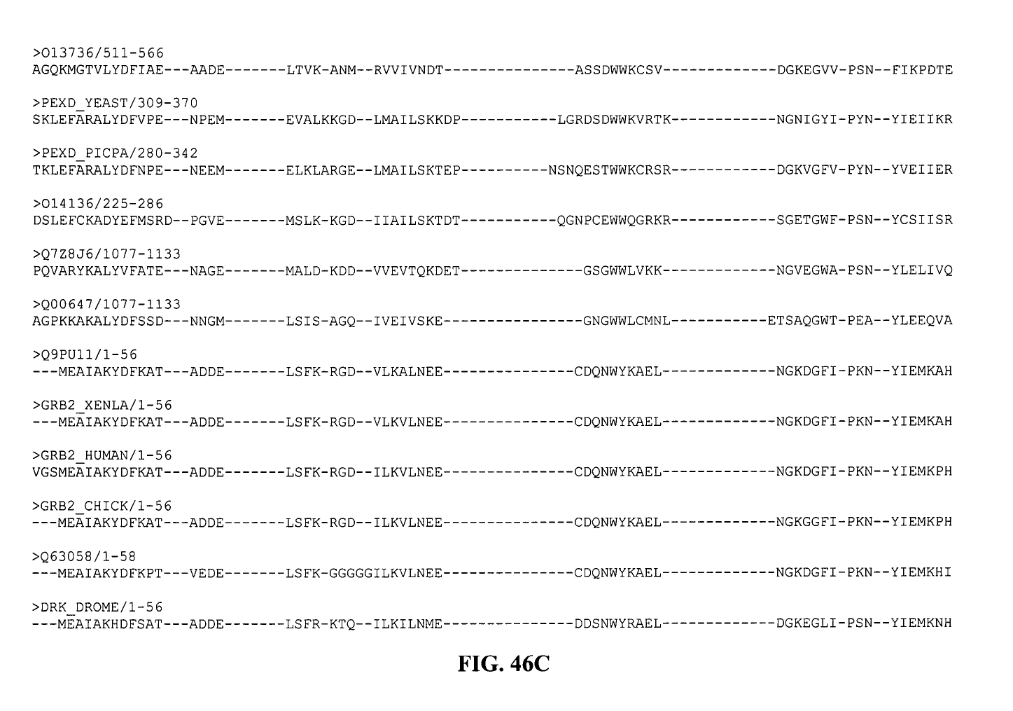

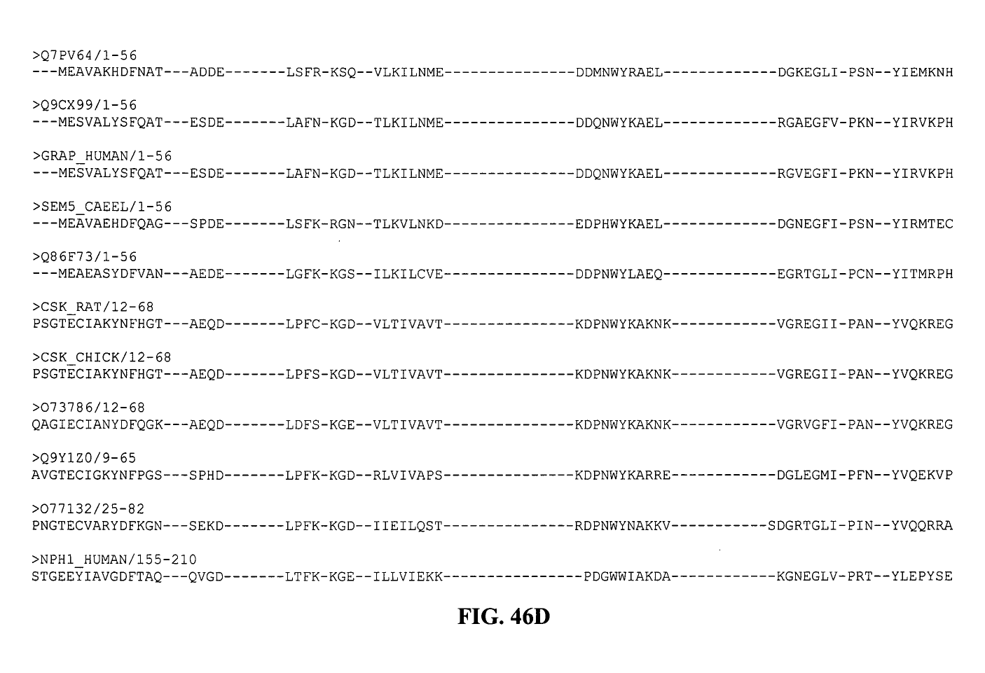

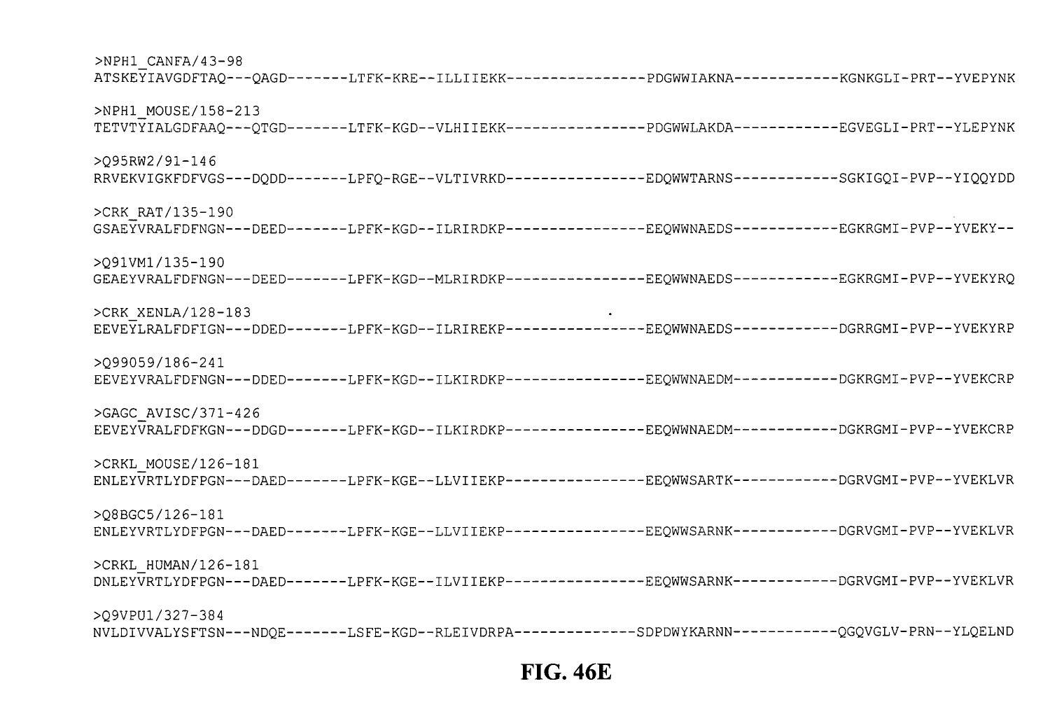

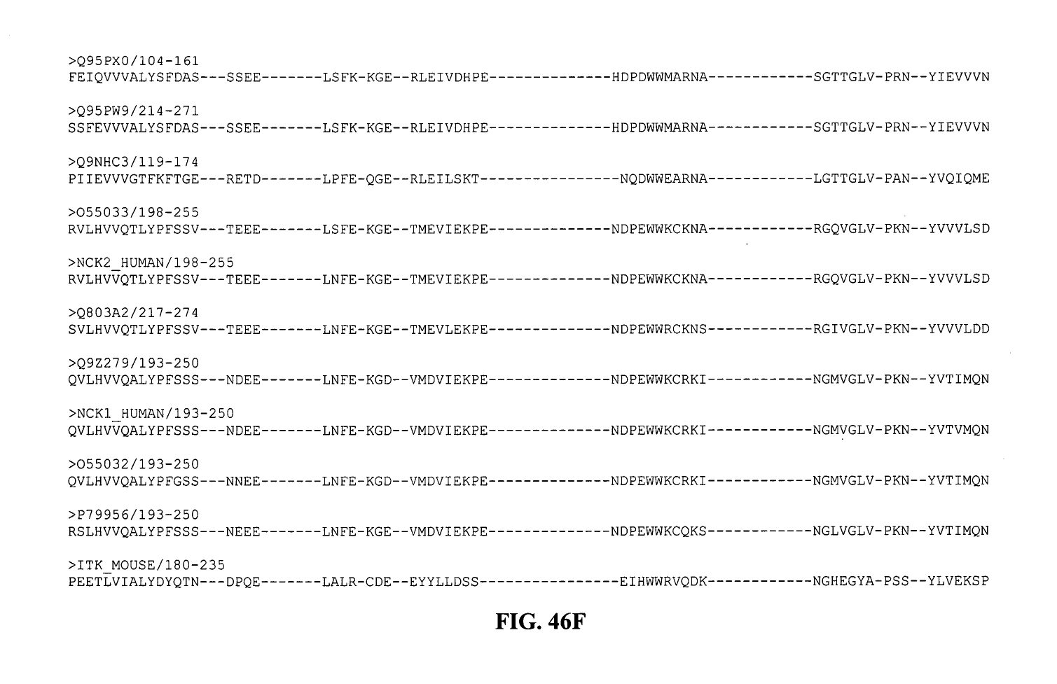

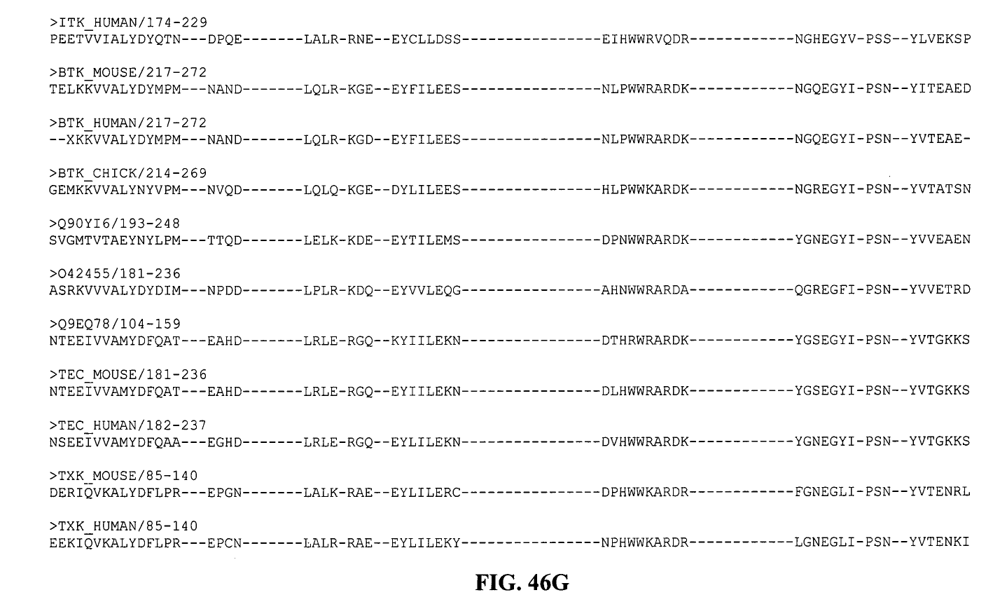

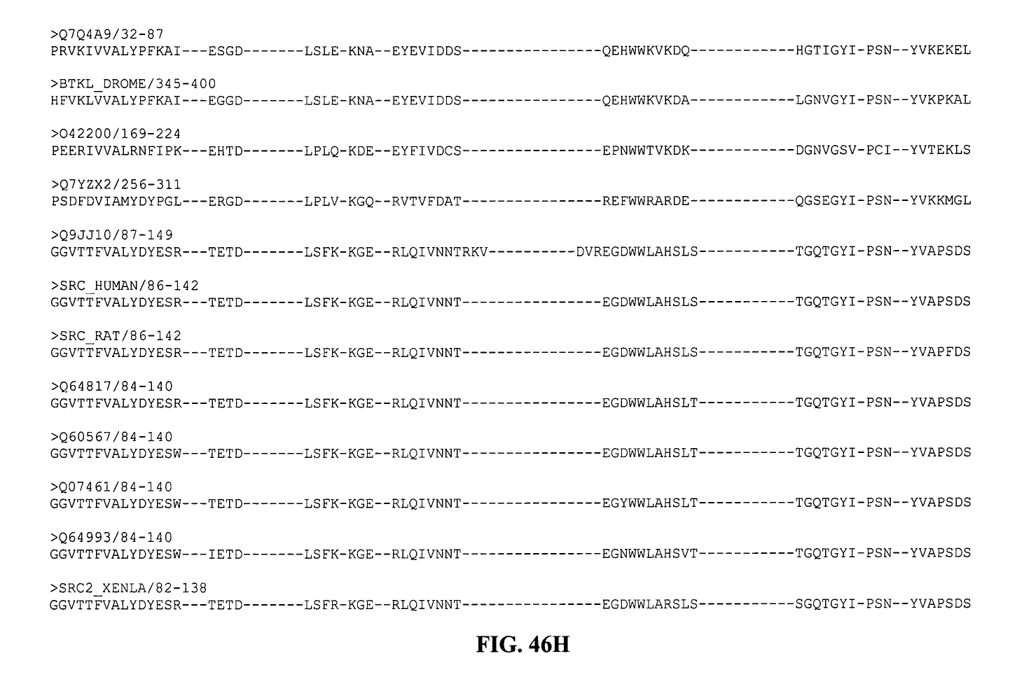

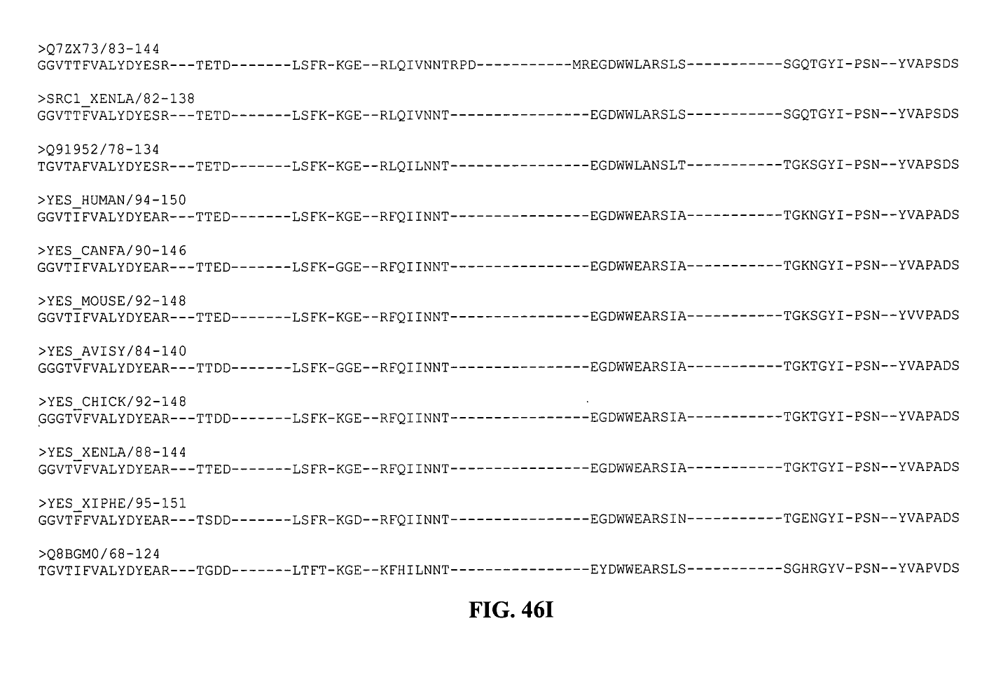

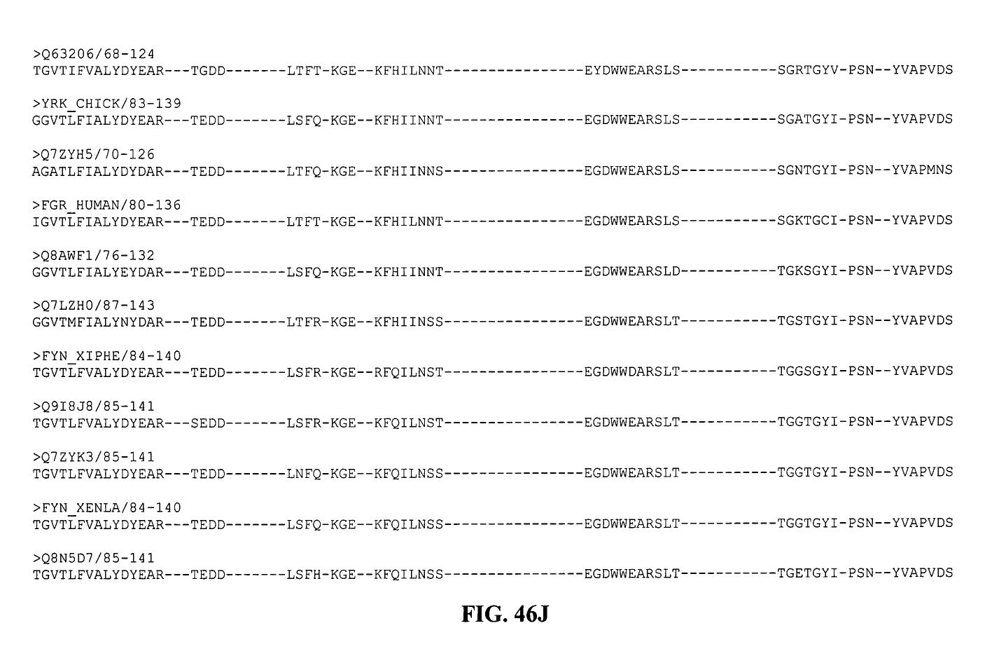

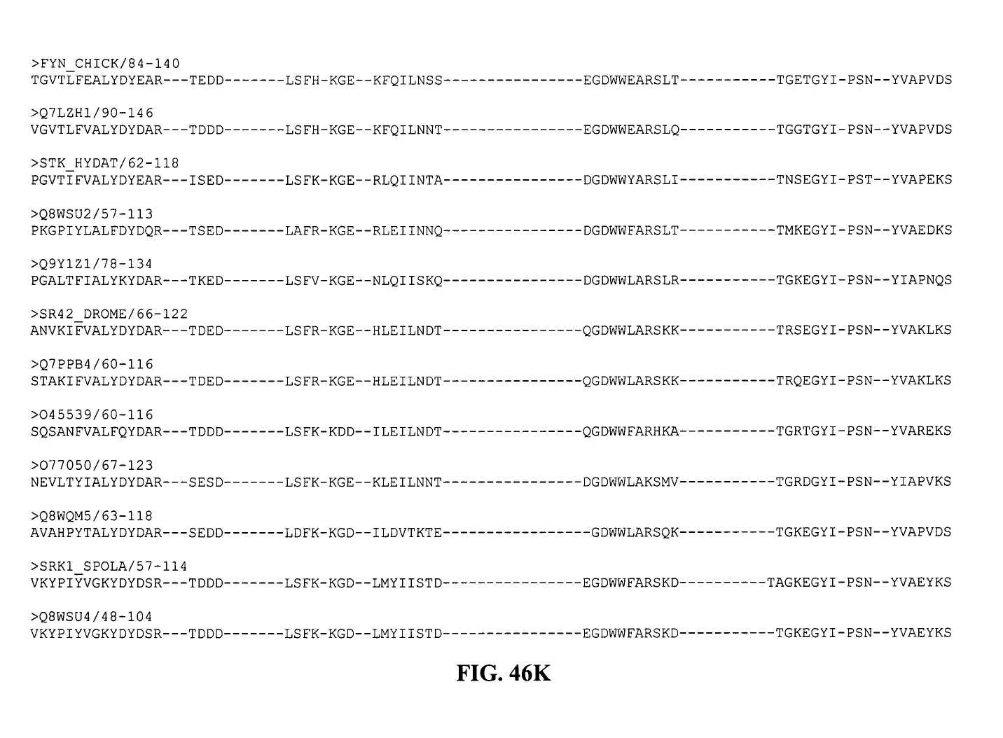

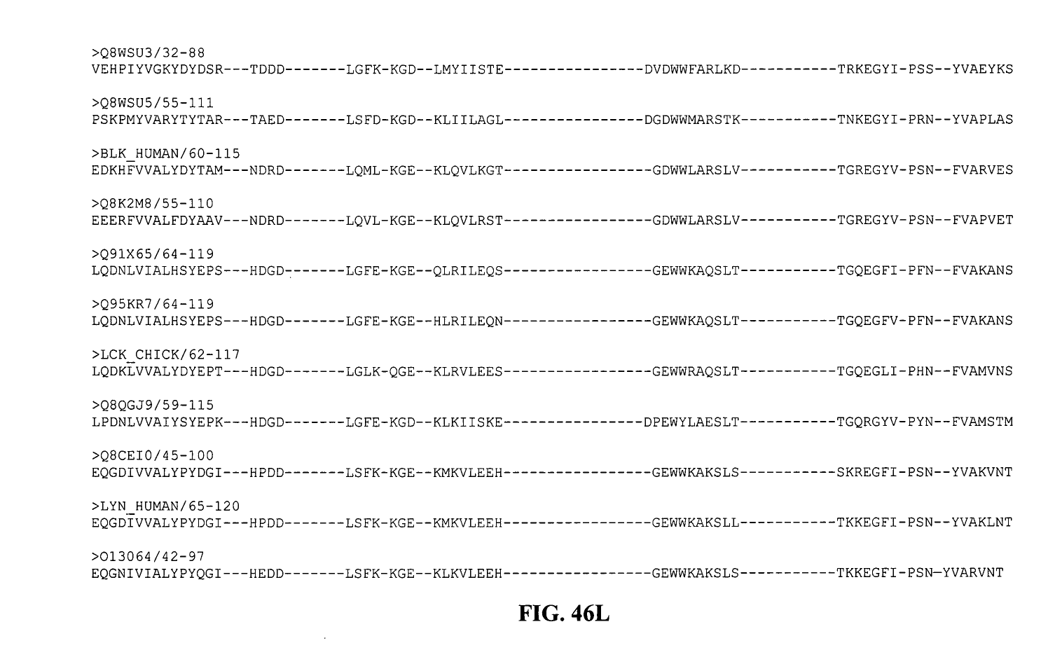

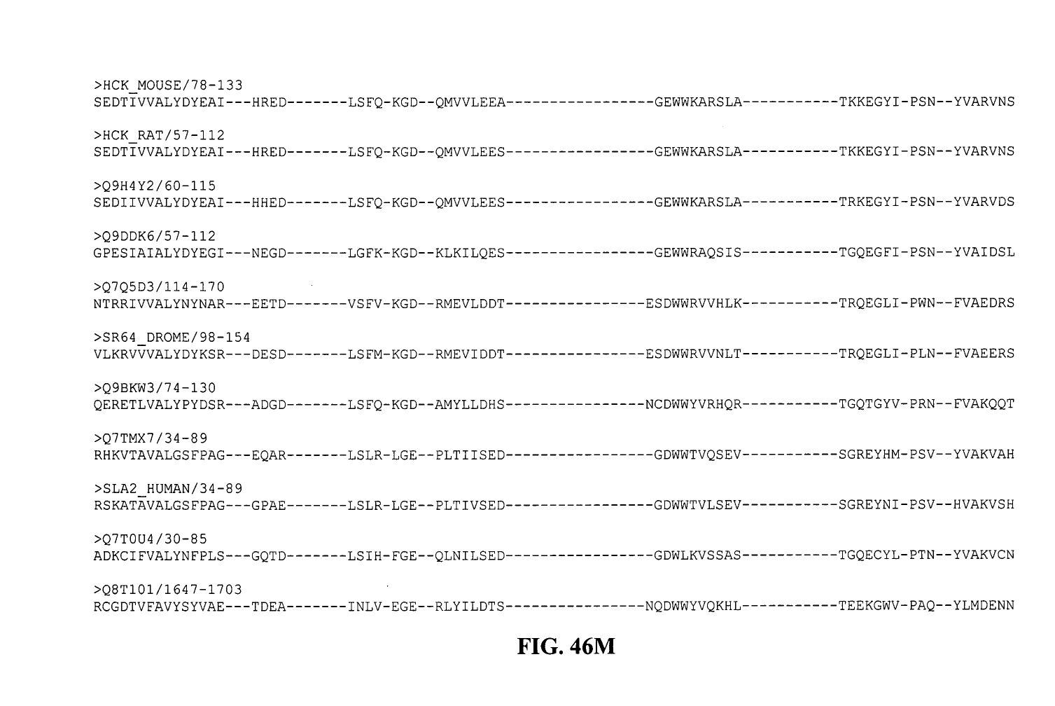

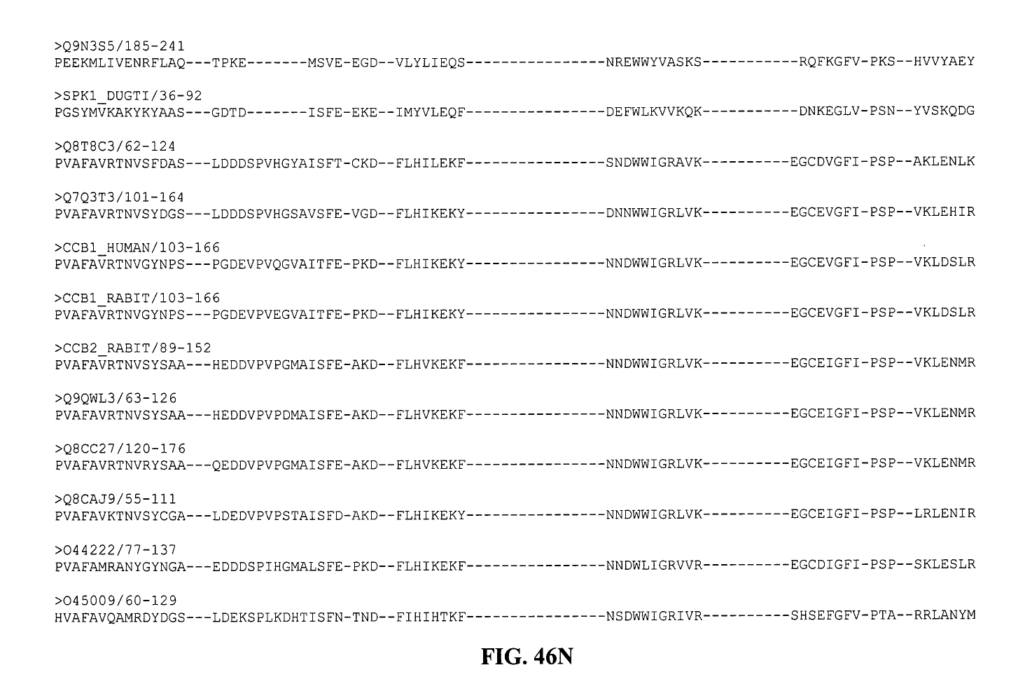

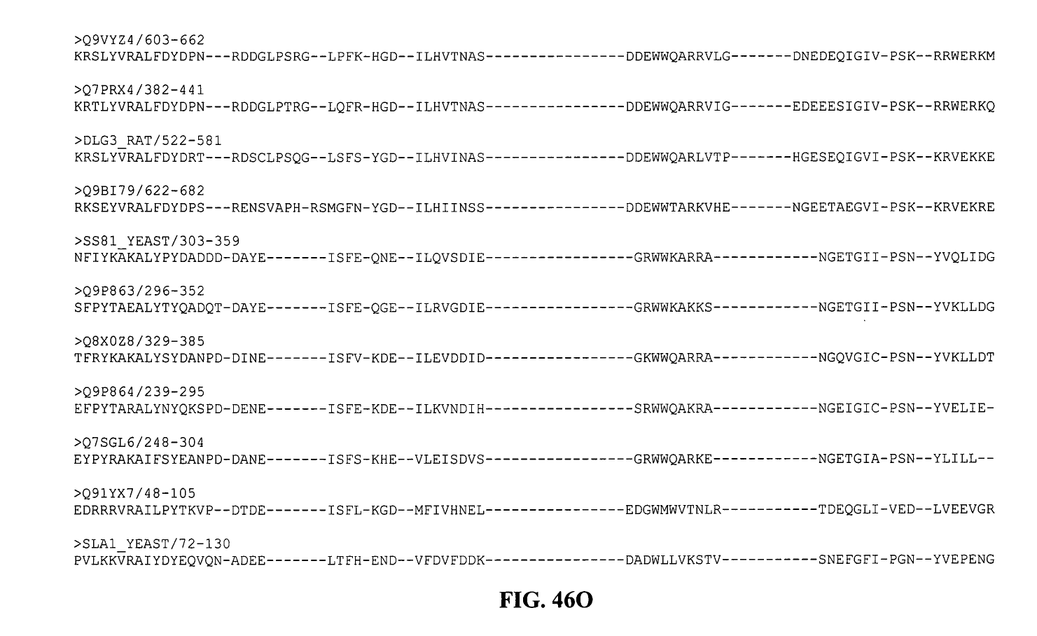

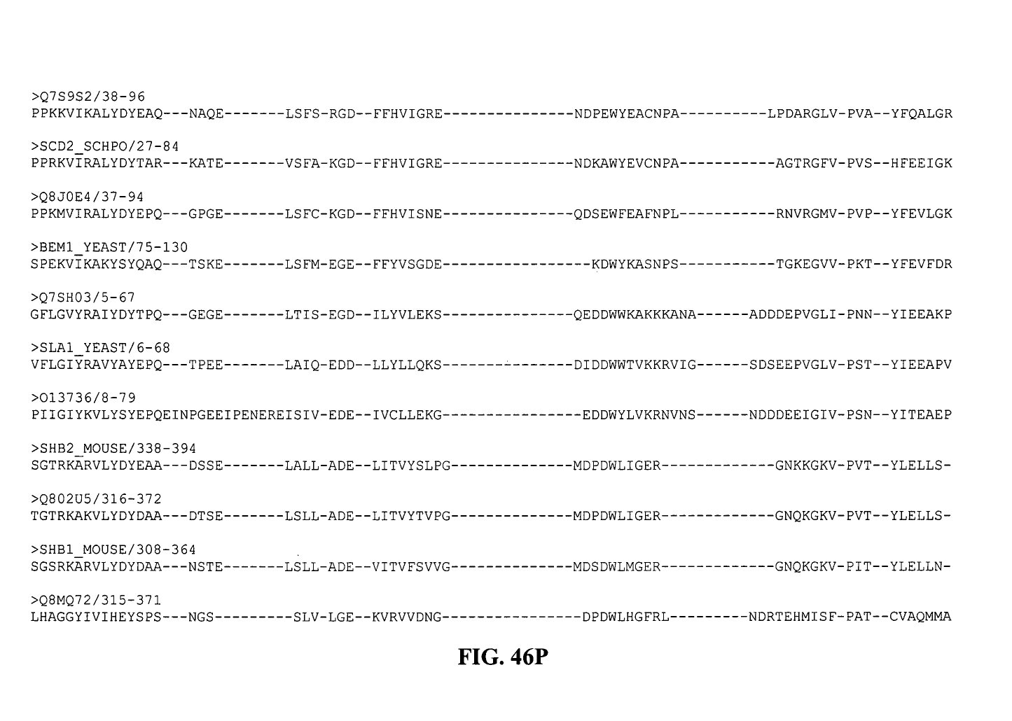

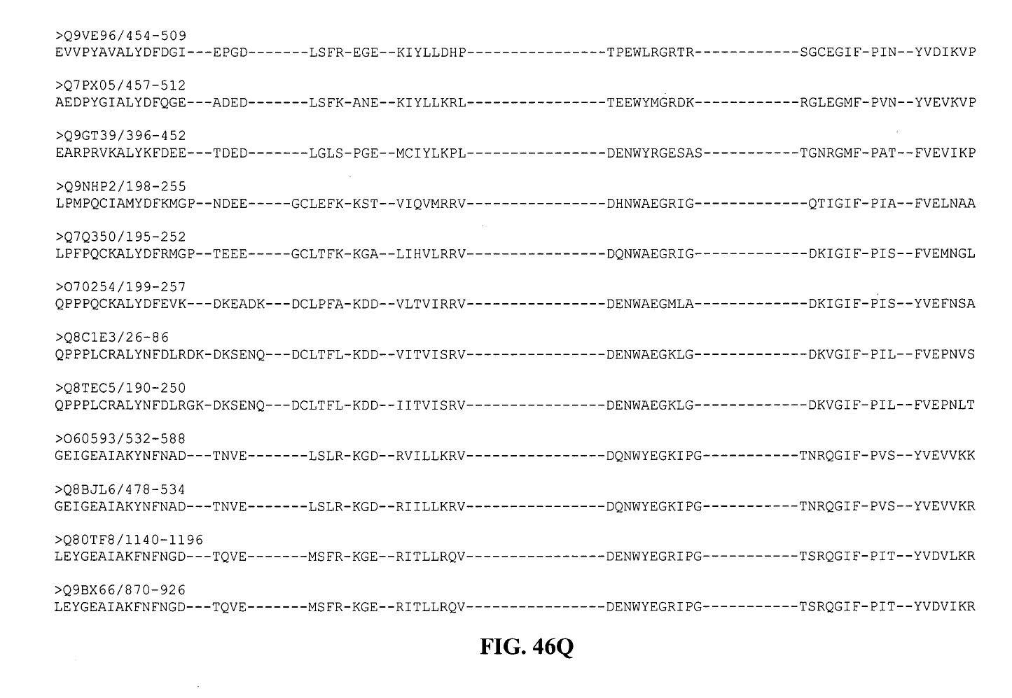

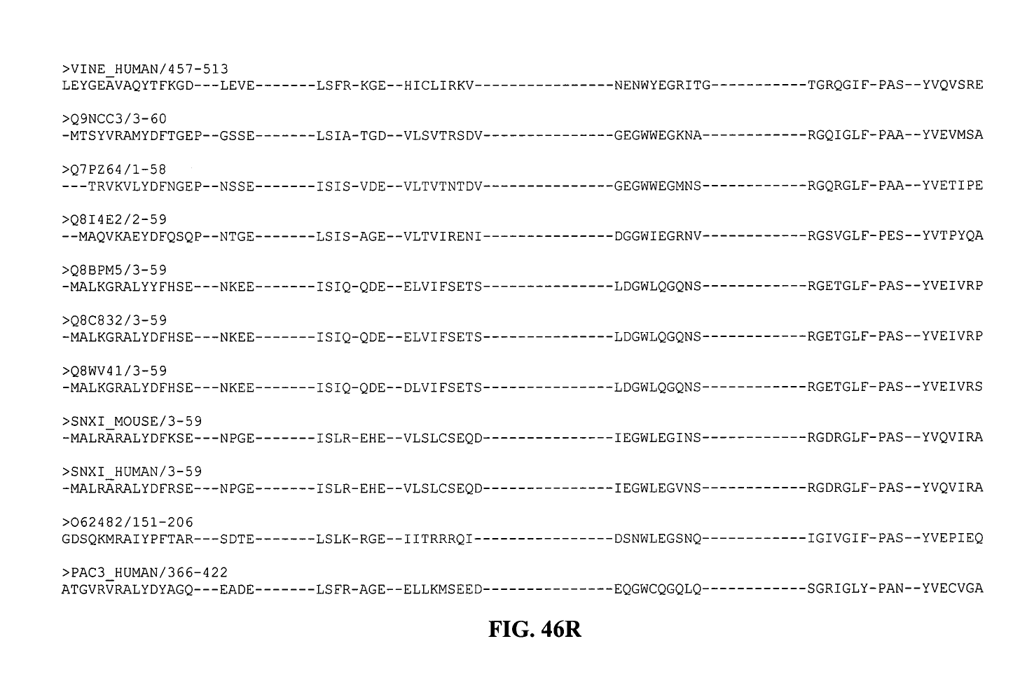

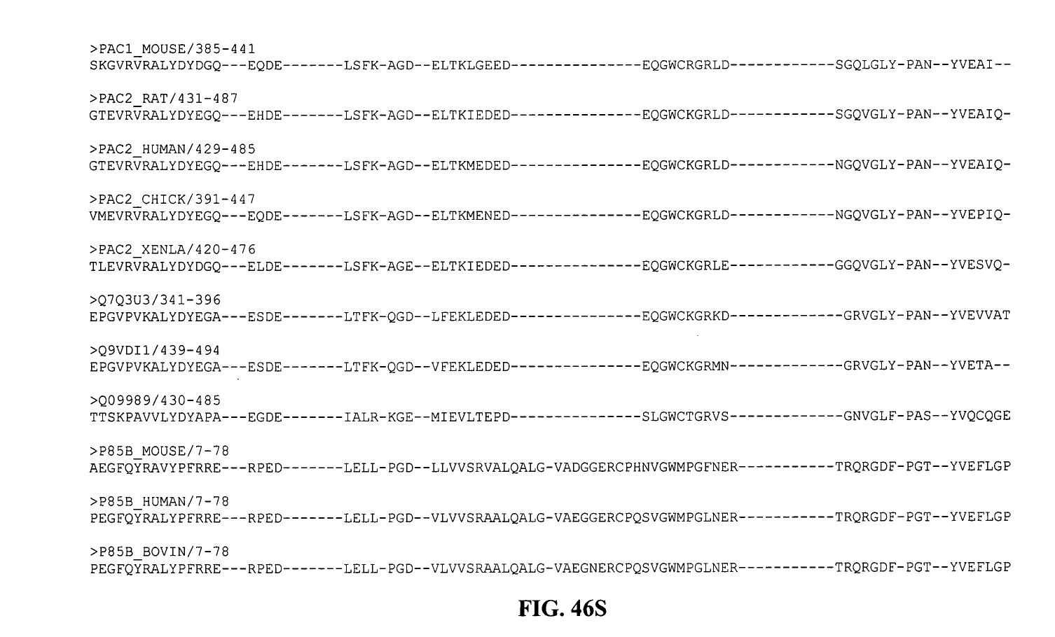

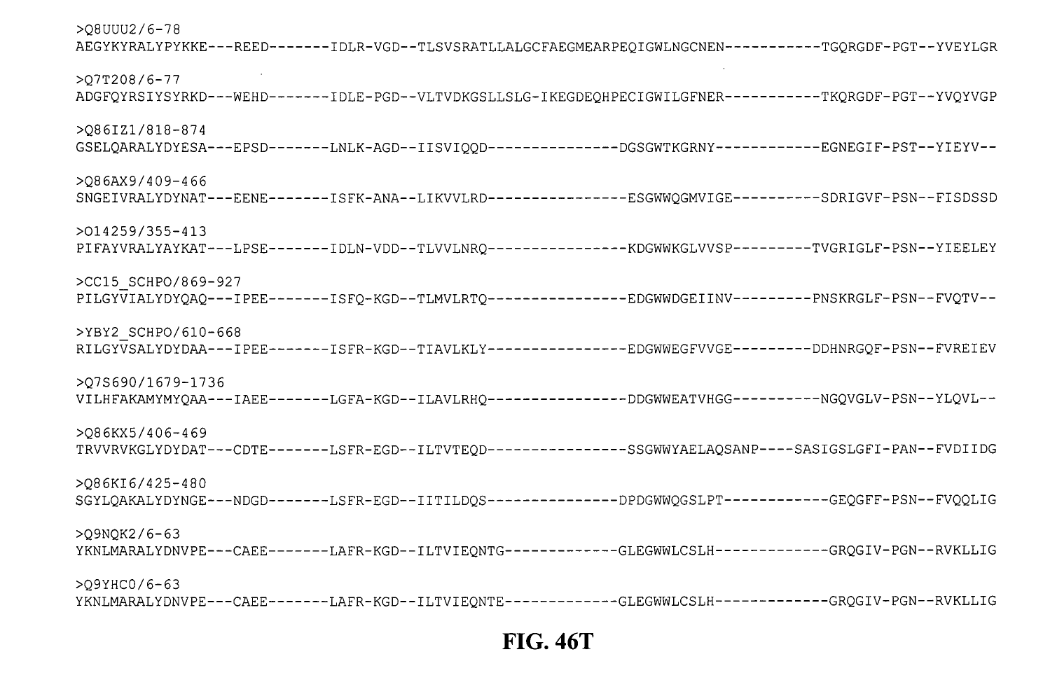

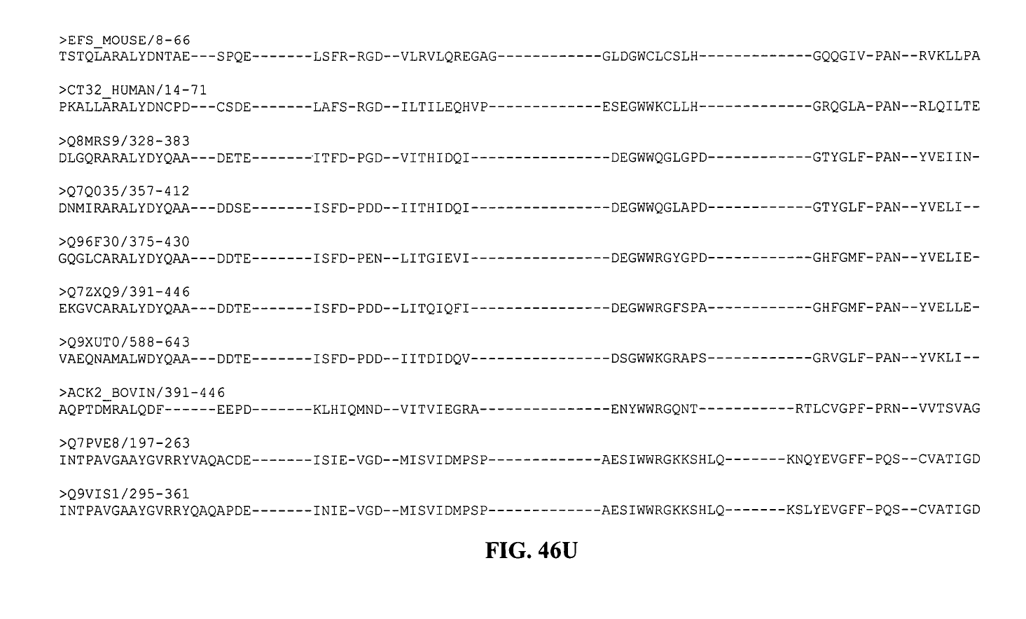

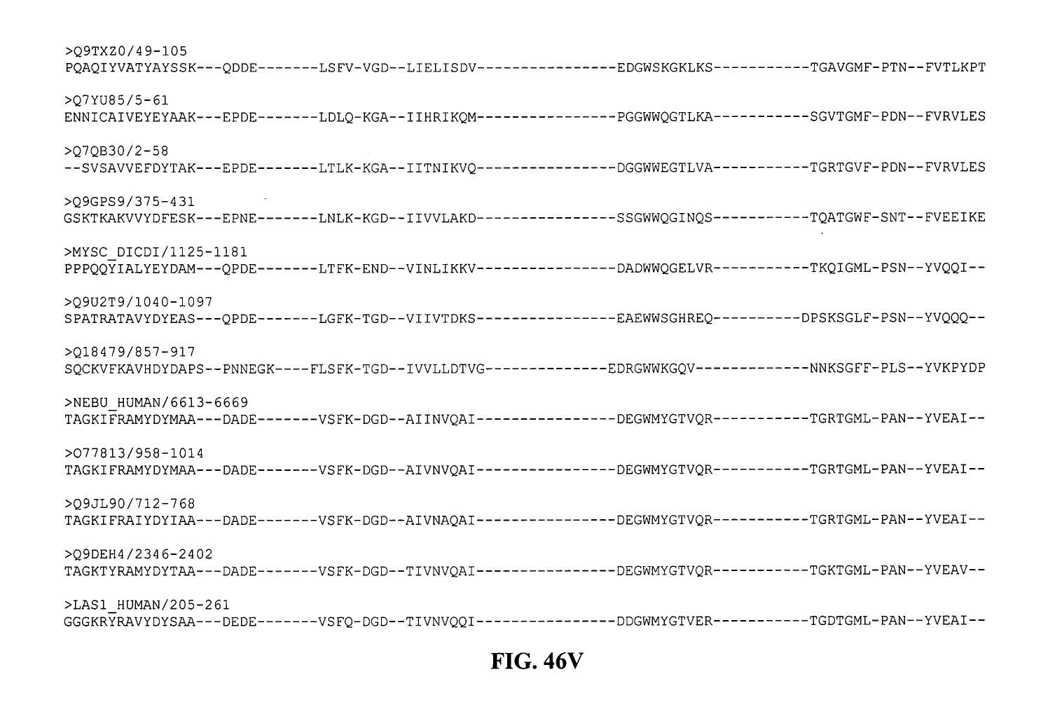

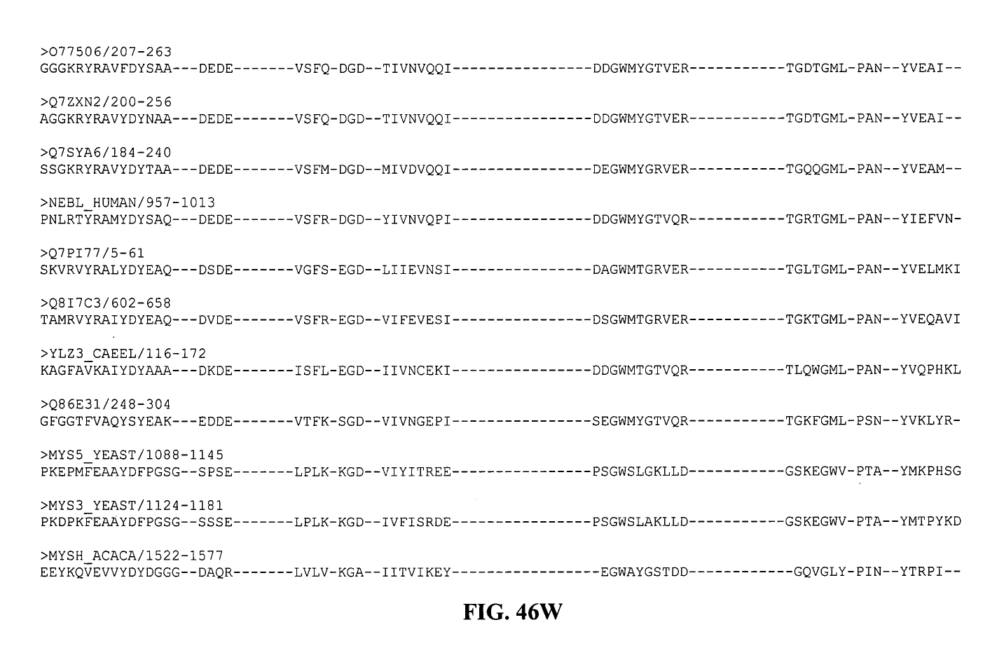

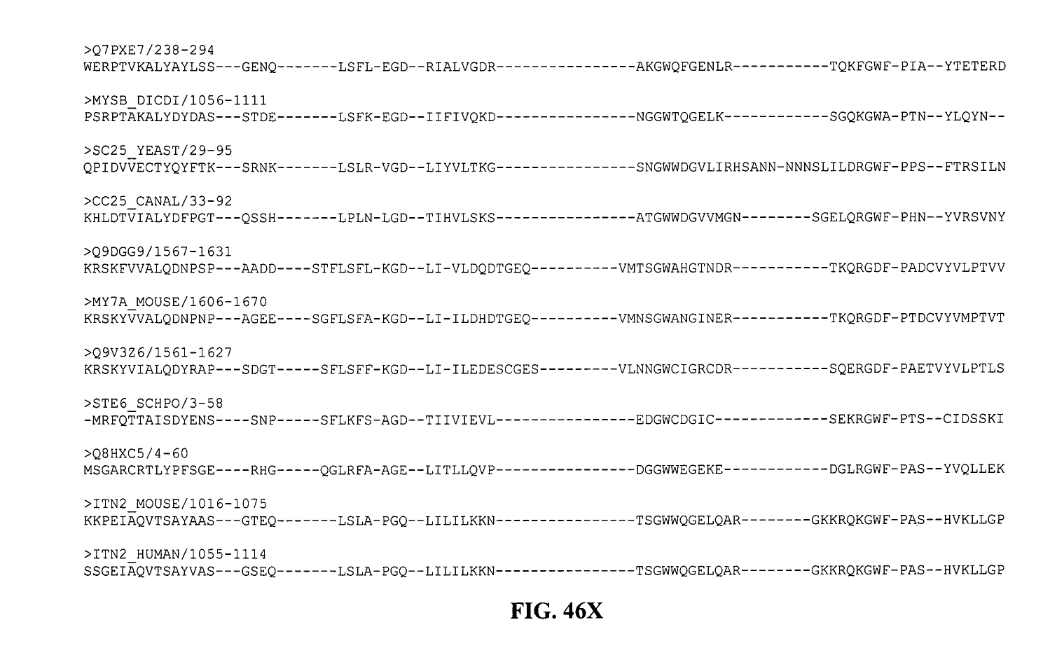

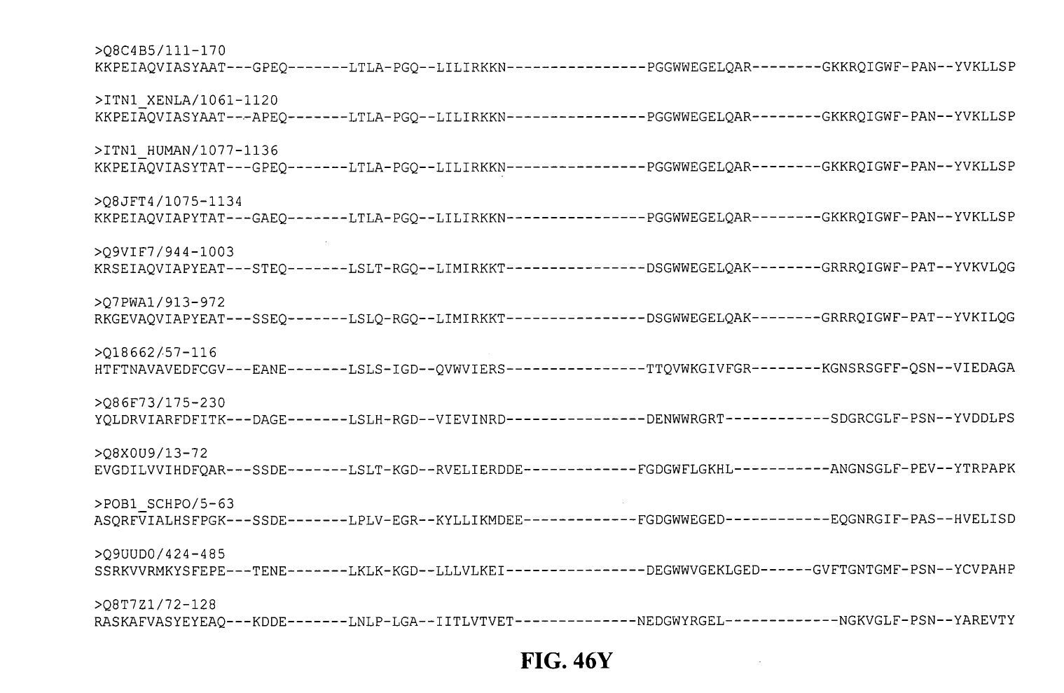

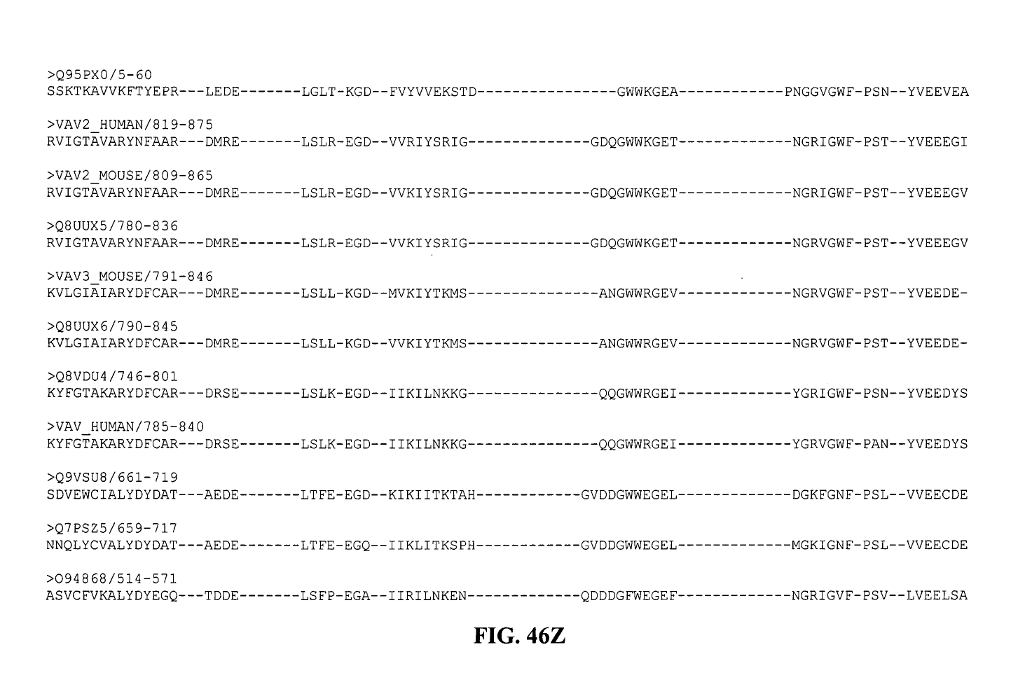

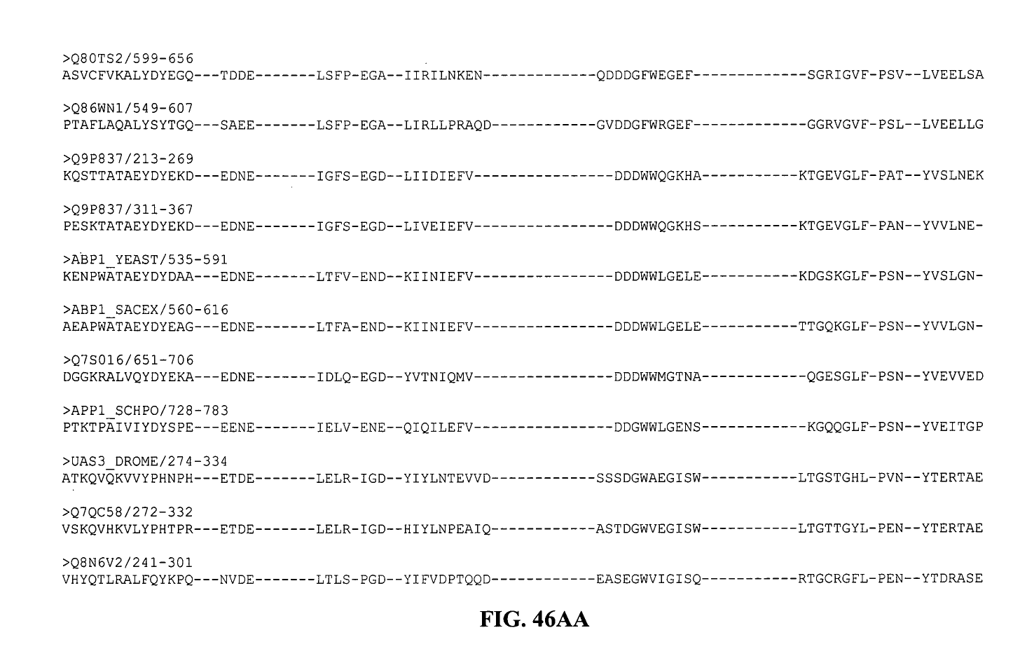

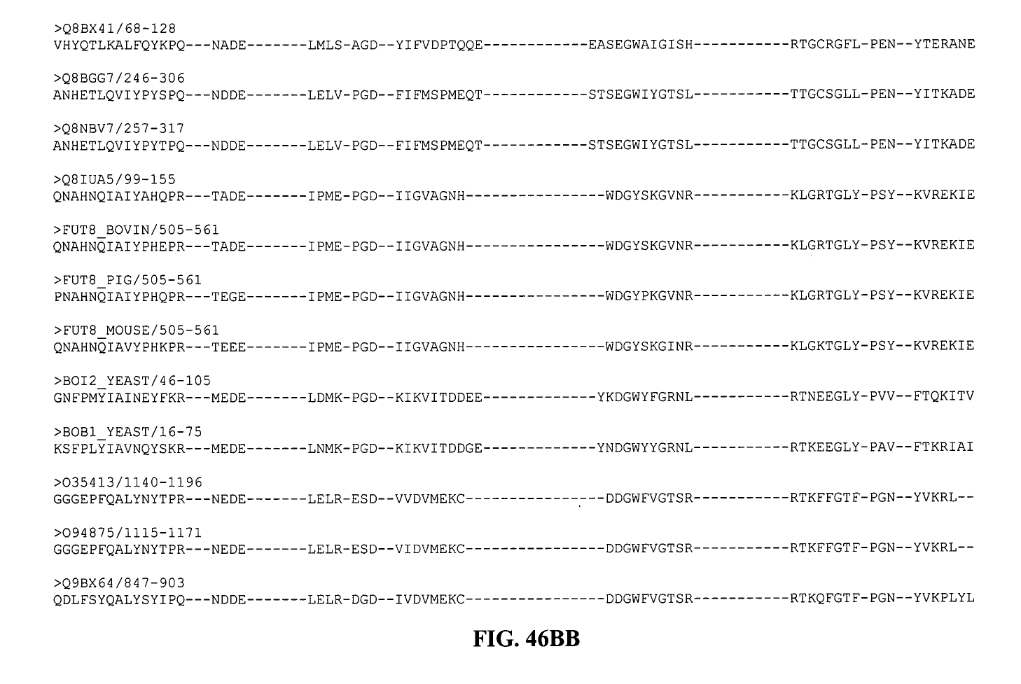

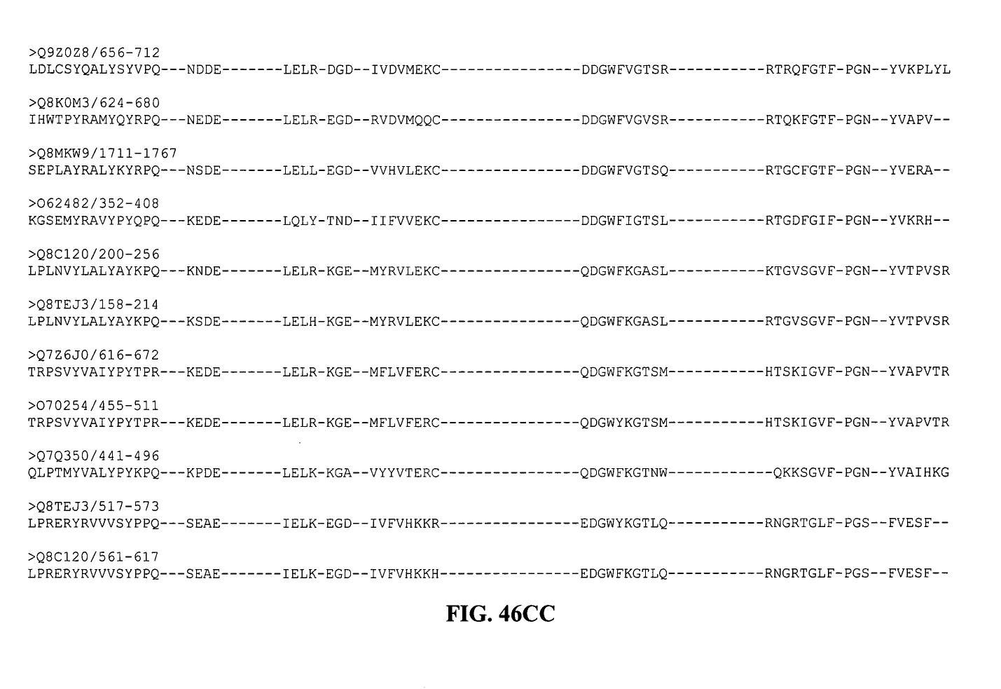

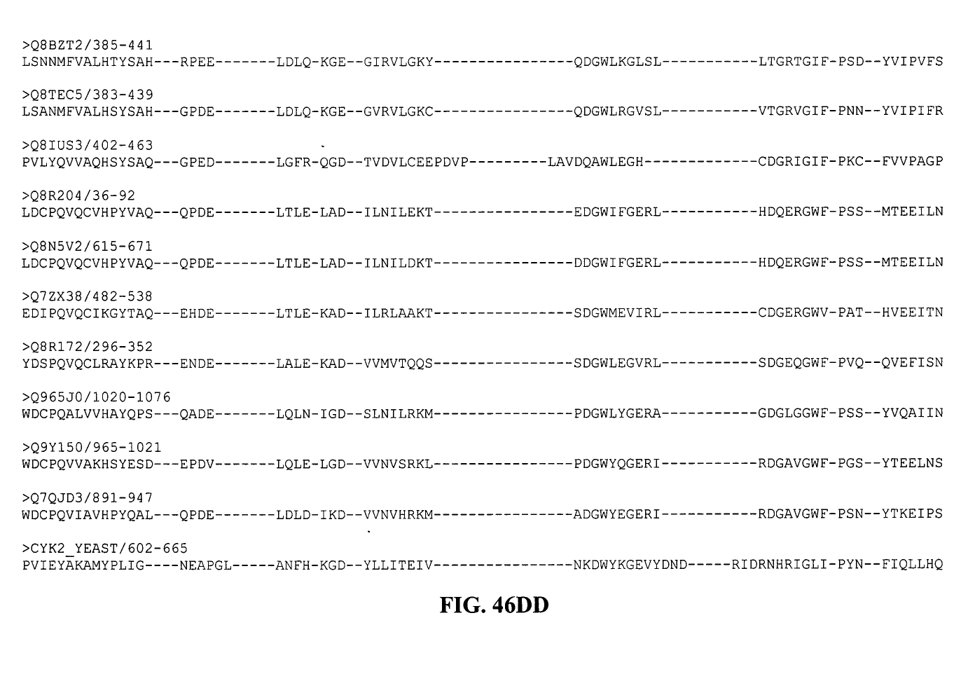

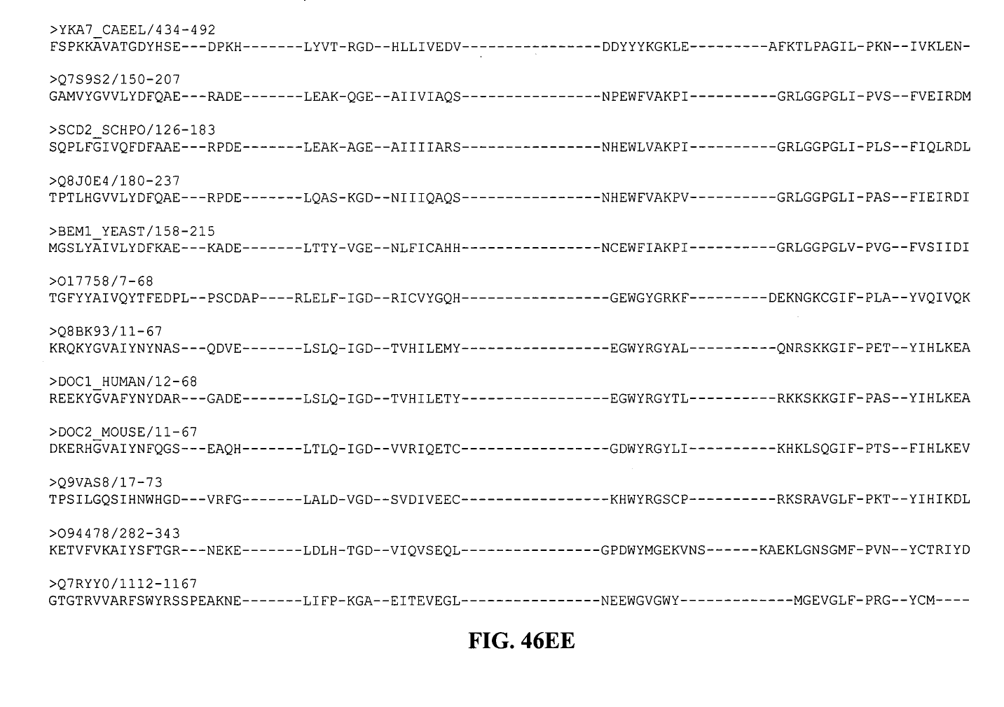

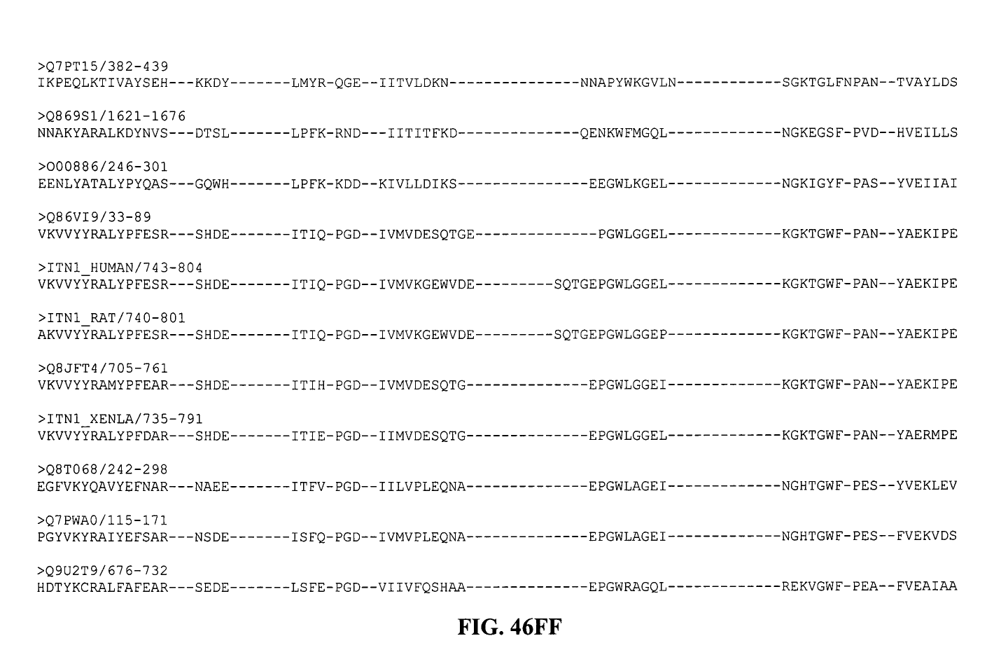

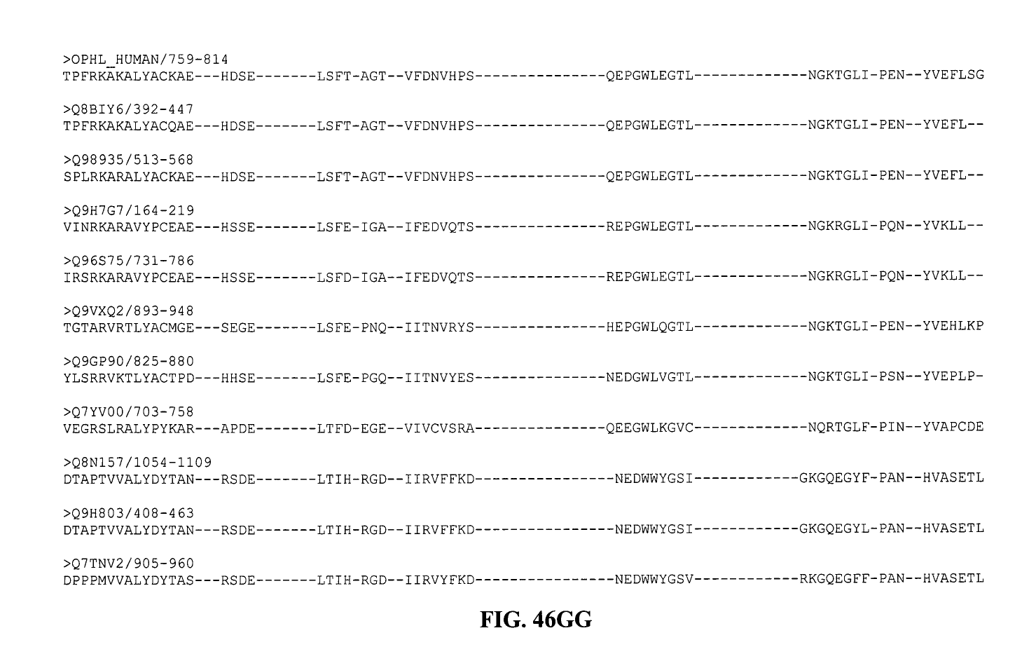

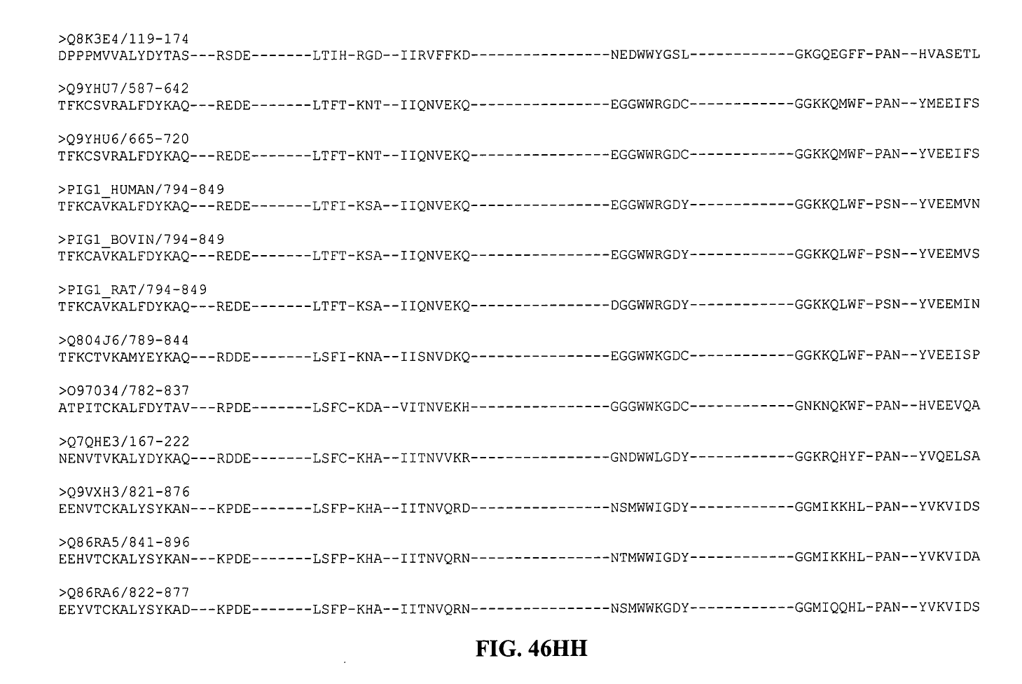

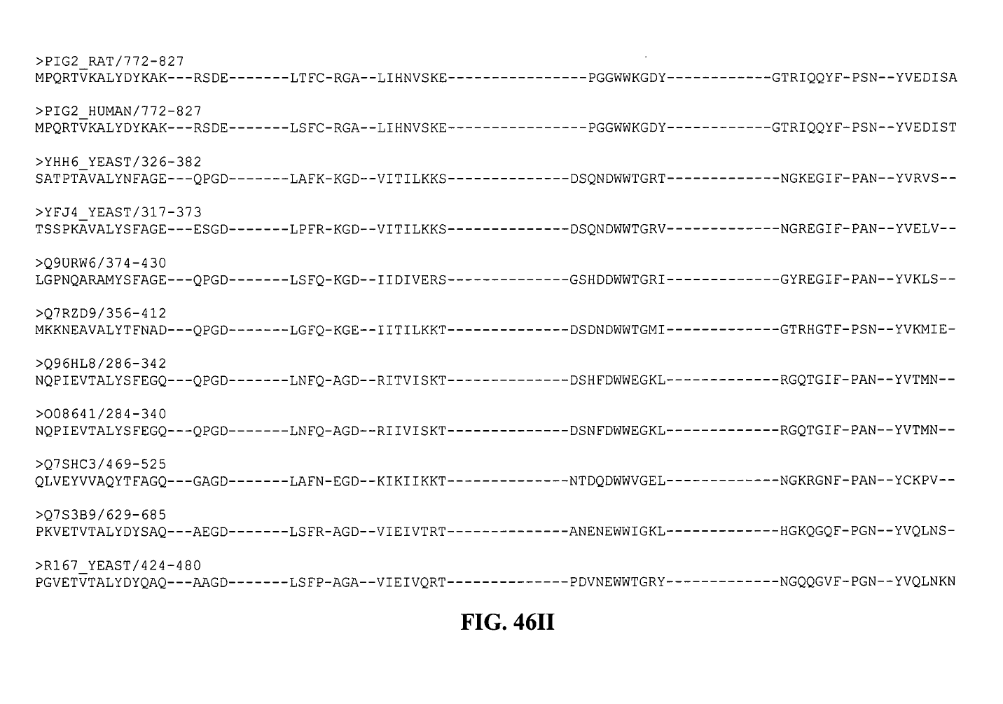

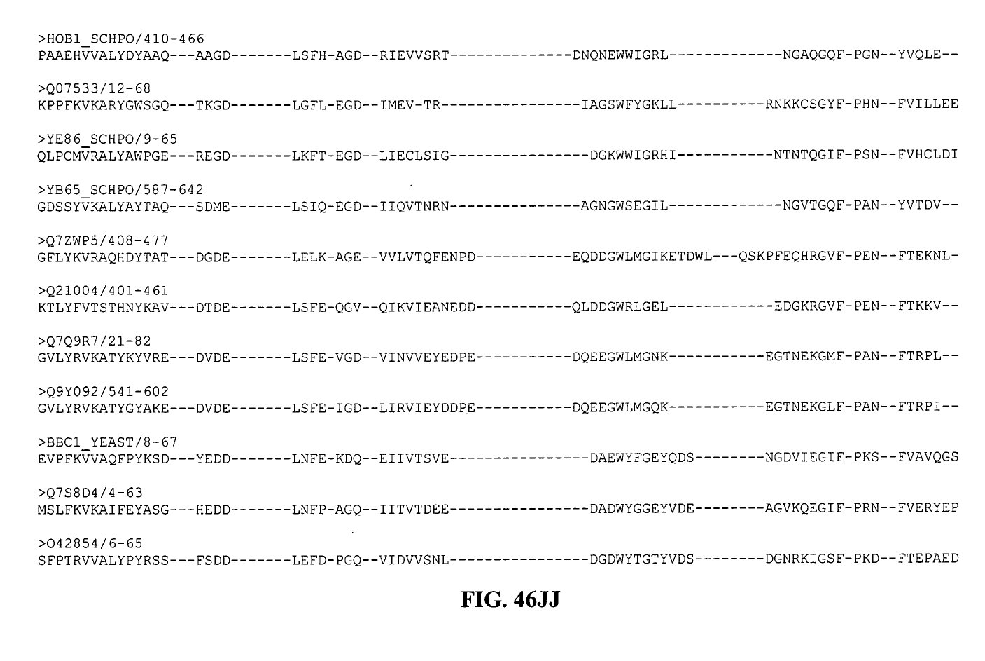

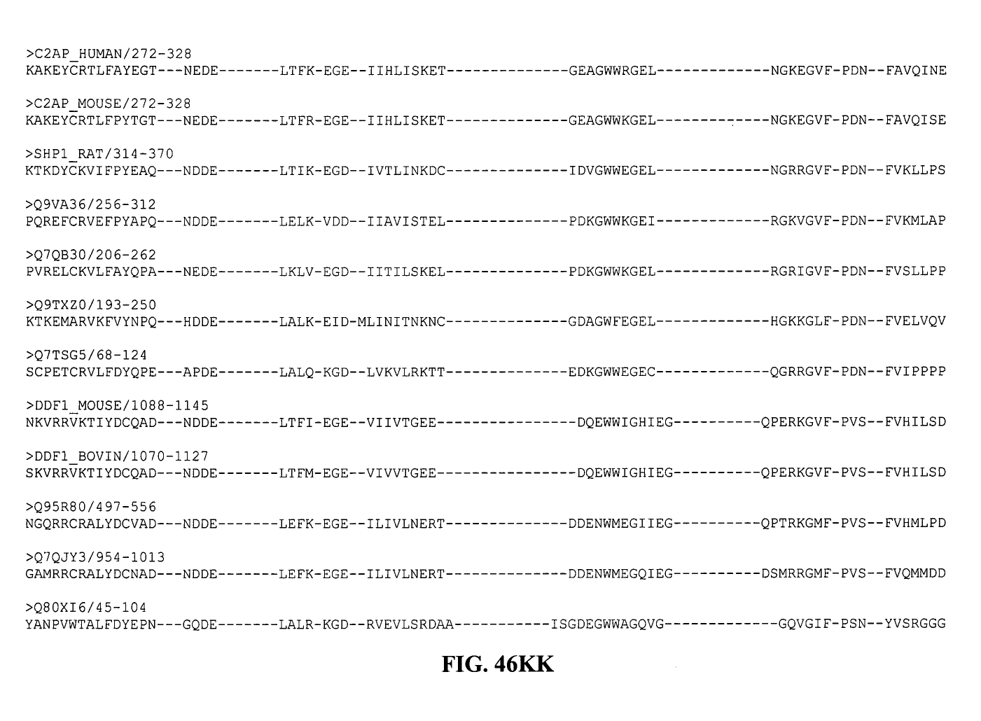

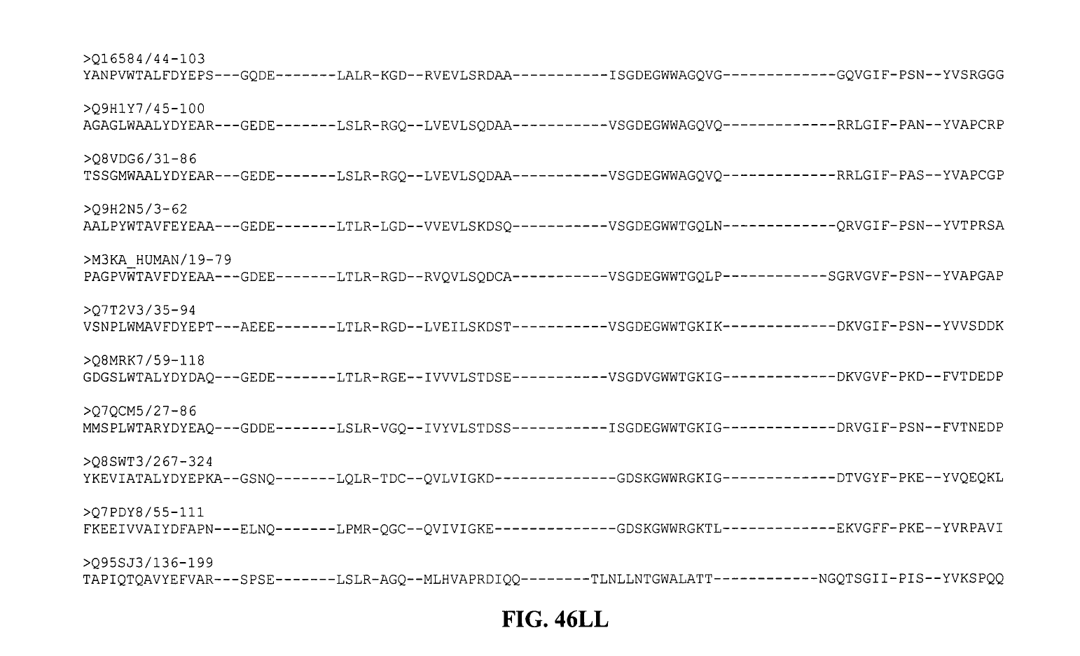

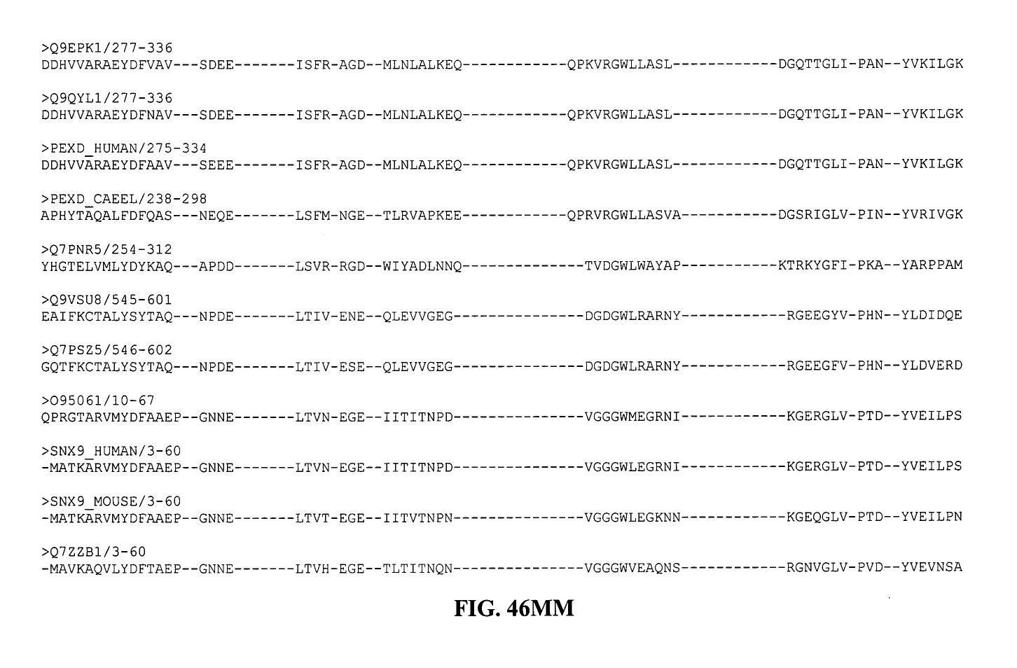

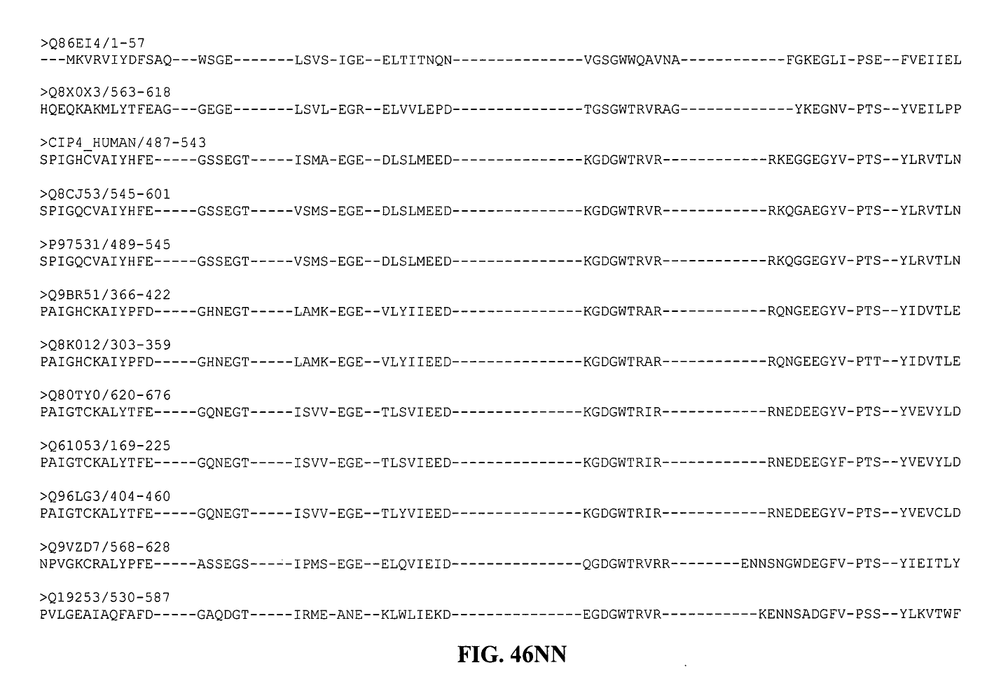

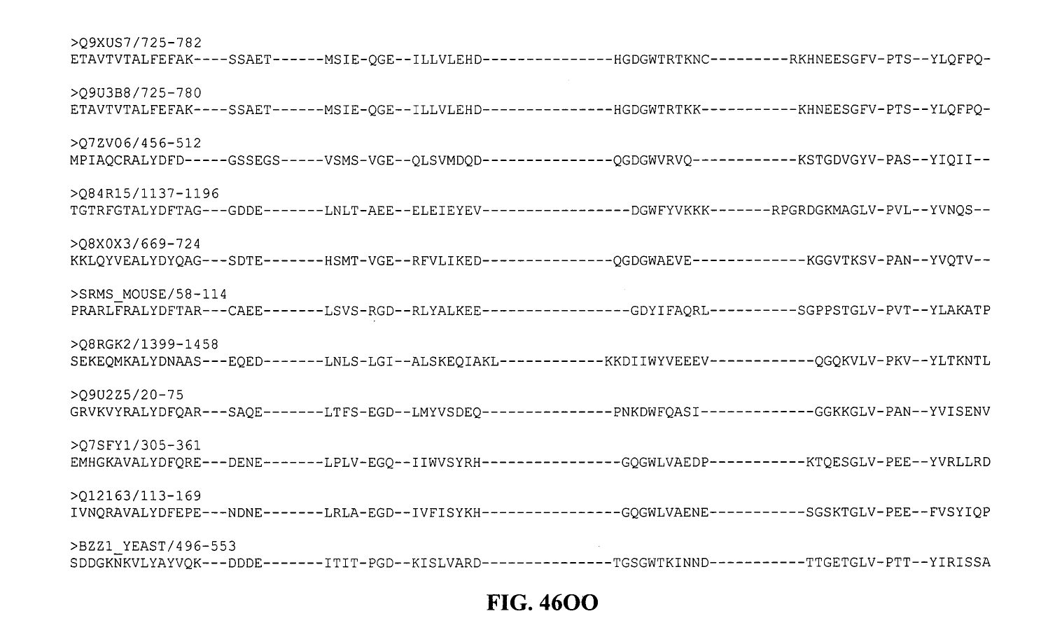

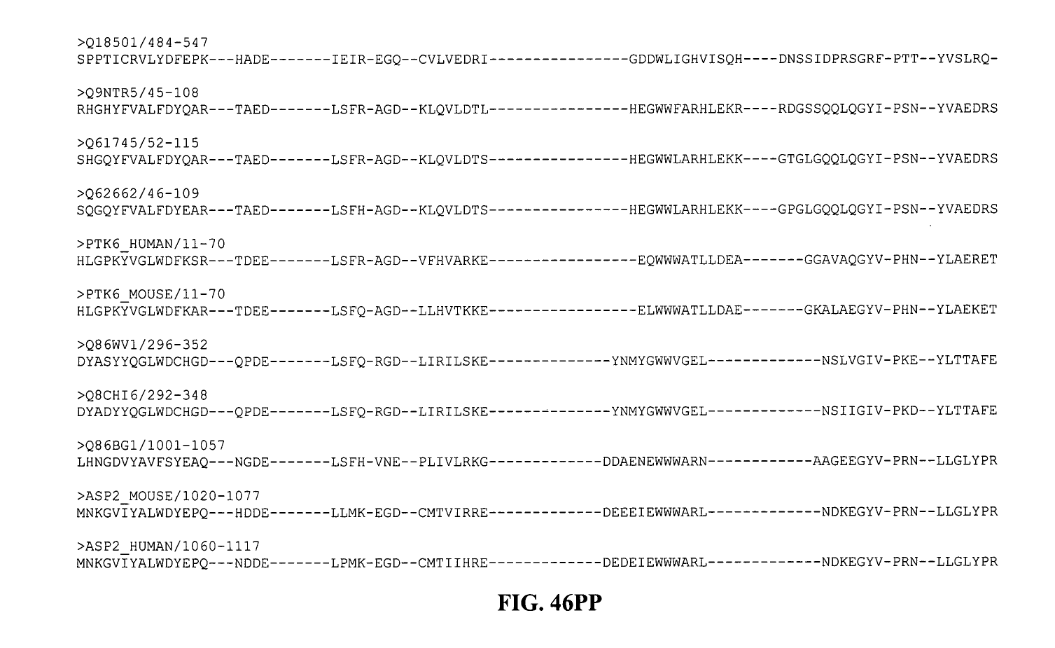

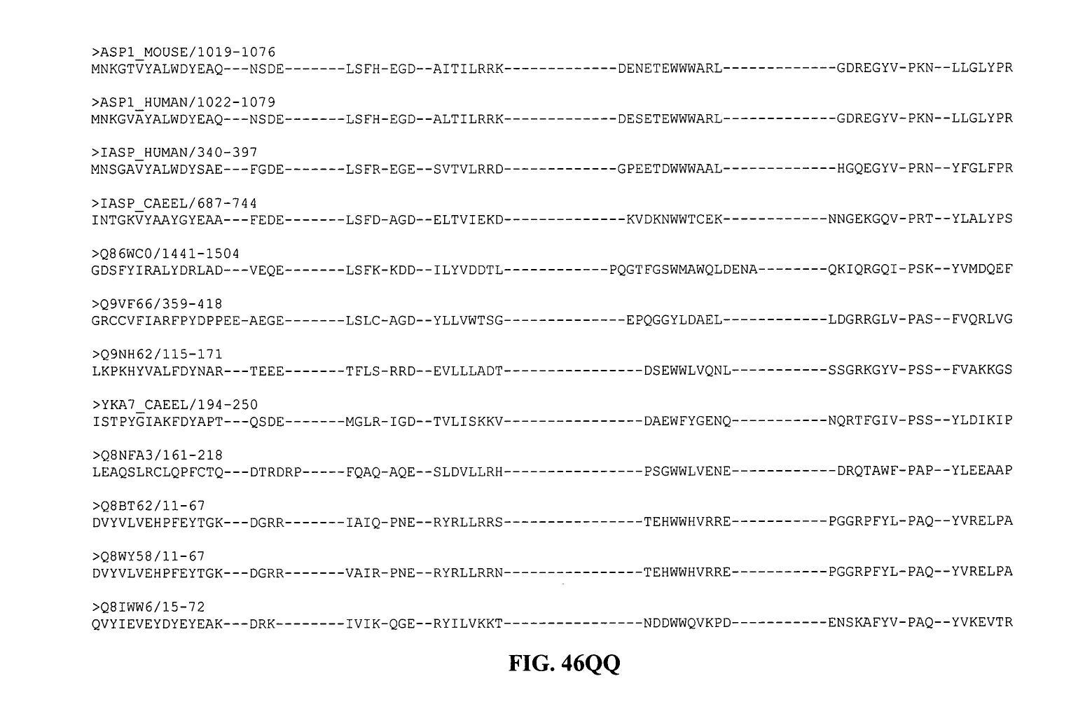

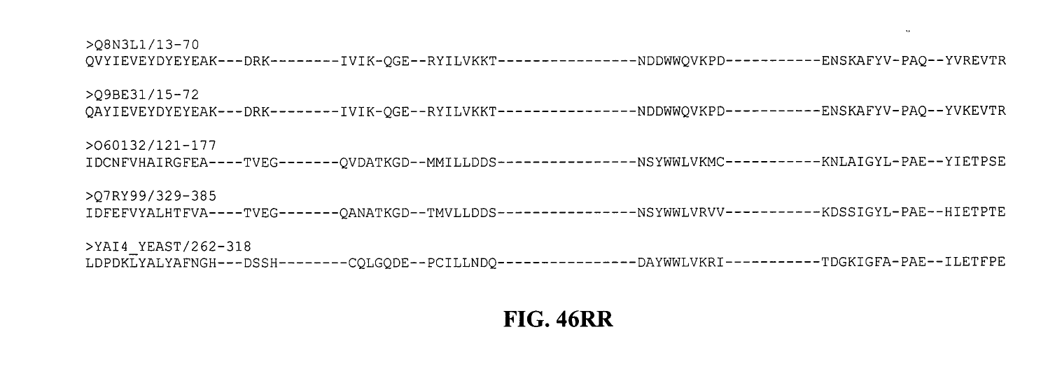

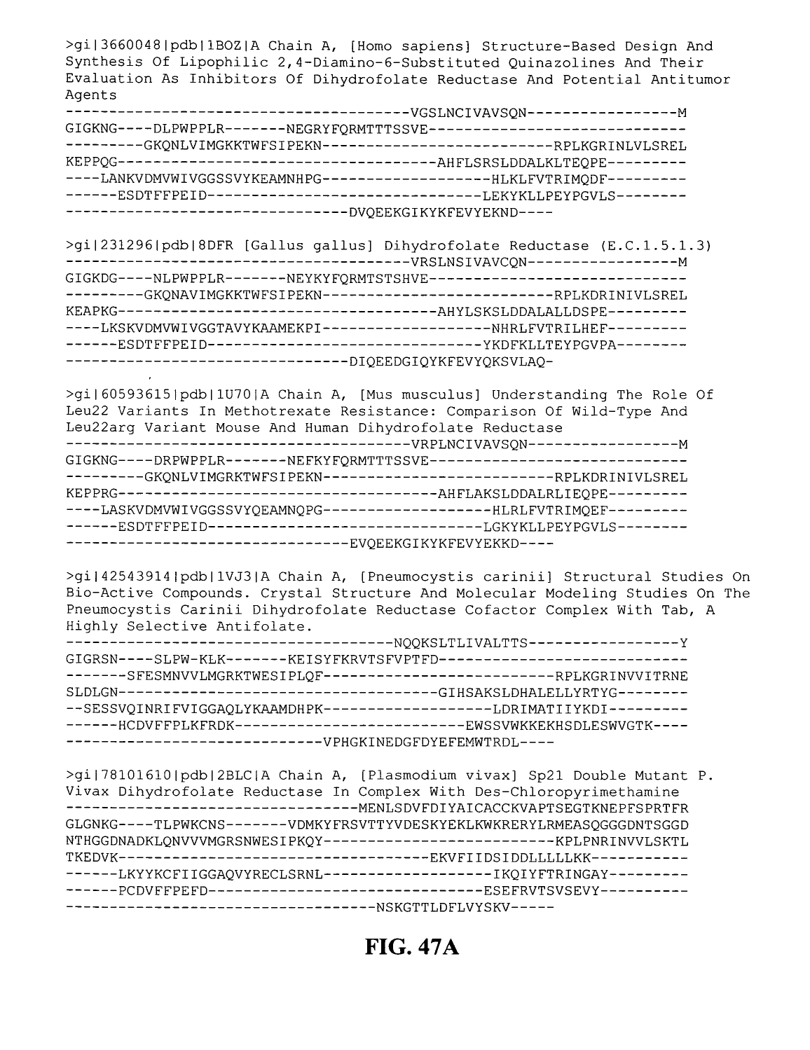

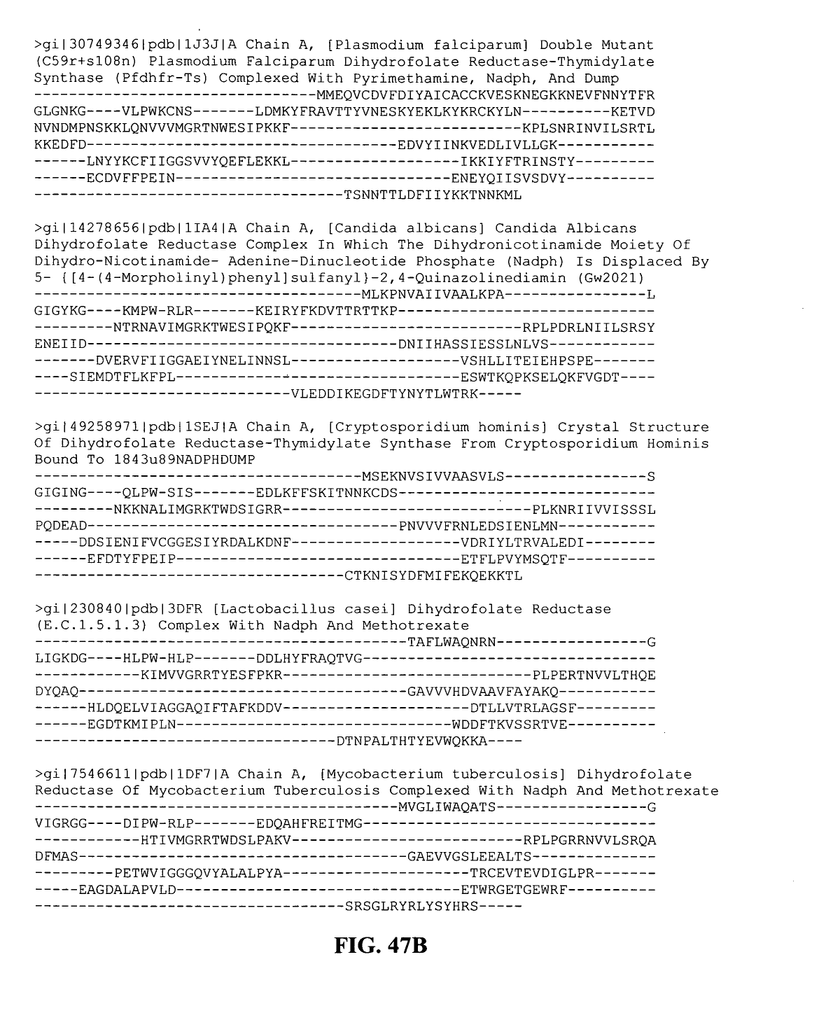

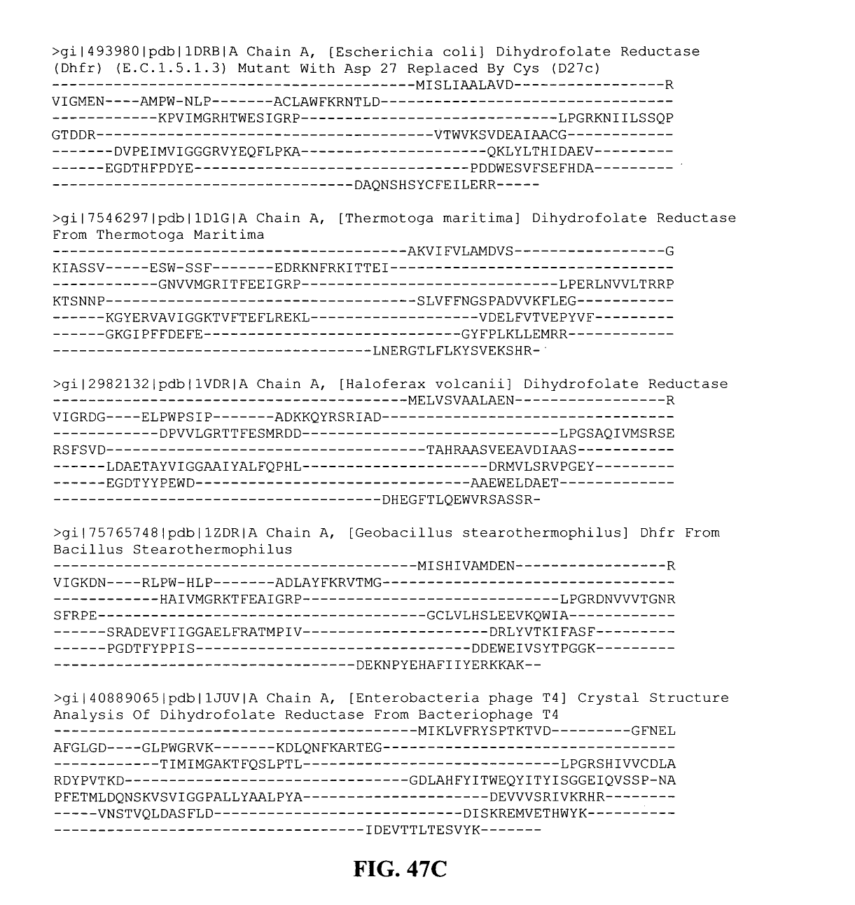

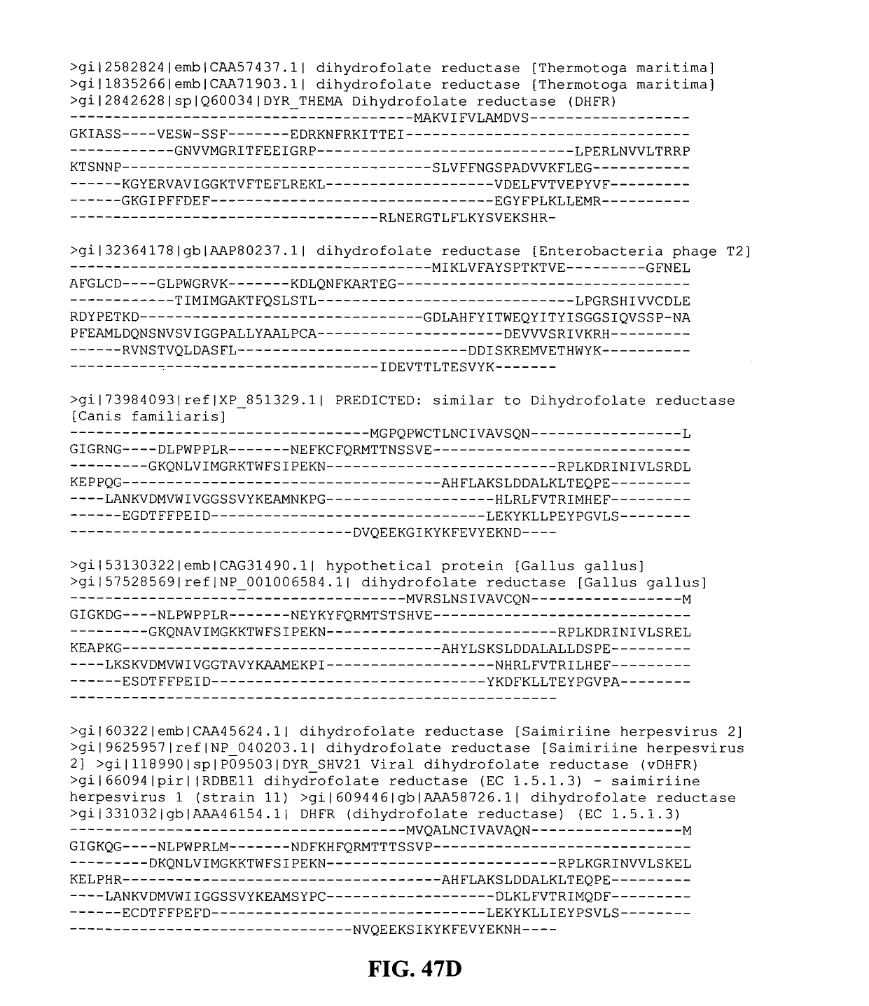

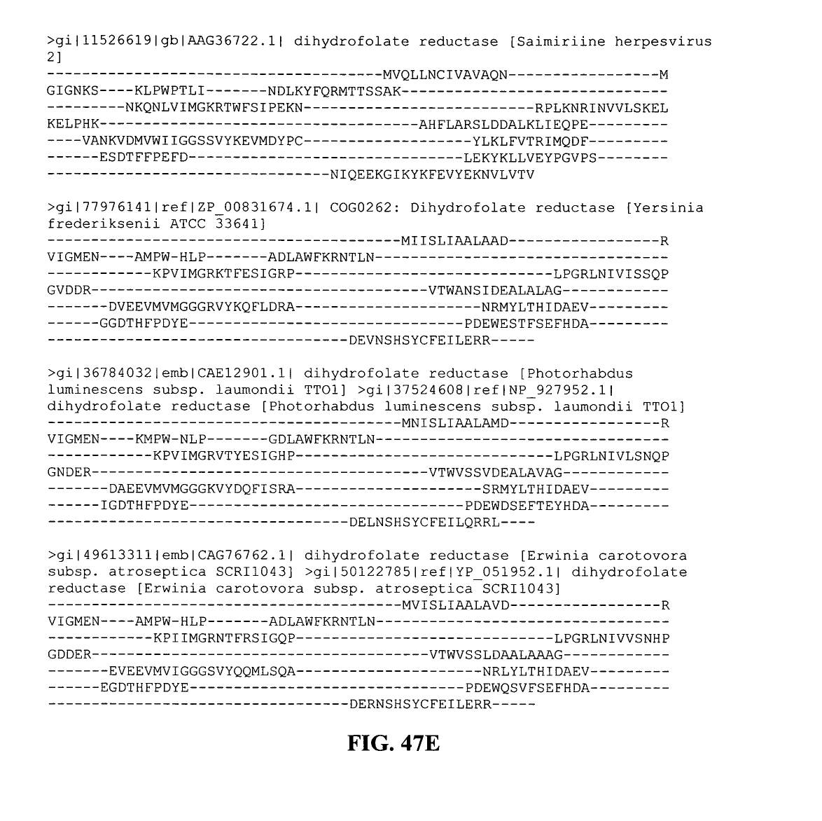

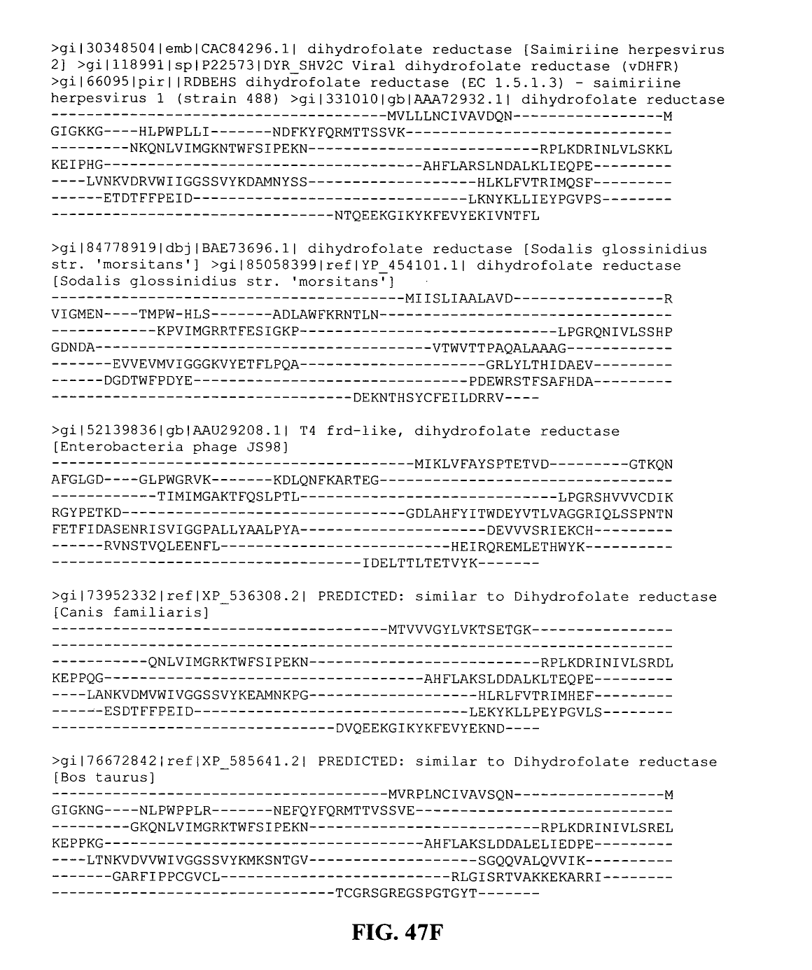

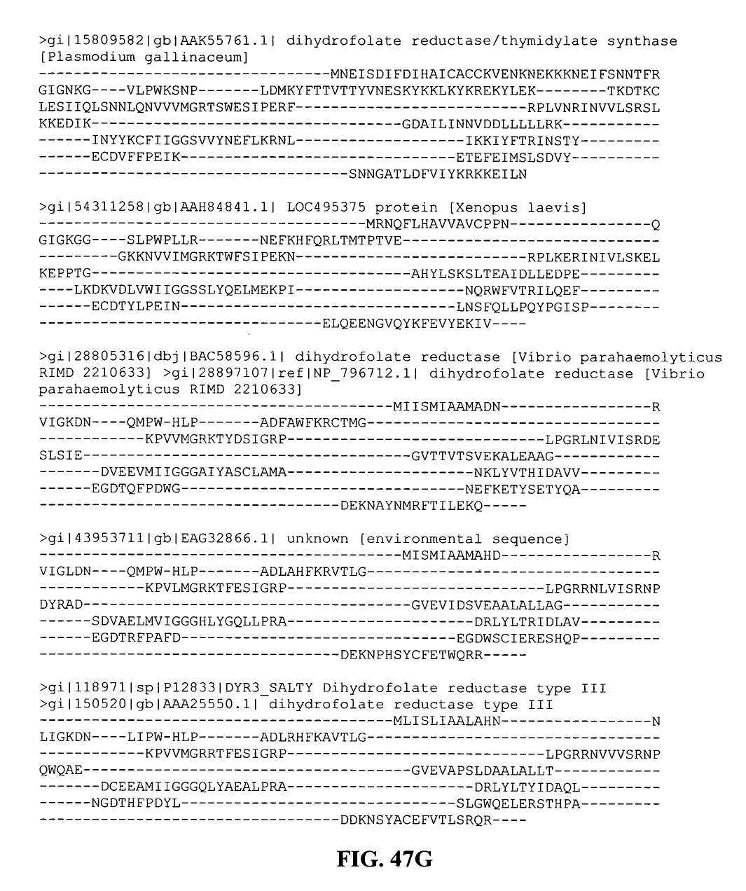

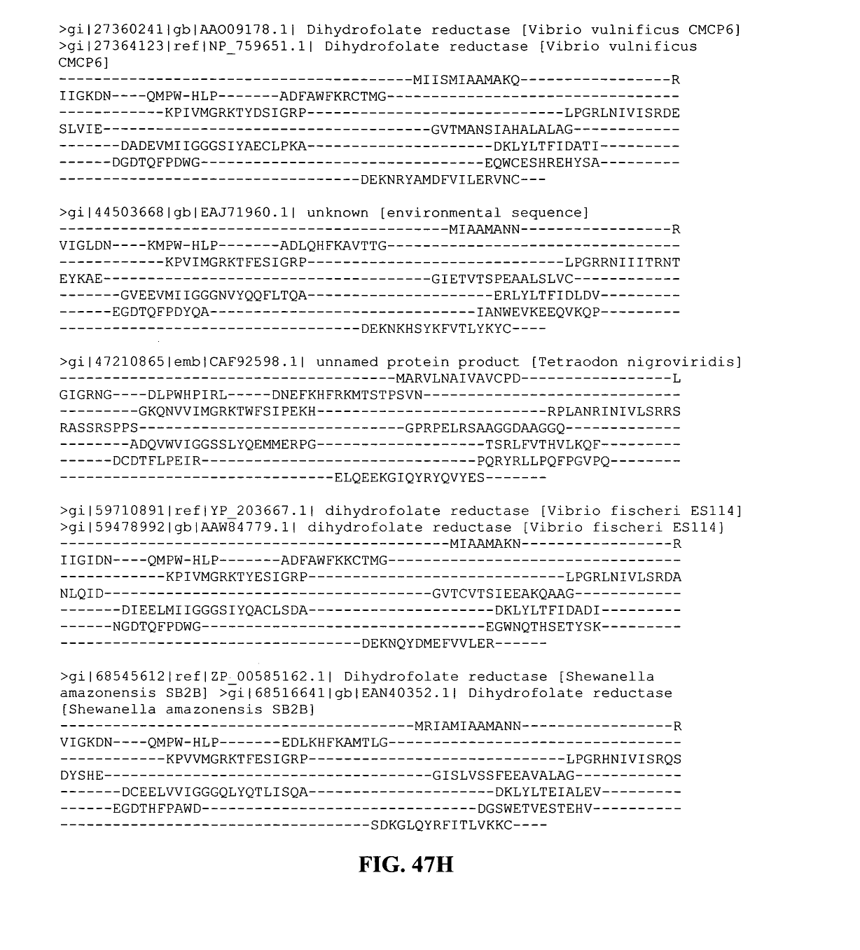

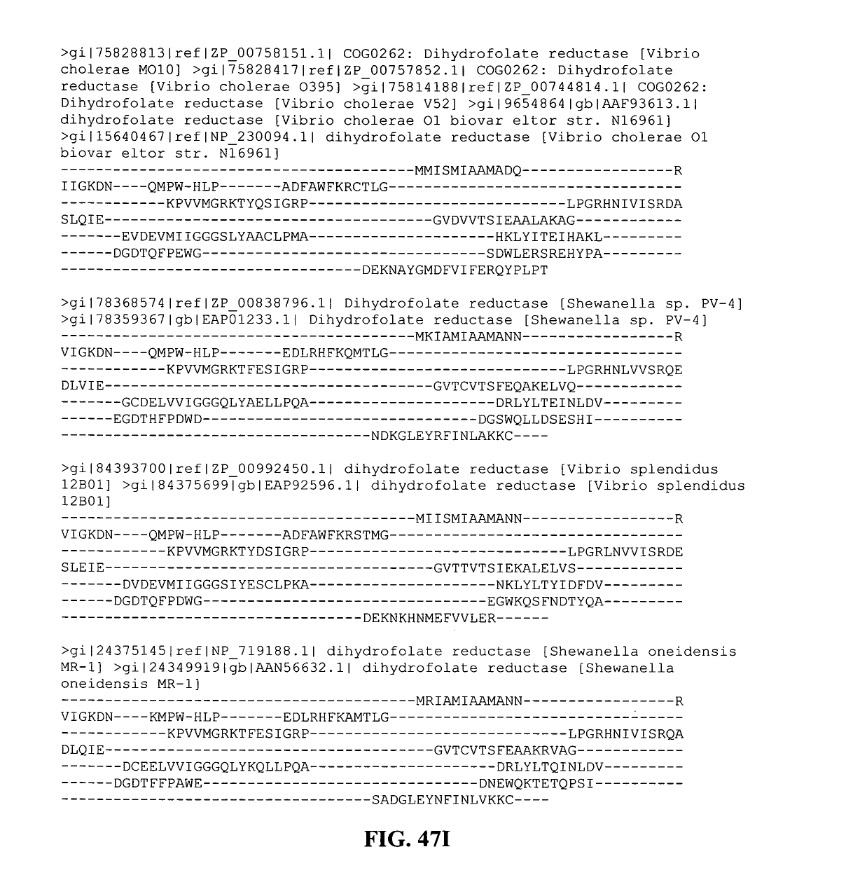

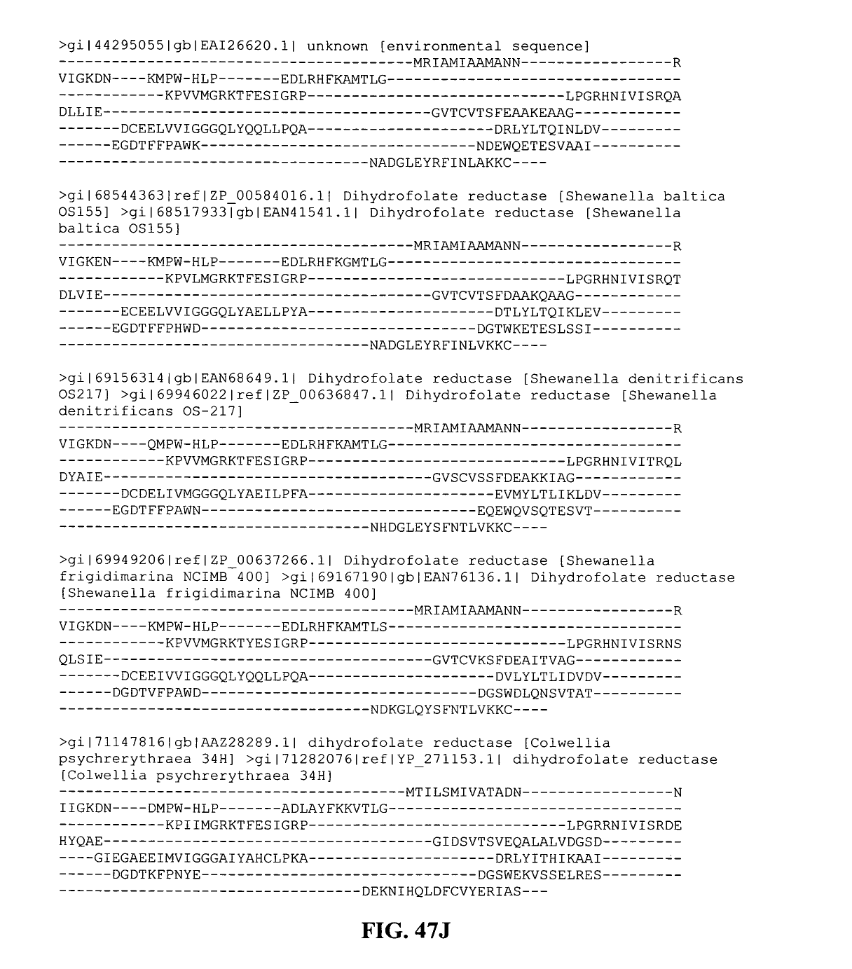

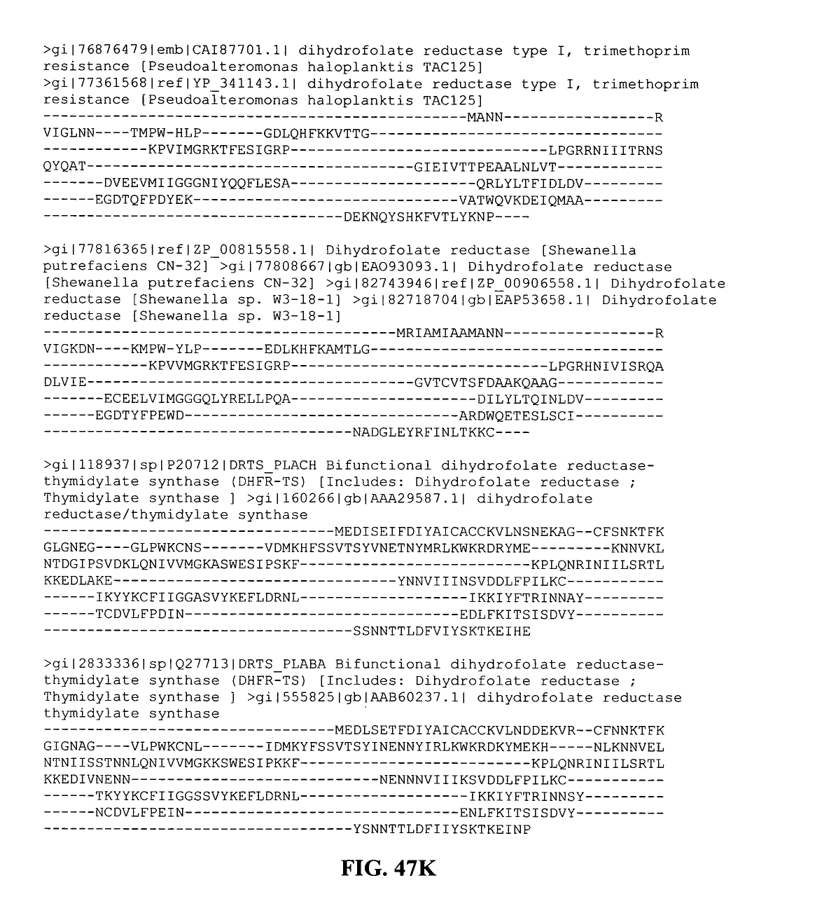

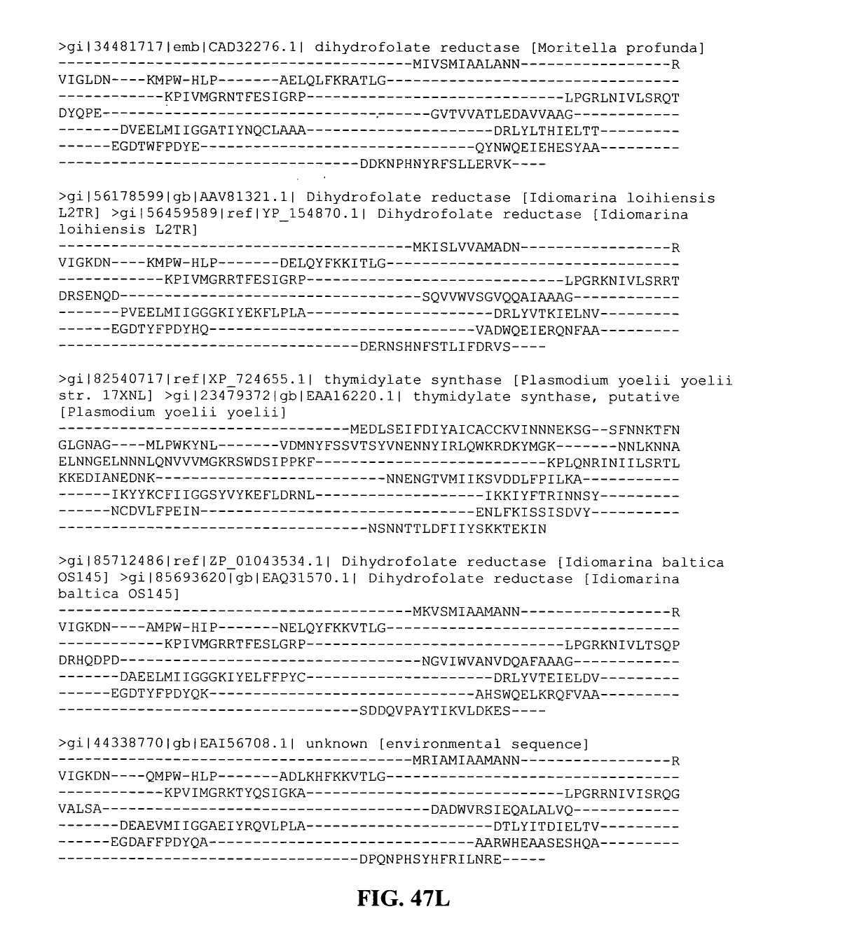

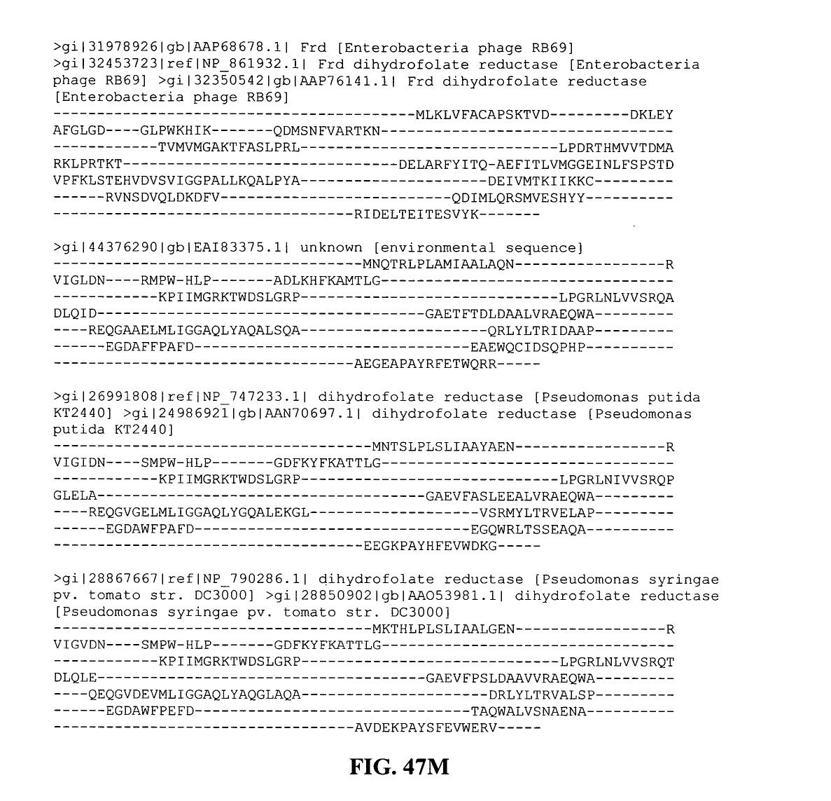

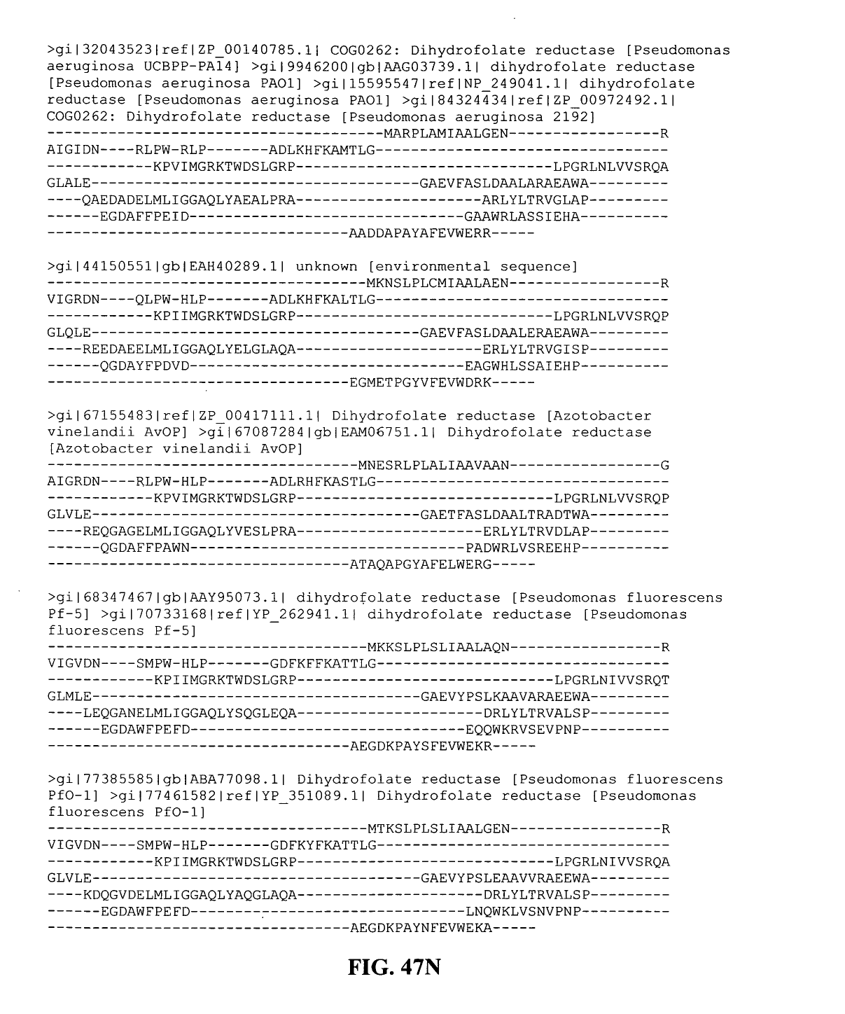

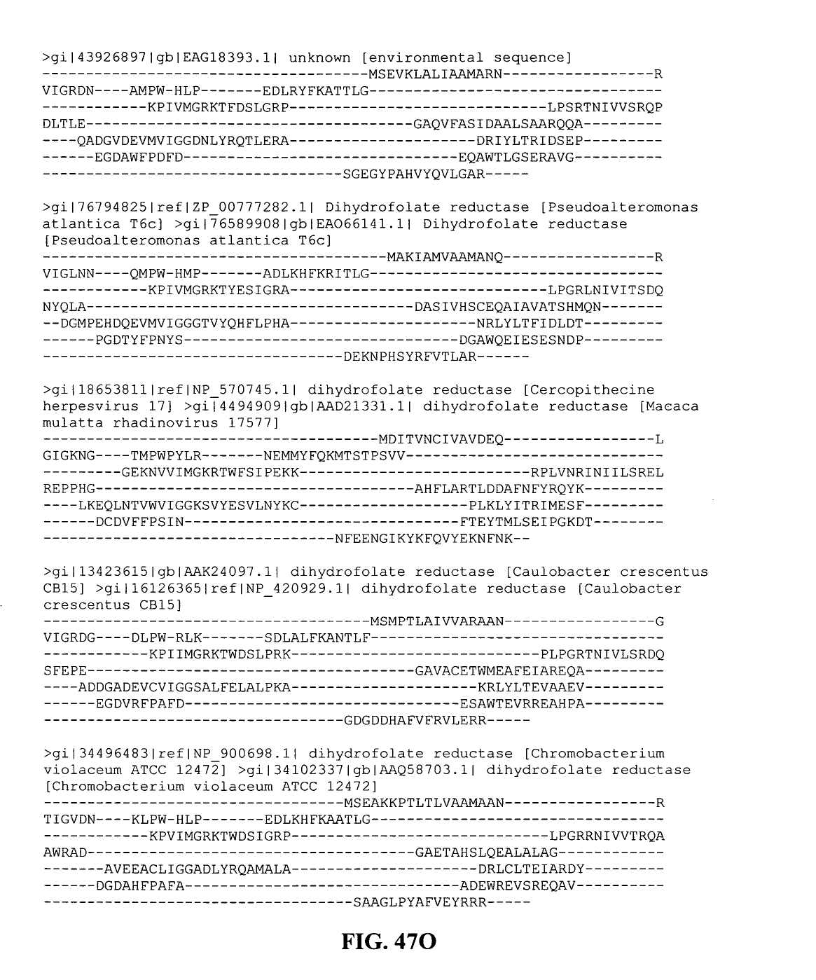

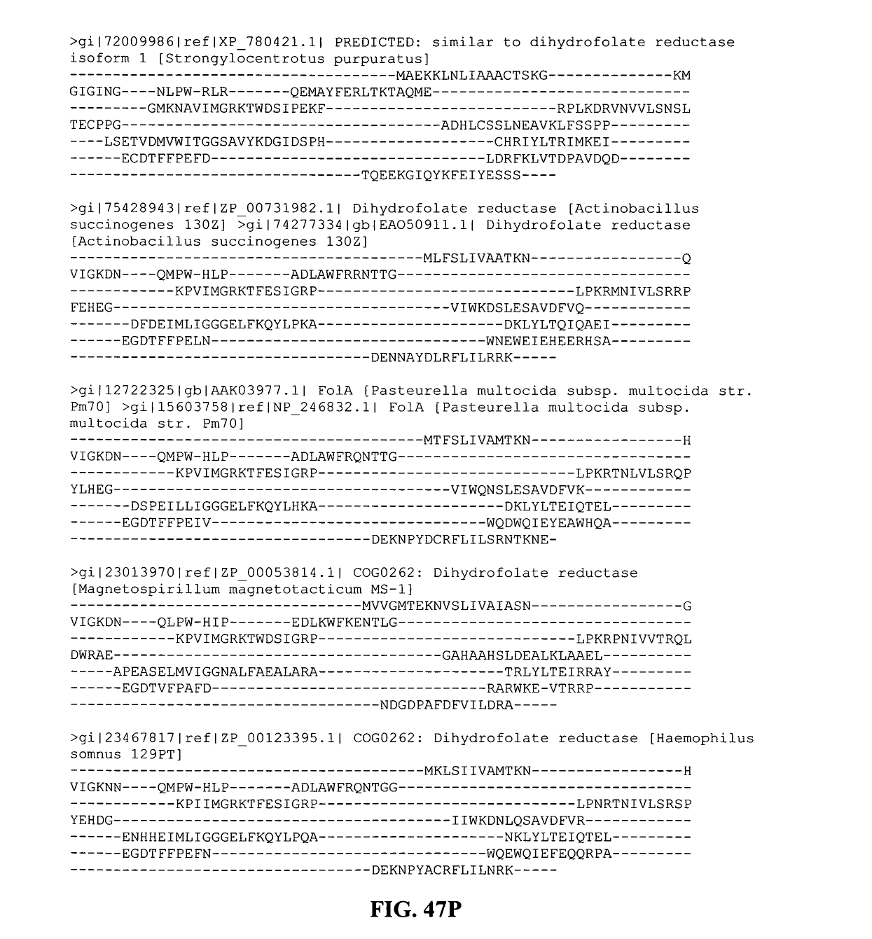

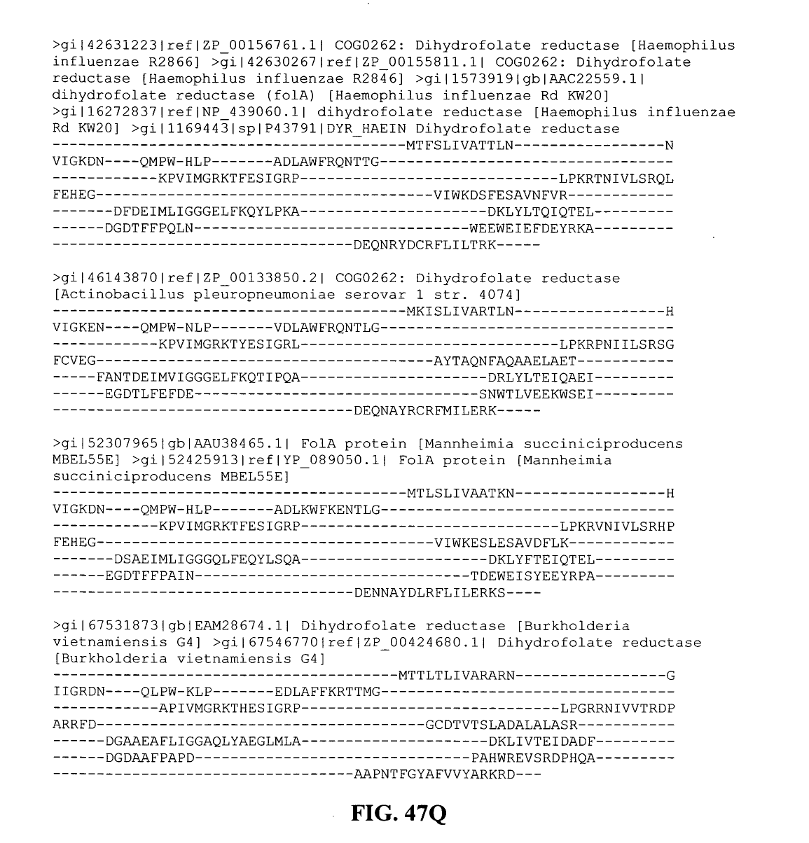

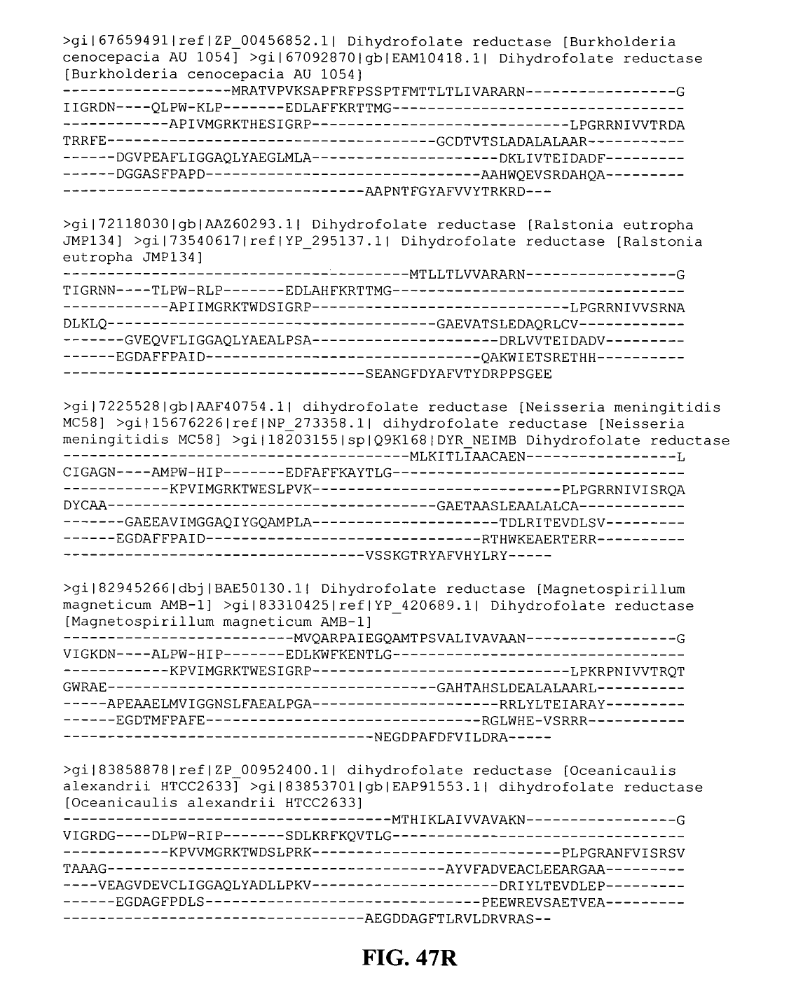

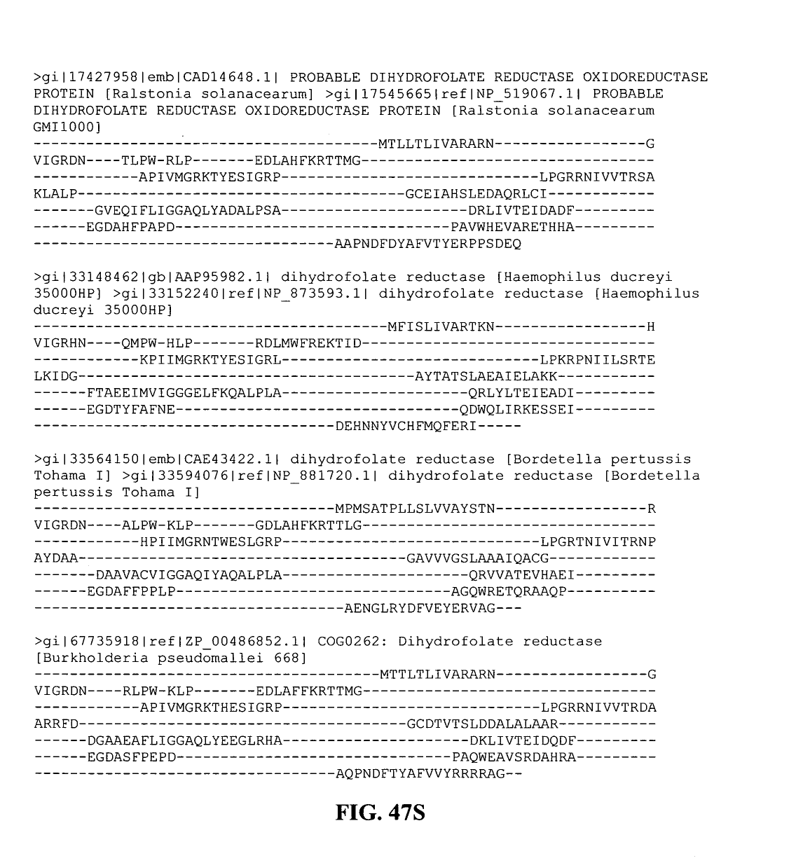

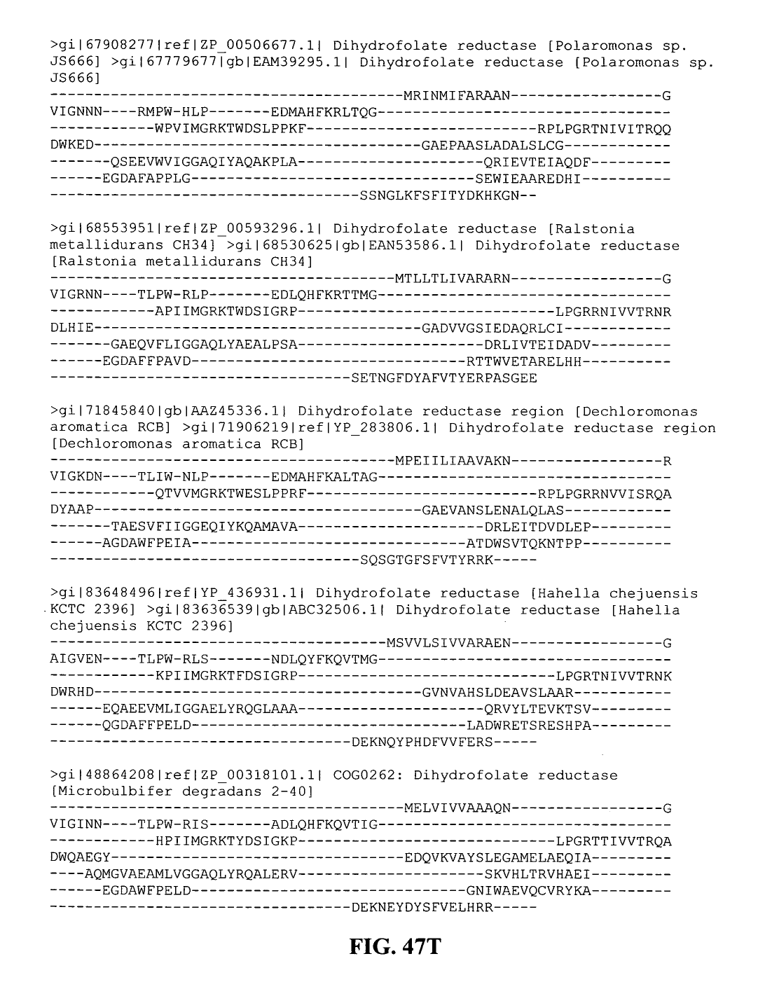

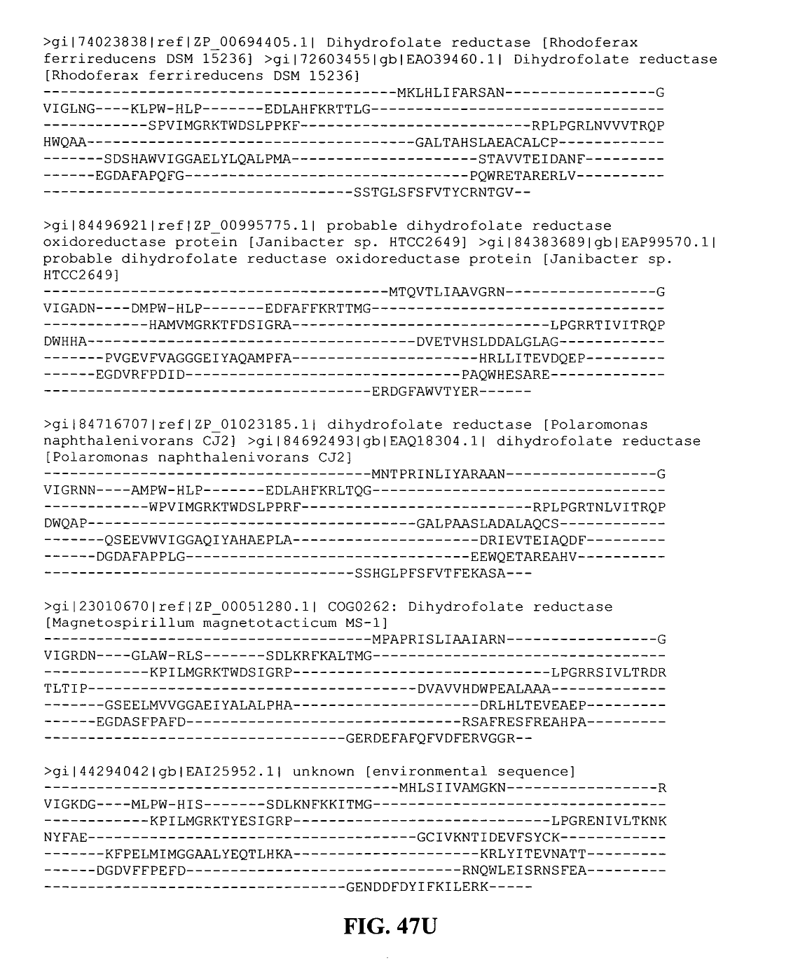

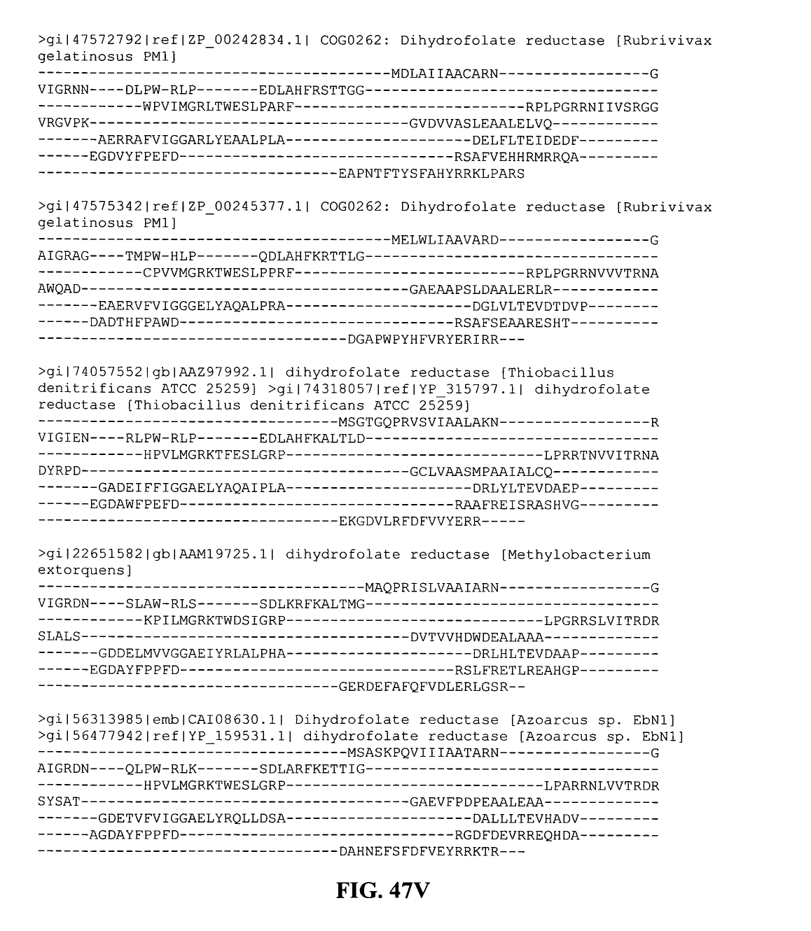

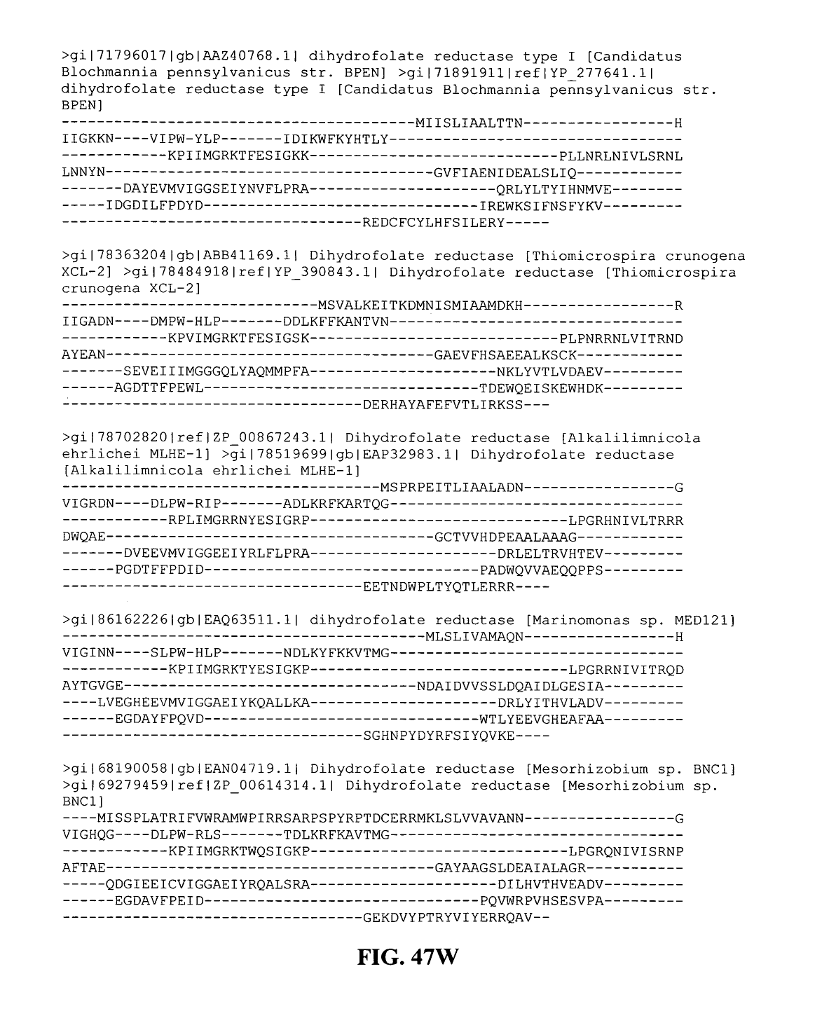

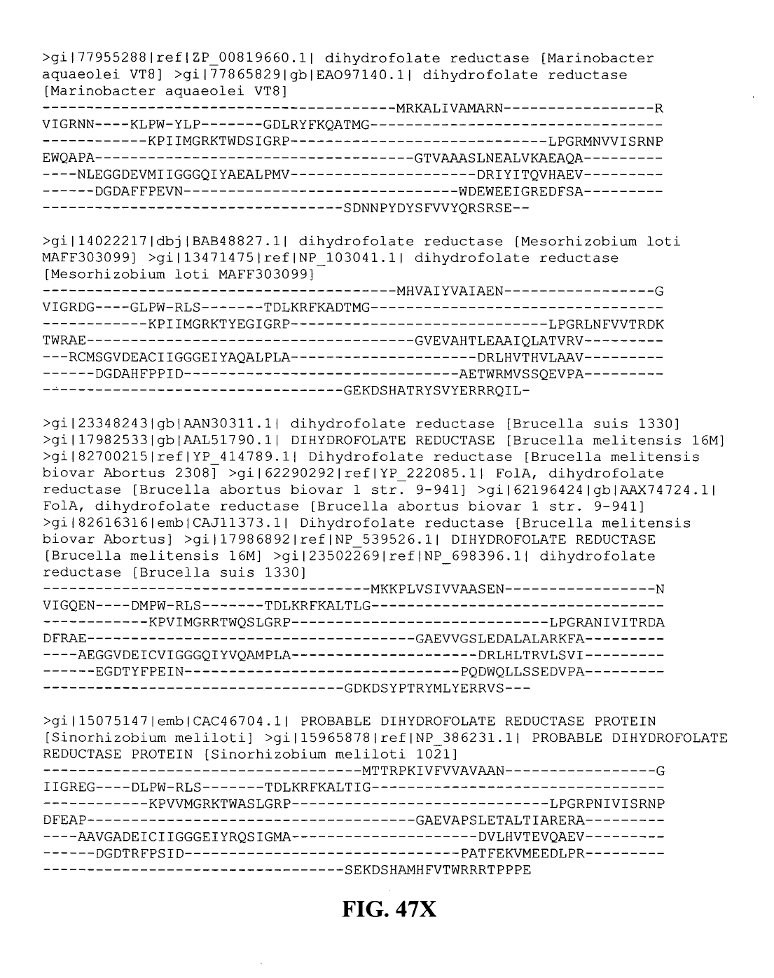

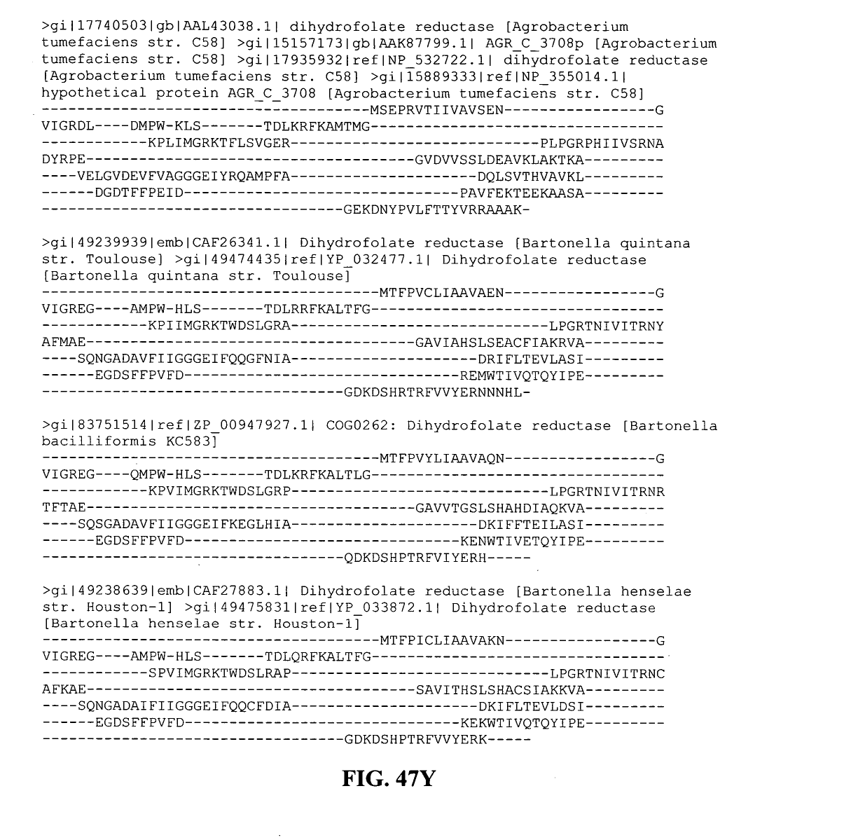

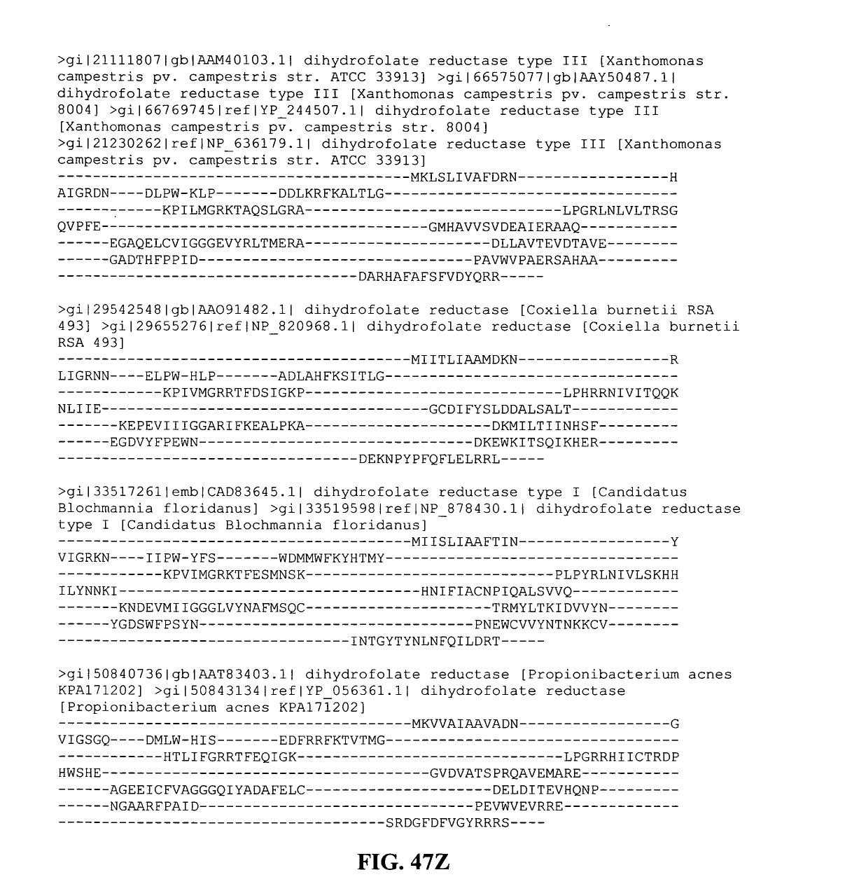

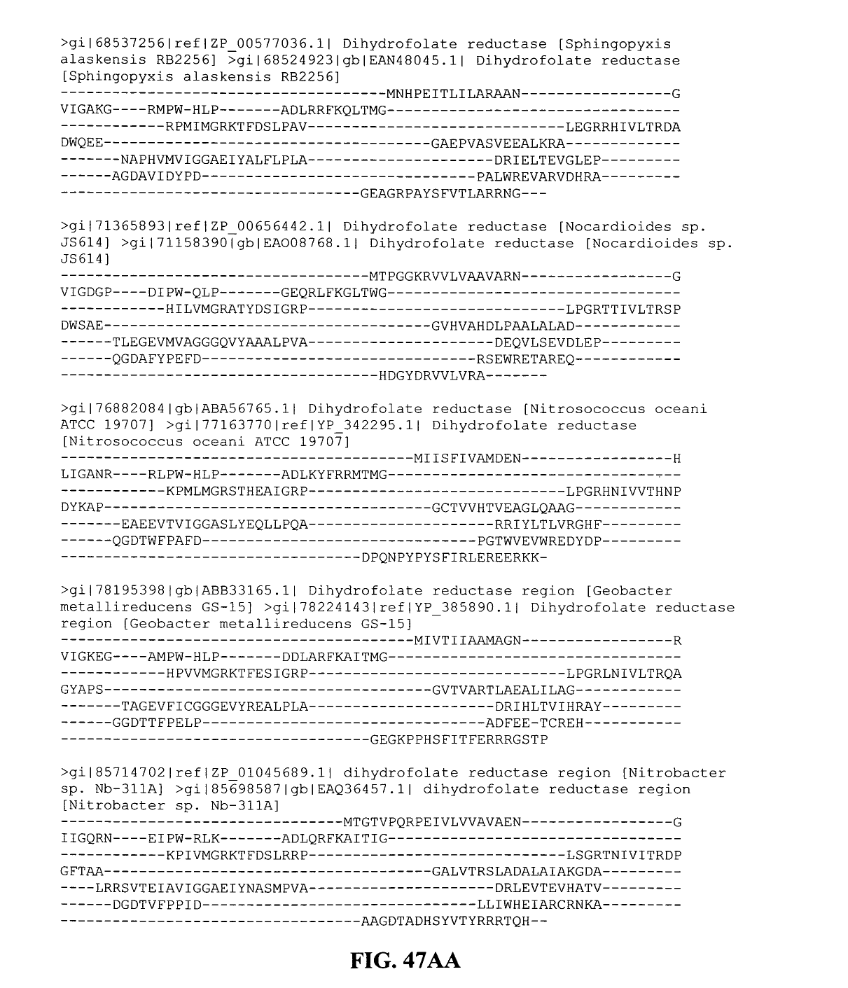

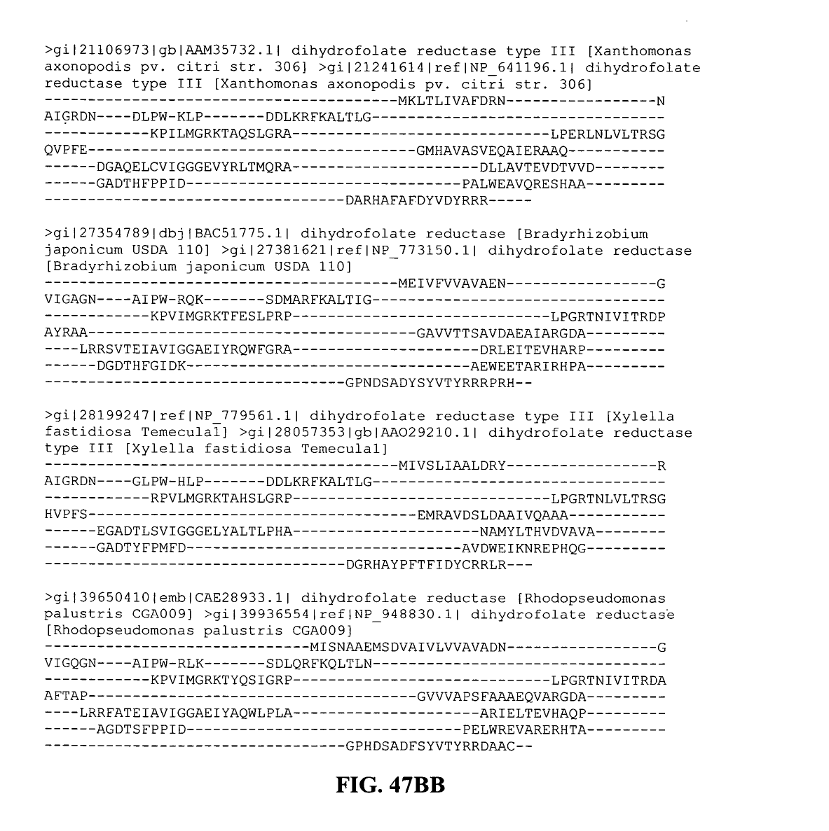

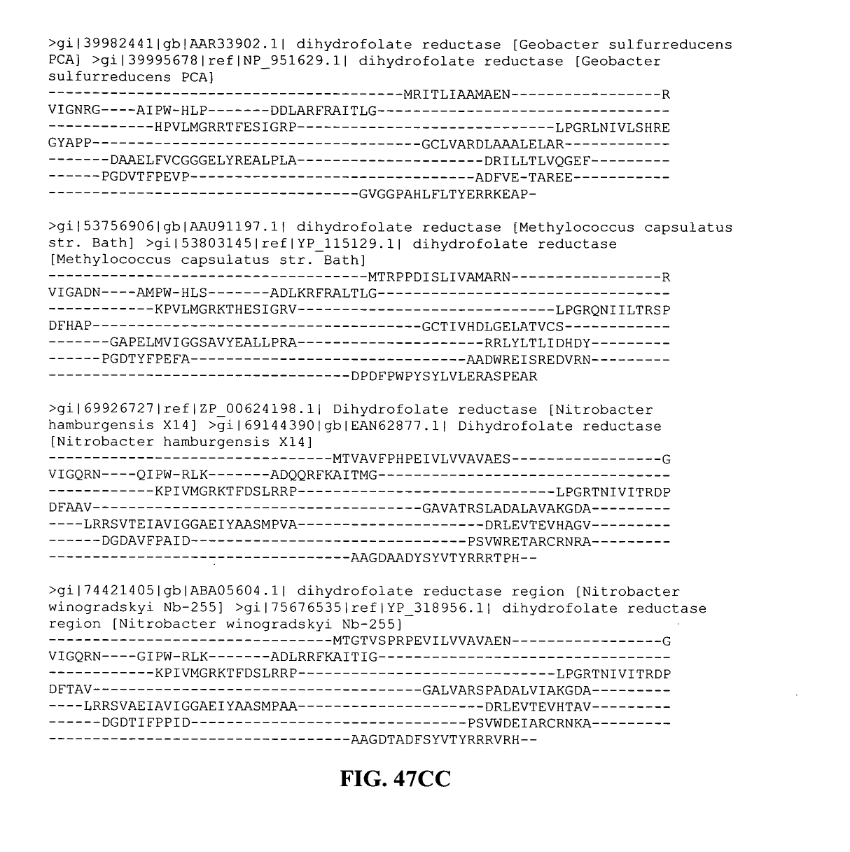

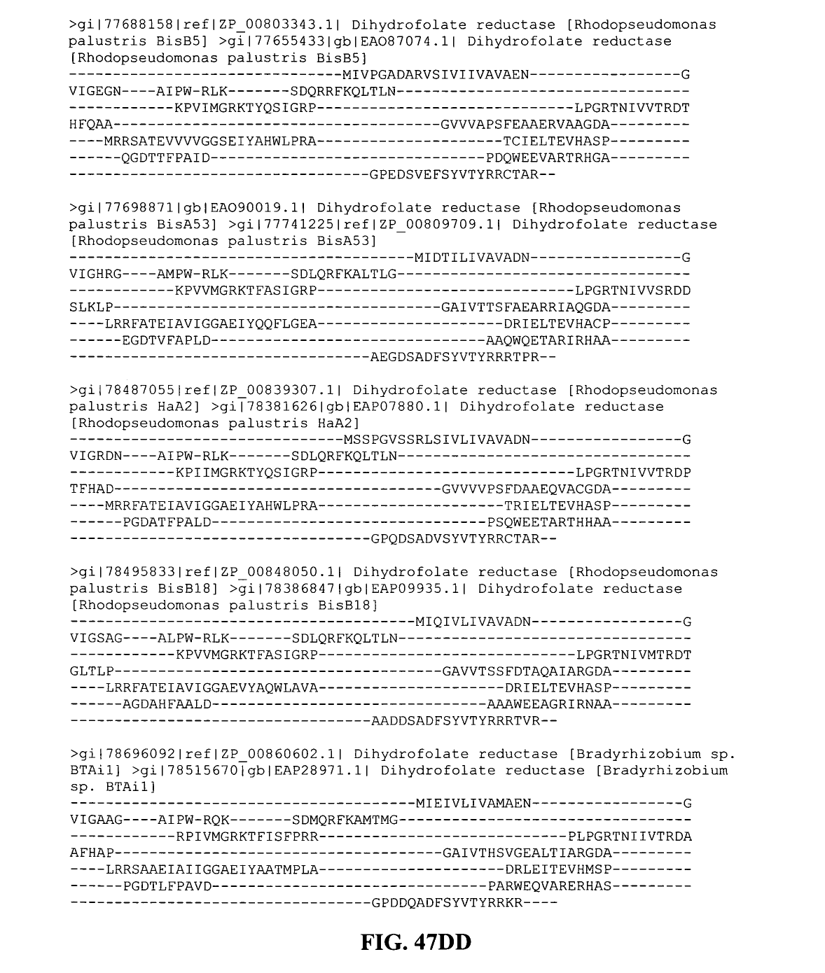

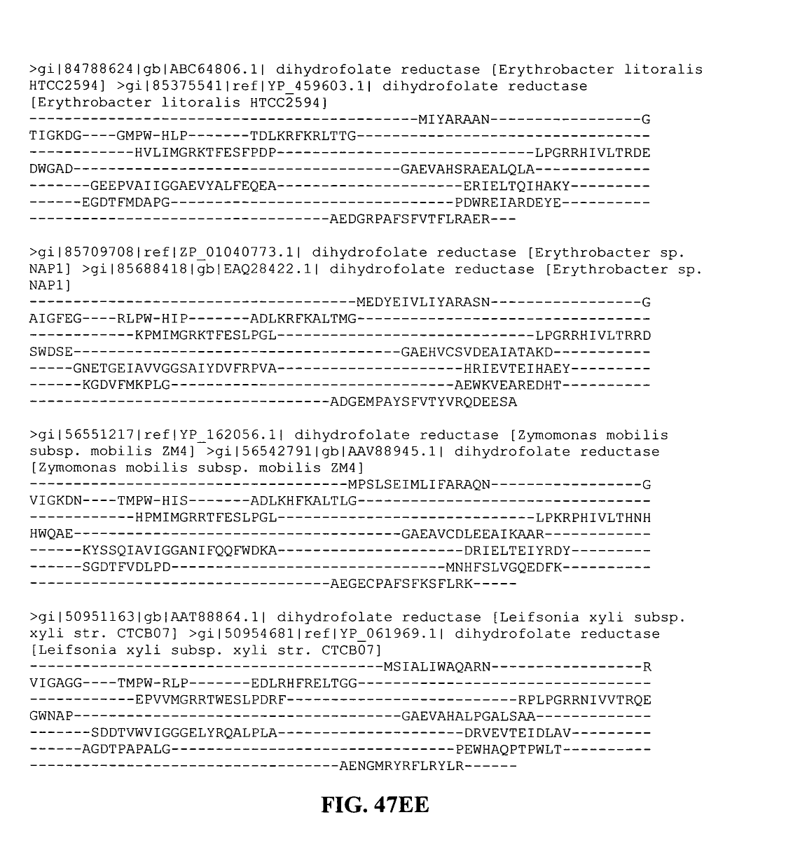

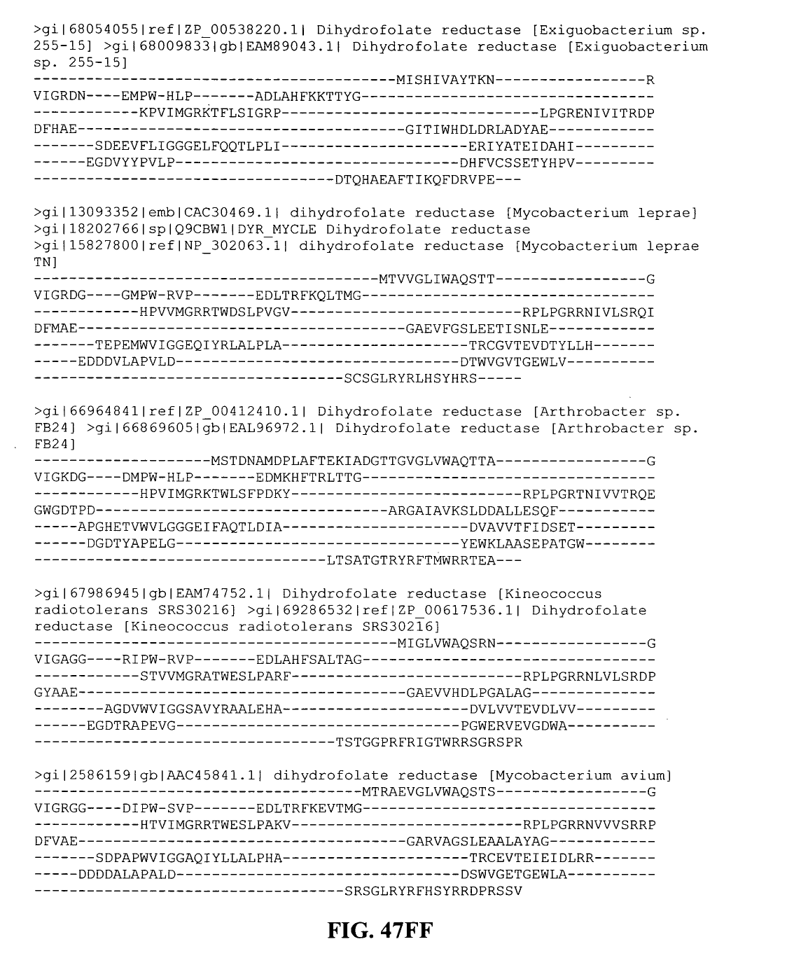

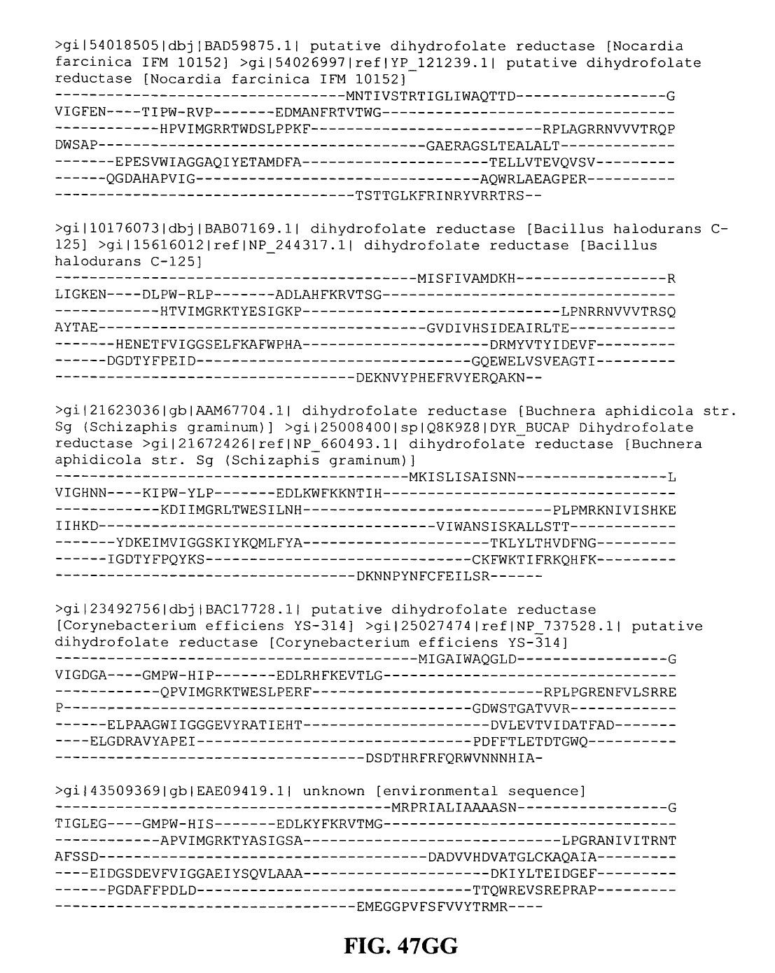

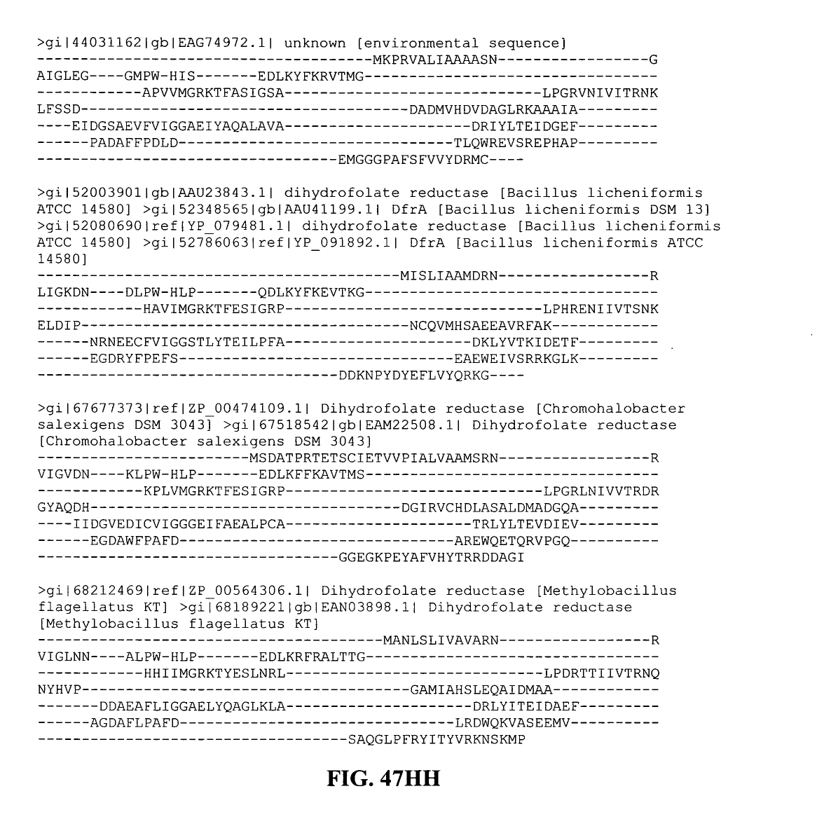

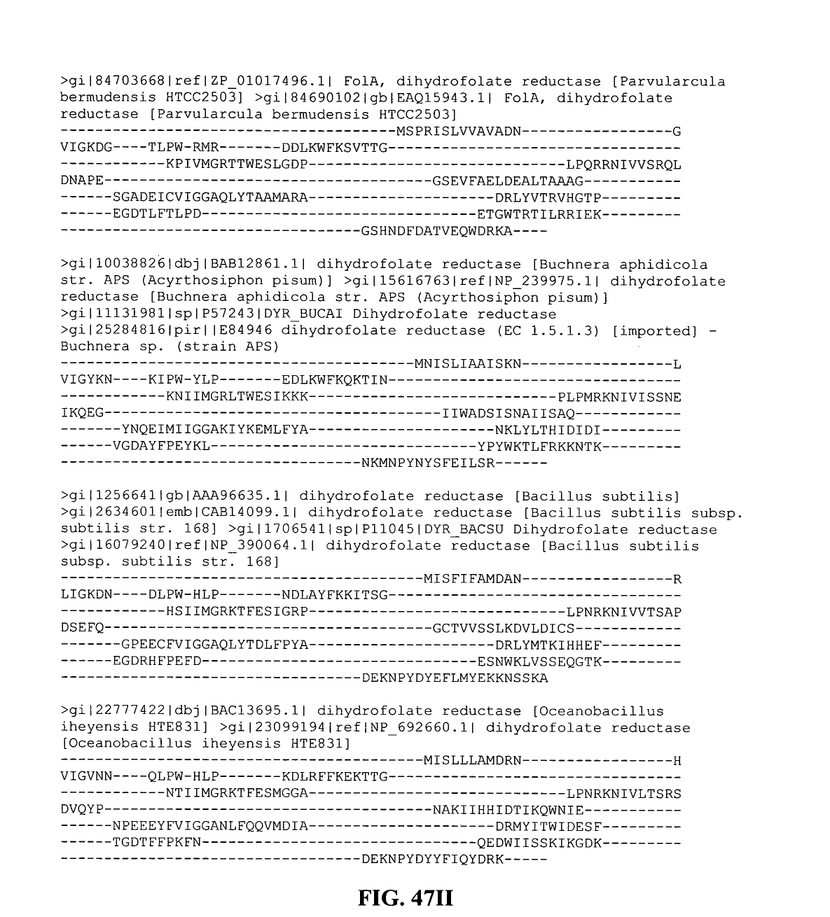

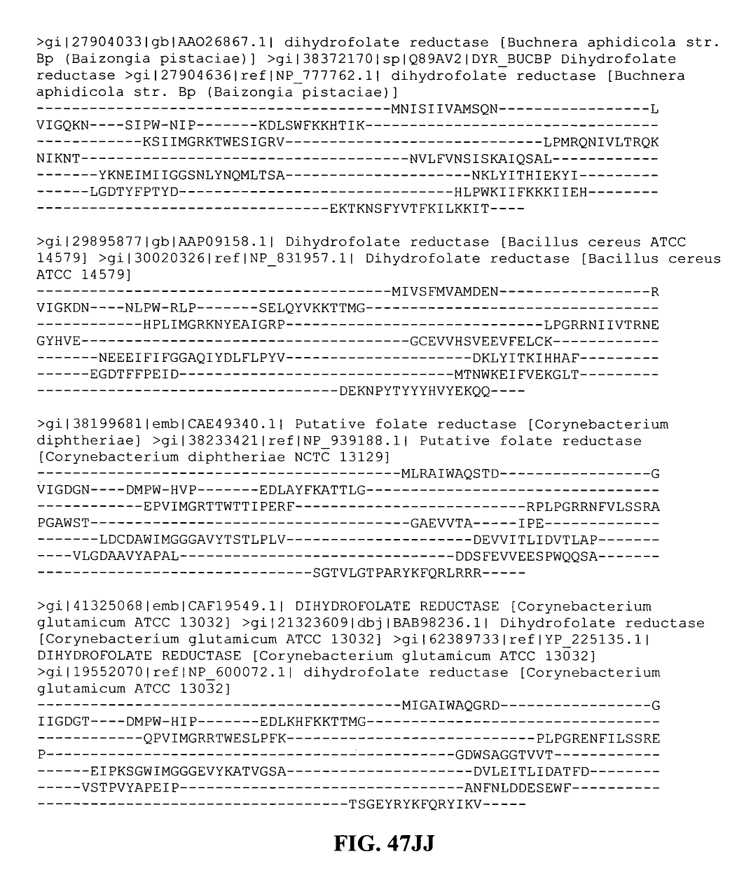

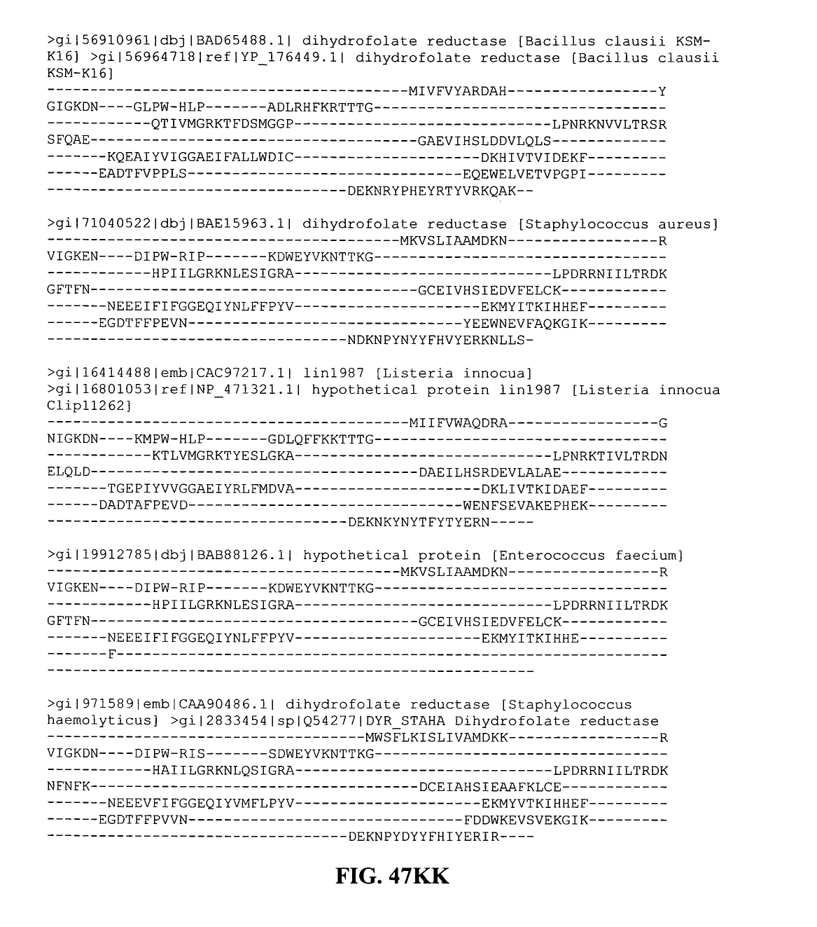

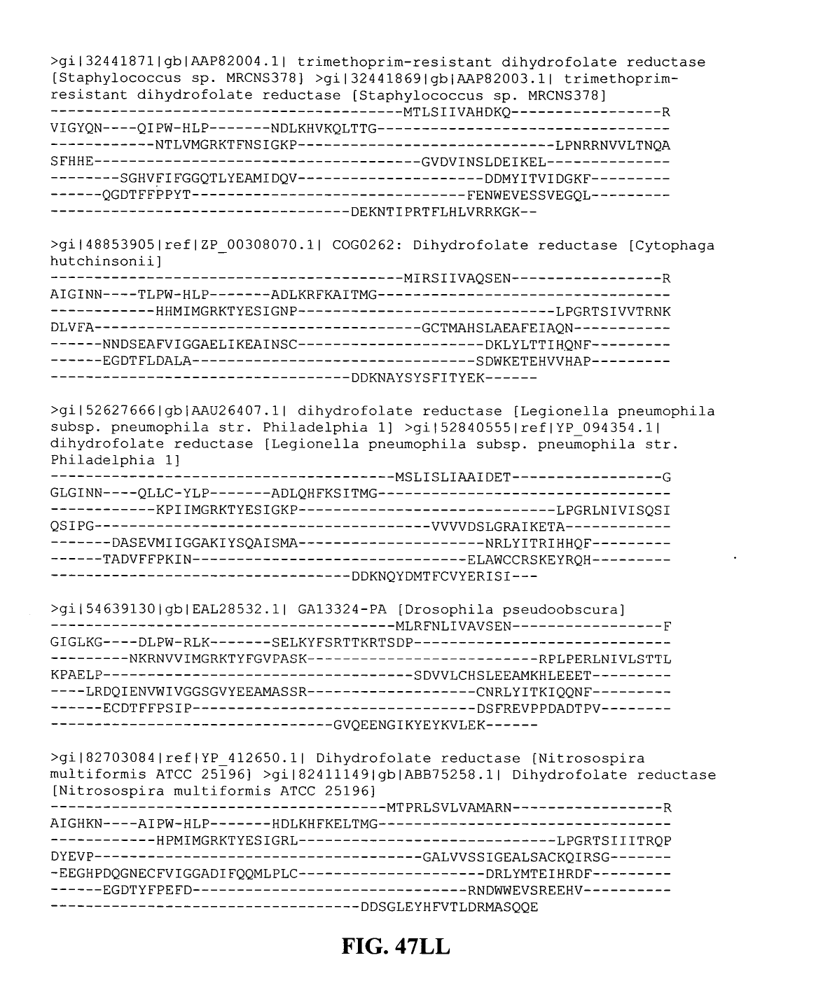

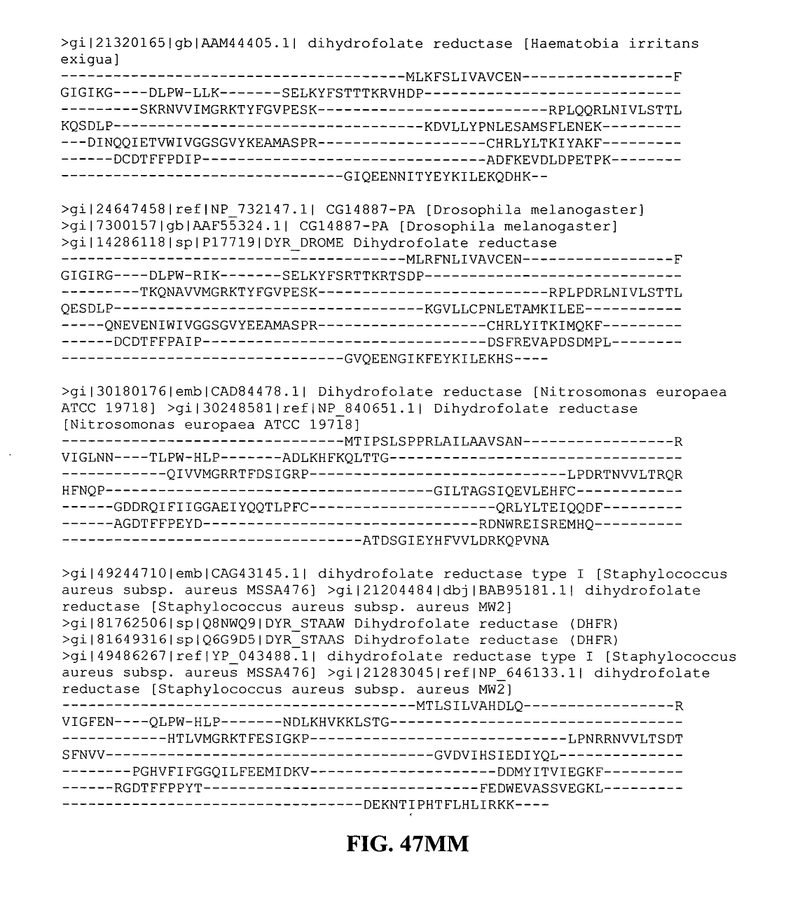

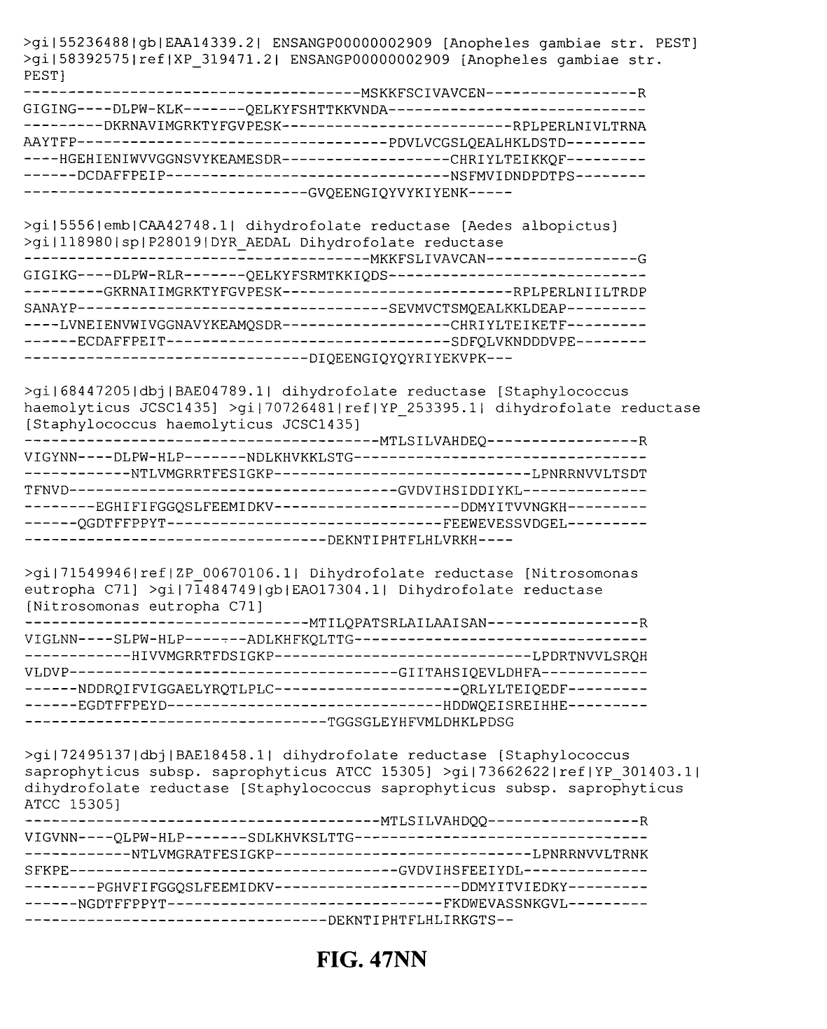

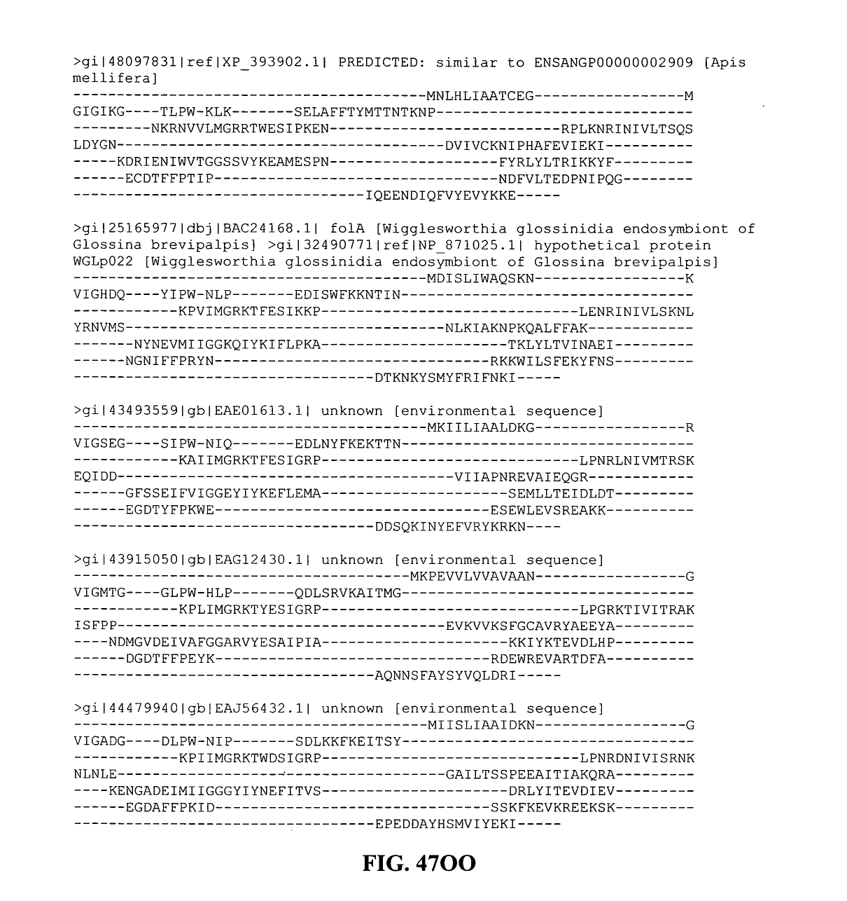

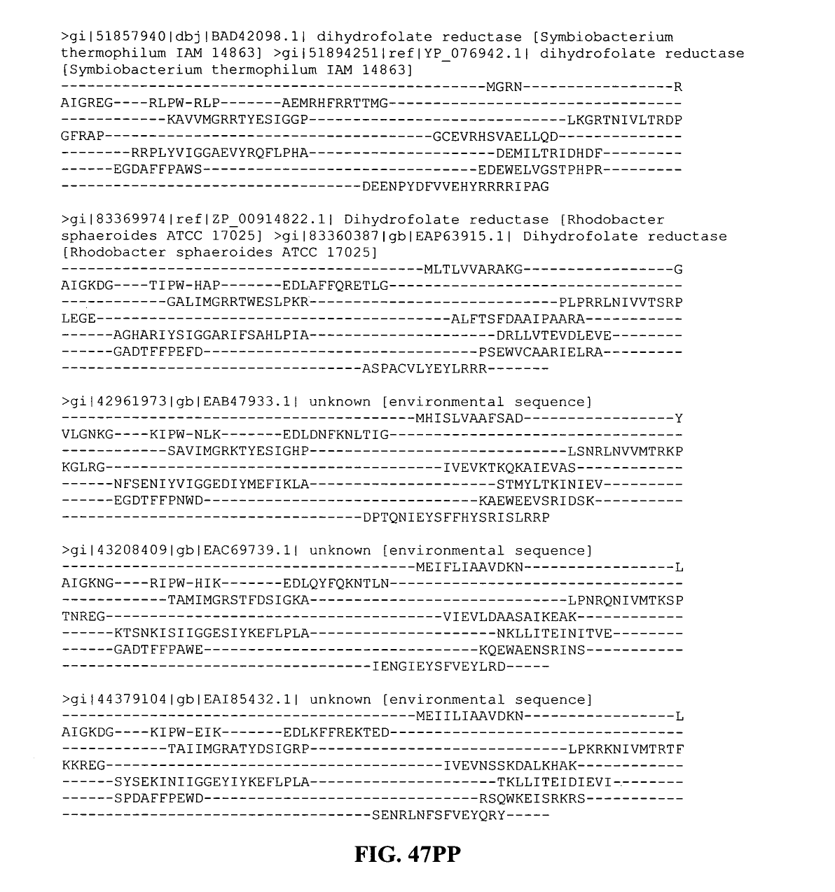

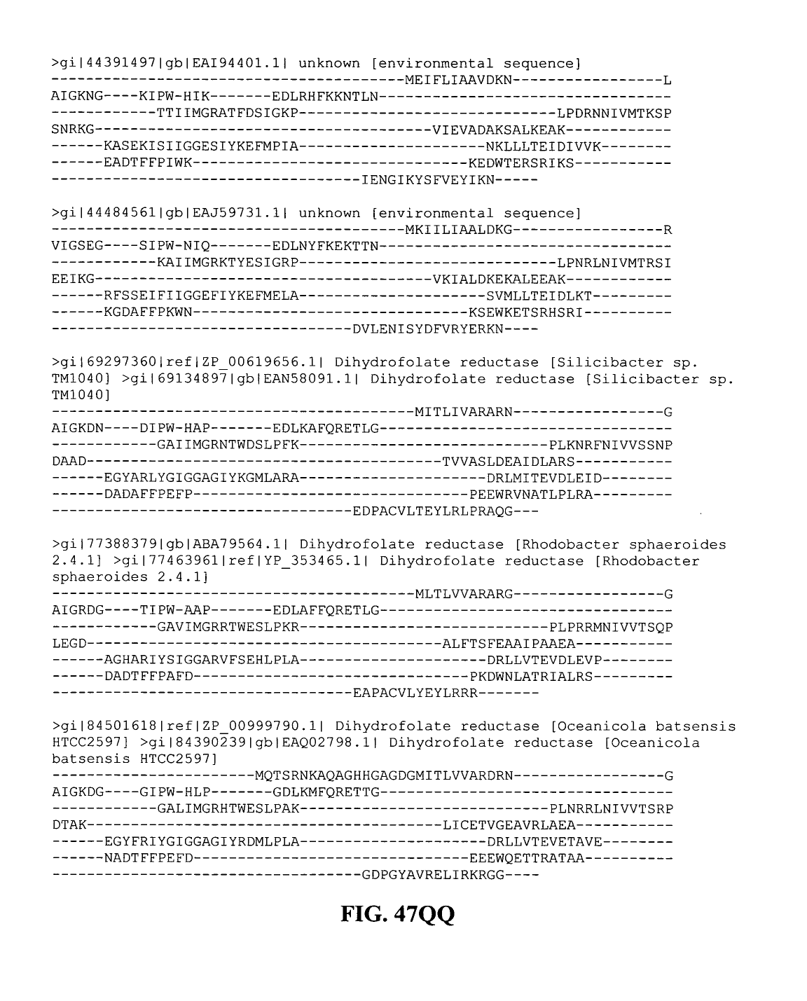

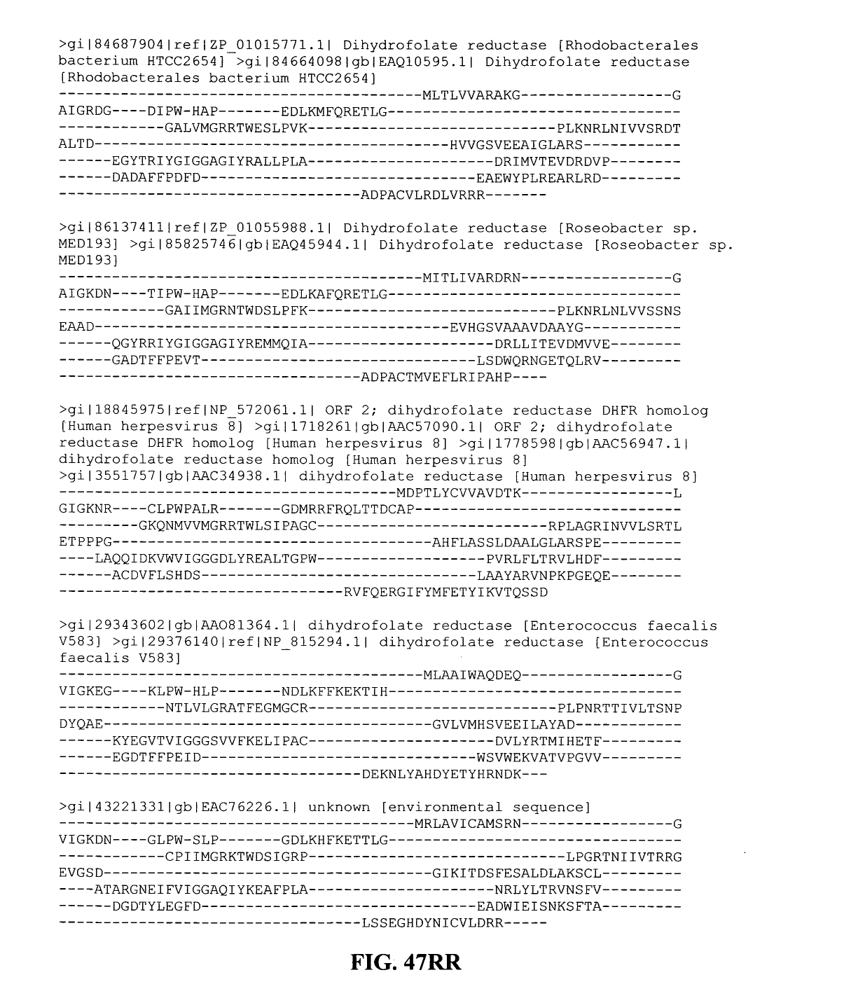

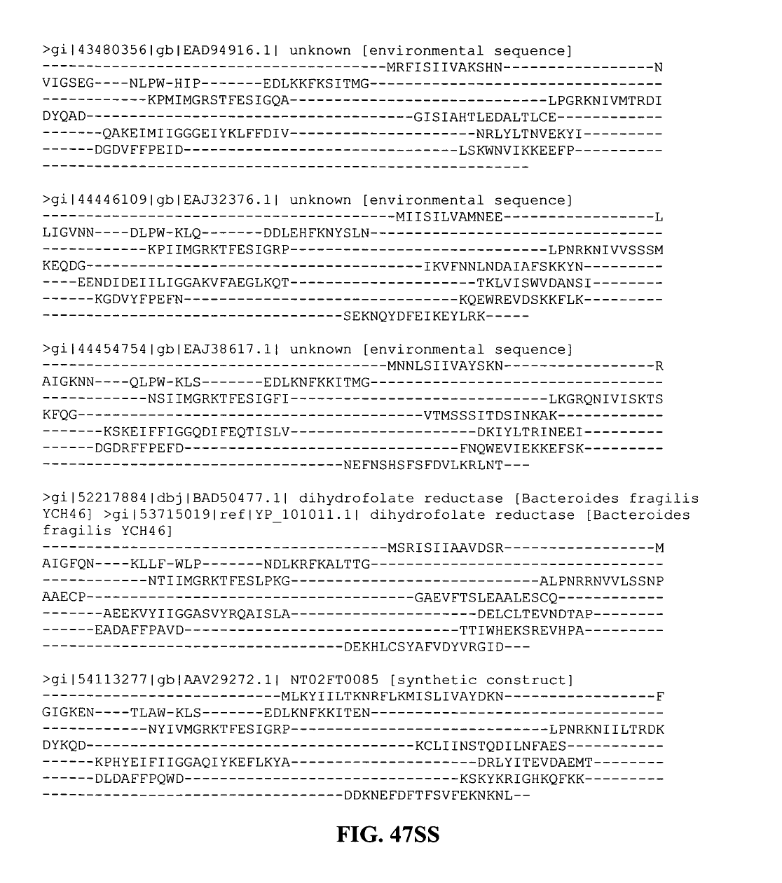

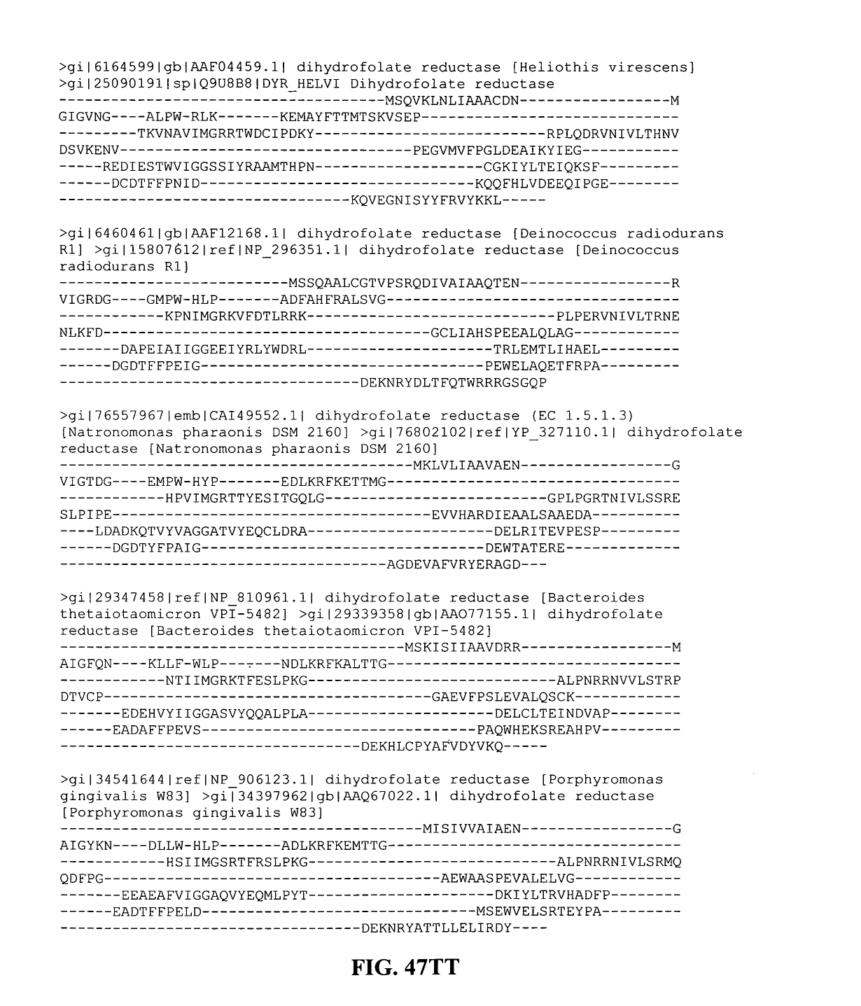

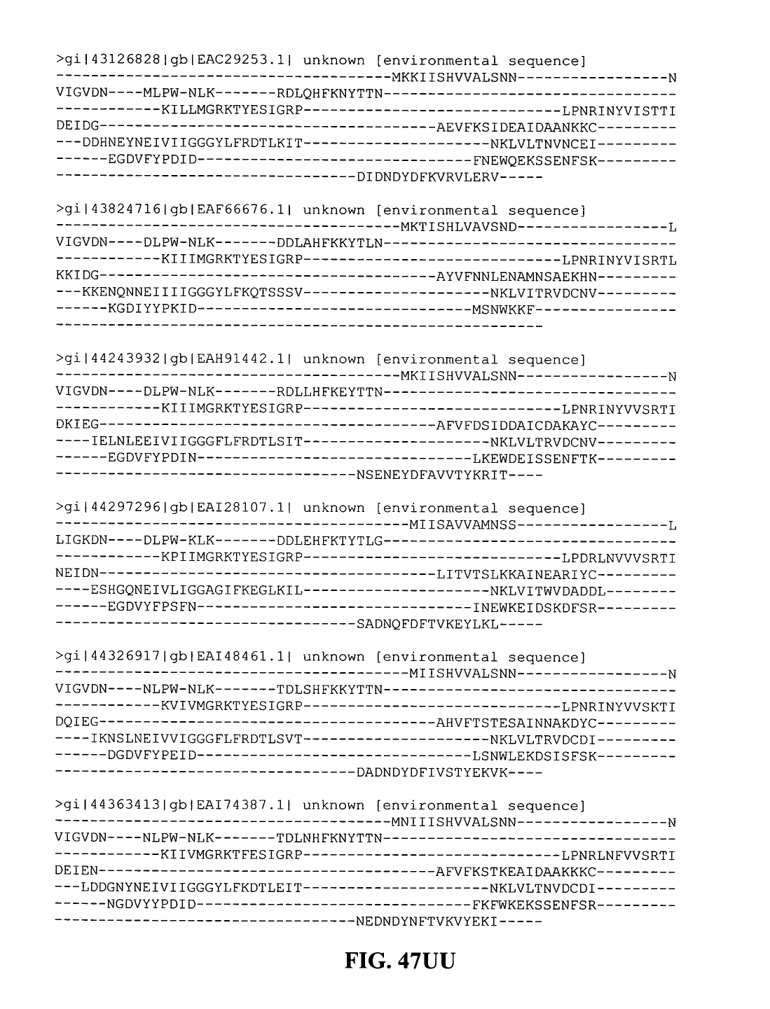

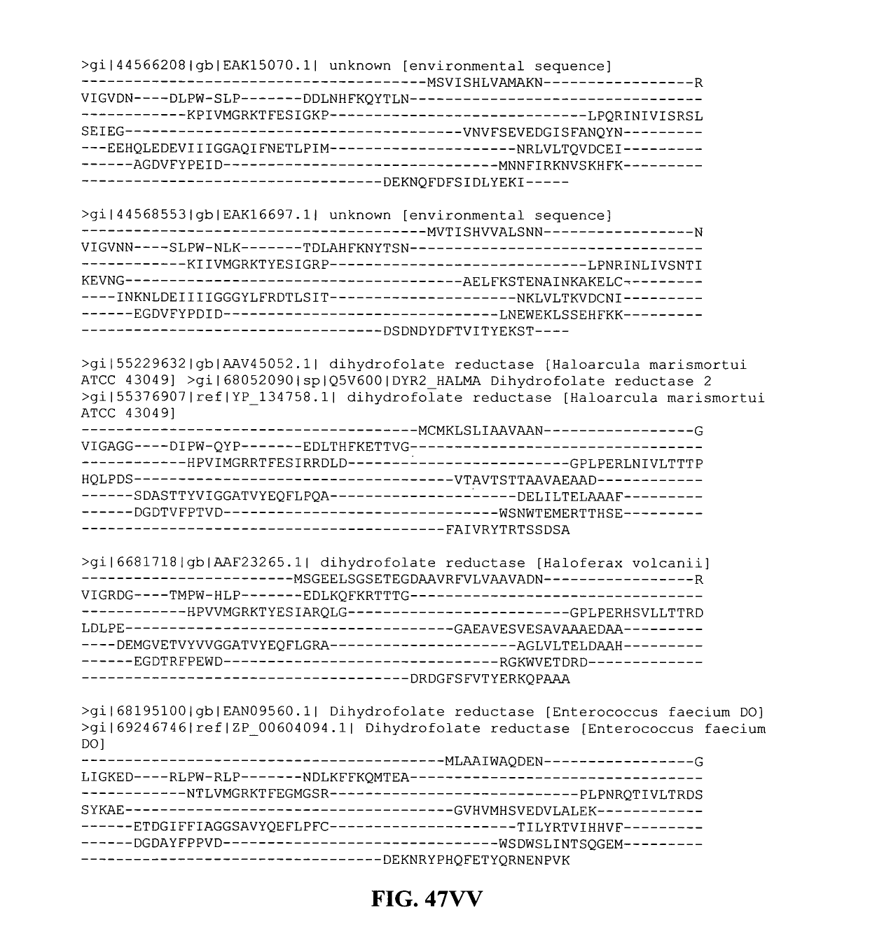

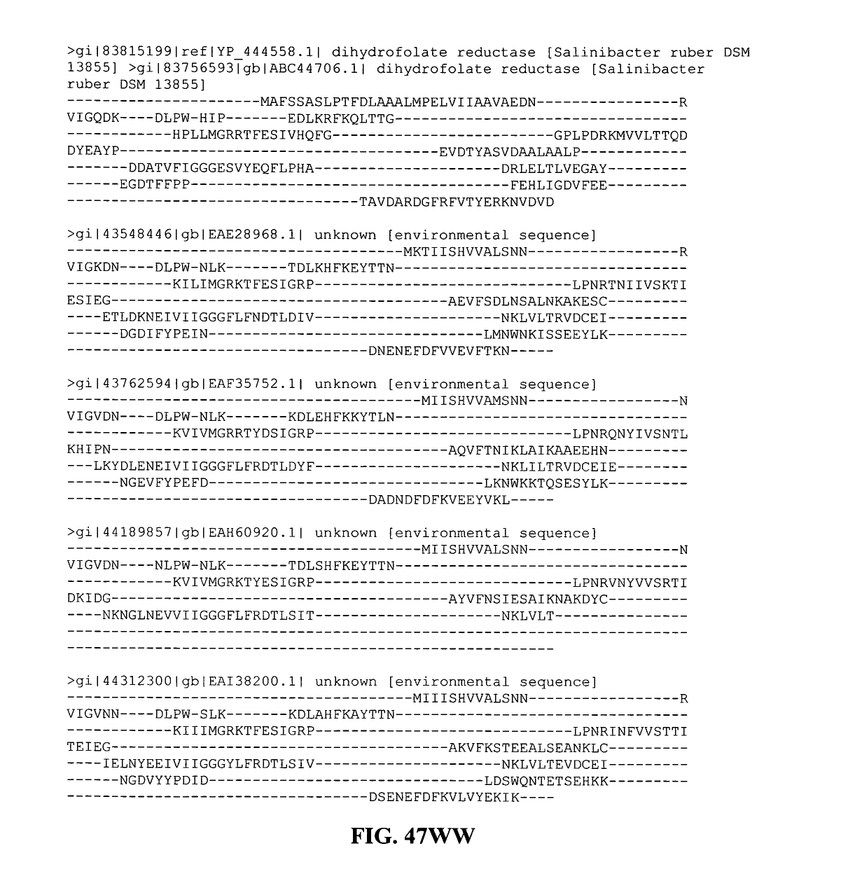

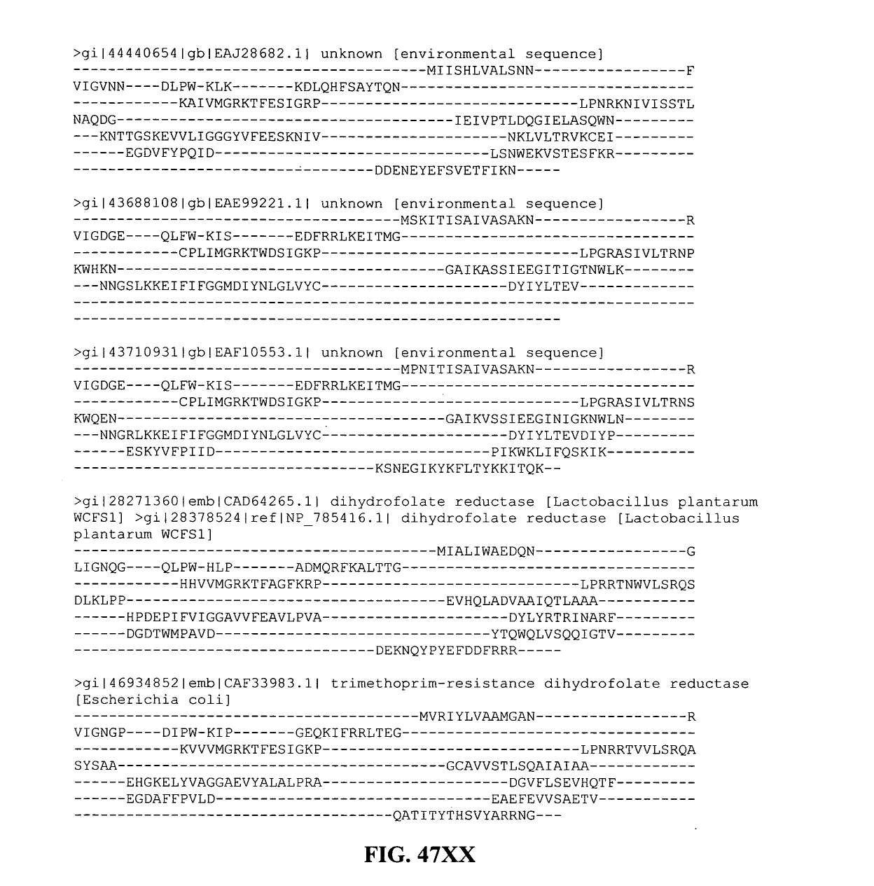

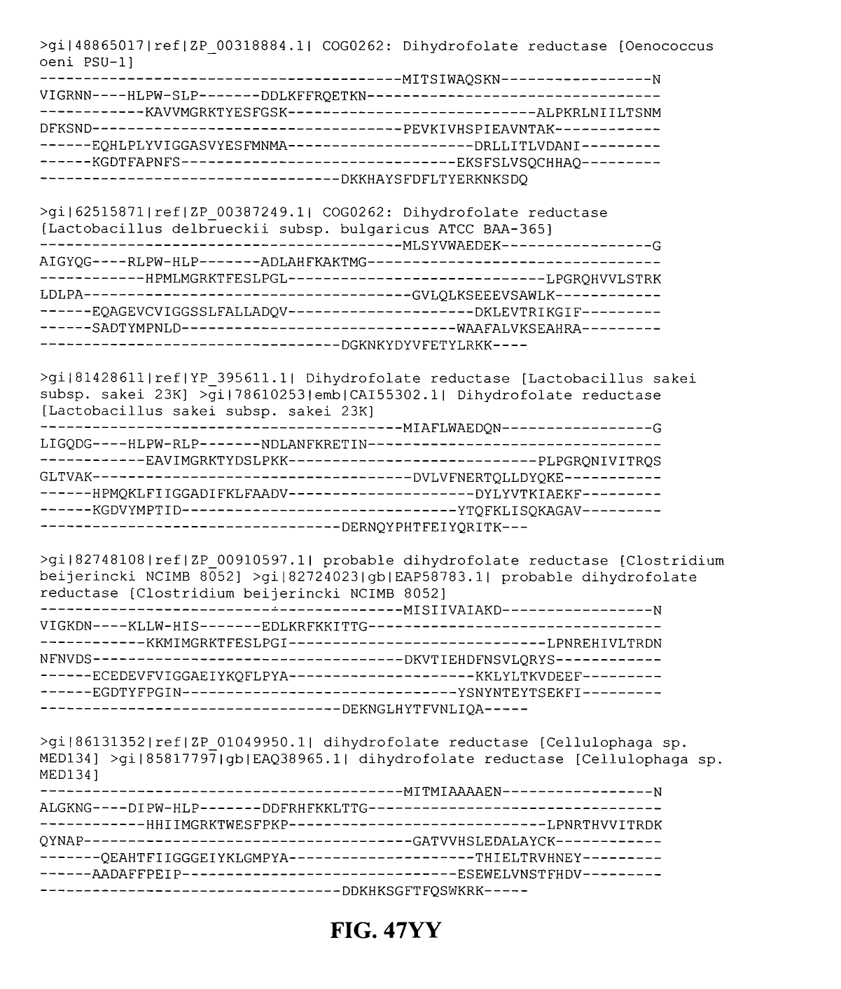

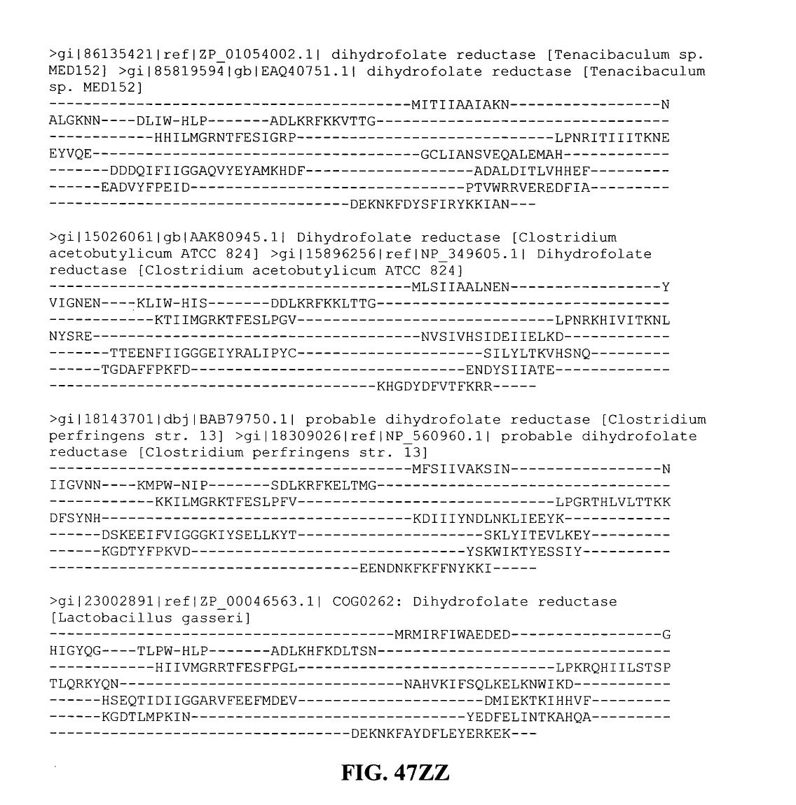

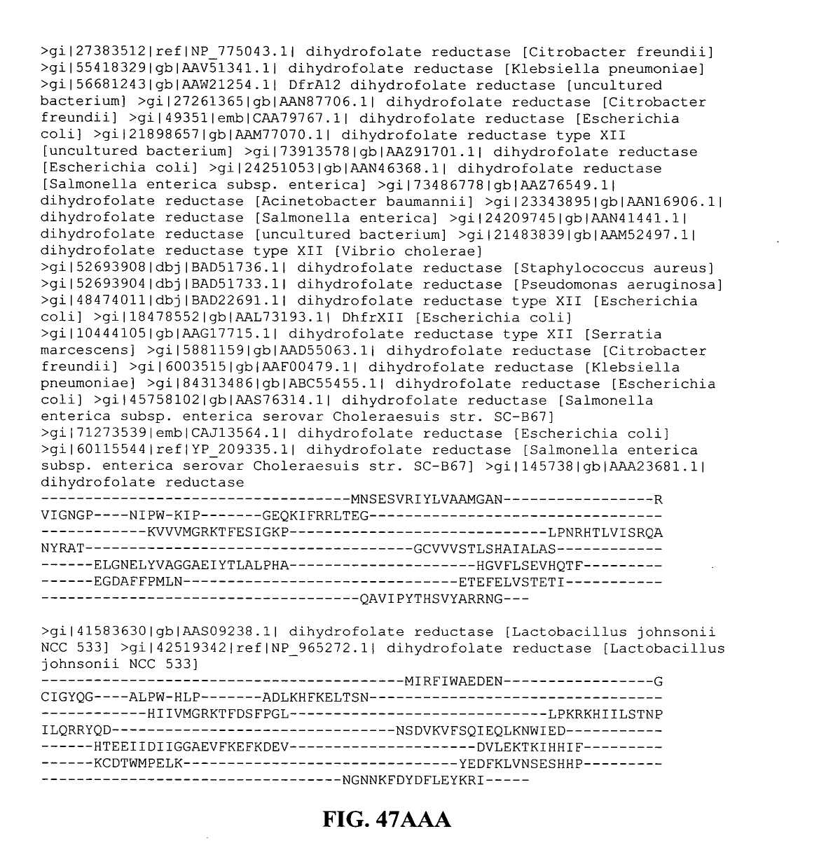

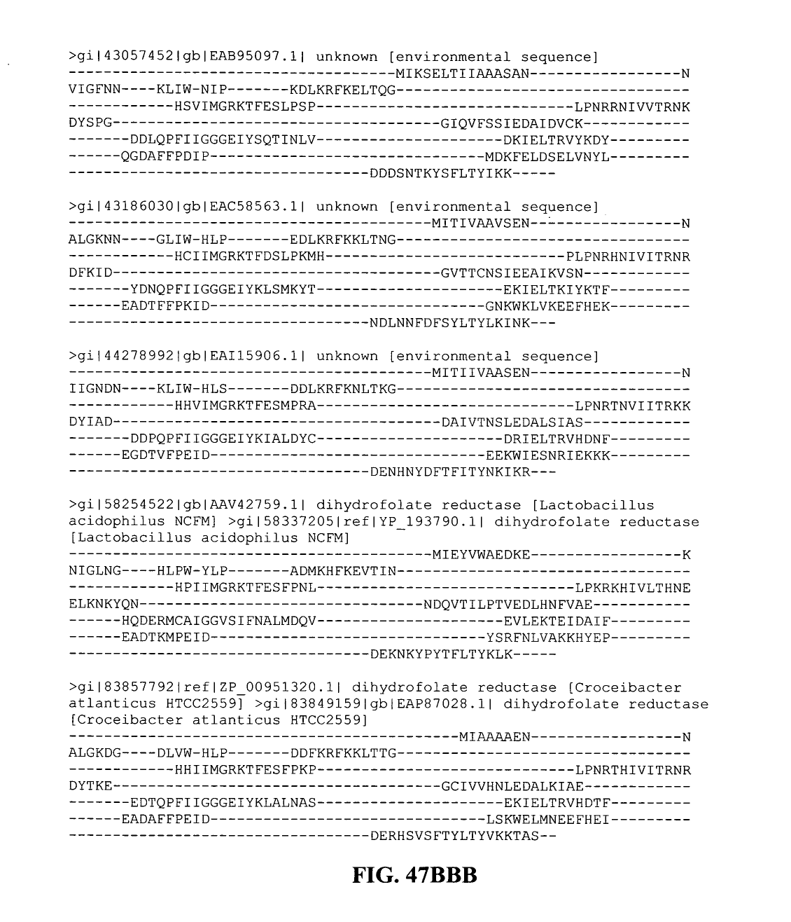

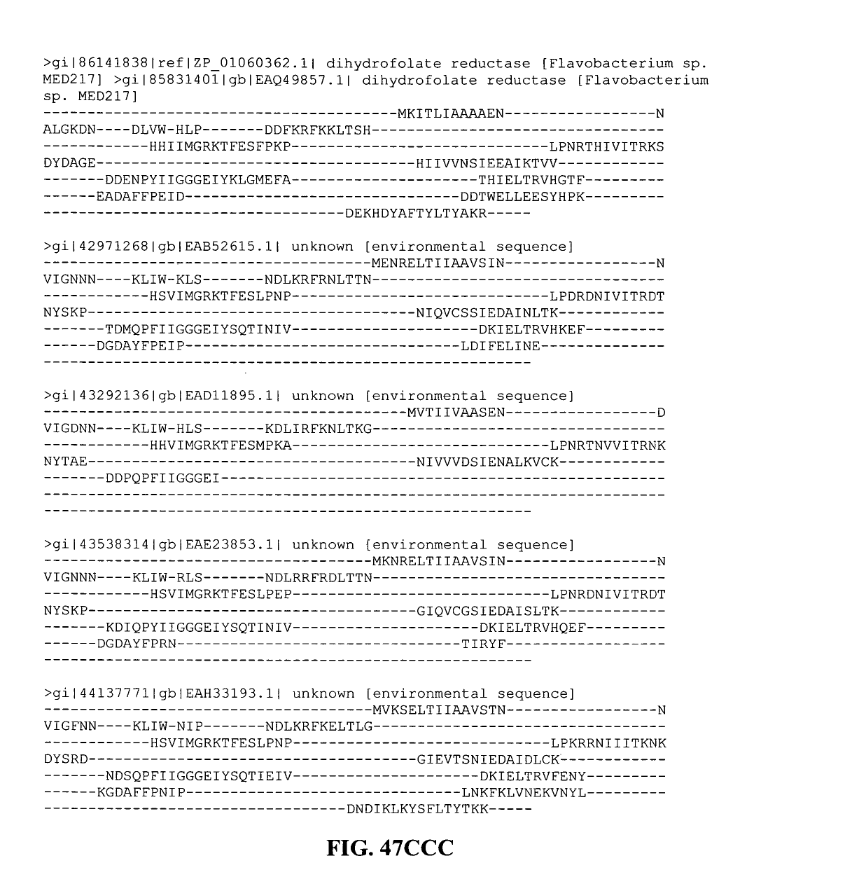

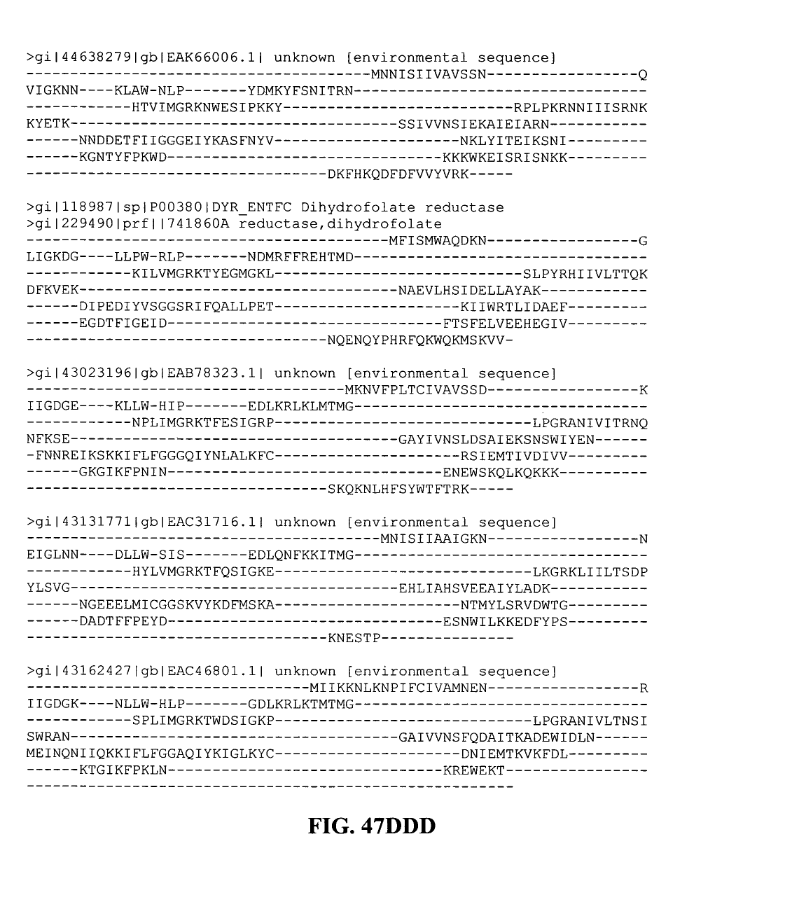

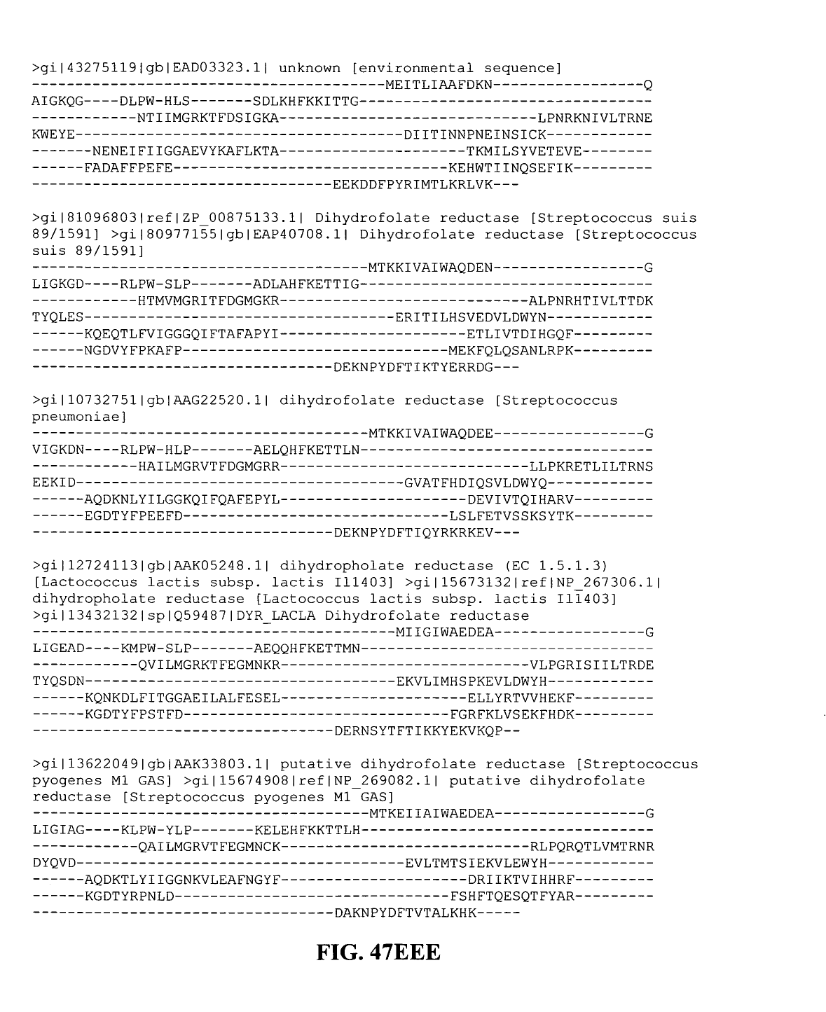

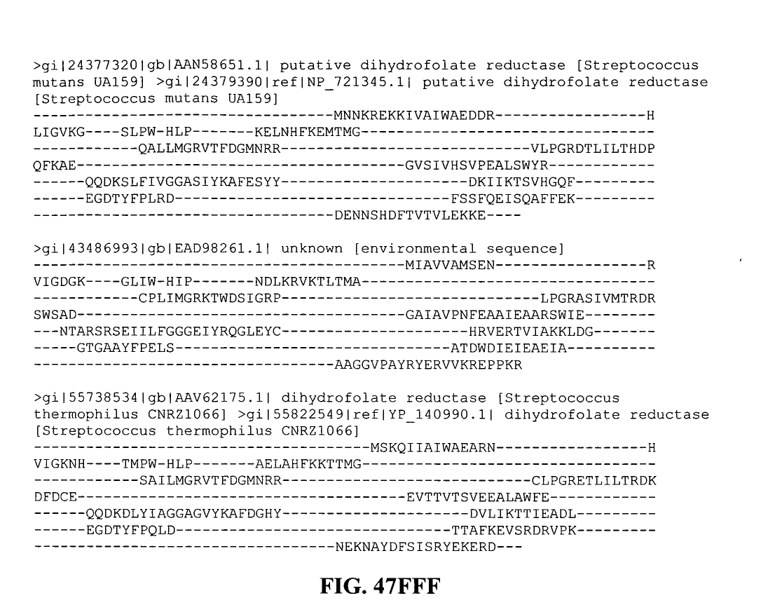

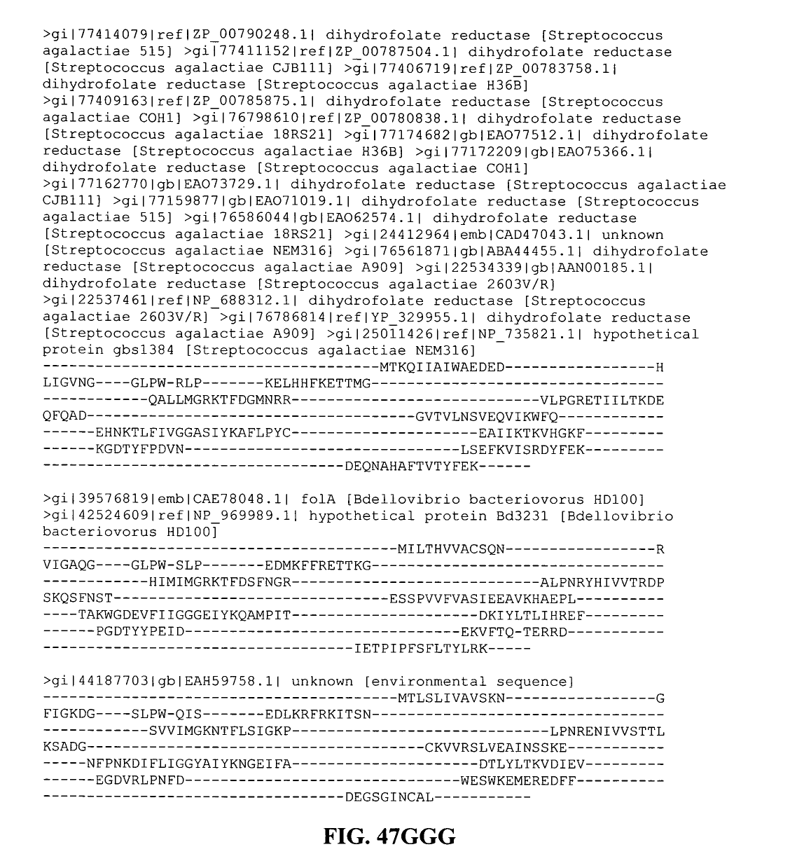

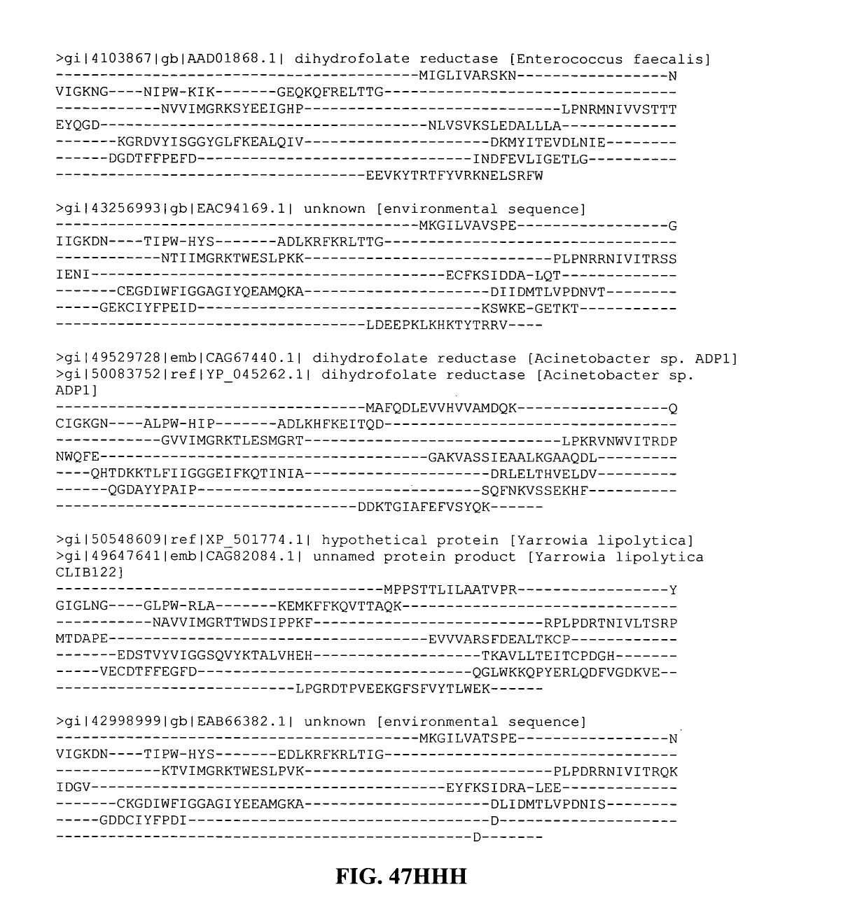

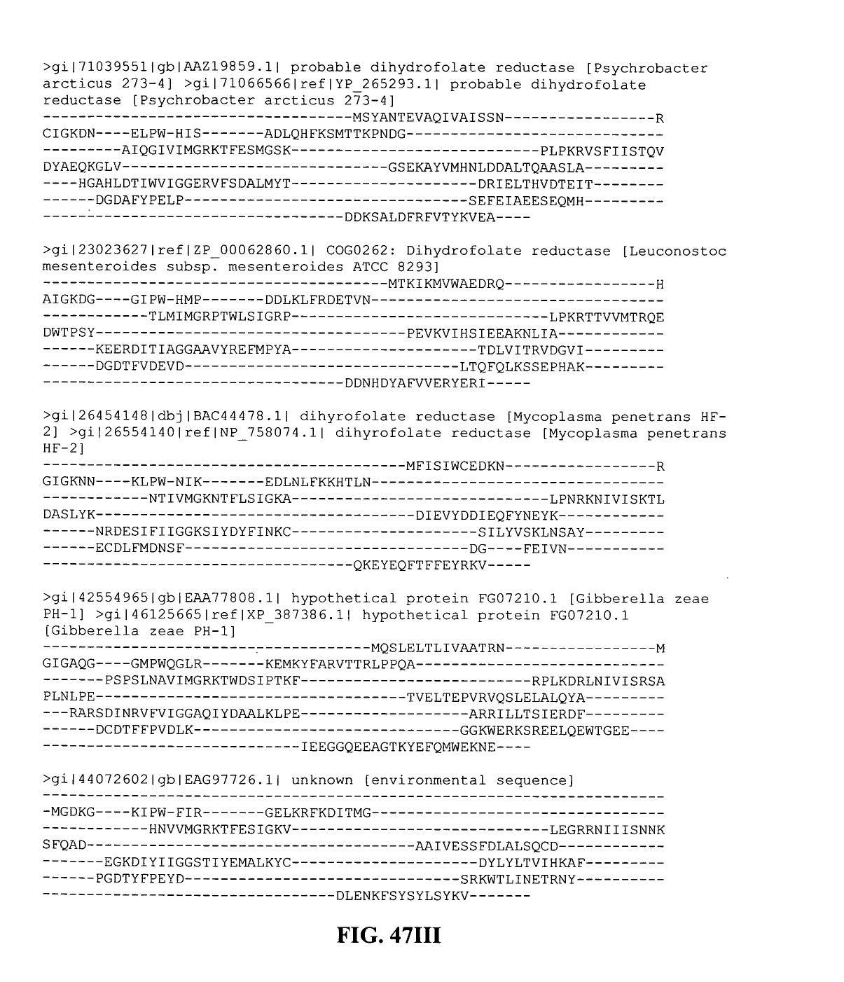

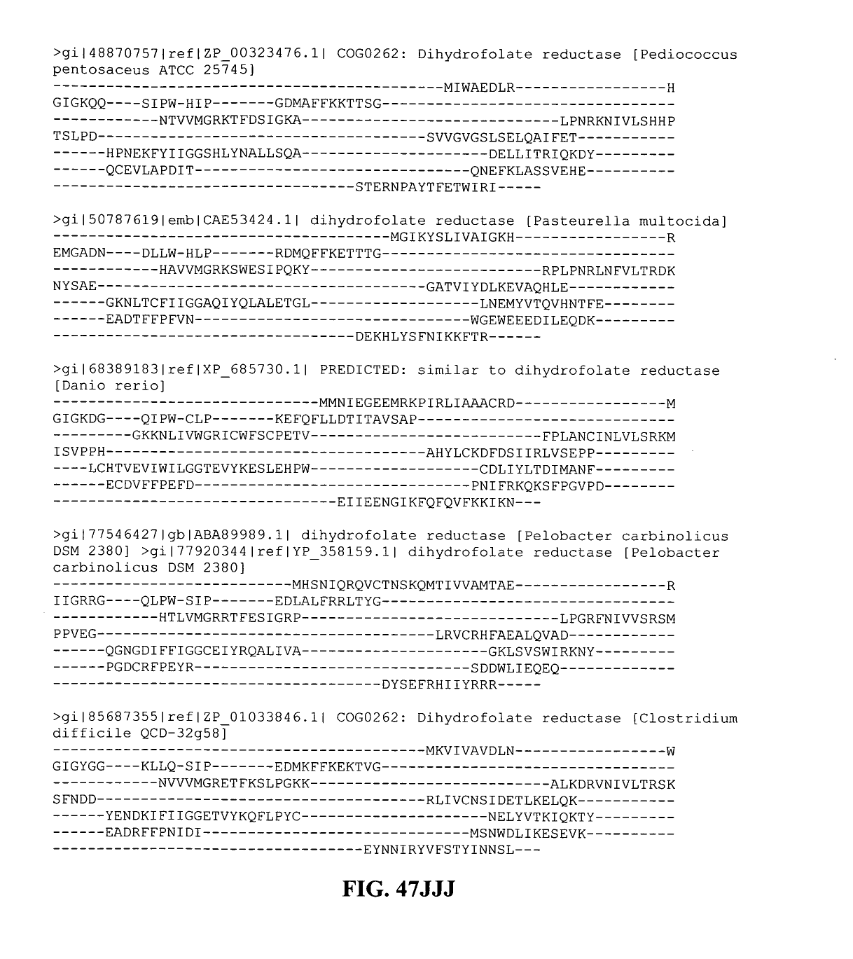

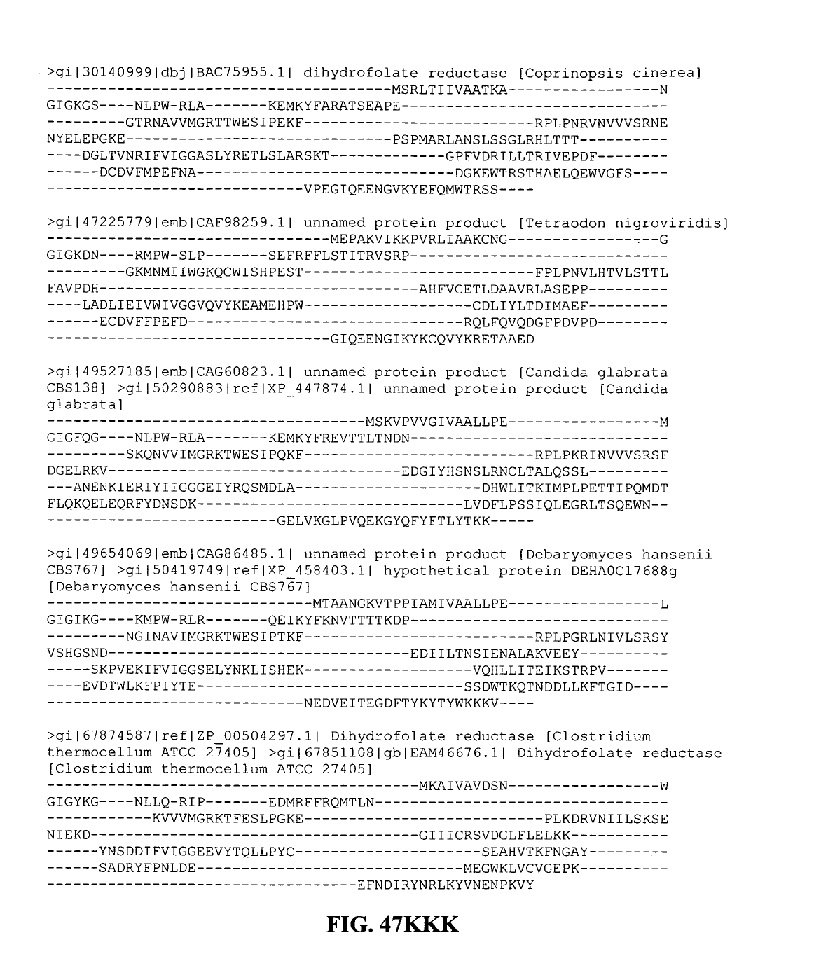

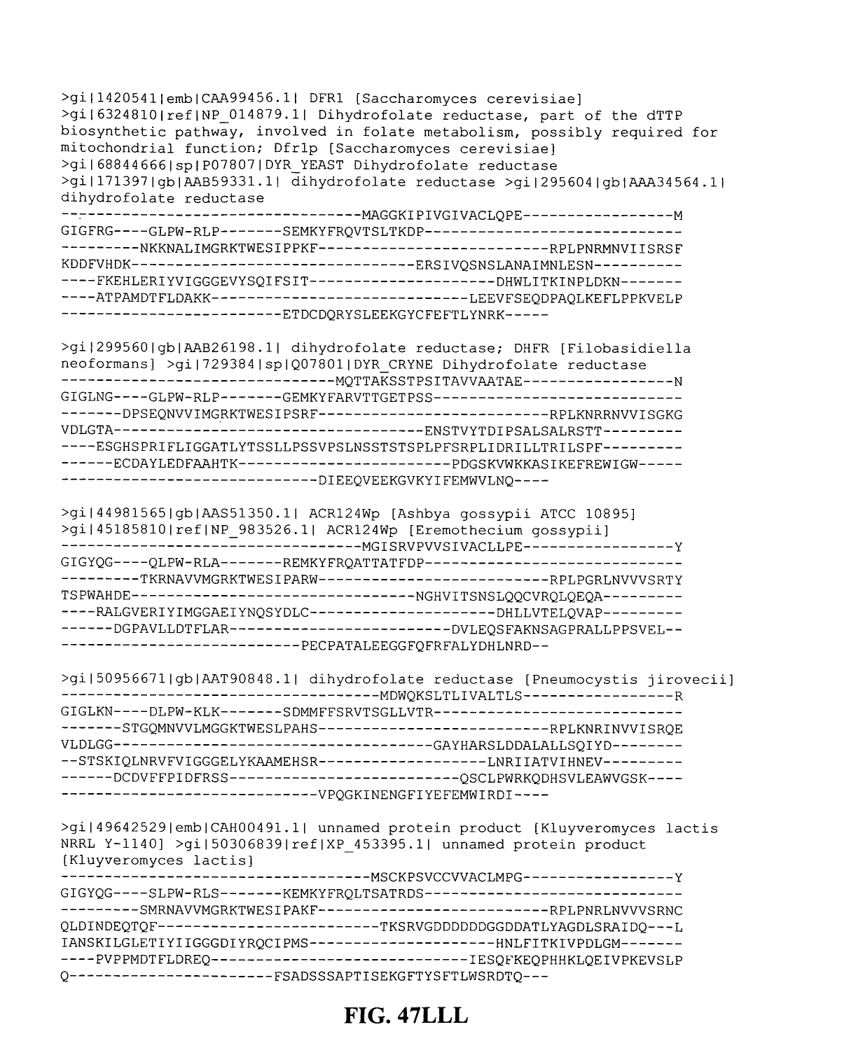

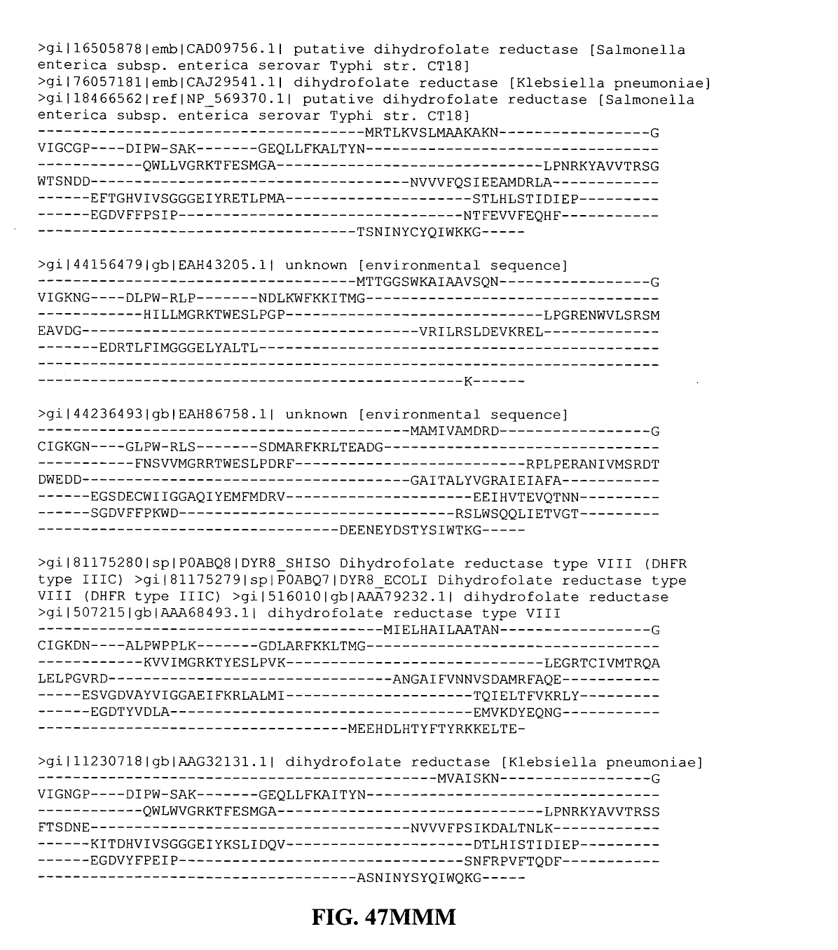

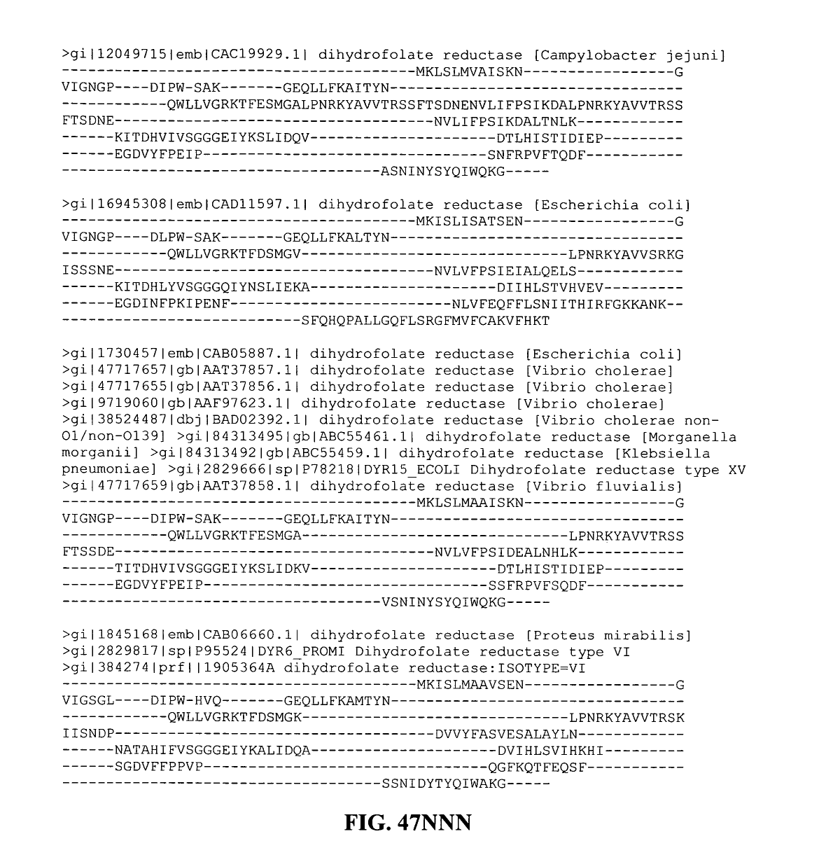

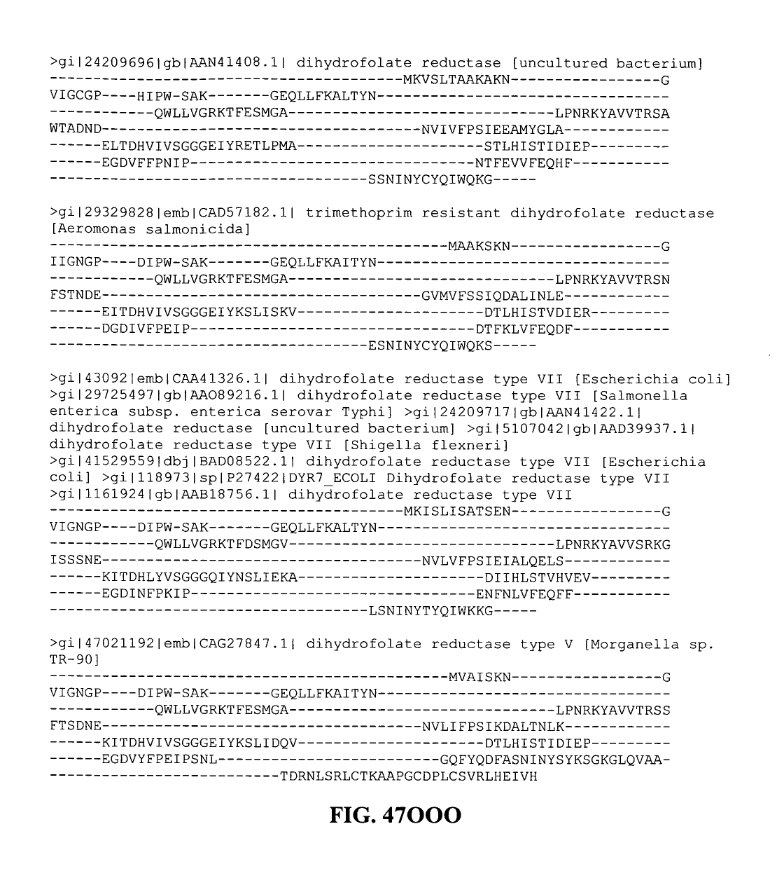

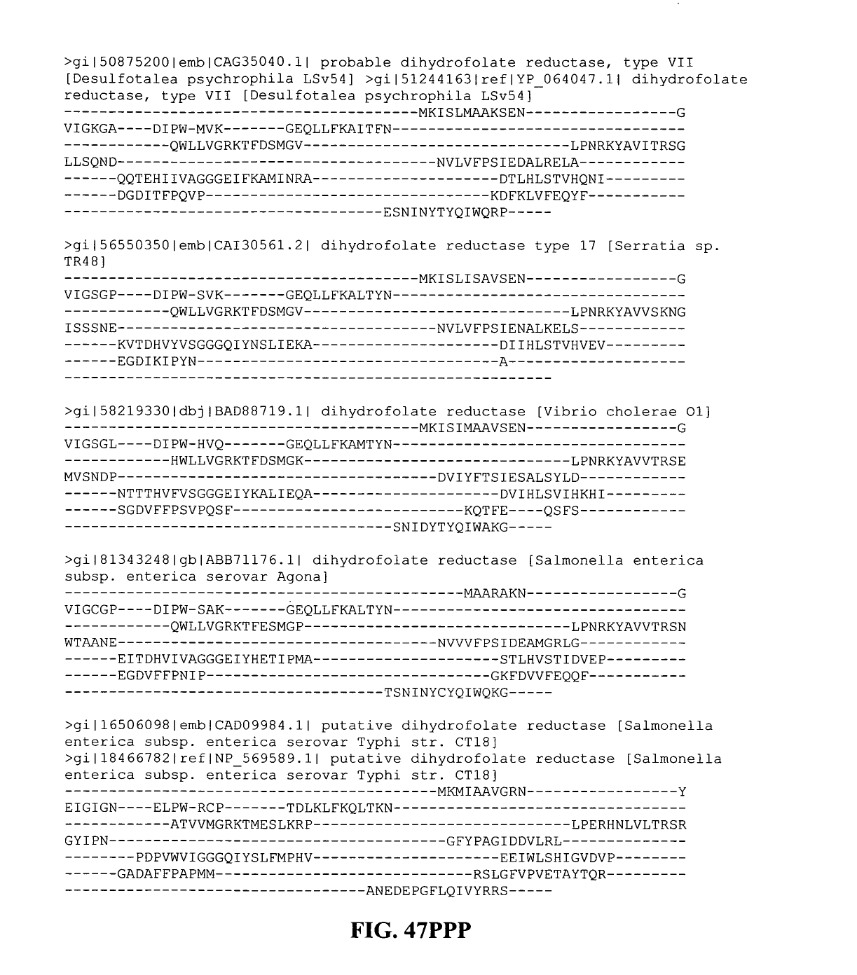

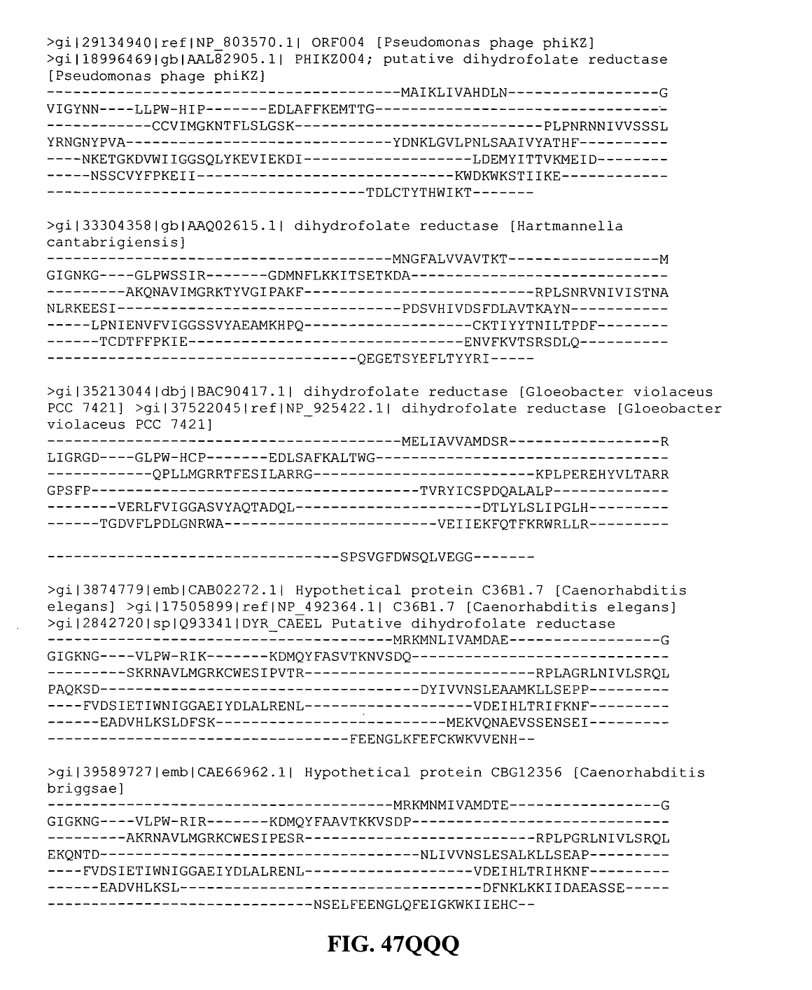

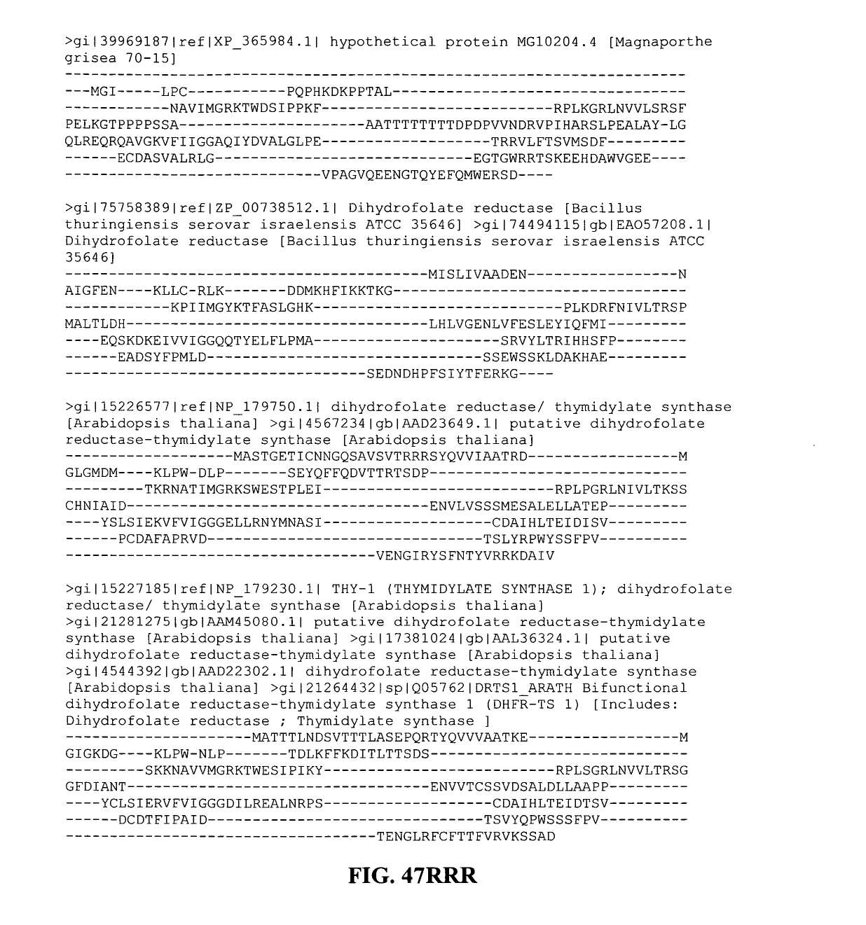

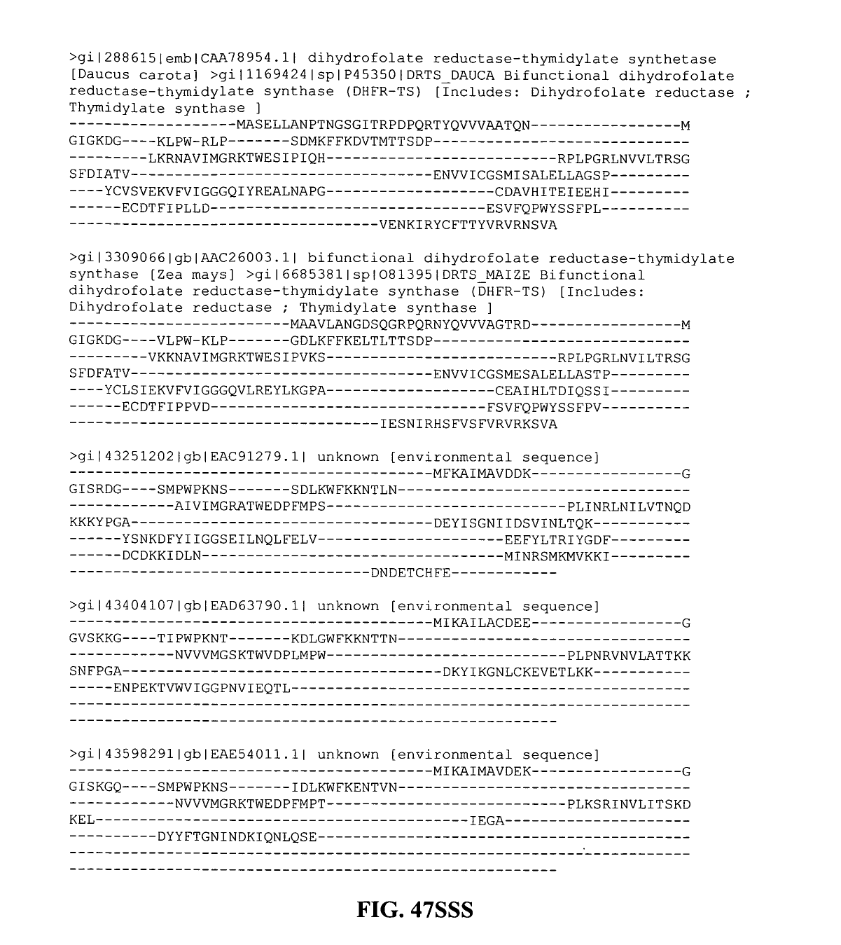

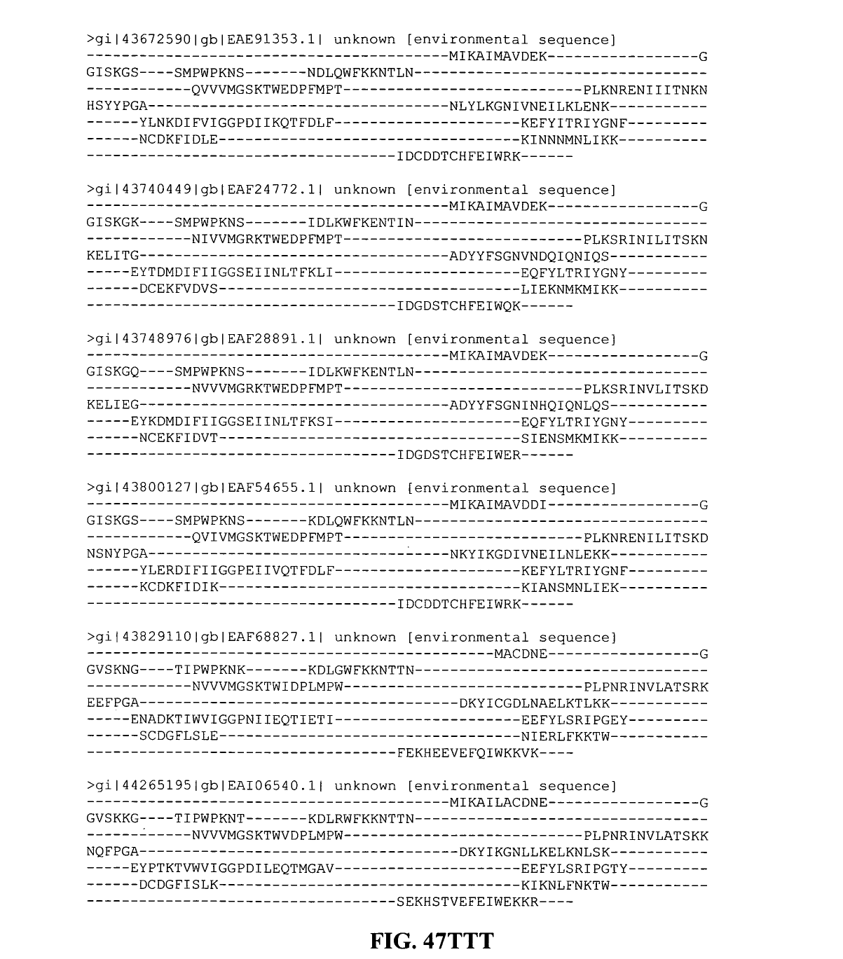

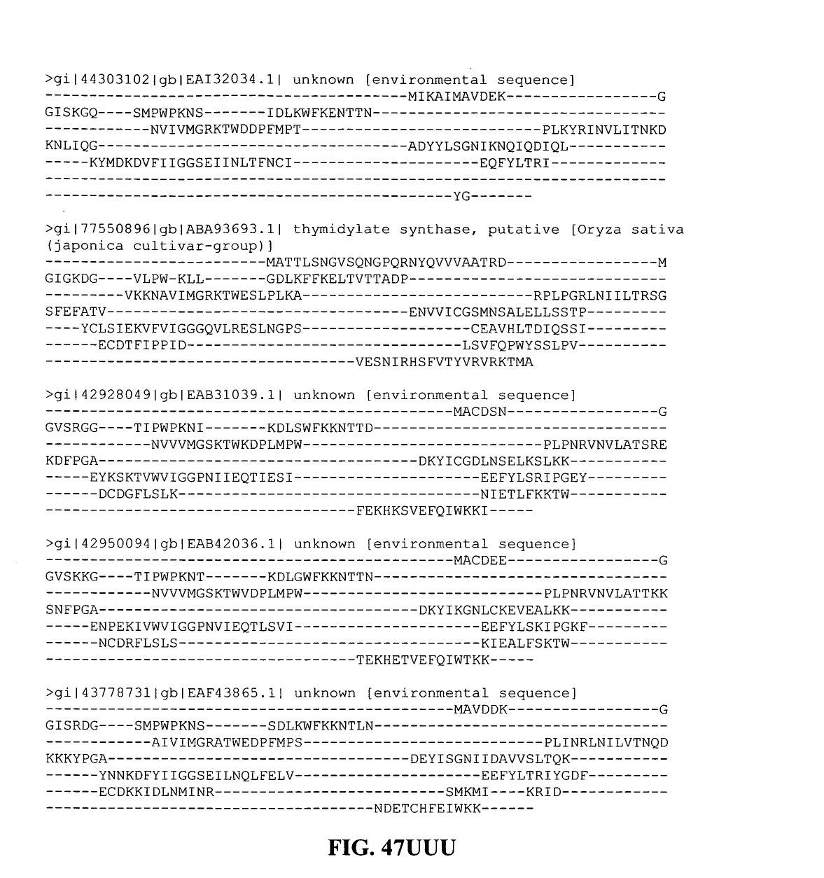

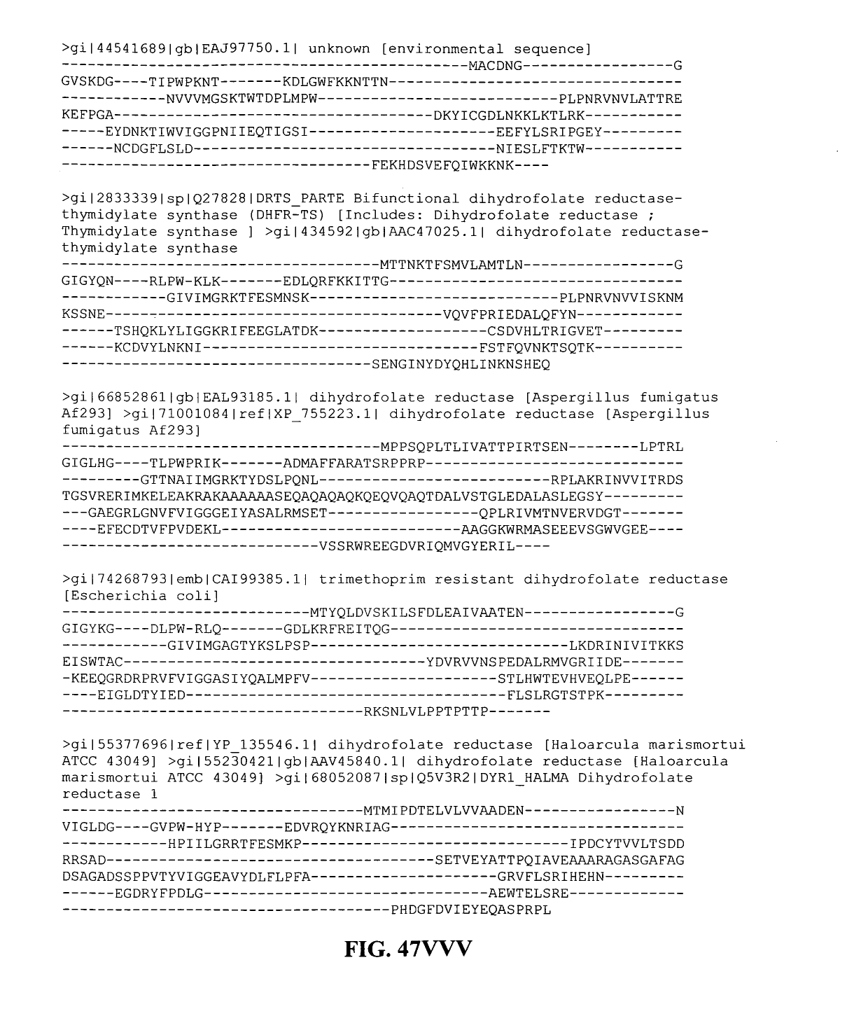

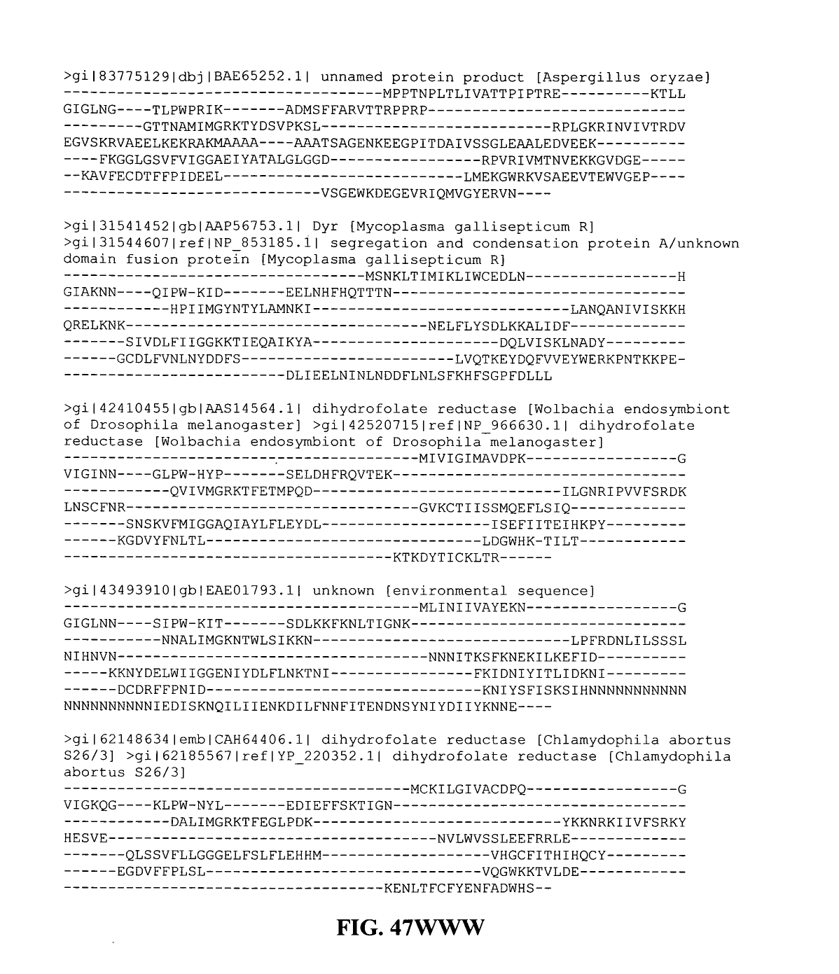

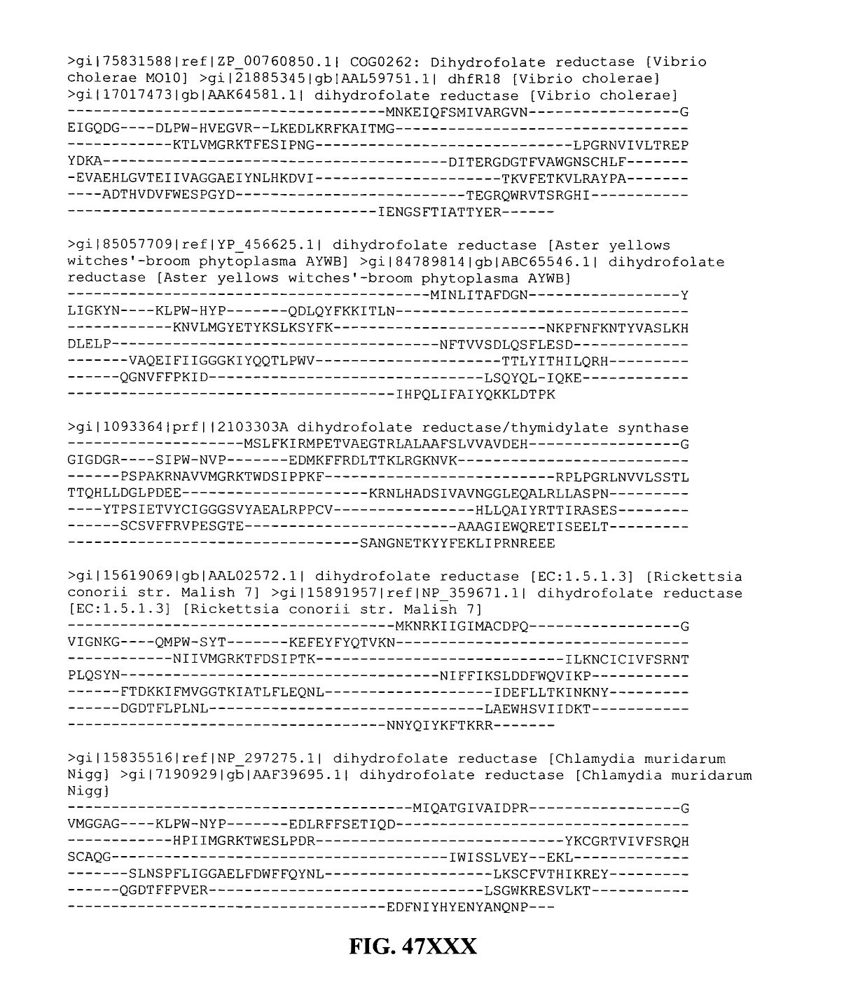

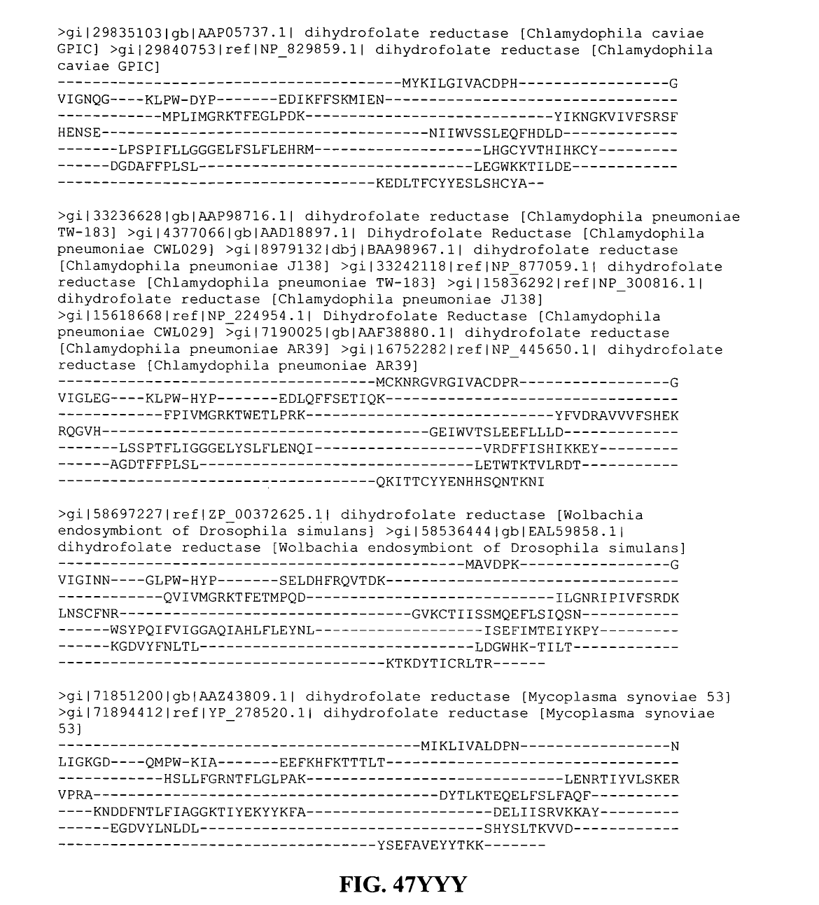

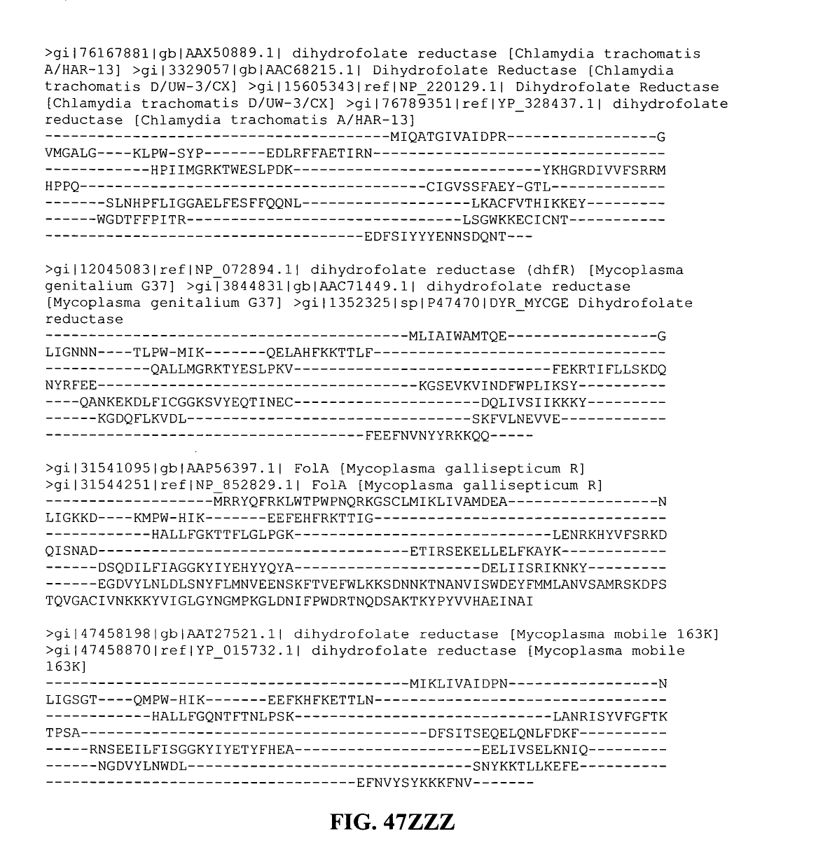

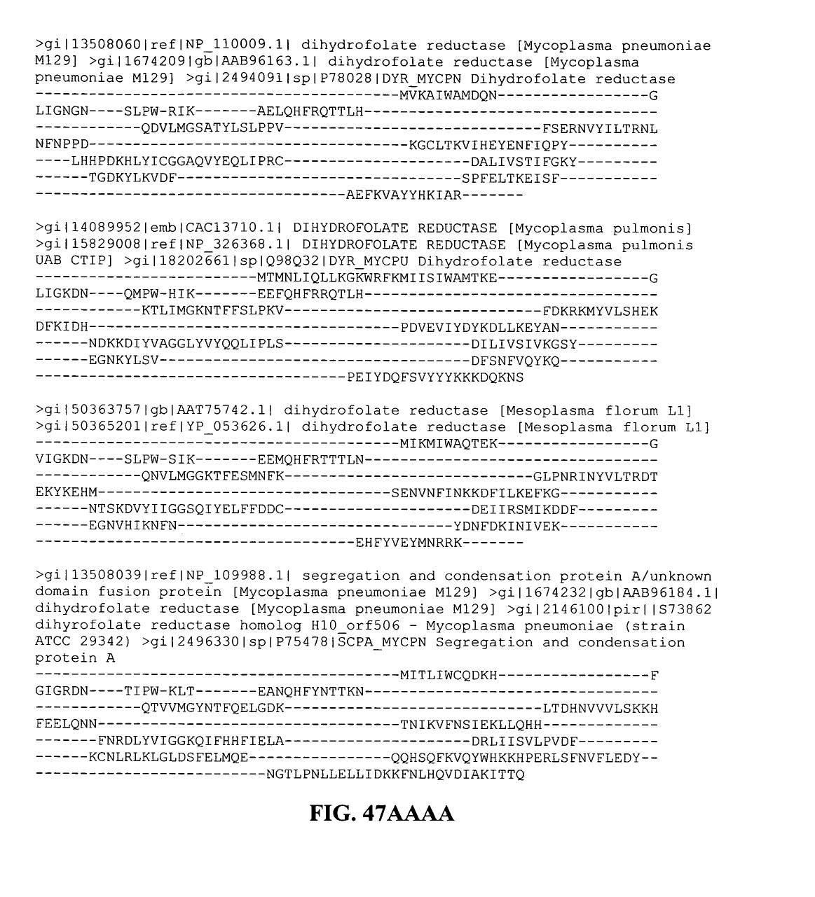

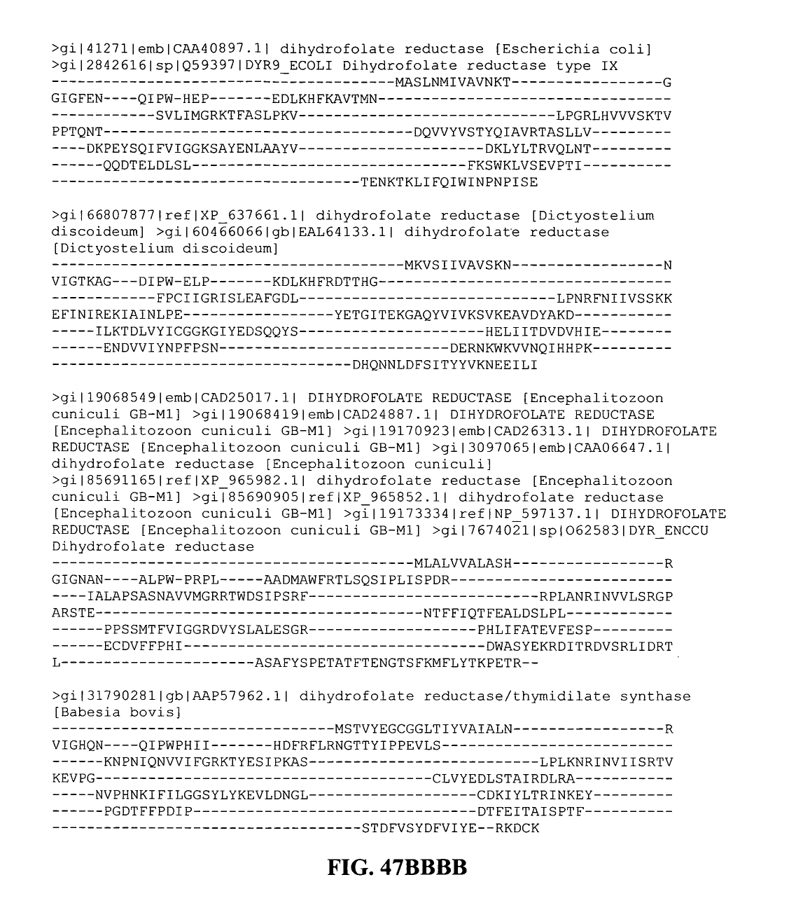

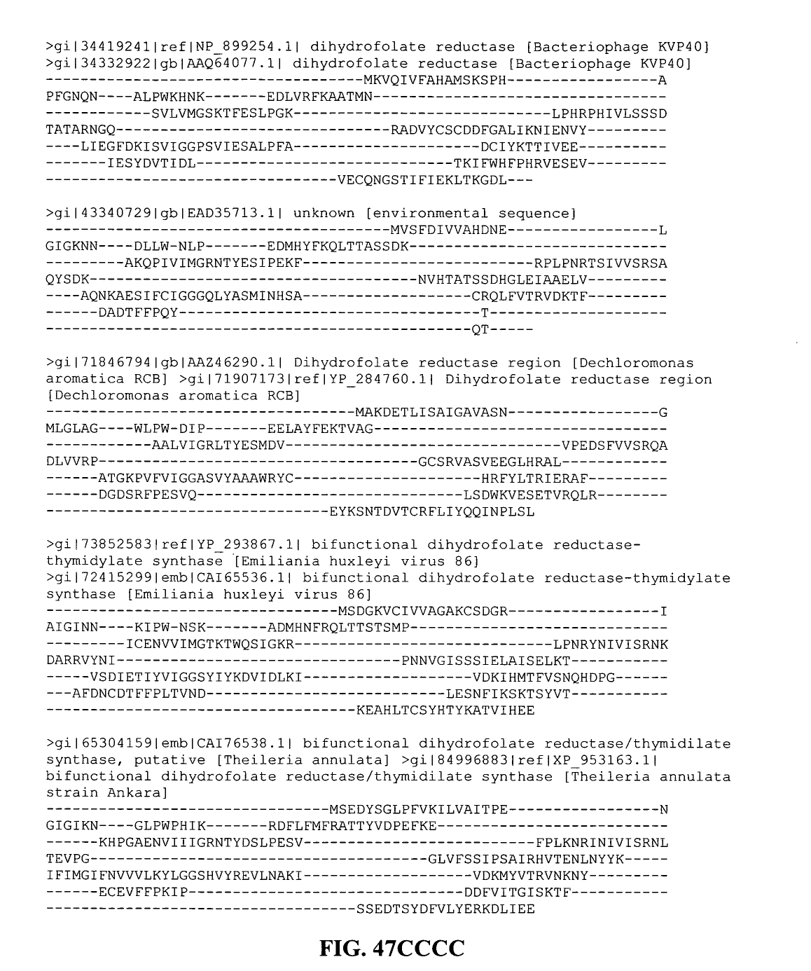

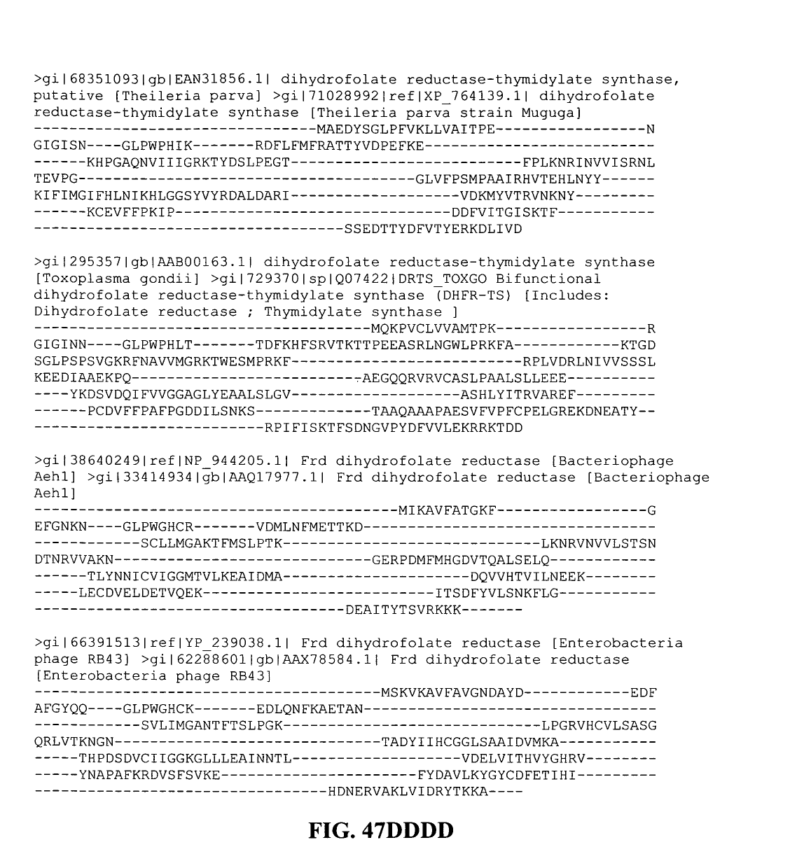

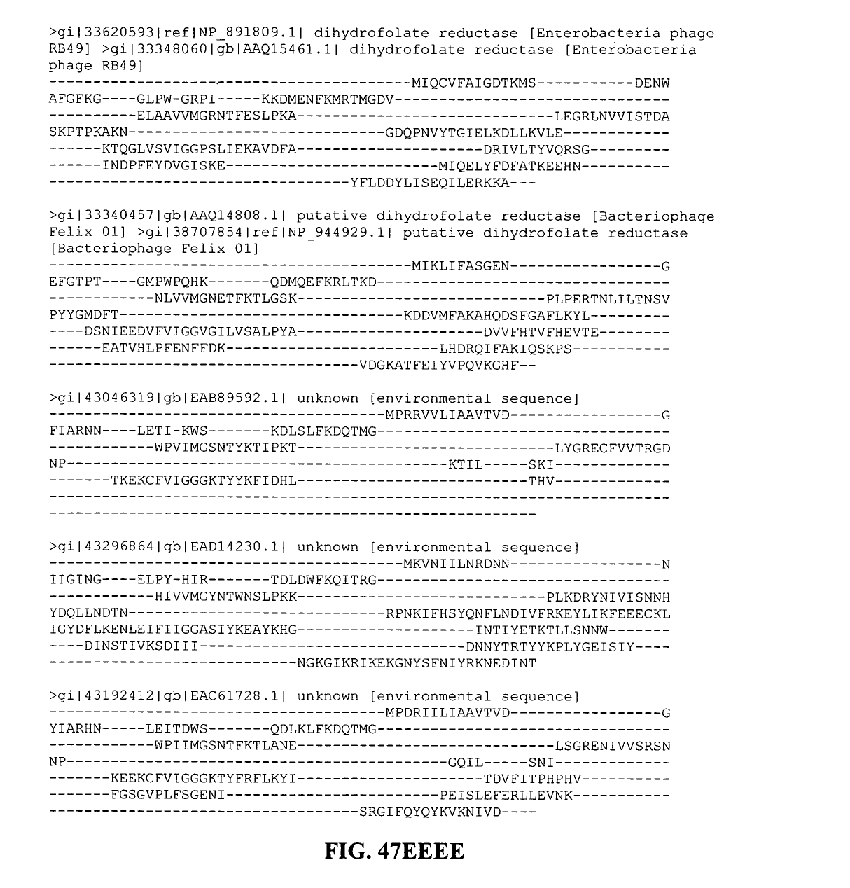

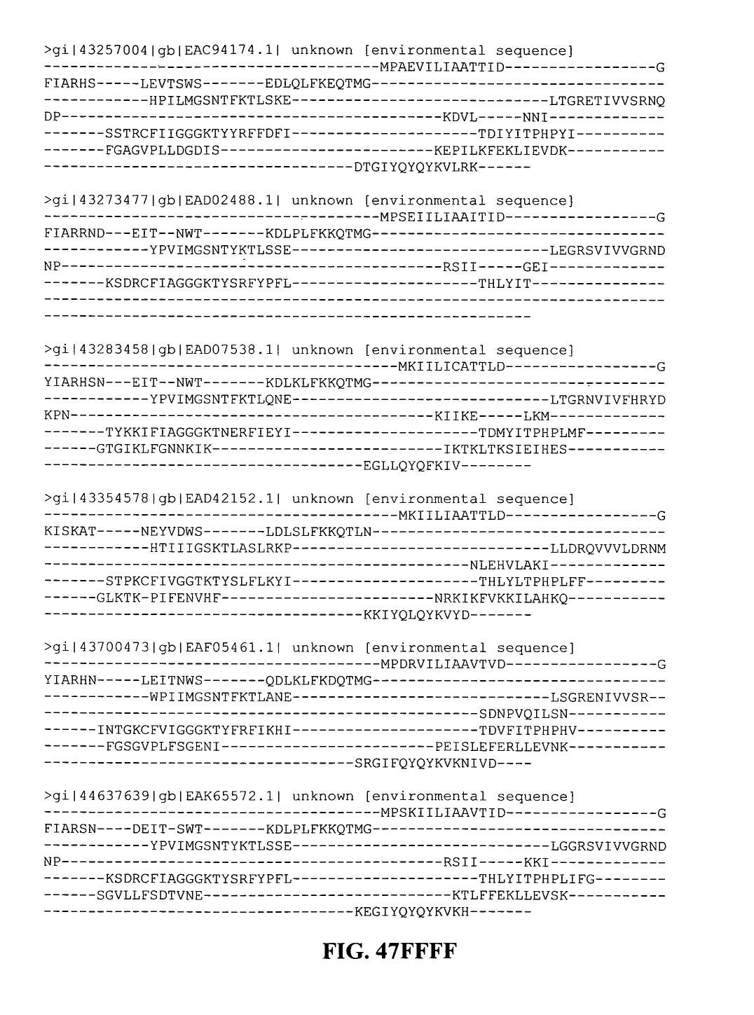

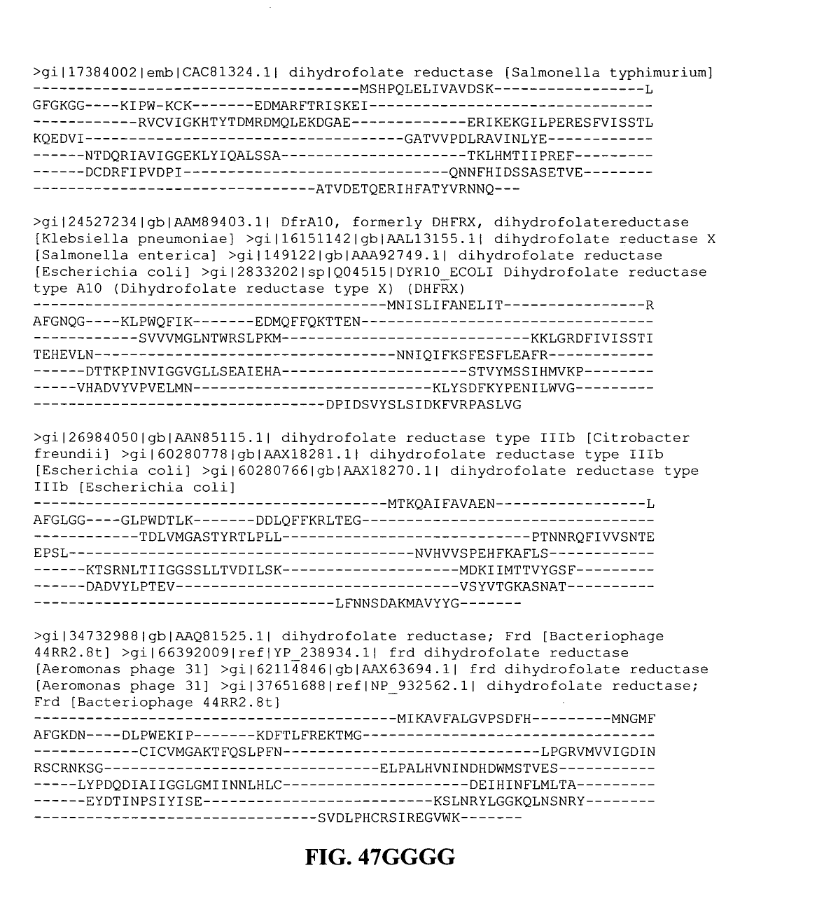

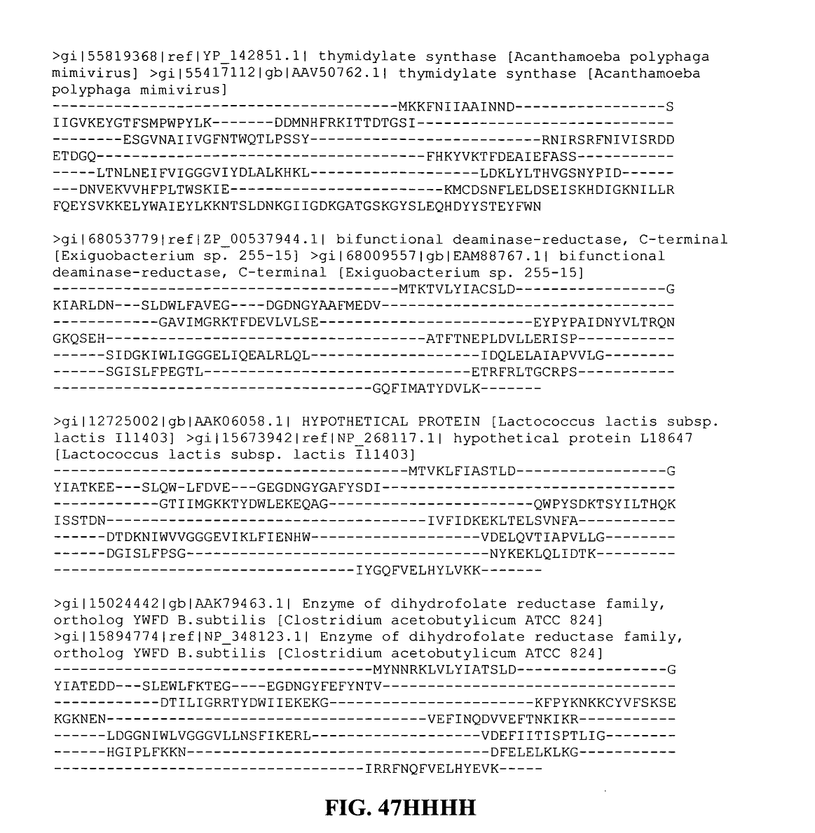

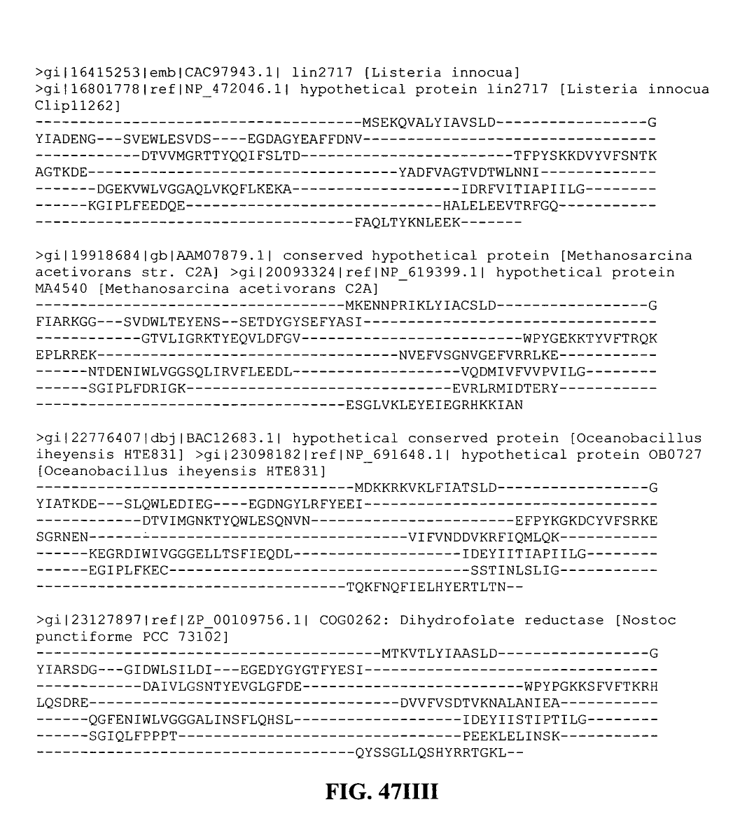

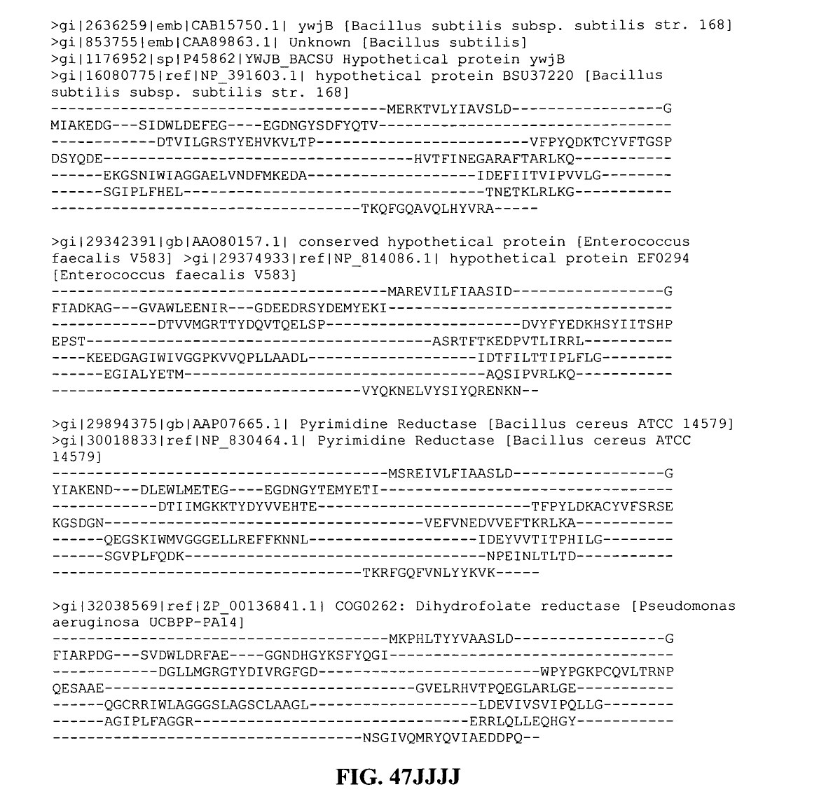

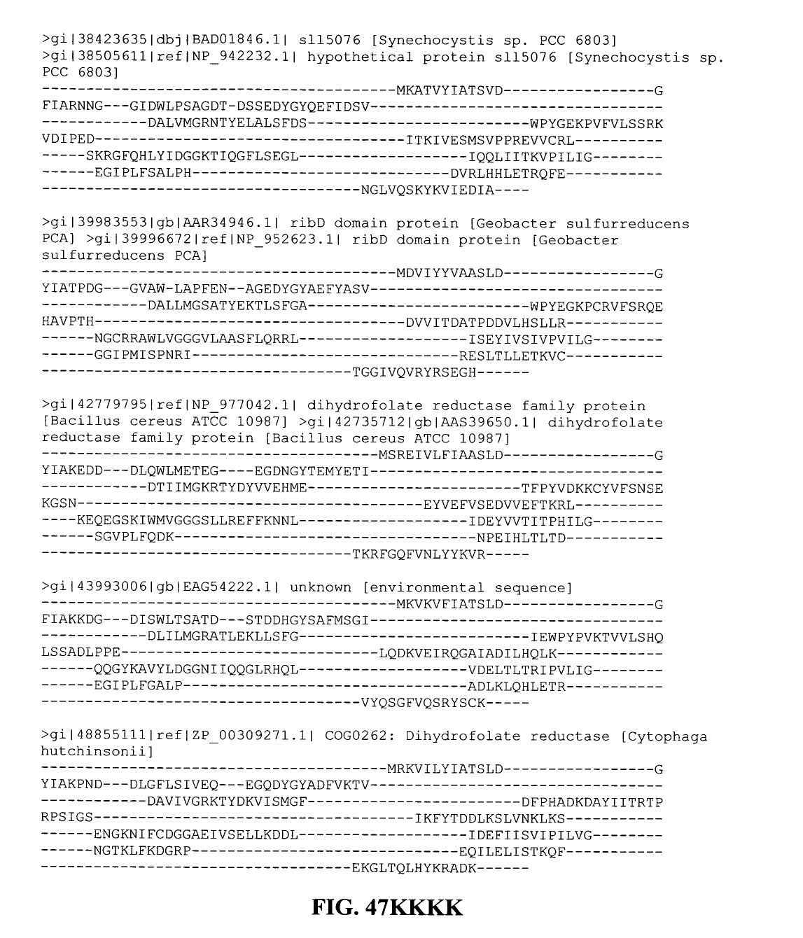

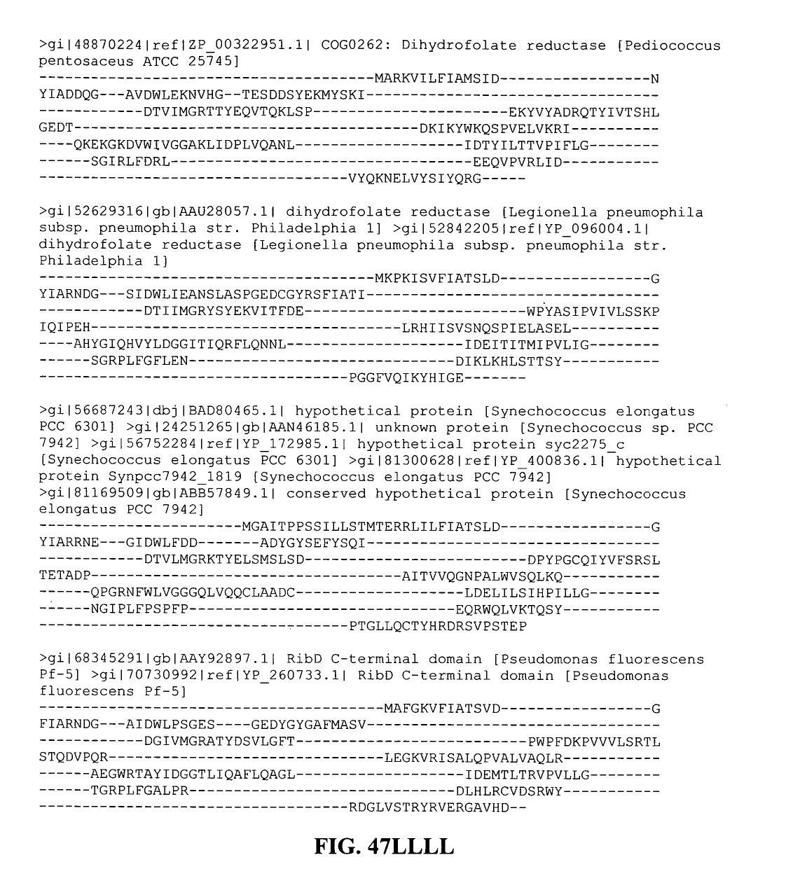

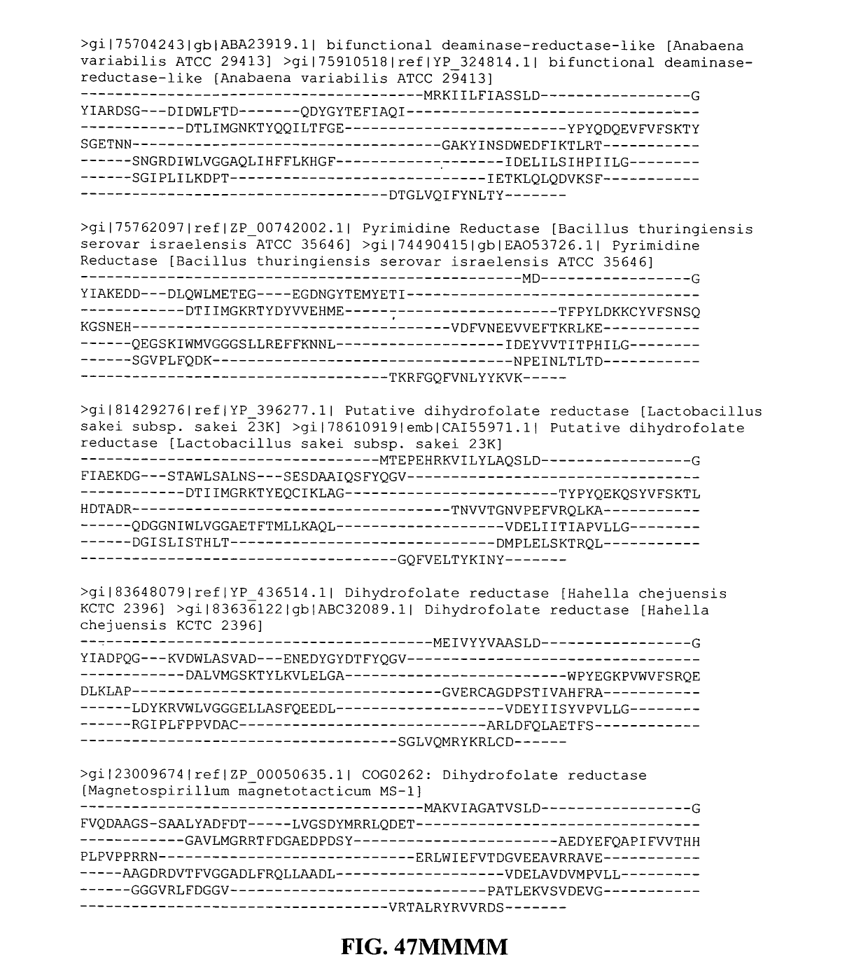

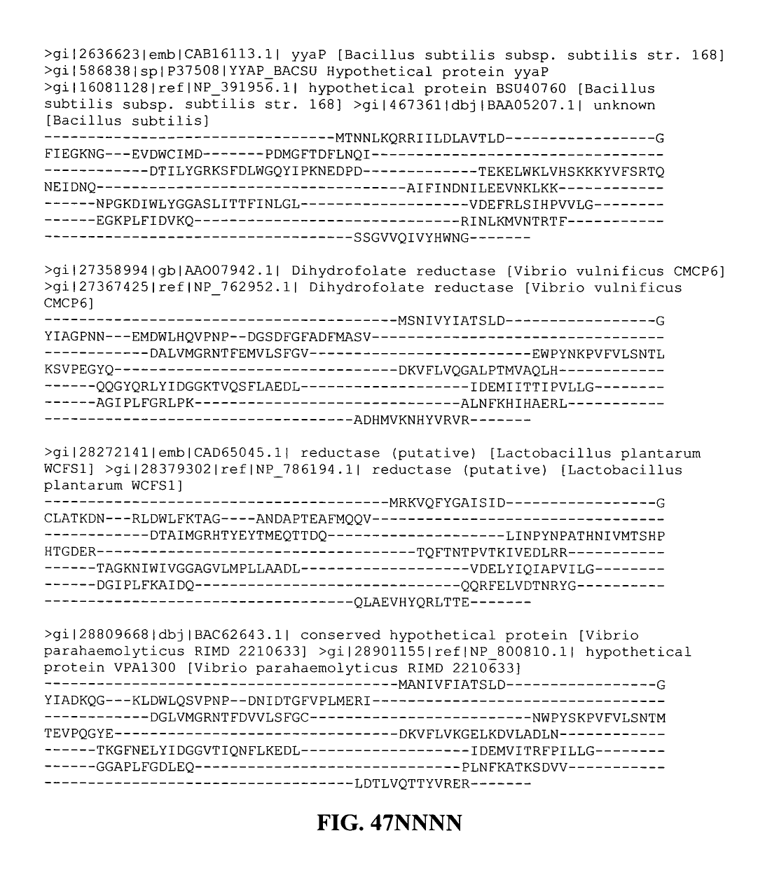

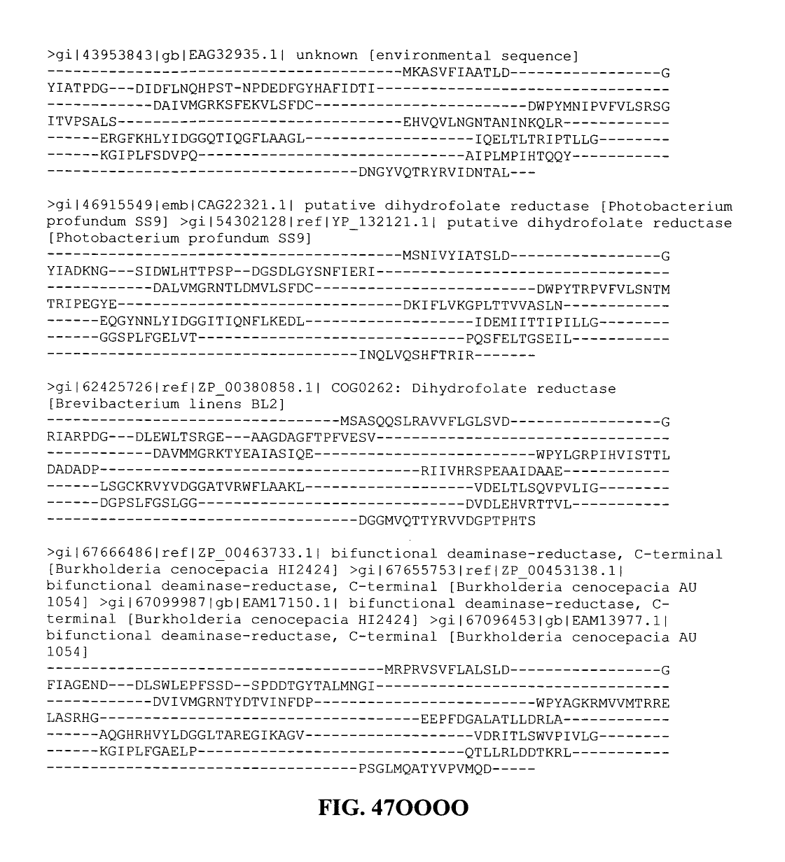

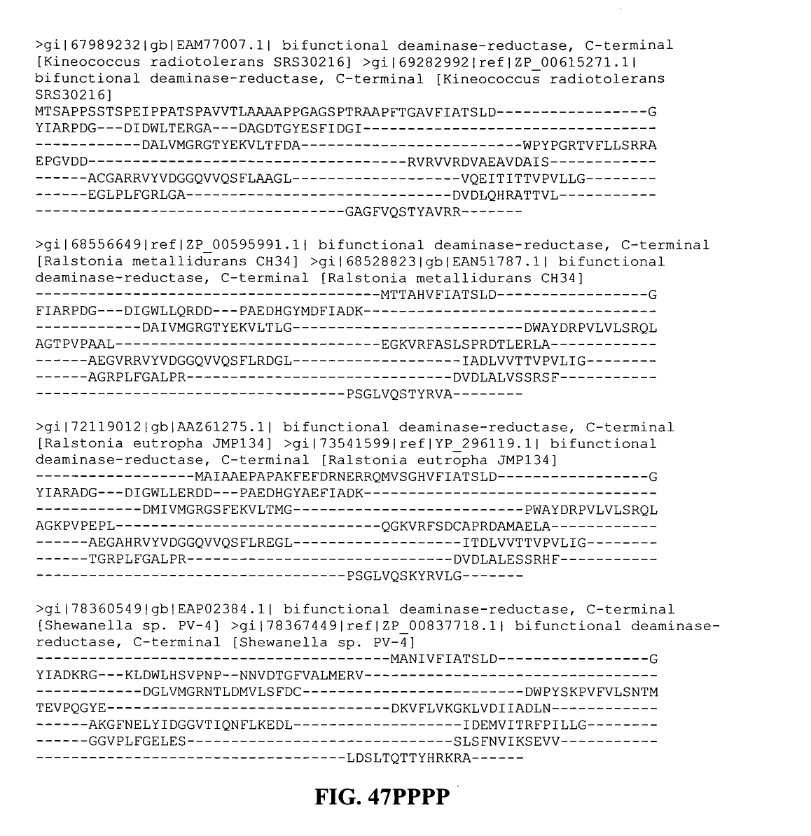

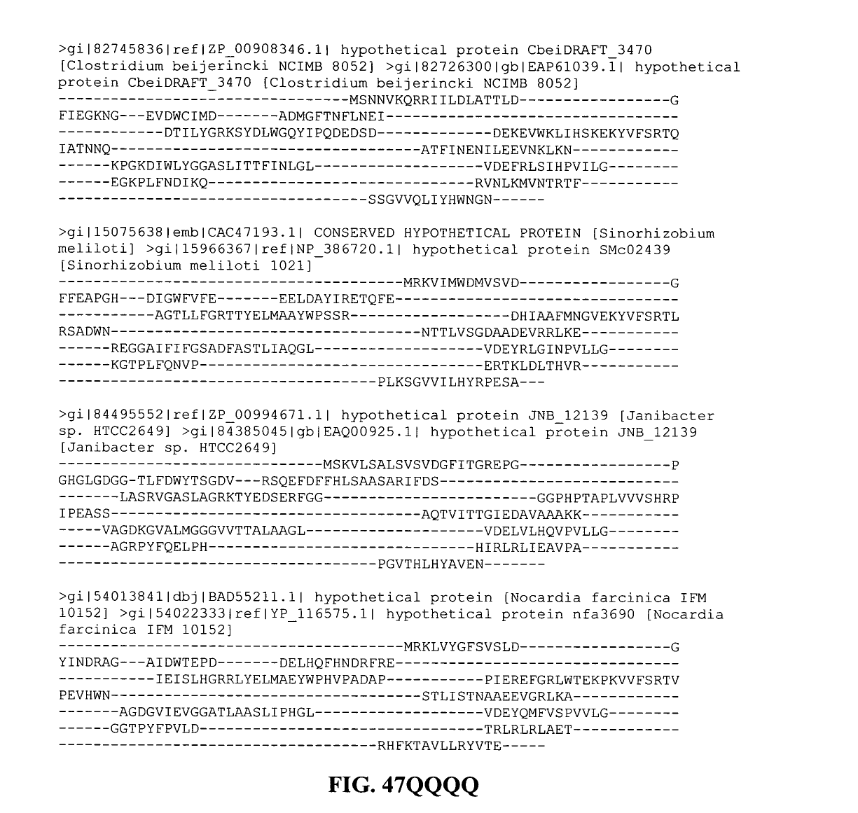

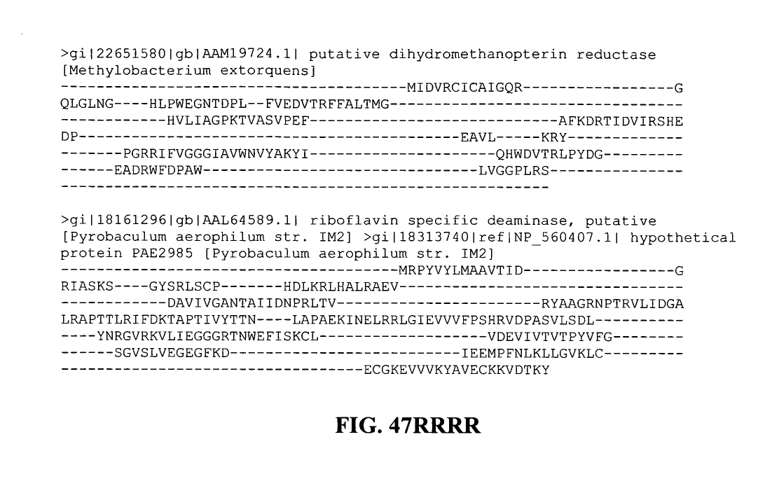

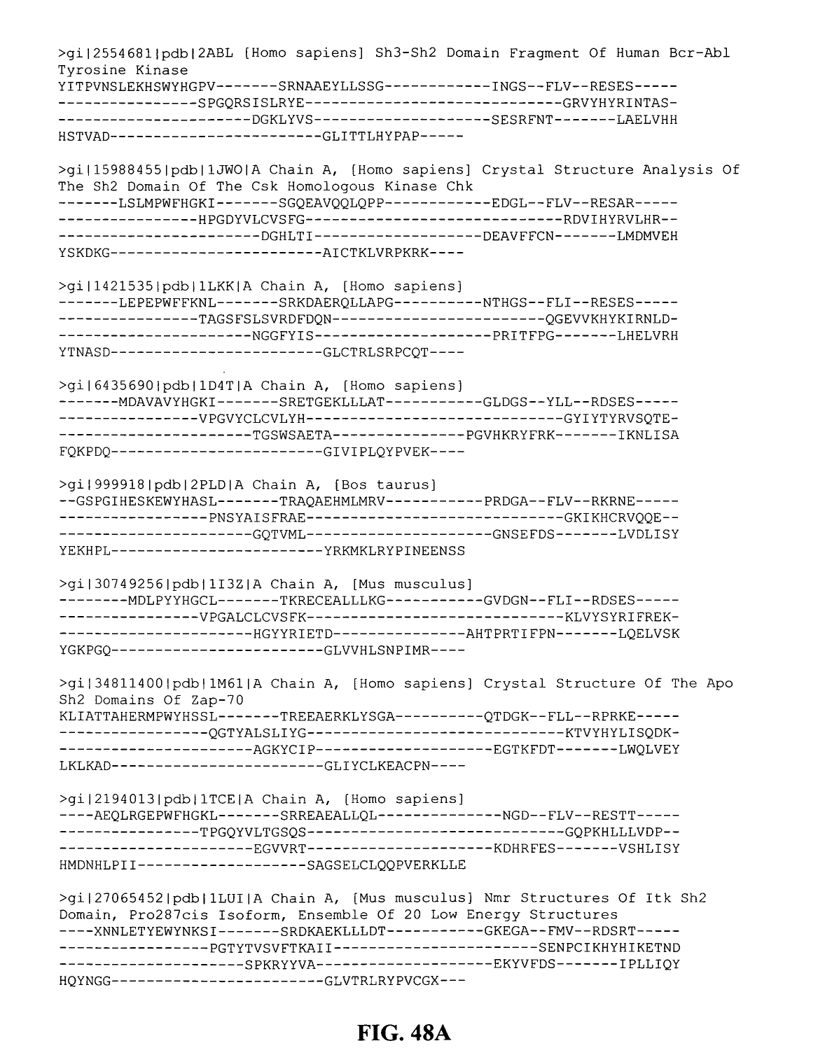

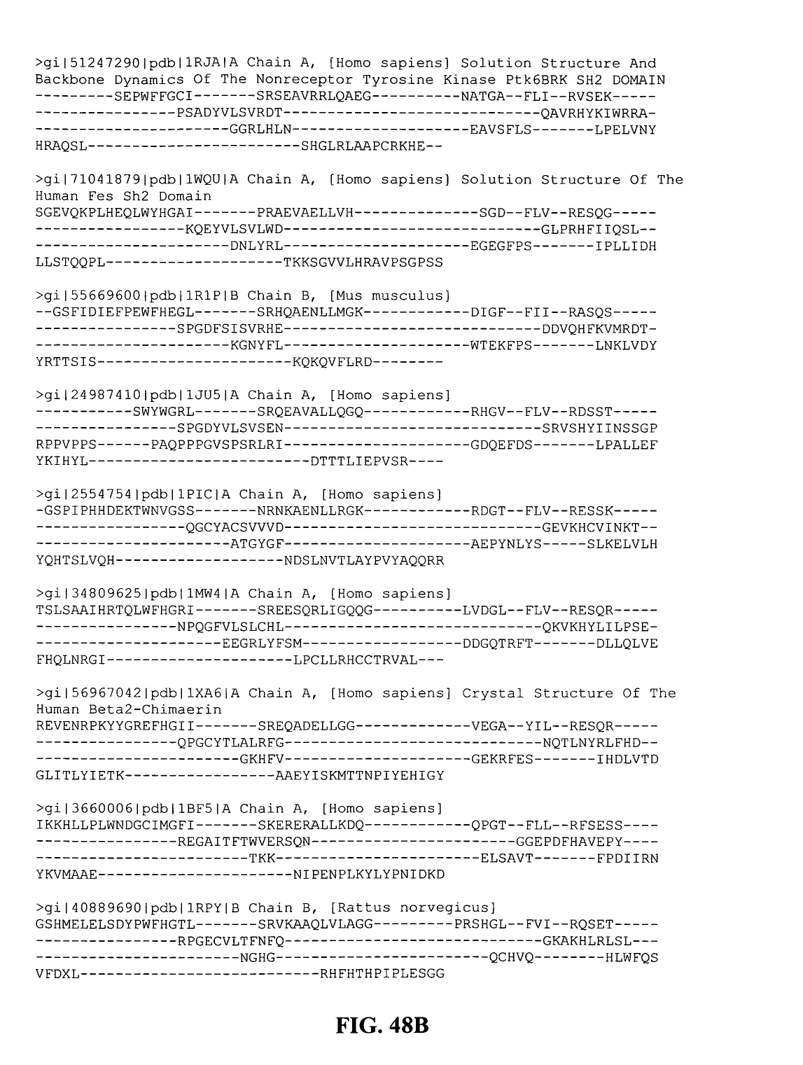

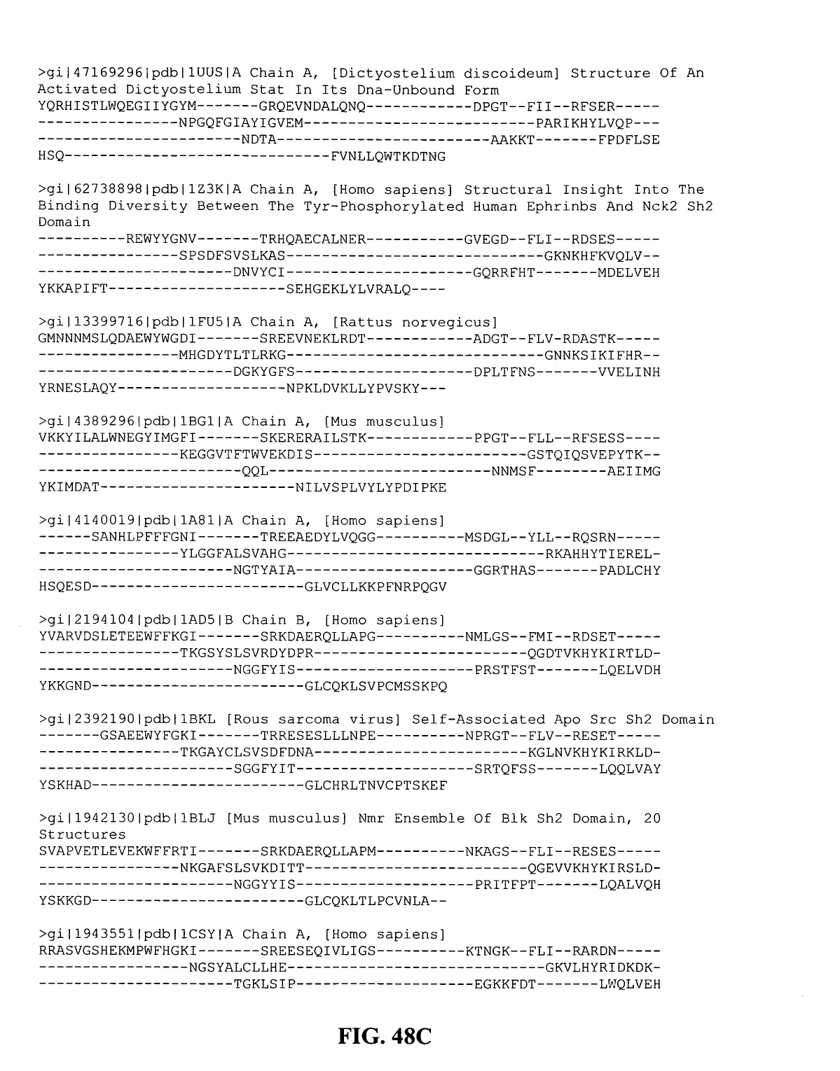

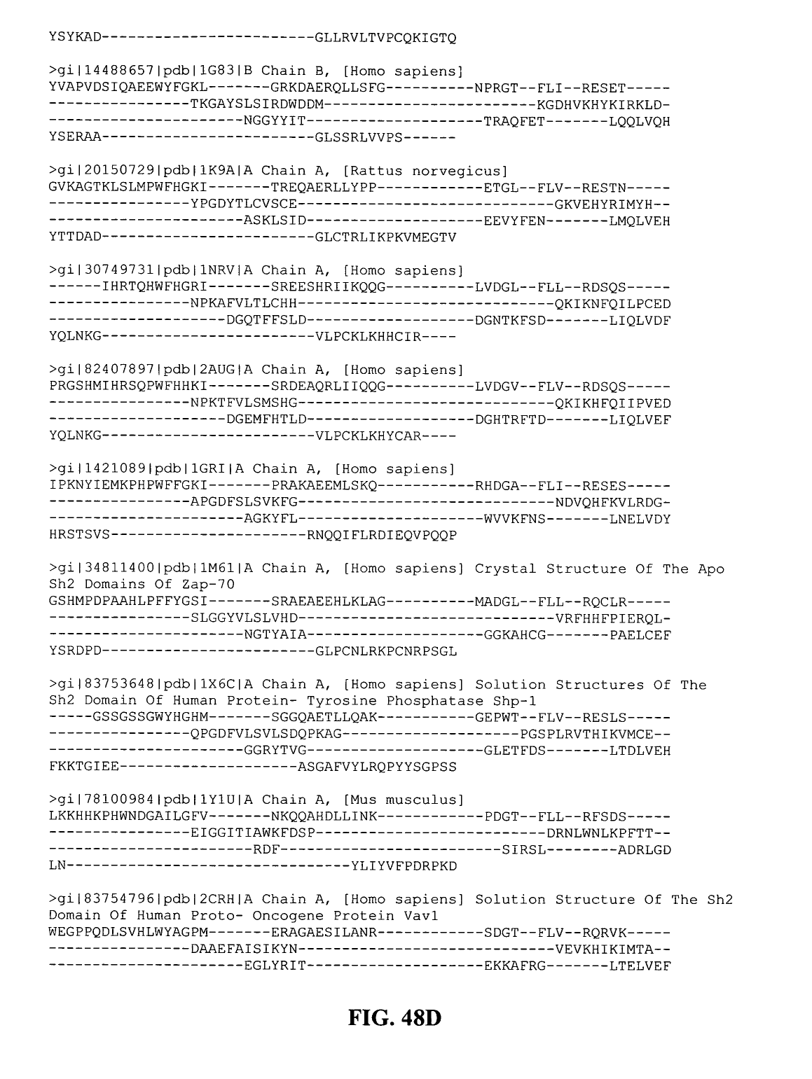

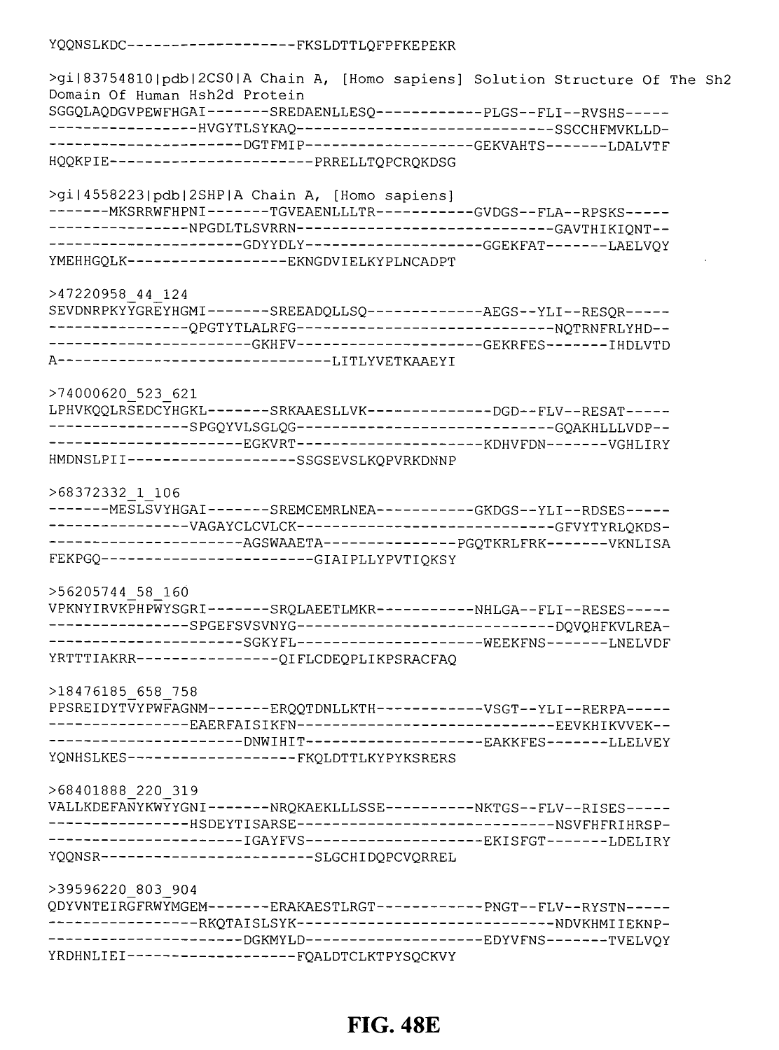

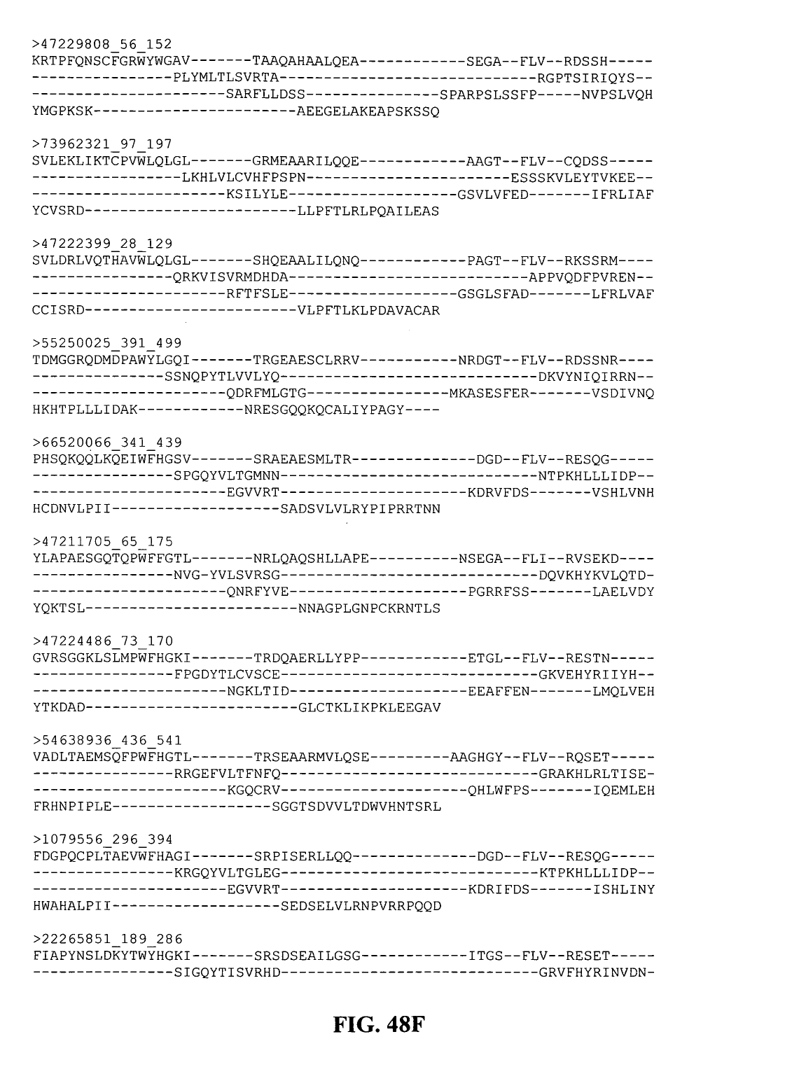

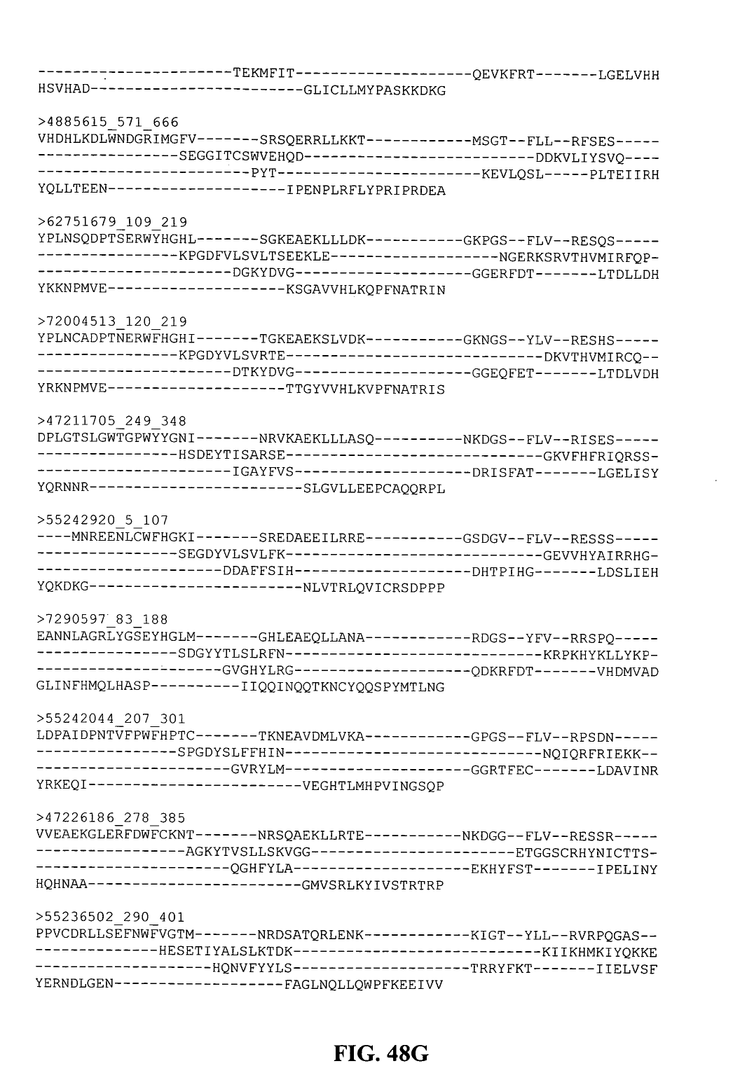

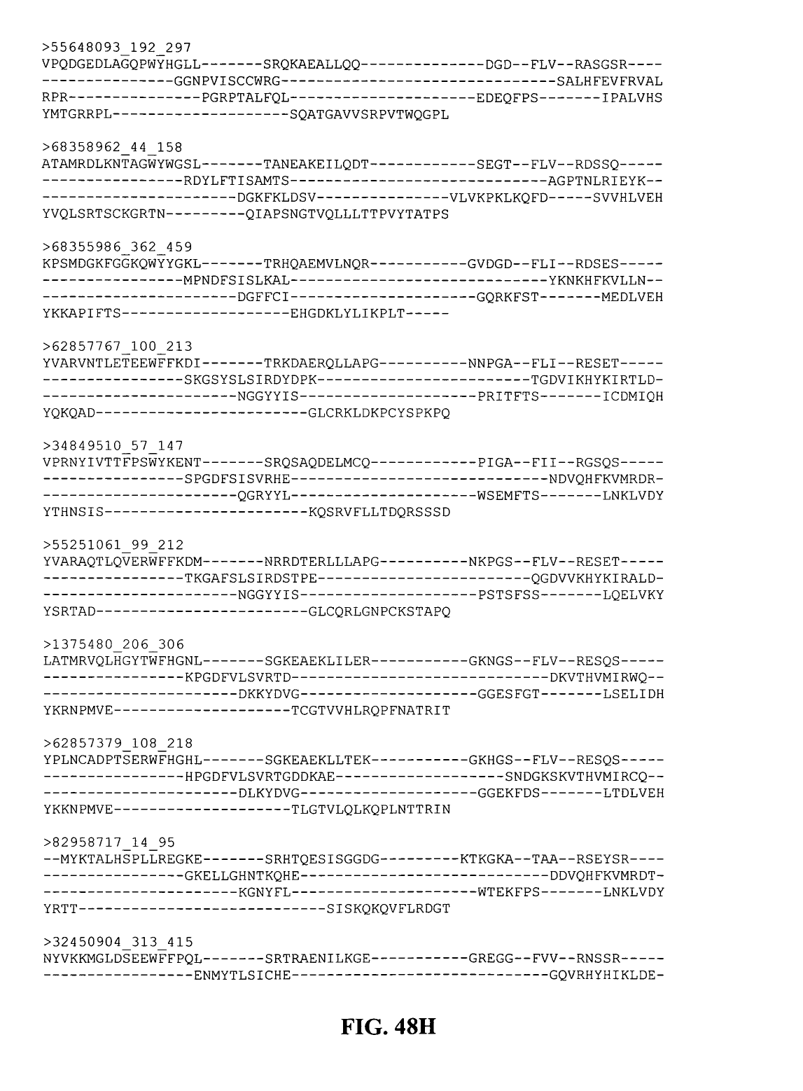

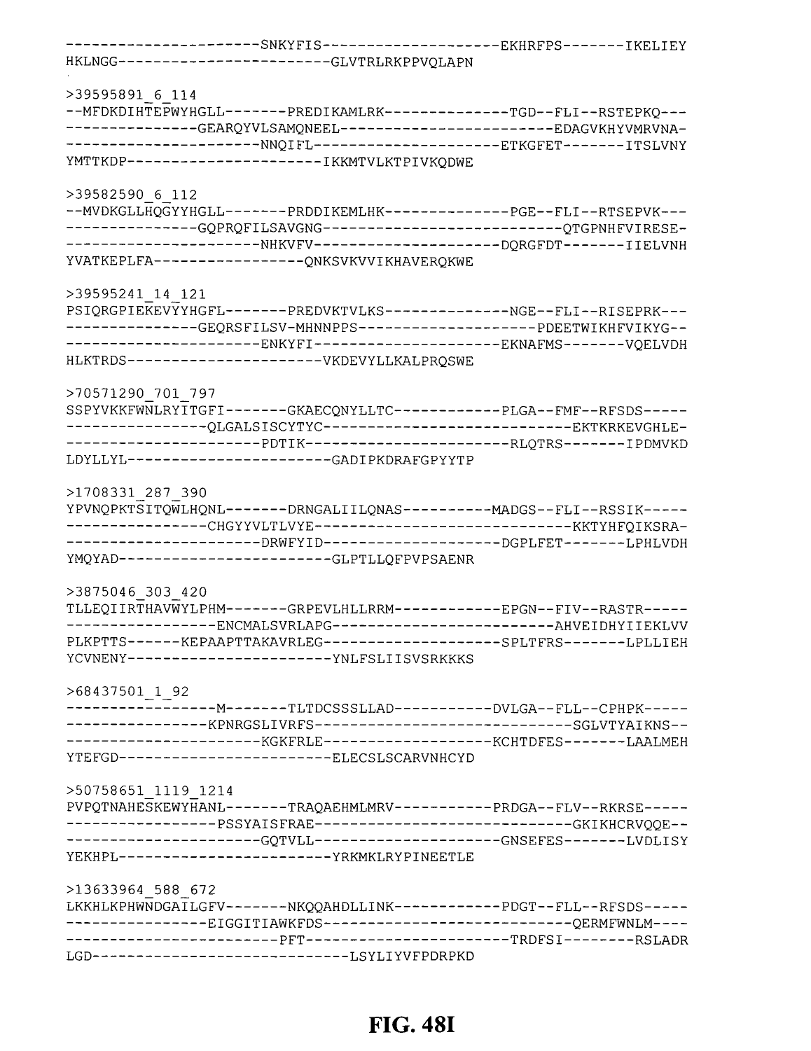

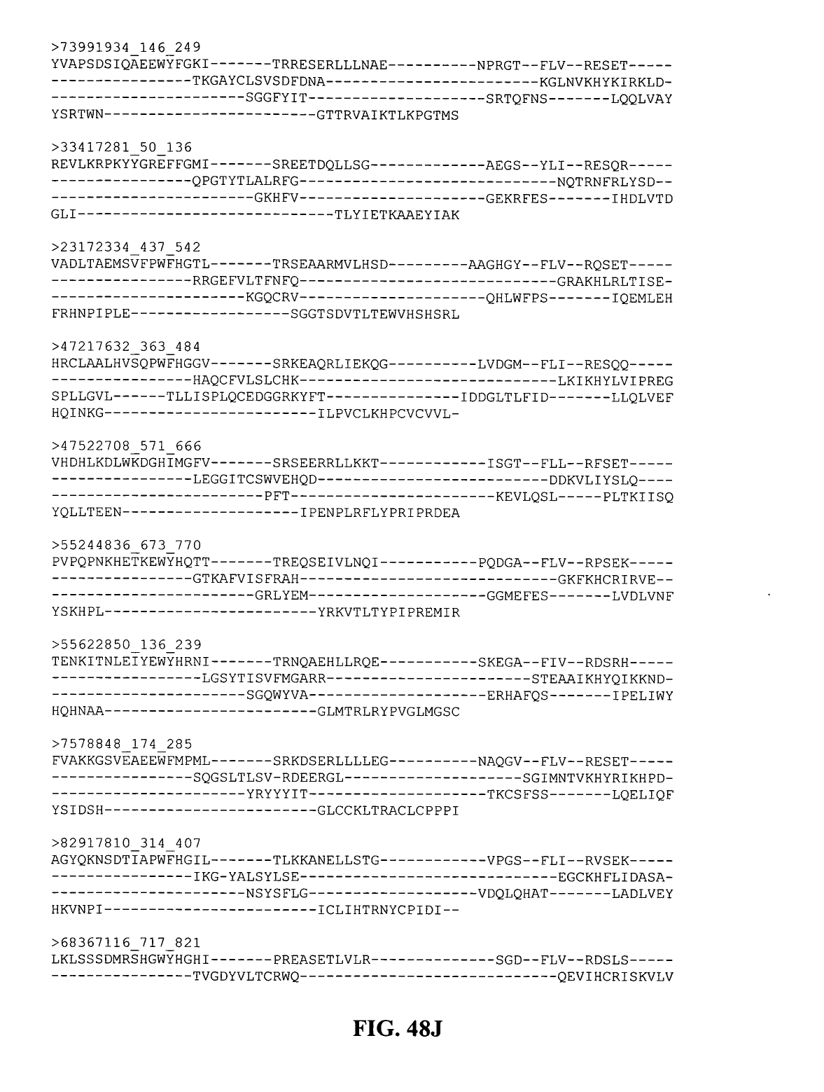

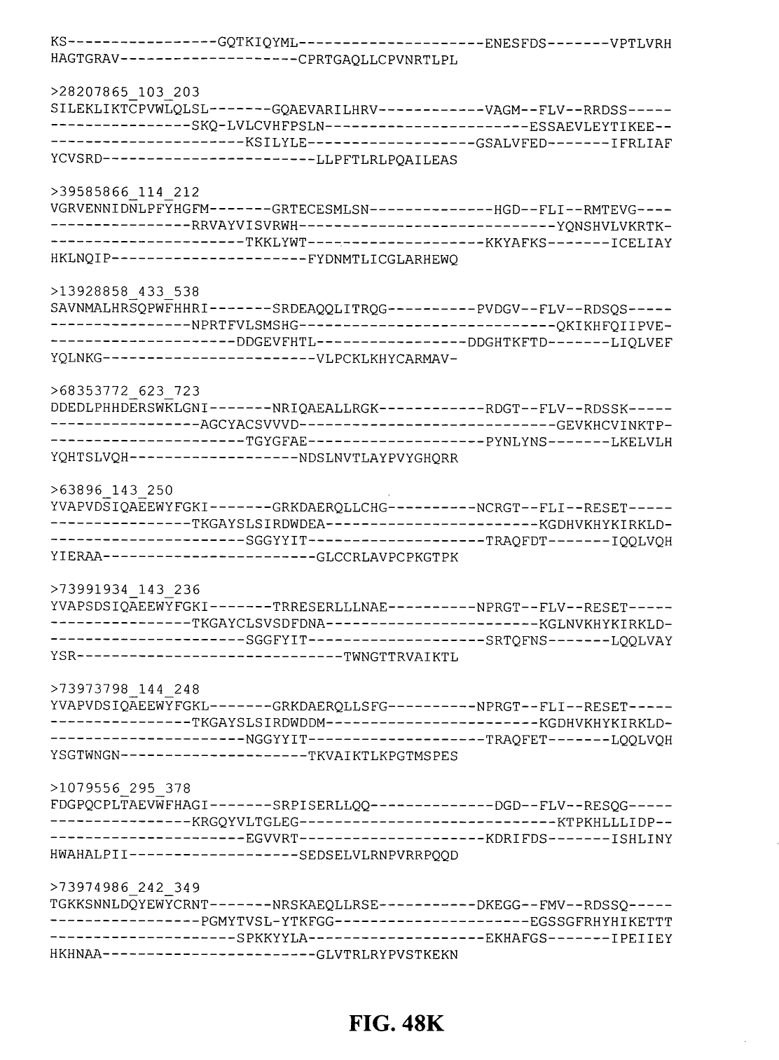

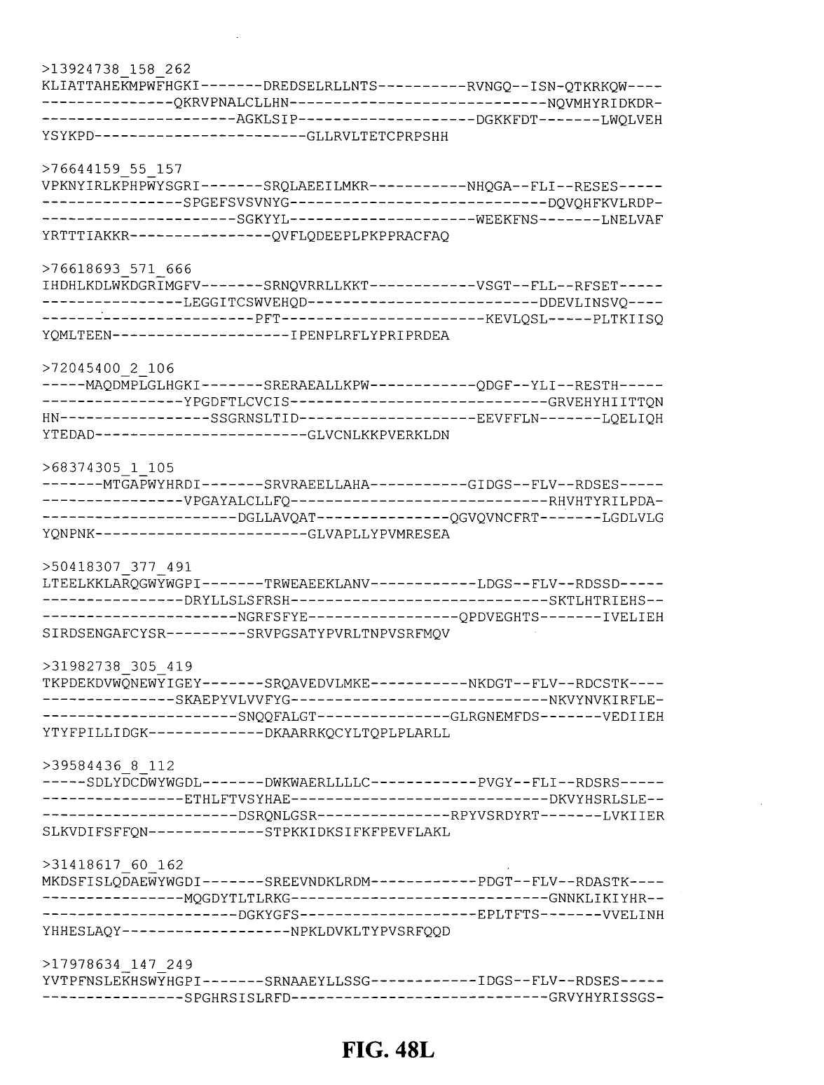

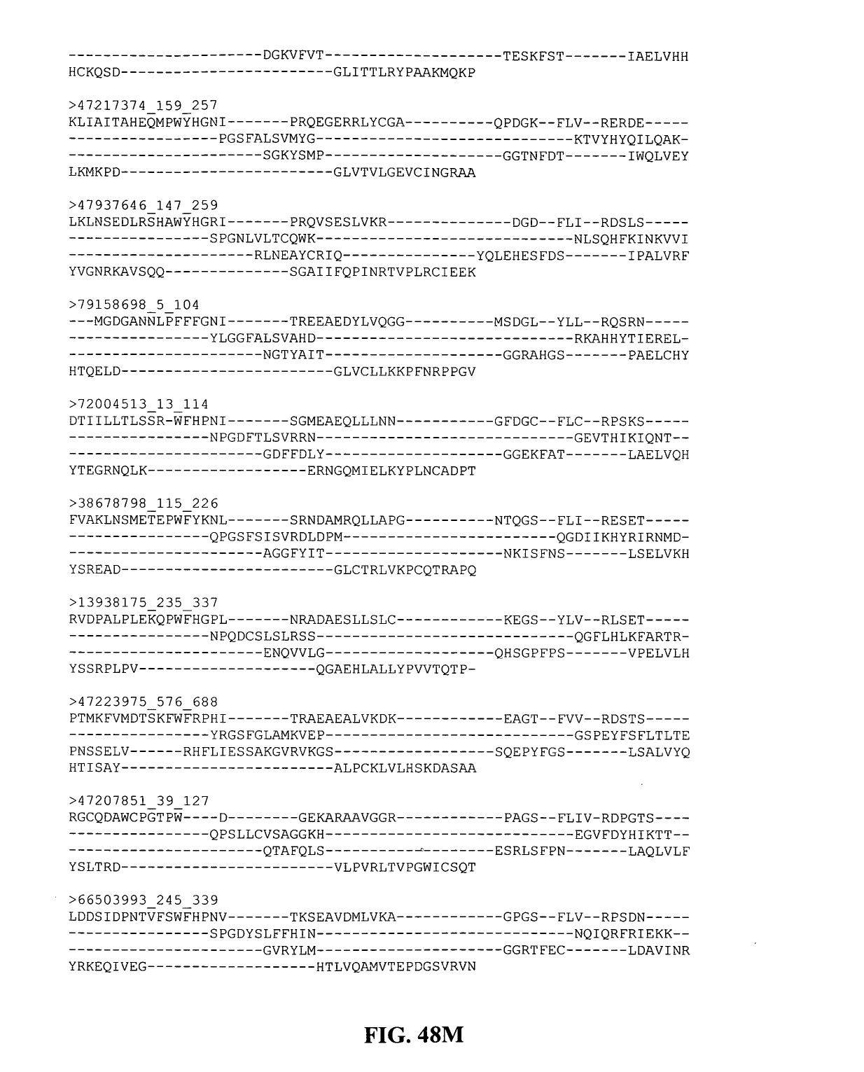

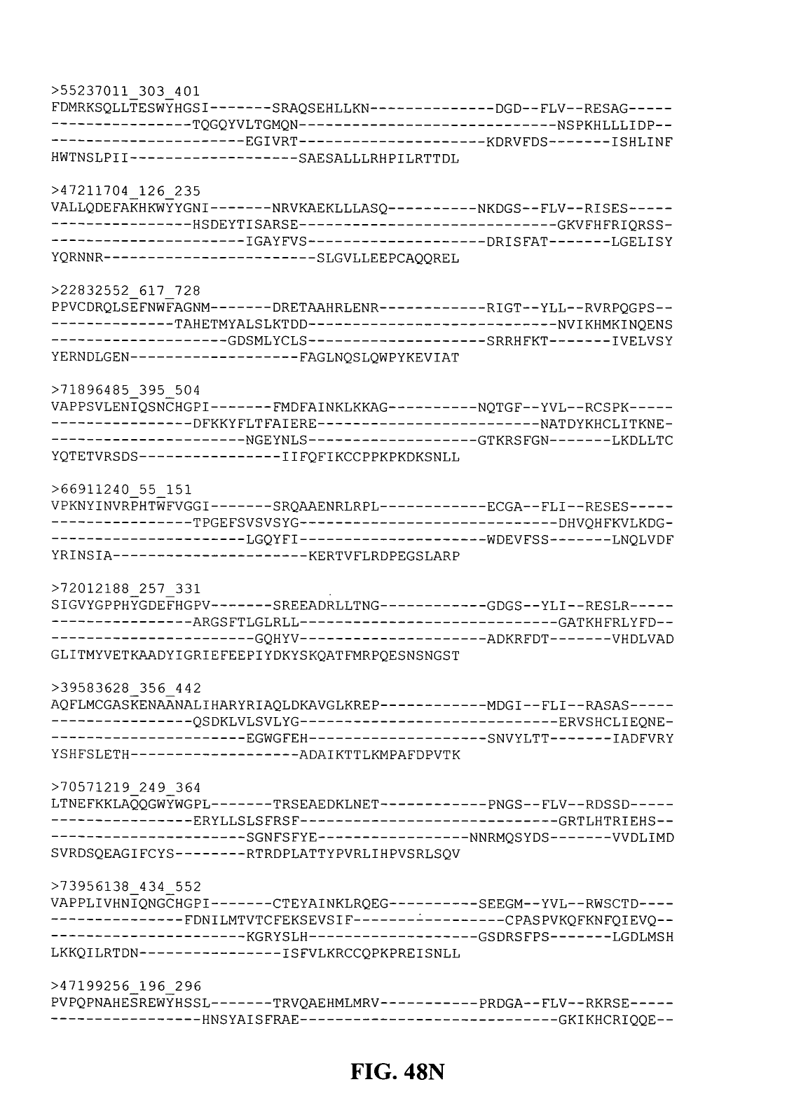

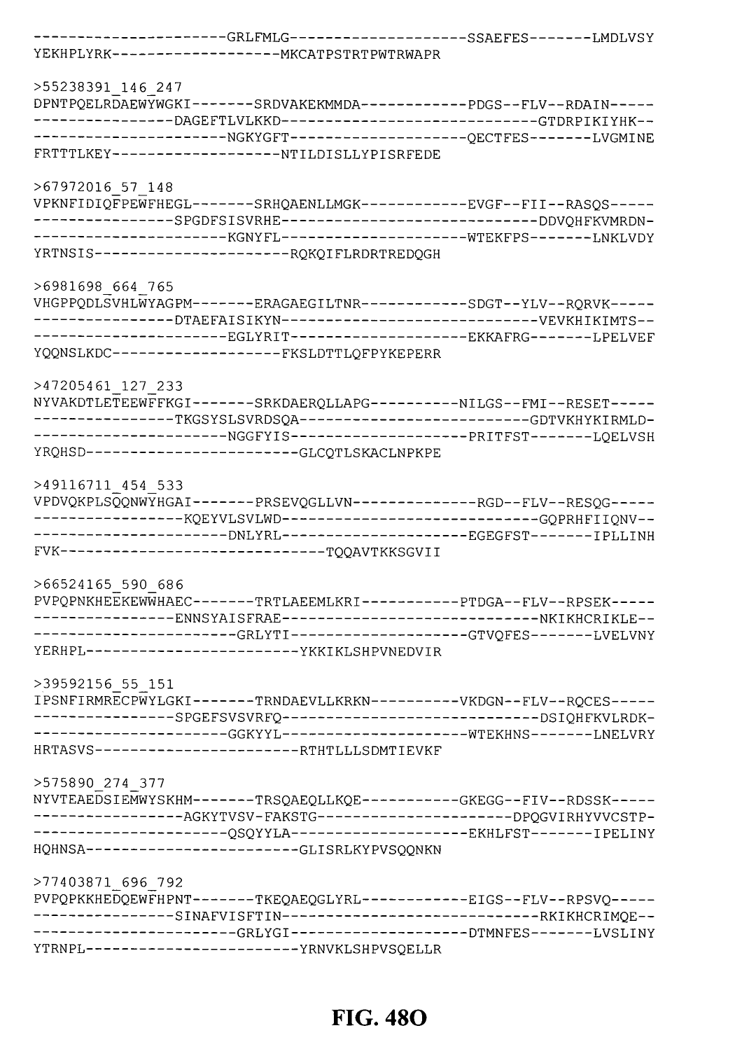

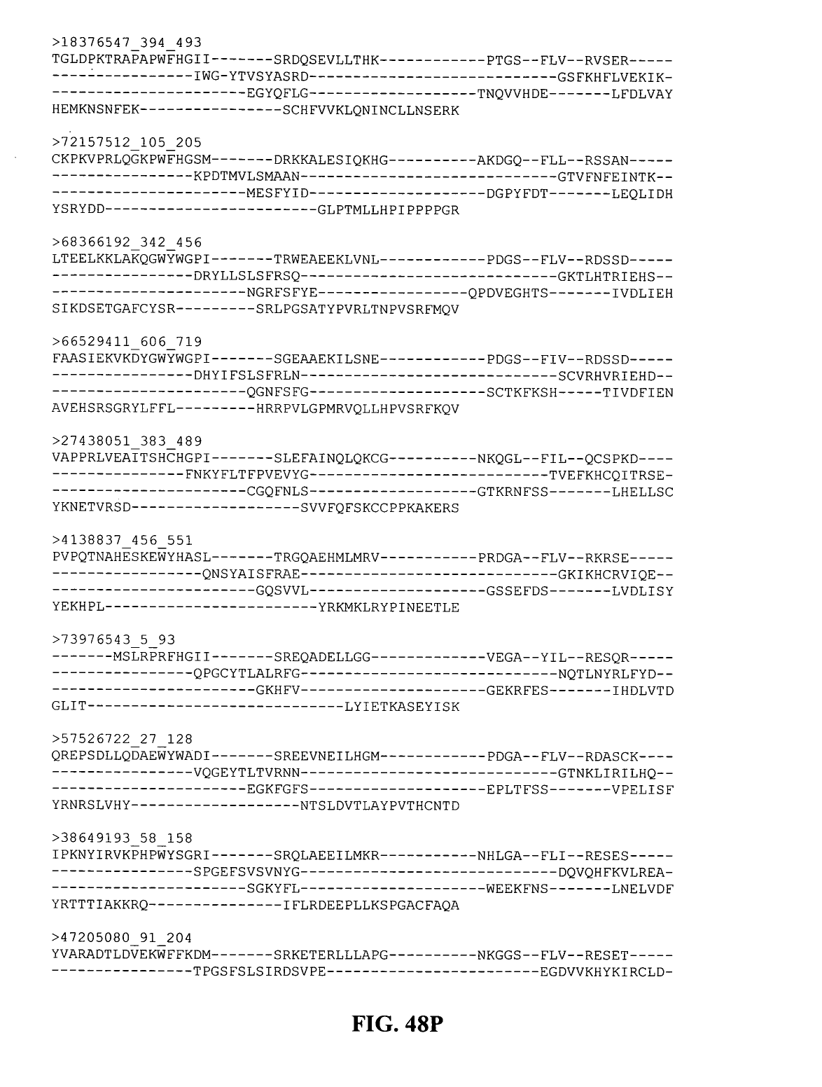

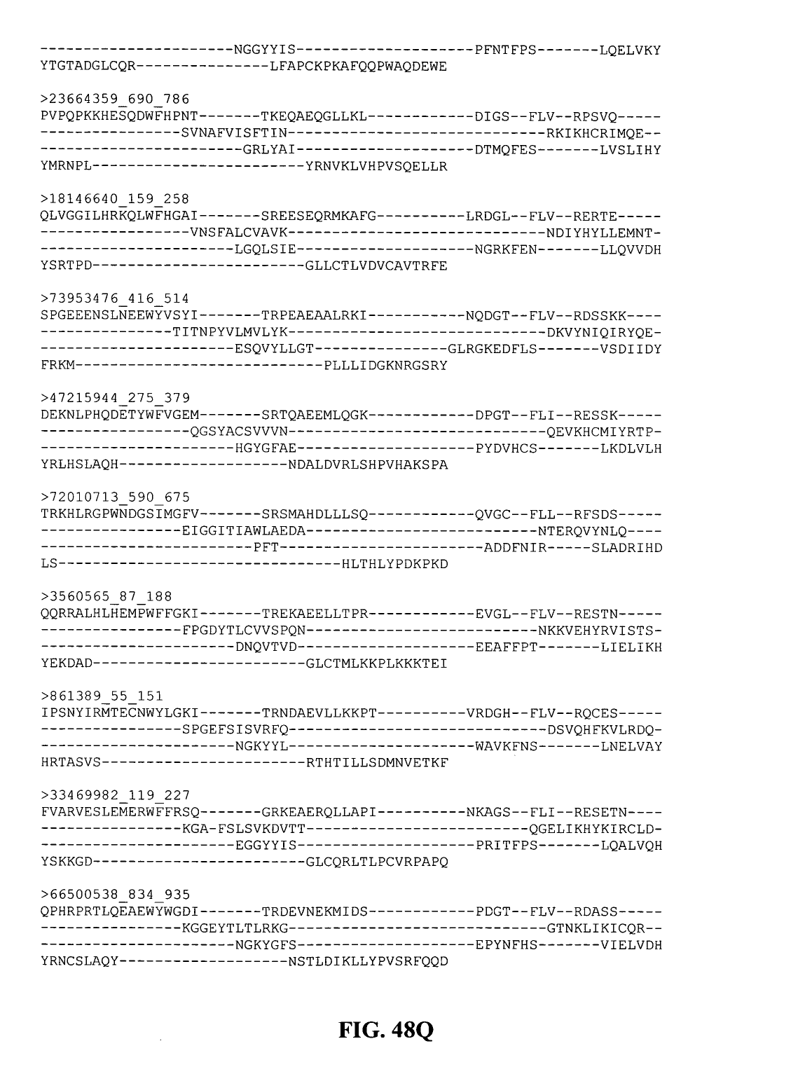

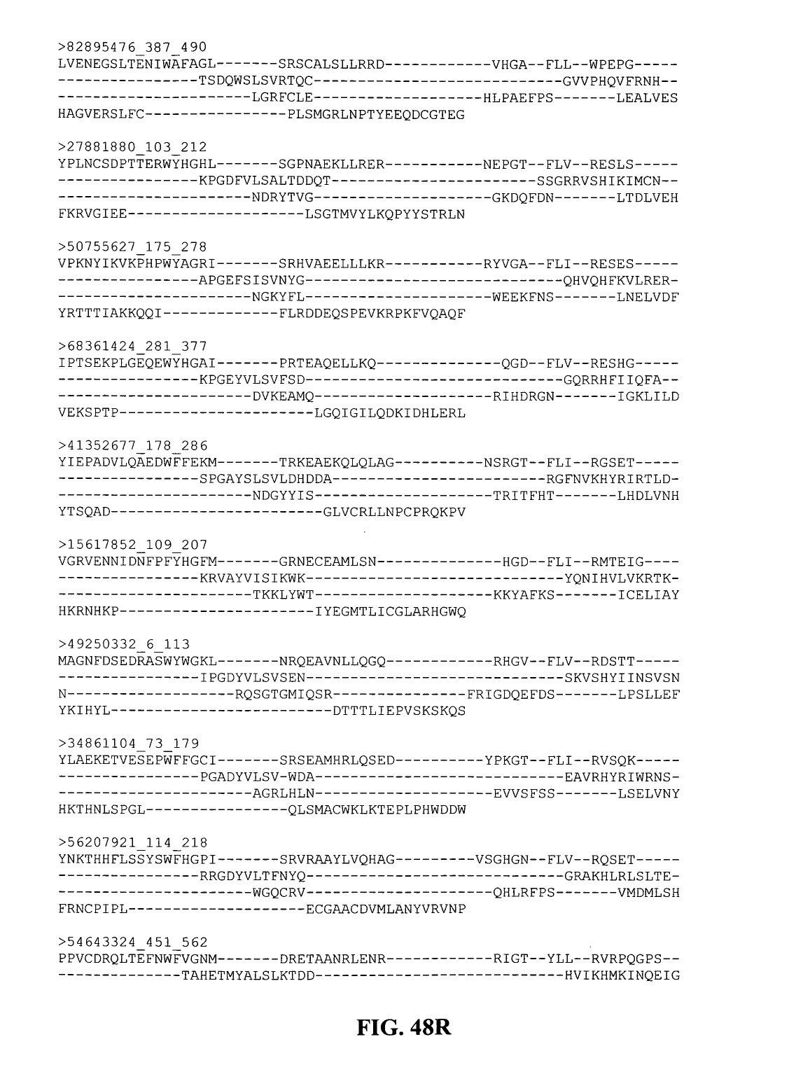

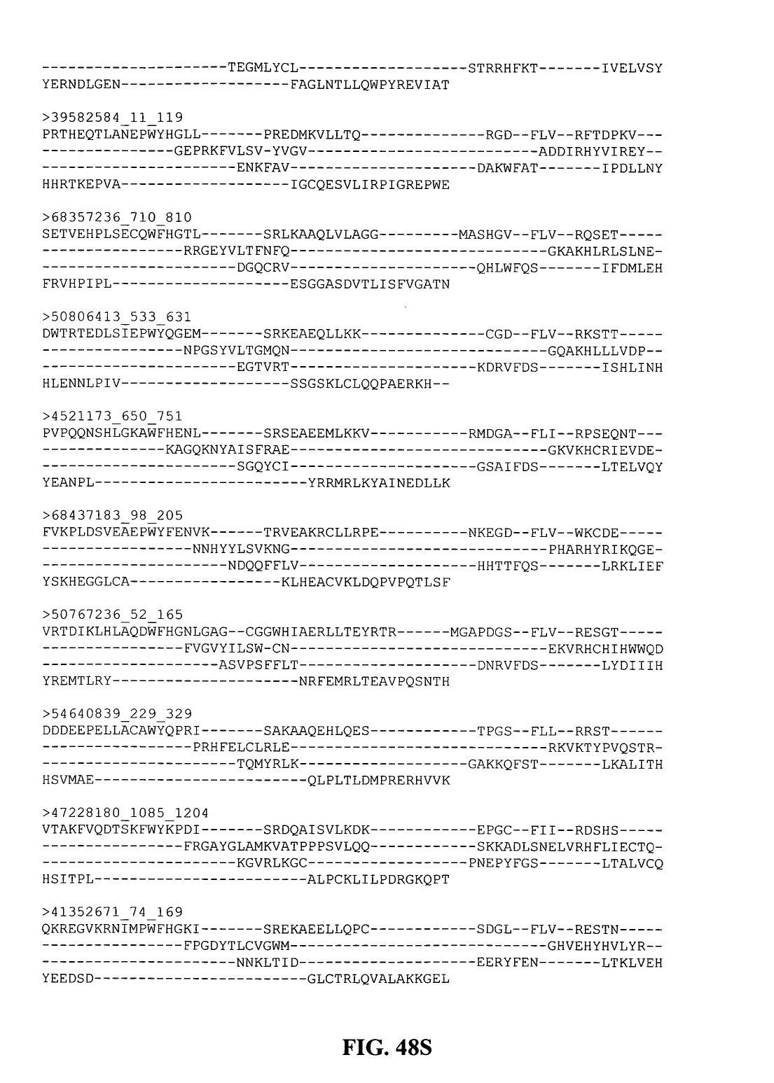

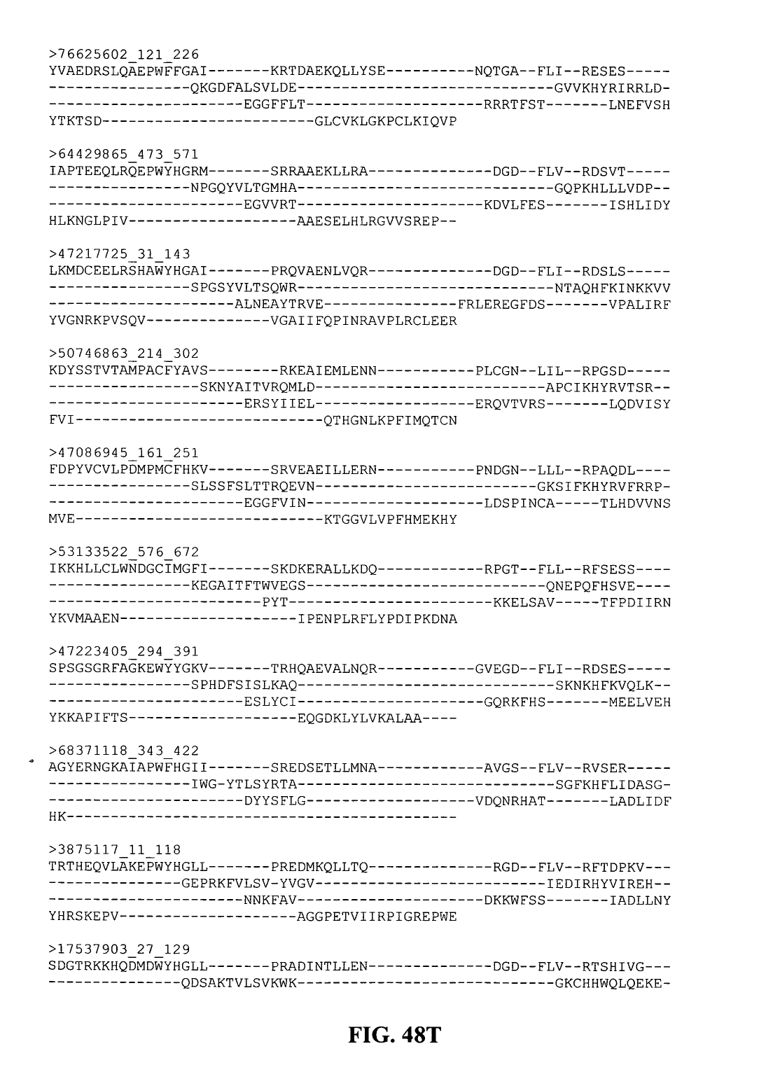

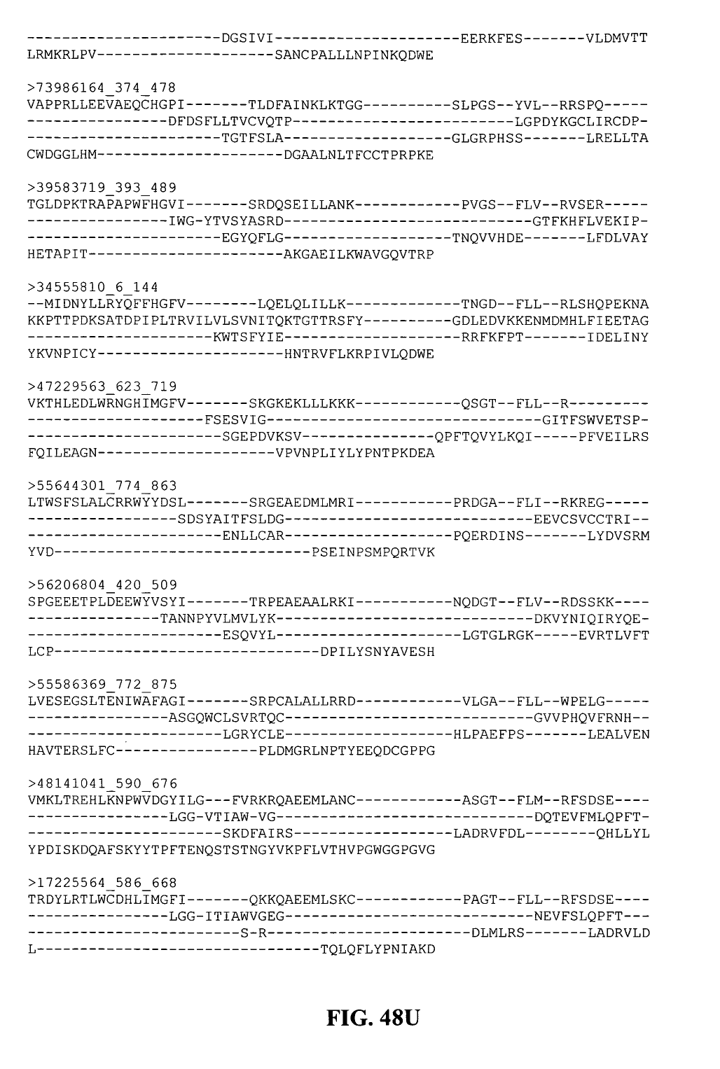

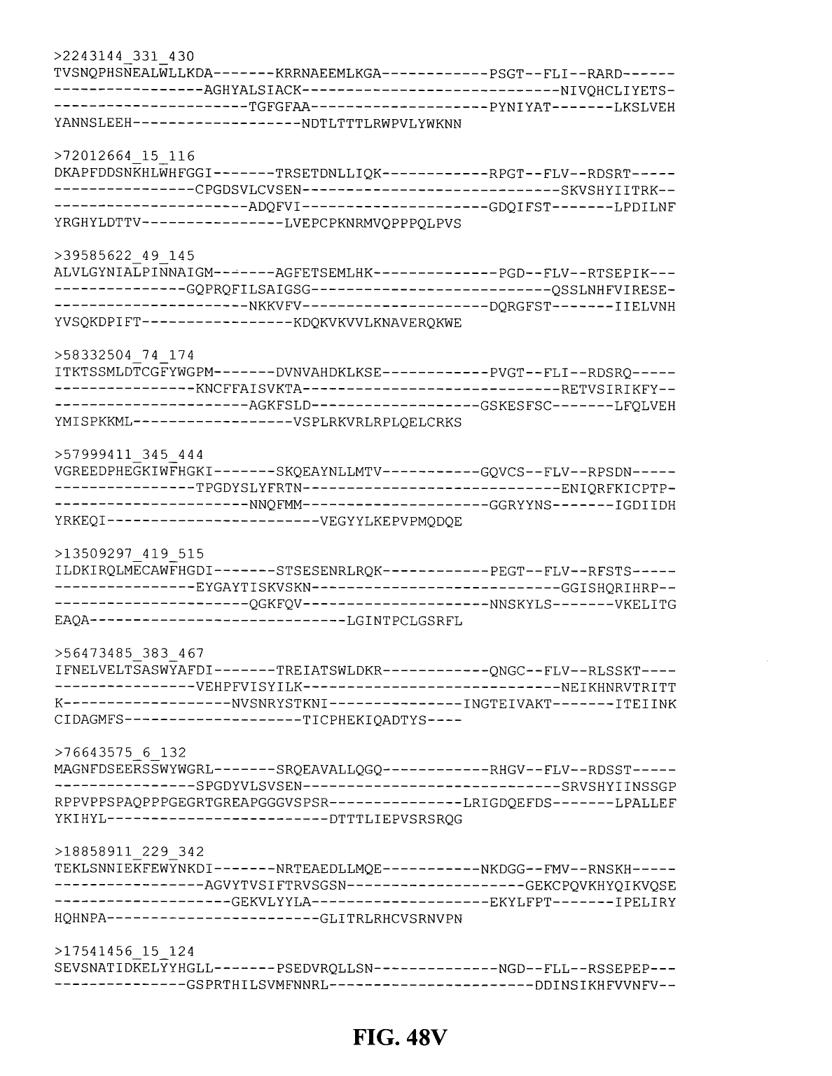

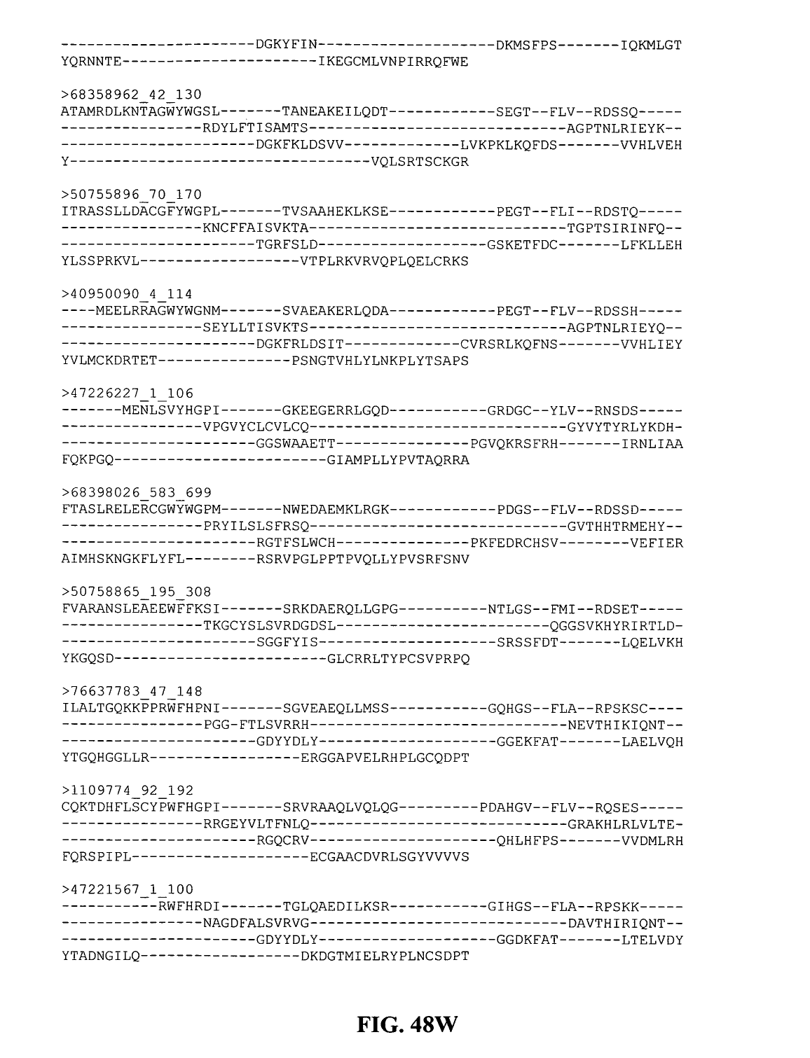

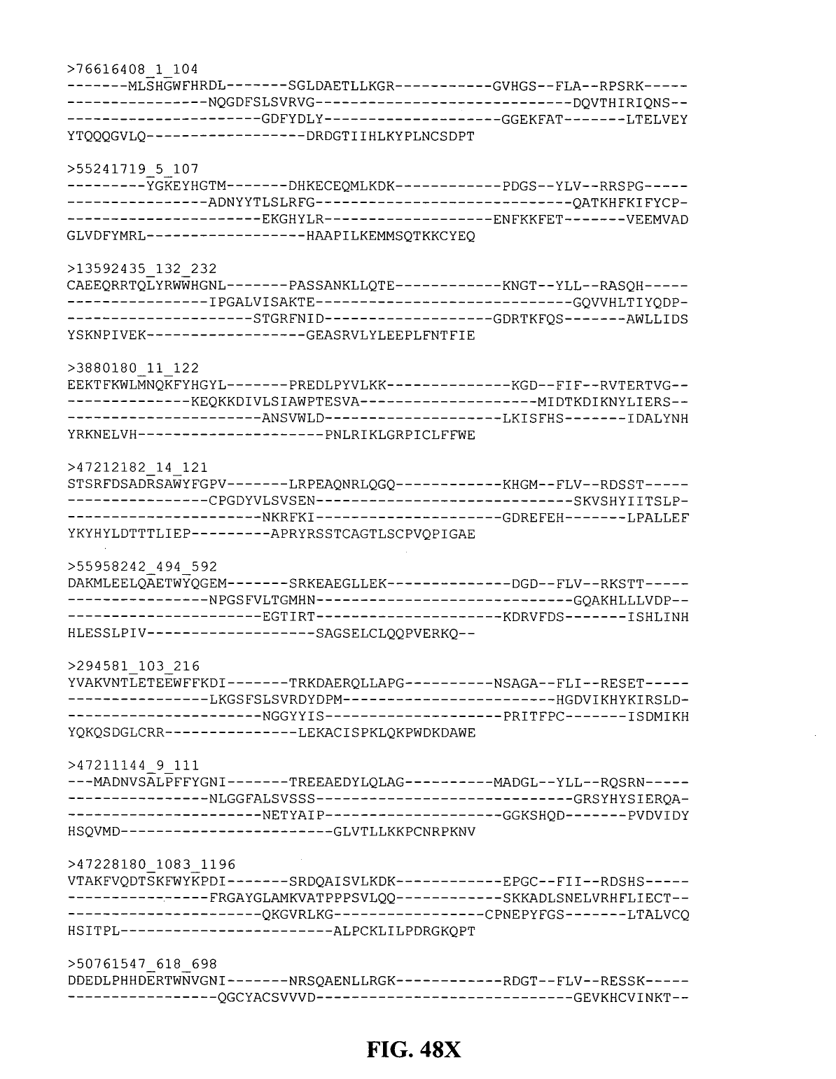

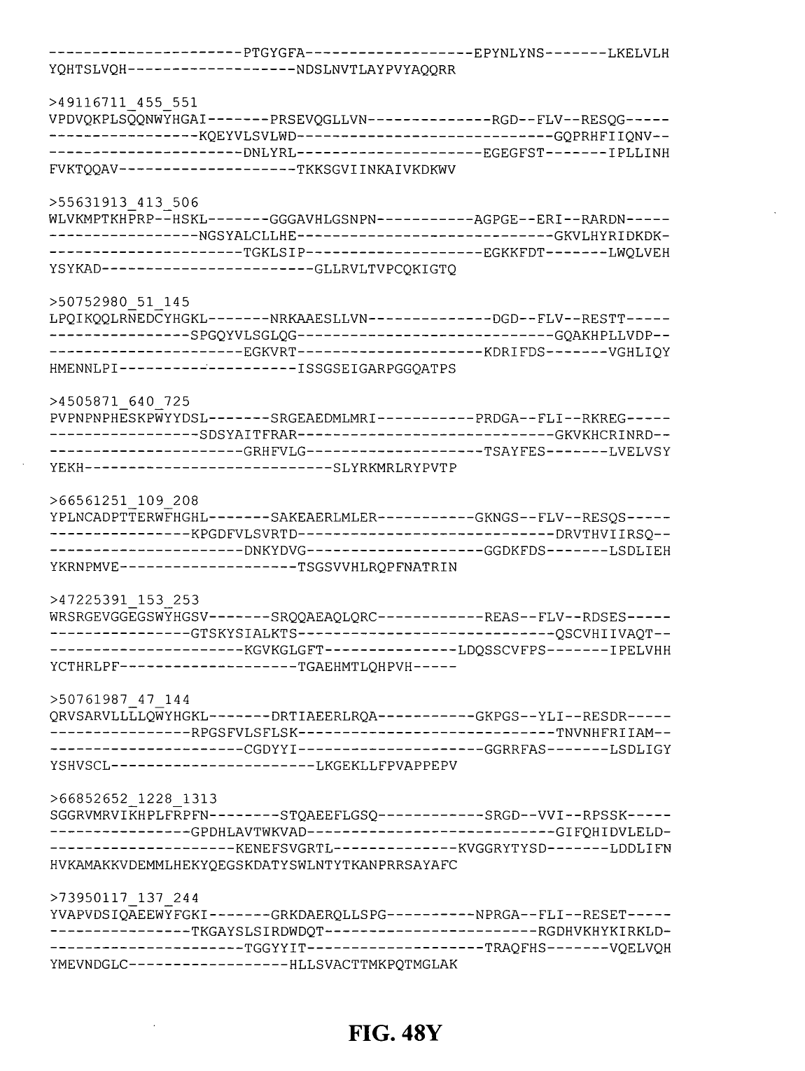

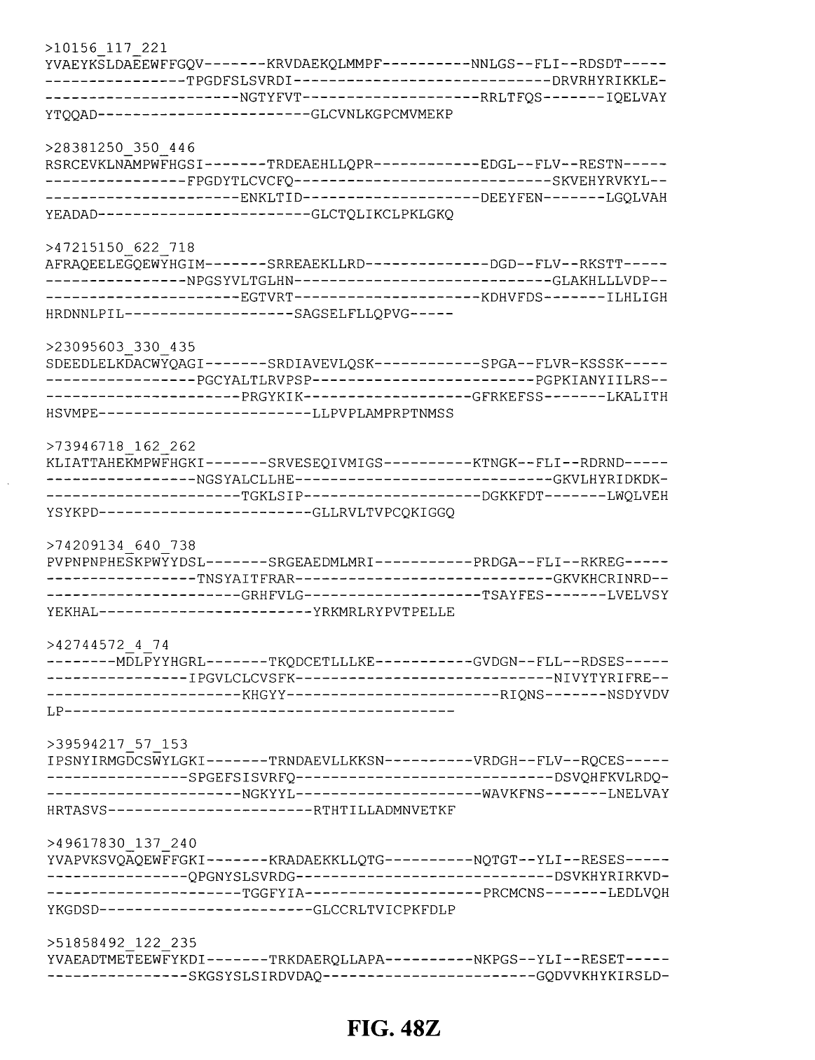

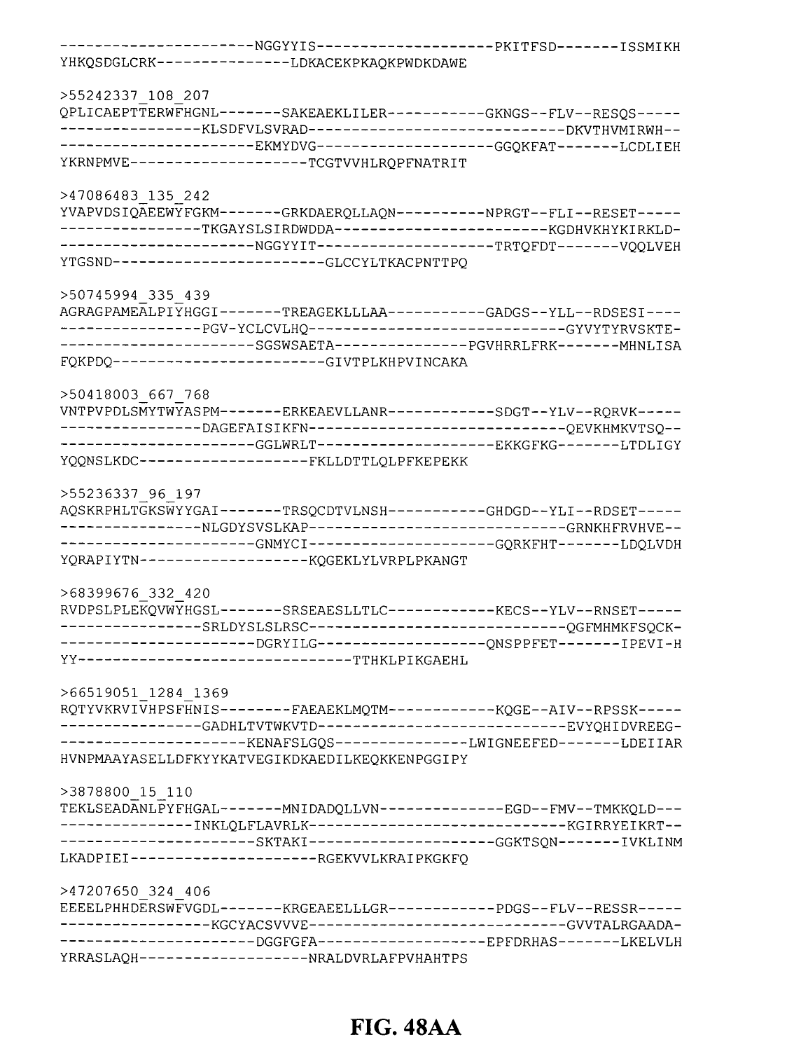

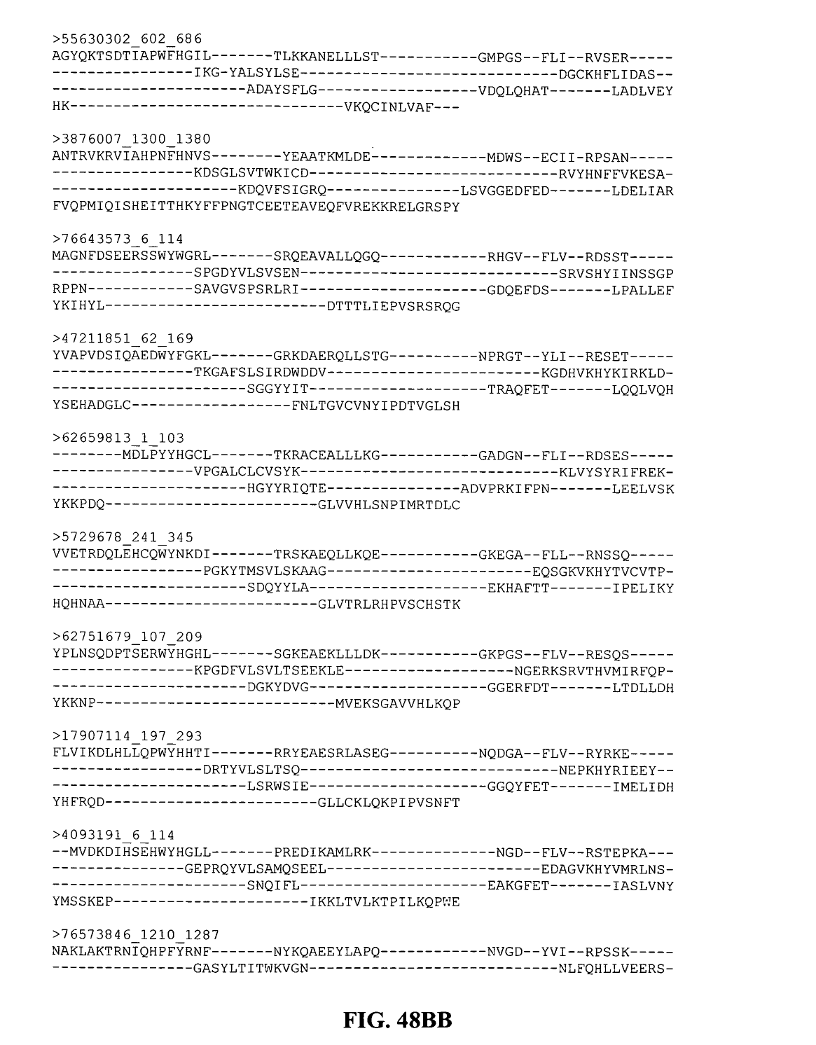

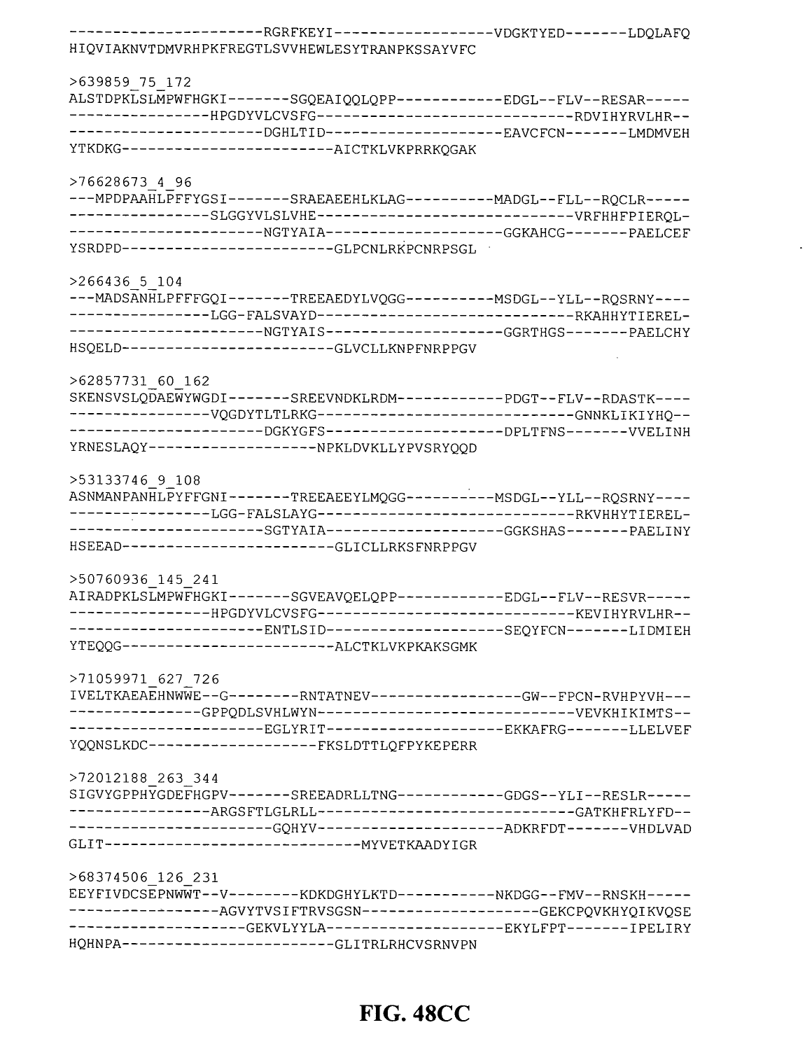

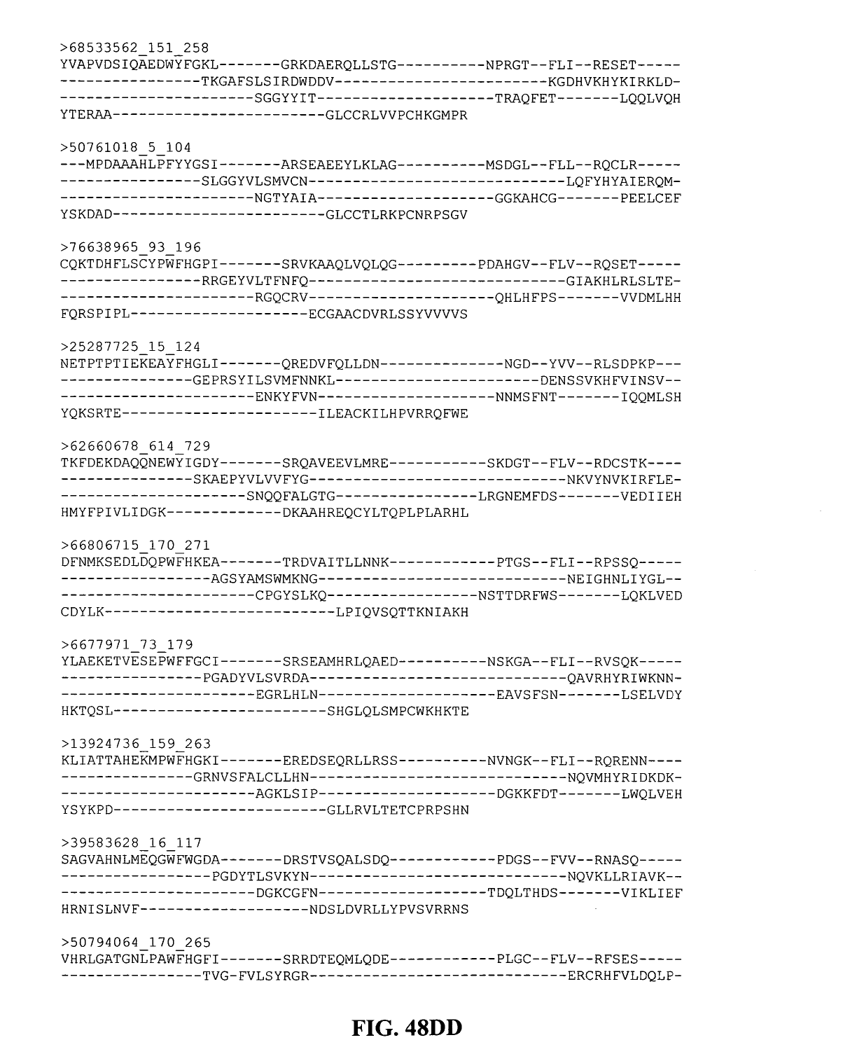

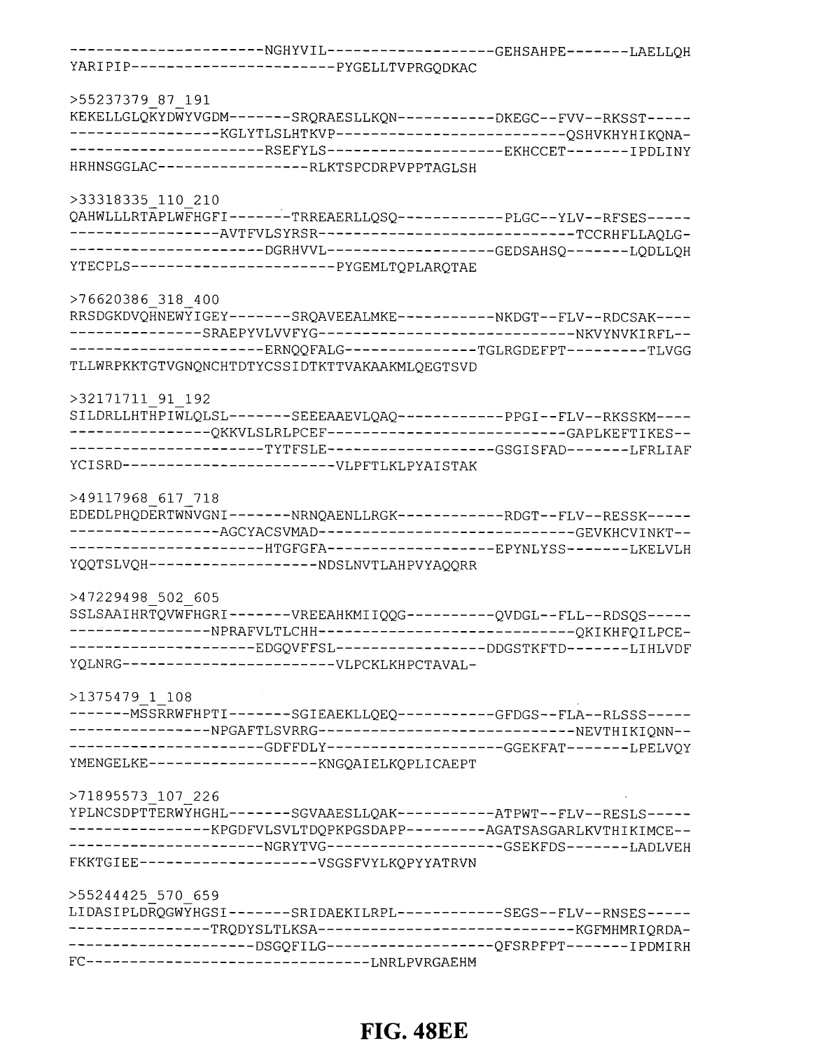

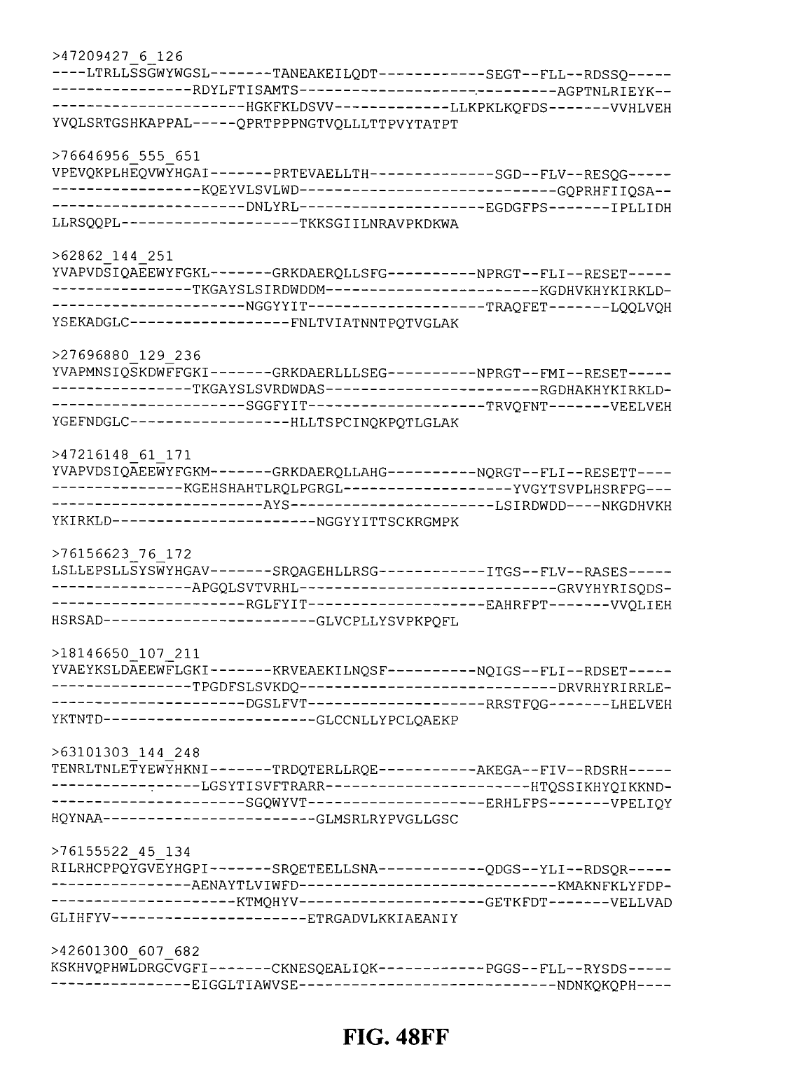

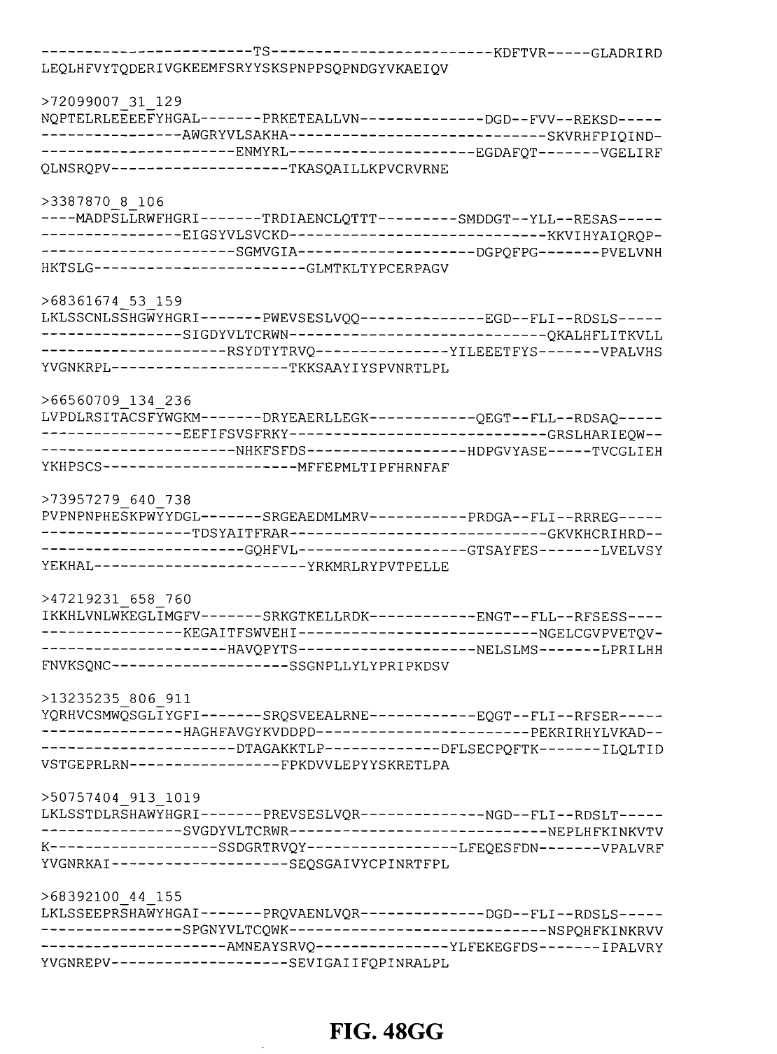

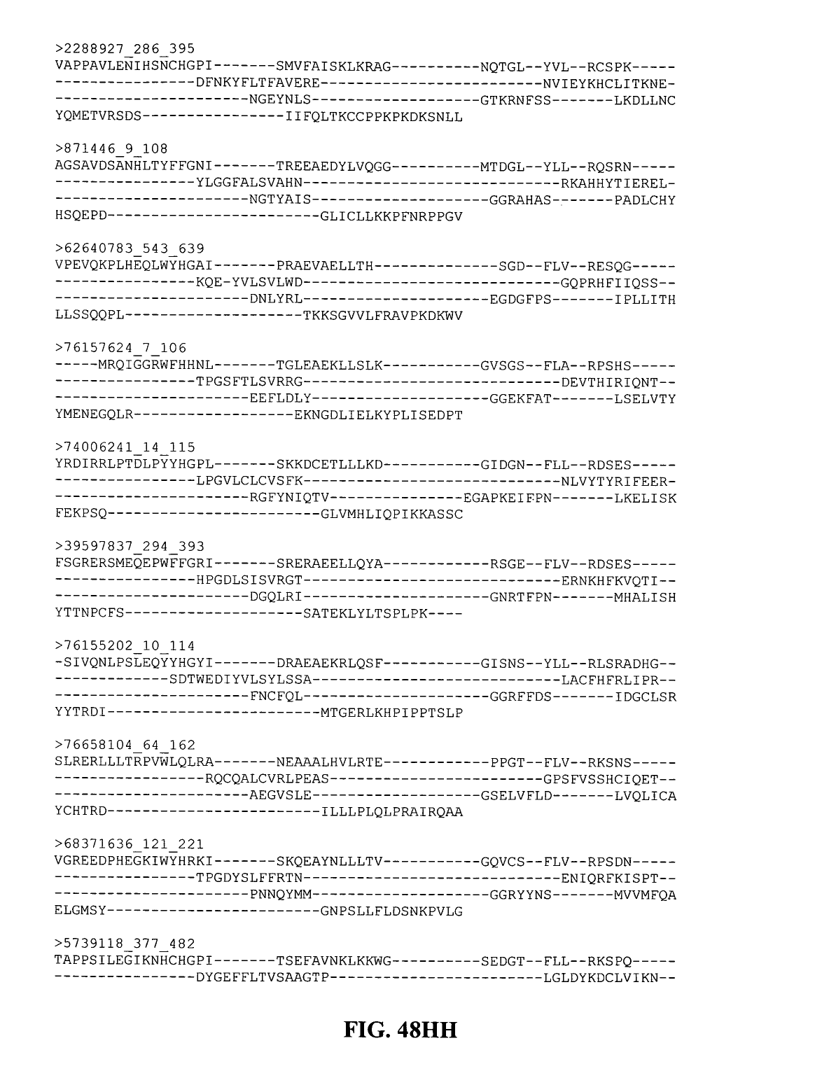

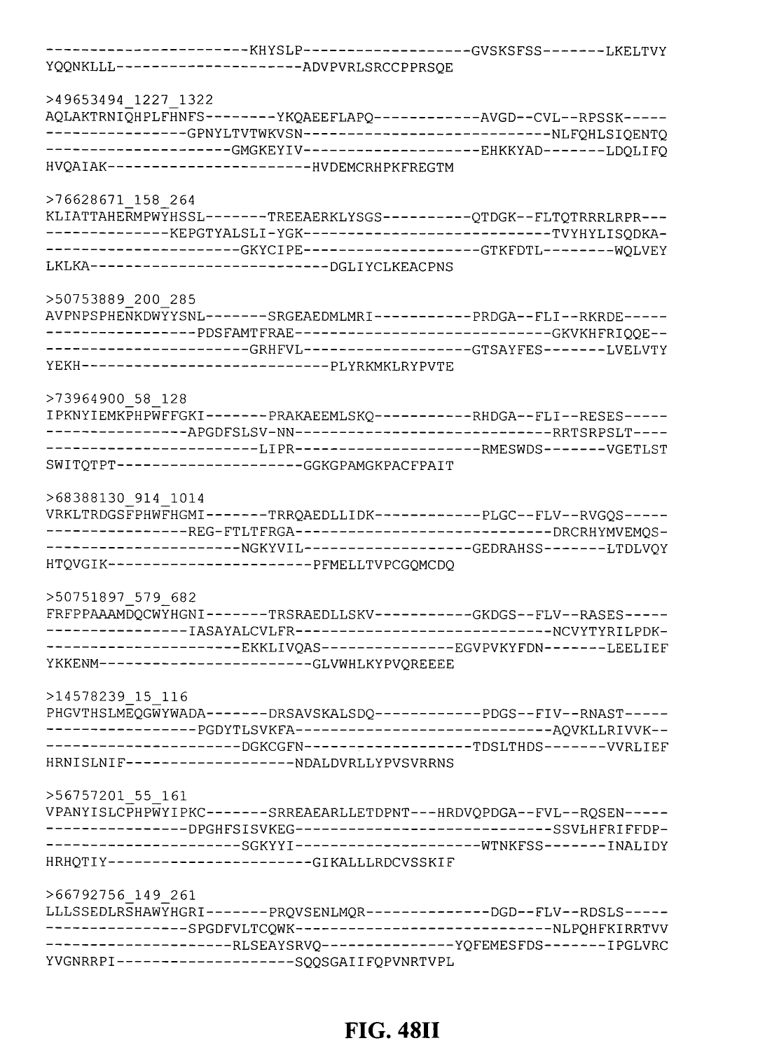

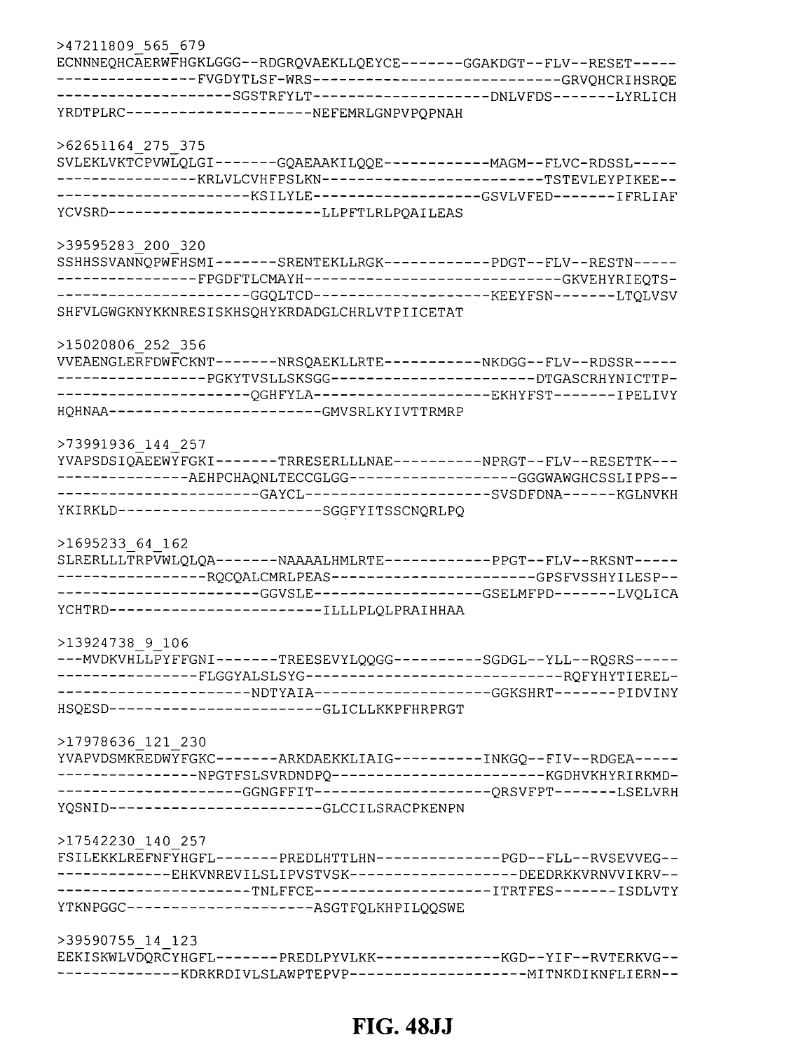

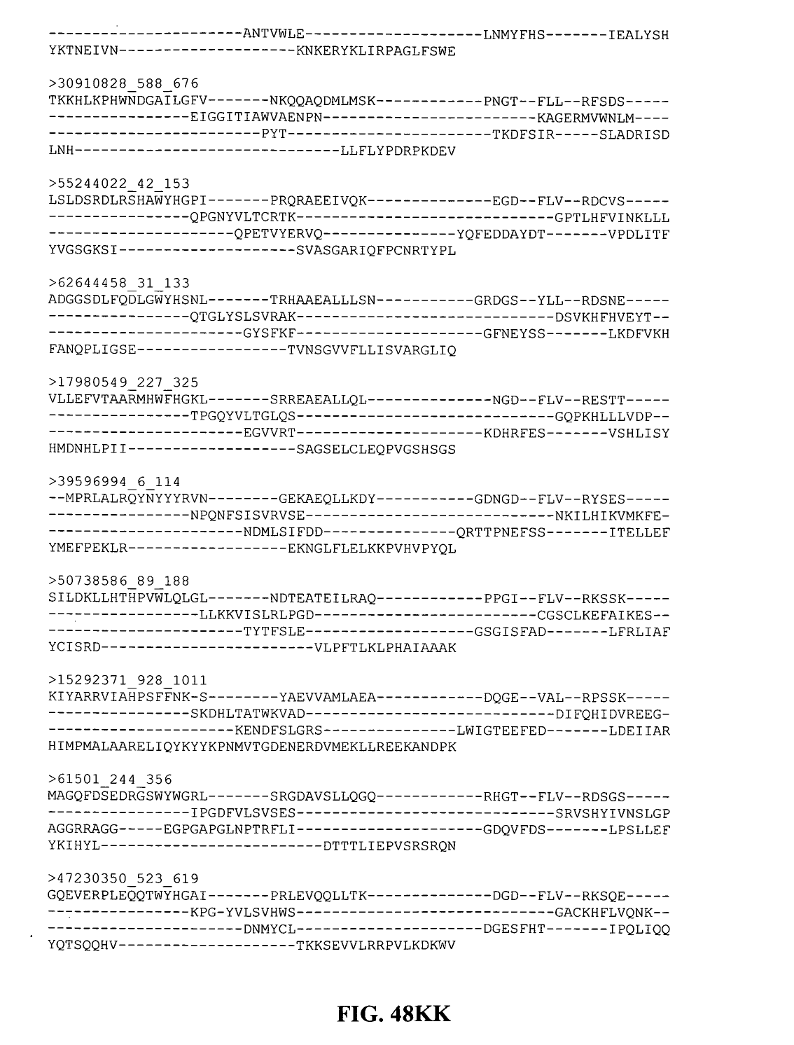

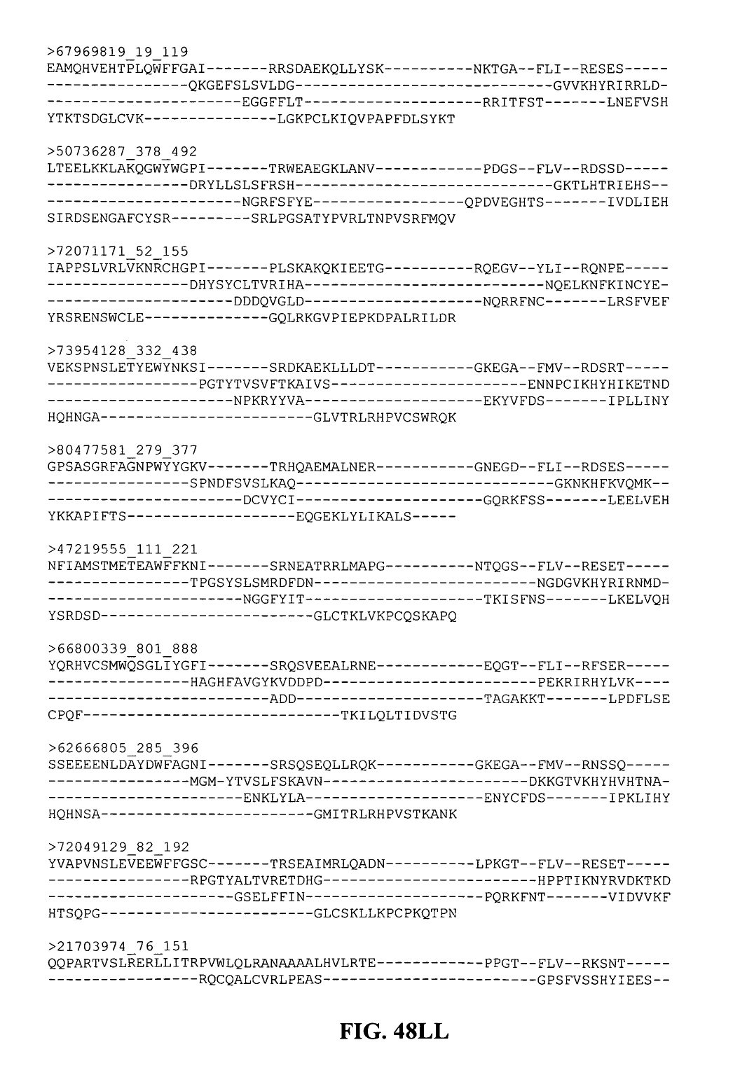

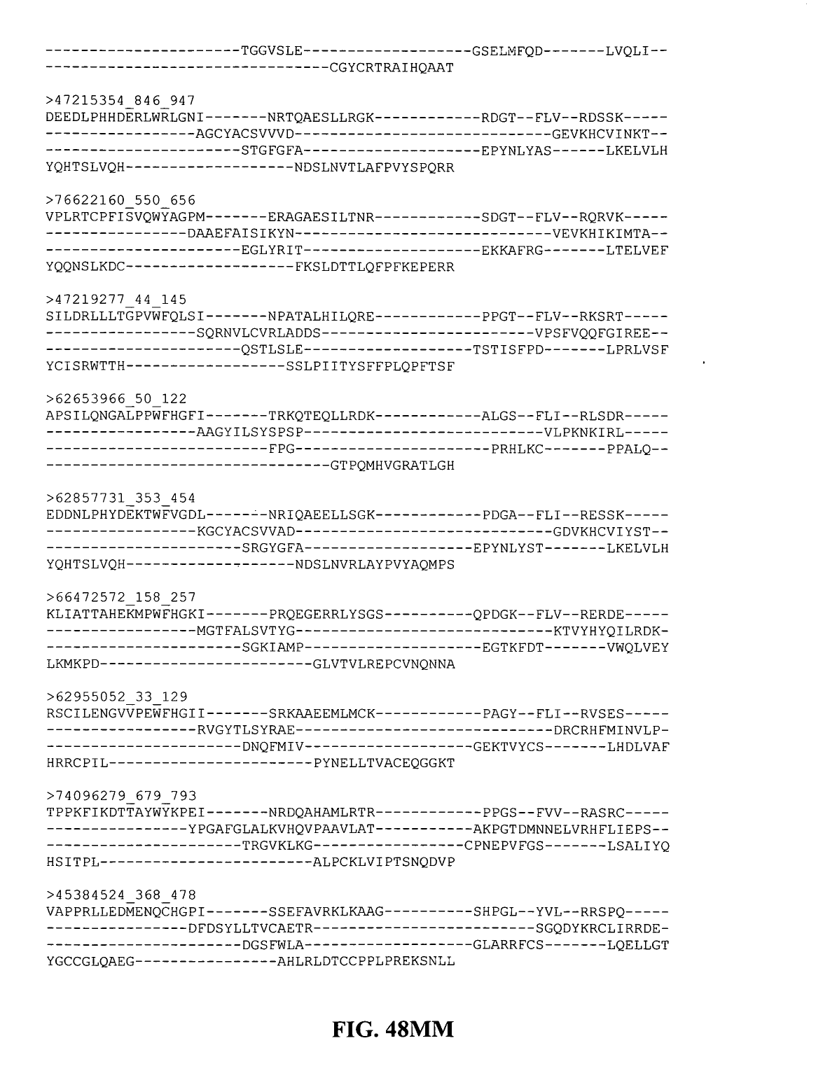

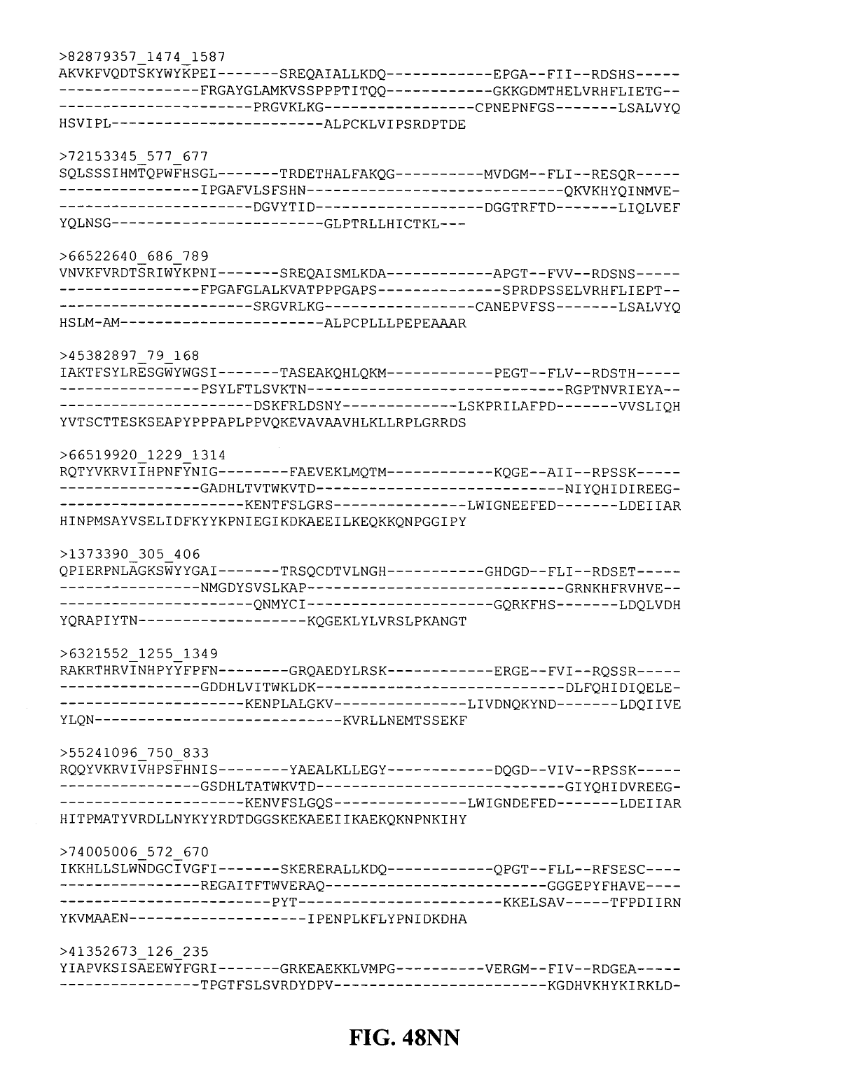

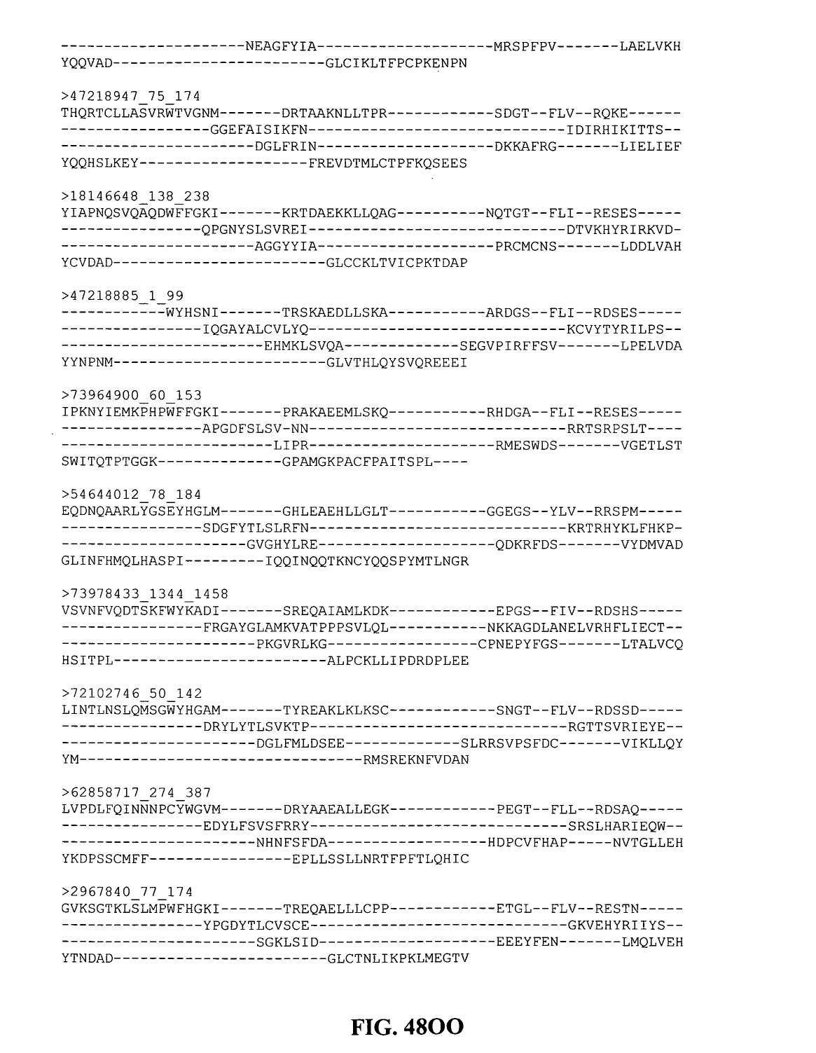

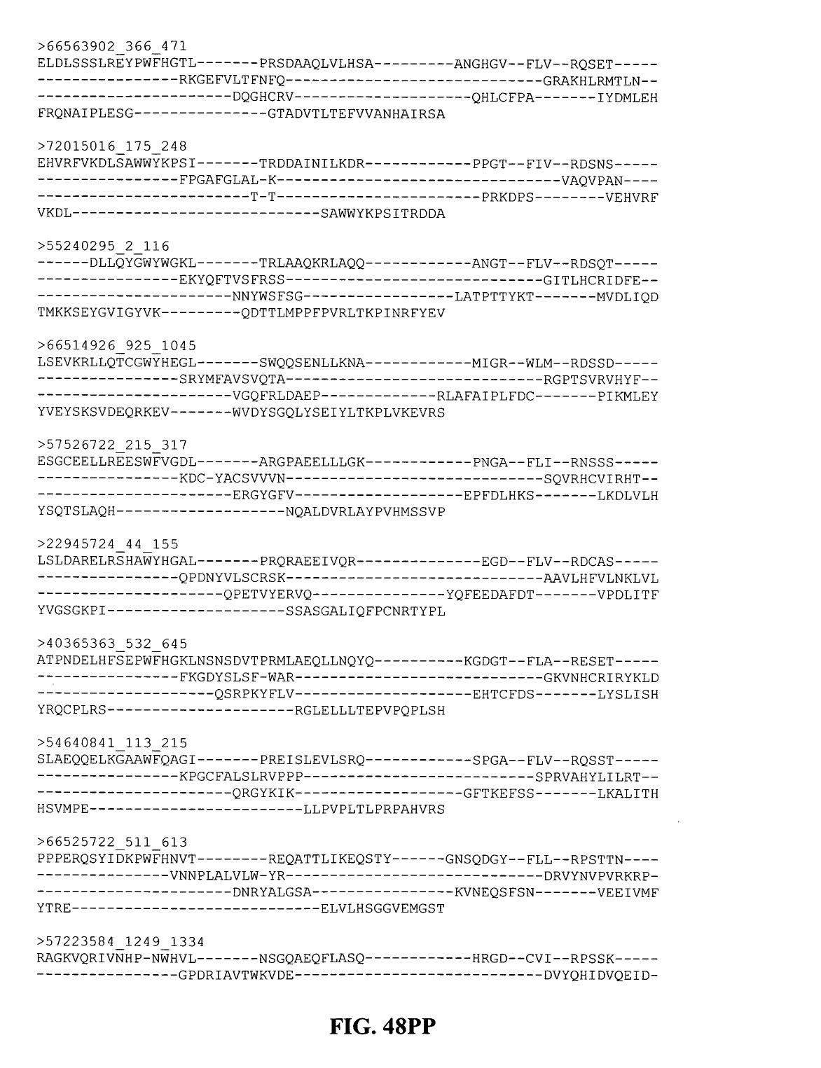

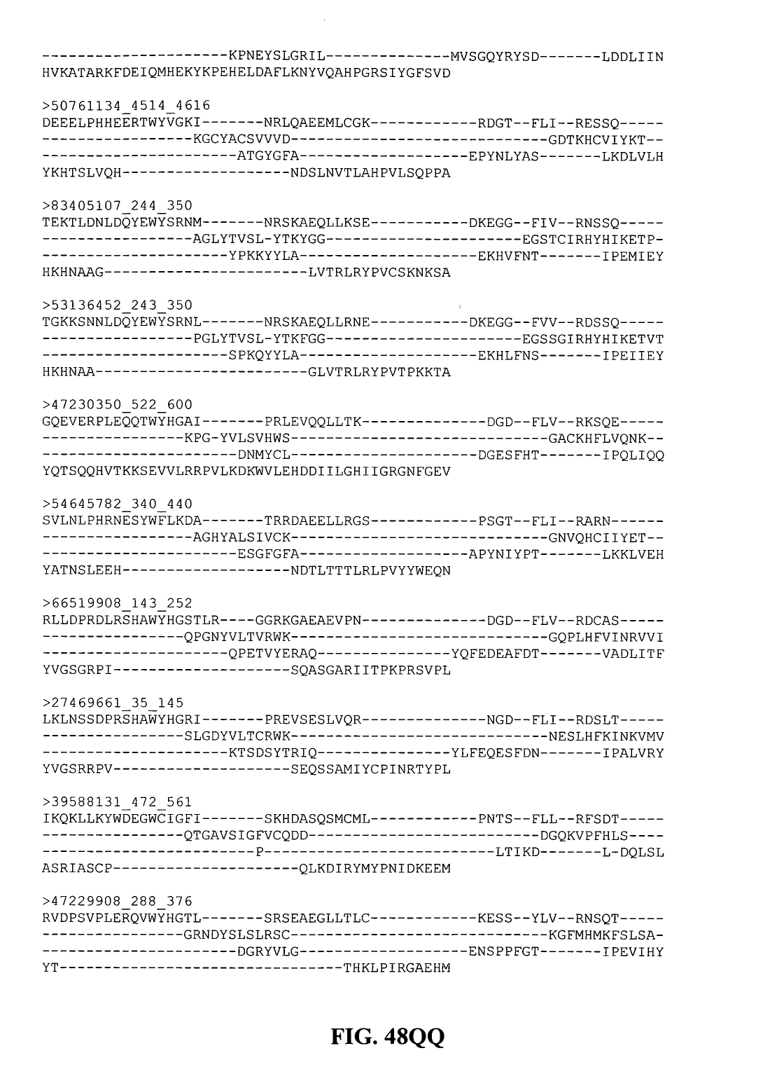

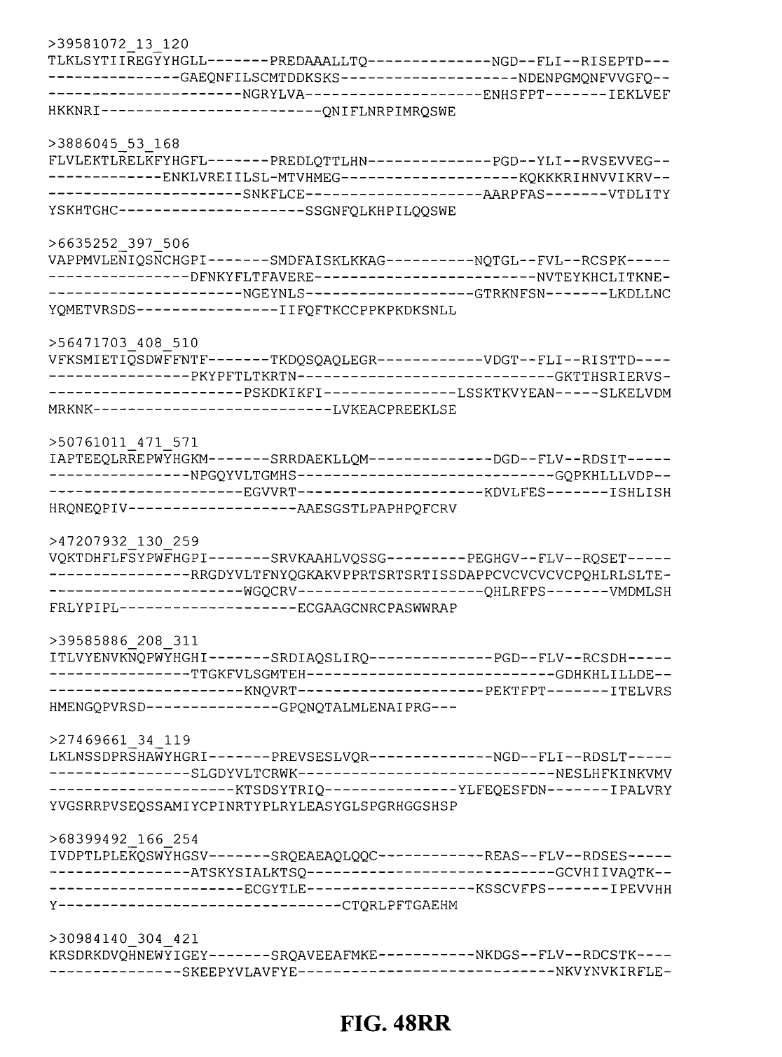

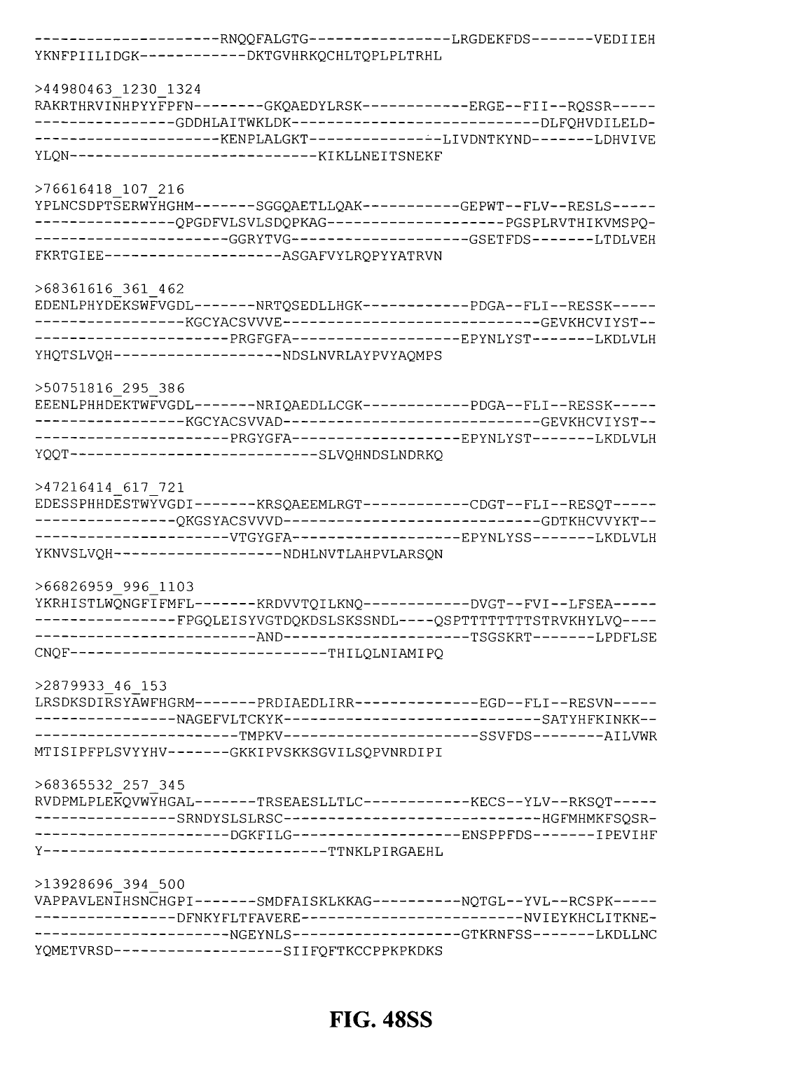

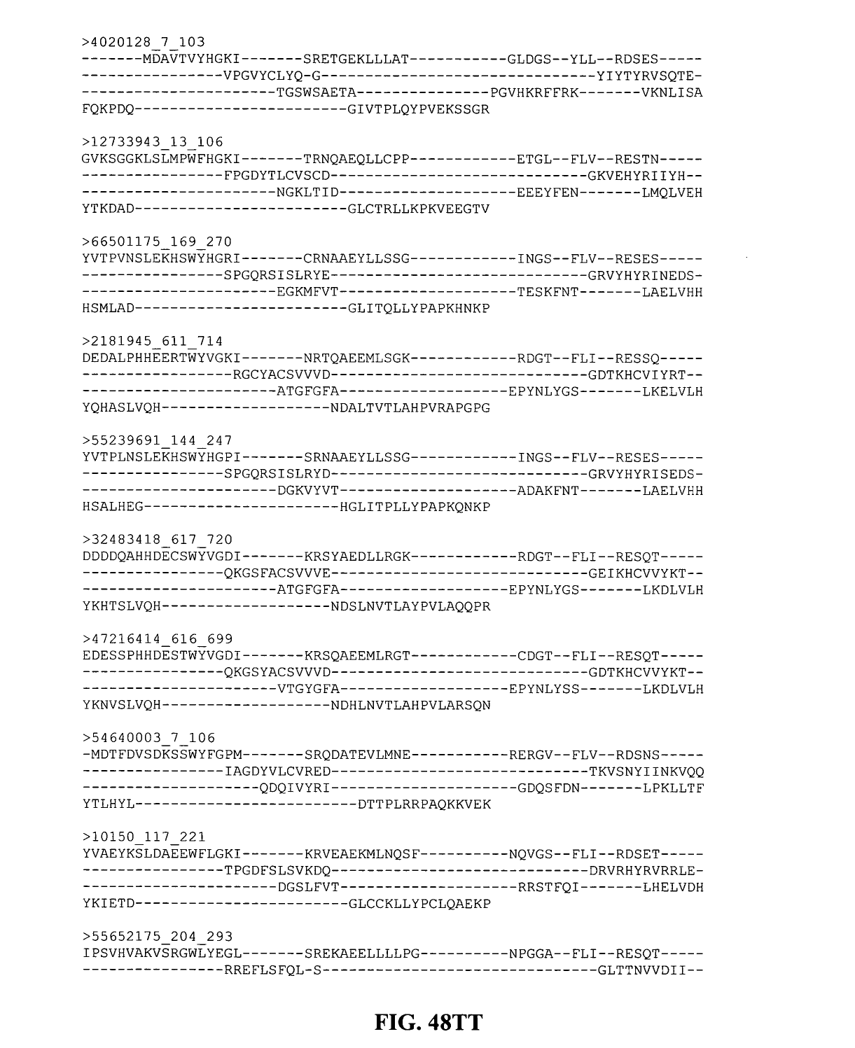

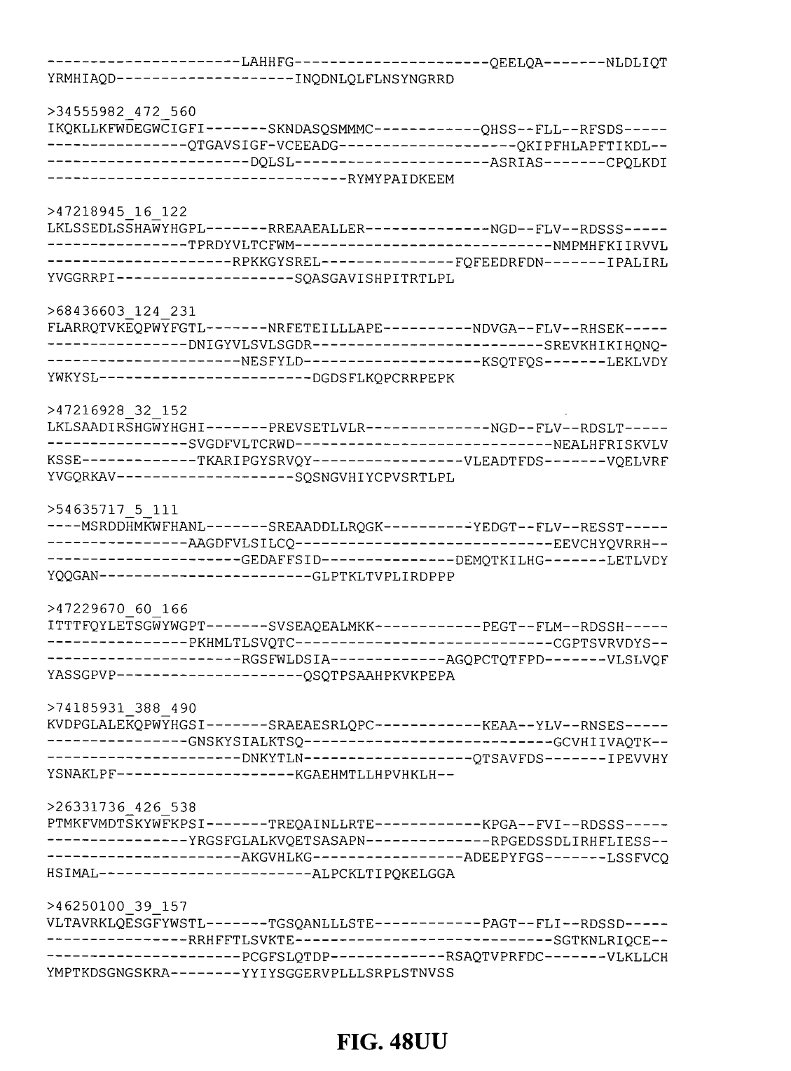

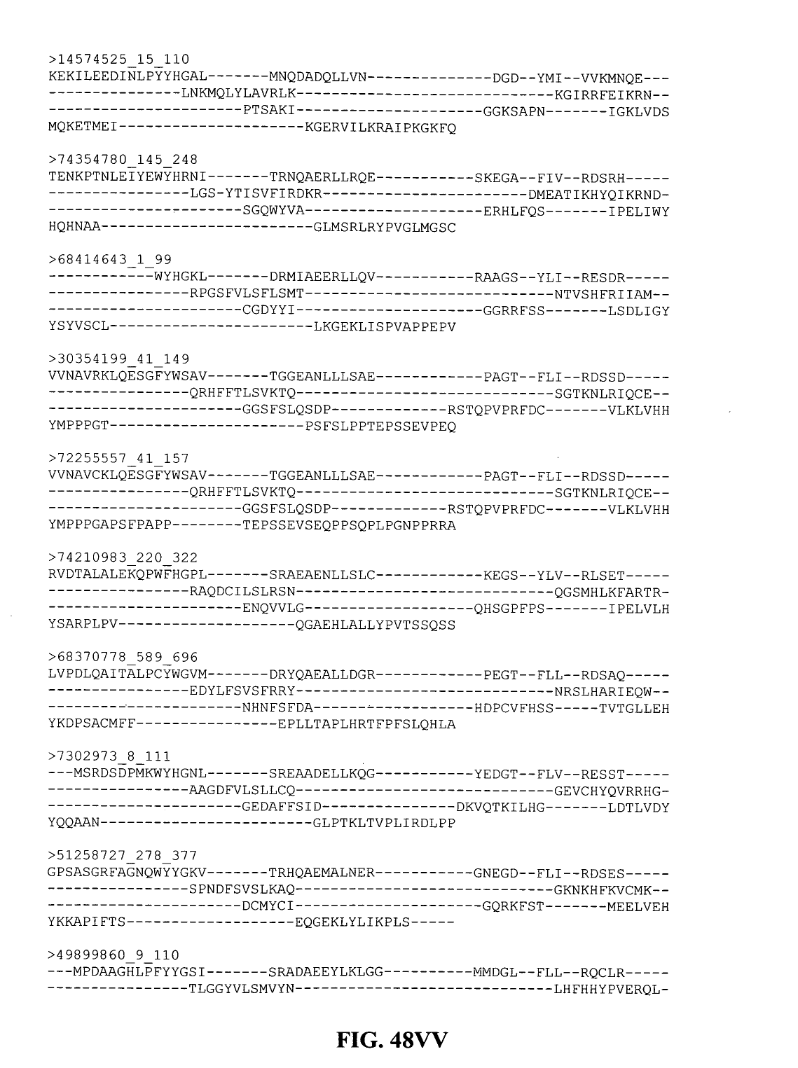

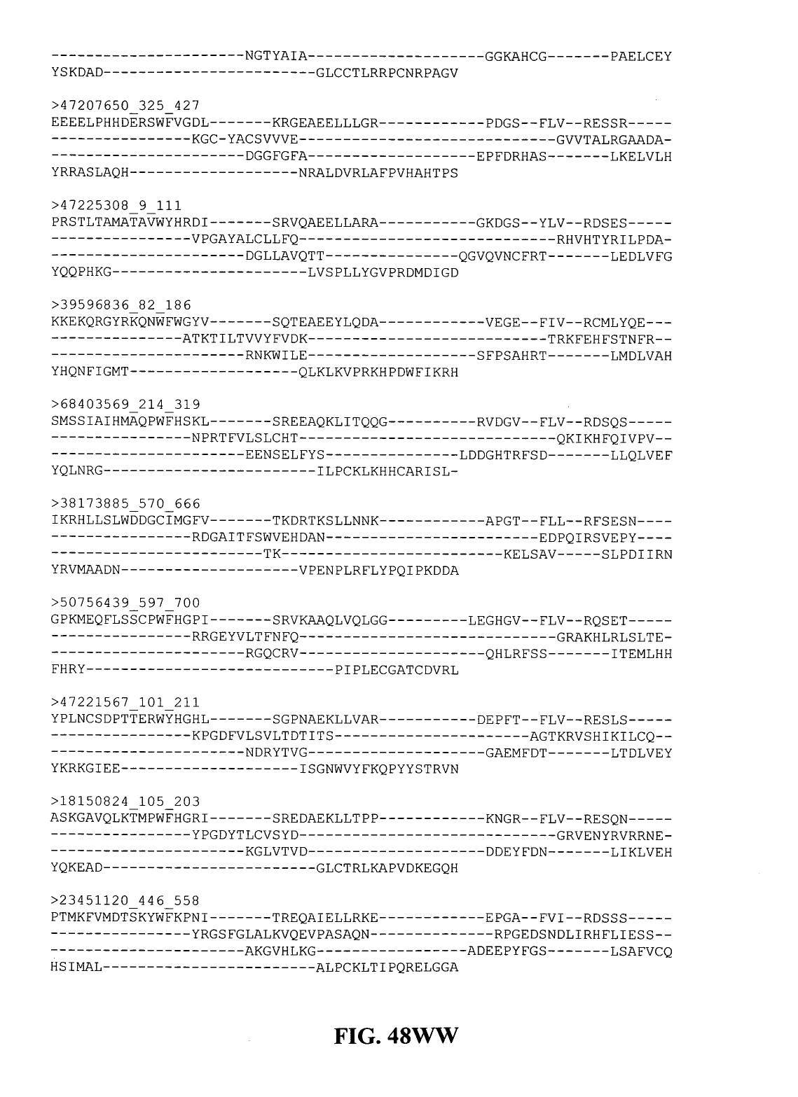

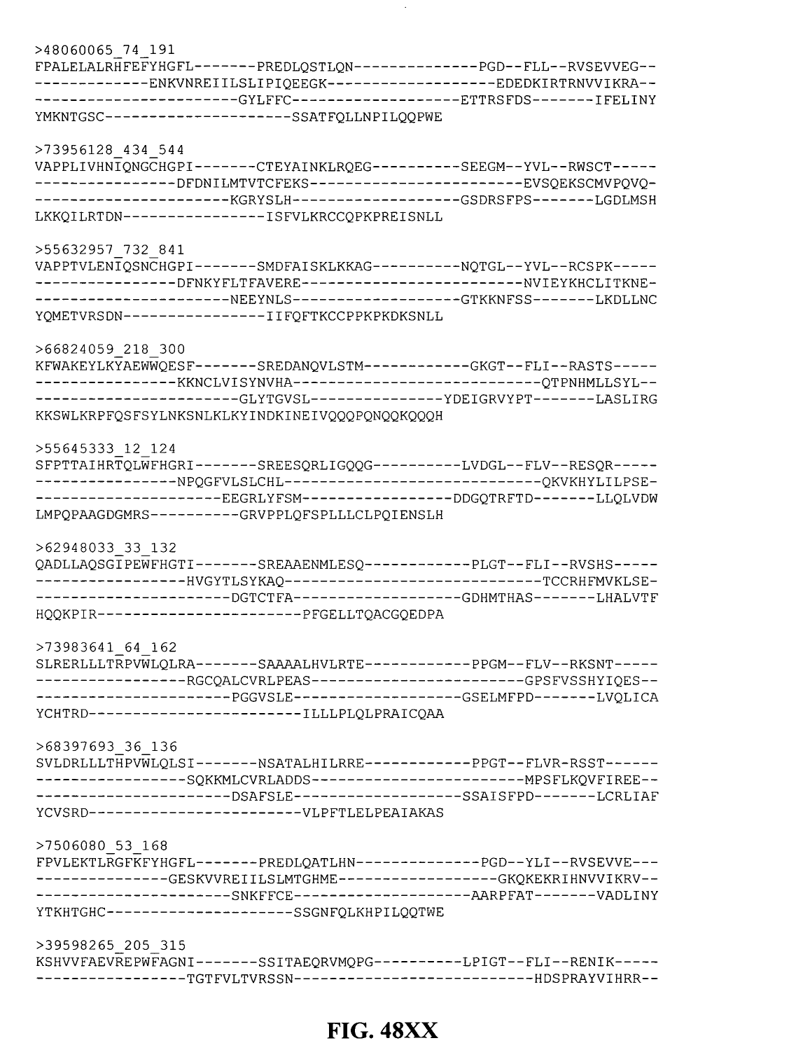

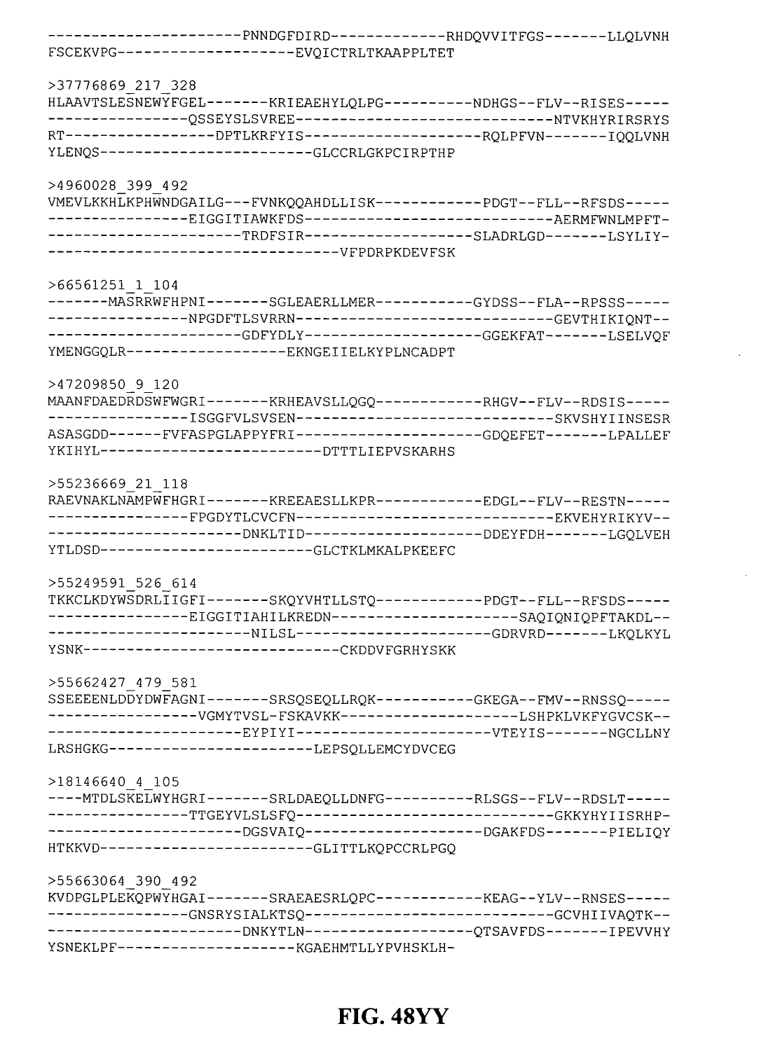

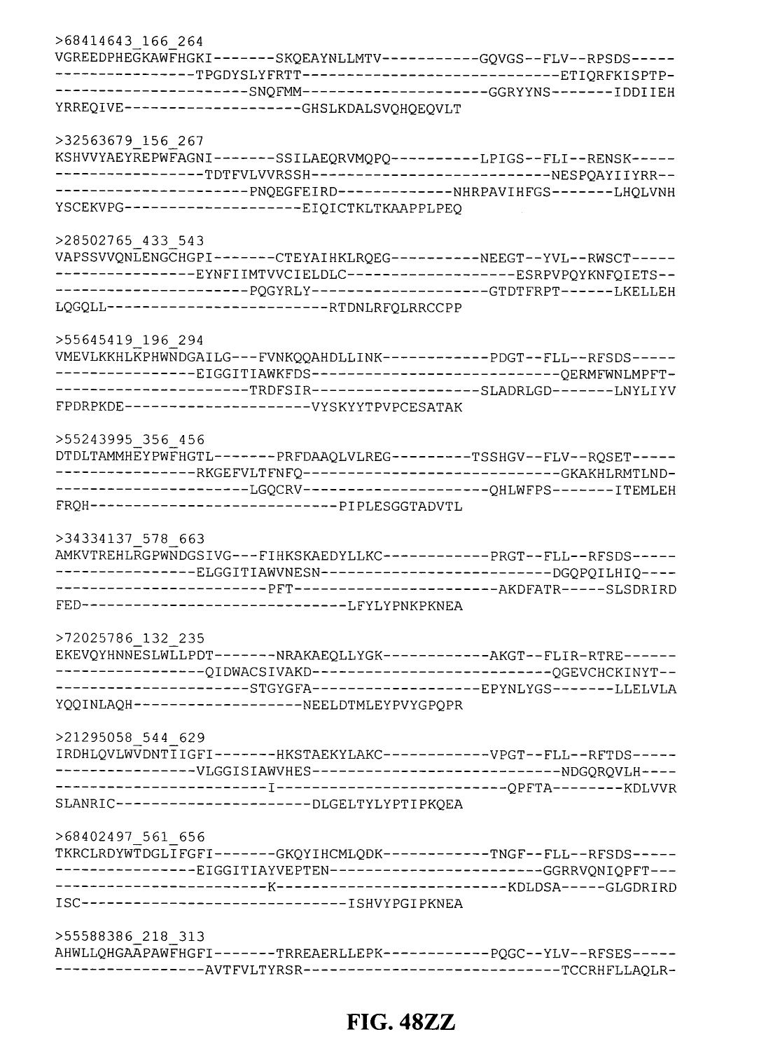

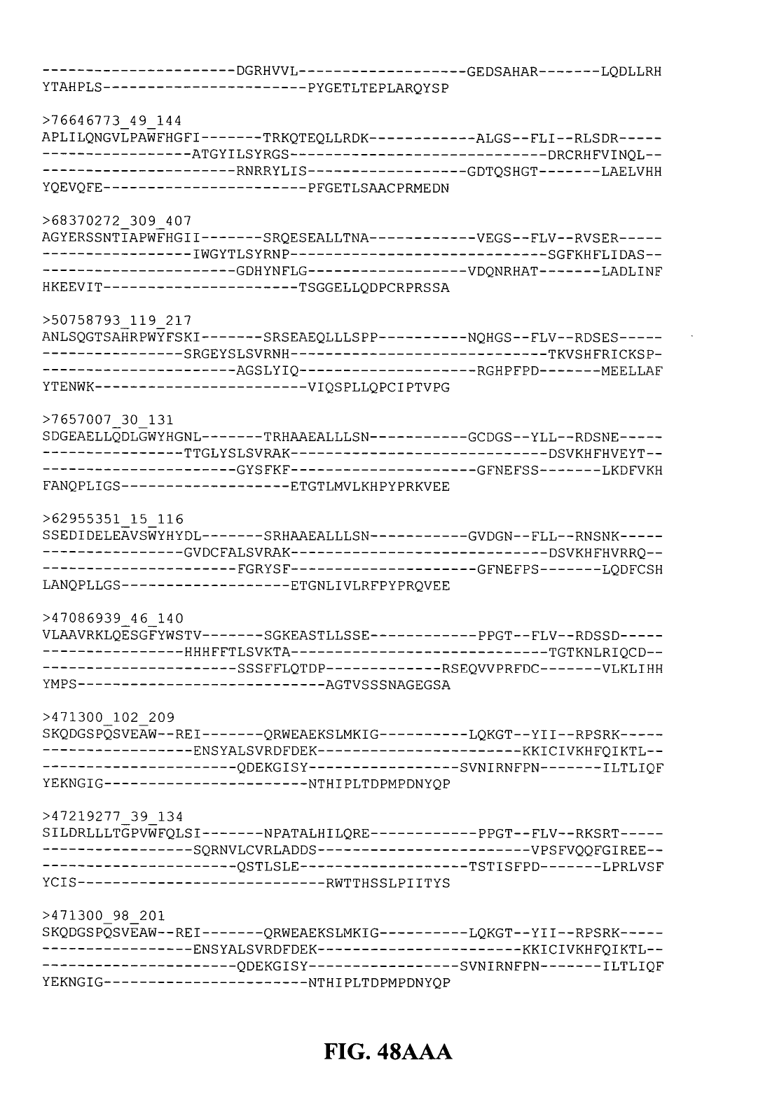

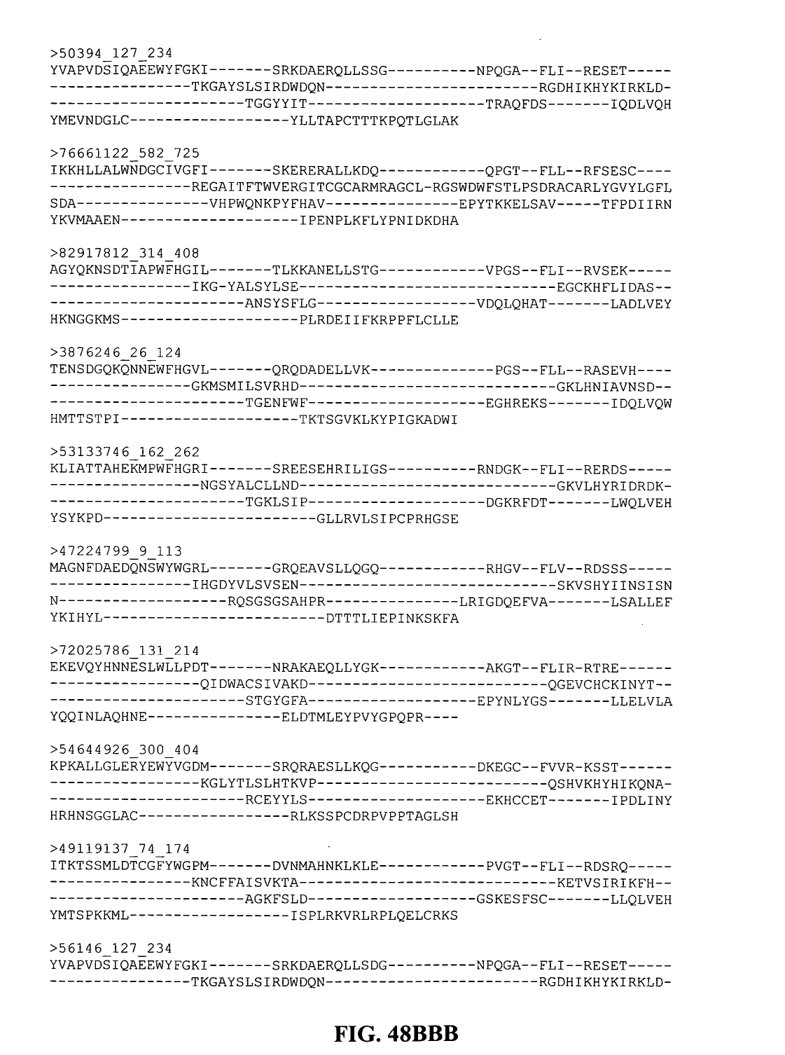

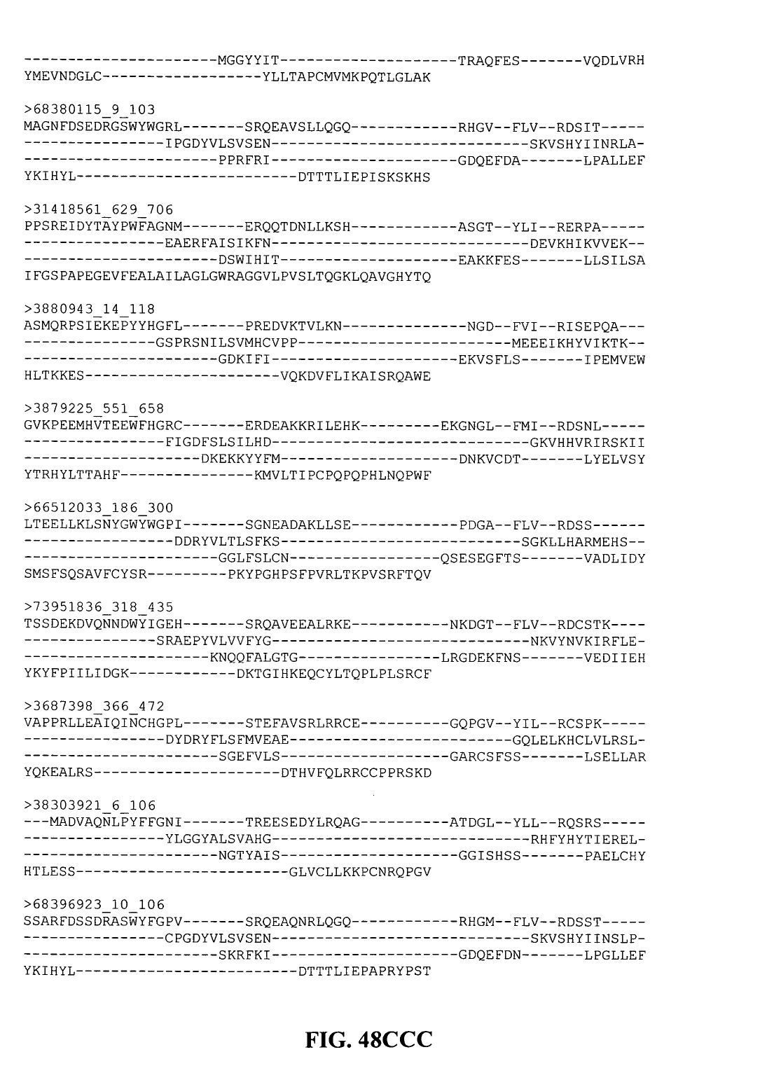

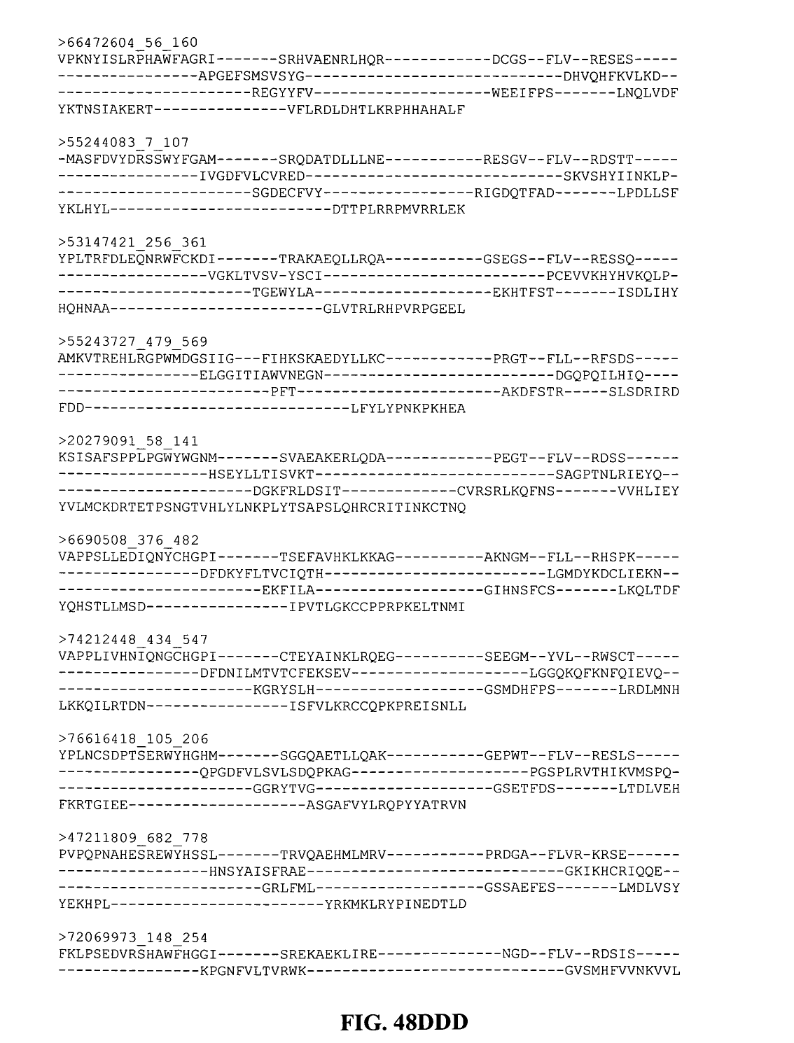

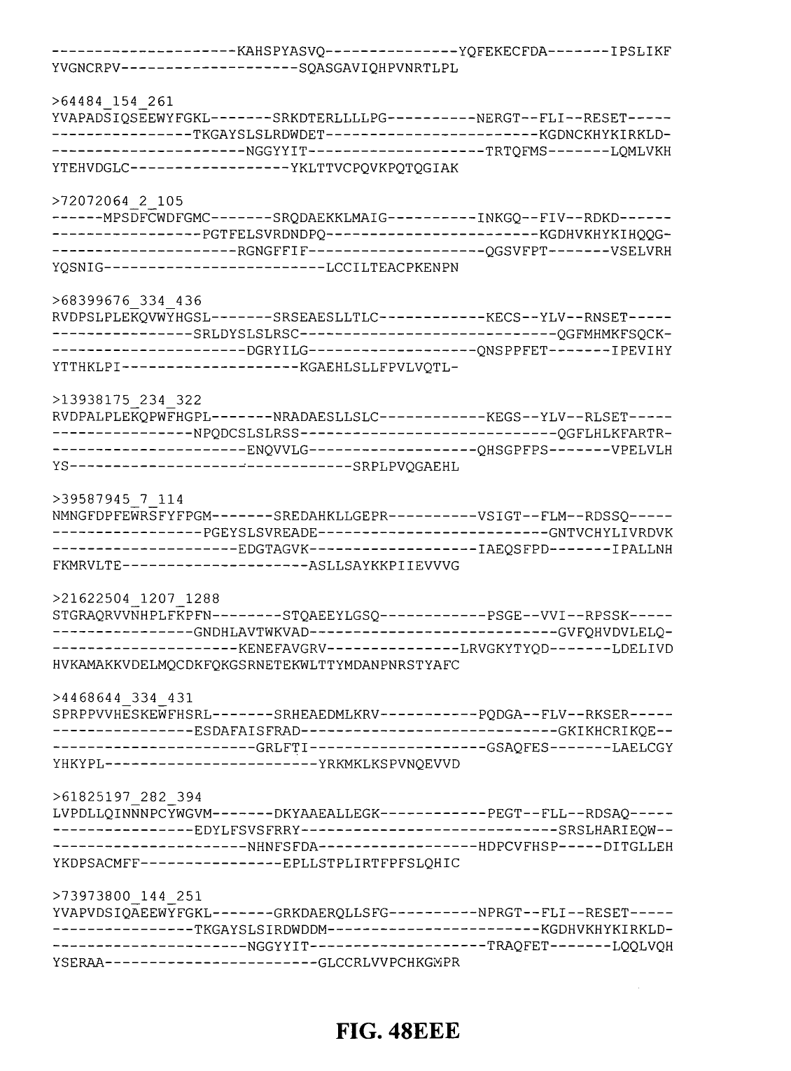

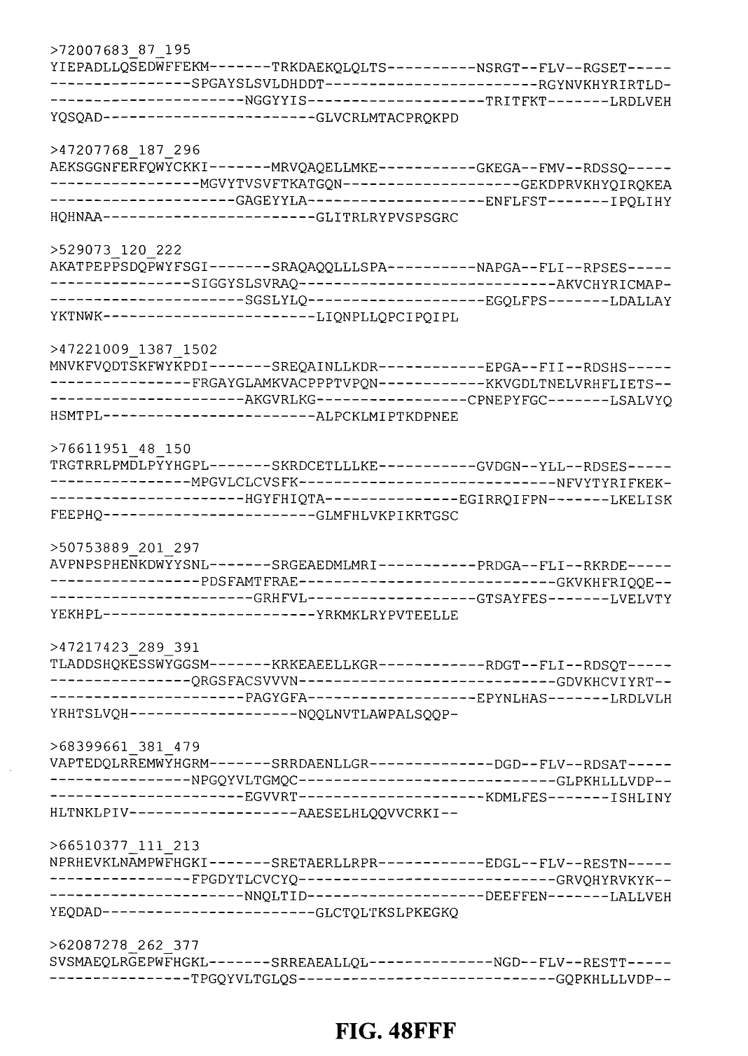

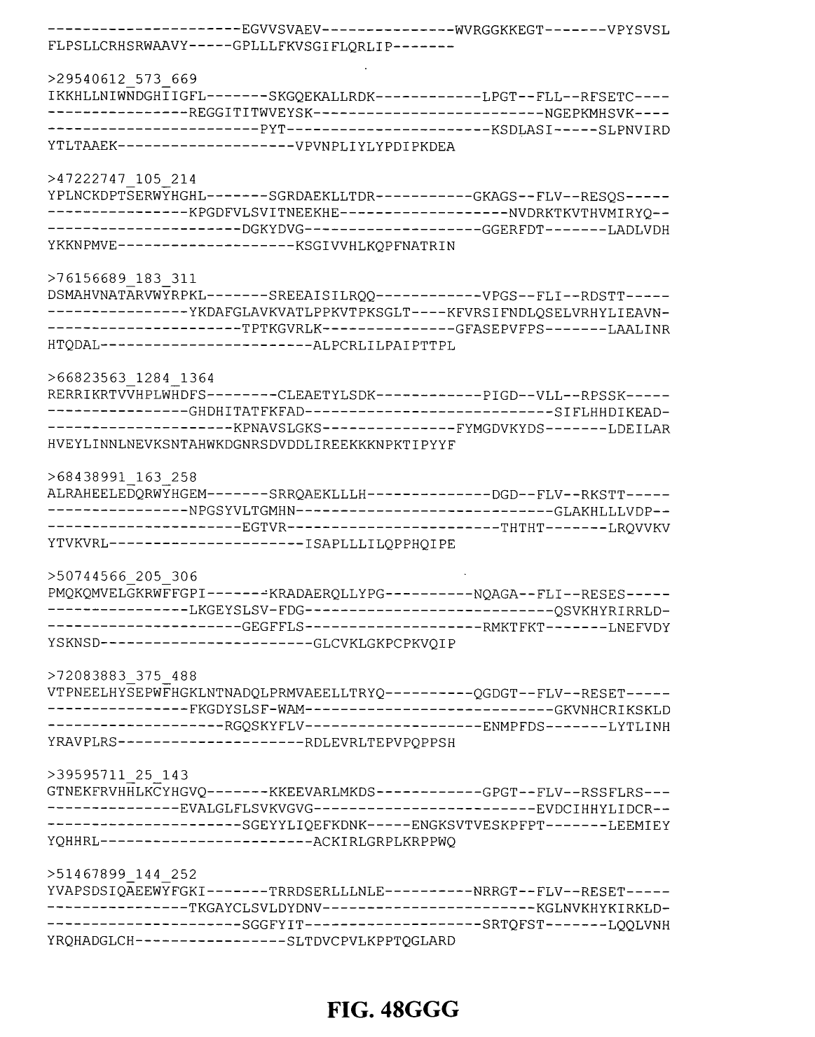

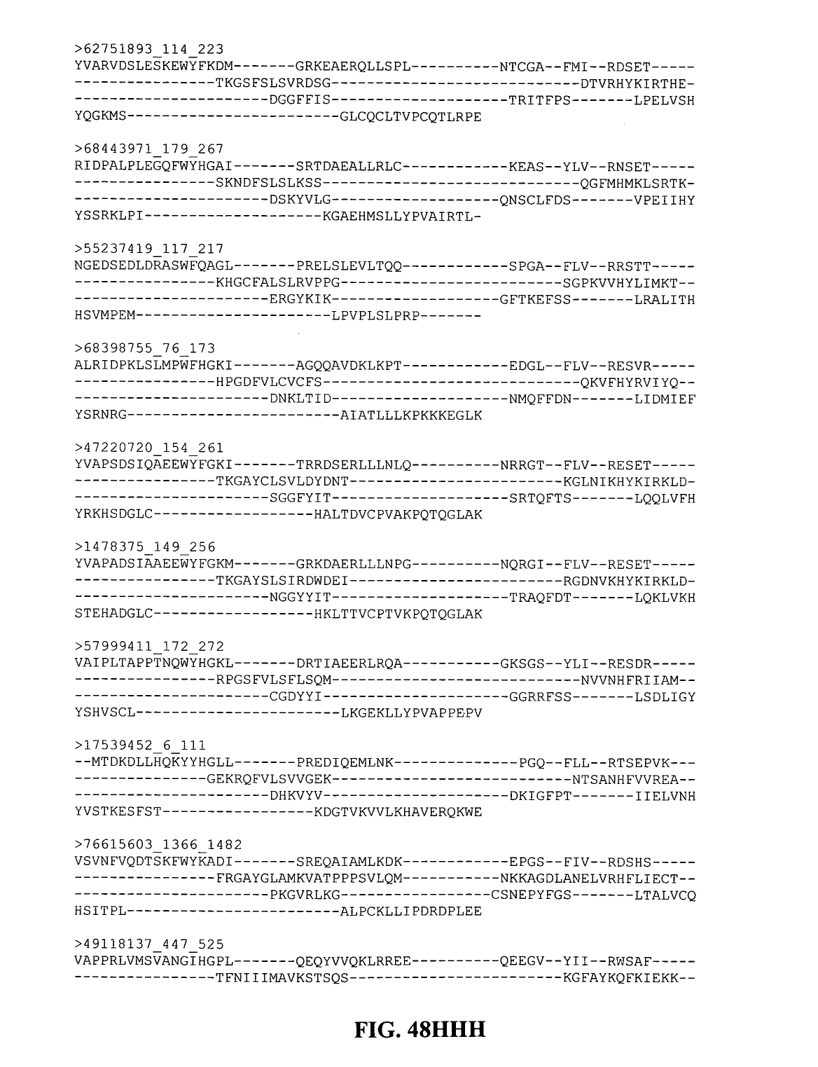

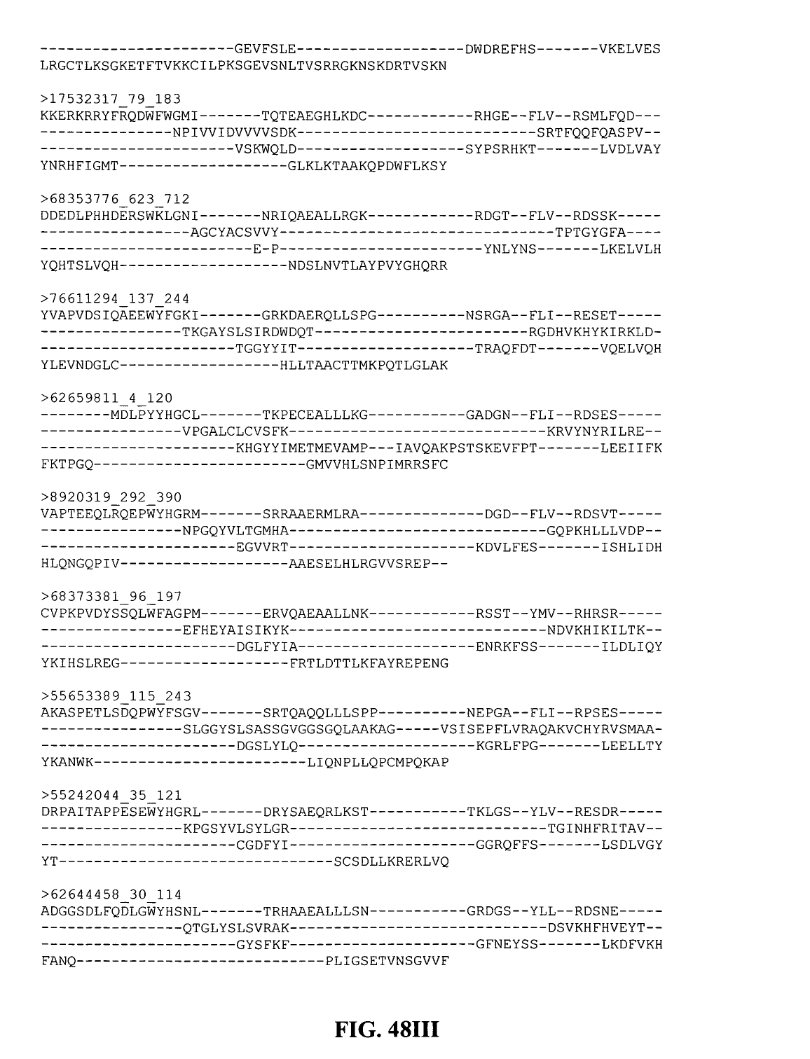

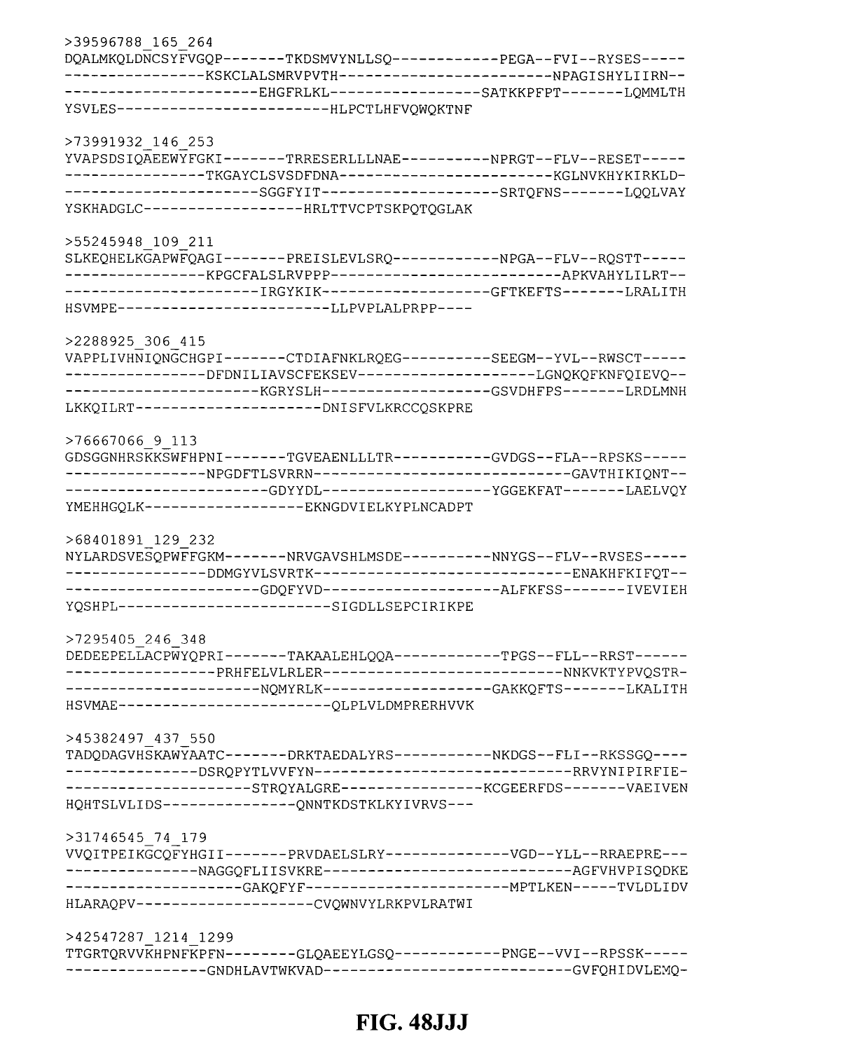

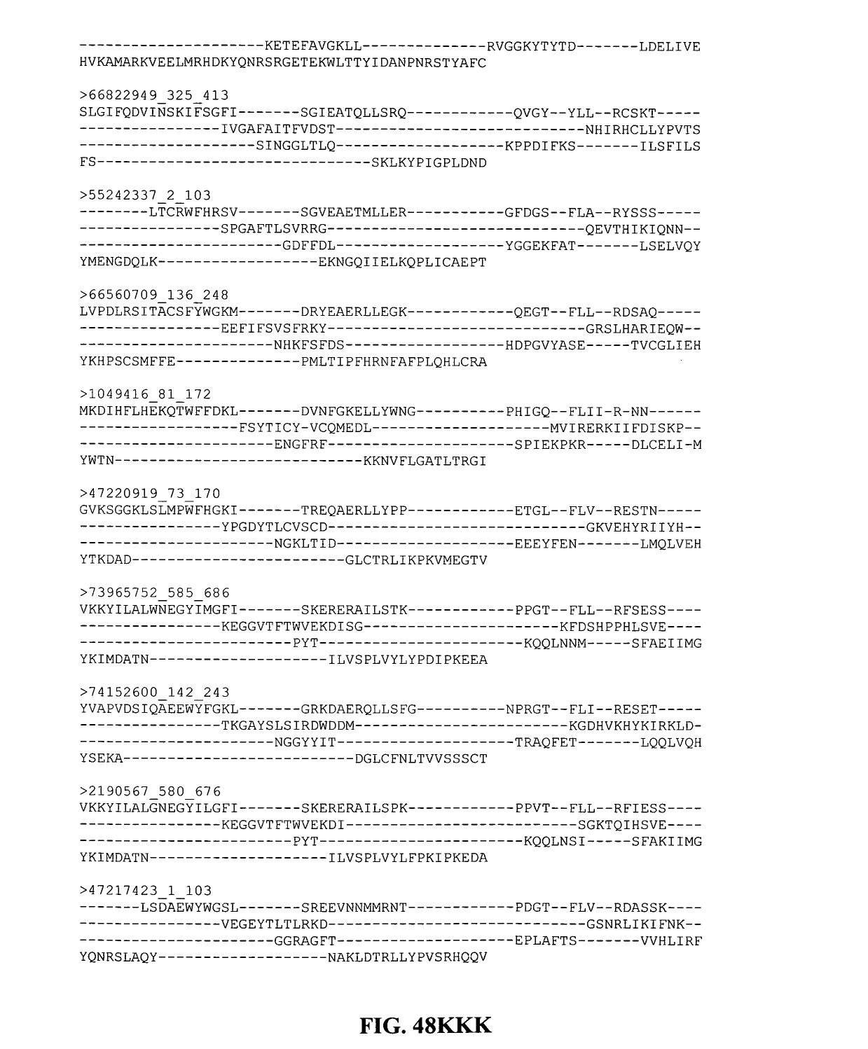

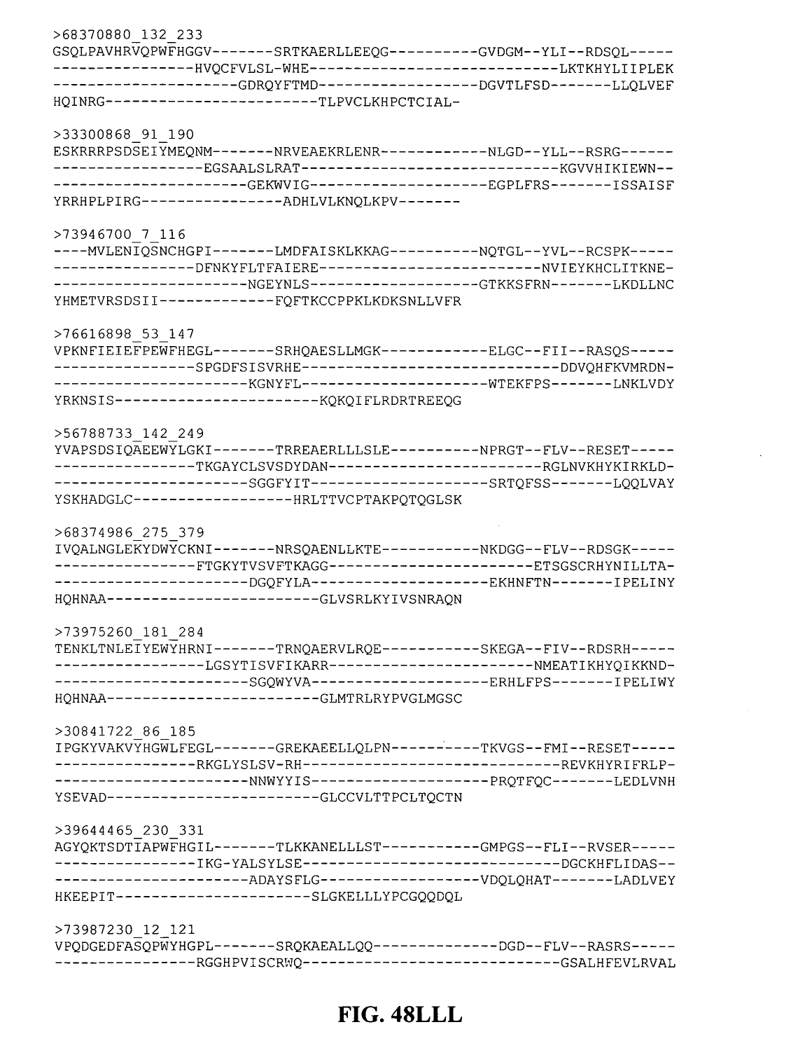

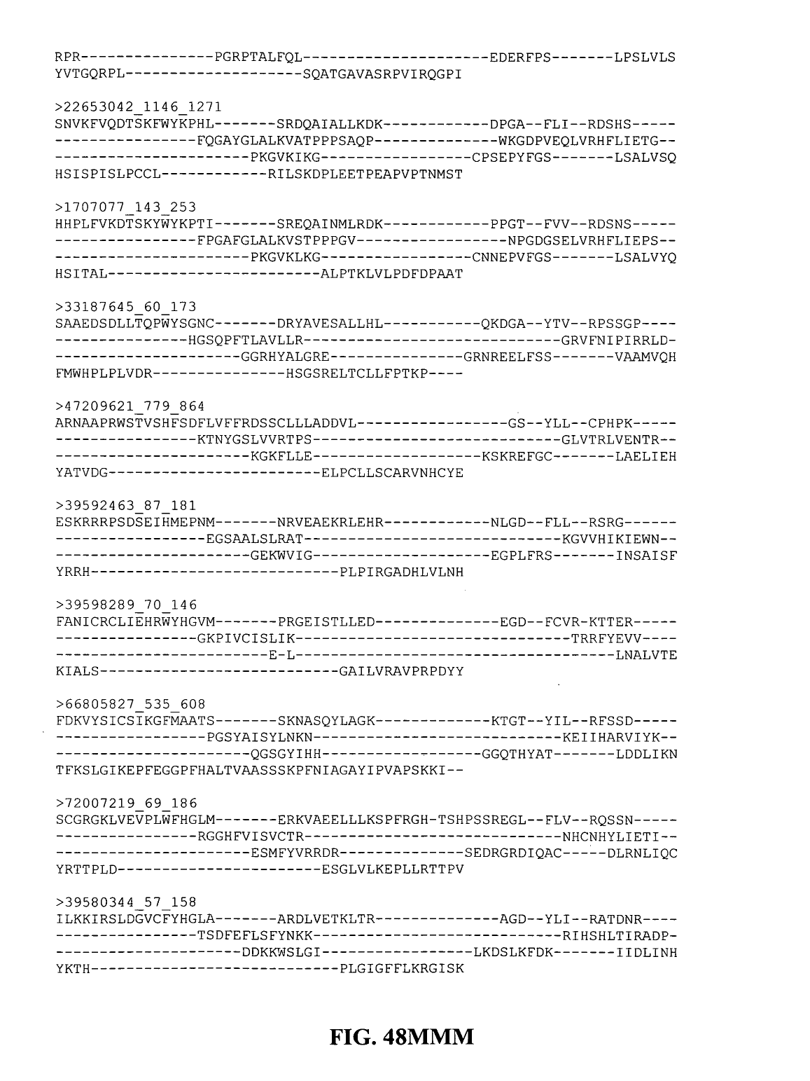

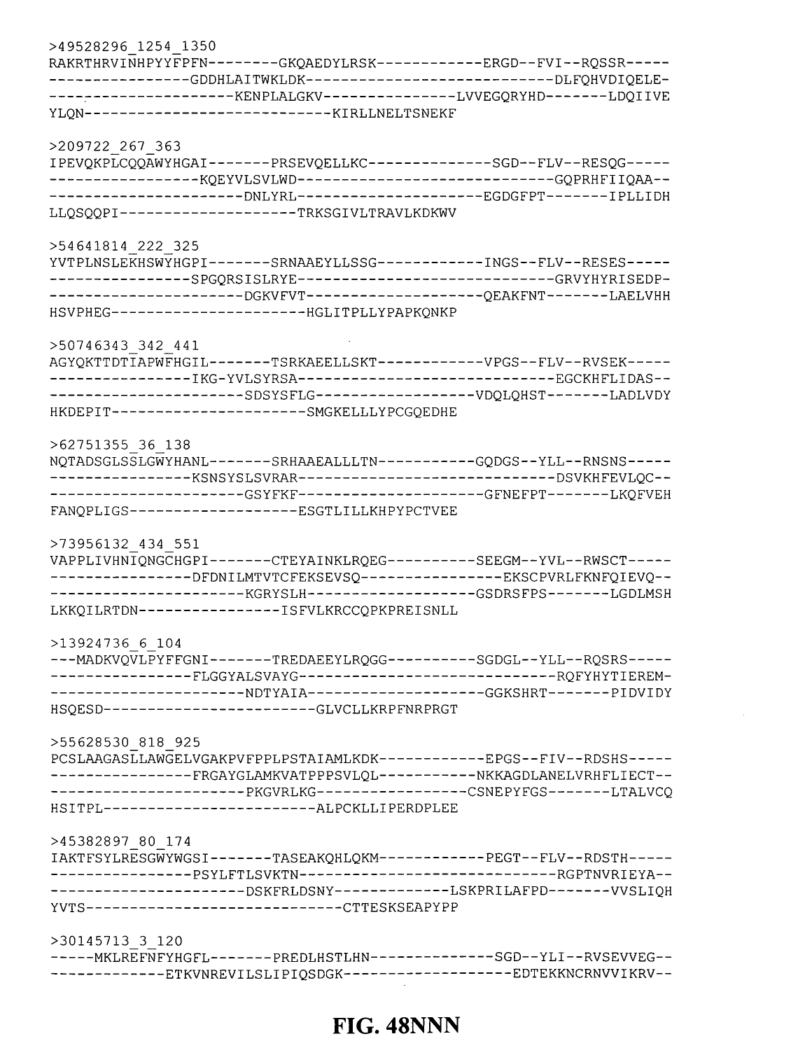

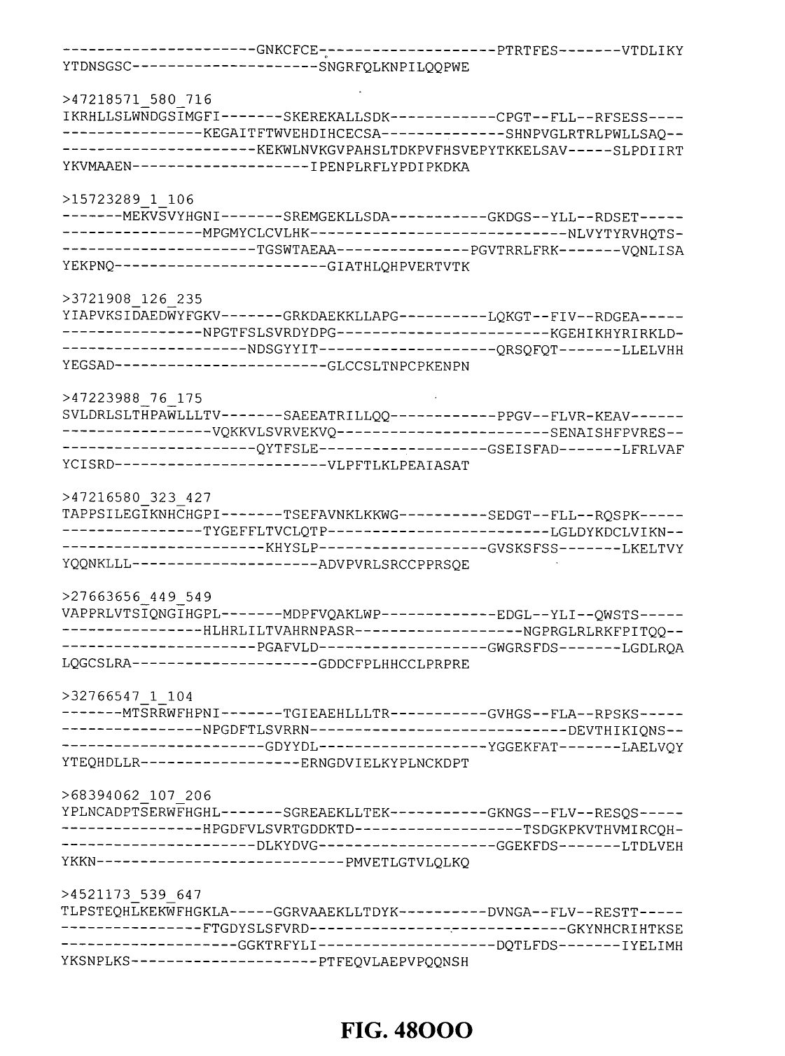

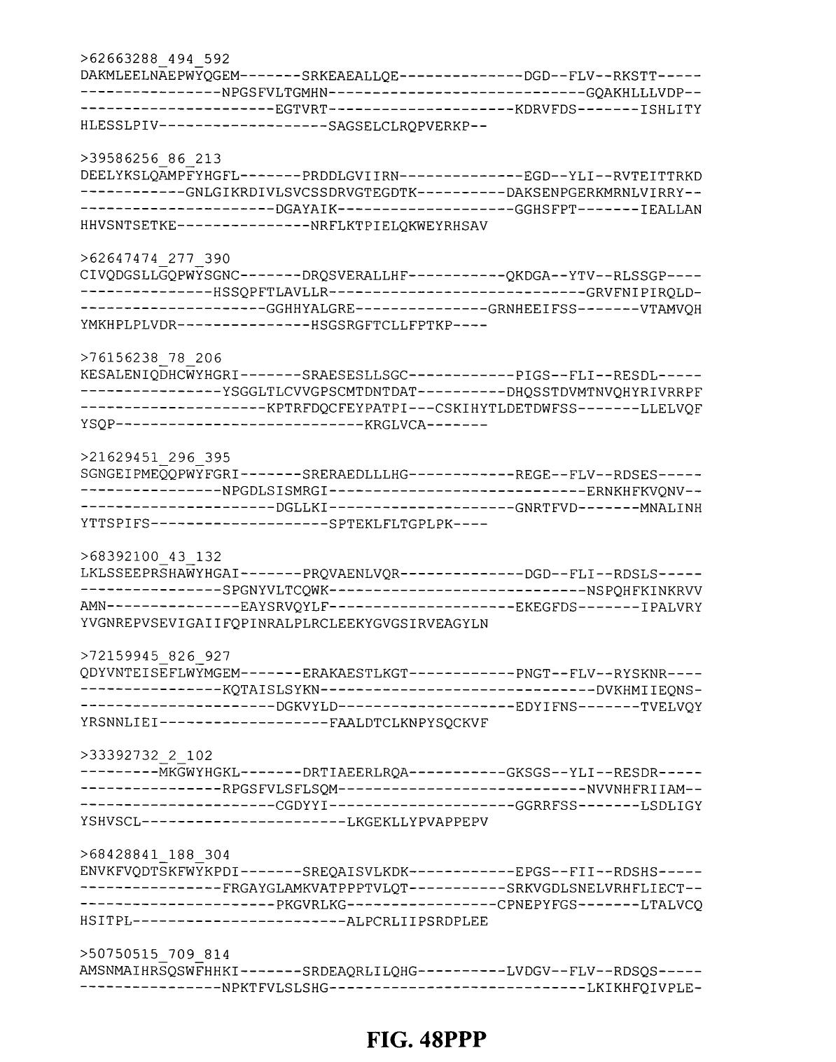

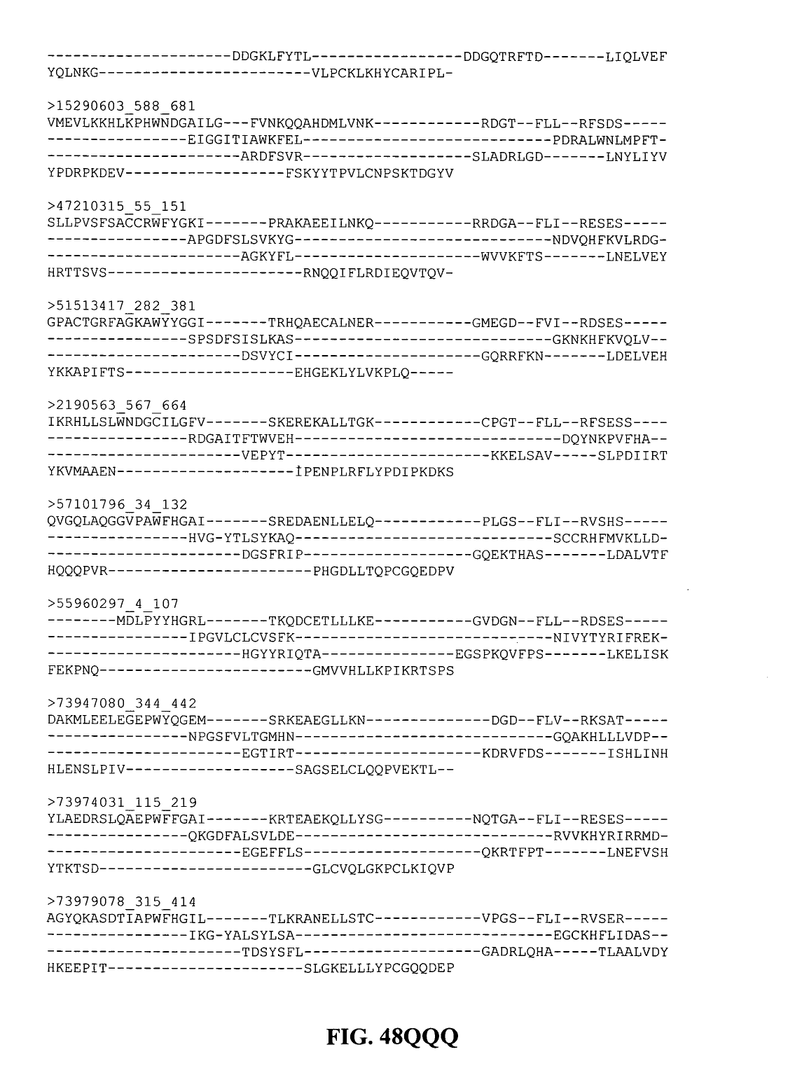

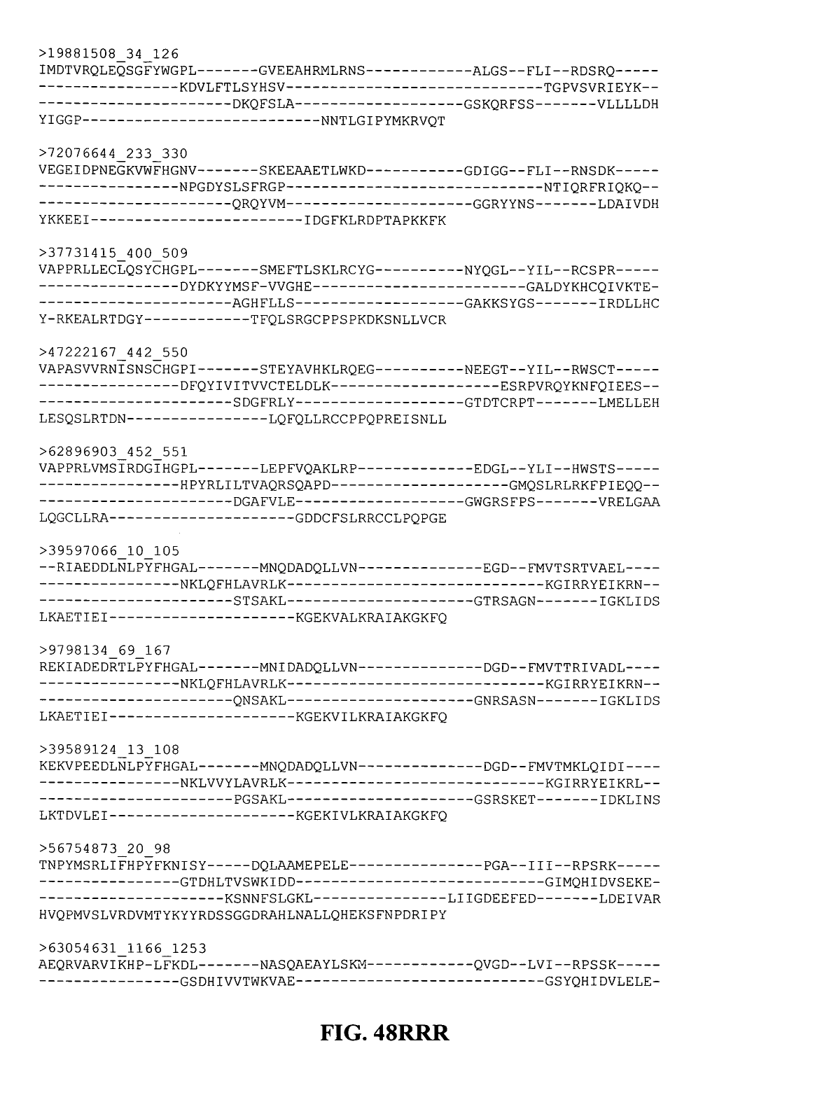

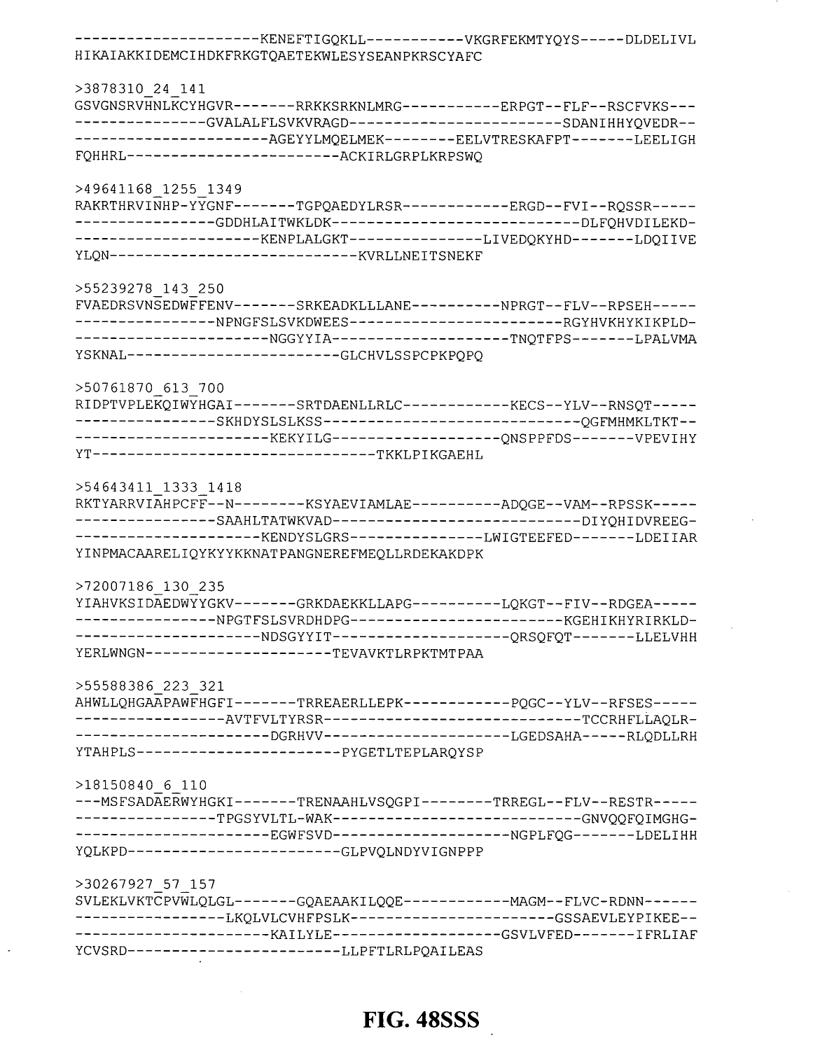

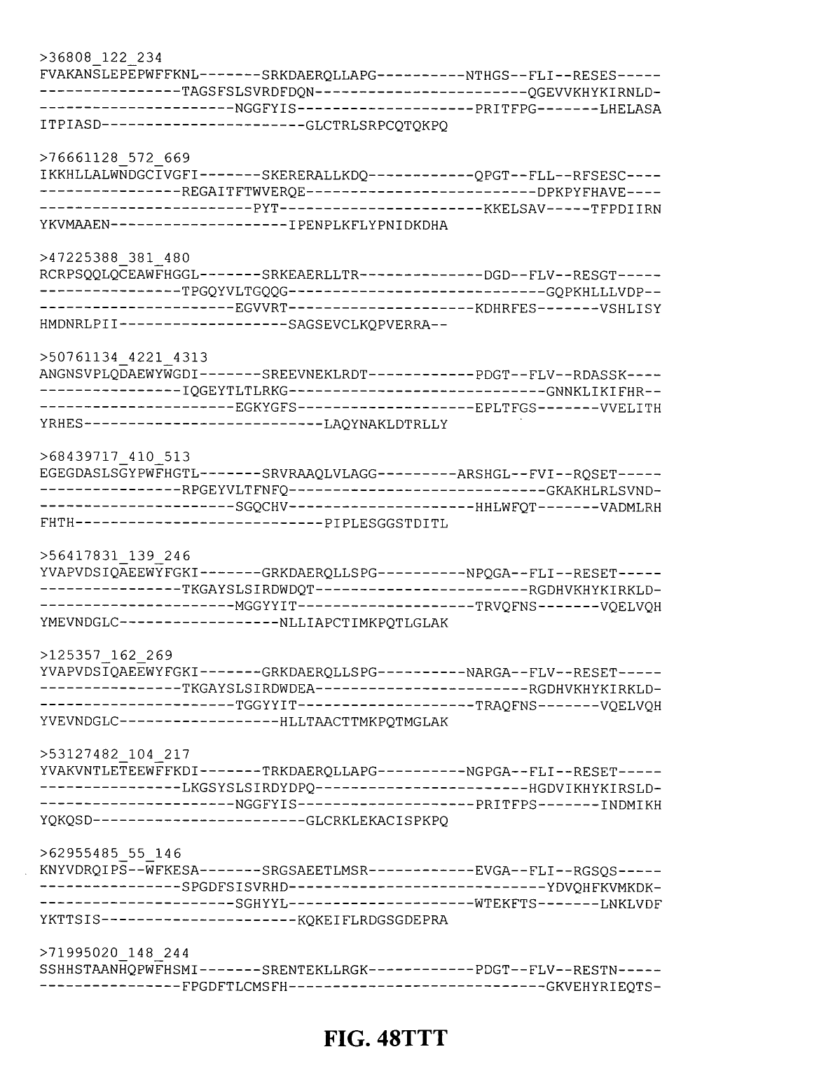

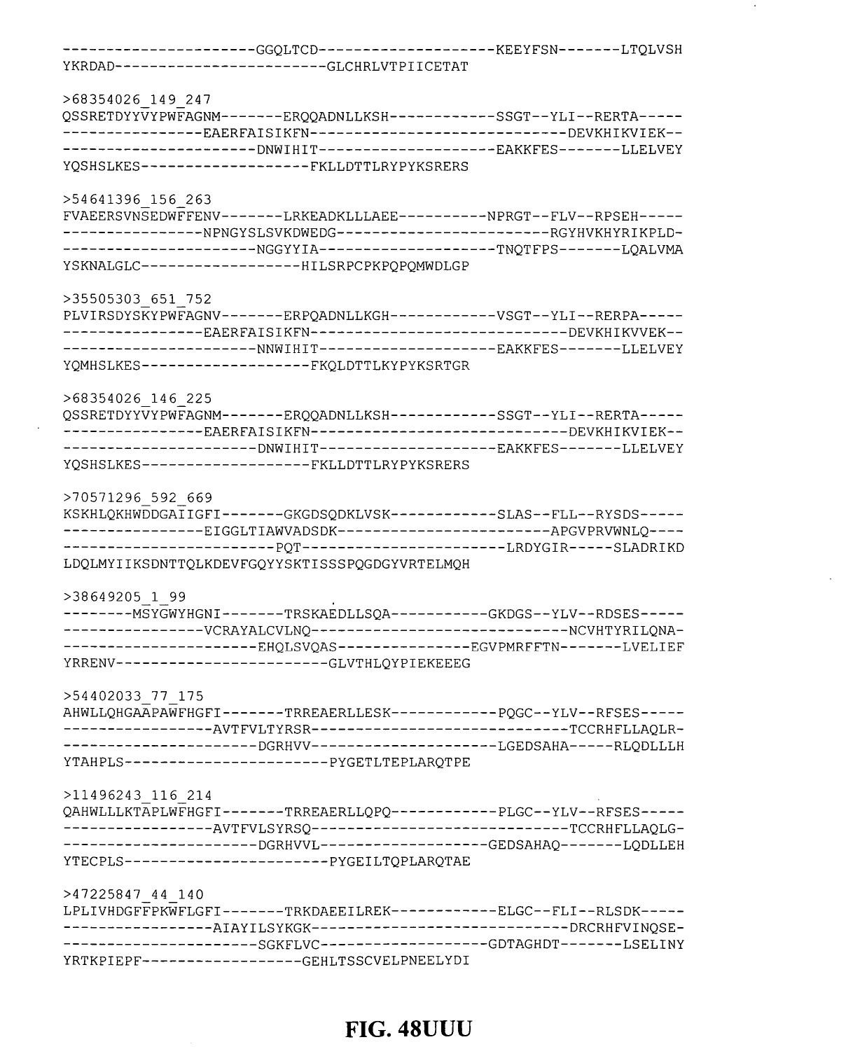

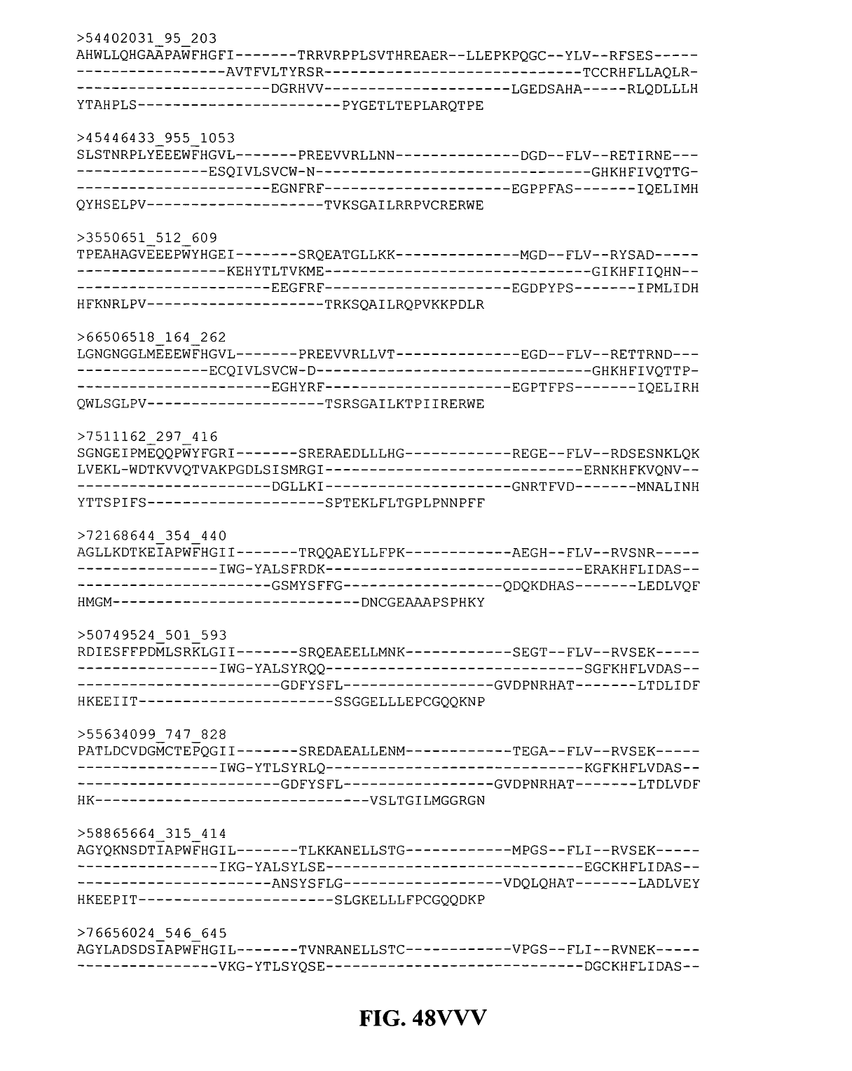

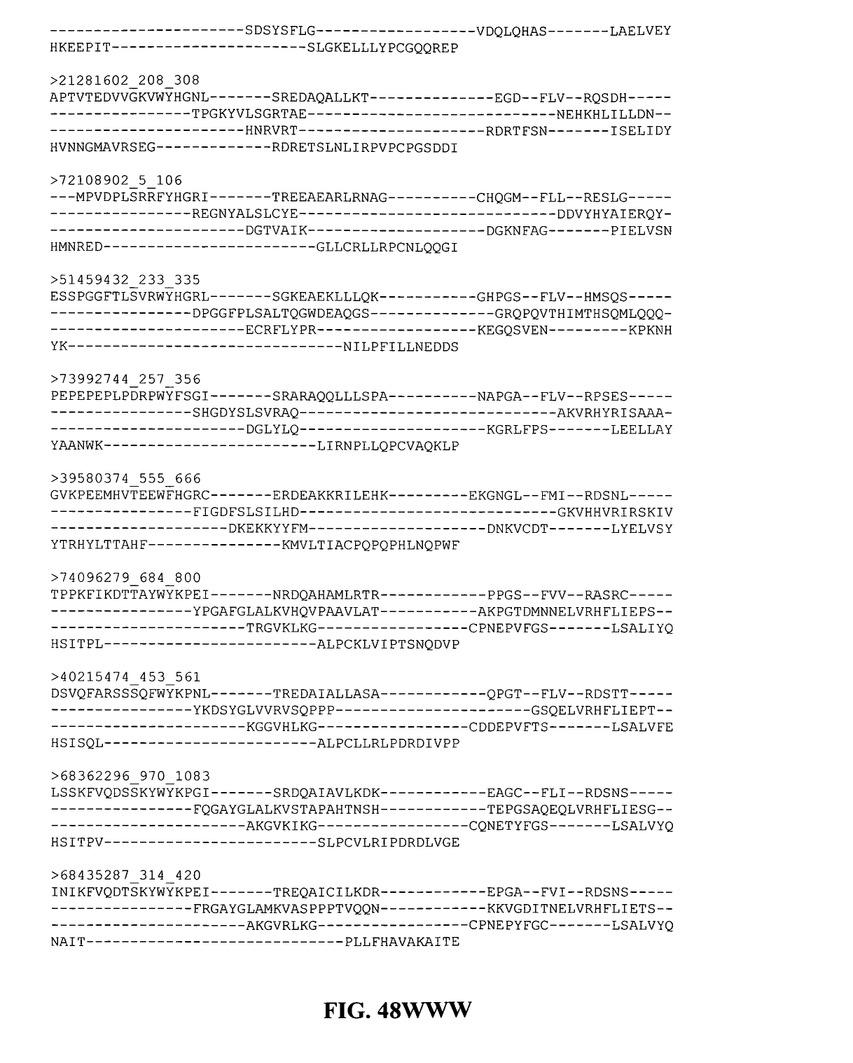

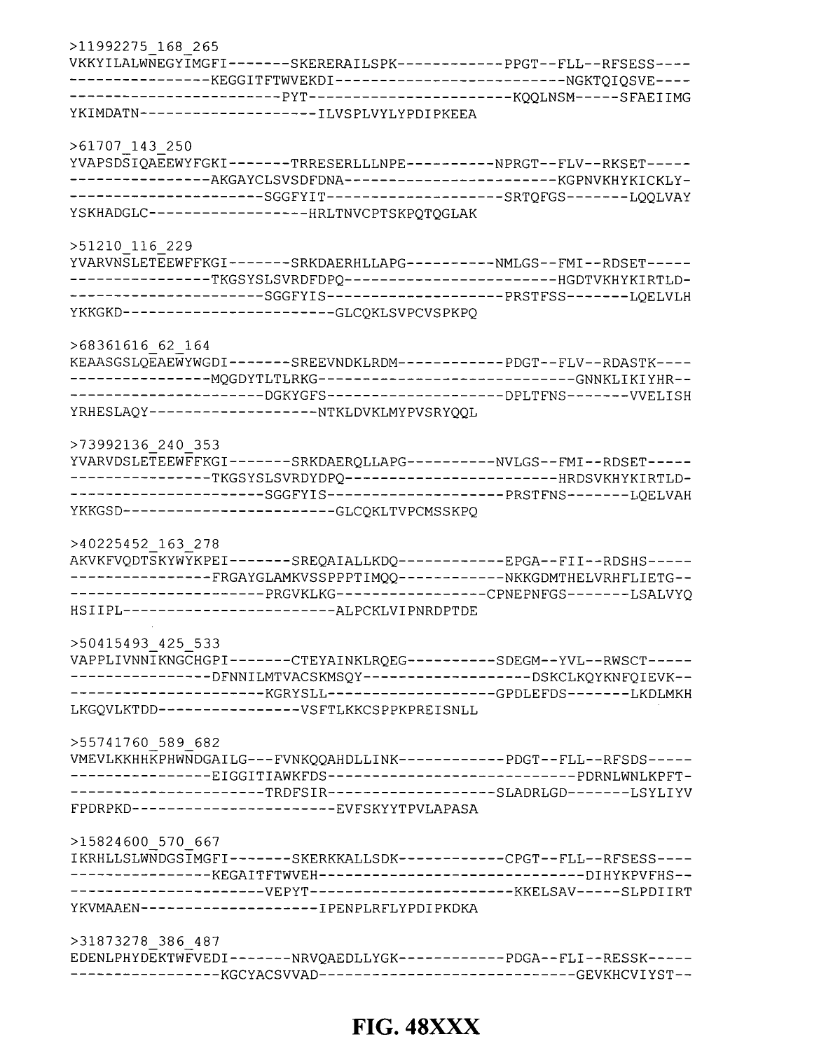

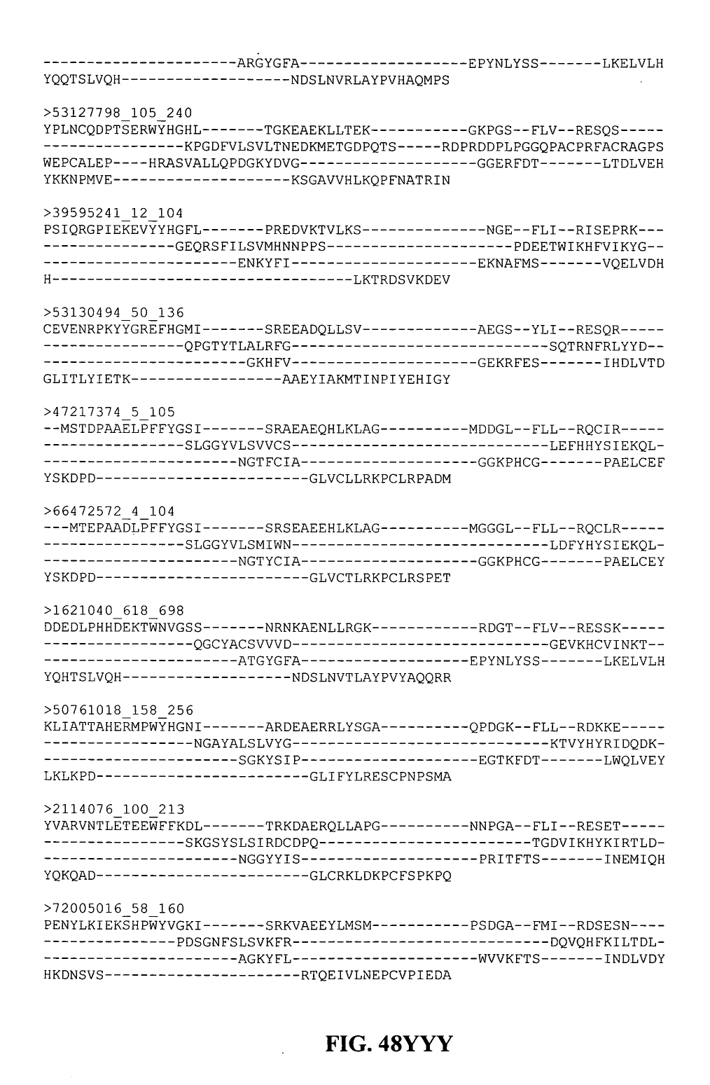

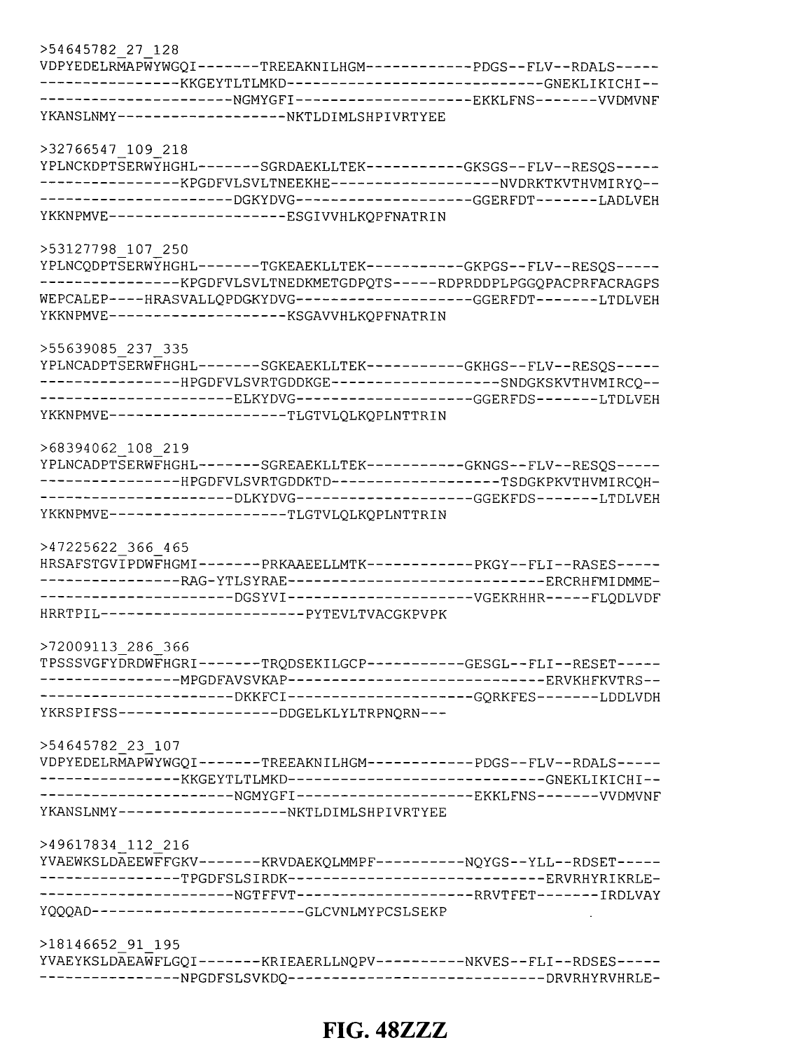

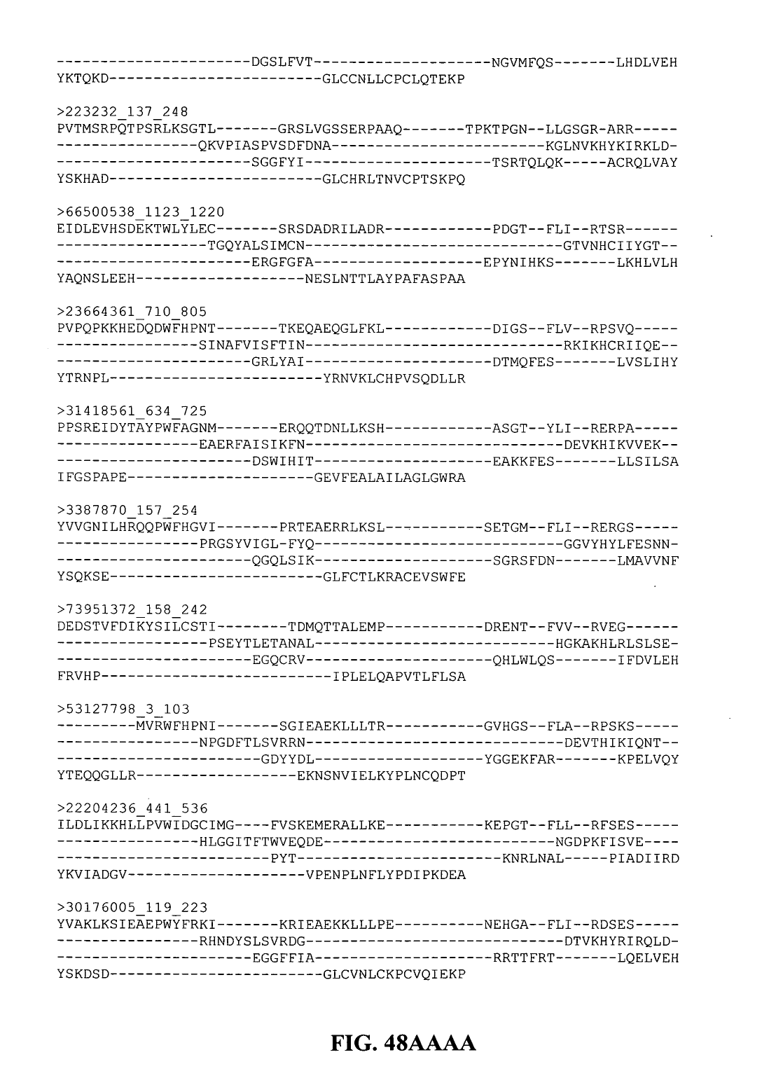

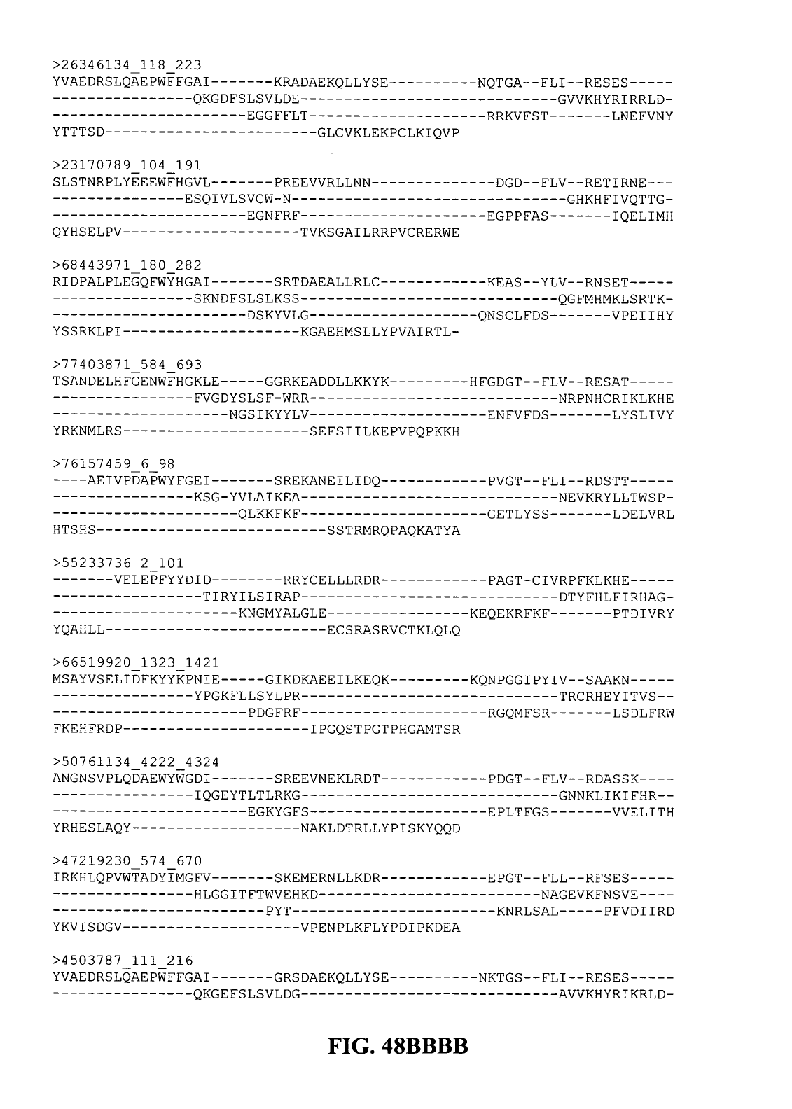

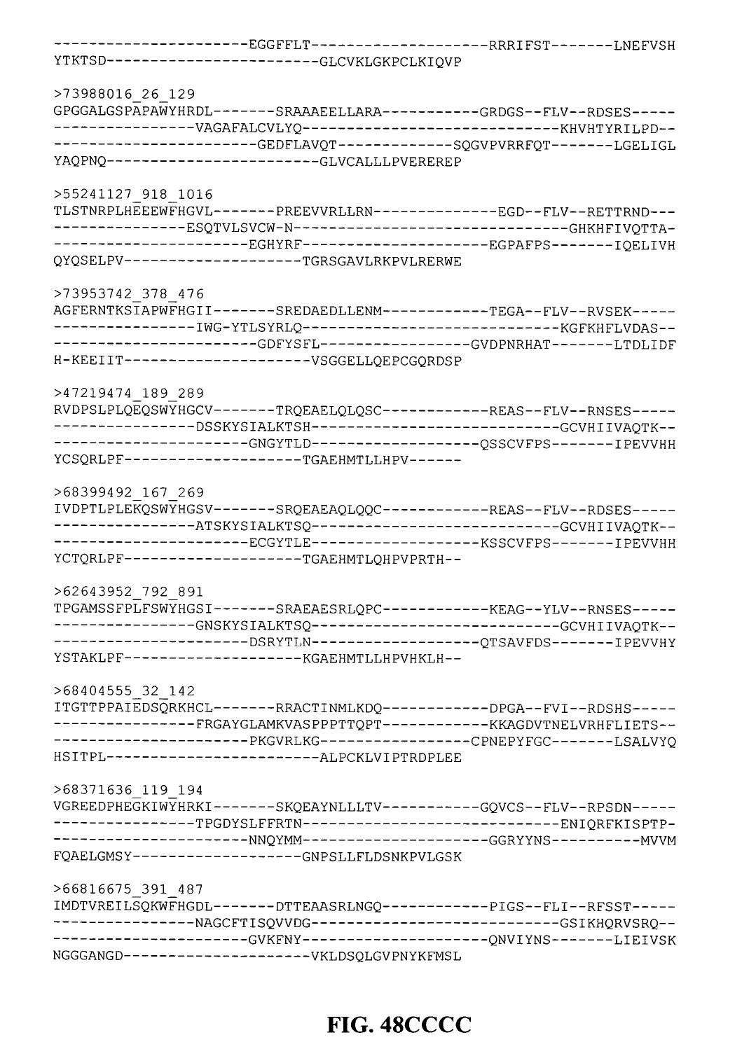

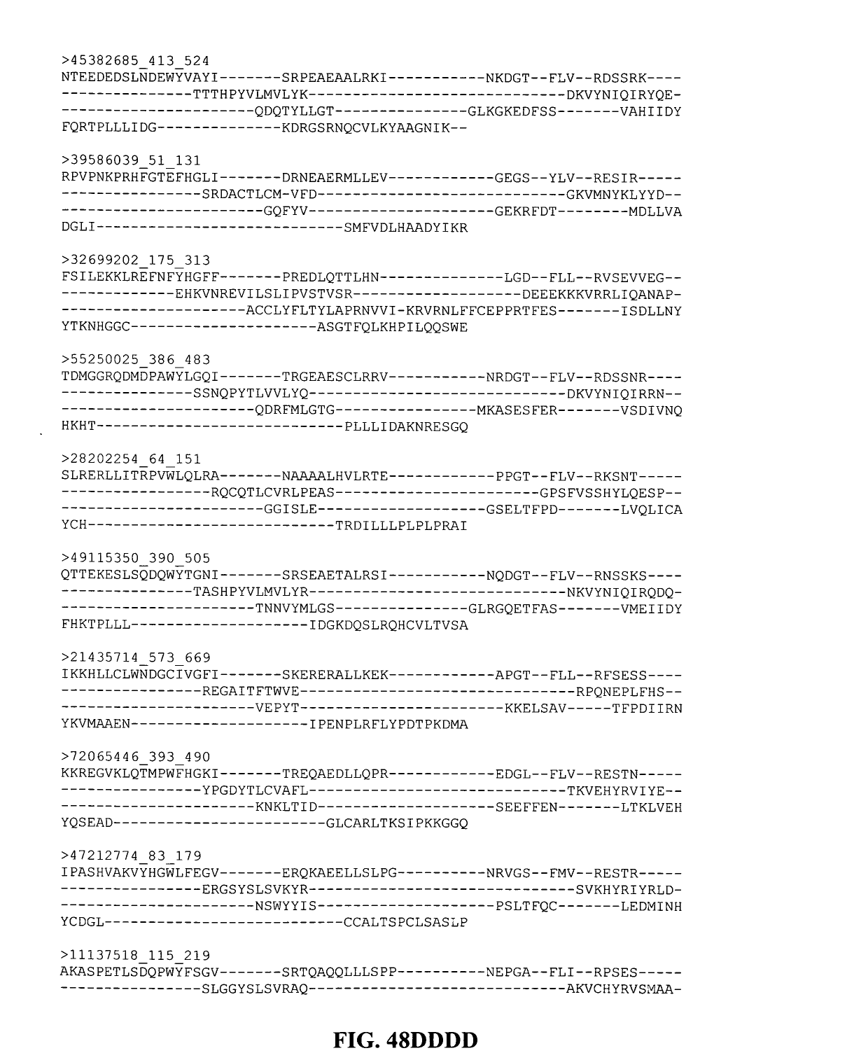

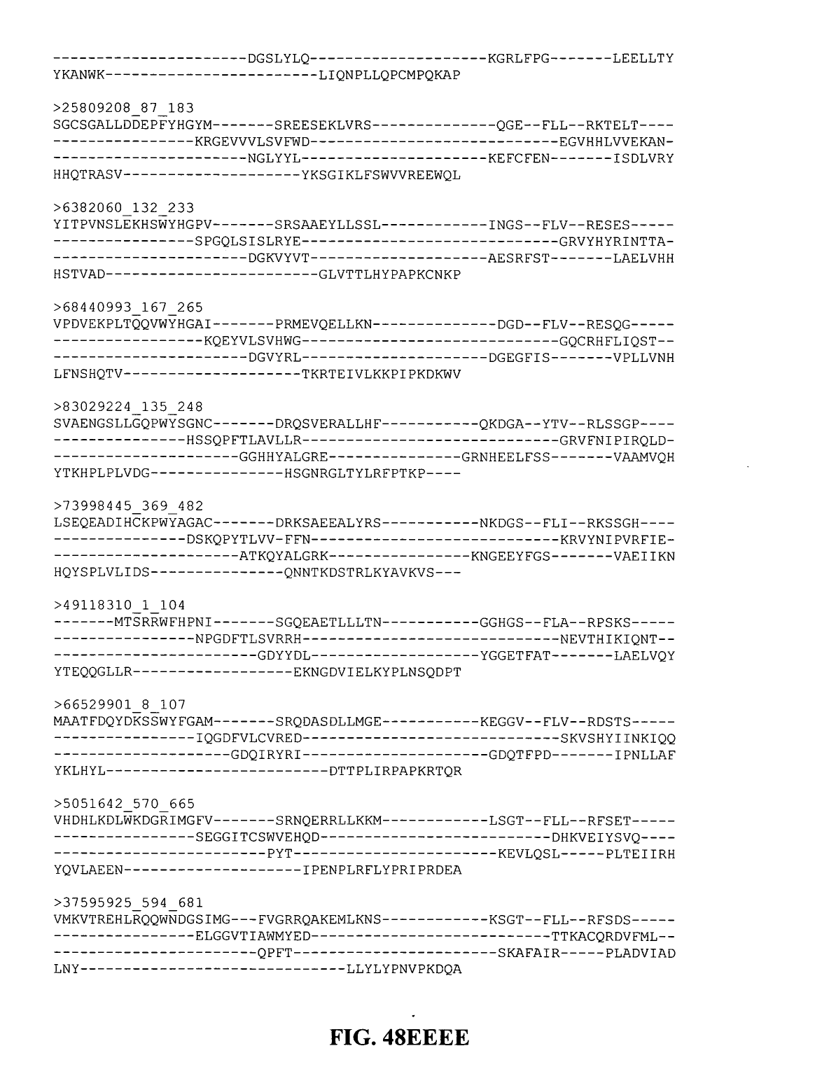

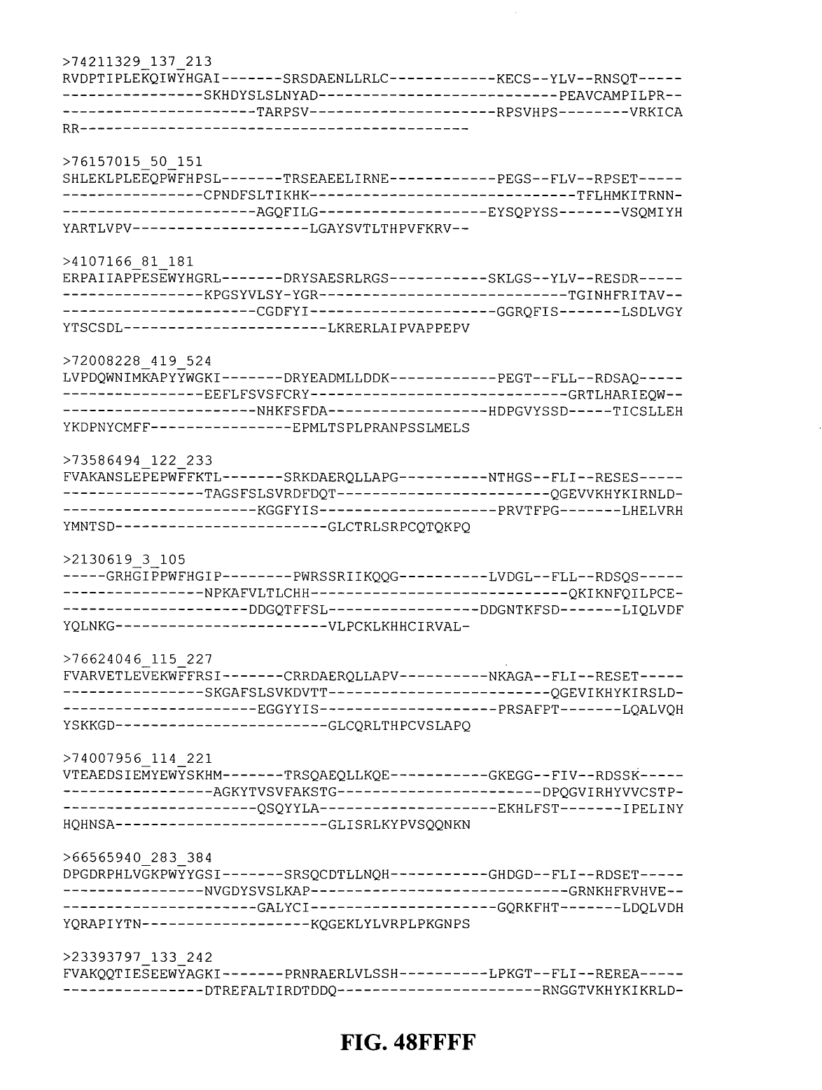

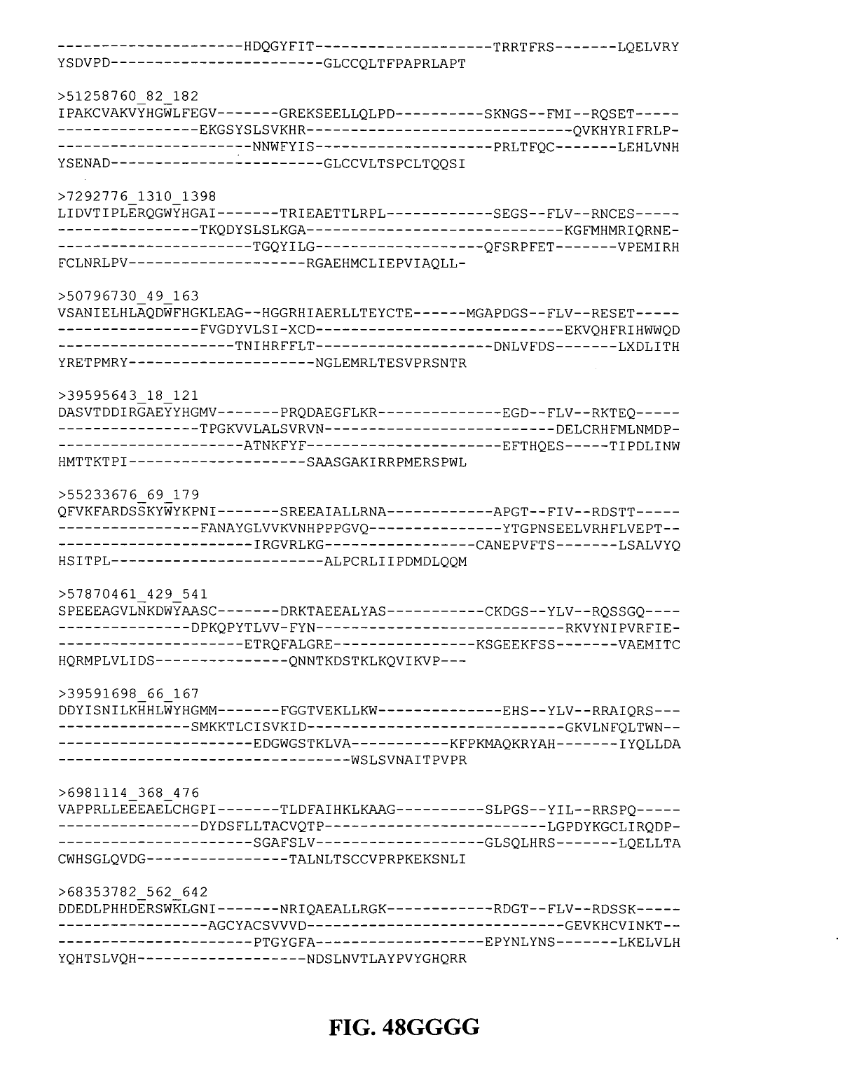

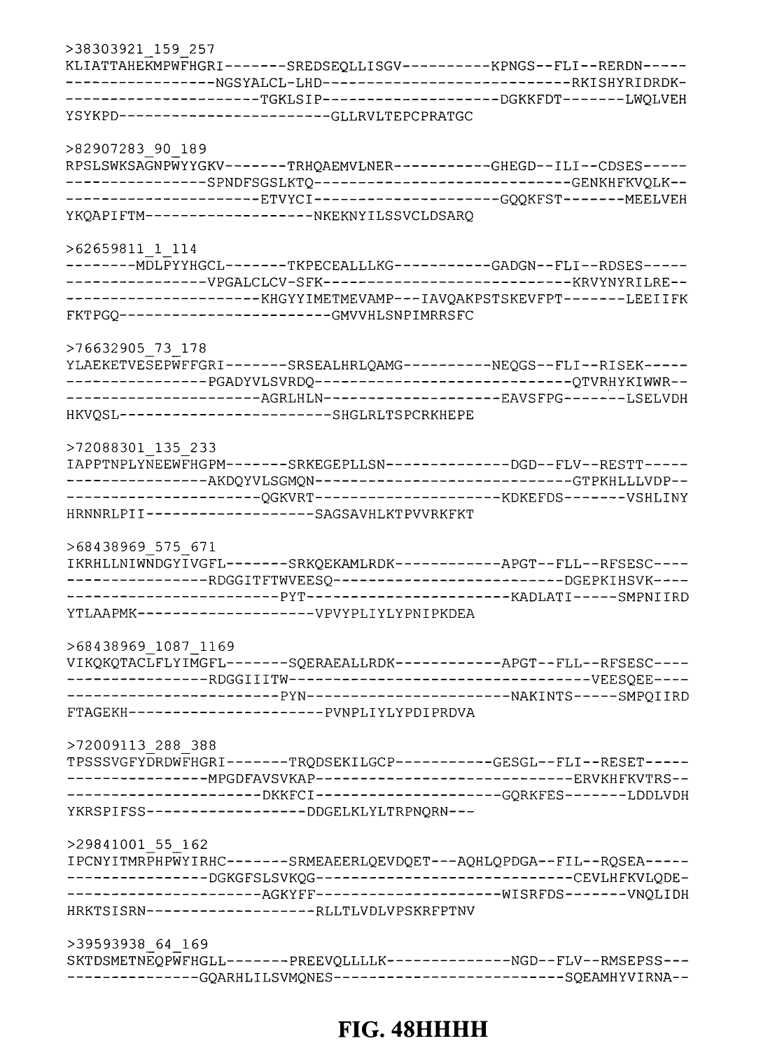

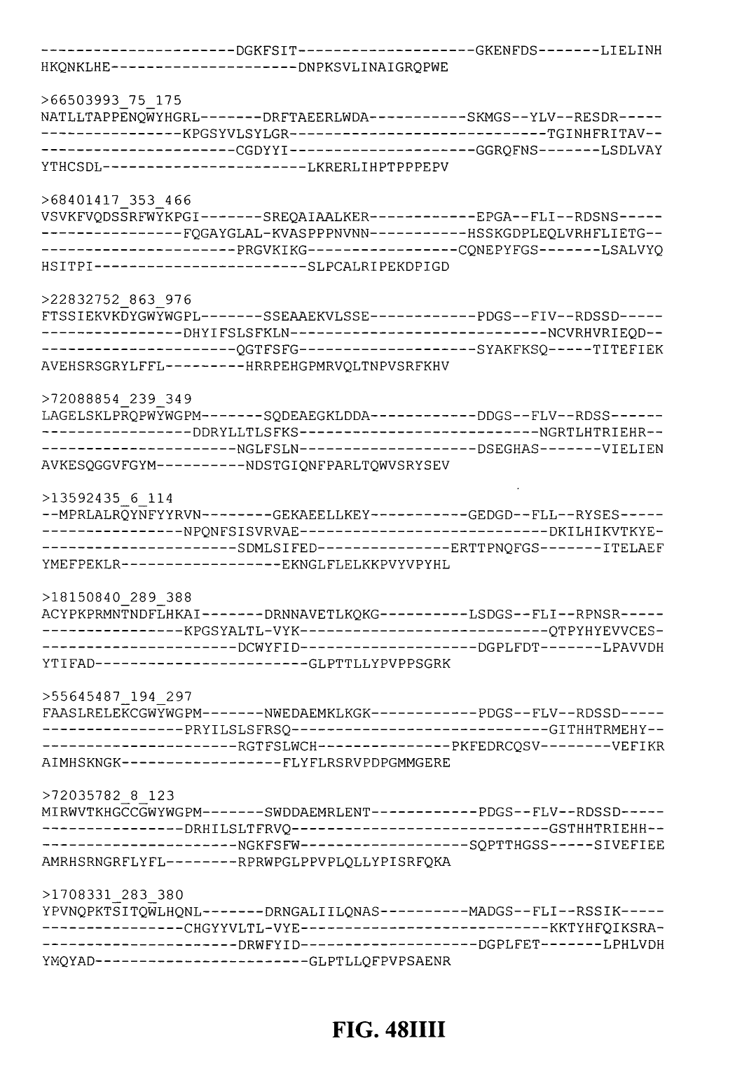

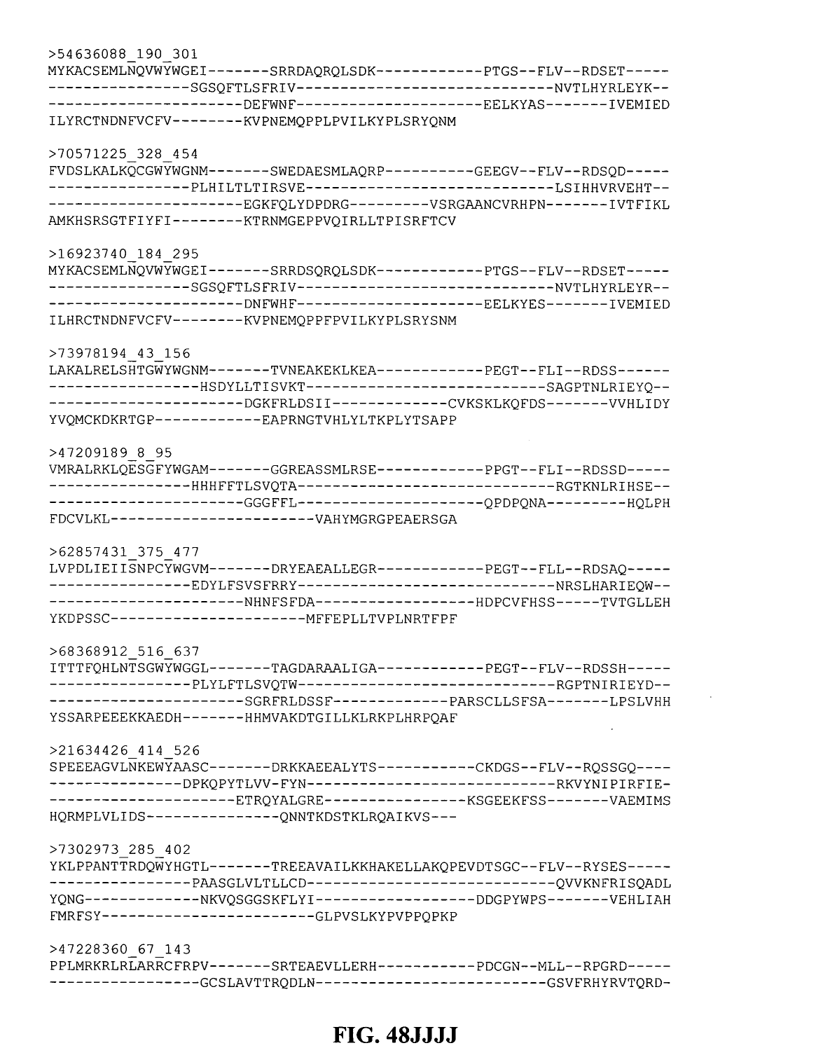

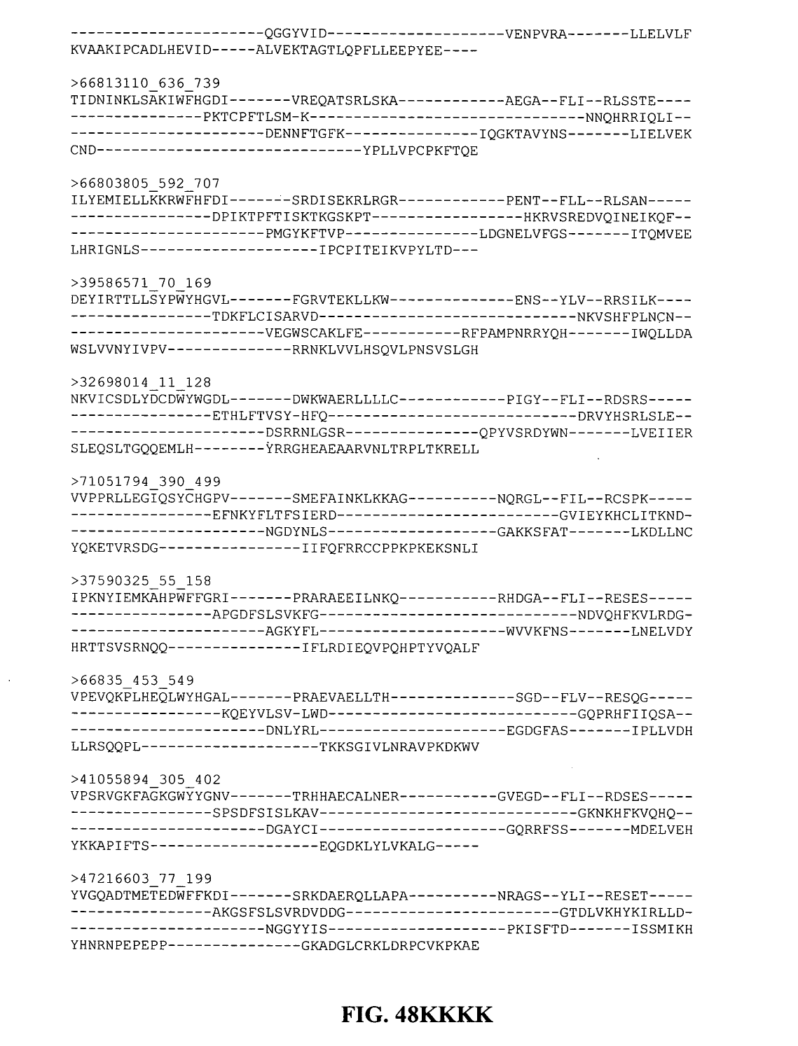

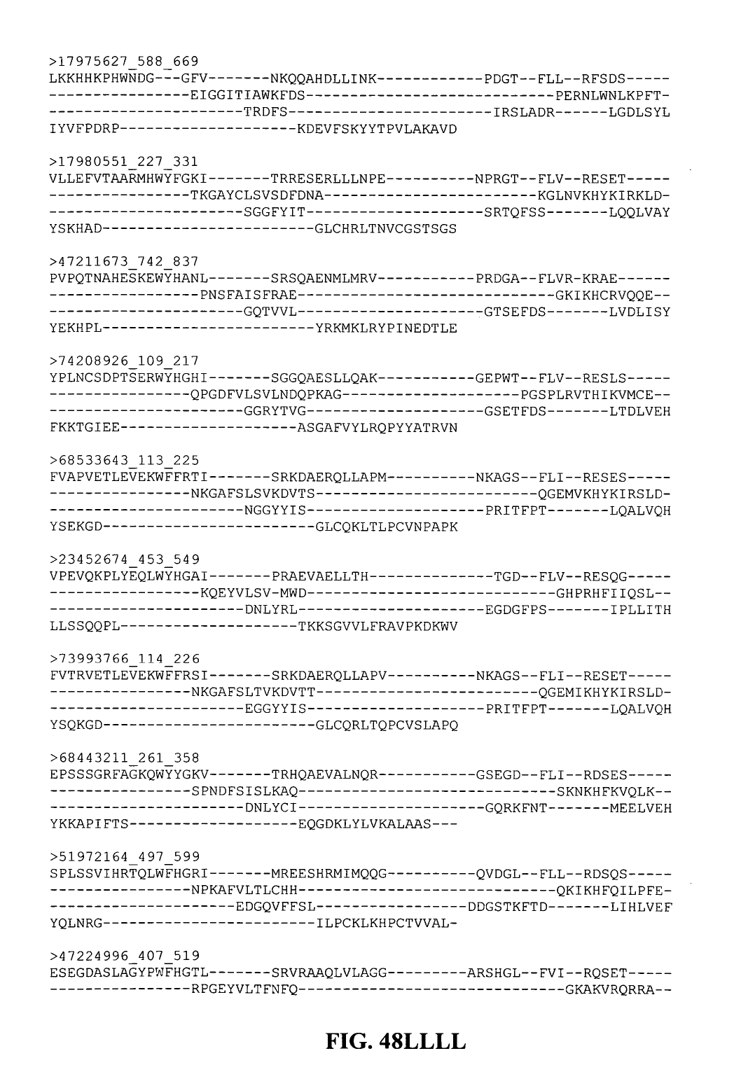

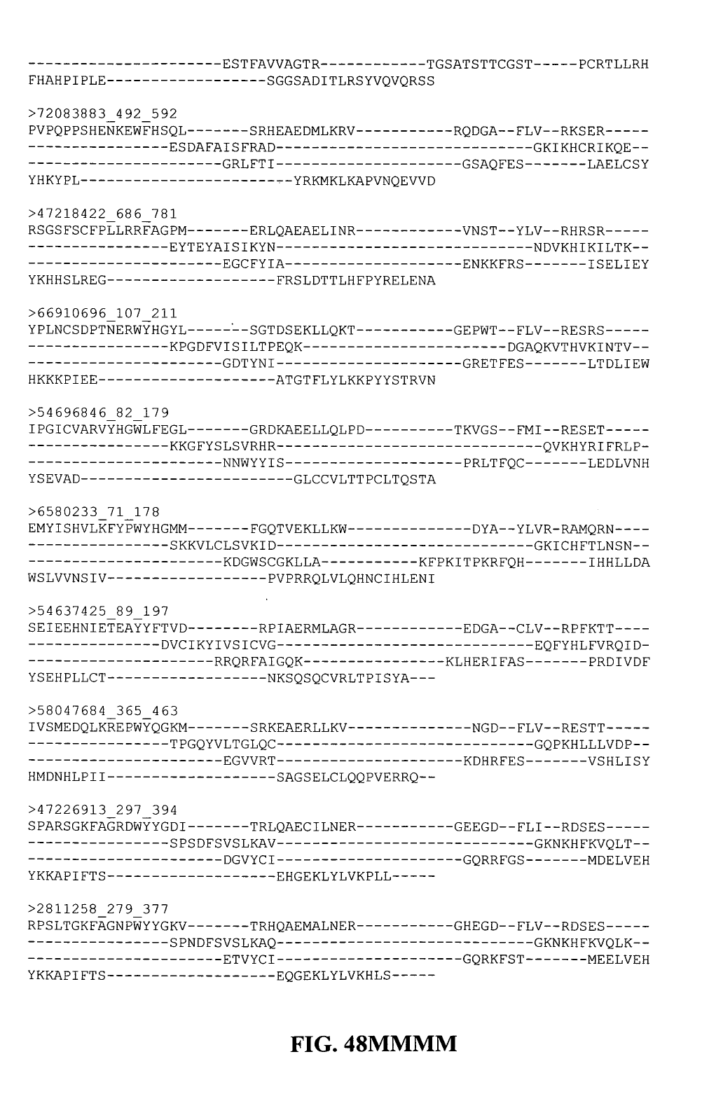

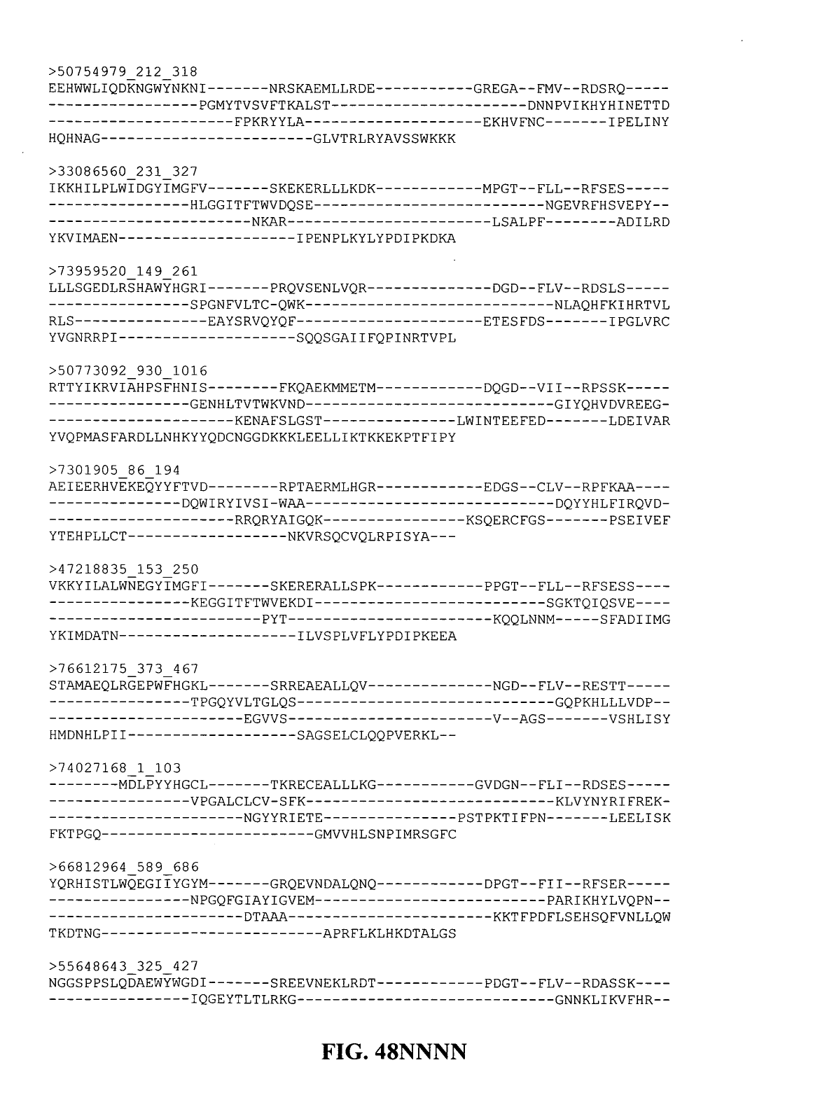

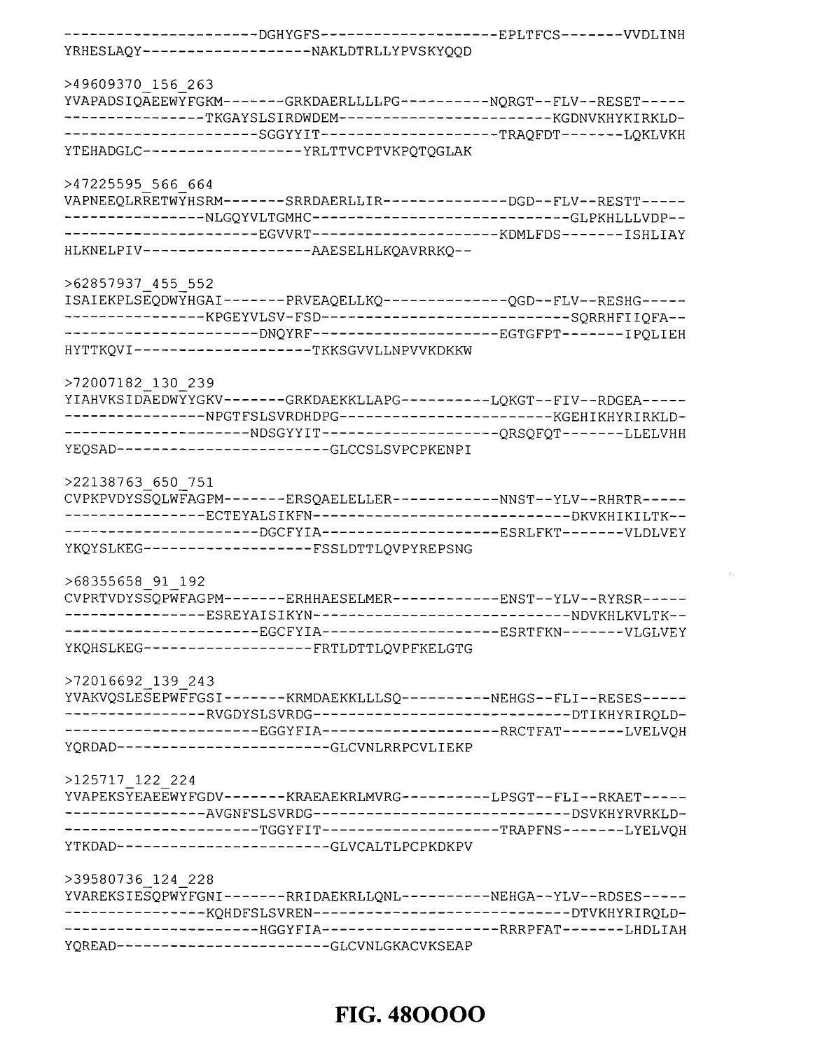

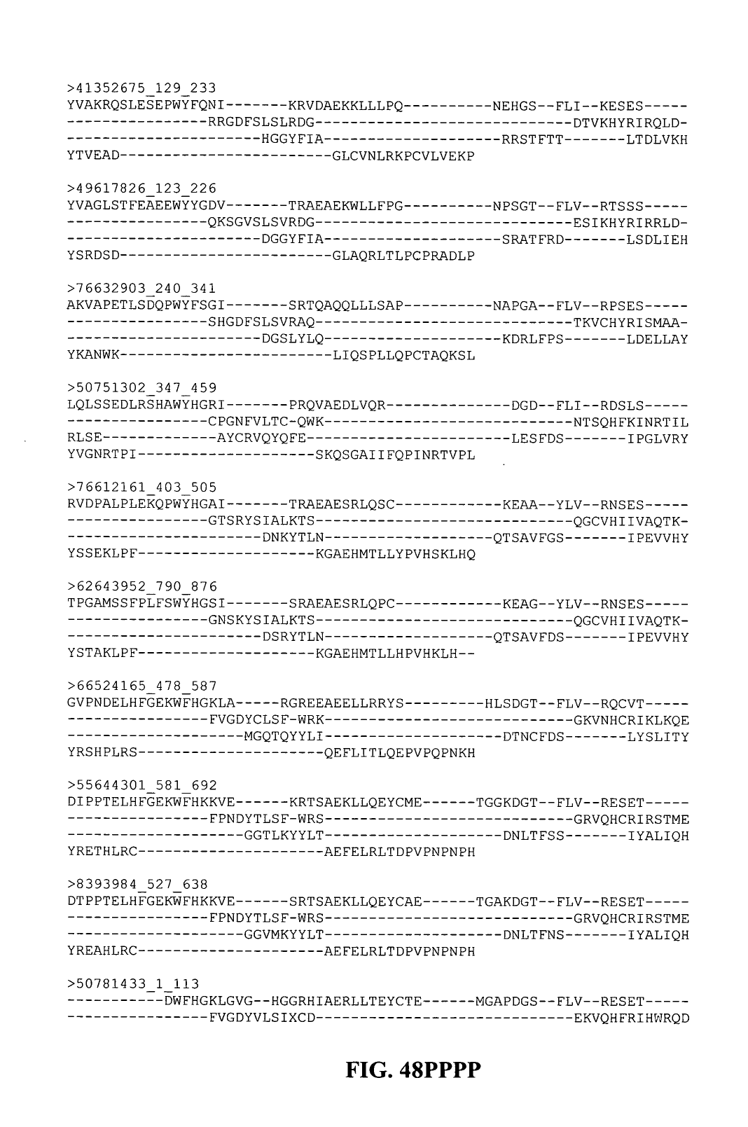

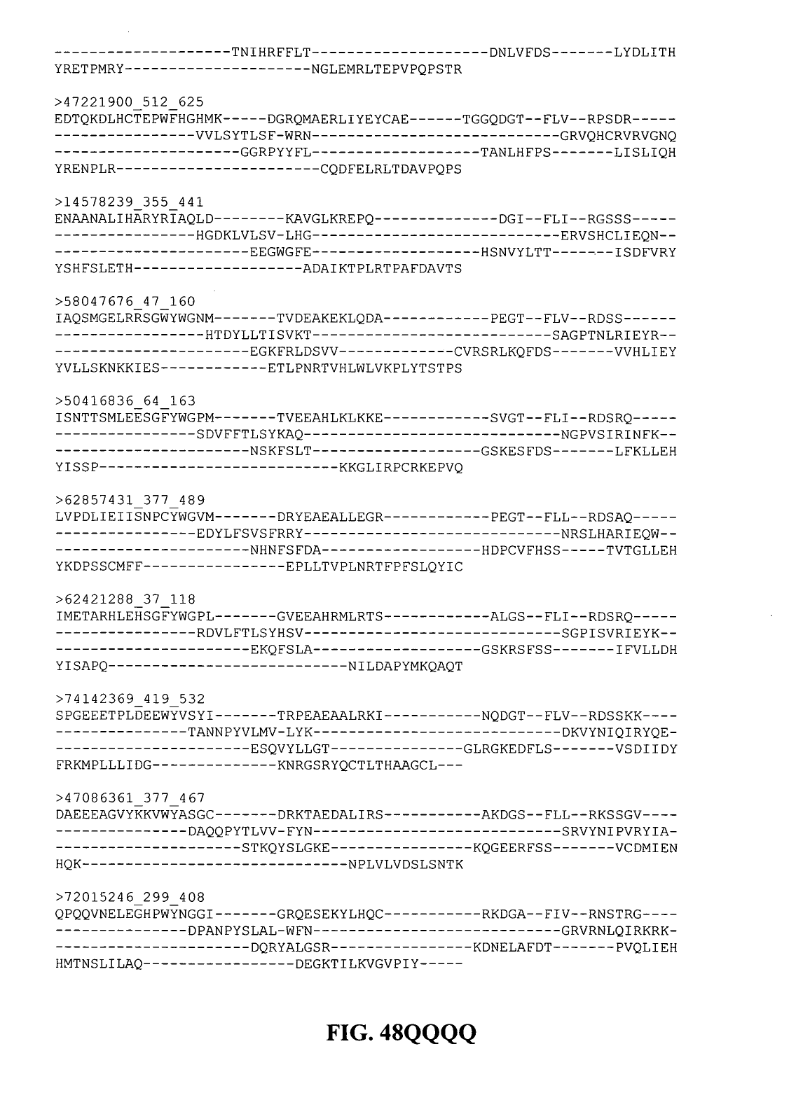

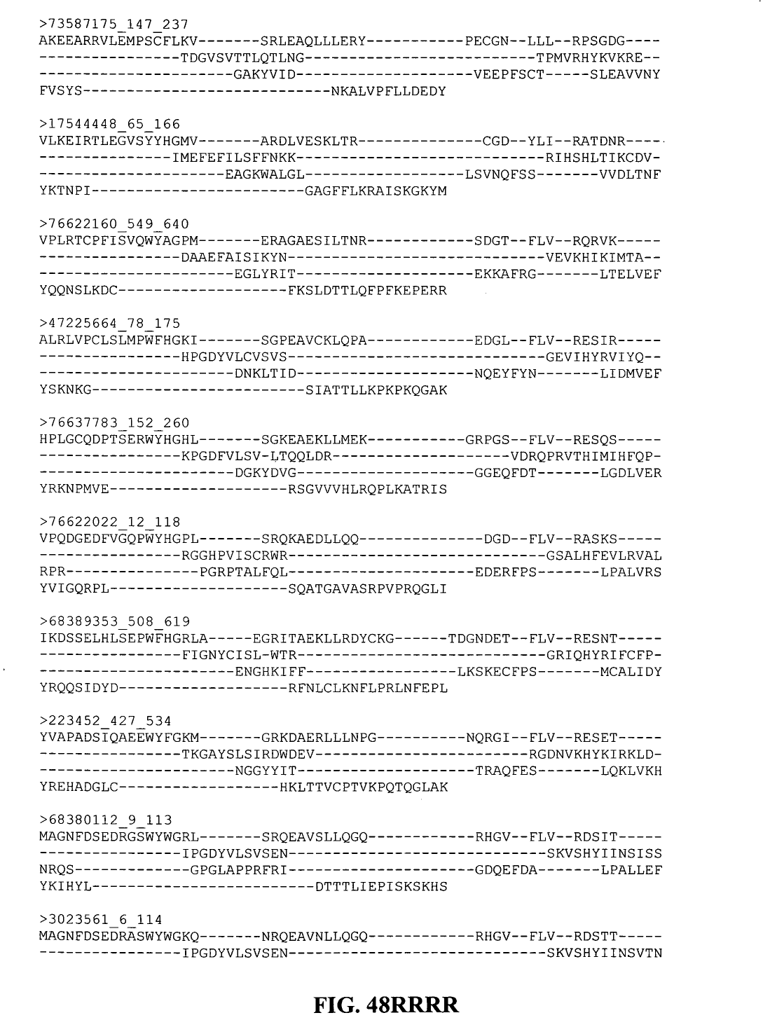

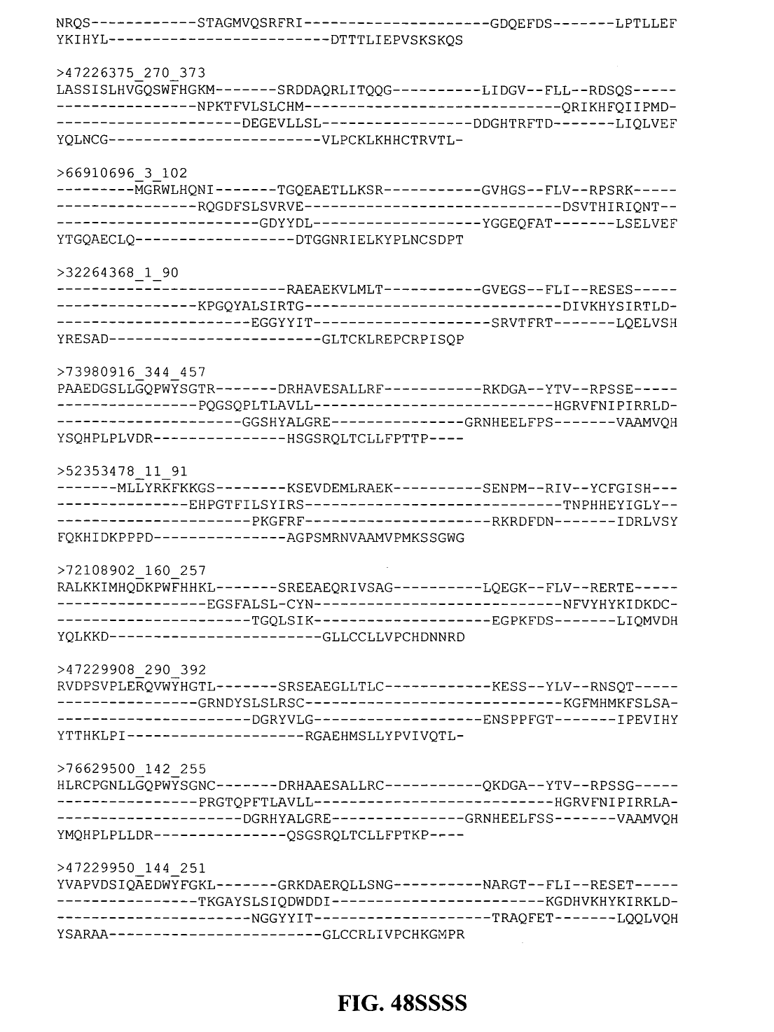

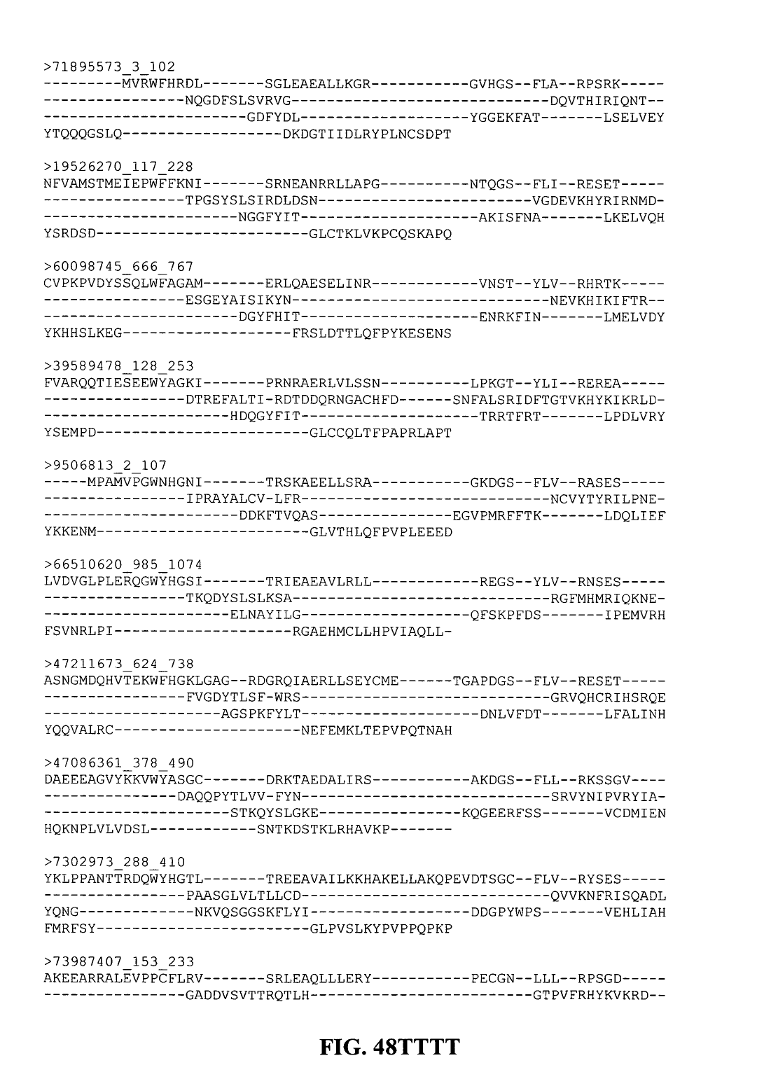

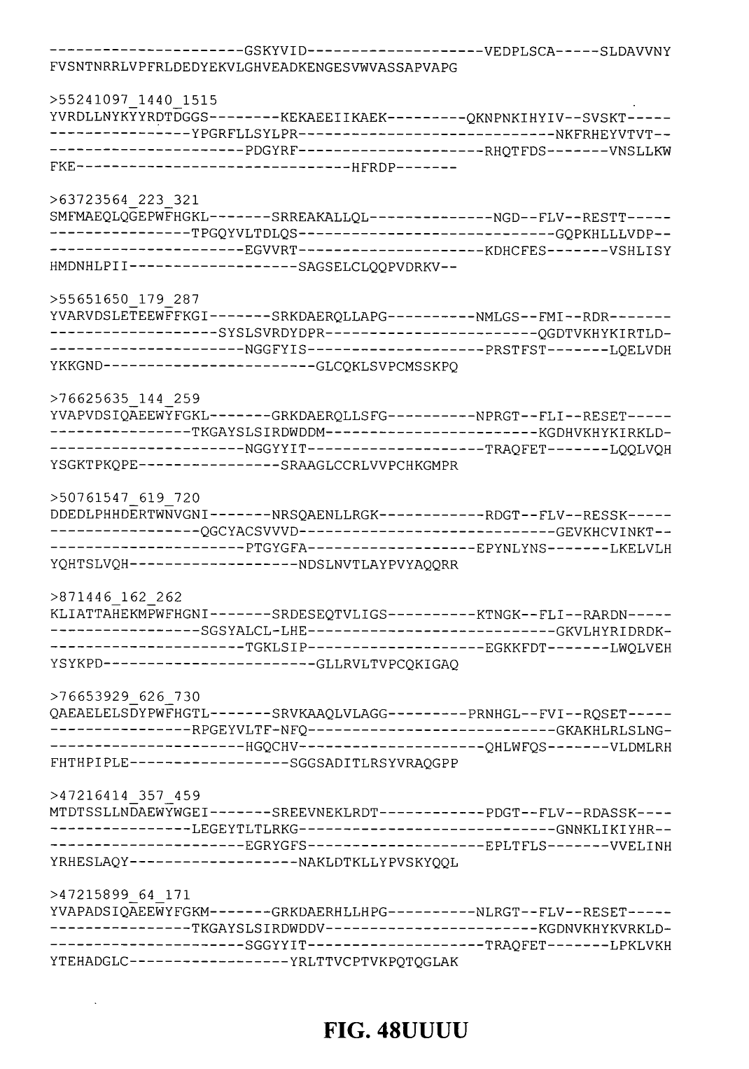

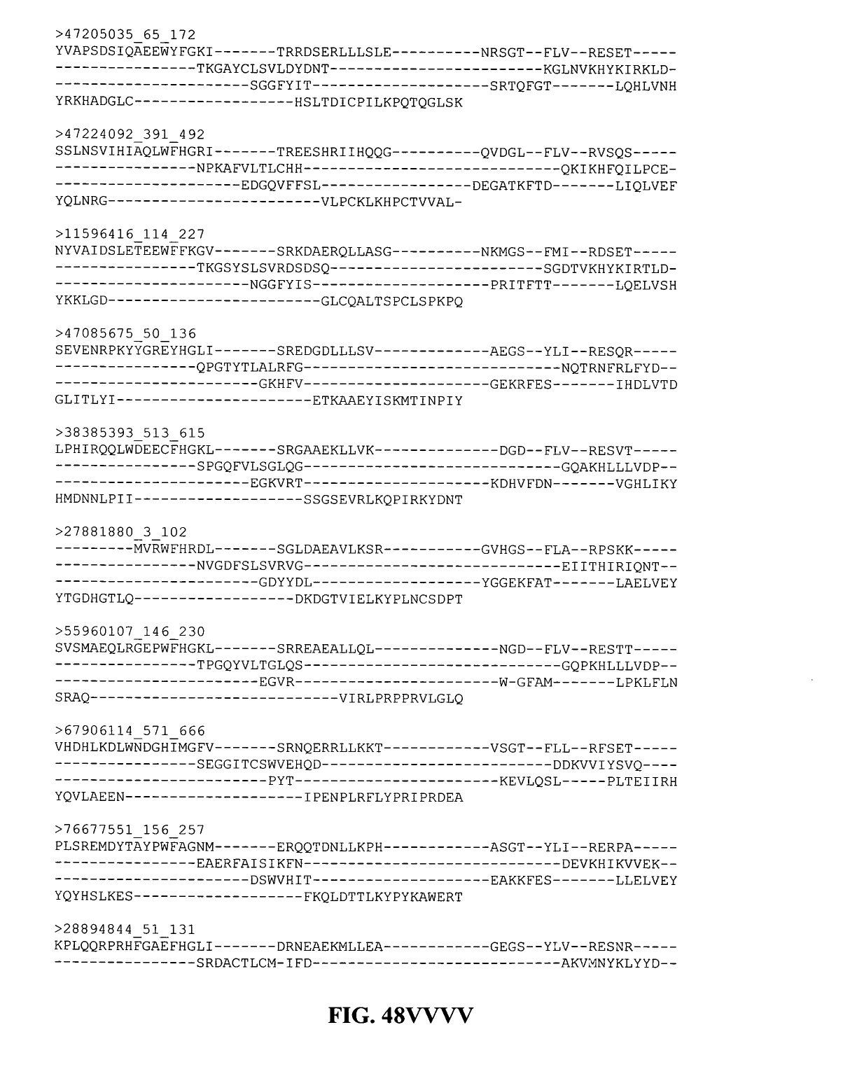

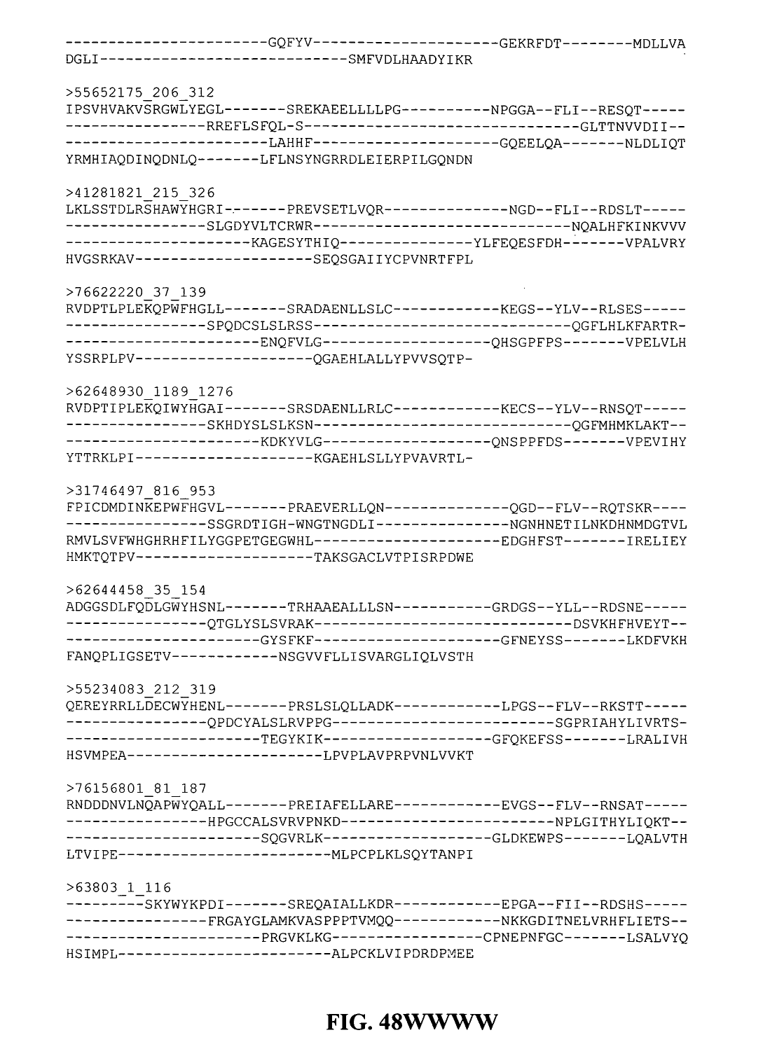

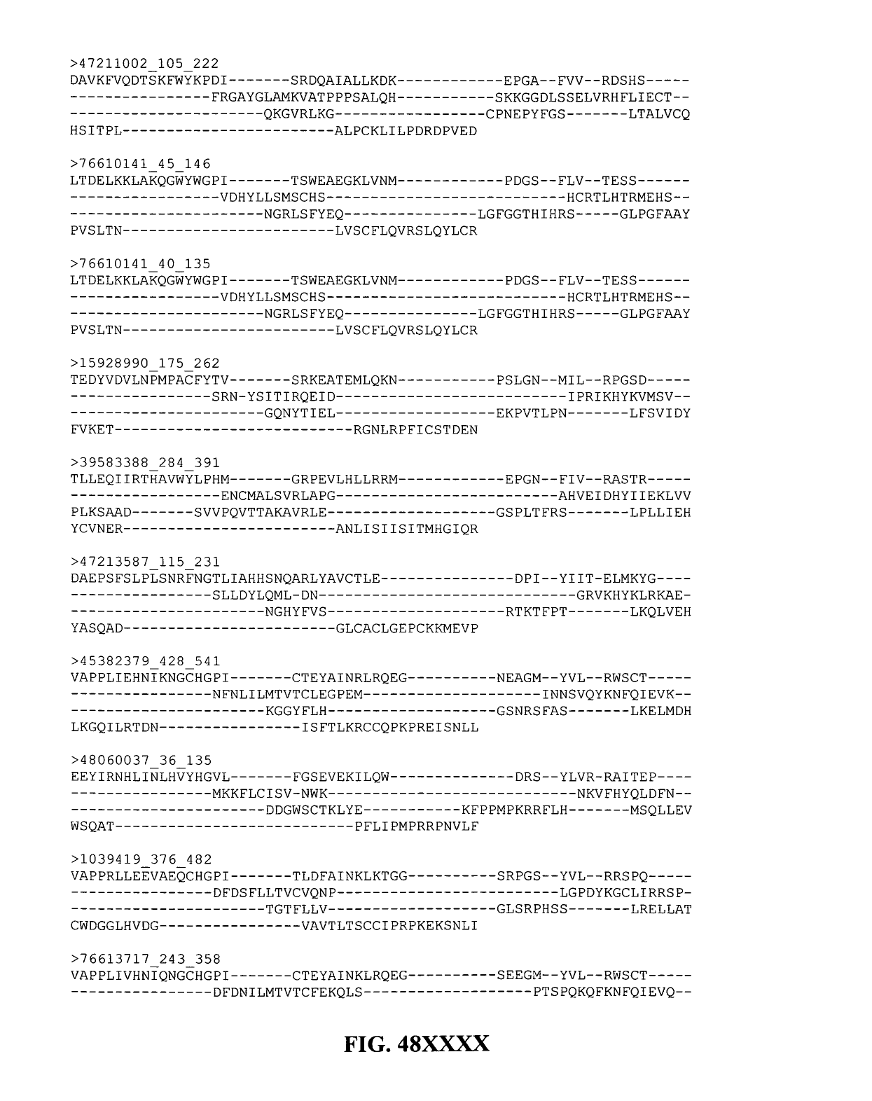

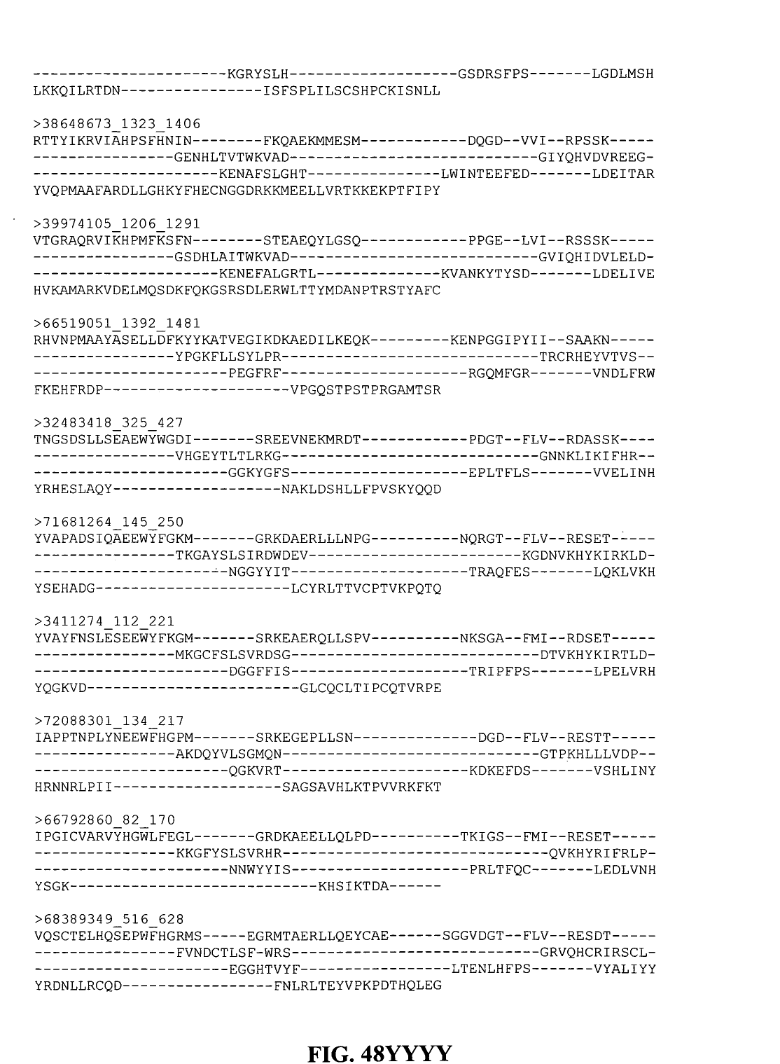

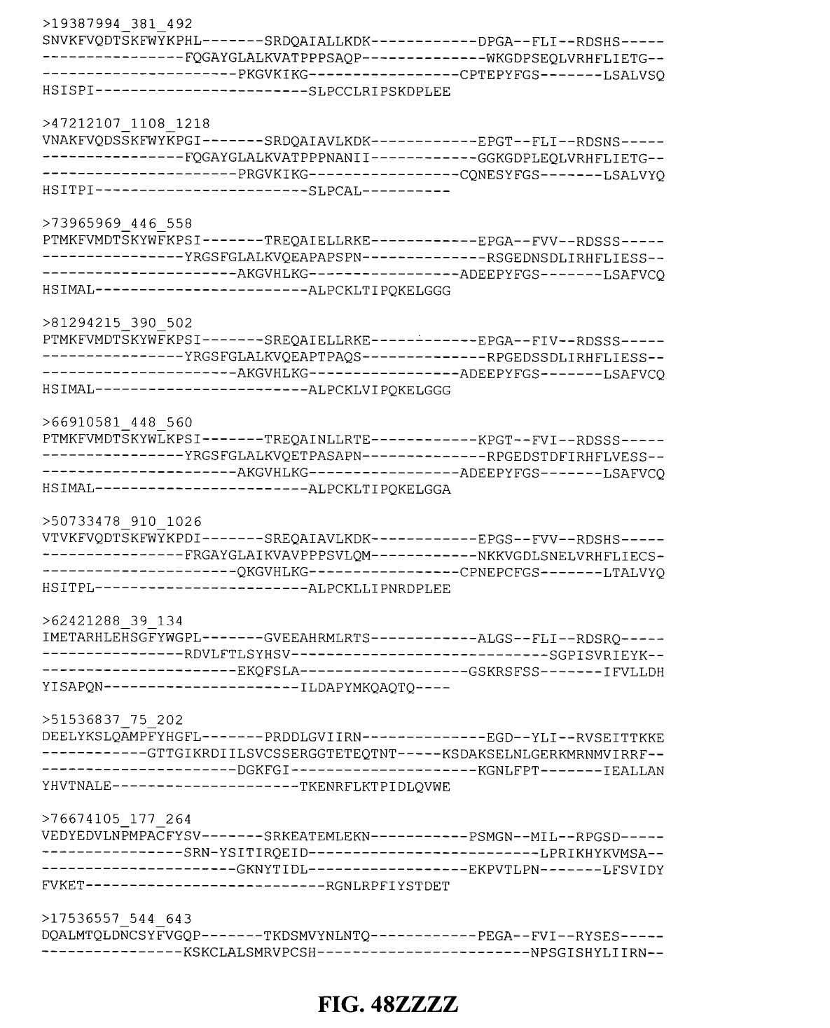

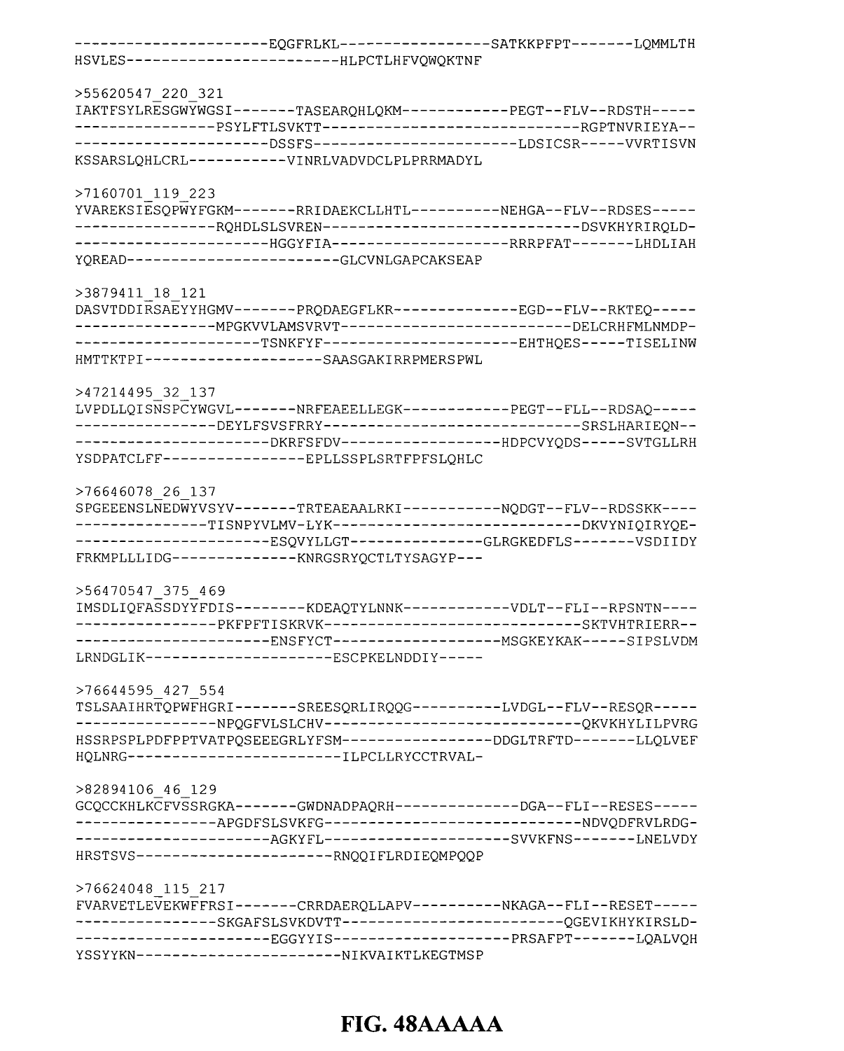

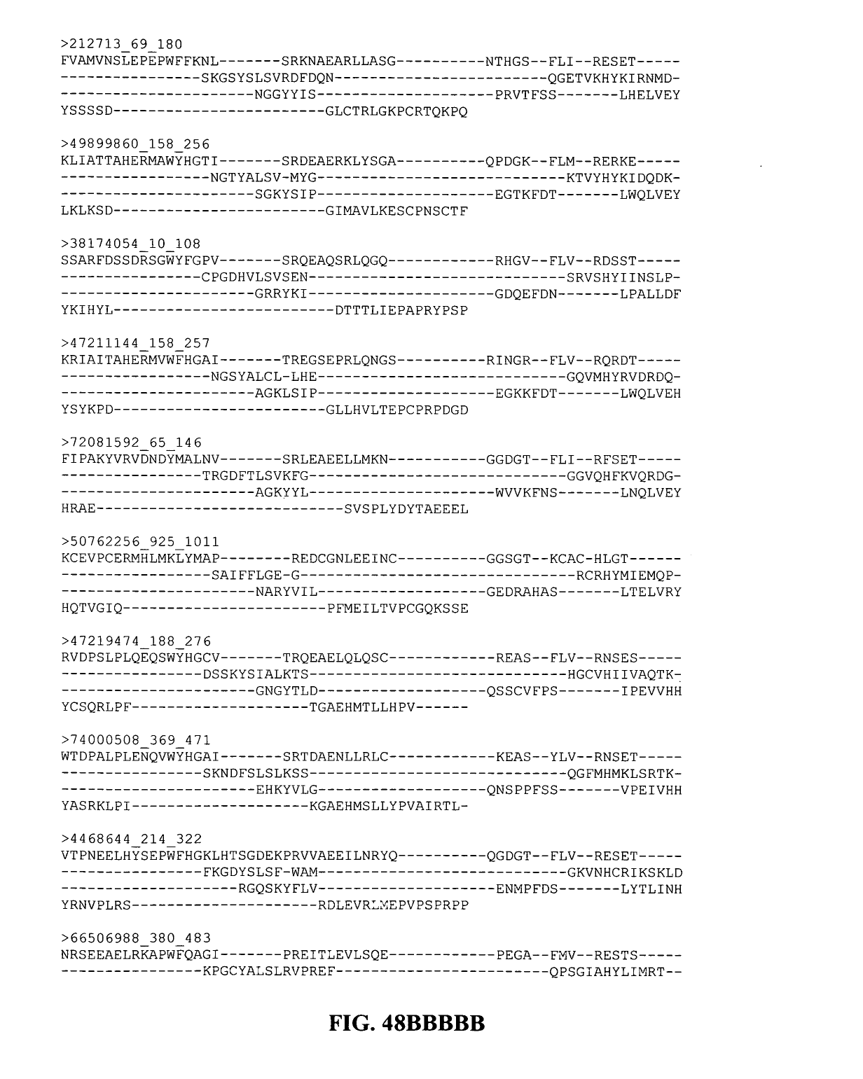

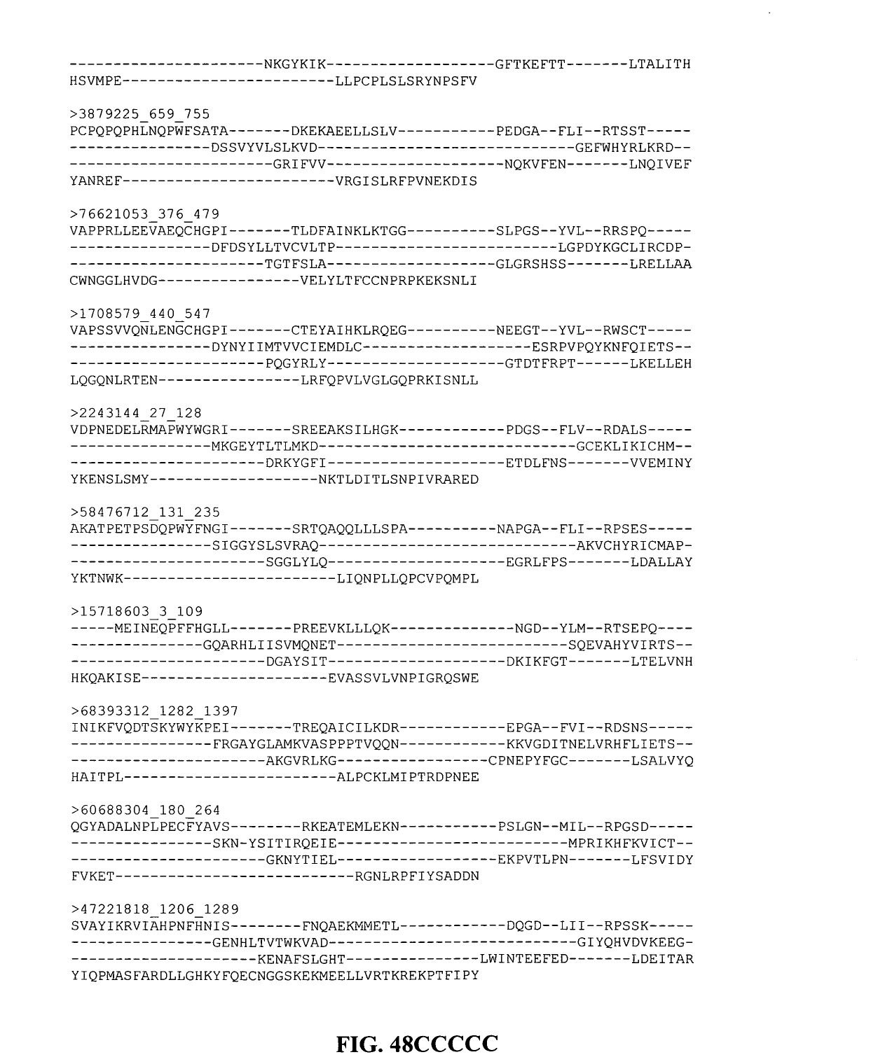

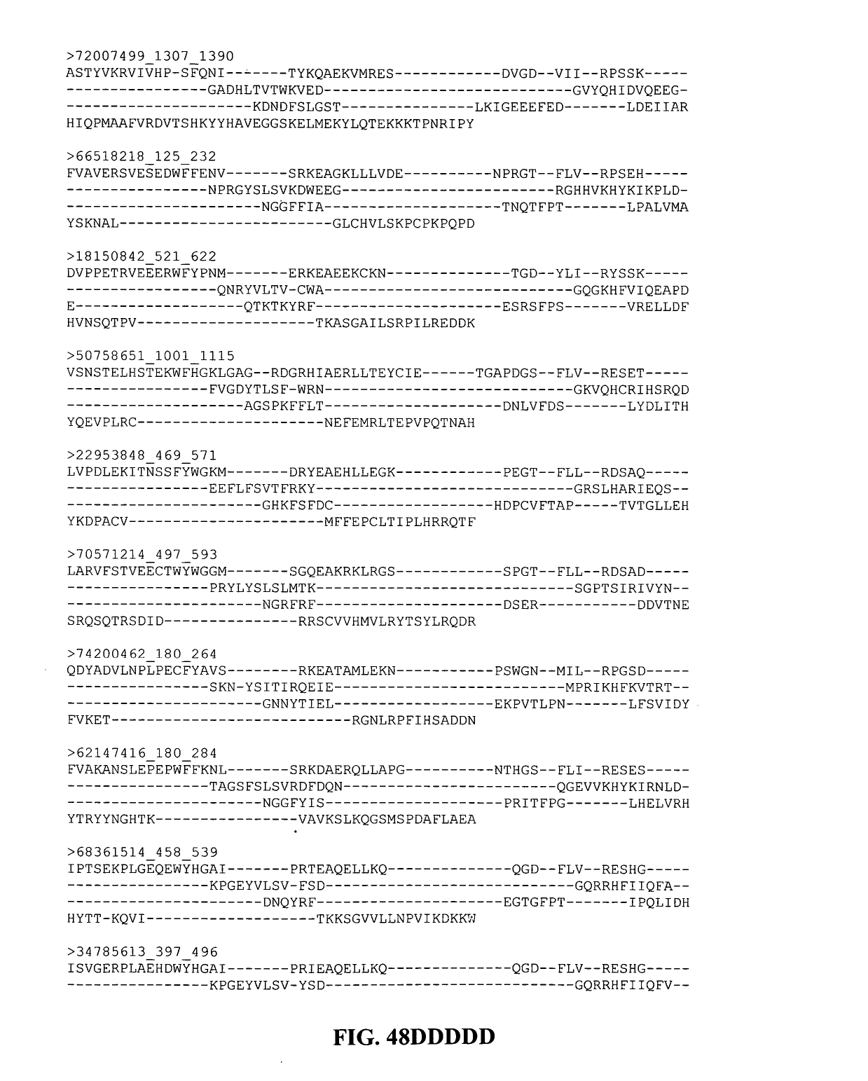

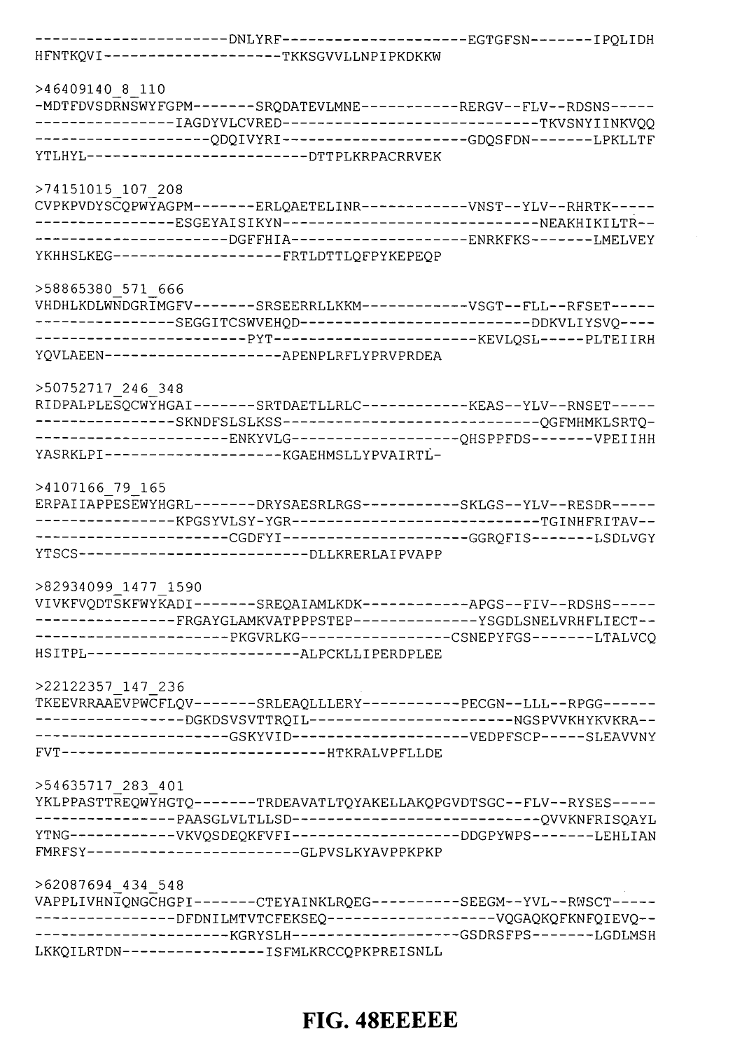

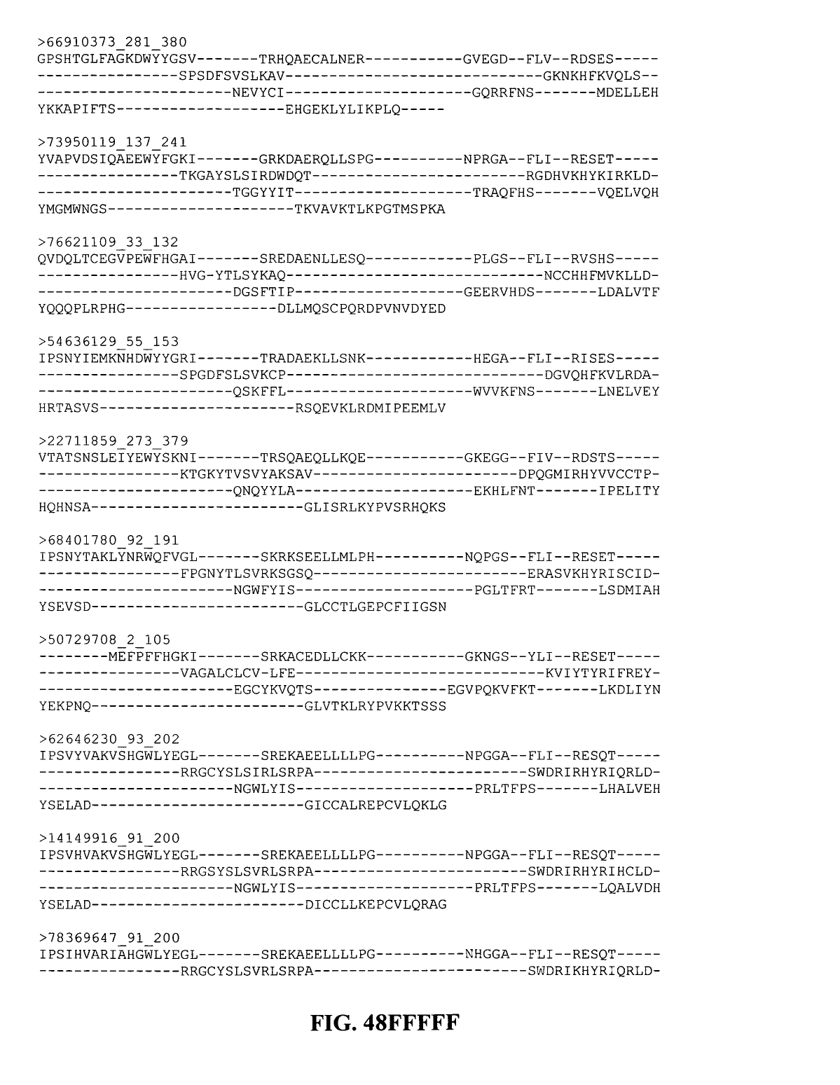

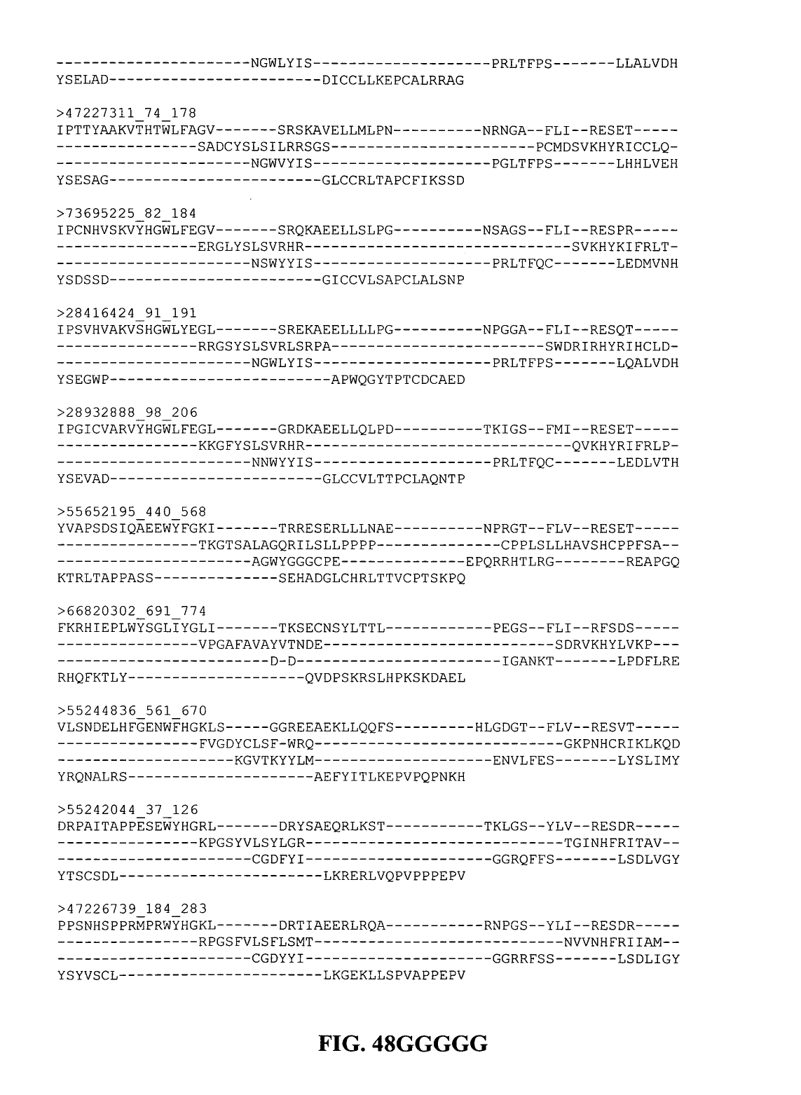

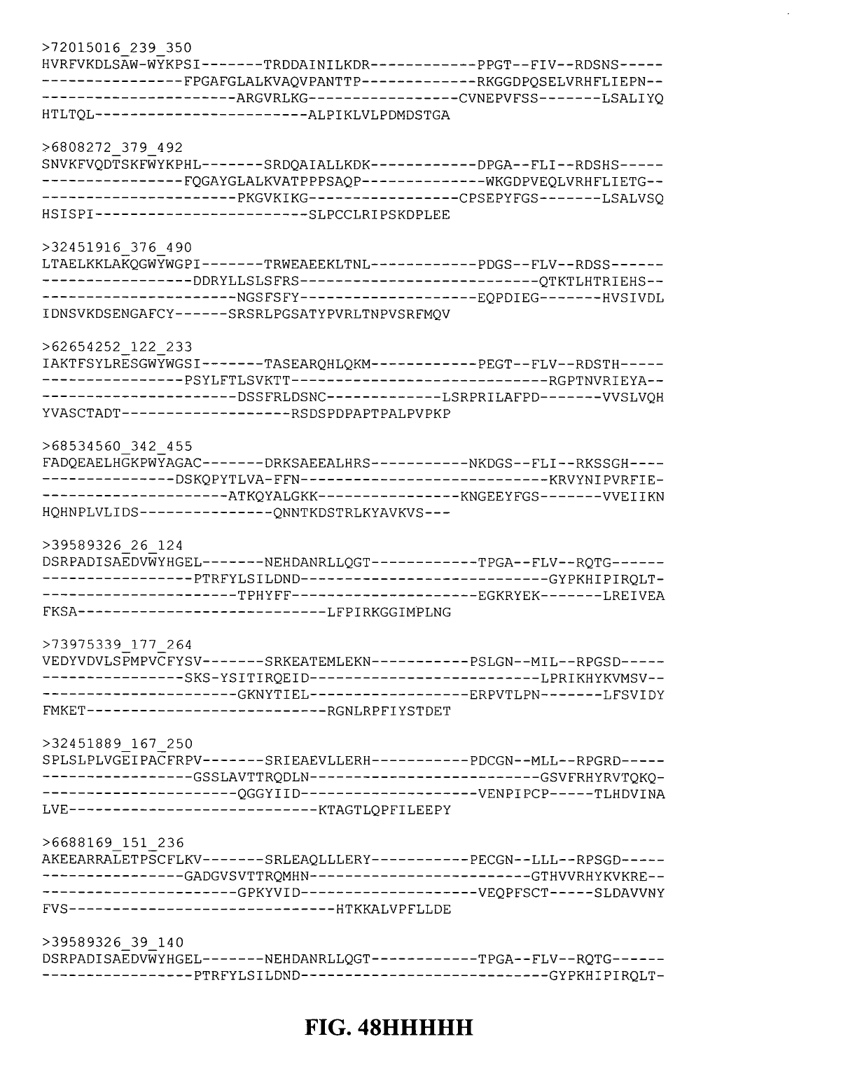

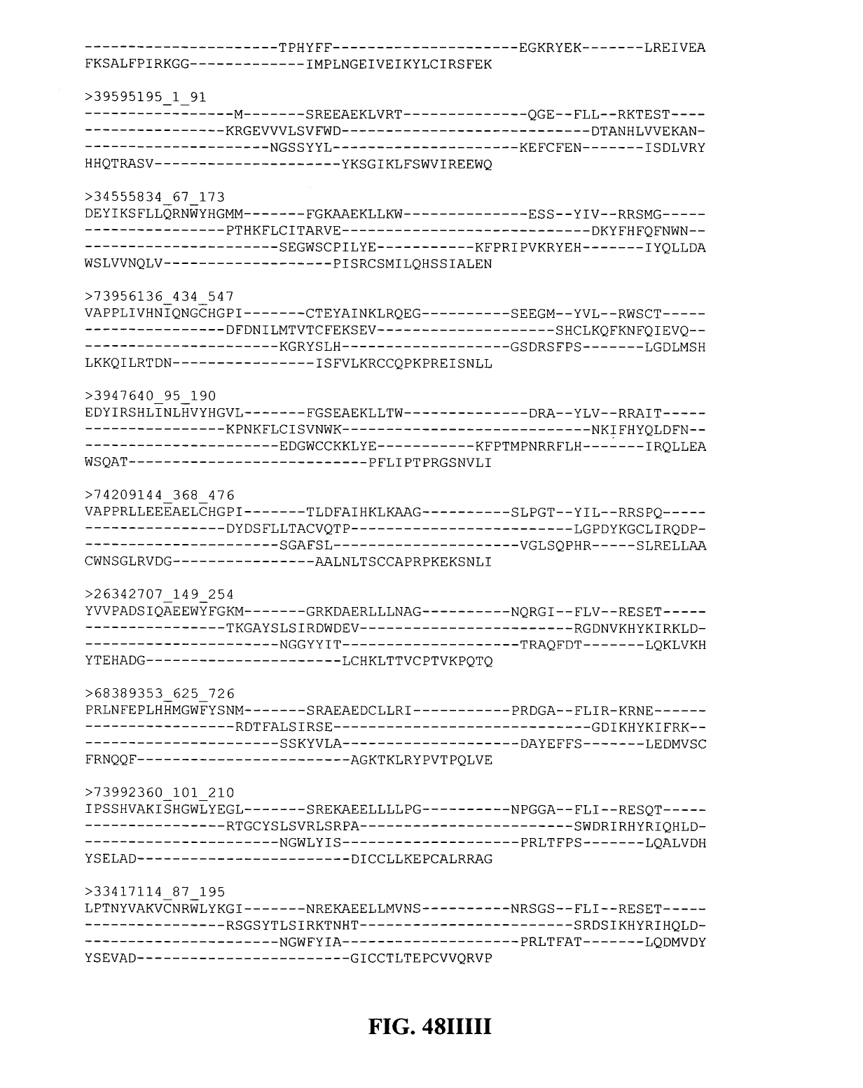

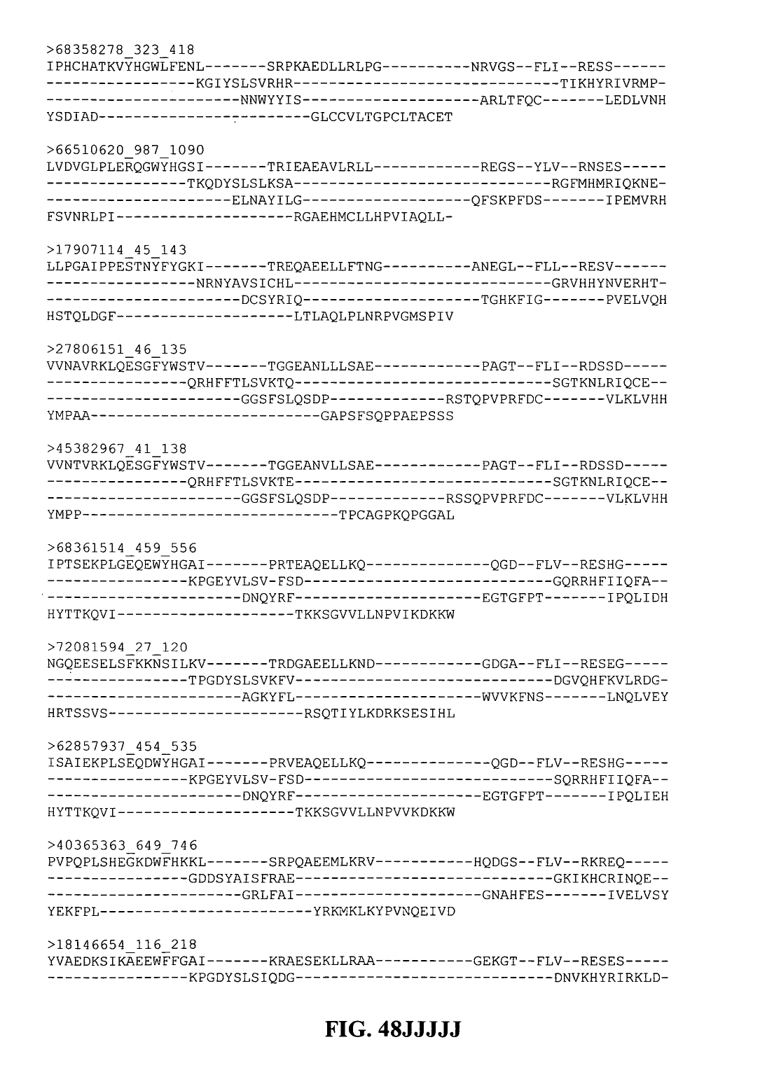

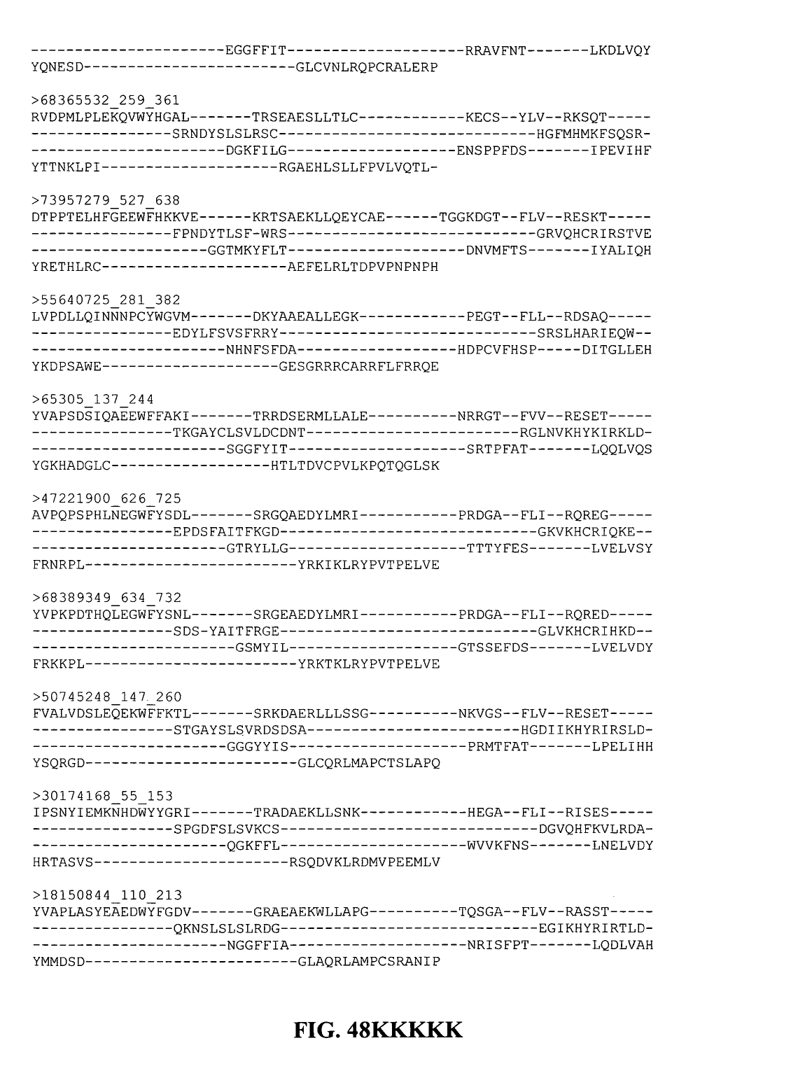

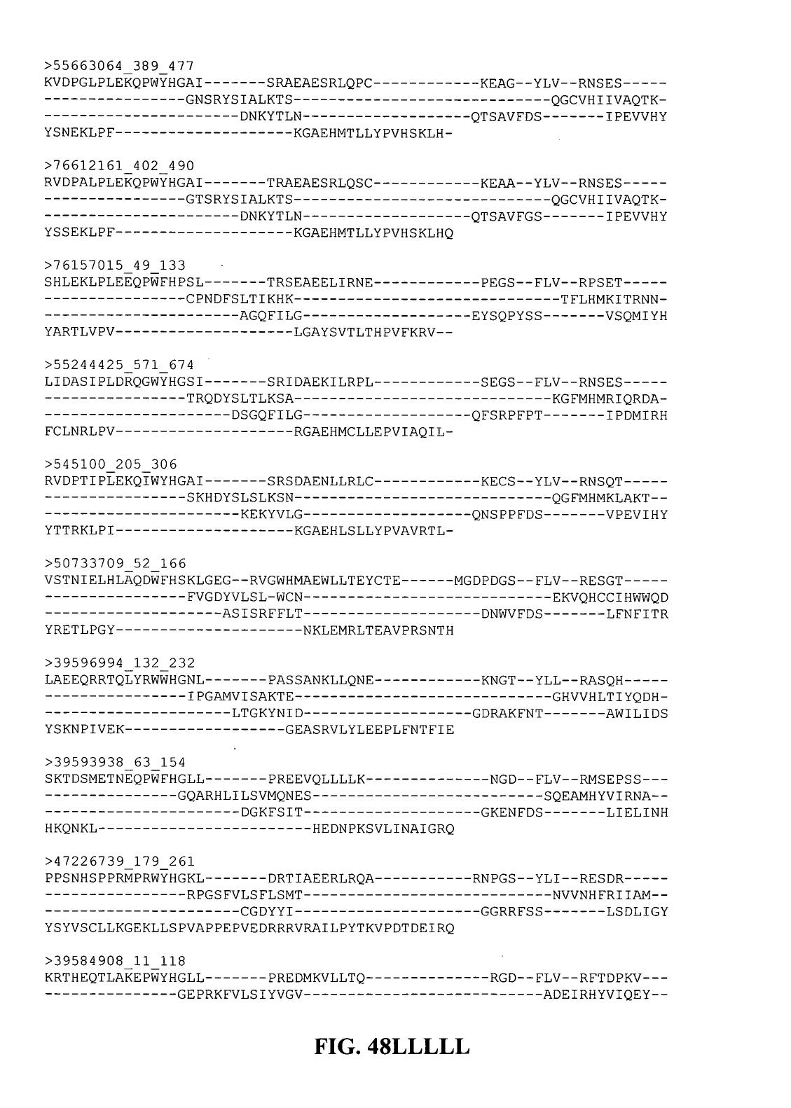

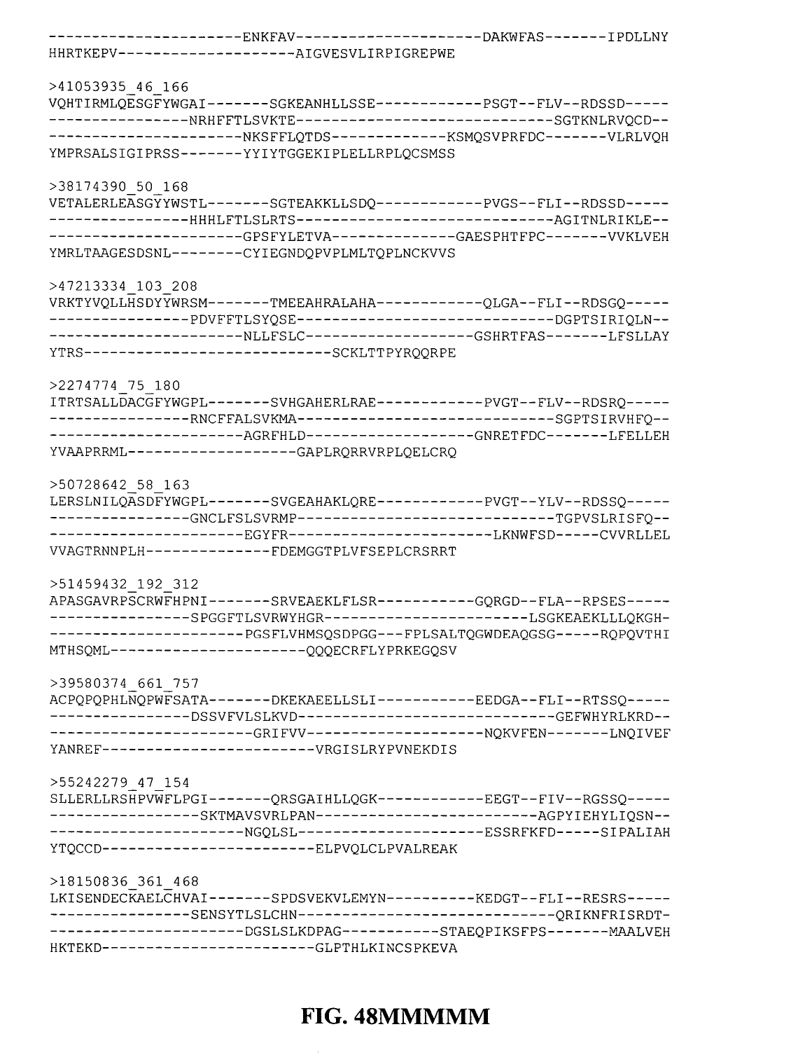

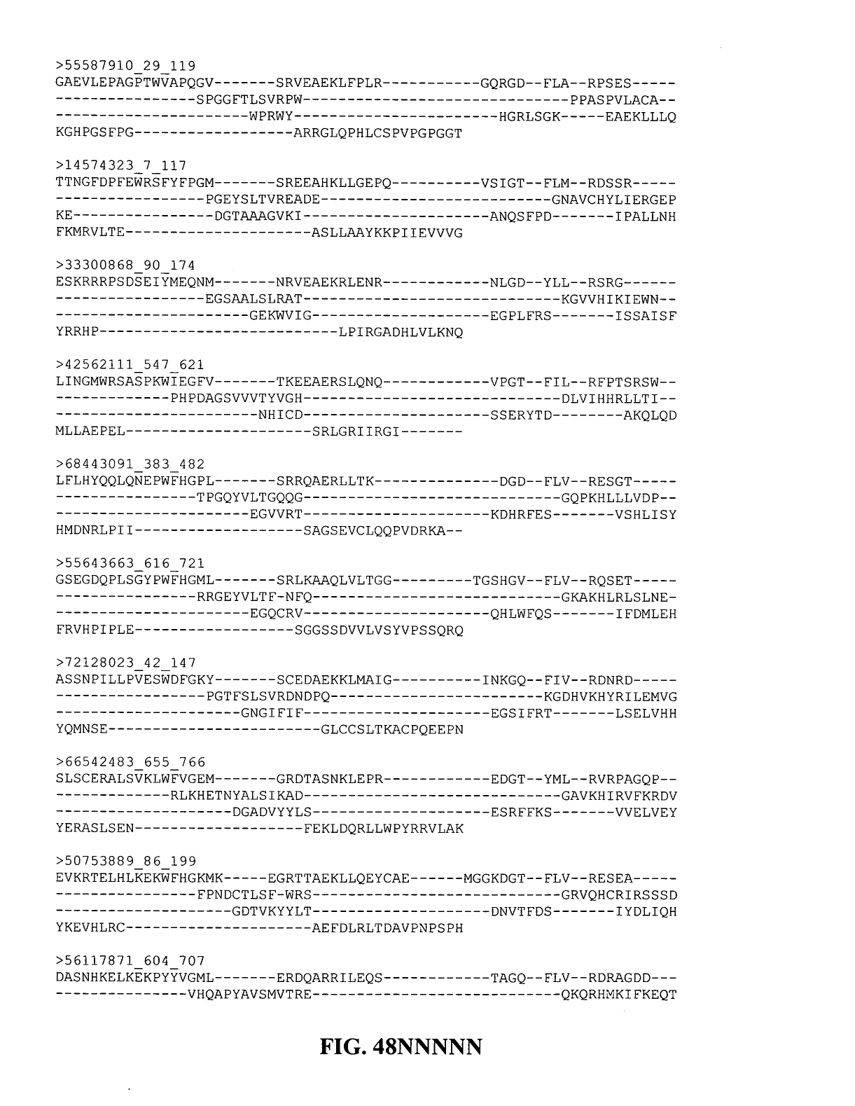

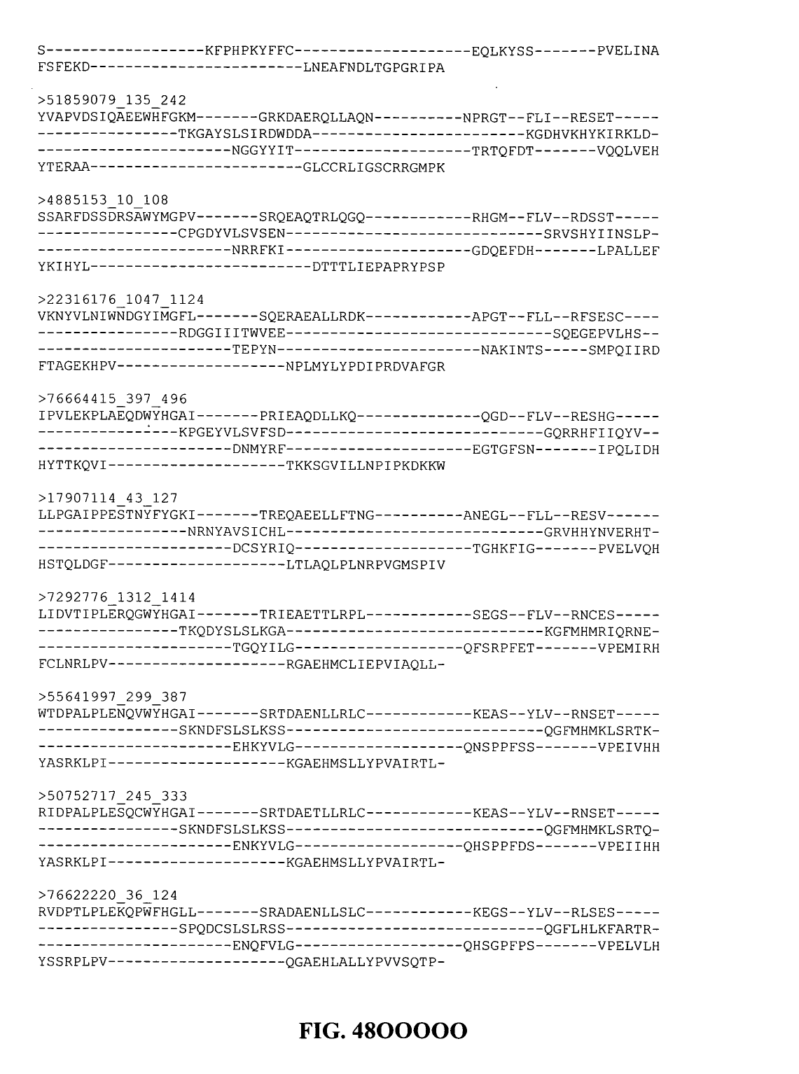

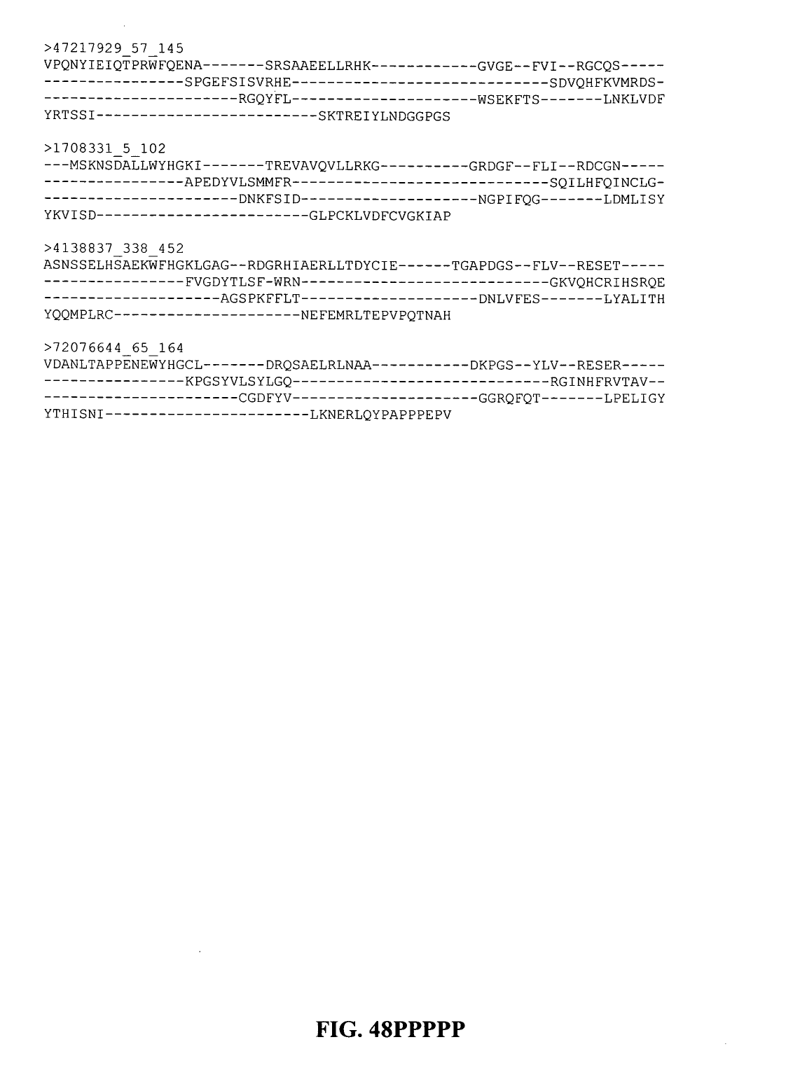

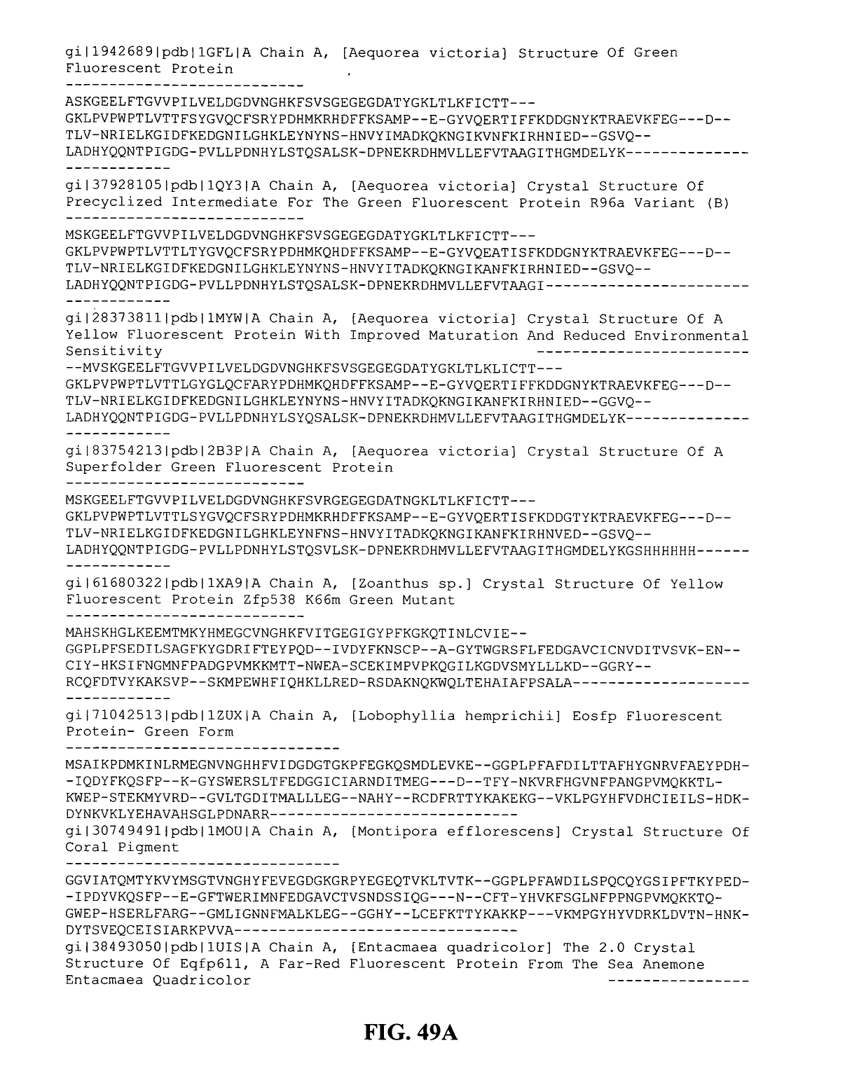

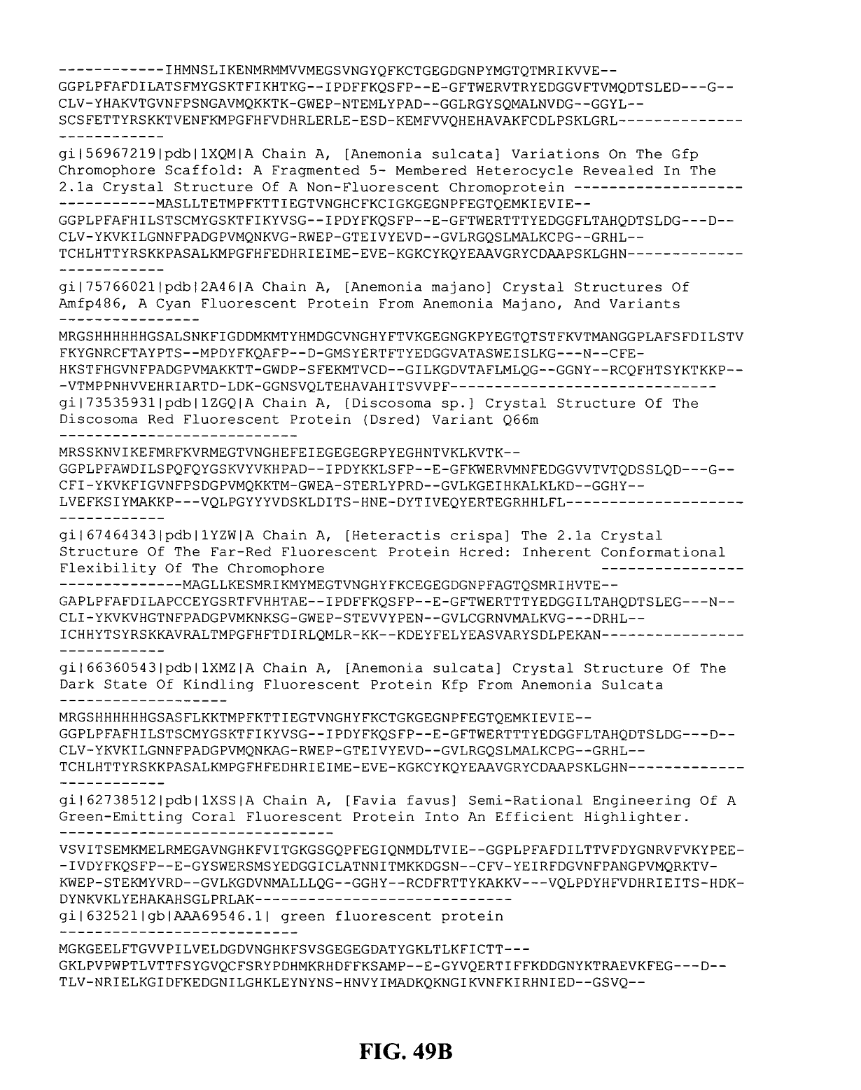

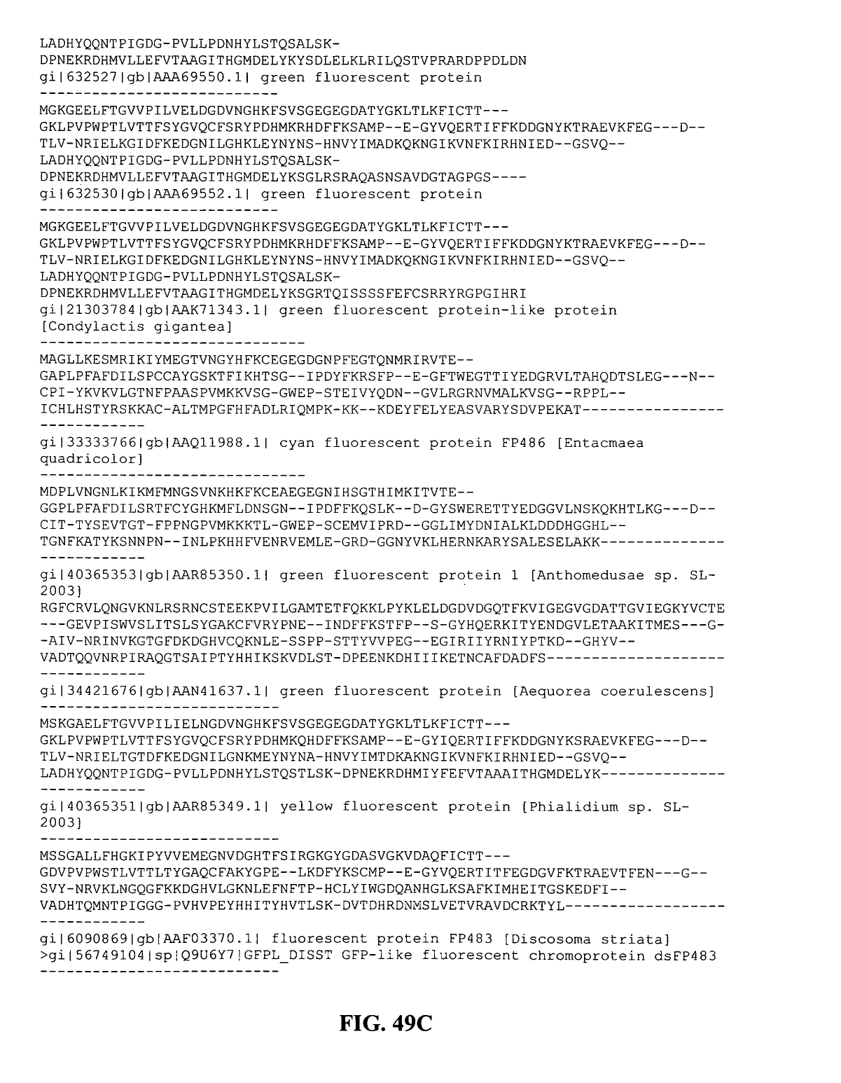

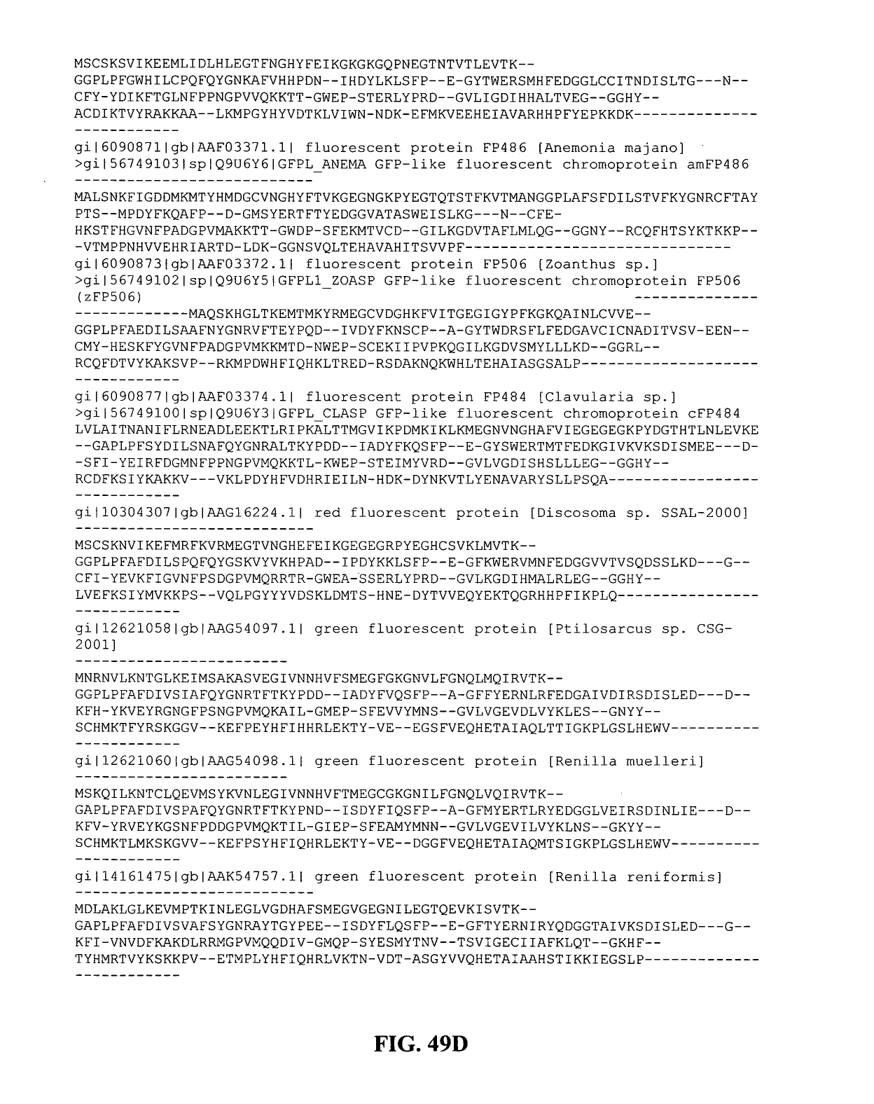

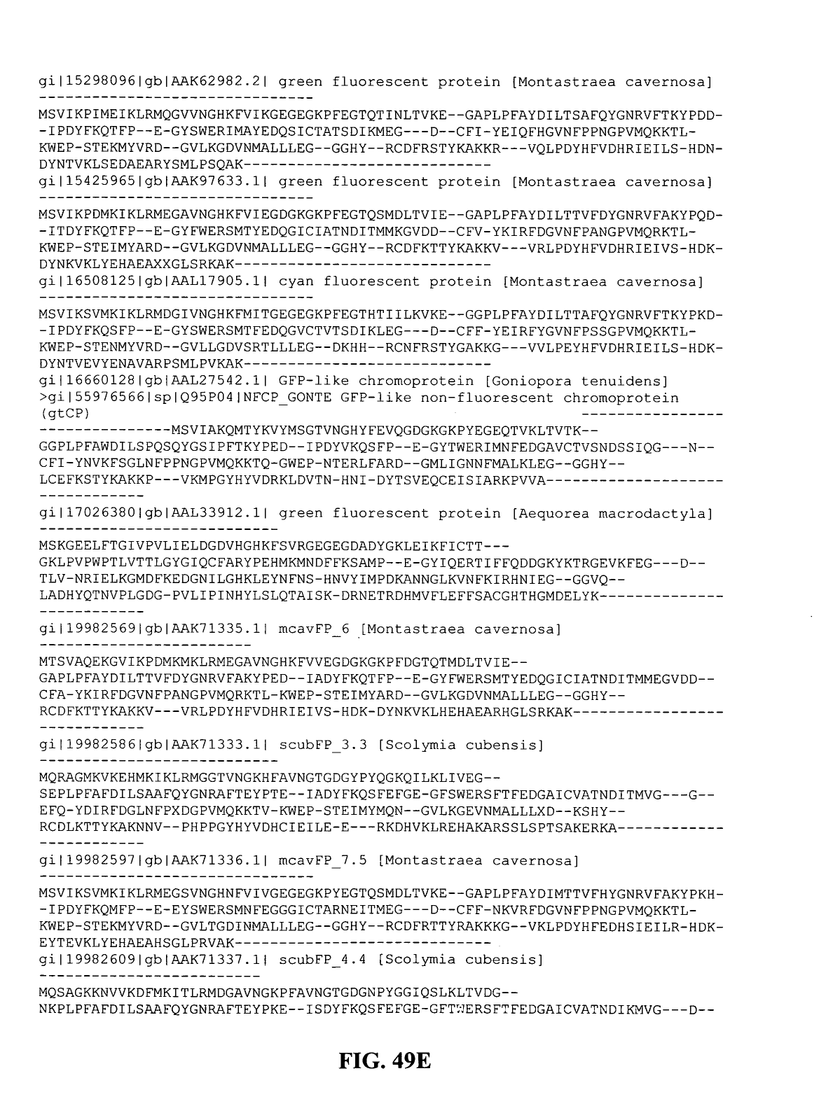

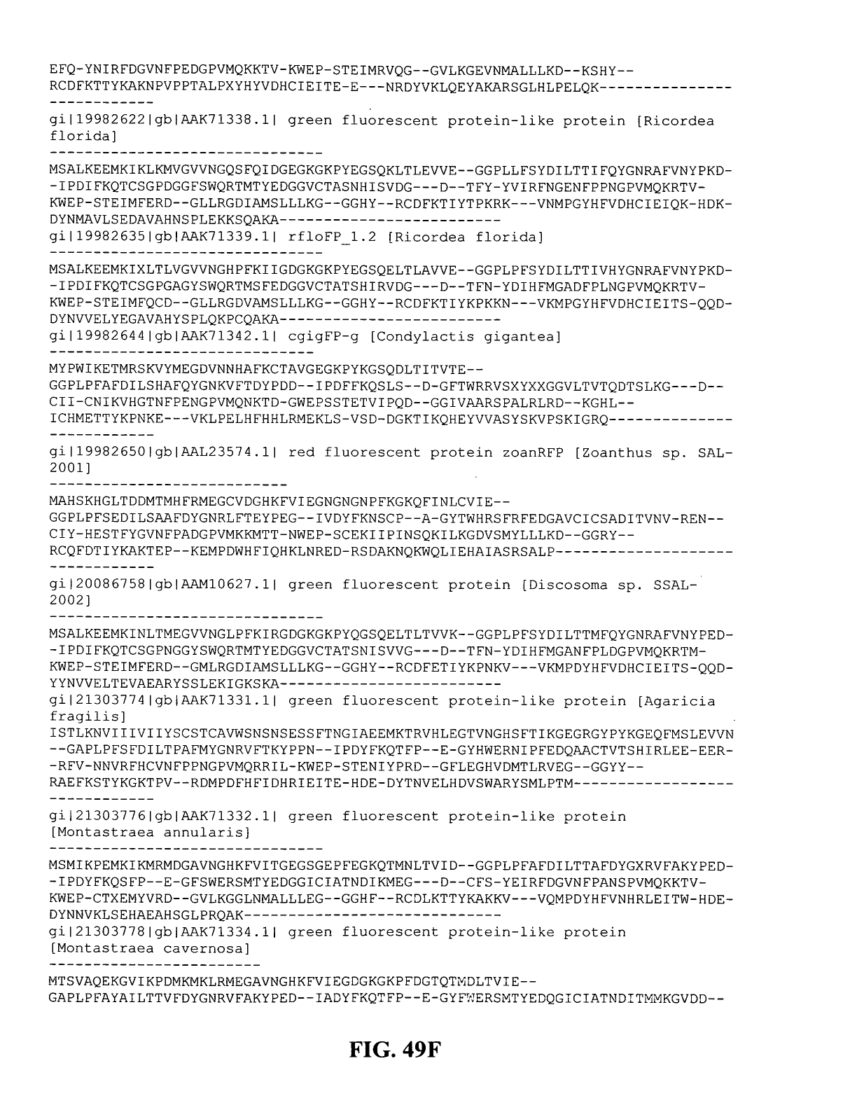

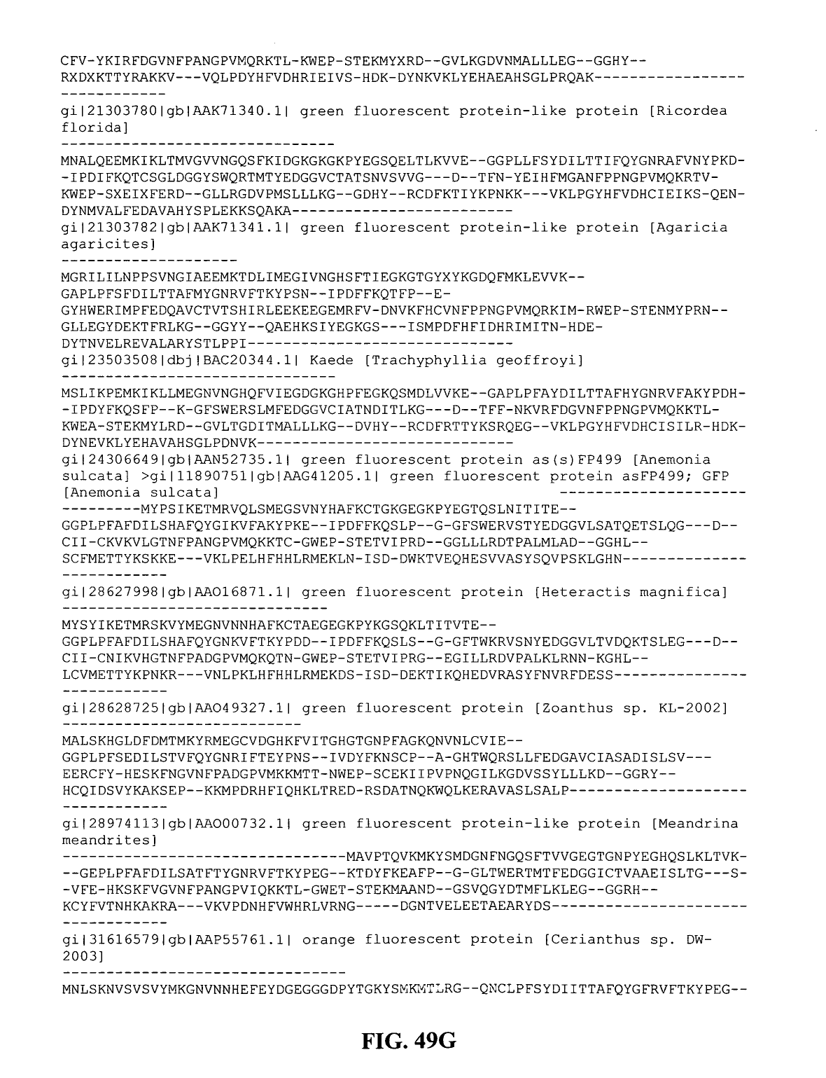

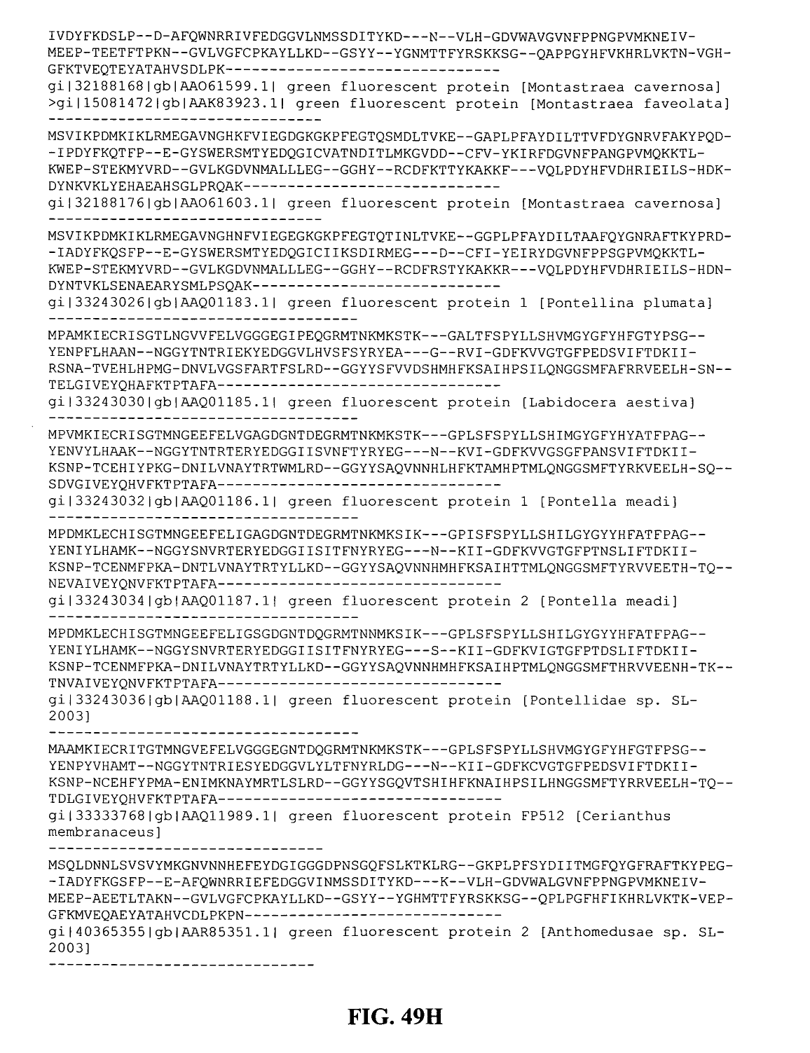

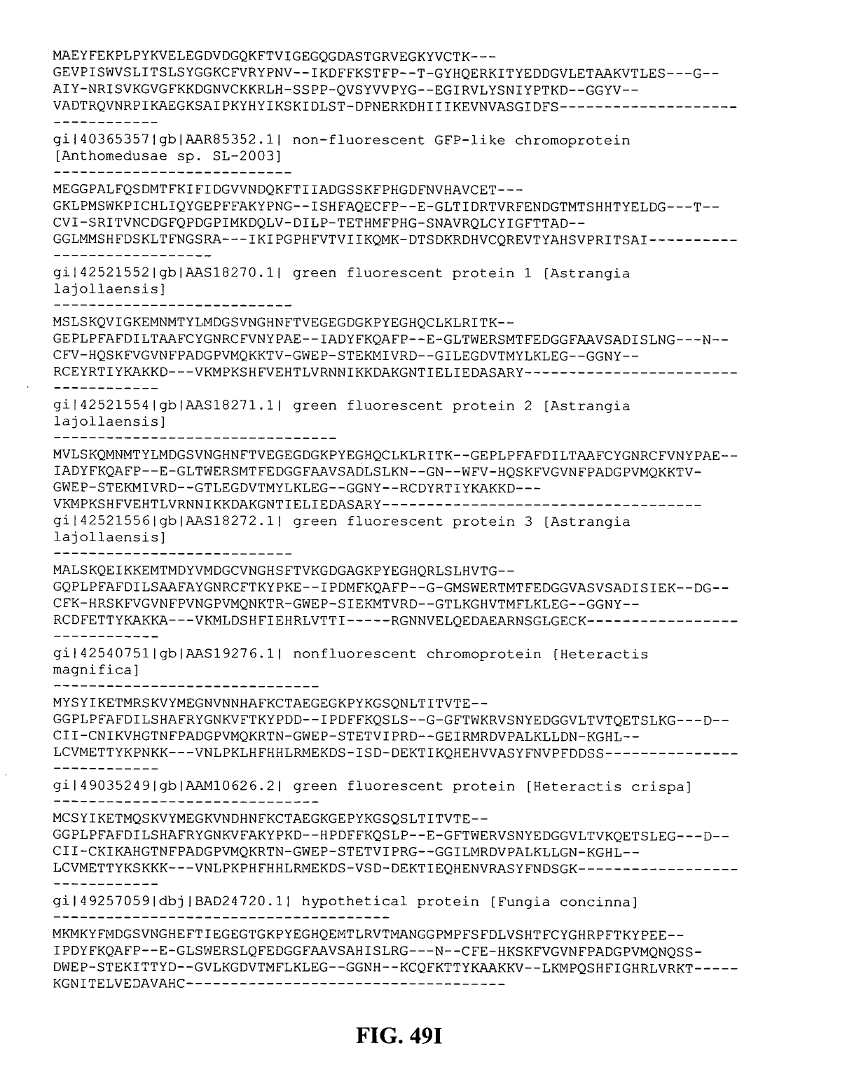

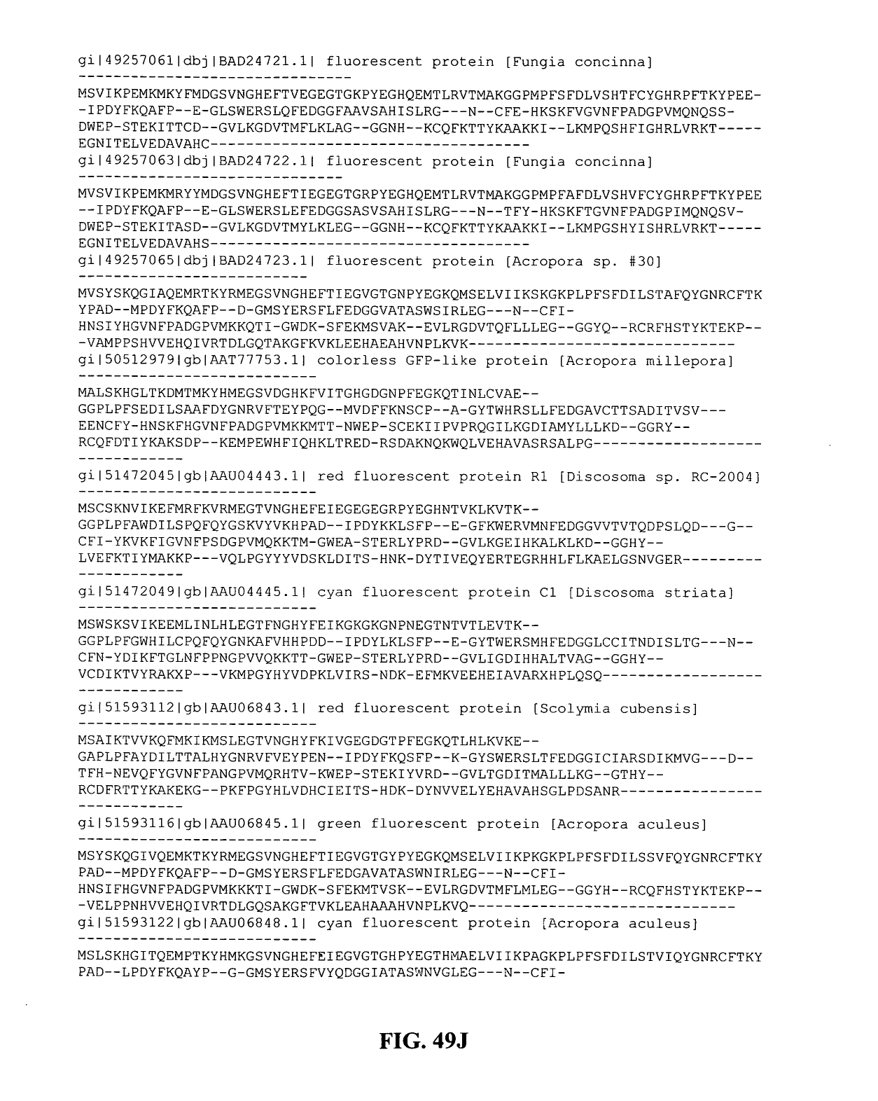

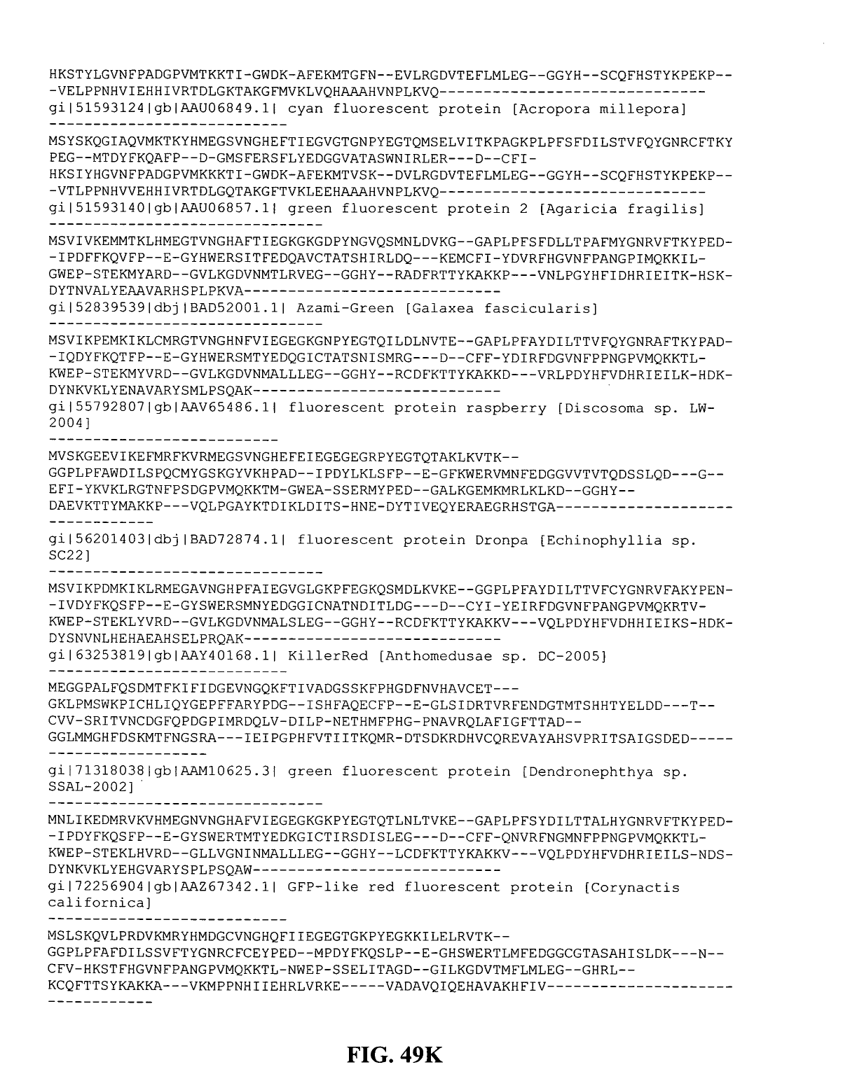

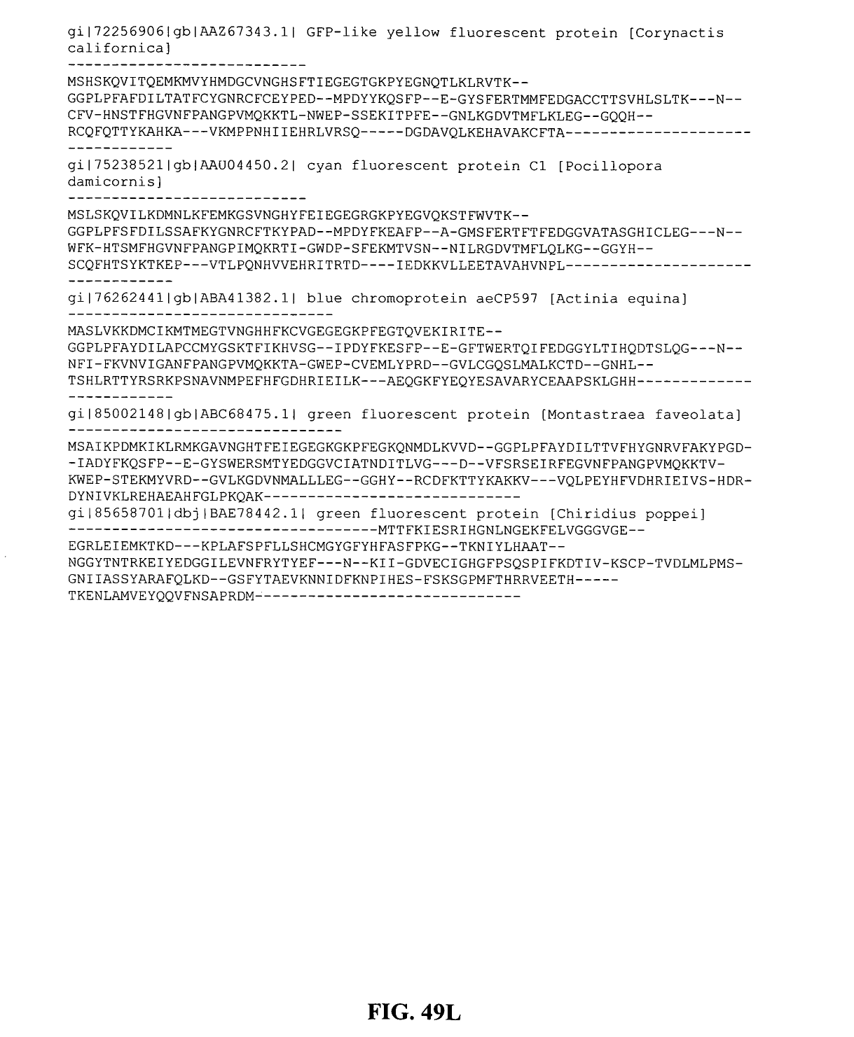

FIG. 42 depicts these two sets of positions mapped on a 3D structure of the 1 GFL and shows that the large network (blue) forms a largely contiguous set of residues that extends from both ends of the beta-barrel and interacts with the GFP chromophore (green sticks). The second, smaller network forms another set of packed residues at one end of the barrel (orange).

Consistency with know GFP mutations: sites identified in the two networks correlate with known mutations in fluorescent proteins such as GFP and RFP:

Jain and Ranganathan, PNAS 2004, 101(1) pp. 111-116. Positions around the chromophore found to have large energetic effects were 94, 96, 183, 203, 145, (which are part of the SCA network) and 69 and 222 (strongly coupled to network residues).

T203 is known to affect the absorbance spectrum, by stabilizing the protonated state and is mutated to Tyrosine in the yellow variant, YFP, and to Histidine in the photoactivatable variant developed by Jennifer Lippincott-Schwartz's lab. Patterson and Lippincott-Schwartz, Science 2002, 297 pp. 1873-1877.

Shaner, et al. Nature Biotechnology 2004, 22(12) pp. 1567-1572. Color tuning in RFP—we predict several sites that contribute significantly to the tuning in RFP mutants Orange, Banana and Honeydew (positions 84, 146, 148, 167, 179, 181, 203).

Campbell, et al. PNAS 2002, 99(12) pp. 7877-7882. Correlation with mutations in mRFP (a monomeric RFP variant): 42, 125, 167, 179, 181, 183, 203.

Wang, et al. PNAS 2004, 101(48) pp. 16745-16749. Correlation with a mutation in mRFP found to narrow the width of the emission spectrum (125).

In the Shaner, Campbell and Wang papers, the mutations that we identify were found by random mutagenesis. This suggests that our mutagenesis, targeting these sites, would be able to quickly access several of the same functional properties as found in the published studies. Furthermore, it shows that the residues identified by SCA have important functional impact, and suggests that the network positions that have not yet been studied will play a strong role in the tuning the function of these proteins. We expect that mutagenesis of SCA network residues in GFP will extend the spectral properties of existing natural fluorescent proteins.

Statistical Coupling Analysis in Proven Drug Targets

One embodiment of SCA was tested to determine whether it could be used to identify allosteric sites in proteins that may be suitable for targeted drug design. Two protein families were analyzed-caspases and glycogen phosphorylase—for which drugs have been identified that regulate activity by binding to known allosteric sites (away from the active site).

The Caspase family: Caspases are a family of dimeric cysteine proteases involved in programmed cell death (apoptosis) and inflammation. The version of SCA described above was performed on an alignment of 190 members of the caspase family, using the following commands:

[DEmat,DEvec]=dg(at);
perturbation_matrix=stat_fluc(at);
[cma,cmr]=global_sca(perturbation_matrix);

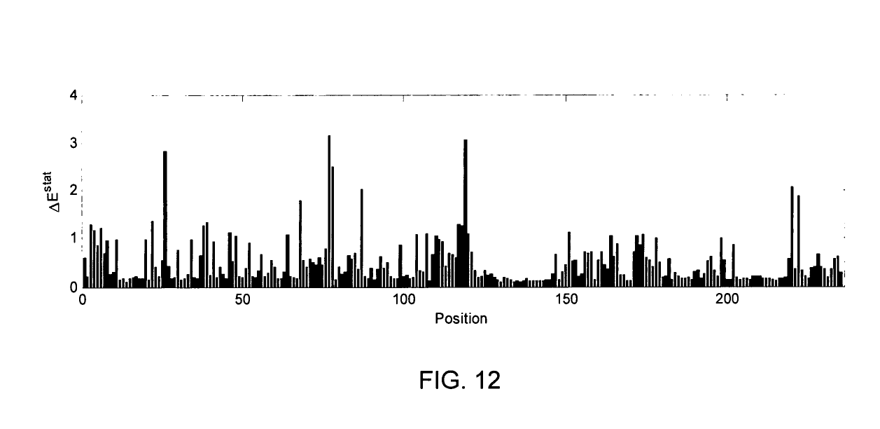

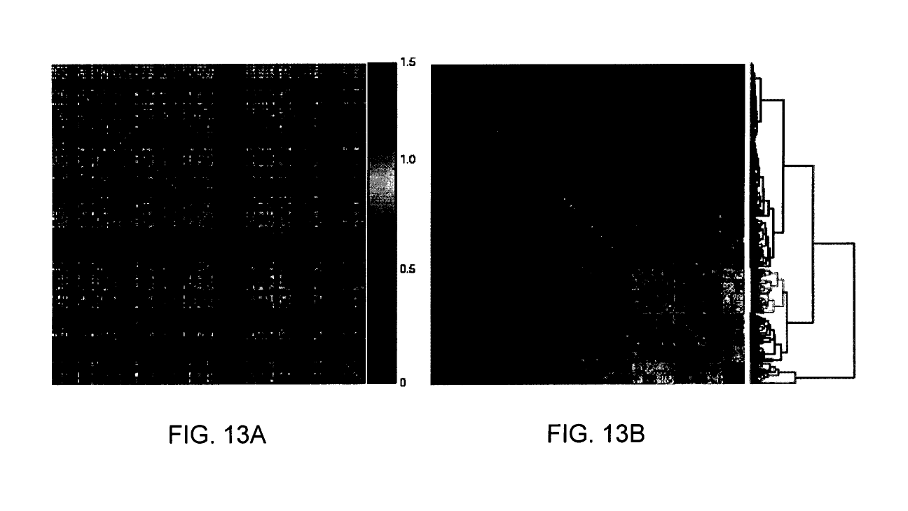

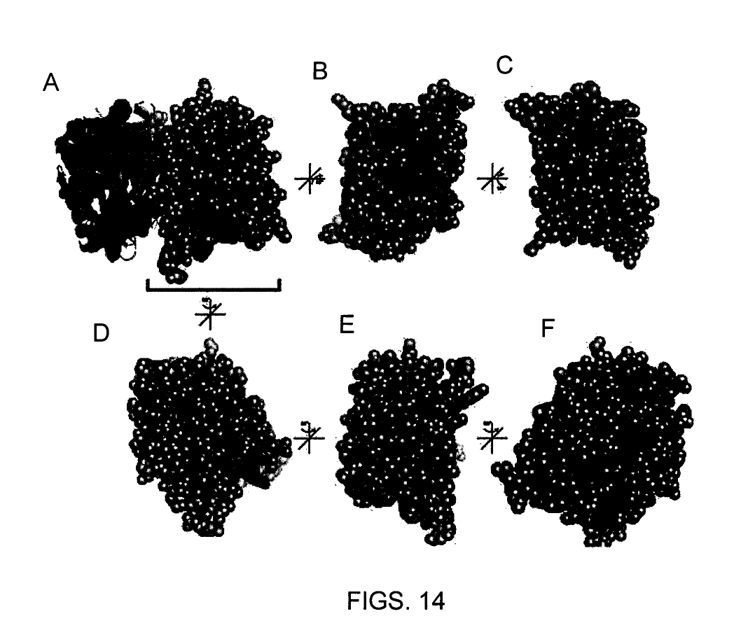

The conservation pattern for the caspase family shows several sites with very low conservation (FIG. 12), consistent with appropriate sequence diversity and alignment size. FIG. 13A shows the cmr matrix for the caspase family. FIG. 13B shows the results of performing the hierarchial clustering technique described above on the cmr matrix, and shows two dominant clusters. Mapped on the caspase structure (FIGS. 14A-F), the clusters show (as in other protein families) a contiguous network of interactions that links the active site to other functional surfaces (e.g., the dimer interface) through the core of the protein. Most of the network is buried in the core of the protein with only two solvent exposed surfaces comprising residues at the active site and residues at the dimer interface (FIG. 14B and FIG. 14D, respectively). Hardy et al. (2004) have discovered a ligand (DICA) that allosterically regulates proteolytic activity, which remarkably binds directly at the location in the dimer interface predicted by SCA to be coupled to the active site.

FIGS. 14A-14F show a crystal structure of human caspase-7 in complex with DICA and illustrate the stereochemistry of DICA recognition and correlation with the SCA predictions. This supports using SCA as a tool to discover potential allosteric sites for targeting drug design and discovery.

The Glycogen Phosphorylase family: Glycogen phosphorylase (glyp) is a critical enzyme in gluconeogenesis, converting glycogen into glucose-1-phosphate. glyp is allosterically regulated by a number of small molecules, including caffeine and AMP, as well as a class of indole-2-carboxamide inhibitors (CP-403,700) discovered by Rath et al. (2000) Applying SCA to this family demonstrates interaction of the network with all of these allosteric regulators.

One embodiment of SCA was conducted on an alignment of 152 glyp family members that showed good sequence diversity. The alignment was truncated to the sequence of human liver glycogen phosphorylase B for structural mapping, and the analysis was performed as described above, using the following commands:

[DEmat,DEvec]=dg(alignment);
perturbation_matrix = stat_fluc(alignment);
[cma,cmr]=global_sca(perturbation_matrix);

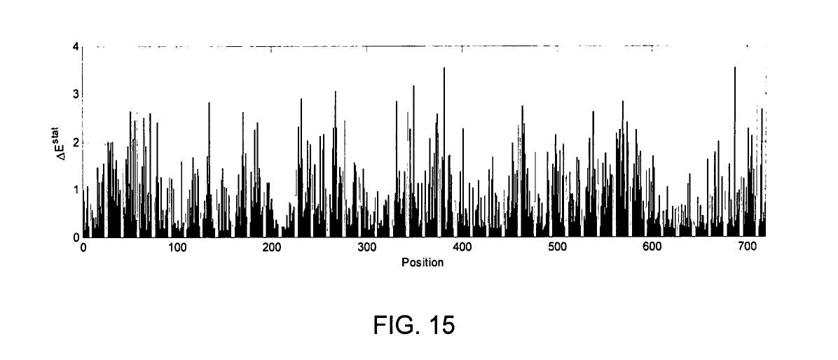

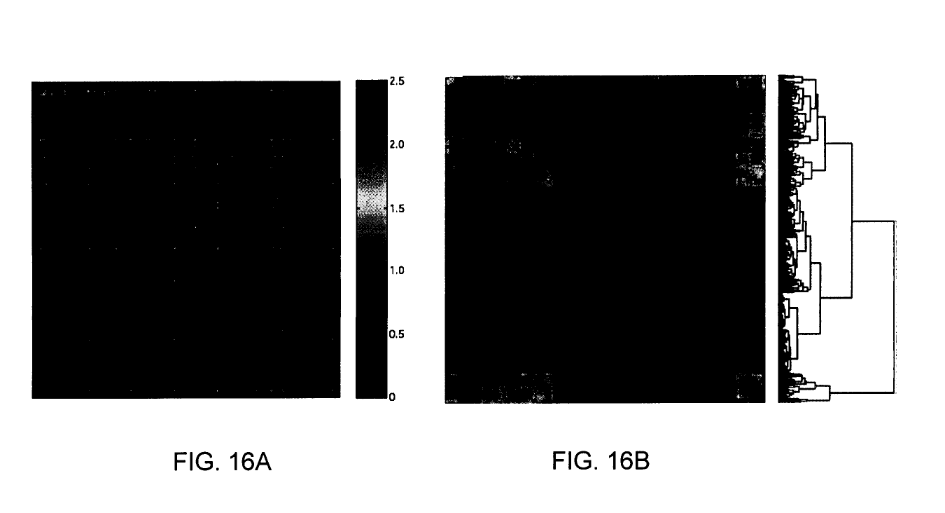

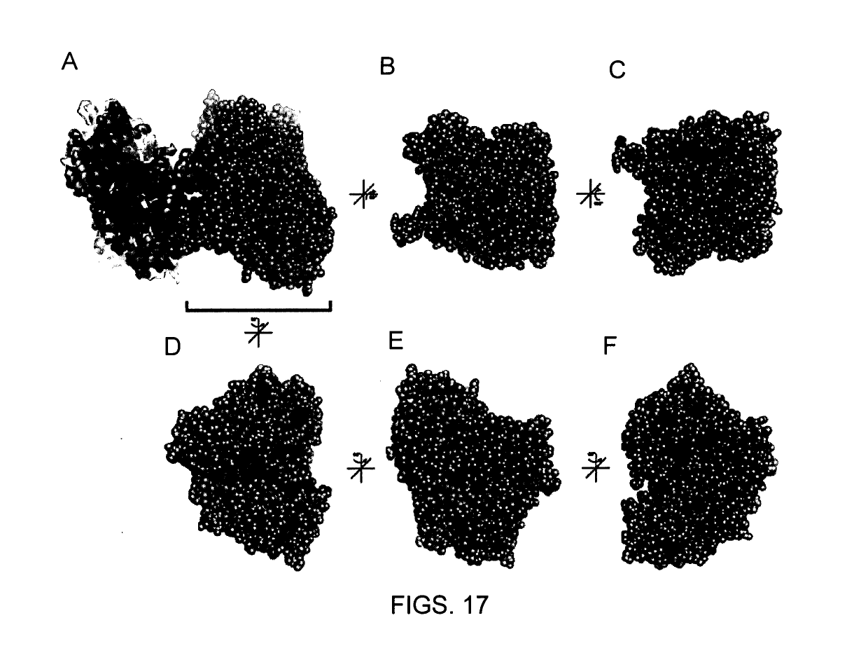

FIG. 15 shows the resulting conservation pattern. FIGS. 16A and 16B show the cmr matrix for the glyp family, both unclustered (FIG. 16A) and clustered (FIG. 16B). Clustering reveals two dominant clusters with similar patterns of coupling. Combining these two clusters and mapping on the structure of human glyp gives the results shown in FIGS. 17A-F. As in the caspases, the network is nearly fully buried, with solvent exposure limited to the active site and each surface site that directly contacts each of the allosteric ligands of glyp. The highly-limited solvent exposure of the SCA-identified sites (and yet the clear functional relevance of these mapping) highlights the value of SCA in focusing drug design and discovery processes around functionally-relevant surface sites.

It should be understood that although the version of SCA described above in detail is one suitable way to perform SCA on a family of biological sequences, it is clear from this disclosure that other ways exist that involve analyzing the coupling of fewer than all pairs of possible alignment positions, such as a version of SCA that analyzes the conserved co-variation between only one pair of biological position elements at one pair of biological sequence positions.

Designing Artificial Members of a Family of Biological Sequences

Some embodiments of the present methods include, in one respect, designing artificial biological sequences using the statistical conservation values, such as the ΔE_{i,x}^{stat} values that are calculated for the biological position elements of interest. One way of using those values is to use a dataset that is derived—at least in part—from them, such as one of the statistical coupling matrices described above. In this regard, the SCM that is used may, for example, comprise C^{ev} values calculated for all pairs of biological position elements at different positions in a given target alignment (whether, for example, in a cma matrix as C_{i,x,j,y}^{ev} values or in a cmr matrix as C_{i,j}^{ev} values), or C^{ev} values that are calculated based on as few as one pair of biological position elements at different positions.

The following description is one example of how to carry out designing artificial biological sequences using statistical conservation values:

First, the alignment, which may be characterized as a target alignment and has M biological sequences that are functionally organized in M rows and N columns, may be altered to yield an altered alignment that retains M biological sequences in M rows and N columns. The alteration may comprise introducing sequence diversity into the target alignment by shuffling (e.g., randomly) at least two biological position elements within one or more positions (columns) of the target alignment. Throughout this disclosure, alignment positions and sequence positions of alignments mean the same thing. When done randomly, the shuffling process may be characterized as randomizing an alignment. The alteration process may be characterized as diversifying an alignment.

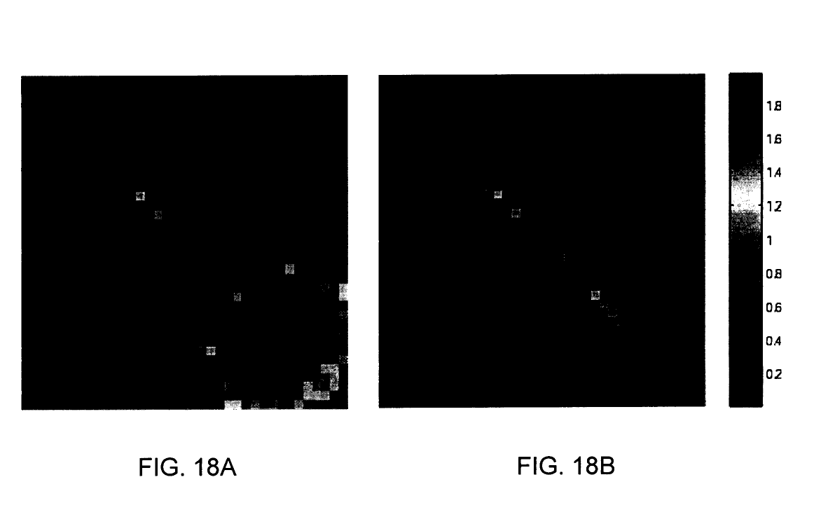

When carried out for each alignment position independently, such shuffling results in an altered (e.g., randomized) alignment having the same size as the target (or starting) alignment. The altered alignment has the same sequence position conservation pattern (because no changes are made to the position-specific frequencies of biological position elements). If sufficiently shuffled, the statistical couplings between pairs of biological position elements at different positions will be substantially or completely destroyed. FIGS. 18A and 18B show a cmr matrix both before and after shuffling.

After altering the alignment, additional shuffling of biological position elements within each alignment column may occur, but with a target of substantially reproducing the SCM of the target alignment. One way to achieve this target is to use an optimization algorithm, such as a simulated annealing algorithm, one type of which is a Metropolis-Monte Carlo algorithm. Such an algorithm follows an iterative process, one example of which may be carried using the program SCA-MC.c (implemented in C++ and included as the file SCAMC.txt in the computer program listing appendix submitted on CD with this application). The SCA-MC.c program performs the following algorithm:

a) randomizing the alignment to yield a randomized alignment that retains M biological sequences in M rows and N columns, the randomizing comprising randomly selecting at least two rows and one column of the target alignment and swapping the two biological position elements present at the two selected positions of the target alignment to yield the randomized alignment, which may be characterized as alignment₀ (the 0^{th} iteration alignment);

b) obtaining (e.g., calculating) an SCM₀ for alignment₀, where the SCM₀ comprises a cmr matrix;

c) swapping two biological position elements within one column of the alignment₀ to yield an alignmentₙ, where each swapping comprises an iteration, and n comprises the number of iterations;

d) obtaining an SCMₙ for the alignments, where the SCMₙ comprises a cmr matrix;

e) evaluating a parameter called the system energy at the n^{th} iteration (eₙ), where the evaluating comprises obtaining (e.g., calculating) a system energy value eₙ, where eₙ=|SCMₙ−SCM_{target}|, eₙ is a scalar value that quantitatively represents how different the alignment at the n^{th} iteration is from the target alignment, and SCM_{target} is a cmr matrix calculated for the target matrix;

f) calculating the difference in system energy Δe (also characterizable as a scalar system energy value) between the n^{th} iteration and the n^{th} iteration, where Δe=eₙ−eₙ₋₁; and

g) determining whether to accept a swap from c) for a given iteration, where the determining is based on the following criteria:
i) if Δe≦0, accepting the swap (because the alignment at iteration n draws closer in terms of system energy to the target alignment than the n−1^{th} iteration); and
ii) if Δe>0, accepting the swap with a Boltzmann-weight probability

$$( {P}_{accept} = {ⅇ}^{- \frac{Δ e}{β}} )$$

(which is one form of a non-zero probability), where β is a statistical equivalent to temperature in a physical system and controls the likelihood of accepting swaps that diverge the simulation from the SCM_{target}. The parameter β is initially set high enough to keep the alignment as distant from the SCM_{target} as the SCM₀, and then exponentially reduced until convergence is substantially reached (see f) above).

The parameter β is reduced during iterations in e) when a preset number of swaps is accepted such that β_{new}=0.9β_{old}. The preset number of swaps in the SCA-MC code is 10-fold coverage of the alignment. For a M×N alignment, this means 5×M×N swaps. The simulation completes, in this embodiment, when a preset number of swaps is not accepted upon three sequential attempts at 100-fold coverage of the alignment. For a M×N alignment, this means 50×M×N swaps.

In the beginning of the simulation represented in the embodiment coded in SCA-MC, β is high, allowing many “unfavorable” swaps that increase the system energy to a maximal level. As β is lowered, the selection for swaps becomes increasingly stringent, causing the n^{th} SCM to become increasingly similar to the target SCM.

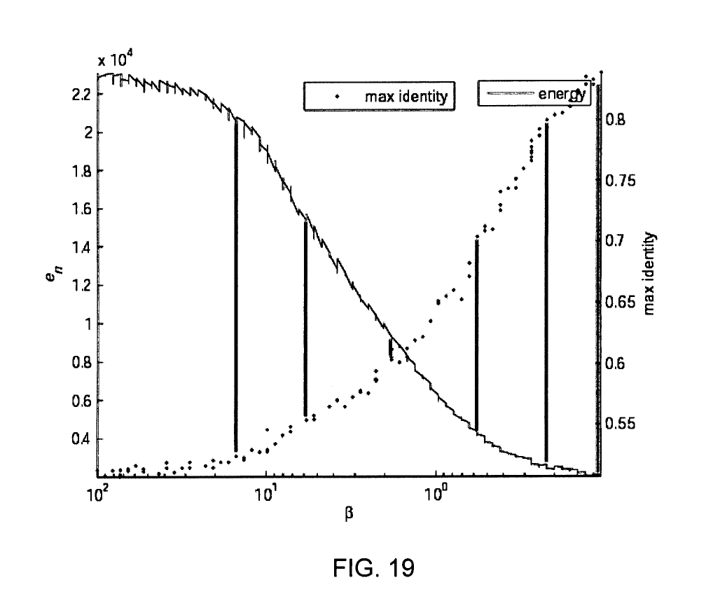

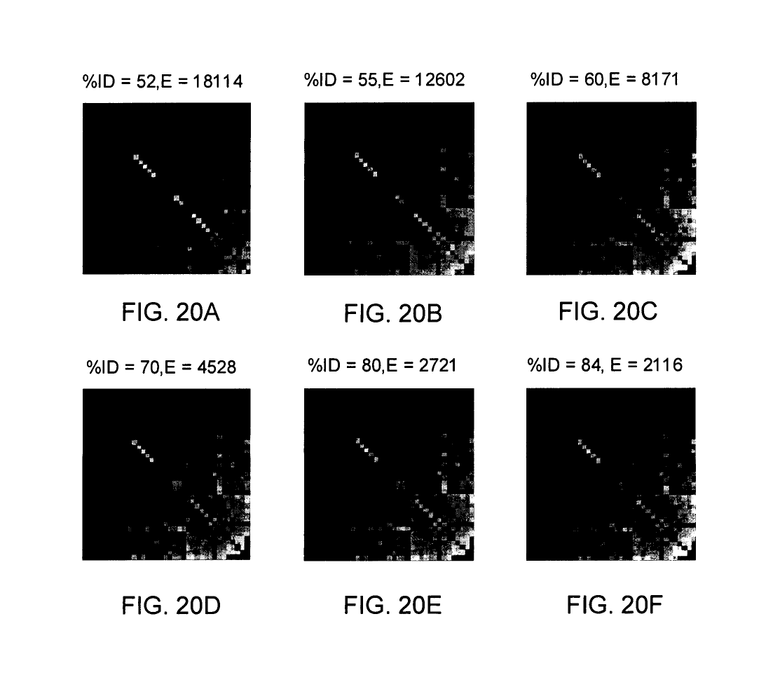

For the WW domain family, the energy trajectory for one run of this coded simulation is graphed in FIG. 19, and the cmr matrices corresponding to several points along the trajectory are shown in FIG. 20. As the energy converges on a minimum value, the sequences become more similar to natural WW domains; as a result, the maximum (or top-hit) pairwise identity between the artificial sequences and natural WW domains increases to a maximum value. The “top-hit” identity of an artificial sequence can be assessed as follows. Assume the natural alignment has 10 protein sequences. Compare an artificial sequence to each of the 10 natural sequences. Any position that has the same amino acid in the artificial sequence as in a given natural sequence counts as an “identity.” The percentage identity is the number of identities divided by the number of positions in the sequence/alignment. Comparing the artificial sequence to the 10 natural sequences gives 10 values for the percentage identity. The highest value among these is the “top-hit identity” for that artificial sequence. It reveals how similar the artificial sequence is to any natural sequence in the alignment. For instance, if the artificial sequence is identical to one of the natural sequences, then the top-hit identity would be 1 (or 100%).

In the file SCA-MC.c that appears below, references to “DG” and “DDG” variables are to be read as “DE” and “DDE”, which are used throughout the code provided elsewhere in this description. The stdio.h, math.h and tim.h files referenced in SCA-MC.c are part of the Standard C library, and need not be recited.

The alloc_swl.h, align.h and gamma.h files that are used with the SCA-MC.c file provided below each appear at the end of the description but before the claims under the general heading “COMPUTER PROGRAM LISTINGS.”

Designing Artificial Members of a Family of Biological Sequences Using a Subset of the SCM

An alternative technique to the one described above for designing artificial biological sequences using statistical conservation values involves, broadly, eliminating information from the chosen SCM during application of the optimization algorithm (such as the Metropolis-Monte Carlo simulated annealing algorithm described above), such that the optimization algorithm runs on a subset of the chosen, or target, SCM. It has been discovered that complete convergence of the Metropolis-Monte Carlo trajectory (as performed using SCA-MC.c) on a full SCM yields a set of artificial sequences with high identities to the initial set of natural sequences. One approach to designing artificial sequences with lower identities is to eliminate data (such as data that is evolutionarily unimportant) from the SCM while still retaining the information useful to designing folded, functional artificial sequences. The data elimination may be logical rather than actual in that it may involve adapting the algorithm to operate only on a subset of the SCM (e.g., by masking off the “eliminated” data).

The manner in which artificial sequences may be designed using this “mask” approach can be accomplished using the relevant computer programs provided in this disclosure in the manner described above and by performing the optimization algorithm contained in SCA-MC-2-mask-AP.c (implemented in C++ and included as the file SCAMCMASK.txt in the computer program listing appendix submitted on CD with this application). This approach is the same as the approach described above in Designing Artificial Members of a Family of Biological Sequences except that (1) the step of evaluating the system energy is performed on a subset of the SCM_{target} and therefore also on a subset of the SCMₙ; (2) the preset number of swaps is equal to M (the number of sequences in the alignment)×(M−1)×N (the number of positions in the alignment); (3) the test for equilibration is based on different criteria; and 4) a finer temperature step size is used—the temperature decreases by 0.95 each round instead of by 0.9. The input file stdlib.h referenced in SCA-MC-2-mask-AP.c is it part of the Standard C library and need not be recited.

There are many different ways to eliminate information from the target SCM in order to allow for determination (e.g., calculation) of the system energy on a subset of that SCM. For example, any of the following techniques may be employed:

1) backing up along the energy trajectory. As the Monte Carlo algorithm converges, the energy drops and the sequence identity rises. If alignments from points along the convergence trajectory are taken, sequences with relatively low energy (the matrix is mostly converged) but with relatively low identity (the sequences are still different) can be found.

2) significance mask (or “sigma mask”). One way to disregard some elements of the SCM that may be insignificant is to create a significance cutoff, or a significance criteria. For example, any C^{ev} coupling values that are less than two standard deviations below the mean could be left out of the calculation. The remaining entries that have high C^{ev} values may contribute almost all of the information necessary to recreate the matrix, but give rise to lower sequence identities.

3) stripe mask. Another approach involves identifying the dominant co-evolving network(s) using, e.g., clustering or ICA, and using only the C^{ev} values involving these residues. Taking this approach defines a “stripe” of coupling across the SCM that contributes to the energy calculation. Other entries in the SCM are disregarded by the Monte Carlo algorithm. As with the significance mask, the result is that most of the energy is recreated by the Monte Carlo algorithm, but the resulting sequences have a lower identity relative to the natural sequences of the original alignment.

The sigma mask described above was performed using the SCA-MC-2-mask-AP.c code on three different protein families: SH3 domains, Dihydrofolate Reductase, and SH2 domains.

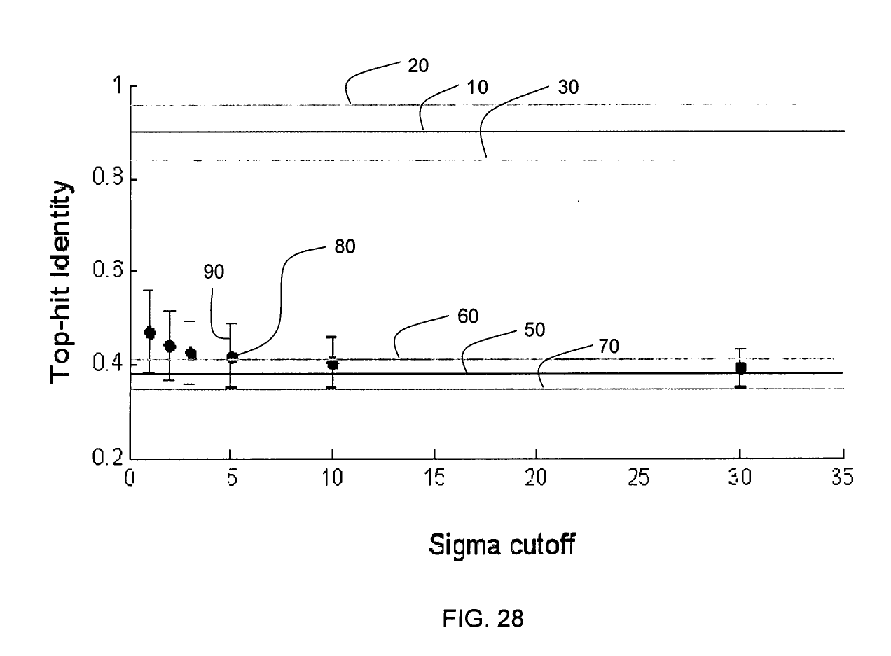

In FIG. 28, which concerns the SH3 domain, line 10 is the mean top-hit identity between artificial SH3 sequences created using the version of the optimization algorithm described above that did not involve the use of a mask (which includes SCA-MC.c) and the sequences of the natural SH3 alignment. Line 20 represents +1 standard deviation from mean 10, and line 30 represents −1 standard deviation from mean 10. The points designated by element number 80 (only one of the six points is labeled, but the label applies to each) represent the top hit identity between artificial SH3 sequences created using the SCA-MC-2-mask-AP.c code where sigma cutoff masks of 1, 2, 3, 5, 10 and 30 standard deviations above the mean conserved co-evolution score (C^{ev}) of the entire SCM were used (which scores were calculated using the techniques described above), and the sequences of the natural SH3 alignment. The errorbars identified by element number 90 (again, the label applies to each bounded vertical bar) span+/−1 standard deviation from the mean top-hit identity produced by each sigma cutoff mask run. Line 50 represents the mean top-hit identity between the sequences in the randomized alignment (in which the biological position elements of the natural alignment were shuffled to maintain the conservation pattern but destroy the coupling between sites), which can be created using either the SCA-MC.c program or the SCA-MC-2-mask-AP.c program, and the sequences of the natural SH3 alignment. Line 60 represents +1 standard deviation from mean 50, and line 70 represents −1 standard deviation from mean 50.

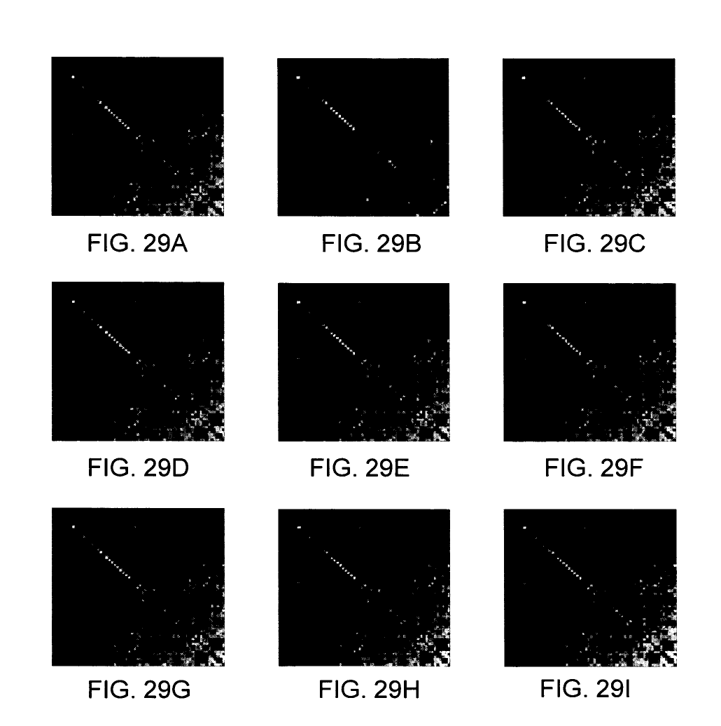

FIG. 29A is a cmr matrix of the natural SH3 alignment. FIG. 29B is a cmr matrix of the randomized alignment, which was created using the version of the optimization algorithm described above that lacks a mask and includes SCA-MC.c, but which can also be created using a version of the algorithm that includes a mask (such as the version that includes SCA-MC-2-mask-AP.c). FIG. 29C is a cmr matrix of artificial SH3 sequences created using the version of the optimization algorithm described above that lacks a mask and includes SCA-MC.c, but which could have been created using a version that includes a mask. FIGS. 29D-I are each a cmr matrix of the artificial SH3 sequences created using the version of SCA described above that includes a mask (which includes SCA-MC-2-mask-AP.c), where the mask was set such that the significance cutoff was chosen as one of the standard deviations above the mean conserved co-evolution score (C^{ev}) of the entire SCM (those standard deviations are 1, 2, 3, 5, 10 and 30).

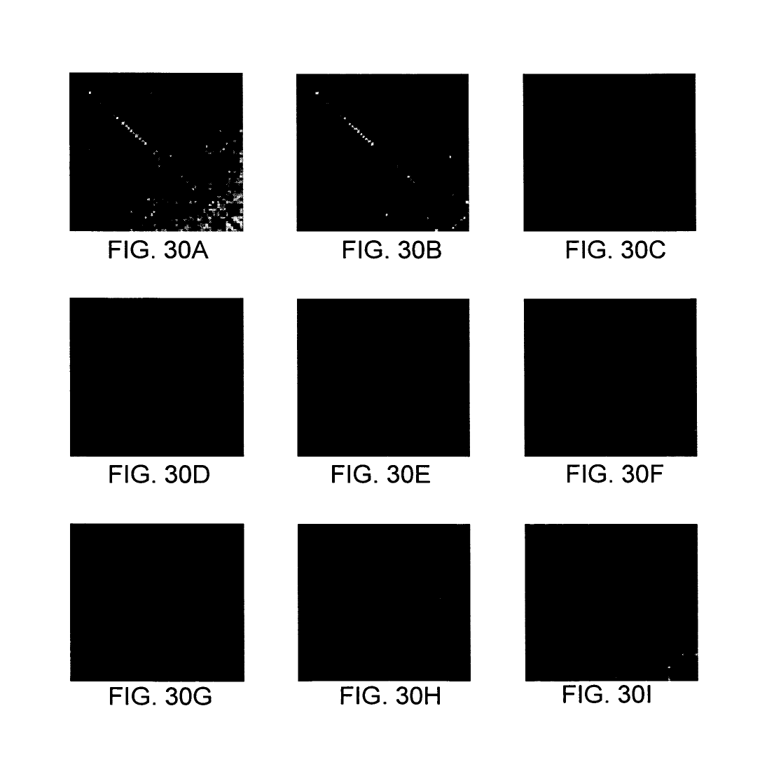

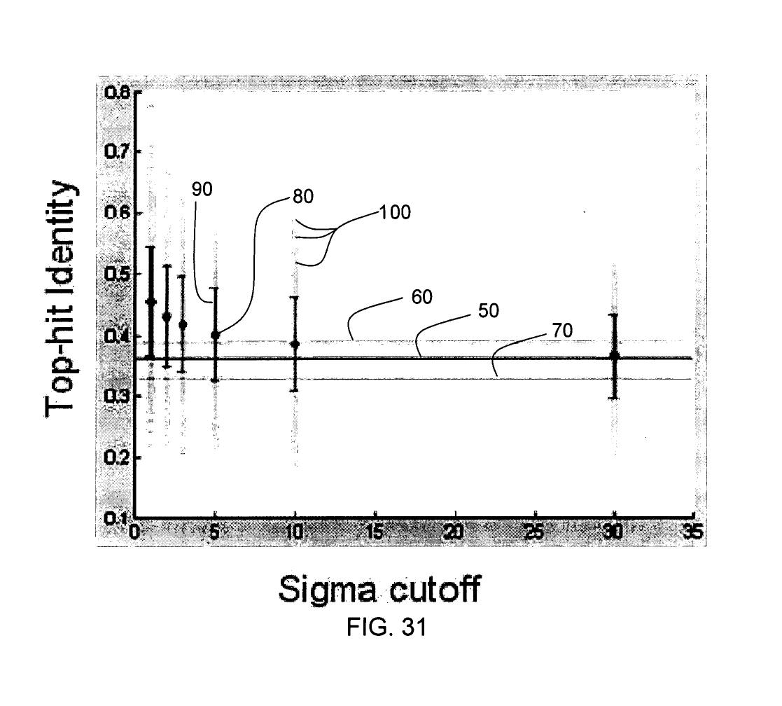

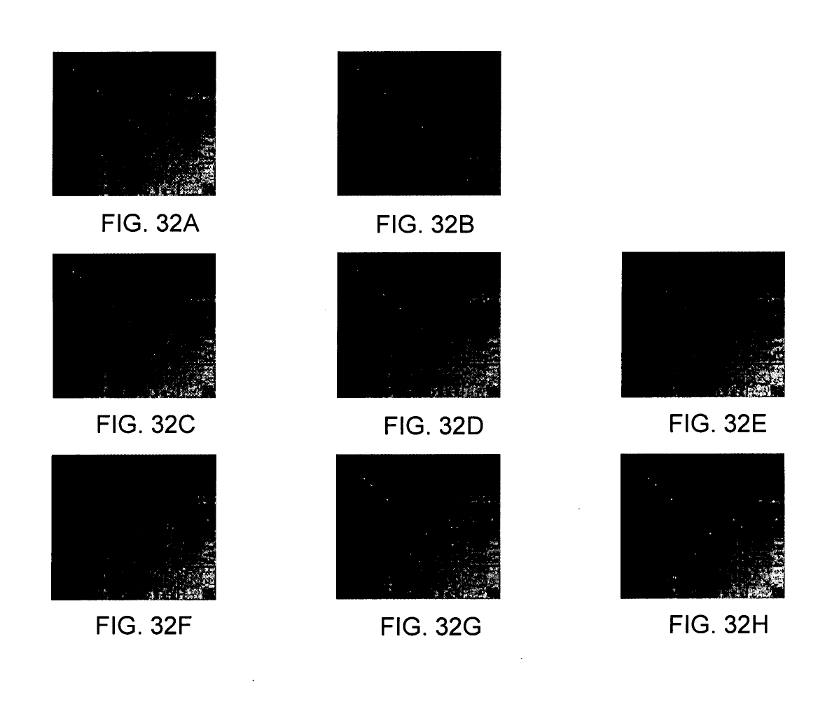

FIGS. 30A-I are included to illustrate the effectiveness of the masking techniques employed. FIG. 30A shows the cmr matrix of the natural SH3 alignment again. FIG. 30B is a difference matrix that was calculated between the cmr matrix of FIG. 30A and the cmr matrix shown in FIG. 29B. FIGS. 30C-I are difference matrices, respectively, between the cmr matrix shown in FIG. 30A and those shown in FIGS. 29C-I. Each difference matrix is the absolute value of the difference between the cmr matrix of the natural SH3 alignment and the respective sigma cutoff matrix.

FIG. 31 shows comparable values to those in FIG. 28 that were determined using an alignment of natural Dihydrofolate Reductase sequences. The points (which blend together) labeled with element number 100 (only one of the six groups of points is labeled, but the label applies to each) represent the individual top-hit identity values between each artificial sequence and those of the natural alignment.

FIG. 32A is a cmr matrix of the natural Dihydrofolate Reductase alignment. FIG. 32B is a cmr matrix of the randomized alignment, which was created using the version of the optimization algorithm described above that lacks a mask and includes SCA-MC.c, but which can also be created using a version the algorithm that includes a mask (such as the version that includes SCA-MC-2-mask-AP.c). FIGS. 32C-H are each a cmr matrix of the artificial Dihydrofolate Reductase sequences created using the version of SCA described above that includes a mask (which includes SCA-MC-2-mask-AP.c), where the mask was set such that the significance cutoff was chosen as one of the standard deviations above the mean conserved co-evolution score (C^{ev}) of the entire SCM (those standard deviations are 1, 2, 3, 5, 10 and 30).

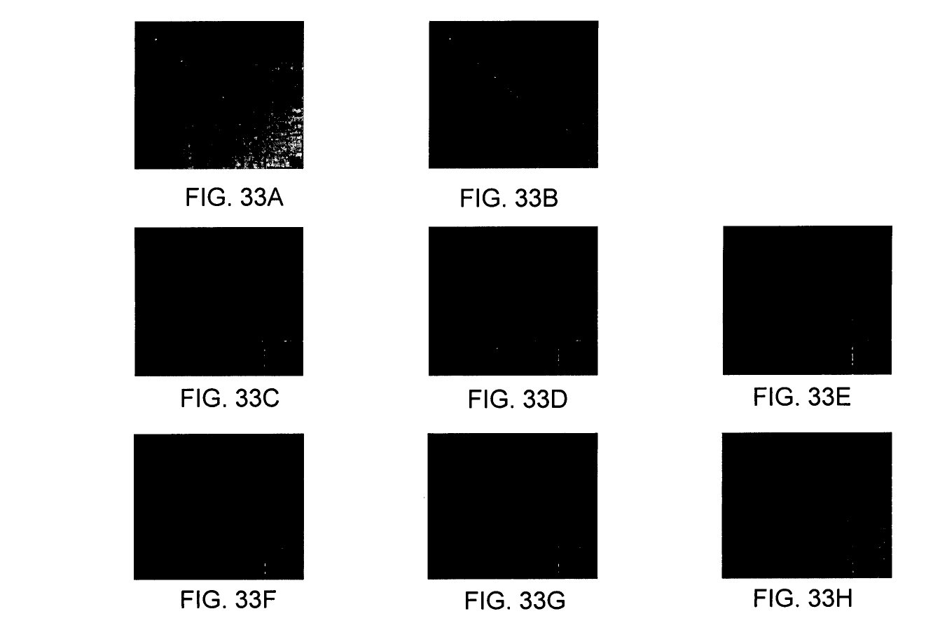

FIGS. 33A-H are included to illustrate the effectiveness of the masking techniques employed. FIG. 33A shows the cmr matrix of the natural Dihydrofolate Reductase alignment again. FIG. 33B is a difference matrix that was calculated between the cmr matrix of FIG. 33A and the cmr matrix shown in FIG. 32B. FIGS. 33C-H are difference matrices, respectively, between the cmr matrix shown in FIG. 33A and those shown in FIGS. 32C-H.

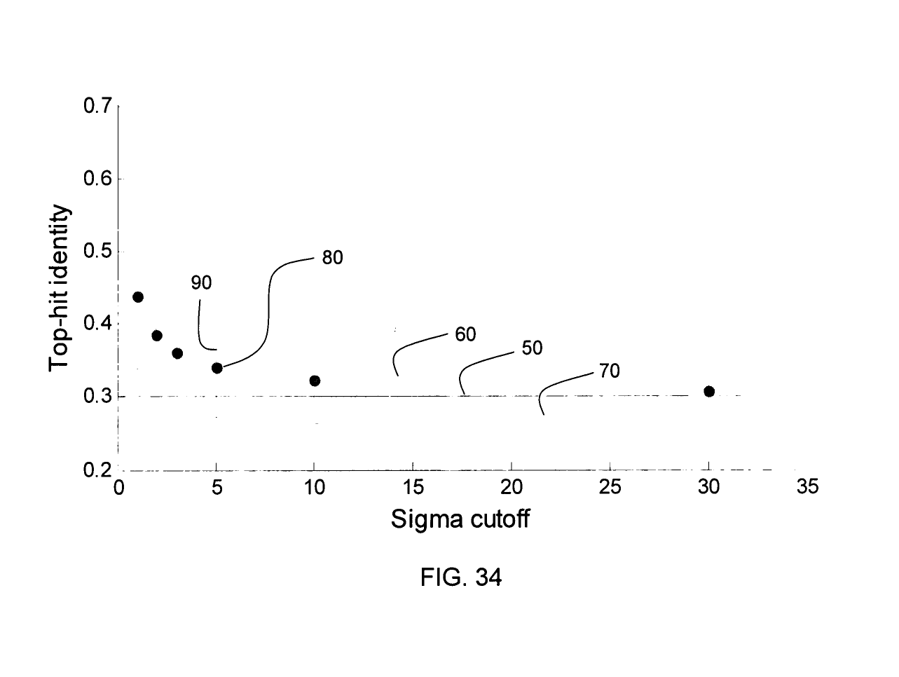

FIG. 34 shows comparable values to those in FIGS. 28 and 31 that were determined using an alignment of natural SH2 sequences.

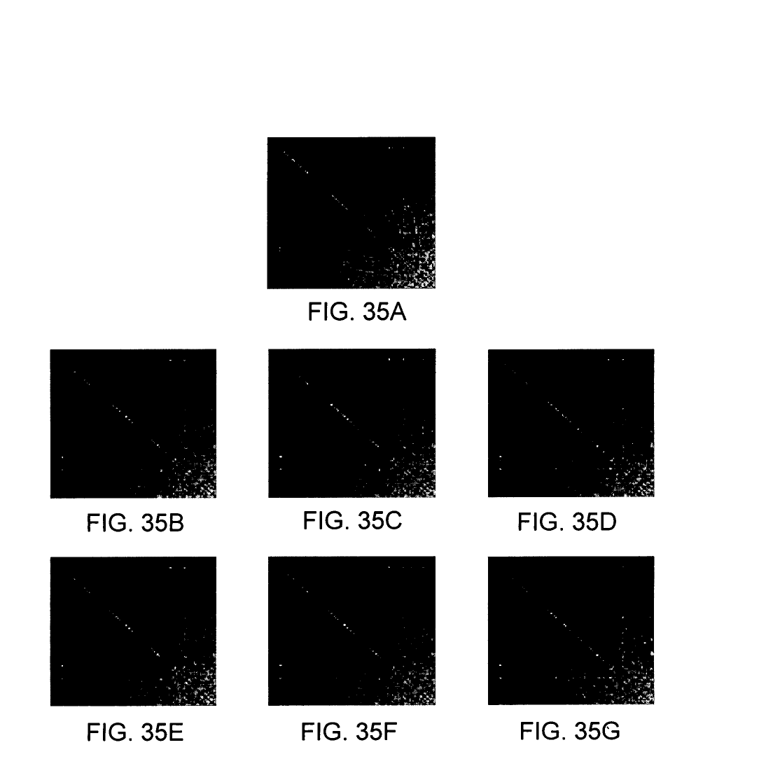

FIG. 35A is a cmr matrix of the natural SH2 alignment. FIGS. 35B-G are each a cmr matrix of the artificial SH2 sequences created using the version of the optimization algorithm described above that includes a mask (which includes SCA-MC-2-mask-AP.c), where the mask was set such that the significance cutoff was chosen as one of the standard deviations above the mean conserved co-evolution score (C^{ev}) of the entire SCM (those standard deviations are 1, 2, 3, 5, 10 and 30).

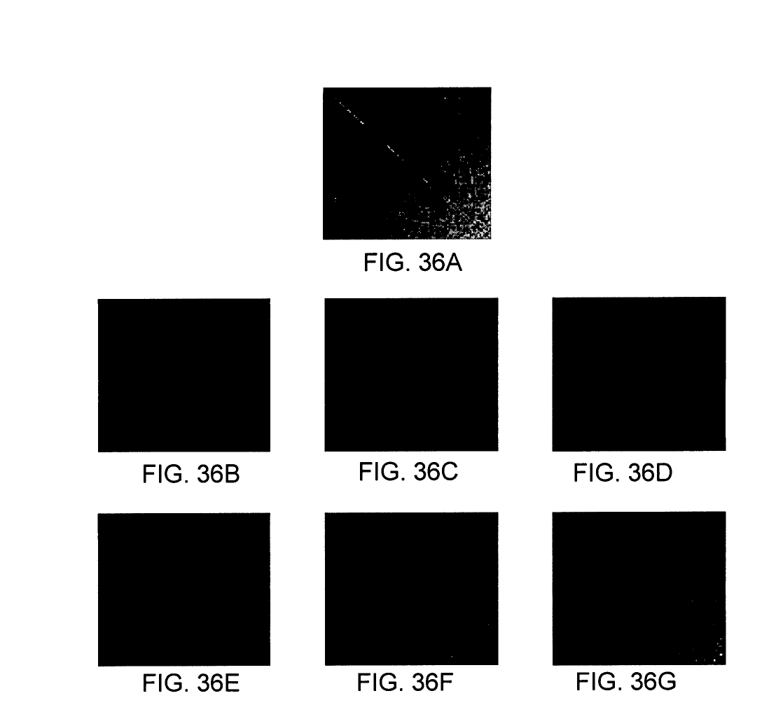

FIGS. 36A-G are included to illustrate the effectiveness of the masking techniques employed. FIG. 36A shows the cmr matrix of the natural SH2 alignment again. FIGS. 36B-G are difference matrices, respectively, between the cmr matrix shown in FIG. 36A and those shown in FIGS. 35B-G.

Gene Construction and Protein Expression

To test the ability of the artificial WW sequences to fold and function like their natural counterparts, genes corresponding to the protein sequences selected from each of the six points along the Monte Carlo trajectory indicated by the red lines in FIG. 19 were constructed, and the expressed proteins were studied. As a positive control, a library of natural WW domains was built because the efficiency of these proteins folding in the experimental laboratory conditions was unknown.

Genes corresponding to the artificial protein sequences were constructed by back-translation (using E. coli codon optimization) built by the polymerase chain reaction (PCR) using overlapping 45-mer oligonucleotide sequences (oligos) that cover each gene. The overlap was adjusted to have a melting temperature (Tm) of ˜60° C. The PCR products were digested at NcoI and XhoI sites encoded on the terminal primers and subcloned into the pHIS8-3 expression vector. Constructs were verified by DNA sequencing.

Proteins were expressed in JM109(DE3) cells grown at 37° C. in Terrific Broth (TB) to an OD600 of ˜1.2, and induced with 0.5 mM IPTG at 18° C. overnight. For fluorescence experiments, 50 ml cultures were lysed in 3 ml binding buffer (BB, 25 mM Tris HCl, pH 8.0, 0.5 M NaCl, 5 mM imidazole) by sonication followed by centrifugation and incubation of the cleared lysate with 75 μl bed volume Ni⁺-NTA (Qiagen) for 30 minutes at 4° C. After washing (3× with 15 ml BB), WW proteins were eluted in elution buffer (EB, 100 mM Tris HCl, pH 8.0, 1 M Na Cl, 400 mM imidazole). Cultures were scaled up for NMR experiments, using 1-2 L of TB and 0.5 ml of affinity resin.

To assess the ability of these artificial proteins to fold into a stable, WW-like structure, thermal denaturation assays were conducted. A hallmark of natively-folded small proteins is cooperative and reversible transition between folded and unfolded states. In the WW domain, this folding equilibrium and the consistency of the two-state approximation have been well-described (Jager et al., 2001; Koepf et al., 1999). Here, the folding reaction was followed by monitoring the fluorescence of a buried tryptophan (Trp7), which becomes quenched due to solvent exposure upon thermal denaturation and therefore reports the fraction of protein folded as a function of temperature.

Experimental Tests of Folding

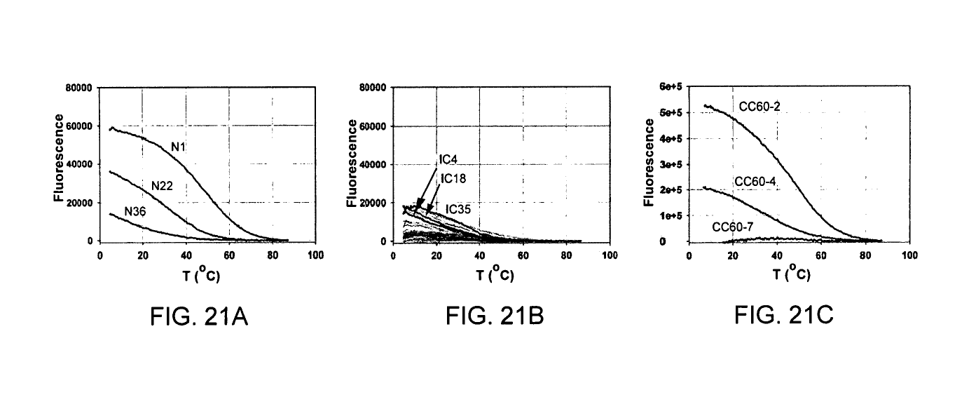

First, 42 natural WW domains were tested in order to determine the frequency of observing folded proteins using this assay. Natural WW domains show a range of thermal denaturation profiles (FIG. 21A). Some such as N1 are clearly well-folded, showing a cooperative denaturation with thermodynamic parameters typical for WW domains (ΔHᵤ^{VH}=22.5 kcal/mol, Tₘ=46.8° C.). Others such as N22 cooperatively denature but are less stable (ΔHᵤ^{VH}=18.5 kcal/mol, Tₘ=25.2° C.). Still others such as N36, despite good solubility, show no convincing evidence of a native state given the experimental conditions of the assay. Twenty-eight of 42 natural WW proteins (67%) were determined to be folded using this assay. As a negative control, a set of sequences was tested from an alignment that strictly preserved the conservation pattern of the, but that contained no coupling information between positions (site Independent Coupling, or IC). In essence, these are the sequences resulting from initial randomization of the natural alignment (described above in the section entitled Designing Artificial Members of a Family of Biological Sequences) but prior to Monte Carlo annealing. Of these 43 IC sequences, none were folded using this assay (FIG. 21B).

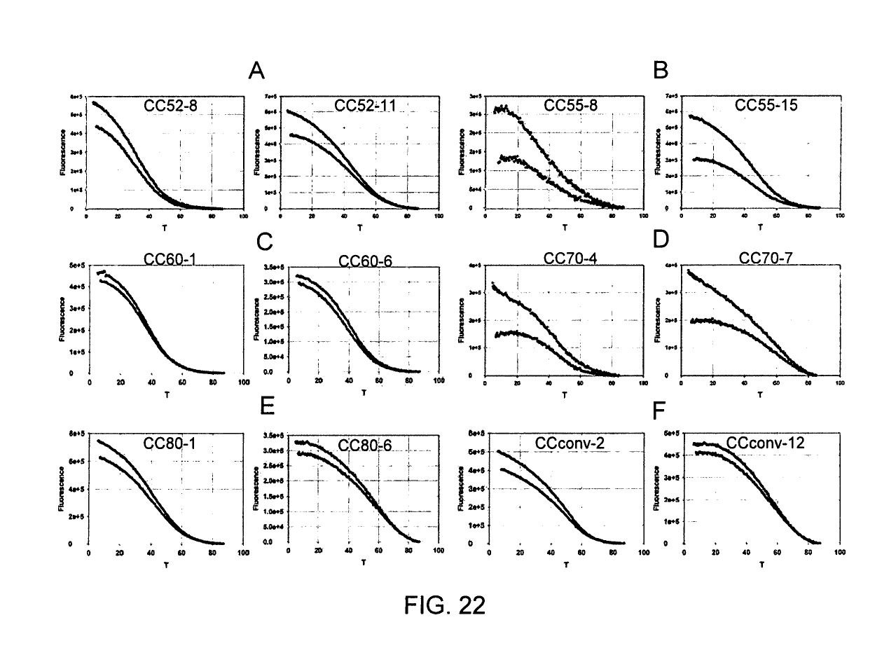

Twenty sequences were selected from each of the six points along the Monte Carlo convergence trajectory (those in FIG. 20 depict cmr matrices of the artificial alignments selected) and tested for folding. FIG. 21C shows examples of the data for these sequences from the 60% identity set. Just as the case of wild-type WW domains, artificial sequences drawn from this stage in the convergence were found to comprise a range of fold stabilities. Importantly, the stability of the folded artificial domains are similar to natural domains (compare FIG. 21A and FIG. 21C). Examples from all of the six sets are shown in FIGS. 22A-F, demonstrating that domains from all groups include sequences that display natural-like folding. Table 1 below summarizes the results for all sets of domains.

TABLE 1
Solubility and folding of natural and artificial WW sequences.

| | average | | | |
|---|---|---|---|---|
| | maximal | | # tested | |
| | identity (%) | energy (eₙ) | (% soluble) | # folded (%) |
| Natural | 100 | 0 | 42 (84%) | 28 (67%) |
| IC | 51 | 23076 | 43 (70%) | 0 (0%) |
| CC52 | 52 | 18114 | 19 (58%) | 4 (21%) |
| CC55 | 55 | 12602 | 20 (50%) | 6 (30%) |
| CC60 | 60 | 8171 | 20 (50%) | 6 (30%) |
| CC70 | 70 | 4528 | 20 (45%) | 6 (30%) |
| CC80 | 80 | 2721 | 20 (75%) | 11 (55%) |
| converged | 84 | 2116 | 20 (75%) | 14 (70%) |

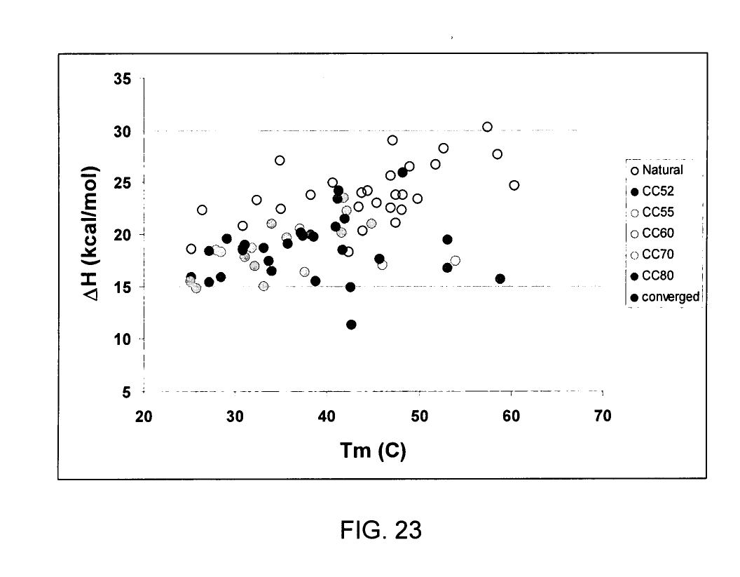

The observed distribution of thermodynamic parameters extracted from these measurements (melting temperature (Tₘ) and the van't Hoff enthalpy of unfolding (ΔH^{VH})) indicate that the designed sequences are quantitatively similar in fold stability to natural domains (see FIG. 23). In fact, even for the CC52 and CC55 sequence sets that show low average maximal identities to natural sequences, it appears that the folded subset have thermodynamic parameters that fall well within the range of natural WW domains. From these results, it was concluded that the information in the SCM is both necessary and sufficient to define the fold of the WW domain. The data show that substantial sequence divergence can be tolerated by the WW fold if the rules of statistical coupling are imposed. This result opens up the possibility of rationally generating enormous libraries of thermally stable proteins that are variants of natural proteins.

Function from Artificial Proteins

Protein sequences evolve through random mutagenesis with selection for optimal fitness. Cooperative folding into a stable tertiary structure is one aspect of fitness, but evolutionary selection ultimately operates on function, not on structure. If indeed an SCM, such as a cma matrix or a cmr matrix, is capturing all of the sequence information for specifying natural-like proteins, then our designed artificial sequences should also function in a manner indistinguishable from that of natural WW domains.

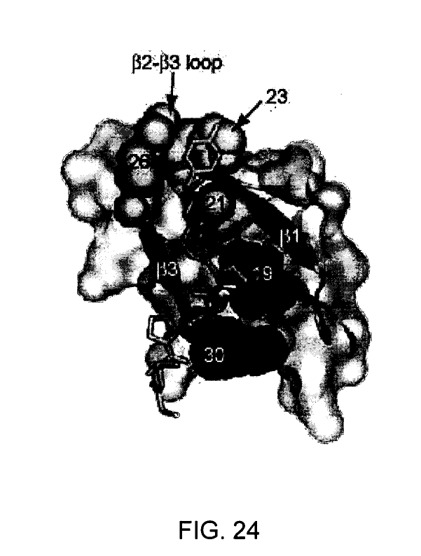

WW domains are small protein interaction modules that adopt a curved three-stranded β-sheet structure with a binding site for proline-containing peptides formed on the concave surface of the sheet (see FIG. 24). The binding surface includes an X-Pro binding site (positions 19 and 30, in blue CPK), which recognizes the canonical proline in target peptides, and a specificity site formed by residues in β2 and the β2-β3 loop (positions 21, 23, and 26, in yellow CPK) (Zarrinpar and Lim, 2000). WW domains are classified into four groups based on target peptide sequence motifs: group I—PPxY (Chen and Sudol, 1995), group II—PPLP Ermckova et al., 1997), group III—PPR (Bedford et al., 2000), and group IV—pS/pT-P (Lu et al., 1999), where x stands for any amino acid.

It was considered whether the amino acid composition at sites plus the information in the SCM comprises sufficient the sequence information to specify the WW domain. The results above provide the first phase of this experimental test by showing that artificial sequences designed using only these parameters adopt a stable WW-like tertiary structure. However, a key further test is the sufficiency of this information for specifying function. If the SCM captures the fitness constraints on the WW family, then the artificial sequences should show class-specific recognition of proline-containing sequences and binding affinities like those of natural WW domains.

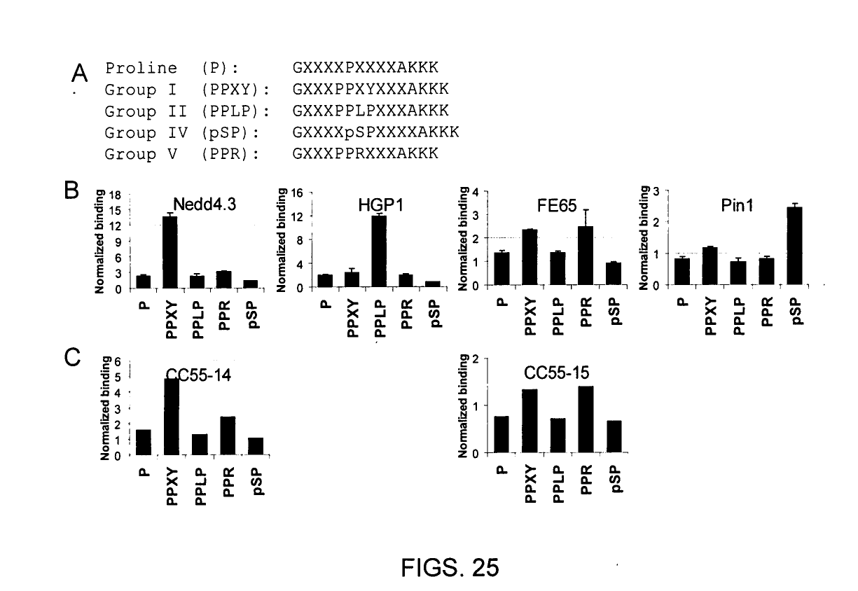

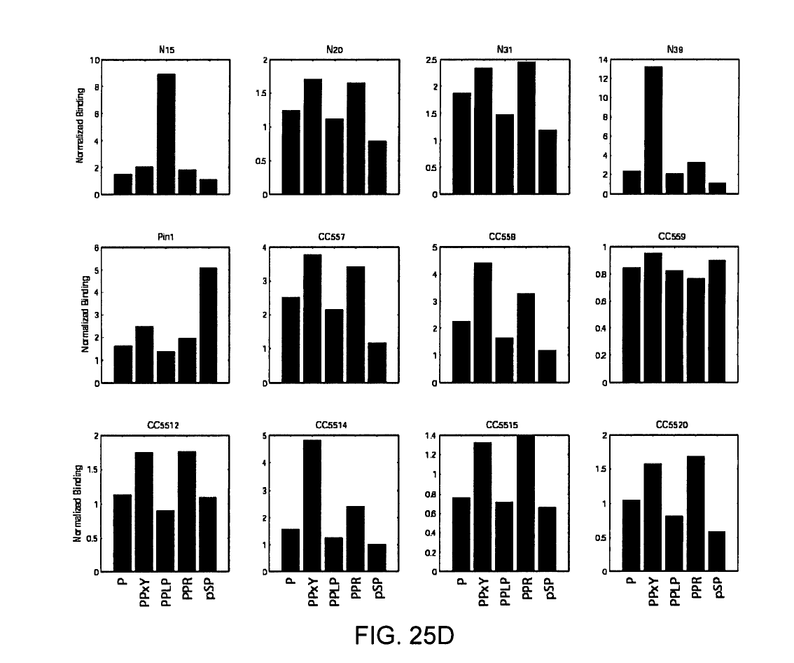

An oriented peptide library binding assay was developed for measuring WW domain specificity, and a set of natural and artificial sequences was studied. Four biotinylated degenerate peptide libraries were constructed, each oriented around one group-specific WW recognition motif, and binding was detected using an ELISA assay (see FIGS. 25A and 25B). For example, the group I oriented peptide library was biotin-Z-GMAxxxPPxYxxxAKKK (SEQ ID NO:163), where Z is 6-aminohexanoic acid and x stands for any amino acid except cysteine (theoretical degeneracy of 8.9×10⁸ sequences). A fifth proline-oriented library was also made as a control for non-specific binding. Natural WW domains that folded in the work described above were subjected to this assay. For a group I domain (Nedd4.3) the peptide library assay confirms specific interaction with the PPxY-containing sequences (Kanelis et al., 2001) (FIG. 25B). Similarly, the assay confirms known group III (FE65) and IV (Pin1) domains. HGP1 is a domain of previously unknown specificity, but clearly shows a preference for the PPLP peptide library, indicating that it has group II specificity.

Analysis of artificial domains from the CC55 set indicates that these domains also bind with specificity (FIGS. 25C-D). CC55-14 (SEQ ID NO:34) binds preferably to the PPXY library, and is classified as a group I domain. Several other domains exhibit the group III binding profile, such as CC55-15 (SEQ ID NO:35). These data demonstrate that WW domains designed using the SCA information in fact function with natural-like ligand binding specificity. The finding that SCA-designed sequences show native-like fold stability and functional specificity suggests that a SCA-based design should enable the creation of large libraries of functional sequences suitable for in-vitro or in-vivo selection.

In sections I and II below, methods are presented for modifying, expressing and purifying artificial biological sequences that can be created using embodiments of the present methods.

Nucleic Acid Vectors and Expression Systems

A variety of nucleic acid vector systems may be used to encode and express artificial polypeptides according to the invention. The term “vector” is used to refer to a carrier nucleic acid molecule into which a nucleic acid sequence can be inserted for introduction into a cell where it can be replicated. The term “expression vector” refers to any type of genetic construct comprising a nucleic acid coding for a RNA capable of being transcribed. In some cases, RNA molecules are then translated into a protein, polypeptide, or peptide such as artificial polypeptide sequences described herein.

Expression vectors can contain a variety of “control sequences,” which refer to nucleic acid sequences necessary for the transcription and possibly translation of an operably linked coding sequence in a particular host cell. Control sequences include but are not limited to transcription promoters, and enhancers, RNA splice sites, polyadenylation signal sequences, and ribosome binding sites. Some promoters and enhancers are exemplified in the Eukaryotic Promoter Data Base EPDB, (http://www.epd.isb-sib.ch/) and could be used to drive expression of desired sequences.

Vectors may also comprise selectable markers, such as drug selection marker that enable selection of cells expressing a desired nucleic acid/polypeptide sequence. For example, genes that confer resistance to ampicillin, kanamycin, chloroamphenicol, neomycin, puromycin, hygromycin, blastacidin, DHFR, GPT, zeocin and histidinol are useful selectable markers.

Some specific vectors that are contemplated herein are viral vectors that enable the highly efficient transformation of eukaryotic cells via the natural infection process of some viruses. Viral vectors are well know to those of skill in the art and some of the best characterized systems are the adenoviral, adeno-associated viral, retroviral, and vaccinia viral vector systems.

In addition to delivery of nucleic acid to cells via viral vectoring, a variety of other methods for delivery for nucleic acids into cells are well known in those in the art. Some examples include but are not limited to, electroporation of cells, chemical transfection (e.g., with calcium phosphate or DEAE-dextran), liposomal delivery or microprojectile bombardment.

Polypeptide Expression and Purification

It will be understood to by one of skill in the art that artificial polypeptides according to the invention may be chemically synthesized or expressed in cells and purified. Generally, “purified” will refer to an artificial protein that has been subjected to fractionation or isolation to remove various other protein or peptide components. To purify recombinantly expressed artificial polypeptides, cell lysates from expressing cells will be subjected to fractionation to remove various other components from the composition. Various techniques suitable for use in protein purification will be well known to those of skill in the art. In some cases artificial polypeptides may be fused with additional amino acid sequence such sequences may, for example, facilitate polypeptide purification. Some possible fusion proteins that could be generated include histadine tags (as specifically exemplified herein), Glutathione S-transferase (GST), Maltose binding protein (MBP), Flag and myc tagged artificial polypeptides. These additional sequences may be used to aid in purification of the recombinant protein, and in some cases may then be removed by protease cleavage.

The claims are not to be interpreted as including means-plus- or step-plus-function limitations, unless such a limitation is explicitly recited in a given claim using the phrase(s) “means for” or “step for,” respectively.

### REFERENCES

The following references, to the extent that they provide procedural or other details supplementary to those set forth in this disclosure, are specifically incorporated by reference.
Altschul et al. Nucleic Acids Res., 25:3389-402, 1997.
Anfinsen, Science, 181:223-30, 1973.
Bedford et al., J. Biol. Chem., 275:10359-10369, 2000.
Brown, Nucleic Acids Res., 27(1):314, 1999.
Carter et al., Cell, 38:835-840, 1984.
Chen and Stites, Biochemistry, 40:14012-9, 2001.
Chen and Sudol, Proc. Natl. Acad. Sci. USA, 92:7819-7823, 1995.
Chen et al., J. Biol. Chem., 272:17070-17077, 1997.
Daggett and Fersht, Nat. Rev. Mol. Cell. Biol., 4:497-502, 2003.
Dinner et al., Trends Biochem. Sci., 25:331-9, 2000.
Dobson, Nature, 426:884-90, 2003.
Ermekova et al., J. Biol. Chem., 272:32869-32877, 1997.
Espanel and Sudol, J. Biol. Chem., 274:17284-17289, 1999.
Fersht and Daggett, Cell, 108:573-82, 2002.
Garrard et al., Embo. J, 22:1125-1133, 2003.
Gerstein and Chothia, Proc. Natl. Acad. Sci. U.S.A., 93:10167-72, 1996.
Hardy et al., Proc. Natl. Acad. Sci. USA, 101:12461-12466, 2004.
Hidalgo and MacKinnon, Science, 268:307-10, 1995.
Hidalgo and MacKinnon, Science, 268:307-310, 1995.
Horovitz and Fersht, J. Mol. Biol., 224:733-40, 1992.
Huang et al., Nat. Struct. Biol., 7:634-638, 2000.
Hubbard et al., Nucleic Acids Res., 27(1):254-256, 1999.
Jager et al., J. Mol. Biol., 311:373-393, 2001.
Kanelis et al., Nat. Struct. Biol., 8:407-412, 2001.
Kasanov et al., Chem. Biol., 8:231-241, 2001.
Klingler and Brutlag, Protein Sci., 3:1847-57, 1994.
Koepf et al., Protein Sci., 8:841-853, 1999.
LiCata and Ackers, Biochemistry, 34:3133-9, 1995.
Lockless and Ranganathan, Science, 286:295-299, 1999.
Lowe and Eddy, Nucleic Acids Res., 25(5):955-964, 1997.
Lu et al., Science, 283:1325-1328, 1999.
Luque et al., Annu. Rev. Biophys. Biomol. Struct., 31:235-56, 2002.
Marqusee and Sauer, Protein Sci., 3:2217-25, 1994.
Pei et al., Bioinformatics. 19(3):427-8, 2003.
Peterson et al., Mol. Cell, 13:665-676, 2004.
Rath et al., Chem. Biol., 7:677-682, 2000.
Richards and Lim, Q. Rev. Biophys., 26:423-98, 1993.
Skelton et al., J. Biol. Chem., 278:7645-7654, 2003.
Suel et al., Nat. Struct. Biol., 10:59-69, 2003.
Thompson et al., Nucl. Acids Res., 22:4673-80, 1994.
Toepert et al., Angew Chem. Int. Ed. Engl., 40:897-900, 2001.
Zarrinpar and Lim, Nat. Struct. Biol., 7:611-613, 2000.
Zwieb, Nucl. Acids Res., 1997, 25(1):102-103, 1997.

### Computer Program Listings

The following computer program listings are organized by file name, which is centered above the listing to which it applies:

### random_elim_dg.m

function [data_out]=random_elim_dg(A)

% initial matters

[nseqs,y]=size(A);

nreps=100;

f_elim=0.03;

site_parent=zeros(y,20);

site_new=zeros(y,20);

% calculation of conservation at sites, sorting of the conservation

% values low to high, and choosing the five sites with lowest conservation

% values but that have at least 85% representation in the alignment.

We claim:

1. A method comprising:
(a) testing the size and diversity of an alignment of a family of M biological sequences, each biological sequence having N positions, each position being occupied by one biological position element of a group of biological position elements;
(b) calculating a statistical conservation value for each biological position element in a pair of biological position elements at different positions in the alignment; and
(c) measuring conserved co-variation between the biological position elements in the pair using the statistical conservation values.

2. The method of claim 1, where the calculating of (b) comprises calculating a statistical conservation value for each biological position element at each position in the alignment.

3. The method of claim 2, where the measuring of (c) comprises measuring conserved co-variation between every pair of biological position elements at every pair of positions in the alignment using the statistical conservation values.

4. The method of claim 2, where the calculating of (b) comprises calculating a statistical conservation value ΔE_{i,x}^{stat} for each biological position element x in the pair of biological statistical elements at different positions i in the alignment using the following equation:
$$Δ {E}_{i , x}^{stat} = {γ}^{*} ln ( \frac{{P}_{i}^{x}}{{P}_{align}^{x}} ) ,$$
where Pᵢ^{x} is the probability of biological position element x at position i;
P_{align}^{x} is the probability of biological position element x in the alignment; and
γ* is an arbitrary statistical energy unit.

5. The method of claim 4, where Pᵢ^{x} is calculated using a binomial density function as set forth in the following equation:
$${P}_{i}^{x} = \frac{z !}{( {zf}_{i}^{x} ) ( z - {zf}_{i}^{x} )} {{p}_{x}^{{zf}_{i}^{x}} ( 1 - {p}_{x} )}^{z - {zf}_{i}^{x}} ,$$
where
$${f}_{i}^{x} = \frac{{m}_{i}^{x}}{M} , {m}_{i}^{x}$$
is the number of biological position elements x at position i in the alignment, z is a normalization factor, zfᵢ^{x} is the number of biological sequences having biological position element x in a normalized version of the alignment having z biological sequences, and pₓ is a baseline mean frequency of biological position element x.

6. The method of claim 5, where z is 100.

7. The method of claim 2, where the calculating of (b) comprises calculating a statistical conservation value ΔE_{i,x}^{stat} for each biological position element x at each position i in the alignment using the following equation:
$$Δ {E}_{i , x}^{stat} = {γ}^{*} ln ( \frac{{P}_{i}^{x}}{{P}_{align}^{x}} ) ,$$
where Pᵢ^{x} is the probability of biological position element x at position i;
P_{align}^{x} is the probability of biological position element x in the alignment; and
γ* is an arbitrary statistical energy unit.

8. The method of claim 7, where Pᵢ^{x} is calculated using a binomial density function as set forth in the following equation:
$${P}_{i}^{x} = \frac{z !}{( {zf}_{i}^{x} ) ( z - {zf}_{i}^{x} )} {{p}_{x}^{{zf}_{i}^{x}} ( 1 - {p}_{x} )}^{z - {zf}_{i}^{x}} ,$$
where
$${f}_{i}^{x} = \frac{{m}_{i}^{x}}{M} , {m}_{i}^{x}$$
is the number of biological position elements x at position i in the alignment, z is a normalization factor, zfᵢ^{x} is the number of biological sequences having biological position element x in a normalized version of the alignment having z biological sequences, and pₓ is a baseline mean frequency of biological position element x.

9. The method of claim 8, where z is 100.

10. The method of claim 7, further comprising:
(d) perturbing the alignment by eliminating each biological sequence from the alignment such that M subalignments each having M−1 sequences are created.

11. The method of claim 10, where the measuring of (c) comprises calculating a change in statistical conservation of each biological position element at each position after each elimination of a biological sequence from the alignment.

12. The method of claim 10, where the measuring of (c) comprises:
(i) calculating a statistical conservation value ΔE_{i,x}^{stat} for each biological position element x at each position i in each of the M subalignments after each elimination of a biological sequence from the alignment; and
(ii) calculating a statistical conservation difference value ΔΔE_{i,x,δm}^{stat} after (i) using
$$ΔΔ {E}_{i , x , δ m}^{stat} = ( Δ {E}_{i , x}^{stat} - Δ {E}_{i , x \vert δ m}^{stat} ) * \frac{M}{z} ,$$
where δm signifies a given elimination of one biological sequence from the alignment;
ΔE_{i,x,δm}^{stat} is calculated for each biological position element x at each position i in each subalignment in the same manner as the statistical conservation values ΔE_{i,x}^{stat} in claim 7; and
M/z is a normalization factor.

13. The method of claim 12, where z comprises 100.

14. The method of claim 12, where the measuring of (c) further comprises:
(iii) arranging the statistical conservation difference values ΔΔE_{i,x,δm}^{stat} into a perturbation vector \vec{ΔΔE}_{i,x}^{stat} for each biological position element x at each position i in the alignment, where
$${\vec{ΔΔ E}}_{i , x}^{stat} = \{ Δ Δ {E}_{i , x , δ m 1}^{stat} , Δ Δ {E}_{i , x , δ m 2}^{stat} , … , Δ Δ {E}_{i , x , δ m M}^{stat} \} .$$

15. The method of claim 12, where the perturbing of (d) creates M-dimensional space, and the measuring of (c) further comprises:
(iv) calculating a statistical coupling value C_{i,x,j,y}^{ev} for each pair of biological position elements x and y at each pair of positions i and j in the alignment, using
$${C}_{i , x , j , y}^{ev} = {\vec{ΔΔ E}}_{i , x}^{stat} \cdot {\vec{ΔΔ E}}_{j , y}^{stat} ,$$
where
$${\vec{ΔΔ E}}_{i , x}^{stat} \cdot {\vec{ΔΔ E}}_{j , y}^{stat} = \vert {\vec{ΔΔ E}}_{i , x}^{stat} \vert \vert {\vec{ΔΔ E}}_{j , y}^{stat} \vert cos θ ,$$
and θ is the angle between perturbation vectors \vec{ΔΔE}_{i,x}^{stat} and \vec{ΔΔE}_{j,y}^{stat} in the M-dimensional space.

16. The method of claim 12, further comprising:
(e) arranging the statistical coupling values into a statistical coupling matrix (SCM) having dimensions N×N×r×r, where r represents the number of biological position elements in the group.

17. The method of claim 16, where the biological sequences comprise proteins, the biological position elements comprise amino acids, and r comprises 20.

18. The method of claim 16, where the biological sequences comprise nucleic acid sequences, the biological position elements comprise nucleic acids, and r comprises 4.

19. The method of claim 16, further comprising:
(f) designing artificial biological sequences using the SCM.

20. The method of claim 16, further comprising:
(f) designing artificial biological sequences using a subset of the SCM.

21. The method of claim 19, where the M biological sequences in the alignment are functionally organized in M rows and N columns, and the designing of (f) comprises:
(i) randomizing the alignment to yield a randomized alignment that retains M biological sequences in M rows and N columns;
(ii) iteratively altering the randomized alignment to yield altered alignments;
(iii) creating a statistical coupling matrix for each altered alignment; and
(iv) determining whether to accept an alteration.

22. The method of claim 21, where the determining of (f)(iv) comprises using an optimization algorithm in determining whether to accept an alteration.

23. The method of claim 22, where the optimization algorithm comprises a simulated annealing algorithm.

24. The method of claim 21, where the alignment has a conservation pattern, and the randomizing of (f)(i) substantially preserves the conservation pattern.

25. The method of claim 19, where the M biological sequences in the alignment are functionally organized in M rows and N columns, the SCM comprises an SCM_{target}, and the designing of (f) comprises:
(i) randomizing the alignment to yield a randomized alignment that retains M biological sequences in M rows and N columns, the randomized alignment comprising an iteration 0 alignment (alignment₀);
(ii) obtaining a statistical coupling matrix SCM₀ for alignment₀;
(iii) swapping two biological position elements within one column of the randomized alignment to yield an alignmentₙ, where each swapping comprises an iteration, and n comprises the number of iterations;
(iv) obtaining a statistical coupling matrix SCMₙ for the alignmentₙ;
(v) obtaining a scalar system energy value eₙ, where eₙ=|SCMₙ−SCM_{target}|;
(vi) obtaining a scalar system energy value difference Δe, where Δe=eₙ−eₙ₋₁; and
(vii) determining whether to accept a swapping from (f)(ii) for a given iteration, where the determining comprises:
(1) if Δe≦0, accepting the swapping of (f)(ii) for the given iteration; and
(2) if Δe>0, accepting the swapping of (f)(ii) for the given iteration with a non-zero probability; and
(viii) repeating (f)(ii)-(f)(vii) until a termination criteria is satisfied.

26. The method of claim 25, where the non-zero probability comprises
$$exp ( \frac{- Δ e}{β} )$$
and β decreases.

27. The method of claim 26, where β decreases according to the number of acceptances (f)(vi)(1) or (f)(vi)(2).

28. The method of claim 25, where the termination criteria is based on the frequency of acceptances (f)(vii)(1) or (f)(vii)(2) relative to the number of swappings attempted.

29. The method of claim 25, where the alignment has a conservation pattern, and the randomizing of (f)(i) substantially preserves the conservation pattern.

30. The method of claim 25, where the biological sequences comprise proteins, the biological position elements comprise amino acids, and r comprises 20.

31. The method of claim 25, where the biological sequences comprise nucleic acid sequences, the biological position elements comprise nucleic acids, and r comprises 4.

32. The method of claim 25, where the scalar system energy values form a convergence trajectory relative to the number of iterations, and the designing of (f) further comprises:
(ix) selecting artificial biological sequences at different points along the convergence trajectory.

33. The method of claim 15, where the biological sequences in the alignment are part of a family of biological sequences, and the method further comprises:
(e) reducing the C_{i,x,j,y}^{ev} values to C_{i,j}^{ev} values; and
(f) using a clustering algorithm to group positions with similar C_{i,j}^{ev} values.

34. The method of claim 33, where the clustering algorithm comprises a hierarchal clustering algorithm.

35. The method of claim 33, further comprising:
(g) mapping the grouped positions on a representation of a 3D structure of a biological sequence in the alignment.

36. A method comprising:
(a) calculating a statistical conservation value for each biological position element in a pair of biological position elements at different positions in an alignment of a family of M biological sequences, each biological sequence having N positions, and each position being occupied by one biological position element of a group of biological position elements; and
(b) measuring conserved co-variation between the biological position elements in the pair using the statistical conservation values.

37. The method of claim 36, where the calculating of (a) comprises calculating a statistical conservation value for each biological position element at each position in the alignment.

38. The method of claim 37, where the measuring of (b) comprises measuring conserved co-variation between every pair of biological position elements at every pair of positions in the alignment using the statistical conservation values.

39. The method of claim 37, where the calculating of (a) comprises calculating a statistical conservation value ΔE_{i,x}^{stat} for each biological position element x in the pair of biological statistical elements at different positions i in the alignment using the following equation:
$$Δ {E}_{i , x}^{stat} = {γ}^{*} ln ( \frac{{P}_{i}^{x}}{{P}_{align}^{x}} ) ,$$
where Pᵢ^{x} the probability of biological position element x at position i;
P_{align}^{x} is the probability of biological position element x in the alignment; and
γ* is an arbitrary statistical energy unit.

40. The method of claim 39, where Pᵢ^{x} is calculated using a binomial density function as set forth in the following equation:
$${P}_{i}^{x} = \frac{z !}{( {zf}_{i}^{x} ) ( z - {zf}_{i}^{x} )} {{p}_{x}^{{zf}_{i}^{x}} ( 1 - {p}_{x} )}^{z - {zf}_{i}^{x}} ,$$
where
$${f}_{i}^{x} = \frac{{m}_{i}^{x}}{M} ,$$
mᵢ^{x} is the number of biological position elements x at position i in the alignment, z is a normalization factor, zfᵢ^{x} is the number of biological sequences having biological position element x in a normalized version of the alignment having z biological sequences, and pₓ is a baseline mean frequency of biological position element x.

41. The method of claim 40, where z is 100.

42. The method of claim 37, where the calculating of (a) comprises calculating a statistical conservation value ΔE_{i,x}^{stat} for each biological position element x at each position i in the alignment using the following equation:
$$Δ {E}_{i , x}^{stat} = {γ}^{*} ln ( \frac{{P}_{i}^{x}}{{P}_{align}^{x}} ) ,$$
where Pᵢ^{x} is the probability of biological position element x at position i;
P_{align}^{x} is the probability of biological position element x in the alignment; and
γ* is an arbitrary statistical energy unit.

43. The method of claim 42, where Pᵢ^{x} is calculated using a binomial density function as set forth in the following equation:
$${P}_{i}^{x} = \frac{z !}{( {zf}_{i}^{x} ) ( z - {zf}_{i}^{x} )} {{p}_{x}^{{zf}_{i}^{x}} ( 1 - {p}_{x} )}^{z - {zf}_{i}^{x}} ,$$
where
$${f}_{i}^{x} = \frac{{m}_{i}^{x}}{M} , {m}_{i}^{x}$$
is the number of biological position elements x at position i in the alignment, z is a normalization factor, zfᵢ^{x} is the number of biological sequences having biological position element x in a normalized version of the alignment having z biological sequences, and pₓ is a baseline mean frequency of biological position element x.

44. The method of claim 43, where z is 100.

45. The method of claim 42, further comprising:
(c) perturbing the alignment by eliminating each biological sequence from the alignment such that M subalignments each having M−1 sequences are created.

46. The method of claim 45, where the measuring of (b) comprises calculating a change in statistical conservation of each biological position element at each position after each elimination of a biological sequence from the alignment.

47. The method of claim 45, where the measuring of (b) comprises:
(i) calculating a statistical conservation value ΔE_{i,x}^{stat} for each biological position element x at each position i in each of the M subalignments after each elimination of a biological sequence from the alignment; and
(ii) calculating a statistical conservation difference value ΔΔE_{i,x,δm}^{stat} after (i) using
$$ΔΔ {E}_{ i , x , δ m}^{ stat} = ( Δ {E}_{ i , x}^{ stat} - Δ {E}_{ i , x \vert δ m}^{ stat} ) * \frac{M}{ z} ,$$
where δm signifies a given elimination of one biological sequence from the alignment;
ΔE_{i,x,δm}^{stat} is calculated for each biological position element x at each position i in each subalignment in the same manner as the statistical conservation values ΔE_{i,x}^{stat} in claim 42; and
M/z is a normalization factor.

48. The method of claim 47, where z comprises 100.

49. The method of claim 47, where the measuring of (b) further comprises:
(iii) arranging the statistical conservation difference values ΔΔE_{i,x,δm}^{stat} into a perturbation vector \vec{ΔΔE}_{i,x}^{stat} for each biological position element x at each position i in the alignment, where
$${\vec{ΔΔ E}}_{i , x}^{stat} = \{ Δ Δ {E}_{i , x , δ m 1}^{stat} , Δ Δ {E}_{i , x , δ m 2}^{stat} , … , Δ Δ {E}_{i , x , δ m M}^{stat} \} .$$

50. The method of claim 47, where the perturbing of (c) creates M-dimensional space, and the measuring of (b) further comprises:
(iv) calculating a statistical coupling value C_{i,x,j,y}^{ev} for each pair of biological position elements x and y at each pair of positions i and j in the alignment, using
$${C}_{i , x , j , y}^{ev} = {\vec{ΔΔ E}}_{i , x}^{stat} \cdot {\vec{ΔΔ E}}_{j , y}^{stat} ,$$
where
$${\vec{ΔΔ E}}_{i , x}^{stat} \cdot {\vec{ΔΔ E}}_{j , y}^{stat} = \vert {\vec{ΔΔ E}}_{i , x}^{stat} \vert \vert {\vec{ΔΔ E}}_{j , y}^{stat} \vert cos θ ,$$
and θ is the angle between perturbation vectors \vec{ΔΔE}_{i,x}^{stat} and \vec{ΔΔE}_{j,y}^{stat} in the M-dimensional space.

51. The method of claim 47, further comprising:
(d) arranging the statistical coupling values into a statistical coupling matrix (SCM) having dimensions N×N×r×r, where r represents the number of biological position elements in the group.

52. The method of claim 51, where the biological sequences comprise proteins, the biological position elements comprise amino acids, and r comprises 20.

53. The method of claim 51, where the biological sequences comprise nucleic acid sequences, the biological position elements comprise nucleic acids, and r comprises 4.

54. The method of claim 51, further comprising:
(e) designing artificial biological sequences using the SCM.

55. The method of claim 51, further comprising:
(e) designing artificial biological sequences using a subset of the SCM.

56. The method of claim 54, where the M biological sequences in the alignment are functionally organized in M rows and N columns, and the designing of (e) comprises:
(i) randomizing the alignment to yield a randomized alignment that retains M biological sequences in M rows and N columns;
(ii) iteratively altering the randomized alignment to yield altered alignments;
(iii) creating a statistical coupling matrix for each altered alignment; and
(iv) determining whether to accept an alteration.

57. The method of claim 56, where the determining of (e)(iv) comprises using an optimization algorithm in determining whether to accept an alteration.

58. The method of claim 57, where the optimization algorithm comprises a simulated annealing algorithm.

59. The method of claim 56, where the alignment has a conservation pattern, and the randomizing of (e)(i) substantially preserves the conservation pattern.

60. The method of claim 54, where the M biological sequences in the alignment are functionally organized in M rows and N columns, the SCM comprises an SCM_{target}, and the designing of (e) comprises:
(i) randomizing the alignment to yield a randomized alignment that retains M biological sequences in M rows and N columns, the randomized alignment comprising an iteration 0 alignment (alignment₀);
(ii) obtaining a statistical coupling matrix SCM₀ for alignment₀;
(iii) swapping two biological position elements within one column of the randomized alignment to yield an alignmentₙ, where each swapping comprises an iteration, and n comprises the number of iterations;
(iv) obtaining a statistical coupling matrix SCMₙ for the alignmentₙ;
(v) obtaining a scalar system energy value eₙ, where eₙ=|SCMₙ−SCM_{target}|;
(vi) obtaining a scalar system energy value difference Δe, where Δe=eₙ−eₙ₋₁; and
(vii) determining whether to accept a swapping from (e)(ii) for a given iteration, where the determining comprises:
(1) if Δe≦0, accepting the swapping of (e)(ii) for the given iteration; and
(2) if Δe>0, accepting the swapping of (e)(ii) for the given iteration with a non-zero probability; and
(viii) repeating (e)(ii)-(e)(vii) until a termination criteria is satisfied.

61. The method of claim 60, where the non-zero probability comprises
$$exp ( \frac{- Δ e}{β} )$$
and β decreases.

62. The method of claim 61, where β decreases according to the number of acceptances (e)(vi)(1) or (e)(vi)(2).

63. The method of claim 60, where the termination criteria is based on the frequency of acceptances (e)(vii)(1) or (e)(vii)(2) relative to the number of swappings attempted.

64. The method of claim 60, where the alignment has a conservation pattern, and the randomizing of (e)(i) substantially preserves the conservation pattern.

65. The method of claim 60, where the biological sequences comprise proteins, the biological position elements comprise amino acids, and r comprises 20.

66. The method of claim 60, where the biological sequences comprise nucleic acid sequences, the biological position elements comprise nucleic acids, and r comprises 4.

67. The method of claim 60, where the scalar system energy values form a convergence trajectory relative to the number of iterations, and the designing of (e) further comprises:
(ix) selecting artificial biological sequences at different points along the convergence trajectory.

68. The method of claim 50, where the biological sequences in the alignment are part of a family of biological sequences, and the method further comprises:
(d) reducing the C_{i,x,j,y}^{ev} values to C_{i,j}^{ev} values; and
(e) using a clustering algorithm to group positions with similar C_{i,j}^{ev} values.

69. The method of claim 68, where the clustering algorithm comprises a hierarchal clustering algorithm.

70. The method of claim 68, further comprising:
(f) mapping the grouped positions on a representation of a 3D structure of a biological sequence in the alignment.

71. A method comprising:
(a) testing the size and diversity of an alignment of a family of M biological sequences, each biological sequence having N positions, each position being occupied by one biological position element of a group of biological position elements;
(b) calculating a statistical conservation value for each biological position element in a pair of biological position elements at different positions in the alignment;
(c) making a perturbation to the alignment that is not based on the conservation of a particular biological position element at a particular position, the perturbation yielding a subalignment having fewer than M biological sequences; and
(d) calculating a statistical conservation value for each biological position element in a pair of biological position elements at the different positions in the subalignment.

72. The method of claim 71, further comprising:
(e) calculating a dataset of values using the calculated statistical conservation values from (c) and (d), the values in the dataset being organizable into a square statistical coupling matrix.

73. The method of claim 72, further comprising:
(f) designing one or more artificial biological sequences using the square statistical coupling matrix.

74. The method of claim 72, further comprising:
(f) designing one or more artificial biological sequences using a subset of the square statistical coupling matrix.

75. The method of claim 72, where the square statistical coupling matrix is also symmetric.

76. A method comprising:
(a) calculating a statistical conservation value for each biological position element in a pair of biological position elements at different positions in an alignment of a family of M biological sequences, each biological sequence having N positions, and each position being occupied by one biological position element of a group of biological position elements;
(b) making a perturbation to the alignment that is not based on the conservation of a particular biological position element at a particular position, the perturbation yielding a subalignment having fewer than M biological sequences; and
(c) calculating a statistical conservation value for each biological position element in a pair of biological position elements at the different positions in the subalignment.

77. The method of claim 76, further comprising:
(d) calculating a dataset of values using the calculated statistical conservation values from (b) and (c), the values in the dataset being organizable into a square statistical coupling matrix.

78. The method of claim 77, further comprising:
(e) designing one or more artificial biological sequences using the square statistical coupling matrix.

79. The method of claim 77, further comprising:
(e) designing one or more artificial biological sequences using a subset of the square statistical coupling matrix.

80. The method of claim 77, where the square statistical coupling matrix is also symmetric.

81. A computer readable medium comprising machine readable instructions for executing the steps of any of the methods of claims 1-80.

82. A computer system programmed to execute the steps of any of the methods of claims 1-80.
